(19) United States
(12) Patent Application Publication
(10) Pub. No.: US 2010/0056447 A1
(43) Pub. Date: Mar. 4, 2010

(54) IMMUNIZATION AGAINST CHLAMYDIA PNEUMONIAE

(75) Inventors: Giulio RATTI, Siena (IT); Guido GRANDI, Siena (IT)

Correspondence Address: NOVARTIS VACCINES AND DIAGNOSTICS INC., INTELLECTUAL PROPERTY- X100B, P.O. BOX 8097, Emeryville, CA, 94662-8097, US

(73) Assignee: NOVARTIS AG, Basel (CH)

(21) Appl. No.: 12/543,535

(22) Filed: Aug. 19, 2009

Related U.S. Application Data
(62) Division of application No. 11/414,403, filed on May 1, 2006.
Application No. 11/414,403 is a continuation of application No. 10/312,273, filed on May 5, 2003, now abandoned.

(30) Foreign Application Priority Data
Jul. 3, 2000 (GB) 0016363.4
Jul. 11, 2000 (GB) 0017047.2
Jul. 21, 2000 (GB) 0017938.8
Aug. 7, 2000 (GB) 0019368.0
Aug. 18, 2000 (GB) 0020440.4
Sep. 14, 2000 (GB) 0022583.9
Nov. 10, 2000 (GB) 0027549.5
Dec. 22, 2000 (GB) 0031706.5

(51) Int. Cl. A61K 38/16; C07K 14/00; C07H 21/00; C12N 5/00; A61P 37/04

(52) U.S. Cl. 514/12; 530/350; 536/23.7; 435/375

(57) ABSTRACT

The published genomic sequence of Chlamydia pneumoniae reveals over 1000 putative encoded proteins but does not itself indicate which of these might be useful antigens for immunization and vaccination or for diagnosis. This difficulty is addressed by the invention, which provides a number of C. pneumoniae protein sequences suitable for vaccine production and development and/or for diagnostic purposes.

This application is a division of Ser. No. 11/414,403 filed on May 1, 2006, which is a continuation of Ser. No. 10/312,273 filed on May 5, 2003, now abandoned, which is a national phase application of PCT/IB01/01445 filed on Jul. 3, 2001, which claims priority to GB applications 0016363.4 filed Jul. 3, 2000; 0017047.2 filed Jul. 11, 2000; 0017983.8 filed Jul. 21, 2000; 0019368.0 filed Aug. 7, 2000; 0020440.4 filed Aug. 18, 2000; 0022583.9 filed Sep. 14, 2000; 0027549.5 filed Nov. 10, 2000; and 0031706.5 filed Dec. 22, 2000. Each of these applications is incorporated herein by reference its entirety.

This application incorporates by reference a 949 kb text file created on Aug. 18, 2009 and named “sequencelisting.txt,” which is the sequence listing for this application.

All documents cited herein are incorporated by reference in their entirety.

### TECHNICAL FIELD

This invention is in the field of immunization against chlamydial infection, in particular against infection by Chlamydia pneumoniae.

### BACKGROUND ART

Chlamydiae are obligate intracellular parasites of eukaryotic cells which are responsible for endemic sexually transmitted infections and various other disease syndromes. They occupy an exclusive eubacterial phylogenic branch, having no close relationship to any other known organisms—they are classified in their own order (Chlamydiales) which contains a single family (Chlamydiaceae) which in turn contains a single genus (Chlamydia). A particular characteristic of the Chlamydiae is their unique life cycle, in which the bacterium alternates between two morphologically distinct forms: an extracellular infective form (elementary bodies, EB) and an intracellular non-infective form (reticulate bodies, RB). The life cycle is completed with the re-organization of RB into EB, which subsequently leave the disrupted host cell ready to infect further cells.

Four chlamydial species are currently known—C. trachomatis, C. pneumoniae, C. pecorum and C. psittaci [e.g. Raulston (1995) Mol Microbiol 15:607-616; Everett (2000) Vet Microbiol 75:109-126]. C. pneumoniae is closely related to C. trachomatis, as the whole genome comparison of at least two isolates from each species has shown [Kalman et al. (1999) Nature Genetics 21:385-389; Read et al. (2000) Nucleic Acids Res 28:1397-406; Stephens et al. (1998) Science 282:754-759]. Based on surface reaction with patient immune sera, the current view is that only one serotype of C. pneumoniae exists world-wide.

C. pneumoniae is a common cause of human respiratory disease. It was first isolated from the conjunctiva of a child in Taiwan in 1965, and was established as a major respiratory pathogen in 1983. In the USA, C. pneumoniae causes approximately 10% of community-acquired pneumonia and 5% of pharyngitis, bronchitis, and sinusitis.

More recently, the spectrum of C. pneumoniae infections has been extended to include atherosclerosis, coronary heart disease, carotid artery stenosis, myocardial infarction, cerebrovascular disease, aortic aneurysm, claudication, and stroke. The association of C. pneumoniae with atherosclerosis is corroborated by the presence of the organism in atherosclerotic lesions throughout the arterial tree and the near absence of the organism in healthy arterial tissue. C. pneumoniae has also been isolated from coronary and carotid atheromatous plaques. The bacterium has also been associated with other acute and chronic respiratory diseases (e.g. otitis media, chronic obstructive pulmonary disease, pulmonary exacerbation of cystic fibrosis) as a result of sero-epidemiologic observations, case reports, isolation or direct detection of the organism in specimens, and successful response to anti-chlamydial antibiotics. To determine whether chronic infection plays a role in initiation or progression of disease, intervention studies in humans have been initiated, and animal models of C. pneumoniae infection have been developed.

Considerable knowledge of the epidemiology of C. pneumoniae infection has been derived from serologic studies using the C. pneumoniae-specific microimmunofluorescence test. Infection is ubiquitous, and it is estimated that virtually everyone is infected at some point in life, with common re-infection. Antibodies against C. pneumoniae are rare in children under the age of 5, except in developing and tropical countries. Antibody prevalence increases rapidly at ages 5 to 14, reaching 50% at the age of 20, and continuing to increase slowly to ˜80% by age 70.

A current hypothesis is that C. pneumoniae can persist in an asymptomatic low-grade infection in very large sections of the human population. When this condition occurs, it believed that the presence of C. pneumoniae, and/or the effects of the host reaction to the bacterium, can cause or help progress of cardiovascular illness.

It is not yet clear whether C. pneumoniae is actually a causative agent of cardiovascular disease, or whether it is just artefactually associated with it. It has been shown, however, that C. pneumoniae infection can induce LDL oxidation by human monocytes [Kalayoglu et al. (1999) J. Infect. Dis. 180:780-90; Kalayoglu et al. (1999) Am. Heart J. 138:S488-490]. As LDL oxidation products are highly atherogenic, this observation provides a possible mechanism whereby C. pneumoniae may cause atheromatous degeneration. If a causative effect is confirmed, vaccination (prophylactic and therapeutic) will be universally recommended.

Genomic sequence information has been published for C. pneumoniae [Kalman et al. (1999) supra; Read et al. (2000) supra; Shirai et al. (2000) J. Infect. Dis. 181(Suppl 3):S524-S527; WO99/27105; WO00/27994] and is available from GenBank. Sequencing efforts have not, however, focused on vaccination, and the availability of genomic sequence does not in itself indicate which of the >1000 genes might encode useful antigens for immunization and vaccination. WO99/27105, for instance, implies that every one of the 1296 ORFs identified in the C. pneumoniae strain CM1 genome is a useful vaccine antigen.

It is thus an object of the present invention to identify antigens useful for vaccine production and development from amongst the many proteins present in C. pneumoniae. It is a further object to identify antigens useful for diagnosis (e.g. immunodiagnosis) of C. pneumoniae.

### DISCLOSURE OF THE INVENTION

The invention provides proteins comprising the C. pneumoniae amino acid sequences disclosed in the examples.

It also provides proteins comprising sequences which share at least x % sequence identity with the C. pneumoniae amino acid sequences disclosed in the examples. Depending on the particular sequence, x is preferably 50% or more (e.g. 60%, 70%, 80%, 90%, 95%, 99% or more). These include mutants and allelic variants. Typically, 50% identity or more between two proteins is considered to be an indication of functional equivalence. Identity between proteins is preferably determined by the Smith-Waterman homology search algorithm as implemented in the MPSRCH program (Oxford Molecular), using an affine gap search with parameters gap open penalty=12 and gap extension penalty=1.

The invention further provides proteins comprising fragments of the C. pneumoniae amino acid sequences disclosed in the examples. The fragments should comprise at least n consecutive amino acids from the sequences and, depending on the particular sequence, n is 7 or more (e.g. 8, 10, 12, 14, 16, 18, 20, 30, 40, 50, 75, 100 or more). Preferably the fragments comprise one or more epitope(s) from the sequence. Other preferred fragments omit a signal peptide.

The proteins of the invention can, of course, be prepared by various means (e.g. native expression, recombinant expression, purification from cell culture, chemical synthesis etc.) and in various forms (e.g. native, fusions etc.). They are preferably prepared in substantially pure form (ie. substantially free from other C. pneumoniae or host cell proteins). Heterologous expression in E. coli is a preferred preparative route.

According to a further aspect, the invention provides nucleic acid comprising the C. pneumoniae nucleotide sequences disclosed in the examples. In addition, the invention provides nucleic acid comprising sequences which share at least x % sequence identity with the C. pneumoniae nucleotide sequences disclosed in the examples. Depending on the particular sequence, x is preferably 50% or more (e.g. 60%, 70%, 80%, 90%, 95%, 99% or more).

Furthermore, the invention provides nucleic acid which can hybridise to the C. pneumoniae nucleic acid disclosed in the examples, preferably under “high stringency” conditions (e.g. 65° C. in a 0.1×SSC, 0.5% SDS solution).

Nucleic acid comprising fragments of these sequences are also provided. These should comprise at least n consecutive nucleotides from the C. pneumoniae sequences and, depending on the particular sequence, n is 10 or more (e.g. 12, 14, 15, 18, 20, 25, 30, 35, 40, 50, 75, 100, 200, 300 or more).

According to a further aspect, the invention provides nucleic acid encoding the proteins and protein fragments of the invention.

It should also be appreciated that the invention provides nucleic acid comprising sequences complementary to those described above (e.g. for antisense or probing purposes).

Nucleic acid according to the invention can, of course, be prepared in many ways (e.g. by chemical synthesis, from genomic or cDNA libraries, from the organism itself etc.) and can take various forms (e.g. single stranded, double stranded, vectors, probes etc.).

In addition, the term “nucleic acid” includes DNA and RNA, and also their analogues, such as those containing modified backbones, and also peptide nucleic acids (PNA) etc.

According to a further aspect, the invention provides vectors comprising nucleotide sequences of the invention (e.g. cloning or expression vectors) and host cells transformed therewith.

According to a further aspect, the invention provides immunogenic compositions comprising protein and/or nucleic acid according to the invention. These compositions are suitable for immunization and vaccination purposes. Vaccines of the invention may be prophylactic or therapeutic, and will typically comprise an antigen which can induce antibodies capable of inhibiting (a) chlamydial adhesion, (b) chlamydial entry, and/or (c) successful replication within the host cell. The vaccines preferably induce any cell-mediated T-cell responses which are necessary for chlamydial clearance from the host.

The invention also provides nucleic acid or protein according to the invention for use as medicaments (e.g. as vaccines). It also provides the use of nucleic acid or protein according to the invention in the manufacture of a medicament (e.g. a vaccine or an immunogenic composition) for treating or preventing infection due to C. pneumoniae.

The invention also provides a method of treating (e.g. immunizing) a patient, comprising administering to the patient a therapeutically effective amount of nucleic acid or protein according to the invention.

According to further aspects, the invention provides various processes.

A process for producing proteins of the invention is provided, comprising the step of culturing a host cell according to the invention under conditions which induce protein expression.

A process for producing protein or nucleic acid of the invention is provided, wherein the protein or nucleic acid is synthesised in part or in whole using chemical means.

A process for detecting C. pneumoniae in a sample is provided, wherein the sample is contacted with an antibody which binds to a protein of the invention.

A summary of standard techniques and procedures which may be employed in order to perform the invention (e.g. to utilise the disclosed sequences for immunization) follows. This summary is not a limitation on the invention but, rather, gives examples that may be used, but are not required.

General

The practice of the present invention will employ, unless otherwise indicated, conventional techniques of molecular biology, microbiology, recombinant DNA, and immunology, which are within the skill of the art. Such techniques are explained fully in the literature e.g. Sambrook Molecular Cloning; A Laboratory Manual, Second Edition (1989) and Third Edition (2001); DNA Cloning, Volumes I and ii (D. N Glover ed. 1985); Oligonucleotide Synthesis (M. J. Gait ed, 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Mayer and Walker, eds. (1987), Immunochemical Methods in Cell and Molecular Biology (Academic Press, London); Scopes, (1987) Protein Purification: Principles and Practice, Second Edition (Springer-Verlag, N.Y.), and Handbook of Experimental Immunology, Volumes I-IV (D. M. Weir and C. C. Blackwell eds 1986).

Standard abbreviations for nucleotides and amino acids are used in this specification.

Definitions

A composition containing X is “substantially free of” Y when at least 85% by weight of the total X+Y in the composition is X. Preferably, X comprises at least about 90% by weight of the total of X+Y in the composition, more preferably at least about 95% or even 99% by weight.

The term “comprising” means “including” as well as “consisting” e.g. a composition “comprising” X may consist exclusively of X or may include something additional to X, such as X+Y.

The term “heterologous” refers to two biological components that are not found together in nature. The components may be host cells, genes, or regulatory regions, such as promoters. Although the heterologous components are not found together in nature, they can function together, as when a promoter heterologous to a gene is operably linked to the gene. Another example is where a Chlamydial sequence is heterologous to a mouse host cell. A further examples would be two epitopes from the same or different proteins which have been assembled in a single protein in an arrangement not found in nature.

An “origin of replication” is a polynucleotide sequence that initiates and regulates replication of polynucleotides, such as an expression vector. The origin of replication behaves as an autonomous unit of polynucleotide replication within a cell, capable of replication under its own control. An origin of replication may be needed for a vector to replicate in a particular host cell. With certain origins of replication, an expression vector can be reproduced at a high copy number in the presence of the appropriate proteins within the cell. Examples of origins are the autonomously replicating sequences, which are effective in yeast; and the viral T-antigen, effective in COS-7 cells.

A “mutant” sequence is defined as DNA, RNA or amino acid sequence differing from but having sequence identity with the native or disclosed sequence. Depending on the particular sequence, the degree of sequence identity between the native or disclosed sequence and the mutant sequence is preferably greater than 50% (e.g. 60%, 70%, 80%, 90%, 95%, 99% or more, calculated using the Smith-Waterman algorithm as described above). As used herein, an “allelic variant” of a nucleic acid molecule, or region, for which nucleic acid sequence is provided herein is a nucleic acid molecule, or region, that occurs essentially at the same locus in the genome of another or second isolate, and that, due to natural variation caused by, for example, mutation or recombination, has a similar but not identical nucleic acid sequence. A coding region allelic variant typically encodes a protein having similar activity to that of the protein encoded by the gene to which it is being compared. An allelic variant can also comprise an alteration in the 5′ or 3′ untranslated regions of the gene, such as in regulatory control regions (e.g. see U.S. Pat. No. 5,753,235).

Expression Systems

The Chlamydial nucleotide sequences can be expressed in a variety of different expression systems; for example those used with mammalian cells, baculoviruses, plants, bacteria, and yeast.

i. Mammalian Systems

Mammalian expression systems are known in the art. A mammalian promoter is any DNA sequence capable of binding mammalian RNA polymerase and initiating the downstream (3′) transcription of a coding sequence (e.g. structural gene) into mRNA. A promoter will have a transcription initiating region, which is usually placed proximal to the 5′ end of the coding sequence, and a TATA box, usually located 25-30 base pairs (bp) upstream of the transcription initiation site. The TATA box is thought to direct RNA polymerase II to begin RNA synthesis at the correct site. A mammalian promoter will also contain an upstream promoter element, usually located within 100 to 200 bp upstream of the TATA box. An upstream promoter element determines the rate at which transcription is initiated and can act in either orientation [Sambrook et al. (1989) “Expression of Cloned Genes in Mammalian Cells.” In Molecular Cloning: A Laboratory Manual, 2nd ed].

Mammalian viral genes are often highly expressed and have a broad host range; therefore sequences encoding mammalian viral genes provide particularly useful promoter sequences. Examples include the SV40 early promoter, mouse mammary tumor virus LTR promoter, adenovirus major late promoter (Ad MLP), and herpes simplex virus promoter. In addition, sequences derived from non-viral genes, such as the murine metallotheionein gene, also provide useful promoter sequences. Expression may be either constitutive or regulated (inducible), depending on the promoter can be induced with glucocorticoid in hormone-responsive cells.

The presence of an enhancer element (enhancer), combined with the promoter elements described above, will usually increase expression levels. An enhancer is a regulatory DNA sequence that can stimulate transcription up to 1000-fold when linked to homologous or heterologous promoters, with synthesis beginning at the normal RNA start site. Enhancers are also active when they are placed upstream or downstream from the transcription initiation site, in either normal or flipped orientation, or at a distance of more than 1000 nucleotides from the promoter [Maniatis et al. (1987) Science 236:1237; Alberts et al. (1989) Molecular Biology of the Cell, 2nd ed.]. Enhancer elements derived from viruses may be particularly useful, because they usually have a broader host range. Examples include the SV40 early gene enhancer [Dijkema et al (1985) EMBO J. 4:761] and the enhancer/promoters derived from the long terminal repeat (LTR) of the Rous Sarcoma Virus [Gorman et al. (1982) PNAS USA 79:6777] and from human cytomegalovirus [Boshart et al. (1985) Cell 41:521]. Additionally, some enhancers are regulatable and become active only in the presence of an inducer, such as a hormone or metal ion [Sassone-Corsi and Borelli (1986) Trends Genet. 2:215; Maniatis et al. (1987) Science 236:1237].

A DNA molecule may be expressed intracellularly in mammalian cells. A promoter sequence may be directly linked with the DNA molecule, in which case the first amino acid at the N-terminus of the recombinant protein will always be a methionine, which is encoded by the ATG start codon. If desired, the N-terminus may be cleaved from the protein by in vitro incubation with cyanogen bromide.

Alternatively, foreign proteins can also be secreted from the cell into the growth media by creating chimeric DNA molecules that encode a fusion protein comprised of a leader sequence fragment that provides for secretion of the foreign protein in mammalian cells. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell. The adenovirus triparite leader is an example of a leader sequence that provides for secretion of a foreign protein in mammalian cells.

Usually, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-transcriptional cleavage and polyadenylation [Birnstiel et al. (1985) Cell 41:349; Proudfoot and Whitelaw (1988) “Termination and 3′ end processing of eukaryotic RNA. In Transcription and splicing (ed. B. D. Hames and D. M. Glover); Proudfoot (1989) Trends Biochem. Sci. 14:105]. These sequences direct the transcription of an mRNA which can be translated into the polypeptide encoded by the DNA. Examples of transcription terminater/polyadenylation signals include those derived from SV40 [Sambrook et al (1989) “Expression of cloned genes in cultured mammalian cells.” In Molecular Cloning: A Laboratory Manual].

Usually, the above described components, comprising a promoter, polyadenylation signal, and transcription termination sequence are put together into expression constructs. Enhancers, introns with functional splice donor and acceptor sites, and leader sequences may also be included in an expression construct, if desired. Expression constructs are often maintained in a replicon, such as an extrachromosomal element (e.g. plasmids) capable of stable maintenance in a host, such as mammalian cells or bacteria. Mammalian replication systems include those derived from animal viruses, which require trans-acting factors to replicate. For example, plasmids containing the replication systems of papovaviruses, such as SV40 [Gluzman (1981) Cell 23:175] or polyomavirus, replicate to extremely high copy number in the presence of the appropriate viral T antigen. Additional examples of mammalian replicons include those derived from bovine papillomavirus and Epstein-Barr virus. Additionally, the replicon may have two replicaton systems, thus allowing it to be maintained, for example, in mammalian cells for expression and in a prokaryotic host for cloning and amplification. Examples of such mammalian-bacteria shuttle vectors include pMT2 [Kaufman et al. (1989) Mol. Cell. Biol. 9:946] and pHEBO [Shimizu et al. (1986) Mol. Cell. Biol. 6:1074].

The transformation procedure used depends upon the host to be transformed. Methods for introduction of heterologous polynucleotides into mammalian cells are known in the art and include dextran-mediated transfection, calcium phosphate precipitation, polybrene-mediated transfection, protoplast fusion, electroporation, encapsulation of polynucleotide(s) in liposomes, direct microinjection of the DNA into nuclei.

Mammalian cell lines available as hosts for expression are known in the art and include many immortalized cell lines available from the American Type Culture Collection (ATCC), including but not limited to, Chinese hamster ovary (CHO) cells, HeLa cells, baby hamster kidney (BHK) cells, monkey kidney cells (COS), human hepatocellular carcinoma cells (e.g. Hep G2), and a number of other cell lines.

ii. Baculovirus Systems

The polynucleotide encoding the protein can also be inserted into a suitable insect expression vector, and is operably linked to the control elements within that vector. Vector construction employs techniques which are known in the art. Generally, the components of the expression system include a transfer vector, usually a bacterial plasmid, which contains both a fragment of the baculovirus genome, and a convenient restriction site for insertion of the heterologous gene or genes to be expressed; a wild type baculovirus with a sequence homologous to the baculovirus-specific fragment in the transfer vector (this allows for the homologous recombination of the heterologous gene in to the baculovirus genome); and appropriate insect host cells and growth media.

After inserting the DNA sequence encoding the protein into the transfer vector, the vector and the wild type viral genome are transfected into an insect host cell where the vector and viral genome are allowed to recombine. The packaged recombinant virus is expressed and recombinant plaques are identified and purified. Materials and methods for baculovirus/insect cell expression systems are commercially available in kit form from, inter alia, Invitrogen, San Diego Calif. (“MaxBac” kit). These techniques are generally known to those skilled in the art and fully described in Summers and Smith, Texas Agricultural Experiment Station Bulletin No. 1555 (1987) (hereinafter “Summers and Smith”).

Prior to inserting the DNA sequence encoding the protein into the baculovirus genome, the above described components, comprising a promoter, leader (if desired), coding sequence of interest, and transcription termination sequence, are usually assembled into an intermediate transplacement construct (transfer vector). This construct may contain a single gene and operably linked regulatory elements; multiple genes, each with its owned set of operably linked regulatory elements; or multiple genes, regulated by the same set of regulatory elements. Intermediate transplacement constructs are often maintained in a replicon, such as an extrachromosomal element (e.g. plasmids) capable of stable maintenance in a host, such as a bacterium. The replicon will have a replication system, thus allowing it to be maintained in a suitable host for cloning and amplification.

Currently, the most commonly used transfer vector for introducing foreign genes into AcNPV is pAc373. Many other vectors, known to those of skill in the art, have also been designed. These include, for example, pVL985 (which alters the polyhedrin start codon from ATG to ATT, and which introduces a BamHI cloning site 32 basepairs downstream from the ATT; see Luckow and Summers, Virology (1989) 17:31.

The plasmid usually also contains the polyhedrin polyadenylation signal (Miller et al. (1988) Ann. Rev. Microbiol., 42:177) and a prokaryotic ampicillin-resistance (amp) gene and origin of replication for selection and propagation in E. coli.

Baculovirus transfer vectors usually contain a baculovirus promoter. A baculovirus promoter is any DNA sequence capable of binding a baculovirus RNA polymerase and initiating the downstream (5′ to 3′) transcription of a coding sequence (e.g. structural gene) into mRNA. A promoter will have a transcription initiation region which is usually placed proximal to the 5′ end of the coding sequence. This transcription initiation region usually includes an RNA polymerase binding site and a transcription initiation site. A baculovirus transfer vector may also have a second domain called an enhancer, which, if present, is usually distal to the structural gene. Expression may be either regulated or constitutive.

Structural genes, abundantly transcribed at late times in a viral infection cycle, provide particularly useful promoter sequences. Examples include sequences derived from the gene encoding the viral polyhedron protein, Friesen et al., (1986) “The Regulation of Baculovirus Gene Expression,” in: The Molecular Biology of Baculoviruses (ed. Walter Doerfler); EPO Publ. Nos. 127 839 and 155 476; and the gene encoding the p10 protein, Vlak et al., (1988), J. Gen. Virol. 69:765.

DNA encoding suitable signal sequences can be derived from genes for secreted insect or baculovirus proteins, such as the baculovirus polyhedrin gene (Carbonell et al. (1988) Gene, 73:409). Alternatively, since the signals for mammalian cell posttranslational modifications (such as signal peptide cleavage, proteolytic cleavage, and phosphorylation) appear to be recognized by insect cells, and the signals required for secretion and nuclear accumulation also appear to be conserved between the invertebrate cells and vertebrate cells, leaders of non-insect origin, such as those derived from genes encoding human α-interferon, Maeda et al., (1985), Nature 315:592; human gastrin-releasing peptide, Lebacq-Verheyden et al., (1988), Molec. Cell. Biol. 8:3129; human IL-2, Smith et al., (1985) Proc. Nat'l Acad. Sci. USA, 82:8404; mouse IL-3, (Miyajima et al., (1987) Gene 58:273; and human glucocerebrosidase, Martin et al. (1988) DNA, 7:99, can also be used to provide for secretion in insects.

A recombinant polypeptide or polyprotein may be expressed intracellularly or, if it is expressed with the proper regulatory sequences, it can be secreted. Good intracellular expression of nonfused foreign proteins usually requires heterologous genes that ideally have a short leader sequence containing suitable translation initiation signals preceding an ATG start signal. If desired, methionine at the N-terminus may be cleaved from the mature protein by in vitro incubation with cyanogen bromide.

Alternatively, recombinant polyproteins or proteins which are not naturally secreted can be secreted from the insect cell by creating chimeric DNA molecules that encode a fusion protein comprised of a leader sequence fragment that provides for secretion of the foreign protein in insects. The leader sequence fragment usually encodes a signal peptide comprised of hydrophobic amino acids which direct the translocation of the protein into the endoplasmic reticulum.

After insertion of the DNA sequence and/or the gene encoding the expression product precursor of the protein, an insect cell host is co-transformed with the heterologous DNA of the transfer vector and the genomic DNA of wild type baculovirus—usually by co-transfection. The promoter and transcription termination sequence of the construct will usually comprise a 2-5 kb section of the baculovirus genome. Methods for introducing heterologous DNA into the desired site in the baculovirus virus are known in the art. (See Summers and Smith supra; Ju et al. (1987); Smith et al., Mol. Cell. Biol. (1983) 3:2156; and Luckow and Summers (1989)). For example, the insertion can be into a gene such as the polyhedrin gene, by homologous double crossover recombination; insertion can also be into a restriction enzyme site engineered into the desired baculovirus gene. Miller et al., (1989), Bioessays 4:91. The DNA sequence, when cloned in place of the polyhedrin gene in the expression vector, is flanked both 5′ and 3′ by polyhedrin-specific sequences and is positioned downstream of the polyhedrin promoter.

The newly formed baculovirus expression vector is subsequently packaged into an infectious recombinant baculovirus. Homologous recombination occurs at low frequency (between ˜1% and ˜5%); thus, the majority of the virus produced after cotransfection is still wild-type virus. Therefore, a method is necessary to identify recombinant viruses. An advantage of the expression system is a visual screen allowing recombinant viruses to be distinguished. The polyhedrin protein, which is produced by the native virus, is produced at very high levels in the nuclei of infected cells at late times after viral infection. Accumulated polyhedrin protein forms occlusion bodies that also contain embedded particles. These occlusion bodies, up to 15 μm in size, are highly refractile, giving them a bright shiny appearance that is readily visualized under the light microscope. Cells infected with recombinant viruses lack occlusion bodies. To distinguish recombinant virus from wild-type virus, the transfection supernatant is plaqued onto a monolayer of insect cells by techniques known to those skilled in the art. Namely, the plaques are screened under the light microscope for the presence (indicative of wild-type virus) or absence (indicative of recombinant virus) of occlusion bodies. “Current Protocols in Microbiology” Vol. 2 (Ausubel et al. eds) at 16.8 (Supp. 10, 1990); Summers & Smith, supra; Miller et al. (1989).

Recombinant baculovirus expression vectors have been developed for infection into several insect cells. For example, recombinant baculoviruses have been developed for, inter alia: Aedes aegypti, Autographa californica, Bombyx mori, Drosophila melanogaster, Spodoptera frugiperda, and Trichoplusia ni (WO 89/046699; Carbonell et al., (1985) J. Virol. 56:153; Wright (1986) Nature 321:718; Smith et al., (1983) Mol. Cell. Biol. 3:2156; and see generally, Fraser, et al. (1989) In Vitro Cell. Dev. Biol. 25:225).

Cells and cell culture media are commercially available for both direct and fusion expression of heterologous polypeptides in a baculovirus/expression system; cell culture technology is generally known to those skilled in the art. See, e.g. Summers and Smith supra.

The modified insect cells may then be grown in an appropriate nutrient medium, which allows for stable maintenance of the plasmid(s) present in the modified insect host. Where the expression product gene is under inducible control, the host may be grown to high density, and expression induced. Alternatively, where expression is constitutive, the product will be continuously expressed into the medium and the nutrient medium must be continuously circulated, while removing the product of interest and augmenting depleted nutrients. The product may be purified by such techniques as chromatography, e.g. HPLC, affinity chromatography, ion exchange chromatography, etc.; electrophoresis; density gradient centrifugation; solvent extraction, or the like. As appropriate, the product may be further purified, as required, so as to remove substantially any insect proteins which are also secreted in the medium or result from lysis of insect cells, so as to provide a product which is at least substantially free of host debris, e.g. proteins, lipids and polysaccharides.

In order to obtain protein expression, recombinant host cells derived from the transformants are incubated under conditions which allow expression of the recombinant protein encoding sequence. These conditions will vary, dependent upon the host cell selected. However, the conditions are readily ascertainable to those of ordinary skill in the art, based upon what is known in the art.

iii. Plant Systems

There are many plant cell culture and whole plant genetic expression systems known in the art. Exemplary plant cellular genetic expression systems include those described in patents, such as: U.S. Pat. No. 5,693,506; U.S. Pat. No. 5,659,122; and U.S. Pat. No. 5,608,143. Additional examples of genetic expression in plant cell culture has been described by Zenk, Phytochemistry 30:3861-3863 (1991). Descriptions of plant protein signal peptides may be found in addition to the references described above in Vaulcombe et al., Mol. Gen. Genet. 209:33-40 (1987); Chandler et al., Plant Molecular Biology 3:407-418 (1984); Rogers, J. Biol. Chem. 260:3731-3738 (1985); Rothstein et al., Gene 55:353-356 (1987); Whittier et al., Nucleic Acids Research 15:2515-2535 (1987); Wirsel et al., Molecular Microbiology 3:3-14 (1989); Yu et al., Gene 122:247-253 (1992). A description of the regulation of plant gene expression by the phytohormone, gibberellic acid and secreted enzymes induced by gibberellic acid can be found in R. L. Jones and J. MacMillin, Gibberellins: in: Advanced Plant Physiology,. Malcolm B. Wilkins, ed., 1984 Pitman Publishing Limited, London, pp. 21-52. References that describe other metabolically-regulated genes: Sheen, Plant Cell, 2:1027-1038(1990); Maas et al., EMBO J. 9:3447-3452 (1990); Benkel and Hickey, Proc. Natl. Acad. Sci. 84:1337-1339 (1987)

Typically, using techniques known in the art, a desired polynucleotide sequence is inserted into an expression cassette comprising genetic regulatory elements designed for operation in plants. The expression cassette is inserted into a desired expression vector with companion sequences upstream and downstream from the expression cassette suitable for expression in a plant host. The companion sequences will be of plasmid or viral origin and provide necessary characteristics to the vector to permit the vectors to move DNA from an original cloning host, such as bacteria, to the desired plant host. The basic bacterial/plant vector construct will preferably provide a broad host range prokaryote replication origin; a prokaryote selectable marker; and, for Agrobacterium transformations, T DNA sequences for Agrobacterium-mediated transfer to plant chromosomes. Where the heterologous gene is not readily amenable to detection, the construct will preferably also have a selectable marker gene suitable for determining if a plant cell has been transformed. A general review of suitable markers, for example for the members of the grass family, is found in Wilmink and Dons, 1993, Plant Mol. Biol. Reptr, 11(2):165-185.

Sequences suitable for permitting integration of the heterologous sequence into the plant genome are also recommended. These might include transposon sequences and the like for homologous recombination as well as Ti sequences which permit random insertion of a heterologous expression cassette into a plant genome. Suitable prokaryote selectable markers include resistance toward antibiotics such as ampicillin or tetracycline. Other DNA sequences encoding additional functions may also be present in the vector, as is known in the art.

The nucleic acid molecules of the subject invention may be included into an expression cassette for expression of the protein(s) of interest. Usually, there will be only one expression cassette, although two or more are feasible. The recombinant expression cassette will contain in addition to the heterologous protein encoding sequence the following elements, a promoter region, plant 5′ untranslated sequences, initiation codon depending upon whether or not the structural gene comes equipped with one, and a transcription and translation termination sequence. Unique restriction enzyme sites at the 5′ and 3′ ends of the cassette allow for easy insertion into a pre-existing vector.

A heterologous coding sequence may be for any protein relating to the present invention. The sequence encoding the protein of interest will encode a signal peptide which allows processing and translocation of the protein, as appropriate, and will usually lack any sequence which might result in the binding of the desired protein of the invention to a membrane. Since, for the most part, the transcriptional initiation region will be for a gene which is expressed and translocated during germination, by employing the signal peptide which provides for translocation, one may also provide for translocation of the protein of interest. In this way, the protein(s) of interest will be translocated from the cells in which they are expressed and may be efficiently harvested.

Typically secretion in seeds are across the aleurone or scutellar epithelium layer into the endosperm of the seed. While it is not required that the protein be secreted from the cells in which the protein is produced, this facilitates the isolation and purification of the recombinant protein.

Since the ultimate expression of the desired gene product will be in a eucaryotic cell it is desirable to determine whether any portion of the cloned gene contains sequences which will be processed out as introns by the host's splicosome machinery. If so, site-directed mutagenesis of the “intron” region may be conducted to prevent losing a portion of the genetic message as a false intron code, Reed and Maniatis, Cell 41:95-105, 1985.

The vector can be microinjected directly into plant cells by use of micropipettes to mechanically transfer the recombinant DNA. Crossway, Mol. Gen. Genet, 202:179-185, 1985. The genetic material may also be transferred into the plant cell by using polyethylene glycol, Krens, et al., Nature, 296, 72-74, 1982. Another method of introduction of nucleic acid segments is high velocity ballistic penetration by small particles with the nucleic acid either within the matrix of small beads or particles, or on the surface, Klein, et al., Nature, 327, 70-73, 1987 and Knudsen and Muller, 1991, Planta, 185:330-336 teaching particle bombardment of barley endosperm to create transgenic barley. Yet another method of introduction would be fusion of protoplasts with other entities, either minicells, cells, lysosomes or other fusible lipid-surfaced bodies, Fraley, et al., Proc. Natl. Acad. Sci. USA, 79, 1859-1863, 1982.

The vector may also be introduced into the plant cells by electroporation. (Fromm et al., Proc. Natl Acad. Sci. USA 82:5824, 1985). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

All plants from which protoplasts can be isolated and cultured to give whole regenerated plants can be transformed by the present invention so that whole plants are recovered which contain the transferred gene. It is known that practically all plants can be regenerated from cultured cells or tissues, including but not limited to all major species of sugarcane, sugar beet, cotton, fruit and other trees, legumes and vegetables. Some suitable plants include, for example, species from the genera Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersion, Nicotiana, Solanum, Petunia, Digitalis, Majorana, Cichorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, and Datura.

Means for regeneration vary from species to species of plants, but generally a suspension of transformed protoplasts containing copies of the heterologous gene is first provided. Callus tissue is formed and shoots may be induced from callus and subsequently rooted. Alternatively, embryo formation can be induced from the protoplast suspension. These embryos germinate as natural embryos to form plants. The culture media will generally contain various amino acids and hormones, such as auxin and cytokinins. It is also advantageous to add glutamic acid and proline to the medium, especially for such species as corn and alfalfa. Shoots and roots normally develop simultaneously. Efficient regeneration will depend on the medium, on the genotype, and on the history of the culture. If these three variables are controlled, then regeneration is fully reproducible and repeatable.

In some plant cell culture systems, the desired protein of the invention may be excreted or alternatively, the protein may be extracted from the whole plant. Where the desired protein of the invention is secreted into the medium, it may be collected. Alternatively, the embryos and embryoless-half seeds or other plant tissue may be mechanically disrupted to release any secreted protein between cells and tissues. The mixture may be suspended in a buffer solution to retrieve soluble proteins. Conventional protein isolation and purification methods will be then used to purify the recombinant protein. Parameters of time, temperature pH, oxygen, and volumes will be adjusted through routine methods to optimize expression and recovery of heterologous protein.

iv. Bacterial Systems

Bacterial expression techniques are known in the art. A bacterial promoter is any DNA sequence capable of binding bacterial RNA polymerase and initiating the downstream (3′) transcription of a coding sequence (e.g. structural gene) into mRNA. A promoter will have a transcription initiation region which is usually placed proximal to the 5′ end of the coding sequence. This transcription initiation region usually includes an RNA polymerase binding site and a transcription initiation site. A bacterial promoter may also have a second domain called an operator, that may overlap an adjacent RNA polymerase binding site at which RNA synthesis begins. The operator permits negative regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence. An example of a gene activator protein is the catabolite activator protein (CAP), which helps initiate transcription of the lac operon in Escherichia coli (E. coli) [Raibaud et al. (1984) Annu. Rev. Genet. 18:173]. Regulated expression may therefore be either positive or negative, thereby either enhancing or reducing transcription.

Sequences encoding metabolic pathway enzymes provide particularly useful promoter sequences. Examples include promoter sequences derived from sugar metabolizing enzymes, such as galactose, lactose (lac) [Chang et al. (1977) Nature 198:1056], and maltose. Additional examples include promoter sequences derived from biosynthetic enzymes such as tryptophan (trp) [Goeddel et al. (1980) Nuc. Acids Res. 8:4057; Yelverton et al. (1981) Nucl. Acids Res. 9:731; U.S. Pat. No. 4,738,921; EP-A-0036776 and EP-A-0121775]. The g-laotamase (bla) promoter system [Weissmann (1981) “The cloning of interferon and other mistakes.” In Interferon 3 (ed. I. Gresser)], bacteriophage lambda PL [Shimatake et al. (1981) Nature 292:128] and T5 [U.S. Pat. No. 4,689,406] promoter systems also provide useful promoter sequences.

In addition, synthetic promoters which do not occur in nature also function as bacterial promoters. For example, transcription activation sequences of one bacterial or bacteriophage promoter may be joined with the operon sequences of another bacterial or bacteriophage promoter, creating a synthetic hybrid promoter [U.S. Pat. No. 4,551,433]. For example, the tac promoter is a hybrid trp-lac promoter comprised of both trp promoter and lac operon sequences that is regulated by the lac repressor [Amann et al. (1983) Gene 25:167; de Boer et al. (1983) Proc. Natl. Acad. Sci. 80:21]. Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The bacteriophage T7 RNA polymerase/promoter system is an example of a coupled promoter system [Studier et al. (1986) J. Mol. Biol. 189:113; Tabor et al. (1985) Proc Natl. Acad. Sci. 82:1074]. In addition, a hybrid promoter can also be comprised of a bacteriophage promoter and an E. coli operator region (EPO-A-0 267 85 1).

In addition to a functioning promoter sequence, an efficient ribosome binding site is also useful for the expression of foreign genes in prokaryotes. In E. coli, the ribosome binding site is called the Shine-Dalgarno (SD) sequence and includes an initiation codon (ATG) and a sequence 3-9 nucleotides in length located 3-11 nucleotides upstream of the initiation codon [Shine et al. (1975) Nature 254:34]. The SD sequence is thought to promote binding of mRNA to the ribosome by the pairing of bases between the SD sequence and the 3′ and of E. coli 16S rRNA [Steitz et al. (1979) “Genetic signals and nucleotide sequences in messenger RNA.” In Biological Regulation and Development: Gene Expression (ed. R. F. Goldberger)]. To express eukaryotic genes and prokaryotic genes with weak ribosome-binding site [Sambrook et al. (1989) “Expression of cloned genes in Escherichia coli.” In Molecular Cloning: A Laboratory Manual].

A DNA molecule may be expressed intracellularly. A promoter sequence may be directly linked with the DNA molecule, in which case the first amino acid at the N-terminus will always be a methionine, which is encoded by the ATG start codon. If desired, methionine at the N-terminus may be cleaved from the protein by in vitro incubation with cyanogen bromide or by either in vivo on in vitro incubation with a bacterial methionine N-terminal peptidase (EPO-A-0 219 237).

Fusion proteins provide an alternative to direct expression. Usually, a DNA sequence encoding the N-terminal portion of an endogenous bacterial protein, or other stable protein, is fused to the 5′ end of heterologous coding sequences. Upon expression, this construct will provide a fusion of the two amino acid sequences. For example, the bacteriophage lambda cell gene can be linked at the 5′ terminus of a foreign gene and expressed in bacteria. The resulting fusion protein preferably retains a site for a processing enzyme (factor Xa) to cleave the bacteriophage protein from the foreign gene [Nagai et al. (1984) Nature 309:810]. Fusion proteins can also be made with sequences from the lacZ [Jia et al. (1987) Gene 60:197], trpE [Allen et al. (1987) J. Biotechnol. 5:93; Makoff et al. (1989) J. Gen. Microbiol. 135:11], and Chey [EP-A-0 324 647] genes. The DNA sequence at the junction of the two amino acid sequences may or may not encode a cleavable site. Another example is a ubiquitin fusion protein. Such a fusion protein is made with the ubiquitin region that preferably retains a site for a processing enzyme (e.g. ubiquitin specific processing-protease) to cleave the ubiquitin from the foreign protein. Through this method, native foreign protein can be isolated [Miller et al. (1989) Bio/Technology 7:698].

Alternatively, foreign proteins can also be secreted from the cell by creating chimeric DNA molecules that encode a fusion protein comprised of a signal peptide sequence fragment that provides for secretion of the foreign protein in bacteria [U.S. Pat. No. 4,336,336]. The signal sequence fragment usually encodes a signal peptide comprised of hydrophobic amino acids which direct the secretion of the protein from the cell. The protein is either secreted into the growth media (gram-positive bacteria) or into the periplasmic space, located between the inner and outer membrane of the cell (gram-negative bacteria). Preferably there are processing sites, which can be cleaved either in vivo or in vitro encoded between the signal peptide fragment and the foreign gene.

DNA encoding suitable signal sequences can be derived from genes for secreted bacterial proteins, such as the E. coli outer membrane protein gene (ompA) [Masui et al. (1983), in: Experimental Manipulation of Gene Expression; Ghrayeb et al. (1984) EMBO J. 3:2437] and the E. coli alkaline phosphatase signal sequence (phoA) [Oka et al. (1985) Proc. Natl. Acad. Sci. 82:7212]. As an additional example, the signal sequence of the alpha-amylase gene from various Bacillus strains can be used to secrete heterologous proteins from B. subtilis [Palva et al. (1982) Proc. Natl. Acad. Sci. USA 79:5582; EP-A-0 244 042].

Usually, transcription termination sequences recognized by bacteria are regulatory regions located 3′ to the translation stop codon, and thus together with the promoter flank the coding sequence. These sequences direct the transcription of an mRNA which can be translated into the polypeptide encoded by the DNA. Transcription termination sequences frequently include DNA sequences of about 50 nucleotides capable of forming stem loop structures that aid in terminating transcription. Examples include transcription termination sequences derived from genes with strong promoters, such as the trp gene in E. coli as well as other biosynthetic genes.

Usually, the above described components, comprising a promoter, signal sequence (if desired), coding sequence of interest, and transcription termination sequence, are put together into expression constructs. Expression constructs are often maintained in a replicon, such as an extrachromosomal element (e.g. plasmids) capable of stable maintenance in a host, such as bacteria. The replicon will have a replication system, thus allowing it to be maintained in a prokaryotic host either for expression or for cloning and amplification. In addition, a replicon may be either a high or low copy number plasmid. A high copy number plasmid will generally have a copy number ranging from about 5 to about 200, and usually about 10 to about 150. A host containing a high copy number plasmid will preferably contain at least about 10, and more preferably at least about 20 plasmids. Either a high or low copy number vector may be selected, depending upon the effect of the vector and the foreign protein on the host.

Alternatively, the expression constructs can be integrated into the bacterial genome with an integrating vector. Integrating vectors usually contain at least one sequence homologous to the bacterial chromosome that allows the vector to integrate. Integrations appear to result from recombinations between homologous DNA in the vector and the bacterial chromosome. For example, integrating vectors constructed with DNA from various Bacillus strains integrate into the Bacillus chromosome (EP-A-0 127 328). Integrating vectors may also be comprised of bacteriophage or transposon sequences.

Usually, extrachromosomal and integrating expression constructs may contain selectable markers to allow for the selection of bacterial strains that have been transformed. Selectable markers can be expressed in the bacterial host and may include genes which render bacteria resistant to drugs such as ampicillin, chloramphenicol, erythromycin, kanamycin (neomycin), and tetracycline [Davies et al. (1978) Annu. Rev. Microbiol. 32:469]. Selectable markers may also include biosynthetic genes, such as those in the histidine, tryptophan, and leucine biosynthetic pathways.

Alternatively, some of the above described components can be put together in transformation vectors. Transformation vectors are usually comprised of a selectable market that is either maintained in a replicon or developed into an integrating vector, as described above.

Expression and transformation vectors, either extra-chromosomal replicons or integrating vectors, have been developed for transformation into many bacteria. For example, expression vectors have been developed for, inter alia, the following bacteria: Bacillus subtilis [Palva et al. (1982) Proc. Natl. Acad. Sci. USA 79:5582; EP-A-0 036 259 and EP-A-0 063 953; WO 84/04541], Escherichia coli [Shimatake et al. (1981) Nature 292:128; Amann et al. (1985) Gene 40:183; Studier et al. (1986) J. Mol. Biol. 189:113; EP-A-0 036 776, EP-A-0 136 829 and EP-A-0 136 907], Streptococcus cremoris [Powell et al. (1988) Appl. Environ. Microbiol. 54:655]; Streptococcus lividans [Powell et al. (1988) Appl. Environ. Microbiol. 54:655], Streptomyces lividans [U.S. Pat. No. 4,745,056].

Methods of introducing exogenous DNA into bacterial hosts are well-known in the art, and usually include either the transformation of bacteria treated with CaCl₂ or other agents, such as divalent cations and DMSO. DNA can also be introduced into bacterial cells by electroporation. Transformation procedures usually vary with the bacterial species to be transformed. See e.g. [Masson et al. (1989) FEMS Microbiol. Lett. 60:273; Palva et al. (1982) Proc. Natl. Acad. Sci. USA 79:5582; EP-A-0 036 259 and EP-A-0 063 953; WO 84/04541, Bacillus], [Miller et al. (1988) Proc. Natl. Acad. Sci. 85:856; Wang et al. (1990) J. Bacteriol. 172:949, Campylobacter], [Cohen et al. (1973) Proc. Natl. Acad. Sci. 69:2110; Dower et al. (1988) Nucleic Acids Res. 16:6127; Kushner (1978) “An improved method for transformation of Escherichia coli with ColEl-derived plasmids. In Genetic Engineering: Proceedings of the International Symposium on Genetic Engineering (eds. H. W. Boyer and S. Nicosia); Mandel et al. (1970) J. Mol. Biol. 53:159; Taketo (1988) Biochim. Biophys. Acta 949:318; Escherichia], [Chassy et al. (1987) FEMS Microbiol. Lett. 44:173 Lactobacillus]; [Fiedler et al. (1988) Anal. Biochem 170:38, Pseudomonas]; [Augustin et al. (1990) FEMS Microbiol. Lett. 66:203, Staphylococcus], [Barany et al. (1980) J. Bacteriol. 144:698; Harlander (1987) “Transformation of Streptococcus lactis by electroporation, in: Streptococcal Genetics (ed. J. Ferretti and R. Curtiss III); Perry et al. (1981) Infect. Immun. 32:1295; Powell et al. (1988) Appl. Environ. Microbiol. 54:655; Somkuti et al. (1987) Proc. 4th Evr. Cong. Biotechnology 1:412, Streptococcus].

v. Yeast Expression

Yeast expression systems are also known to one of ordinary skill in the art. A yeast promoter is any DNA sequence capable of binding yeast RNA polymerase and initiating the downstream (3′) transcription of a coding sequence (e.g. structural gene) into mRNA. A promoter will have a transcription initiation region which is usually placed proximal to the 5′ end of the coding sequence. This transcription initiation region usually includes an RNA polymerase binding site (the “TATA Box”) and a transcription initiation site. A yeast promoter may also have a second domain called an upstream activator sequence (UAS), which, if present, is usually distal to the structural gene. The UAS permits regulated (inducible) expression. Constitutive expression occurs in the absence of a UAS. Regulated expression may be either positive or negative, thereby either enhancing or reducing transcription.

Yeast is a fermenting organism with an active metabolic pathway, therefore sequences encoding enzymes in the metabolic pathway provide particularly useful promoter sequences. Examples include alcohol dehydrogenase (ADH) (EP-A-0 284 044), enolase, glucokinase, glucose-6-phosphate isomerase, glyceraldehyde-3-phosphate-dehydrogenase (GAP or GAPDH), hexokinase, phosphofructokinase, 3-phosphoglycerate mutase, and pyruvate kinase (PyK) (EPO-A-0 329 203). The yeast PHO5 gene, encoding acid phosphatase, also provides useful promoter sequences [Myanohara et al. (1983) Proc. Natl. Acad. Sci. USA 80:1].

In addition, synthetic promoters which do not occur in nature also function as yeast promoters. For example, UAS sequences of one yeast promoter may be joined with the transcription activation region of another yeast promoter, creating a synthetic hybrid promoter. Examples of such hybrid promoters include the ADH regulatory sequence linked to the GAP transcription activation region (U.S. Pat. Nos. 4,876,197 and 4,880,734). Other examples of hybrid promoters include promoters which consist of the regulatory sequences of either the ADH2, GAL4, GAL10, OR PHO5 genes, combined with the transcriptional activation region of a glycolytic enzyme gene such as GAP or PyK (EP-A-0 164 556). Furthermore, a yeast promoter can include naturally occurring promoters of non-yeast origin that have the ability to bind yeast RNA polymerase and initiate transcription. Examples of such promoters include, inter alia, [Cohen et al. (1980) Proc. Natl. Acad. Sci. USA 77:1078; Henikoff et al. (1981) Nature 283:835; Hollenberg et al. (1981) Curr. Topics Microbiol. Immunol. 96:119; Hollenberg et al. (1979) “The Expression of Bacterial Antibiotic Resistance Genes in the Yeast Saccharomyces cerevisiae,” in: Plasmids of Medical, Environmental and Commercial Importance (eds. K. N. Timmis and A. Puhler); Mercerau-Puigalon et al. (1980) Gene 11:163; Panthier et al. (1980) Curr. Genet. 2:109].

A DNA molecule may be expressed intracellularly in yeast. A promoter sequence may be directly linked with the DNA molecule, in which case the first amino acid at the N-terminus of the recombinant protein will always be a methionine, which is encoded by the ATG start codon. If desired, methionine at the N-terminus may be cleaved from the protein by in vitro incubation with cyanogen bromide.

Fusion proteins provide an alternative for yeast expression systems, as well as in mammalian, baculovirus, and bacterial expression systems. Usually, a DNA sequence encoding the N-terminal portion of an endogenous yeast protein, or other stable protein, is fused to the 5′ end of heterologous coding sequences. Upon expression, this construct will provide a fusion of the two amino acid sequences. For example, the yeast or human superoxide dismutase (SOD) gene, can be linked at the 5′ terminus of a foreign gene and expressed in yeast. The DNA sequence at the junction of the two amino acid sequences may or may not encode a cleavable site. See e.g. EP-A-0 196 056. Another example is a ubiquitin fusion protein. Such a fusion protein is made with the ubiquitin region that preferably retains a site for a processing enzyme (e.g. ubiquitin-specific processing protease) to cleave the ubiquitin from the foreign protein. Through this method, therefore, native foreign protein can be isolated (e.g. WO88/024066).

Alternatively, foreign proteins can also be secreted from the cell into the growth media by creating chimeric DNA molecules that encode a fusion protein comprised of a leader sequence fragment that provide for secretion in yeast of the foreign protein. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell.

DNA encoding suitable signal sequences can be derived from genes for secreted yeast proteins, such as the genes for invertase (EP-A-0012873; JPO 62,096,086) and A-factor (U.S. Pat. No. 4,588,684). Alternatively, leaders of non-yeast origin exit, such as an interferon leader, that also provide for secretion in yeast (EP-A-0060057).

A preferred class of secretion leaders are those that employ a fragment of the yeast alpha-factor gene, which contains both a “pre” signal sequence, and a “pro” region. The types of alpha-factor fragments that can be employed include the full-length pre-pro alpha factor leader (about 83 amino acid residues) as well as truncated alpha-factor leaders (usually about 25 to about 50 amino acid residues) (U.S. Pat. Nos. 4,546,083 and 4,870,008; EP-A-0 324 274). Additional leaders employing an alpha-factor leader fragment that provides for secretion include hybrid alpha-factor leaders made with a presequence of a first yeast, but a pro-region from a second yeast alphafactor. (e.g. see WO 89/02463.)

Usually, transcription termination sequences recognized by yeast are regulatory regions located 3′ to the translation stop codon, and thus together with the promoter flank the coding sequence. These sequences direct the transcription of an mRNA which can be translated into the polypeptide encoded by the DNA. Examples of transcription terminator sequence and other yeast-recognized termination sequences, such as those coding for glycolytic enzymes.

Usually, the above described components, comprising a promoter, leader (if desired), coding sequence of interest, and transcription termination sequence, are put together into expression constructs. Expression constructs are often maintained in a replicon, such as an extrachromosomal element (e.g. plasmids) capable of stable maintenance in a host, such as yeast or bacteria. The replicon may have two replication systems, thus allowing it to be maintained, for example, in yeast for expression and in a prokaryotic host for cloning and amplification. Examples of such yeast-bacteria shuttle vectors include YEp24 [Botstein et al. (1979) Gene 8:17-24], pCl/1 [Brake et al. (1984) Proc. Natl. Acad. Sci USA 81:4642-4646], and YRp17 [Stinchcomb et al. (1982) J. Mol. Biol. 158:157]. In addition, a replicon may be either a high or low copy number plasmid. A high copy number plasmid will generally have a copy number ranging from about 5 to about 200, and usually about 10 to about 150. A host containing a high copy number plasmid will preferably have at least about 10, and more preferably at least about 20. Enter a high or low copy number vector may be selected, depending upon the effect of the vector and the foreign protein on the host. See e.g. Brake et al., supra.

Alternatively, the expression constructs can be integrated into the yeast genome with an integrating vector. Integrating vectors usually contain at least one sequence homologous to a yeast chromosome that allows the vector to integrate, and preferably contain two homologous sequences flanking the expression construct. Integrations appear to result from recombinations between homologous DNA in the vector and the yeast chromosome [Orr-Weaver et al. (1983) Methods in Enzymol. 101:228-245]. An integrating vector may be directed to a specific locus in yeast by selecting the appropriate homologous sequence for inclusion in the vector. See Orr-Weaver et al., supra. One or more expression construct may integrate, possibly affecting levels of recombinant protein produced [Rine et al. (1983) Proc. Natl. Acad. Sci. USA 80:6750]. The chromosomal sequences included in the vector can occur either as a single segment in the vector, which results in the integration of the entire vector, or two segments homologous to adjacent segments in the chromosome and flanking the expression construct in the vector, which can result in the stable integration of only the expression construct.

Usually, extrachromosomal and integrating expression constructs may contain selectable markers to allow for the selection of yeast strains that have been transformed. Selectable markers may include biosynthetic genes that can be expressed in the yeast host, such as ADE2, HIS4, LEU2, TRP1, and ALG7, and the G418 resistance gene, which confer resistance in yeast cells to tunicamycin and G418, respectively. In addition, a suitable selectable marker may also provide yeast with the ability to grow in the presence of toxic compounds, such as metal. For example, the presence of CUP1 allows yeast to grow in the presence of copper ions [Butt et al. (1987) Microbiol, Rev. 51:351].

Alternatively, some of the above described components can be put together into transformation vectors. Transformation vectors are usually comprised of a selectable marker that is either maintained in a replicon or developed into an integrating vector, as described above.

Expression and transformation vectors, either extrachromosomal replicons or integrating vectors, have been developed for transformation into many yeasts. For example, expression vectors have been developed for, inter alia, the following yeasts:Candida albicans [Kurtz, et al. (1986) Mol. Cell. Biol. 6:142], Candida maltosa [Kunze, et al. (1985) J. Basic Microbiol. 25:141]. Hansenula polymorpha [Gleeson, et al. (1986) J. Gen. Microbiol. 132:3459; Roggenkamp et al. (1986) Mol. Gen. Genet. 202:302], Kluyveromyces fragilis [Das, et al. (1984) J. Bacteriol. 158:1165], Kluyveromyces lactis [De Louvencourt et al. (1983) J. Bacteriol. 154:737; Van den Berg et al. (1990) Bio/Technology 8:135], Pichia guillerimondii [Kunze et al. (1985) J. Basic Microbiol. 25:141], Pichia pastoris [Cregg, et al. (1985) Mol. Cell. Biol. 5:3376; U.S. Pat. Nos. 4,837,148 and 4,929,555], Saccharomyces cerevisiae [Hinnen et al. (1978) Proc. Natl. Acad. Sci. USA 75:1929; Ito et al. (1983) J. Bacteriol. 153:163], Schizosaccharomyces pombe [Beach and Nurse (1981) Nature 300:706], and Yarrowia lipolytica [Davidow, et al. (1985) Curr. Genet. 10:380471 Gaillardin, et al. (1985) Curr. Genet. 10:49].

Methods of introducing exogenous DNA into yeast hosts are well-known in the art, and usually include either the transformation of spheroplasts or of intact yeast cells treated with alkali cations. Transformation procedures usually vary with the yeast species to be transformed. See e.g. [Kurtz et al. (1986) Mol. Cell. Biol. 6:142; Kunze et al. (1985) J. Basic Microbiol. 25:141; Candida]; [Gleeson et al. (1986) J. Gen. Microbiol. 132:3459; Roggenkamp et al. (1986) Mol. Gen. Genet. 202:302; Hansenula]; [Das et al. (1984) J. Bacteriol. 158:1165; De Louvencourt et al. (1983) J. Bacteriol. 154:1165; Van den Berg et al. (1990) Bio/Technology 8:135; Kluyveromyces]; [Cregg et al. (1985) Mol. Cell. Biol. 5:3376; Kunze et al. (1985) J. Basic Microbiol. 25:141; U.S. Pat. Nos. 4,837,148 & 4,929,555; Pichia]; [Hinnen et al. (1978) Proc. Natl. Acad. Sci. USA 75;1929; Ito et al. (1983) J. Bacteriol. 153:163 Saccharomyces]; [Beach & Nurse (1981) Nature 300:706; Schizosaccharomyces]; [Davidow et al. (1985) Curr. Genet. 10:39; Gaillardin et al. (1985) Curr. Genet. 10:49; Yarrowia].

Pharmaceutical Compositions

Pharmaceutical compositions can comprise polypeptides and/or nucleic acid of the invention. The pharmaceutical compositions will comprise a therapeutically effective amount of either polypeptides, antibodies, or polynucleotides of the claimed invention.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgement of the clinician.

For purposes of the present invention, an effective dose will be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

Delivery Methods

Once formulated, the compositions of the invention can be administered directly to the subject. The subjects to be treated can be animals; in particular, human subjects can be treated.

Direct delivery of the compositions will generally be accomplished by injection, either subcutaneously, intraperitoneally, intravenously or intramuscularly or delivered to the interstitial space of a tissue. The compositions can also be administered into a lesion. Other modes of administration include oral and pulmonary administration, suppositories, and transdermal or transcutaneous applications (e.g. see WO98/20734), needles, and gene guns or hyposprays. Dosage treatment may be a single dose schedule or a multiple dose schedule.

Vaccines

Vaccines according to the invention may either be prophylactic (ie. to prevent infection) or therapeutic (ie. to treat disease after infection).

Such vaccines comprise immunizing antigen(s), immunogen(s), polypeptide(s), protein(s) or nucleic acid, usually in combination with “pharmaceutically acceptable carriers,” which include any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, etc. pathogens.

Preferred adjuvants to enhance effectiveness of the composition include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) MF59™ (WO 90/14837; Chapter 10 in Vaccine design: the subunit and adjuvant approach, eds. Powell & Newman, Plenum Press 1995), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, Mass.), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (3) saponin adjuvants, such as Stimulon™ (Cambridge Bioscience, Worcester, Mass.) may be used or particles generated therefrom such as ISCOMs (immunostimulating complexes); (4) Complete Freund's Adjuvant (CFA) and Incomplete Freund's Adjuvant (IFA); (5) cytokines, such as interleukins (e.g. IL-1, IL-2, IL-4, IL-5, IL-6, IL-7, IL-12, etc.), interferons (e.g. gamma interferon), macrophage colony stimulating factor (M-CSF), tumor necrosis factor (TNF), etc; and (6) other substances that act as immunostimulating agents to enhance the effectiveness of the composition. Alum and MF59™ are preferred.

As mentioned above, muramyl peptides include, but are not limited to, N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (MTP-PE), etc.

The immunogenic compositions (e.g. the immunizing antigen/immunogen/polypeptide/protein/nucleic acid, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic compositions used as vaccines comprise an immunologically effective amount of the antigenic or immunogenic polypeptides, as well as any other of the above-mentioned components, as needed. By “immunologically effective amount”, it is meant that the administration of that amount to an individual, either in a single dose or as part of a series, is effective for treatment or prevention. This amount varies depending upon the health and physical condition of the individual to be treated, the taxonomic group of individual to be treated (e.g. nonhuman primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

The immunogenic compositions are conventionally administered parenterally, e.g. by injection, either subcutaneously, intramuscularly, or transdermally/transcutaneously (e.g. WO98/20734). Additional formulations suitable for other modes of administration include oral and pulmonary formulations, suppositories, and transdermal applications. Dosage treatment may be a single dose schedule or a multiple dose schedule. The vaccine may be administered in conjunction with other immunoregulatory agents.

As an alternative to protein-based vaccines, DNA vaccination may be employed [e.g. Robinson & Torres (1997) Seminars in Immunology 9:271-283; Donnelly et al. (1997) Annu Rev Immunol 15:617-648; see later herein].

Gene Delivery Vehicles

Gene therapy vehicles for delivery of constructs including a coding sequence of a therapeutic of the invention, to be delivered to the mammal for expression in the mammal, can be administered either locally or systemically. These constructs can utilize viral or non-viral vector approaches in in vivo or ex vivo modality. Expression of such coding sequence can be induced using endogenous mammalian or heterologous promoters. Expression of the coding sequence in vivo can be either constitutive or regulated.

The invention includes gene delivery vehicles capable of expressing the contemplated nucleic acid sequences. The gene delivery vehicle is preferably a viral vector and, more preferably, a retroviral, adenoviral, adeno-associated viral (AAV), herpes viral, or alphavirus vector. The viral vector can also be an astrovirus, coronavirus, orthomyxovirus, papovavirus, paramyxovirus, parvovirus, picornavirus, poxvirus, or togavirus viral vector. See generally, Jolly (1994) Cancer Gene Therapy 1:51-64; Kimura (1994) Human Gene Therapy 5:845-852; Connelly (1995) Human Gene Therapy 6:185-193; and Kaplitt (1994) Nature Genetics 6:148-153.

Retroviral vectors are well known in the art and we contemplate that any retroviral gene therapy vector is employable in the invention, including B, C and D type retroviruses, xenotropic retroviruses (for example, NZB-X1, NZB-X2 and NZB9-1 (see O'Neill (1985) J. Virol. 53:160) polytropic retroviruses e.g. MCF and MCF-MLV (see Kelly (1983) J. Virol. 45:291), spumaviruses and lentiviruses. See RNA Tumor Viruses, Second Edition, Cold Spring Harbor Laboratory, 1985.

Portions of the retroviral gene therapy vector may be derived from different retroviruses. For example, retrovector LTRs may be derived from a Murine Sarcoma Virus, a tRNA binding site from a Rous Sarcoma Virus, a packaging signal from a Murine Leukemia Virus, and an origin of second strand synthesis from an Avian Leukosis Virus.

These recombinant retroviral vectors may be used to generate transduction competent retroviral vector particles by introducing them into appropriate packaging cell lines (see U.S. Pat. No. 5,591,624). Retrovirus vectors can be constructed for site-specific integration into host cell DNA by incorporation of a chimeric integrase enzyme into the retroviral particle (see WO96/37626). It is preferable that the recombinant viral vector is a replication defective recombinant virus.

Packaging cell lines suitable for use with the above-described retrovirus vectors are well known in the art, are readily prepared (see WO95/30763 and WO92/05266), and can be used to create producer cell lines (also termed vector cell lines or “VCLs”) for the production of recombinant vector particles. Preferably, the packaging cell lines are made from human parent cells (e.g. HT1080 cells) or mink parent cell lines, which eliminates inactivation in human serum.

Preferred retroviruses for the construction of retroviral gene therapy vectors include Avian Leukosis Virus, Bovine Leukemia, Virus, Murine Leukemia Virus, Mink-Cell Focus-Inducing Virus, Murine Sarcoma Virus, Reticuloendotheliosis Virus and Rous Sarcoma Virus. Particularly preferred Murine Leukemia Viruses include 4070A and 1504A (Hartley and Rowe (1976) J Virol 19:19-25), Abelson (ATCC No. VR-999), Friend (ATCC No. VR-245), Graffi, Gross (ATCC No1 VR-590), Kirsten, Harvey Sarcoma Virus and Rauscher (ATCC No. VR-998) and Moloney Murine Leukemia Virus (ATCC No. VR-190). Such retroviruses may be obtained from depositories or collections such as the American Type Culture Collection (“ATCC”) in Rockville, Md. or isolated from known sources using commonly available techniques.

Exemplary known retroviral gene therapy vectors employable in this invention include those described in patent applications GB2200651, EP0415731, EP0345242, EP0334301, WO89/02468; WO89/05349, WO89/09271, WO90/02806, WO90/07936, WO94/03622, WO93/25698, WO93/25234, WO93/11230, WO93/10218, WO91/02805, WO91/02825, WO95/07994, U.S. Pat. No. 5,219,740, U.S. Pat. No. 4,405,712, U.S. Pat. No. 4,861,719, U.S. Pat. No. 4,980,289, U.S. Pat. No. 4,777,127, U.S. Pat. No. 5,591,624. See also Vile (1993) Cancer Res 53:3860-3864; Vile (1993) Cancer Res 53:962-867; Ram (1993) Cancer Res 53 (1993) 83-88; Takamiya (1992) J Neurosci Res 33:493-503; Baba (1993) J Neurosurg 79:729-735; Mann (1983) Cell 33:153; Cane (1984) Proc Natl Acad Sci 81:6349; and Miller (1990) Human Gene Therapy 1.

Human adenoviral gene therapy vectors are also known in the art and employable in this invention. See, for example, Berkner (1988) Biotechniques 6:616 and Rosenfeld (1991) Science 252:431, and WO93/07283, WO93/06223, and WO93/07282. Exemplary known adenoviral gene therapy vectors employable in this invention include those described in the above referenced documents and in WO94/12649, WO93/03769, WO93/19191, WO94/28938, WO95/11984, WO95/00655, WO95/27071, WO95/29993, WO95/34671, WO96/05320, WO94/08026, WO94/11506, WO93/06223, WO94/24299, WO95/14102, WO95/24297, WO95/02697, WO94/28152, WO94/24299, WO95/09241, WO95/25807, WO95/05835, WO94/18922 and WO95/09654. Alternatively, administration of DNA linked to killed adenovirus as described in Curiel (1992) Hum. Gene Ther. 3:147-154 may be employed. The gene delivery vehicles of the invention also include adenovirus associated virus (AAV) vectors. Leading and preferred examples of such vectors for use in this invention are the AAV-2 based vectors disclosed in Srivastava, WO93/09239. Most preferred AAV vectors comprise the two AAV inverted terminal repeats in which the native D-sequences are modified by substitution of nucleotides, such that at least 5 native nucleotides and up to 18 native nucleotides, preferably at least 10 native nucleotides up to 18 native nucleotides, most preferably 10 native nucleotides are retained and the remaining nucleotides of the D-sequence are deleted or replaced with non-native nucleotides. The native D-sequences of the AAV inverted terminal repeats are sequences of 20 consecutive nucleotides in each AAV inverted terminal repeat (ie. there is one sequence at each end) which are not involved in HP formation. The non-native replacement nucleotide may be any nucleotide other than the nucleotide found in the native D-sequence in the same position. Other employable exemplary AAV vectors are pWP-19, pWN-1, both of which are disclosed in Nahreini (1993) Gene 124:257-262. Another example of such an AAV vector is psub201 (see Samulski (1987) J. Virol. 61:3096). Another exemplary AAV vector is the Double-D ITR vector. Construction of the Double-D ITR vector is disclosed in U.S. Pat. No. 5,478,745. Still other vectors are those disclosed in Carter U.S. Pat. No. 4,797,368 and Muzyczka U.S. Pat. No. 5,139,941, Chartejee U.S. Pat. No. 5,474,935, and Kotin WO94/288157. Yet a further example of an AAV vector employable in this invention is SSV9AFABTKneo, which contains the AFP enhancer and albumin promoter and directs expression predominantly in the liver. Its structure and construction are disclosed in Su (1996) Human Gene Therapy 7:463-470. Additional AAV gene therapy vectors are described in U.S. Pat. No. 5,354,678, U.S. Pat. No. 5,173,414, U.S. Pat. No. 5,139,941, and U.S. Pat. No. 5,252,479.

The gene therapy vectors of the invention also include herpes vectors. Leading and preferred examples are herpes simplex virus vectors containing a sequence encoding a thymidine kinase polypeptide such as those disclosed in U.S. Pat. No. 5,288,641 and EP0176170 (Roizman). Additional exemplary herpes simplex virus vectors include HFEM/ICP6-LacZ disclosed in WO95/04139 (Wistar), pHSVlac described in Geller (1988) Science 241:1667-1669 and in WO90/09441 & WO92/07945, HSV Us3::pgC-lacZ described in Fink (1992) Human Gene Therapy 3:11-19 and HSV 7134, 2 RH 105 and GAL4 described in EP 0453242 (Breakefield), and those deposited with ATCC as accession numbers ATCC VR-977 and ATCC VR-260.

Also contemplated are alpha virus gene therapy vectors that can be employed in this invention. Preferred alpha virus vectors are Sindbis viruses vectors. Togaviruses, Semliki Forest virus (ATCC VR-67; ATCC VR-1247), Middleberg virus (ATCC VR-370), Ross River virus (ATCC VR-373; ATCC VR-1246), Venezuelan equine encephalitis virus (ATCC VR923; ATCC VR-1250; ATCC VR-1249; ATCC VR-532), and those described in U.S. Pat. Nos. 5,091,309, 5,217,879, and WO92/10578. More particularly, those alpha virus vectors described in U.S. Ser. No. 08/405,627, filed Mar. 15, 1995, WO94/21792, WO92/10578, WO95/07994, U.S. Pat. No. 5,091,309 and U.S. Pat. No. 5,217,879 are employable. Such alpha viruses may be obtained from depositories or collections such as the ATCC in Rockville, Md. or isolated from known sources using commonly available techniques. Preferably, alphavirus vectors with reduced cytotoxicity are used (see U.S. Ser. No. 08/679,640).

DNA vector systems such as eukaryotic layered expression systems are also useful for expressing the nucleic acids of the invention. See WO95/07994 for a detailed description of eukaryotic layered expression systems. Preferably, the eukaryotic layered expression systems of the invention are derived from alphavirus vectors and most preferably from Sindbis viral vectors.

Other viral vectors suitable for use in the present invention include those derived from poliovirus, for example ATCC VR-58 and those described in Evans, Nature 339 (1989) 385 and Sabin (1973) J. Biol. Standardization 1:115; rhinovirus, for example ATCC VR-1110 and those described in Arnold (1990) J Cell Biochem L401; pox viruses such as canary pox virus or vaccinia virus, for example ATCC VR-111 and ATCC VR-2010 and those described in Fisher-Hoch (1989) Proc Natl Acad Sci 86:317; Flexner (1989) Ann NY Acad Sci 569:86, Flexner (1990) Vaccine 8:17; in U.S. Pat. No. 4,603,112 and U.S. Pat. No. 4,769,330 and WO89/01973; SV40 virus, for example ATCC VR-305 and those described in Mulligan (1979) Nature 277:108 and Madzak (1992) J Gen Virol 73:1533; influenza virus, for example ATCC VR-797 and recombinant influenza viruses made employing reverse genetics techniques as described in U.S. Pat. No. 5,166,057 and in Enami (1990) Proc Natl Acad Sci 87:3802-3805; Enami & Palese (1991) J Virol 65:2711-2713 and Luytjes (1989) Cell 59:110, (see also McMichael (1983) NEJ Med 309:13, and Yap (1978) Nature 273:238 and Nature (1979) 277:108); human immunodeficiency virus as described in EP-0386882 and in Buchschacher (1992) J. Virol. 66:2731; measles virus, for example ATCC VR-67 and VR-1247 and those described in EP-0440219; Aura virus, for example ATCC VR-368; Bebaru virus, for example ATCC VR-600 and ATCC VR-1240; Cabassou virus, for example ATCC VR-922; Chikungunya virus, for example ATCC VR-64 and ATCC VR-1241; Fort Morgan Virus, for example ATCC VR-924; Getah virus, for example ATCC VR-369 and ATCC VR-1243; Kyzylagach virus, for example ATCC VR-927; Mayaro virus, for example ATCC VR-66; Mucambo virus, for example ATCC VR-580 and ATCC VR-1244; Ndumu virus, for example ATCC VR-371; Pixuna virus, for example ATCC VR-372 and ATCC VR-1245; Tonate virus, for example ATCC VR-925; Triniti virus, for example ATCC VR-469; Una virus, for example ATCC VR-374; Whataroa virus, for example ATCC VR-926; Y-62-33 virus, for example ATCC VR-375; O'Nyong virus, Eastern encephalitis virus, for example ATCC VR-65 and ATCC VR-1242; Western encephalitis virus, for example ATCC VR-70, ATCC VR-1251, ATCC VR-622 and ATCC VR-1252; and coronavirus, for example ATCC VR-740 and those described in Hamre (1966) Proc Soc Exp Biol Med 121:190.

Delivery of the compositions of this invention into cells is not limited to the above mentioned viral vectors. Other delivery methods and media may be employed such as, for example, nucleic acid expression vectors, polycationic condensed DNA linked or unlinked to killed adenovirus alone, for example see U.S. Ser. No. 08/366,787, filed Dec. 30, 1994 and Curiel (1992) Hum Gene Ther 3:147-154 ligand linked DNA, for example see Wu (1989) J Biol Chem 264:16985-16987, eucaryotic cell delivery vehicles cells, for example see U.S. Ser. No. 08/240,030, filed May 9, 1994, and U.S. Ser. No. 08/404,796, deposition of photopolymerized hydrogel materials, hand-held gene transfer particle gun, as described in U.S. Pat. No. 5,149,655, ionizing radiation as described in U.S. Pat. No. 5,206,152 and in WO92/11033, nucleic charge neutralization or fusion with cell membranes. Additional approaches are described in Philip (1994) Mol Cell Biol 14:2411-2418 and in Woffendin (1994) Proc Natl Acad Sci 91:1581-1585.

Particle mediated gene transfer may be employed, for example see U.S. Ser. No. 60/023,867. Briefly, the sequence can be inserted into conventional vectors that contain conventional control sequences for high level expression, and then incubated with synthetic gene transfer molecules such as polymeric DNA-binding cations like polylysine, protamine, and albumin, linked to cell targeting ligands such as asialoorosomucoid, as described in Wu & Wu (1987) J. Biol. Chem. 262:4429-4432, insulin as described in Hucked (1990) Biochem Pharmacol 40:253-263, galactose as described in Plank (1992) Bioconjugate Chem 3:533-539, lactose or transferrin.

Naked DNA may also be employed. Exemplary naked DNA introduction methods are described in WO90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm.

Liposomes that can act as gene delivery vehicles are described in U.S. Pat. No. 5,422,120, WO95/13796, WO94/23697, WO91/14445 and EP-524,968. As described in U.S. Ser. No. 60/023,867, on non-viral delivery, the nucleic acid sequences encoding a polypeptide can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, insulin, galactose, lactose, or transferrin. Other delivery systems include the use of liposomes to encapsulate DNA comprising the gene under the control of a variety of tissue-specific or ubiquitously-active promoters. Further non-viral delivery suitable for use includes mechanical delivery systems such as the approach described in Woffendin et al (1994) Proc. Natl. Acad. Sci. USA 91(24):11581-11585. Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and WO92/11033

Exemplary liposome and polycationic gene delivery vehicles are those described in U.S. Pat. Nos. 5,422,120 and 4,762,915; in WO 95/13796; WO94/23697; and WO91/14445; in EP-0524968; and in Stryer, Biochemistry, pages 236-240 (1975) W. H. Freeman, San Francisco; Szoka (1980) Biochem Biophys Acta 600:1; Bayer (1979) Biochem Biophys Acta 550:464; Rivnay (1987) Meth Enzymol 149:119; Wang (1987) Proc Natl Acad Sci 84:7851; Plant (1989) Anal Biochem 176:420.

A polynucleotide composition can comprises therapeutically effective amount of a gene therapy vehicle, as the term is defined above. For purposes of the present invention, an effective dose will be from about 0.01 mg/kg to 50 mg/kg or 0.05 mg/kg to about 10 mg/kg of the DNA constructs in the individual to which it is administered.

Delivery Methods

Once formulated, the polynucleotide compositions of the invention can be administered (1) directly to the subject; (2) delivered ex vivo, to cells derived from the subject; or (3) in vitro for recombinant protein expression. The subjects to be treated can be mammals or birds. Also, human subjects can be treated.

Direct delivery of the compositions will generally be accomplished by injection, either subcutaneously, intraperitoneally, intravenously or intramuscularly or delivered to the interstitial space of a tissue. The compositions can also be administered into a lesion. Other modes of administration include oral and pulmonary administration, suppositories, and transdermal or transcutaneous applications (e.g. see WO98/20734), needles, and gene guns or hyposprays. Dosage treatment may be a single dose schedule or a multiple dose schedule.

Methods for the ex vivo delivery and reimplantation of transformed cells into a subject are known in the art and described in e.g. WO93/14778. Examples of cells useful in ex vivo applications include, for example, stem cells, particularly hematopoetic, lymph cells, macrophages, dendritic cells, or tumor cells.

Generally, delivery of nucleic acids for both ex vivo and in vitro applications can be accomplished by the following procedures, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Polynucleotide and Polypeptide Pharmaceutical Compositions

In addition to the pharmaceutically acceptable carriers and salts described above, the following additional agents can be used with polynucleotide and/or polypeptide compositions.

A. Polypeptides

One example are polypeptides which include, without limitation: asioloorosomucoid (ASOR); transferrin; asialoglycoproteins; antibodies; antibody fragments; ferritin; interleukins; interferons, granulocyte, macrophage colony stimulating factor (GM-CSF), granulocyte colony stimulating factor (G-CSF), macrophage colony stimulating factor (M-CSF), stem cell factor and erythropoietin. Viral antigens, such as envelope proteins, can also be used. Also, proteins from other invasive organisms, such as the 17 amino acid peptide from the circumsporozoite protein of plasmodium falciparum known as RII.

B. Hormones, Vitamins, etc.

Other groups that can be included are, for example: hormones, steroids, androgens, estrogens, thyroid hormone, or vitamins, folic acid.

C. Polyalkylenes, Polysaccharides, etc.

Also, polyalkylene glycol can be included with the desired polynucleotides/polypeptides. In a preferred embodiment, the polyalkylene glycol is polyethlylene glycol. In addition, mono-, di-, or polysaccharides can be included. In a preferred embodiment of this aspect, the polysaccharide is dextran or DEAE-dextran. Also, chitosan and poly(lactide-co-glycolide)

D. Lipids, and Liposomes

The desired polynucleotide/polypeptide can also be encapsulated in lipids or packaged in liposomes prior to delivery to the subject or to cells derived therefrom.

Lipid encapsulation is generally accomplished using liposomes which are able to stably bind or entrap and retain nucleic acid. The ratio of condensed polynucleotide to lipid preparation can vary but will generally be around 1:1 (mg DNA:micromoles lipid), or more of lipid. For a review of the use of liposomes as carriers for delivery of nucleic acids, see, Hug and Sleight (1991) Biochim. Biophys. Acta. 1097:1-17; Straubinger (1983) Meth. Enzymol. 101:512-527.

Liposomal preparations for use in the present invention include cationic (positively charged), anionic (negatively charged) and neutral preparations. Cationic liposomes have been shown to mediate intracellular delivery of plasmid DNA (Felgner (1987) Proc. Natl. Acad. Sci. USA 84:7413-7416); mRNA (Malone (1989) Proc. Natl. Acad. Sci. USA 86:6077-6081); and purified transcription factors (Debs (1990) J. Biol. Chem. 265:10189-10192), in functional form.

Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. (See, also, Felgner supra). Other commercially available liposomes include transfectace (DDAB/DOPE) and DOTAP/DOPE (Boerhinger). Other cationic liposomes can be prepared from readily available materials using techniques well known in the art. See, e.g. Szoka (1978) Proc. Natl. Acad. Sci. USA 75:4194-4198; WO90/11092 for a description of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See e.g. Straubinger (1983) Meth. Immunol. 101:512-527; Szoka (1978) Proc. Natl. Acad. Sci. USA 75:4194-4198; Papahadjopoulos (1975) Biochim. Biophys. Acta 394:483; Wilson (1979) Cell 17:77); Deamer & Bangham (1976) Biochim. Biophys. Acta 443:629; Ostro (1977) Biochem. Biophys. Res. Commun. 76:836; Fraley (1979) Proc. Natl. Acad. Sci. USA 76:3348); Enoch & Strittmatter (1979) Proc. Natl. Acad. Sci. USA 76:145; Fraley (1980) J. Biol. Chem. (1980) 255:10431; Szoka & Papahadjopoulos (1978) Proc. Natl. Acad. Sci. USA 75:145; and Schaefer-Ridder (1982) Science 215:166.

E. Lipoproteins

In addition, lipoproteins can be included with the polynucleotide/polypeptide to be delivered. Examples of lipoproteins to be utilized include: chylomicrons, HDL, IDL, LDL, and VLDL. Mutants, fragments, or fusions of these proteins can also be used. Also, modifications of naturally occurring lipoproteins can be used, such as acetylated LDL. These lipoproteins can target the delivery of polynucleotides to cells expressing lipoprotein receptors. Preferably, if lipoproteins are including with the polynucleotide to be delivered, no other targeting ligand is included in the composition.

Naturally occurring lipoproteins comprise a lipid and a protein portion. The protein portion are known as apoproteins. At the present, apoproteins A, B, C, D, and E have been isolated and identified. At least two of these contain several proteins, designated by Roman numerals, AI, AII, AIV; CI, CII, CIII.

A lipoprotein can comprise more than one apoprotein. For example, naturally occurring chylomicrons comprises of A, B, C, & E, over time these lipoproteins lose A and acquire C and E apoproteins. VLDL comprises A, B, C, & E apoproteins, LDL comprises apoprotein B; HDL comprises apoproteins A, C, & E.

The amino acid of these apoproteins are known and are described in, for example, Breslow (1985) Annu Rev. Biochem 54:699; Law (1986) Adv. Exp Med. Biol. 151:162; Chen (1986) J Biol Chem 261:12918; Kane (1980) Proc Natl Acad Sci USA 77:2465; and Utermann (1984) Hum Genet 65:232.

Lipoproteins contain a variety of lipids including, triglycerides, cholesterol (free and esters), and phospholipids. The composition of the lipids varies in naturally occurring lipoproteins. For example, chylomicrons comprise mainly triglycerides. A more detailed description of the lipid content of naturally occurring lipoproteins can be found, for example, in Meth. Enzymol. 128 (1986). The composition of the lipids are chosen to aid in conformation of the apoprotein for receptor binding activity. The composition of lipids can also be chosen to facilitate hydrophobic interaction and association with the polynucleotide binding molecule.

Naturally occurring lipoproteins can be isolated from serum by ultracentrifugation, for instance. Such methods are described in Meth. Enzymol. (supra); Pitas (1980) J. Biochem. 255:5454-5460 and Mahey (1979) J Clin. Invest 64:743-750. Lipoproteins can also be produced by in vitro or recombinant methods by expression of the apoprotein genes in a desired host cell. See, for example, Atkinson (1986) Annu Rev Biophys Chem 15:403 and Radding (1958) Biochim Biophys Acta 30: 443. Lipoproteins can also be purchased from commercial suppliers, such as Biomedical Techniologies, Inc., Stoughton, Mass., USA. Further description of lipoproteins can be found in Zuckermann et al. PCT/US97/14465.

F. Polycationic Agents

Polycationic agents can be included, with or without lipoprotein, in a composition with the desired polynucleotide/polypeptide to be delivered.

Polycationic agents, typically, exhibit a net positive charge at physiological relevant pH and are capable of neutralizing the electrical charge of nucleic acids to facilitate delivery to a desired location. These agents have both in vitro, ex vivo, and in vivo applications. Polycationic agents can be used to deliver nucleic acids to a living subject either intramuscularly, subcutaneously, etc.

The following are examples of useful polypeptides as polycationic agents: polylysine, polyarginine, polyornithine, and protamine. Other examples include histones, protamines, human serum albumin, DNA binding proteins, non-histone chromosomal proteins, coat proteins from DNA viruses, such as (X174, transcriptional factors also contain domains that bind DNA and therefore may be useful as nucleic aid condensing agents. Briefly, transcriptional factors such as C/CEBP, c-jun, c-fos, AP-1, AP-2, AP-3, CPF, Prot-1, Sp-1, Oct-1, Oct-2, CREP, and TFIID contain basic domains that bind DNA sequences.

Organic polycationic agents include: spermine, spermidine, and purtrescine.

The dimensions and of the physical properties of a polycationic agent can be extrapolated from the list above, to construct other polypeptide polycationic agents or to produce synthetic polycationic agents.

Synthetic polycationic agents which are useful include, for example, DEAE-dextran, polybrene. Lipofectin™, and lipofectAMINE™ are monomers that form polycationic complexes when combined with polynucleotides/polypeptides.

Nucleic Acid Hybridisation

“Hybridization” refers to the association of two nucleic acid sequences to one another by hydrogen bonding. Typically, one sequence will be fixed to a solid support and the other will be free in solution. Then, the two sequences will be placed in contact with one another under conditions that favor hydrogen bonding. Factors that affect this bonding include: the type and volume of solvent; reaction temperature; time of hybridization; agitation; agents to block the non-specific attachment of the liquid phase sequence to the solid support (Denhardt's reagent or BLOTTO); concentration of the sequences; use of compounds to increase the rate of association of sequences (dextran sulfate or polyethylene glycol); and the stringency of the washing conditions following hybridization. See Sambrook et al. [supra] vol. 2, chapt. 9, pp. 9.47 to 9.57.

“Stringency” refers to conditions in a hybridization reaction that favor association of very similar sequences over sequences that differ. For example, the combination of temperature and salt concentration should be chosen that is approximately 120 to 200° C. below the calculated Tm of the hybrid under study. The temperature and salt conditions can often be determined empirically in preliminary experiments in which samples of genomic DNA immobilized on filters are hybridized to the sequence of interest and then washed under conditions of different stringencies. See Sambrook et al. at page 9.50.

Variables to consider when performing, for example, a Southern blot are (1) the complexity of the DNA being blotted and (2) the homology between the probe and the sequences being detected. The total amount of the fragment(s) to be studied can vary a magnitude of 10, from 0.1 to 1 μg for a plasmid or phage digest to 10⁻⁹ to 10⁻⁸ g for a single copy gene in a highly complex eukaryotic genome. For lower complexity polynucleotides, substantially shorter blotting, hybridization, and exposure times, a smaller amount of starting polynucleotides, and lower specific activity of probes can be used. For example, a single-copy yeast gene can be detected with an exposure time of only 1 hour starting with 1 μg of yeast DNA, blotting for two hours, and hybridizing for 4-8 hours with a probe of 10⁸ cpm/μg. For a single-copy mammalian gene a conservative approach would start with 10 μg of DNA, blot overnight, and hybridize overnight in the presence of 10% dextran sulfate using a probe of greater than 10⁸ cpm/μg, resulting in an exposure time of ˜24 hours.

Several factors can affect the melting temperature (Tm) of a DNA-DNA hybrid between the probe and the fragment of interest, and consequently, the appropriate conditions for hybridization and washing. In many cases the probe is not 100% homologous to the fragment. Other commonly encountered variables include the length and total G+C content of the hybridizing sequences and the ionic strength and formamide content of the hybridization buffer. The effects of all of these factors can be approximated by a single equation:

Tm=81+16.6(log₁₀Ci)+0.4[%(G+C)]−0.6(% formamide)−600/n−1.5(% mismatch).

where Ci is the salt concentration (monovalent ions) and n is the length of the hybrid in base pairs (slightly modified from Meinkoth & Wahl (1984) Anal. Biochem. 138: 267-284).

In designing a hybridization experiment, some factors affecting nucleic acid hybridization can be conveniently altered. The temperature of the hybridization and washes and the salt concentration during the washes are the simplest to adjust. As the temperature of the hybridization increases (ie. stringency), it becomes less likely for hybridization to occur between strands that are nonhomologous, and as a result, background decreases. If the radiolabeled probe is not completely homologous with the immobilized fragment (as is frequently the case in gene family and interspecies hybridization experiments), the hybridization temperature must be reduced, and background will increase. The temperature of the washes affects the intensity of the hybridizing band and the degree of background in a similar manner. The stringency of the washes is also increased with decreasing salt concentrations.

In general, convenient hybridization temperatures in the presence of 50% formamide are 42° C. for a probe with is 95% to 100% homologous to the target fragment, 37° C. for 90% to 95% homology, and 32° C. for 85% to 90% homology. For lower homologies, formamide content should be lowered and temperature adjusted accordingly, using the equation above. If the homology between the probe and the target fragment are not known, the simplest approach is to start with both hybridization and wash conditions which are nonstringent. If non-specific bands or high background are observed after autoradiography, the filter can be washed at high stringency and reexposed. If the time required for exposure makes this approach impractical, several hybridization and/or washing stringencies should be tested in parallel.

Nucleic Acid Probe Assays

Methods such as PCR, branched DNA probe assays, or blotting techniques utilizing nucleic acid probes according to the invention can determine the presence of cDNA or mRNA. A probe is said to “hybridize” with a sequence of the invention if it can form a duplex or double stranded complex, which is stable enough to be detected.

The nucleic acid probes will hybridize to the Chlamydial nucleotide sequences of the invention (including both sense and antisense strands). Though many different nucleotide sequences will encode the amino acid sequence, the native Chlamydial sequence is preferred because it is the actual sequence present in cells. mRNA represents a coding sequence and so a probe should be complementary to the coding sequence; single-stranded cDNA is complementary to mRNA, and so a cDNA probe should be complementary to the non-coding sequence.

The probe sequence need not be identical to the Chlamydial sequence (or its complement)—some variation in the sequence and length can lead to increased assay sensitivity if the nucleic acid probe can form a duplex with target nucleotides, which can be detected. Also, the nucleic acid probe can include additional nucleotides to stabilize the formed duplex. Additional Chlamydial sequence may also be helpful as a label to detect the formed duplex. For example, a non-complementary nucleotide sequence may be attached to the 5′ end of the probe, with the remainder of the probe sequence being complementary to a Chlamydial sequence. Alternatively, non-complementary bases or longer sequences can be interspersed into the probe, provided that the probe sequence has sufficient complementarity with the a Chlamydial sequence in order to hybridize therewith and thereby form a duplex which can be detected.

The exact length and sequence of the probe will depend on the hybridization conditions, such as temperature, salt condition and the like. For example, for diagnostic applications, depending on the complexity of the analyte sequence, the nucleic acid probe typically contains at least 10-20 nucleotides, preferably 15-25, and more preferably ≧30 nucleotides, although it may be shorter than this. Short primers generally require cooler temperatures to form sufficiently stable hybrid complexes with the template.

Probes may be produced by synthetic procedures, such as the triester method of Matteucci et al. [J. Am. Chem. Soc. (1981) 103:3185], or according to Urdea et al. [Proc. Natl. Acad. Sci. USA (1983) 80: 7461], or using commercially available automated oligonucleotide synthesizers.

The chemical nature of the probe can be selected according to preference. For certain applications, DNA or RNA are appropriate. For other applications, modifications may be incorporated e.g. backbone modifications, such as phosphorothioates or methylphosphonates, can be used to increase in vivo half-life, alter RNA affinity, increase nuclease resistance etc. [e.g. see Agrawal & Iyer (1995) Curr Opin Biotechnol 6:12-19; Agrawal (1996) TIBTECH 14:376-387]; analogues such as peptide nucleic acids may also be used [e.g. see Corey (1997) TIBTECH 15:224-229; Buchardt et al. (1993) TIBTECH 11:384-386].

Alternatively, the polymerase chain reaction (PCR) is another well-known means for detecting small amounts of target nucleic acids. The assay is described in: Mullis et al. [Meth. Enzymol. (1987) 155: 335-350]; U.S. Pat. Nos. 4,683,195 & 4,683,202. Two ‘primers’ hybridize with the target nucleic acids and are used to prime the reaction. The primers can comprise sequence that does not hybridize to the sequence of the amplification target (or its complement) to aid with duplex stability or, for example, to incorporate a convenient restriction site. Typically, such sequence will flank the desired Chlamydial sequence.

A thermostable polymerase creates copies of target nucleic acids from the primers using the original target nucleic acids as a template. After a threshold amount of target nucleic acids are generated by the polymerase, they can be detected by more traditional methods, such as Southern blots. When using the Southern blot method, the labelled probe will hybridize to the Chlamydial sequence (or its complement).

Also, mRNA or cDNA can be detected by traditional blotting techniques described in Sambrook et al [supra]. mRNA, or cDNA generated from mRNA using a polymerase enzyme, can be purified and separated using gel electrophoresis. The nucleic acids on the gel are then blotted onto a solid support, such as nitrocellulose. The solid support is exposed to a labelled probe and then washed to remove any unhybridized probe. Next, the duplexes containing the labeled probe are detected. Typically, the probe is labelled with a radioactive moiety.

### BRIEF DESCRIPTION OF THE DRAWINGS

FIGS. 1A-1C, 2A-2C, 3A-3C, 4A-4C, 5A-5C, 6A-6C, 7A-7C, 8A-8C, 9A-9C, 10A-10B, 11A-11C, 12A-12C, 13A-13B, 14A-14B, 15A-15C, 16A-16C, 17A-17C, 18A-18C, 19A-19B, 20A-20B, 21A-21C, 22A-22C, 23A-23C, 24A-24C, 25A-25C, 26A-26B, 27A-27C, 28A-28C, 29A-29C, 30A-30C, 31A-31B, 32A-32C, 33A-33B, 34A-34C, 35A-35C, 36A-36B, 37A-37D, 38A-38B, 39A-39D, 40A-40B, 41A-41C, 42A-42C, 43A-43C, 44A-44C, 45A-45C, 46A-46B, 47A-47C, 48A-48C, 49A-49C, 50A-50C, 51A-51C, 52A-52C, 53A-53B, 54A-54C, 55A-55C, 56A-56D, 57A-57C, 58A-58C, 59A-59C, 60A-60C, 61A-61C, 62A-62C, 63A-63C, 64A-64D, 65A-65C, 66A-66B, 67A-67B, 68A-68B, 69A-69B, 70A-70B, 71A-71B, 72A-72B, 73A-73B, 74A-74C, 75A-75B, 76A-76B, 77A-77B, 78A-78B, 79A-79B, 80A-80B, 81A-81B, 82A-82B, 83A-83B, 848A-84B, 85A-85B, 86A-86B, 87A-87B, 88A-88B, 89A-89B, 90A-90B, 91A-91B, 92A-92B, 93A-93C, 99A-99C, 95A-95C, 96A-96D, 97A-97C, 98A-98C, 99A-99C, 100A-100C, 101A-101C, 102A-102B, 103A-103C, 104A-104C, 105A-105B, 106A-106B, 107, 108A-108B, 109A-109B, 110A-110B, 111A-111B, 112A-112B, 113A-113B, 114A-114B, 115A-115B, 116A-116B, 117A-117B, 118A-118B, 119A-119B, 120A-120B, 121A-121B, 122A-122B, 123A-123B, 124A-124B, 125A-125B, 126A-126B, 127A-127B, 128A-128B, 129A-129B, 130A-130B, 131A-131B, 132A-132B, 133A-133B, 134A-134B, 135A-135B, 136A-136B, 137A-137B, 138A-138B, 139A-139B, 140A-140B, 141A-141B, 142A-142B, 143A-143B, 144A-144B, 145A-145B, 146A-146B, 147A-147B, 148A-148B, 149A-149B, 150A-150B, 151A-151B, 152A-152B, 153, 154A-154B, 155, 156, 157, 158, 159A-159B, 160, 161A-161B, 162, 163, 164A-164B, 165, 166, 167A-167B, 168, 169, 170, 171A-171B, 172, 173, 174A-174B, 175, 176, 177, 178, 179A-179B, 180A-180B, 181, 182, 183, 184, 185, 186A-186B, 187A-187B, 188A-188B, 189A-189B show data pertaining to examples 1-189, respectively.

FIG. 190 shows a representative 2D gel of proteins in elementary bodies.

FIG. 191 shows an alignment of sequences in five (six) proteins of the invention.

### EXAMPLES

The examples indicate C. pneumoniae proteins, together with evidence to support the view that the proteins are useful antigens for vaccine production and development or for diagnostic purposes. This evidence takes the form of:
Computer prediction based on sequence information from CWL029 strain (e.g. using the PSORT algorithm).
Data on recombinant expression and purification of the proteins cloned from IOL207 strain.
Western blots to demonstrate immunoreactivity in serum (typically a blot of an EB extract of C. pneumoniae strain FB/96 stained with mouse antiserum against the recombinant protein).
FACS analysis of C. pneumoniae bacteria or purified EBs to confirm accessibility of the antigen to the immune system (see also table III).
An indication if the protein was identified by MALDI-TOF from a 2D gel electrophoresis map of proteins from purified elementary bodies from strain FB/96. This confirms that the protein is expressed in vivo (see also table V).

Various tests can be used to assess the in vivo immunogenicity of the proteins identified in the examples. For example, the proteins can be expressed recombinantly and used to screen patient sera by immunoblot. A positive reaction between the protein and patient serum indicates that the patient has previously mounted an immune response to the protein in question ie. the protein is an immunogen. This method can also be used to identify immunodominant proteins.

The recombinant protein can also be conveniently used to prepare antibodies e.g. in a mouse. These can be used for direct confirmation that a protein is located on the cell-surface. Labelled antibody (e.g. fluorescent labelling for FACS) can be incubated with intact bacteria and the presence of label on the bacterial surface confirms the location of the protein.

In particular, the following methods (A) to (O) were used to express, purify and biochemically characterise the proteins of the invention:

Cloning of CPN ORFs for Expression in E. coli

ORFs of Chlamydia pneumoniae (Cpn) were cloned in such a way as to potentially obtain three different kind of proteins:
a) proteins having an hexa-histidine tag at the C-terminus (cpn-His)
b) proteins having a GST fusion partner at the N-terminus (Gst-cpn)
c) proteins having both hexa-histidine tag at the C-terminus and GST at the N-terminus (GST/His fusion; NH₂-GST-cpn-(His)₆-COOH)

The type a) proteins were obtained upon cloning in the pET21b+ (Novagen). The type b) and c) proteins were obtained upon cloning in modified pGEX-KG vectors [Guan & Dixon (1991) Anal. Biochem. 192:262]. For instance pGEX-KG was modified to obtain pGEX-NN, then by modifying pGEX-NN to obtain pGEX-NNH. The Gst-cpn and Gst-cpn-His proteins were obtained in pGEX-NN and pGEX-NNH respectively.

The modified versions of pGEX-KG vector were made with the aim of allowing the cloning of single amplification products in all three vectors after only one double restriction enzyme digestion and to minimise the presence of extraneous amino acids in the final recombinant proteins.

(A) Construction of pGEX-NN and pGEX-NNH Expression Vectors

Two couples of complementary oligodeoxyribonucleotides were synthesised using the DNA synthesiser ABI394 (Perkin Elmer) and the reagents from Cruachem (Glasgow, Scotland). Equimolar amounts of the oligo pairs (50 ng each oligo) were annealed in T4 DNA ligase buffer (New England Biolabs) for 10 min in a final volume of 50 μl and then were left to cool slowly at room temperature. With the described procedure he following DNA linkers were obtained:

```
gexNN linker (SEQ ID NO: 657):
NdeI NheI XmaI EcoRI NcoI SalI XhoI SacI NotI
GATCCCATATGGCTAGCCCGGGGAATTCGTCCATGGAGTGAGTCGACTGACTCGAGTGATCGAGCTCCTGAGCGGCCGCATGAA
        GGTATACCGATCGGGCCCCTTAAGCAGGTACCTCACTCAGCTGACTGAGCTCACTAGCTCGAGGACTCGCCGGCGTACTTTCGA
gexKNH linker (SEQ ID NO: 658):
          HindIII NotI XhoI --Hexa-Bistidine--
TCGACAAGCTTGCGGCCGCACTCGAGCATCACCATCACCATCACTGAT
        GTTCGAACGCCGGCGTGAGCACGTAGAGGTAGTGGTAGTGACTATCGA
```

The plasmid pGEX-KG was digested with BamHI and HindIII and 100 ng were ligated overnight at 16° C. to the linker gexNN with a molar ratio of 3:1 linker/plasmid using 200 units of T4 DNA ligase (New england Biolabs). After transformation of the ligation product in E. coli DH5, a clone containing the pGEX-NN plasmid, having the correct linker, was selected by means of restriction enzyme analysis and DNA sequencing.

The new plasmid pGEX-NN was digested with SalI and HindIII and ligated to the linker gexNNH. After transformation of the ligation product in E. coli DH5, a clone containing the pGEX-NNH plasmid, having the correct linker, was selected by means of restriction enzyme analysis and DNA sequencing.

(B) Chromosomal DNA Preparation

The chromosomal DNA of elementary bodies (EB) of C. pneumoniae strain 10L-207 was prepared by adding 1.5 ml of lysis buffer (10 mM Tris-HCl, 150 mM NaCl, 2 mM EDTA, 0.6% SDS, 100 μg/ml Proteinase K, pH 8) to 450 μl EB suspension (400.000/μl) and incubating overnight at 37° C. After sequential extraction with phenol, phenol-chloroform, and chloroform, the DNA was precipitated with 0.3 M sodium acetate, pH 5.2 and 2 volumes of absolute ethanol. The DNA pellet was washed with 70% ethanol. After solubilization with distilled water and treatment with 20 μg/ml RNAse A for 1 hour at RT, the DNA was extracted again with phenol-chloroform, alcohol precipitated and suspended with 300 μl 1 mM Tris-HCl pH 8.5. The DNA concentration was evaluated by measuring OD₂₆₀ of the sample.

(C) Oligonucleotide Design

Synthetic oligonucleotide primers were designed on the basis of the coding sequence of each ORF using the sequence of C. pneumoniae strain CWL029. Any predicted signal peptide were omitted, by deducing the 5′ end amplification primer sequence immediately downstream from the predicted leader sequence. For most ORFs, the 5′ tail of the primers (table I) included only one restriction enzyme recognition site (NdeI, or NheI, or SpeI depending on the gene's own restriction pattern); the 3′ primer tails (table I) included a XhoI or a NotI or a HindIII restriction site.

TABLE I
Oligonucleotide tails of the
primers used to amplify Cpn genes.

| 5′ tails | 3′ tails |
|---|---|
| NdeI | XhoI |
| 5′ GTGCGTCATATG 3′ | 5′ GCGTCTGAG 3′ |
| (SEQ ID NO: 659) | (SEQ ID NO: 660) |
| NheI | NotI |
| 5′ GTGCGTGCTAGC 3′ | 5′ ACTCGCTAGCGGCCGC 3′ |
| (SEQ ID NO: 661) | (SEQ ID NO: 662) |
| SpeI | HindIII |
| 5′ GTGCGTACTAGT 3′ | 5′ GCGTAAGCTT 3′ |
| (SEQ ID NO: 663) | (SEQ ID NO: 664) |

As well as containing the restriction enzyme recognition sequences, the primers included nucleotides which hybridized to the sequence to be amplified. The number of hybridizing nucleotides depended on the melting temperature of the primers which was determined as described [(Breslauer et al. (1986) PNAS USA 83:3746-50]. The average melting temperature of the selected oligos was 50-55° C. for the hybridizing region alone and 65-75° C. for the whole oligos. Table II shows the forward and reverse primers used for each amplification.

(D) Amplification

The standard PCR protocol was as follow: 50 ng genomic DNA were used as template in the presence of 0.2 μM each primer, 200 μM each dNTP, 1.5 mM MgCl₂, 1× PCR buffer minus Mg (Gibco-BRL), and 2 units of Taq DNA polymerase (Platinum Taq, Gibco-BRL) in a final volume of 100 μl. Each sample underwent a double-step amplification: the first 5 cycles were performed using as the hybridizing temperature the one of the oligos excluding the restriction enzyme tail, followed by 25 cycles performed according to the hybridization temperature of the whole length primers. The standard cycles were as follow:

$$denaturation \text{:} 94 ° C . , 2 min \\ \begin{matrix}denaturation \text{:} 94 ° C . , 30 seconds \\ hybridization \text{:} 51 ° C . , 50 seconds\end{matrix} \} 5 cycles \\ elongation \text{:} 72 ° C . , 1 min or 2 min and 40 sec \\ \begin{matrix}denaturation \text{:} 94 ° C . , 30 seconds \\ hybridization \text{:} 70 ° C . , 50 seconds\end{matrix} \} 25 cycles$$

$$elongation \text{:} 72 ° C . , 1 min or 2 min and 40 sec$$

$$72 ° C . , 7 min$$

$$4 ° C .$$

The elongation time was 1 min for ORFs shorter than 2000 bp, and 2 min and 40 seconds for ORFs longer than 2000 bp. The amplifications were performed using a Gene Amp PCR system 9600 (Perkin Elmer).

To check the amplification results, 4 μl of each PCR product was loaded onto 1-1.5 agarose gel and the size of amplified fragments compared with DNA molecular weight standards (DNA markers III or IX, Roche). The PCR products were loaded on agarose gel and after electrophoresis the right size bands were excised from the gel. The DNA was purified from the agarose using the Gel Extraction Kit (Qiagen) following the instruction of the manufacturer. The final elution volume of the DNA was 50 μl TE (10 mM Tris-HCl, 1 mM EDTA, pH 8). One μl of each purified DNA was loaded onto agarose gel to evaluate the yield.

(E) Digestion of PCR Fragments

One-two μg of purified PCR product were double digested overnight at 37° C. with the appropriate restriction enzymes (60 units of each enzyme) using the appropriate restriction buffer in 100 μl final volume. The restriction enzymes and the digestion buffers were from New England Biolabs. After purification of the digested DNA (PCR purification Kit, Qiagen) and elution with 30 μl TE, 1 μl was subjected to agarose gel electrophoresis to evaluate the yield in comparison to titrated molecular weight standards (DNA markers III or IX, Roche).

(F) Digestion of the Cloning Vectors (pET21b+, pGEX-NN, and pGEX-NNH)

10 μg of plasmid was double digested with 100 units of each restriction enzyme in 400 μl reaction volume in the presence of appropriate buffer by overnight incubation at 37° C. After electrophoresis on a 1% agarose gel, the band corresponding to the digested vector was purified from the gel using the Qiagen Qiaex II Gel Extraction Kit and the DNA was eluted with 50 μl TE. The DNA concentration was evaluated by measuring OD₂₆₀ of the sample.

(G) Cloning

75 ng of the appropriately digested and purified vectors and the digested and purified fragments corresponding to each ORF, were ligated in final volumes of 10-20 μl with a molar ratio of 1:1 fragment/vector, using 400 units T4 DNA ligase (New England Biolabs) in the presence of the buffer supplied by the manufacturer. The reactions were incubated overnight at 16° C.

Transformation in E coli DH5 competent cells was performed as follow: the ligation reaction was mixed with 200 μl of competent DH5 cells and incubated on ice for 30 min and then at 42° C. for 90 seconds. After cooling on ice, 0.8 ml LB was added and the cells were incubated for 45 min at 37° C. under shaking. 100 and 900 μl of cell suspensions were plated on separate plates of agar LB 100 μg/ml Ampicillin and the plates were incubated overnight at 37° C. The screening of the transformants was done by growing randomly chosen clones in 6 ml LB 100 μg/ml Ampicillin, by extracting the DNA using the Qiagen Qiaprep Spin Miniprep Kit following the manufacturer instructions, and by digesting 2 μl of plasmid minipreparation with the restriction enzymes specific for the restriction cloning sites. After agarose gel electrophoresis of the digested plasmid mini-preparations, positive clones were chosen on the basis of the correct size of the restriction fragments, as evaluated by comparison with appropriate molecular weight markers (DNA markers III or IX, Roche).

(H) Expression

1 μl of each right plasmid mini-preparation was transformed in 200 μl of competent E. coli strain suitable for expression of the recombinant protein. All pET21b+ recombinant plasmids were transformed in BL21 DE3 (Novagen) E. coli cells, whilst all pGEX-NN and all pGEX-NNH recombinant plasmids were transformed in BL21 cells (Novagen). After plating transformation mixtures on LB/Amp agar plates and incubation overnight at 37° C., single colonies were inoculated in 3 ml LB 100 μg/ml Ampicillin and grown at 37° C. overnight. 70 μl of the overnight culture was inoculated in 2 ml LB/Amp and grown at 37° C. until OD₆₀₀ of the pET clones reached the 0.4-0.8 value or until OD₆₀₀ of the pGEX clones reached the 0.8-1 value. Protein expression was then induced by adding IPTG (Isopropil β-D thio-galacto-piranoside) to the mini-cultures. pET clones were induced using 1 mM IPTG, whilst pGEX clones were induced using 0.2 mM IPTG. After 3 hours incubation at 37° C. the final OD₆₀₀ was checked and the cultures were cooled on ice. After centrifugation of 0.5 ml culture, the cell pellet was suspended in 50 μl of protein Loading Sample Buffer (60 mM TRIS-HCl pH 6.8, 5% w/v SDS, 10% v/v glycerin, 0.1% w/v Bromophenol Blue, 100 mM DTT) and incubated at 100° C. for 5 min. A volume of boiled sample corresponding to 0.1 OD₆₀₀ culture was analysed by SDS-PAGE and Coomassie Blue staining to verify the presence of induced protein band.

Purification of the Recombinant Proteins

Single colonies were inoculated in 25 ml LB 100 μg/ml Ampicillin and grown at 37° C. overnight. The overnight culture was inoculated in 500 ml LB/Amp and grown under shaking at 25° C. until OD₆₀₀ 0.4-0.8 value for the pET clones, or until OD₆₀₀ 0.8-1 value for the pGEX clones. Protein expression was then induced by adding IPTG to the cultures. pET clones were induced using 1 mM IPTG, whilst pGEX clones were induced using 0.2 mM IPTG. After 4 hours incubation at 25° C. the final OD₆₀₀ was checked and the cultures were cooled on ice. After centrifugation at 6000 rpm (JA10 rotor, Beckman), the cell pellet was processed for purification or frozen at −20° C.

(I) Procedure for the Purification of Soluble His-Tagged Proteins from E. coli
1. Transfer the pellets from −20° C. to ice bath and reconstitute with 10 ml 50 mM NaHPO₄ buffer, 300 mM NaCl, pH 8.0, pass in 40-50 ml centrifugation tubes and break the cells as per the following outline:
2. Break the pellets in the French Press performing three passages with in-line washing.
3. Centrifuge at about 30-40000'g per 15-20 min. If possible use rotor JA 25.50 (21000 rpm, 15 min.) or JA-20 (18000 rpm, 15 min.)
4. Equilibrate the Poly-Prep columns with 1 ml Fast Flow Chelating Sepharose resin with 50 mM phosphate buffer, 300 mM NaCl, pH 8.0.
5. Store the centrifugation pellet at −20° C., and load the supernatant in the columns.
6. Collect the flow through.
7. Wash the columns with 10 ml (2 ml+2 ml+4 ml) 50 mM phosphate buffer, 300 mM NaCl, pH 8.0.
8. Wash again with 10 ml 20 mM imidazole buffer, 50 mM phosphate, 300 mM NaCl, pH 8.0.
9. Elute the proteins bound to the columns with 4.5 ml (1.5 ml+1.5 ml+1.5 ml) 250 mM imidazole buffer, 50 mM phosphate, 300 mM NaCl, pH 8.0 and collect the 3 corresponding fractions of ˜1.5 ml each. Add to each tube 15 μl DTT 200 mM (final concentration 2 mM)
10. Measure the protein concentration of the first two fractions with the Bradford method, collect a 10 μg aliquot of proteins from each sample and analyse by SDS-PAGE. (N.B.: should the sample be too diluted, load 21 μl+7 μl loading buffer).
11. Store the collected fractions at +4° C. while waiting for the results of the SDS-PAGE analysis.
12. For immunization prepare 4-5 aliquots of 100 μg each in 0.5 ml in 40% glycerol. The dilution buffer is the above elution buffer, plus 2 mM DTT. Store the aliquots at −20° C. until immunization.

(J) Purification of His-Tagged Proteins from Inclusion Bodies

Purifications were carried out essentially according the following protocol:
1. Bacteria are collected from 500 ml cultures by centrifugation. If required store bacterial pellets at −20° C. For extraction, resuspend each bacterial pellet in 10 ml 50 mM TRIS-HCl buffer, pH 8.5 on an ice bath.
2. Disrupt the resuspended bacteria with a French Press, performing two passages.
3. Centrifuge at 35000×g for 15 min and collect the pellets. Use a Beckman rotor JA 25.50 (21000 rpm, 15 min.) or JA-20 (18000 rpm, 15 min.).
4. Dissolve the centrifugation pellets with 50 mM TRIS-HCl, 1 mM TCEP {Tris(2-carboxyethyl)-phosphine hydrochloride, Pierce}, 6M guanidium chloride, pH 8.5. Stir for ˜10 min. with a magnetic bar.
5. Centrifuge as described above, and collect the supernatant.
6. Prepare an adequate number of Poly-Prep (Bio-Rad) columns containing 1 ml of Fast Flow Chelating Sepharose (Pharmacia) saturated with Nichel according to manufacturer recommendations. Wash the columns twice with 5 ml of H₂0 and equilibrate with 50 mM TRIS-HCl, 1 mM TCEP, 6M guanidinium chloride, pH 8.5.
7. Load the supernatants from step 5 onto the columns, and wash with 5 ml of 50 mM TRIS-Hcl buffer, 1 mM TCEP, 6M urea, pH 8.5
8. Wash the columns with 10 ml of 20 mM imidazole, 50 mM TRIS-HCl, 6M urea, 1 mM TCEP, pH 8.5. Collect and set aside the first 5 ml for possible further controls.
9. Elute the proteins bound to the columns with 4.5 ml of a buffer containing 250 mM imidazole, 50 mM TRIS-HCl, 6M urea, 1 mM TCEP, pH 8.5. Add the elution buffer in three 1.5 ml aliquots, and collect the corresponding 3 fractions. Add to each fraction 15 μl DTT (final concentration 2 mM).
10. Measure eluted protein concentration with the Bradford method, and analyze aliquots of ca 10 μg of protein by SDS-PAGE.
11. Store proteins at −20° C. in 40% (v/v) glycerol, 50 mM TRIS-HCl, 2M urea, 0.5 M arginine, 2 mM DTT, 0.3 mM TCEP, 83.3 mM imidazole, pH 8.5

(K) Procedure for the Purification of GST-Fusion Proteins from E. coli
1. Transfer the bacterial pellets from −20° C. to an ice bath and resuspend with 7.5 ml PBS, pH 7.4 to which a mixture of protease inhibitors (CØMPLETE™—Boehringer Mannheim, 1 tablet every 25 ml of buffer) has been added. Transfer to 40-50 ml centrifugation tubes and sonicate according to the following procedure:
   a) Position the probe at about 0.5 cm from the bottom of the tube
   b) Block the tube with the clamp
   c) Dip the tube in an ice bath
   d) Set the sonicator as follows: Timer→Hold, Duty Cycle→55, Out. Control→6.
   e) perform 5 cycles of 10 impulses at a time lapse of 1 minute (i.e. one cycle=10 impulses+˜45″ hold; b. 10 impulses+˜45″ hold; c. 10 impulses+˜45″ hold; d. 10 impulses+˜45″ hold; e. 10 impulses+˜45″ hold)
2. Centrifuge at about 30-40000×g for 15-20 min. E.g.: use rotor Beckman JA 25.50 at 21000 rpm, for 15 min.
3. Store the centrifugation pellets at −20° C., and load the supernatants on the chromatography columns, as follows
4. Equilibrate the Poly-Prep (Bio-Rad) columns with 0.5 ml (≅1 ml suspension) of Glutathione-Sepharose 4B resin, wash with 2 ml (1+1) H₂O, and then with 10 ml (2+4+4) PBS, pH 7.4,
5. Load the supernatants on the columns and discard the flow through.
6. Wash the columns with 10 ml (2+4+4) PBS, pH 7.4.
7. Elute the proteins bound to the columns with 4.5 ml of 50 mM TRIS buffer, 10 mM reduced glutathione, pH 8.0, adding 1.5 ml+1.5 ml+1.5 ml and collecting the respective 3 fractions of ˜1.5 ml each.
8. Measure the protein concentration of the first two fractions with the Bradford method, analyse a 10 μg aliquot of proteins from each sample by SDS-PAGE. (N.B.: if the sample is too diluted load 21 μl (+7 μl loading buffer).
9. Store the collected fractions at +4° C. while waiting for the results of the SDS-PAGE analysis.
10. For each protein destined to the immunization prepare 4-5 aliquots of 100 μg each in 0.5 ml of 40% glycerol. The dilution buffer is 50 mM TRIS.HCl, 2 mM DTT, pH 8.0. Store the aliquots at −20° C. until immunization.

Serology

(L) Protocol of Immunization

1. Groups of four CD1 female mice aged between 6 and 7 weeks were immunized with 20 μg of recombinant protein resuspended in 100 μl.

2. Four mice for each group received 3 doses with a 14 days interval schedule.

3. Immunization was performed through intra-peritoneal injection of the protein with an equal volume of Complete Freund's Adjuvant (CFA) for the first dose and Incomplete Freund's Adjuvant (IFA) for the following two doses.

4. Sera were collected before each immunization. Mice were sacrified 14 days after the third immunization and the collected sera were pooled and stored at −20° C.

(M) Western Blot Analysis of Cpn Elementary Body Proteins with Mouse Sera

Aliquots of elementary bodies containing approximately 4 μg of proteins, mixed with SDS loading buffer (1×: 60 mM TRIS-HCl pH 6.8, 5% w/v SDS, 10% v/v glycerin, 0.1% Bromophenol Blue, 100 mM DTT) and boiled 5 minutes at 95° C., were loaded on a 12% SDS-PAGE gel. The gel was run using a SDS-PAGE running buffer containing 250 mM TRIS, 2.5 mM Glycine and 0.1% SDS. The gel was electroblotted onto nitrocellulose membrane at 200 mA for 30 minutes. The membrane was blocked for 30 minutes with PBS, 3% skimmed milk powder and incubated O/N at 4° C. with the appropriate dilution ( 1/100) of the sera. After washing twice with PBS+0.1% Tween (Sigma) the membrane was incubated for 2 hours with peroxidase-conjugated secondary anti-mouse antibody (Sigma) diluted 1:3000. The nitrocellulose was washed twice for 10 minutes with PBS+0.1% Tween-20 and once with PBS and thereafter developed by Opti-4CN Substrate Kit (Biorad).

Lanes shown in Western blots are: (P)=pre-immune control serum; (I)=immune serum.

(N) FACS Analysis of Chlamydia pneumoniae Elementary Bodies with Mouse Sera
1. 2×10⁵ Elementary Bodies (EB)/well were washed with 200 μl of PBS-0.1% BSA in a 96 wells U bottom plate and centrifuged for 10 min. at 1200 rpm, at 4° C.
2. The supernatant was discarded and the E.B. resuspended in 10 μl of PBS-0.1% BSA.
3. 10 μl mouse sera diluted in PBS-0.1% BSA were added to the E.B. suspention to a final dilution of 1:400, and incubated on ice for 30 min.
4. EB were washed by adding 180 μl PBS-01% BSA and centrifuged for lOmin. at 1200 rpm, 4° C.
5. The supernatant was discarded and the E.B. resuspended in 10 l of PBS-01% BSA.
6. 10 μl of a goat anti-mouse IgG, F(ab′)₂ fragment specific-R-Phycoerythrin-conjugated (Jackson Immunoresearch Laboratories Inc., cat.N°115-116-072) was added to the EB suspension to a final dilution of 1:100, and incubated on ice for 30 min. in the dark.
7. EB were washed by adding 180 μl PBS-0.1% BSA and centrifuged for 10 min. at 1200 rpm, 4° C.
8. The supernatant was discarded and the E.B. resuspended in 150 μl of PBS-01% BSA.
9. E.B. suspension was passed through a cytometric chamber of a FACS Calibur (Becton Dikinson, Mountain View, Calif. USA) and 10.000 events were acquired.
10. Data were analysed using Cell Quest Software (Becton Dikinson, Mountain View, Calif. USA) by drawing a morphological dot plot (using forward and side scatter parameters) on E.B. signals. An histogram plot was then created on FL2 intensity of fluorescence log scale recalling the morphological region of EB.

NB: the results of FACS depend not only on the extent of accessibility of the native antigens but also on the quality of the antibodies elicited by the recombinant antigens, which may have structures with a variable degree of correct folding as compared with the native protein structures. Therefore, even if a FACS assay appears negative this does not necessarily mean that the protein is not abundant or accessible on the surface. PorB antigen, for instance, gave negative results in FACS but is a surface-exposed neutralising antigen [Kubo & Stephens (2000) Mol. Microbiol. 38:772-780].

(O) Mass Spectrometry Analysis of Two-Dimensional Electrophoretic Protein Maps

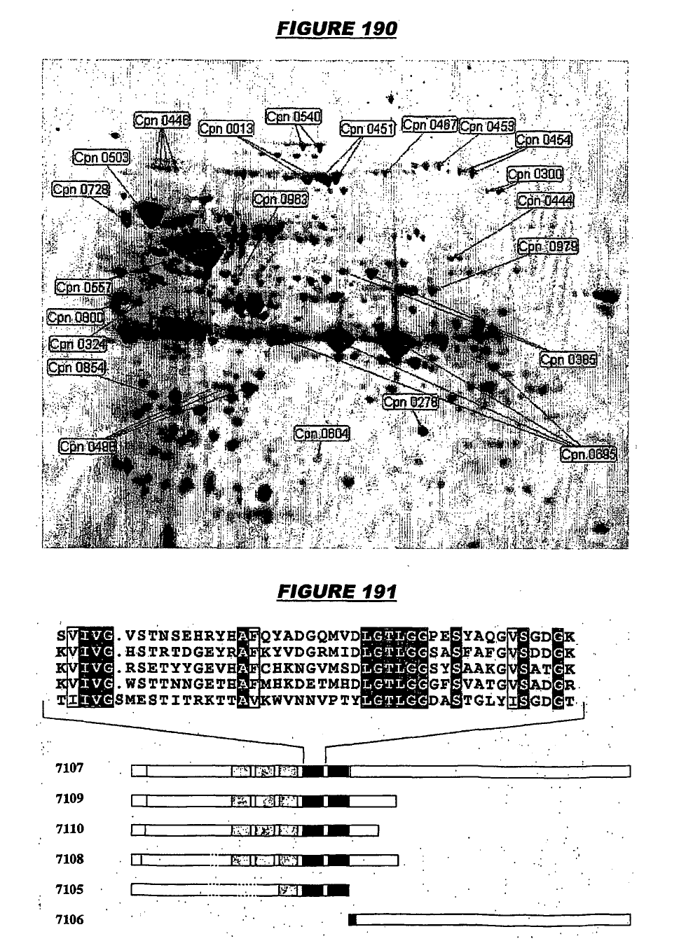

Gradient purified EBs from strain FB/96 were solubilized at a final concentration of 5.5 mg/ml with immobiline rehydration buffer (7M urea, 2M thiourea, 2% (w/v) CHAPS, 2% (w/v) ASB 14 [Chevallet et al. (1998) Electrophor. 19:1901-9], 2% (v/v) C.A 3-10NL (Amersham Pharmacia Biotech), 2 mM tributyl phosphine, 65 mM DTT). Samples (250 μg protein) were adsorbed overnight on Immobiline DryStrips (7 cm, pH 3-10 non linear). Electrofocusing was performed in a IPGphor Isoelectric Focusing Unit (Amersham Pharmacia Biotech). Before PAGE separation, the focused strips were incubated in 4M urea, 2M thiourea, 30% (v/v) glycerol, 2% (w/v) SDS, 5 mM tributyl phosphine 2.5% (w/v) acrylamide, 50 mM Tris-HCl pH 8.8, as described [Herbert et al. (1998) Electrophor. 19:845-51]. SDS-PAGE was performed on linear 9-16% acrylamide gradients. Gels were stained with colloidal Coomassie (Novex, San Diego) [Doherty et al. (1998) Electrophor. 19:355-63]. Stained gels were scanned with a Personal Densitometer SI (Molecular Dynamics) at 8 bits and 50 μm per pixel. Map images were annotated with the software Image Master 2D Elite, version 3.10 (Amersham Pharmacia Biotech). Protein spots were excised from the gel, using an Ettan Spot picker (Amersham Pharmacia Biotech), and dried in a vacuum centrifuge. In-gel digestion of samples for mass spectrometry and extraction of peptides were performed as described by Wilm et al. [Nature (1996) 379:466-9]. Samples were desalted with a ZIP TIP (Millipore), eluted with a saturated solution of alpha-cyano-4-hydroxycinnamic acid in 50% acetonitrile, 0.1% TFA and directly loaded onto a SCOUT 381 multiprobe plate (Bruker). Spectra were acquired on a Bruker Biflex II MALDI-TOF. Spectra were calibrated using a combination of known standard peptides, located in spots adjacent to the samples. Resulting values for monoisotopic peaks were used for database searches using the computer program Mascot (matrixscience.com). All searches were performed using an error of 200-500 ppm as constraint. A representative gel is shown in FIG. 190.

### Example 1

The following C. pneumoniae protein (PID 4376552) was expressed <SEQ ID 1; cp6552>:

```
    1 MKKKLSLLVG LIFVLSSCHK EDAQNKIRIV ASPTPHAELL ESLQEEAKDL
  51 GIKLKILPVD DYRIPNRLLL DKQVDANYFQ HQAFLDDECE RYDCKGELVV
101 IAKVHLEPQA IYSKKHSSLE RLKSQKKLTI AIPVDRTNAQ RALHLLEECG
151 LIVCKGPANL NMTAKDVCGK ENRSINILEV SAPLLVGSLP DVDAAVIPGN
201 FAIAANLSPK KDSLCLEDLS VSKYTNLVVI RSEDVGSPKM IKLQKLFQSP
251 SVQHFFDTKY HGNILTMTQD NG*
```

A predicted signal peptide is highlighted.

The cp6552 nucleotide sequence <SEQ ID 2> is:

```
    1 ATGAAAAAAA AATTATCATT ACTTGTAGGT TTAATTTTTG TTTTGAGTTC
  51 TTGCCATAAG GAAGATGCTC AGAATAAAAT ACGTATTGTA GCCAGTCCGA
101 CACCTCATGC GGAATTATTG GAGAGTTTAC AGGAAGAGGC TAAAGATCTT
151 GGAATCAAGC TGAAAATACT TCCAGTAGAT GATTATCGTA TTCCTAATCG
201 TTTGCTTTTG GATAAACAAG TAGATGCAAA TTACTTTCAA CATCAAGCTT
251 TTCTTGATGA CGAATGCGAG CGTTATGATT GTAAGGGTGA ATTAGTTGTT
301 ATCGCTAAAG TTCATTTGGA ACCTCAAGCA ATTTATTCTA AGAAACATTC
351 TTCTTTAGAG CGCTTAAAAA GCCAGAAGAA ACTGACTATA GCGATTCCTG
401 TGGATCGTAC GAATGCTCAG CGTGCTCTAC ACTTGTTAGA AGAGTGCGGA
451 CTCATTGTTT GCAAAGGGCC TGCTAATTTA AATATGACAG CTAAAGATGT
501 CTGTGGGAAA GAAAATAGAA GTATCAACAT ATTAGAGGTG TCAGCTCCTC
551 TTCTTGTCGG ATCTCTTCCT GACGTTGATG CTGCTGTCAT TCCTGGAAAT
601 TTTGCTATAG CAGCAAACCT TTCTCCAAAG AAAGATAGTC TTTGTTTAGA
651 GGATCTTTCG GTATCTAAGT ATACAAACCT TGTTGTCATT CGTTCTGAAG
701 ACGTAGGTTC TCCTAAAATG ATAAAATTAC AGAAGCTGTT TCAATCTCCT
751 TCTGTACAAC ATTTTTTTGA TACAAAATAT CATGGGAATA TTTTGACAAT
801 GACTCAAGAC AATGGTTAG
```

The PSORT algorithm predicts an inner membrane location (0.127).

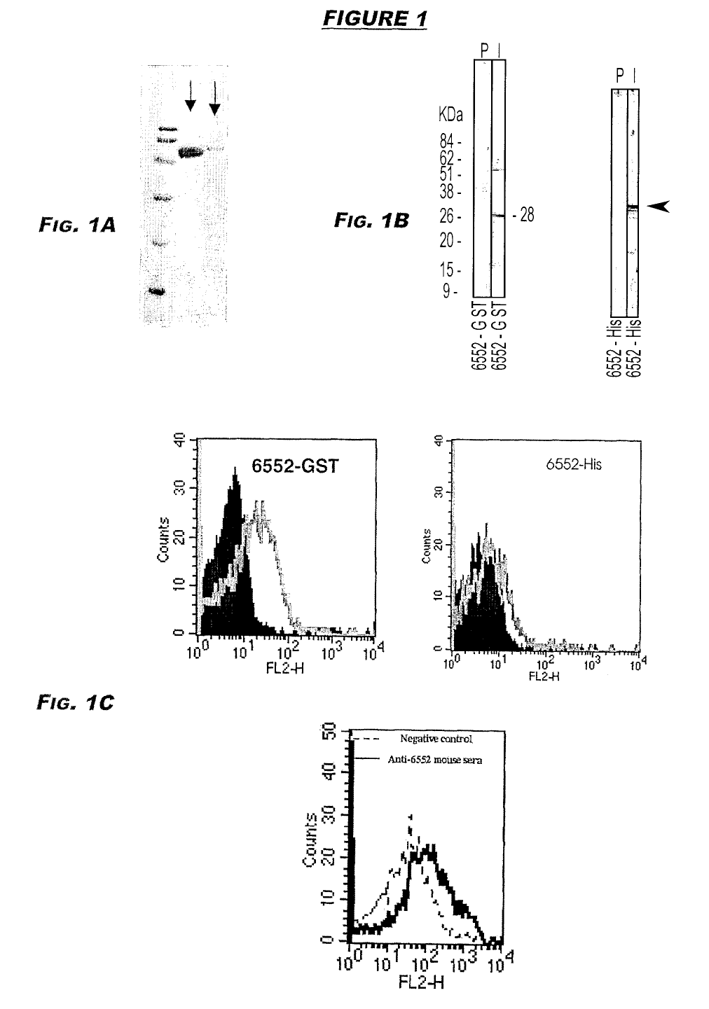

The protein was expressed in E. coli and purified as a his-tag product, as shown in FIG. 1A, and also as a GST-fusion. The recombinant protein was used to immunize mice, whose sera were used in a Western blot (FIG. 1B) and for FACS analysis (FIG. 1C).

The cp6552 protein was also identified in the 2D-PAGE experiment (Cpn0278).

These experiments show that cp6552 is a surface-exposed and immunoaccessible protein, and that it is a useful immunogen. These properties are not evident from the sequence alone.

### Example 2

The following C. pneumoniae protein (PID 4376736) was expressed <SEQ ID 3; cp6736>:

```
    1 MKTSIRKFLI STTLAPCFAS TAFTVEVIMP SENFDGSSGK IFPYTTLSDP
  51 RGTLCIFSGD LYIANLDNAI SRTSSSCFSN RAGALQILGK GGVFSFLNIR
101 SSADGAAISS VITQNPELCP LSFSGFSQMI FDNCESLTSD TSASNVIPHA
151 SAIYATTPML FTNNDSILFQ YNRSAGFGAA IRGTSITIEN TKKSLLFNGN
201 GSISNGGALT GSAAINLINN SAAVIFSTNA TGIYGGAIYL TGGSMLTSGN
251 LSGVLFVNNS SRSGGAIYAN GNVTFSNNSD LTFQNNTASP QNSLPAPTPP
301 PTPPAVTPLL GYGGAIFCTP PATPPPTGVS LTISGENSVT FLENIASEQG
351 GALYGKKISI DSNKSITFLG NTAGKGGAIA IPESGELSLS ANQGDILFNK
401 NLSITSGTPT RNSIHFGKDA KFATLGATQG YTLYFYDPIT SDDLSAASAA
451 ATVVVNPKAS ADGAYSGTIV FSGETLTATE AATPANATST LNQKLELEGG
501 TLALRNGATL NVHNFTQDEK SVVIMDAGTT LATTNGANNT DGAITLNKLV
551 INLDSLDGTK AAVVNVQSTN GALTISGTLG LVKNSQDCCD NHGMFNKDLQ
601 QVPILELKAT SNTVTTTDFS LGTNGYQQSP YGYQGTWEFT IDTTTHTVTG
651 NWKKTGYLPH PERLAPLIPN SLWANVIDLR AVSQASAADG EDVPGKQLSI
701 TGITNFFHAN HTGDARSYRH MGGGYLINTY TRITPDAALS LGFGQLFTKS
751 KDYLVGHGHS NVYFATVYSN ITKSLFGSSR FFSGGTSRVT YSRSNEKVKT
801 SYTKLPKGRC SWSNNCWLGE LEGNLPITLS SRILNLKQII PFVKAEVAYA
851 THGGIQENTP EGRIFGHGHL LNVAVPVGVR FGKNSHNRPD FYTIIVAYAP
901 DVYRHNPDCD TTLPINGATW TSIGNNLTRS TLLVQASSHT SVNDVLEIFG
951 HCGCDIRRTS RQYTLDIGSK LRF*
```

A predicted signal peptide is highlighted.

The cp6736 nucleotide sequence <SEQ ID 4> is:

```
      1 ATGAAAACGT CTATTCGTAA GTTCTTAATT TCTACCACAC TGGCGCCATG
    51 TTTTGCTTCA ACAGCGTTTA CTGTAGAAGT TATCATGCCT TCCGAGAACT
  101 TTGATGGATC GAGTGGGAAG ATTTTTCCTT ACACAACACT TTCTGATCCT
  151 AGAGGGACAC TCTGTATTTT TTCAGGGGAT CTCTACATTG CGAATCTTGA
  201 TAATGCCATA TCCAGAACCT CTTCCAGTTG CTTTAGCAAT AGGGCGGGAG
  251 CACTACAAAT CTTAGGAAAA GGTGGGGTTT TCTCCTTCTT AAATATCCGT
  301 TCTTCAGCTG ACGGAGCCGC GATTAGTAGT GTAATCACCC AAAATCCTGA
  351 ACTATGTCCC TTGAGTTTTT CAGGATTTAG TCAGATGATC TTCGATAACT
  401 GTGAATCTTT GACTTCAGAT ACCTCAGCGA GTAATGTCAT ACCTCACGCA
  451 TCGGCGATTT ACGCTACAAC GCCCATGCTC TTTACAAACA ATGACTCCAT
  501 ACTATTCCAA TACAACCGTT CTGCAGGATT TGGAGCTGCC ATTCGAGGCA
  551 CAAGCATCAC AATAGAAAAT ACGAAAAAGA GCCTTCTCTT TAATGGTAAT
  601 GGATCCATCT CTAATGGAGG GGCCCTCACG GGATCTGCAG CGATCAACCT
  651 CATCAACAAT AGCGCTCCTG TGATTTTCTC AACGAATGCT ACAGGGATCT
  701 ATGGTGGGGC TATTTACCTT ACCGGAGGAT CTATGCTCAC CTCTGGGAAC
  751 CTCTCAGGAG TCTTGTTCGT TAATAATAGC TCGCGCTCAG GAGGCGCTAT
  801 CTATGCTAAC GGAAATGTCA CATTTTCTAA TAACAGCGAC CTGACTTTCC
  851 AAAACAATAC AGCATCTCCA CAAAACTCCT TACCTGCACC TACACCTCCA
  901 CCTACACCAC CAGCAGTCAC TCCTTTGTTA GGATATGGAG GCGCCATCTT
  951 CTGTACTCCT CCAGCTACCC CCCCACCAAC AGGTGTTAGC CTGACTATAT
1001 CTGGAGAAAA CAGCGTTACA TTCCTAGAAA ACATTGCCTC CGAACAAGGA
1051 GGAGCCCTCT ATGGCAAAAA GATCTCTATA GATTCTAATA AATCTACAAT
1101 ATTTCTTGGA AATACAGCTG GAAAAGGAGG CGCTATTGCT ATTCCCGAAT
1151 CTGGGGAGCT CTCTCTATCC GCAAATCAAG GTGATATCCT CTTTAACAAG
1201 AACCTCAGCA TCACTAGTGG GACACCTACT CGCAATAGTA TTCACTTCGG
1251 AAAAGATGCC AAGTTTGCCA CTCTAGGAGC TACGCAAGGC TATACCCTAT
1301 ACTTCTATGA TCCGATTACA TCTGATGATT TATCTGCTGC ATCCGCAGCC
1351 GCTACTGTGG TCGTCAATCC CAAAGCCAGT GCAGATGGTG CGTATTCAGG
1401 GACTATTGTC TTTTCAGGAG AAACCCTCAC TGCTACCGAA GCAGCAACCC
1451 CTGCAAATGC TACATCTACA TTAAACCAAA AGCTAGAACT TGAAGGCGGT
1501 ACTCTCGCTT TAAGAAACGG TGCTACCTTA AATGTTCATA ACTTCACGCA
1551 AGATGAAAAG TCCGTCGTCA TCATGGATGC AGGGACCACA TTAGCAACTA
1601 CAAATGGAGC TAATAATACT GACGGTGCTA TCACCTTAAA CAAGCTTGTA
1651 ATCAATCTGG ATTCTTTGGA TGGCACTAAA GCGGCTGTCG TTAATGTGCA
1701 GAGTACCAAT GGAGCTCTCA CTATATCCGG AACTTTAGGA CTTGTGAAAA
1751 ACTCTCAAGA TTGCTGTGAC AACCACGGGA TGTTTAATAA AGATTTACAG
1801 CAAGTTCCGA TTTTAGAACT CAAAGCGACT TCAAATACTG TAACCACTAC
1851 GGACTTCAGT CTCGGCACAA ACGGCTATCA GCAATCTCCC TATGGGTATC
1901 AAGGAACTTG GGAGTTTACC ATAGACACGA CAACCCATAC GGTCACAGGA
1951 AATTGGAAAA AAACCGGTTA TCTTCCTCAT CCGGAGCGTC TTGCTCCCCT
2001 CATTCCTAAT AGCCTATGGG CAAACGTCAT AGATTTACGA GCTGTAAGTC
2051 AAGCGTCAGC AGCTGATGGC GAAGATGTCC CTGGGAAGCA ACTGAGCATC
2101 ACAGGAATTA CAAATTTCTT CCATGCGAAT CATACCGGTG ATGCACGCAG
2151 CTACCGCCAT ATGGGTGGAG GCTACCTCAT CAATACCTAC ACACGCATCA
2201 CTCCAGATGC TGCGTTAAGT CTAGGTTTTG GACAGCTGTT TACAAAATCT
2251 AAGGATTACC TCGTAGGTCA CGGTCATTCT AACGTTTATT TCGCTACAGT
2301 ATACTCTAAC ATCACCAAGT CTCTGTTTGG ATCATCGAGA TTCTTCTCAG
2351 GAGGCACTTC TCGAGTTACC TATAGCCGTA GCAATGAGAA AGTAAAGACT
2401 TCATATACAA AATTGCCTAA AGGGCGCTGC TCTTGGAGTA ACAATTGCTG
2451 GTTAGGAGAA CTCGAAGGGA ACCTTCCCAT CACTCTCTCT TCTCGCATCT
2501 TAAACCTCAA GCAGATCATT CCCTTTGTAA AAGCTGAAGT TGCTTACGCG
2551 ACTCATGGGG GCATCCAAGA AAATACCCCC GAGGGGAGGA TTTTTGGACA
2601 CGGTCATCTA CTCAACGTTG CAGTTCCCGT AGGCGTCCGC TTTGGTAAAA
2651 ATTCTCATAA TCGACCAGAT TTTTACACTA TAATCGTAGC CTATGCTCCT
2701 GATGTCTATC GTCACAATCC TGATTGCGAT ACGACATTAC CTATTAATGG
2751 AGCTACGTGG ACCTCTATAG GGAATAATCT AACCAGAAGT ACTTTGCTAG
2801 TACAAGCATC CAGCCATACT TCAGTAAATG ATGTTCTAGA GATCTTCGGG
2851 CACTGTGGAT GTGATATTCG CAGAACCTCC CGTCAATATA CTCTAGATAT
2901 AGGAAGCAAA TTACGATTTT AA
```

The PSORT algorithm predicts an outer membrane location (0.917).

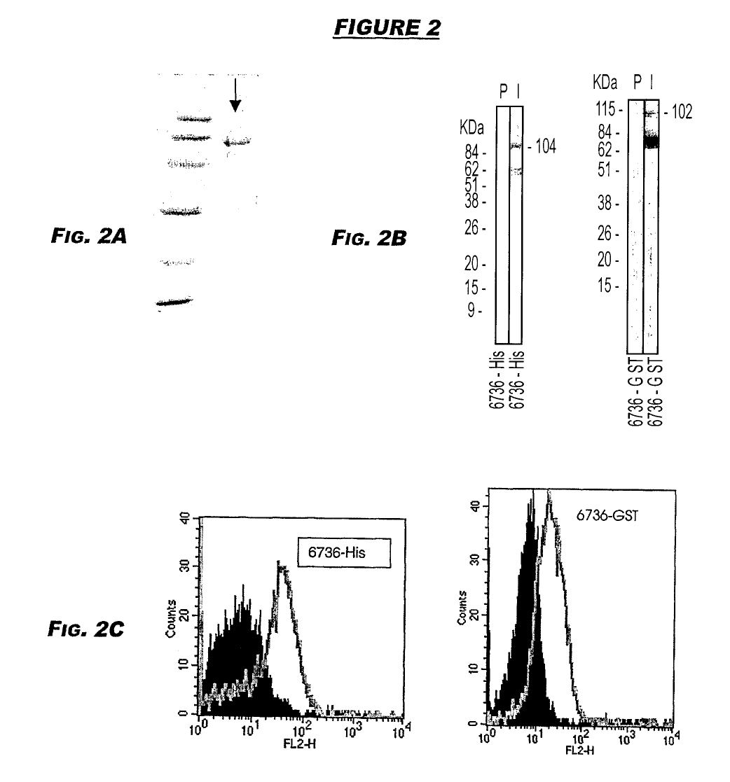

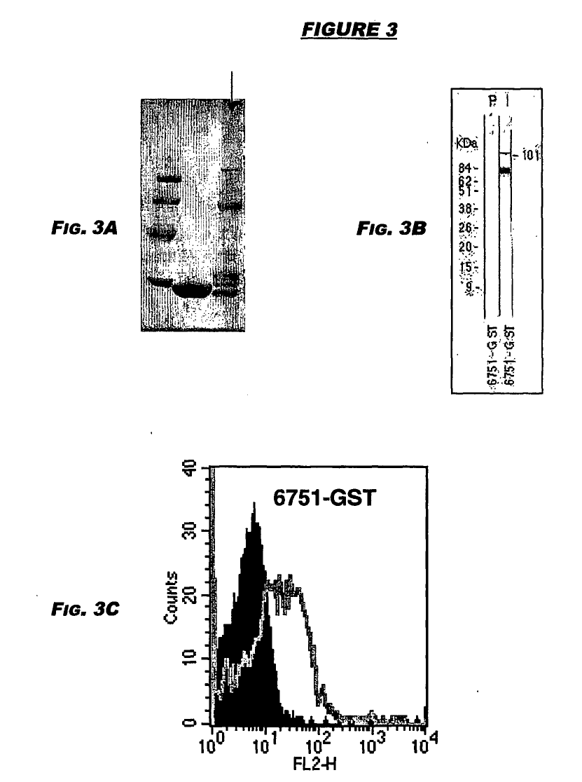

The protein was expressed in E. coli and purified as a his-tag product, as shown in FIG. 2A, and also as a GST-fusion. Both proteins were used to immunize mice, whose sera were used in a Western blot (FIG. 2B) and for FACS analysis (FIG. 2C).

The cp6736 protein was also identified in the 2D-PAGE experiment (Cpn0453) and showed good cross-reactivity with human sera, including sera from patients with pneumonitis.

These experiments show that cp6736 is a surface-exposed and immunoaccessible protein, and that it is a useful immunogen. These properties are not evident from the sequence alone.

### Example 3

The following C. pneumoniae protein (PID 4376751) was expressed <SEQ ID 5; cp6751>:

```
    1 MRFFCFGMLL PFTFVLANEG LQLPLETYIT LSPEYQAAPQ VGFTHNQNQD
  51 LAIVGNHNDF ILDYKYYRSN GGALTCKNLL ISENIGNVFF EKNVCPNSGG
101 AIYAAQNCTI SKNQNYAFTT NLVSDNPTAT AGSLLGGALF ATNCSITNNL
151 GQGTFVDNLA LNKGGALYTE TNLSIKDNKG PIIIKQNRAL NSDSLGGGIY
201 SGNSLNIEGN SGAIQITSNS SGSGGGIFST QTLTISSNKK LIEISENSAF
251 ANNYGSNFNP GGGGLTTTFC TILNNREGVL FNNNQSQSNG GAIHAKSIII
301 KENGPVYFLN NTATRGGALL NLSAGSGNGS FILSADNGDI IFNNNTASKH
351 ALNEPYRNAI HSTPNMNLQI GARPGYRVLF YDPIEHELPS SEETLENFET
401 GHTGTVLFSG EHVHQNFTDE MNFFSYLRNT SELRQGVLAV EDGAGLACYK
451 FFQRGGTLLL GQGAVITTAG TIPTPSSTPT TVGSTITLNH TAIDLESILS
501 FQAQAPKIWI YPTKTGSTYT EDSNPTITIS GTLTLRNSNN EDPYDSLDLS
551 HSLEKVPLLY IVDVAAQKIN SSQLDLSTLN SGEHYGYQGI WSTYWVETTT
601 ITNPTSLLGA NTKHKLLYAN WSPLGYRPHP ERRGEFITNA LWQSAYTALA
651 GLHSLSSWDE EKGHAASLQG IGLLVHQKDK NGFKGFRSHM TGYSATTEAT
701 SSQSPNFSLG FAQEESKAKE HESQNSTSSH HYFSGMCIEN TLFKEWTRLS
751 VSLAYMFTSE HTHTMYQGLL EGNSQGSFHN HTLAGALSCV FLPQPHGESL
801 QIYPFITALA IRGNLAAFQE SGDHAREFSL HRPLTDVSLP VGIRASWKNH
851 HRVPLVWLTE ISYRSTLYRQ DPELHSKLLI SQGTWTTQAT PVTYNALGIK
901 VKNTMQVFPK VTLSLDYSAD ISSSTLSHYL NVASRMRF*
```

A predicted signal peptide is highlighted.

The cp6751 nucleotide sequence <SEQ ID 6> is:

```
      1 ATGCGCTTTT TTTGCTTCGG AATGTTGCTT CCTTTTACTT TTGTATTGGC
    51 TAATGAAGGT CTCCAACTTC CTTTGGAGAC CTATATTACA TTAAGTCCTG
  101 AATATCAAGC AGCCCCTCAA GTAGGGTTTA CTCATAACCA AAATCAAGAT
  151 CTCGCAATTG TCGGGAATCA CAATGATTTC ATCTTGGACT ATAAGTACTA
  201 TCGGTCGAAT GGAGGTGCTC TTACCTGTAA GAATCTTCTG ATCTCTGAAA
  251 ATATAGGGAA TGTCTTCTTT GAGAAGAATG TCTGTCCCAA TTCTGGCGGG
  301 GCAATTTATG CTGCTCAAAA TTGCACGATC TCCAAGAATC AGAACTATGC
  351 ATTTACTACA AACTTGGTCT CTGACAATCC TACAGCCACT GCGGGATCAC
  401 TATTGGGTGG AGCTCTCTTT GCCATAAATT GCTCTATTAC TAATAACCTA
  451 GGACAGGGAA CTTTCGTTGA CAATCTCGCT TTAAATAAGG GGGGTGCCCT
  501 CTATACTGAG ACGAACTTAT CTATTAAAGA CAATAAAGGC CCGATCATAA
  551 TCAAGCAGAA TCGGGCACTA AATTCGGACA GTTTAGGAGG AGGGATTTAT
  601 AGTGGGAACT CTCTAAATAT AGAGGGAAAT TCTGGAGCTA TACAGATCAC
  651 AAGCAACTCT TCAGGATCTG GGGGAGGCAT ATTTTCTACC CAAACACTCA
  701 CGATCTCCTC GAATAAAAAA CTCATAGAAA TCAGTGAAAA TTCCGCGTTC
  751 GCAAATAACT ATGGATCGAA CTTCAATCCA GGAGGAGGAG GTCTTACTAC
  801 CACCTTTTGC ACGATATTGA ACAACCGAGA AGGGGTACTC TTTAACAATA
  851 ACCAAAGCCA GAGCAACGGT GGAGCCATTC ATGCGAAATC TATCATTATC
  901 AAAGAAAATG GTCCTGTATA CTTTTTAAAT AACACTGCAA CTCGGGGAGG
  951 GGCTCTCCTC AACTTATCAG CAGGTTCTGG AAACGGAAGC TTCATCTTAT
1001 CTGCAGATAA TGGAGATATT ATCTTTAACA ATAATACGGC CTCCAAGCAT
1051 GCCCTCAATC CTCCATACAG AAACGCCATT CACTCGACTC CTAATATGAA
1101 TCTGCAAATA GGAGCCCGTC CCGGCTATCG AGTGCTGTTC TATGATCCCA
1151 TAGAACATGA GCTCCCTTCC TCCTTCCCCA TACTCTTTAA TTTCGAAACC
1201 GGTCATACAG GTACAGTTTT ATTTTCAGGG GAACATGTAC ACCAGAACTT
1251 TACCGATGAA ATGAATTTCT TTTCCTATTT AAGGAACACT TCGGAACTAC
1301 GTCAAGGAGT CCTTGCTGTT GAAGATGGTG CGGGGCTGGC CTGCTATAAG
1351 TTCTTCCAAC GAGGAGGCAC TCTACTTCTA GGTCAAGGTG CGGTGATCAC
1401 GACAGCAGGA ACGATTCCCA CACCATCCTC AACACCAACG ACAGTAGGAA
1451 GTACTATAAC TTTAAATCAC ATTGCCATTG ACCTTCCTTC TATTCTTTCT
1501 TTTCAAGCTC AGGCTCCAAA AATTTGGATT TACCCCACAA AAACAGGATC
1551 TACCTATACT GAAGATTCCA ACCCGACAAT CACAATCTCA GGAACTCTCA
1601 CCTTACGCAA CAGCAACAAC GAAGATCCCT ACGATAGTCT GGATCTCTCG
1651 CACTCTCTTG AGAAAGTTCC CCTTCTTTAT ATTGTCGATG TCGCTGCACA
1701 AAAAATTAAC TCTTCGCAAC TGGATCTATC CACATTAAAT TCTGGCGAAC
1751 ACTATGGGTA TCAAGGCATC TGGTCGACCT ATTGGGTAGA AACTACAACA
1801 ATCACGAACC CTACATCTCT ACTAGGCGCG AATACAAAAC ACAAGCTGCT
1851 CTATGCAAAC TGGTCTCCTC TAGGCTACCG TCCTCATCCC GAACGTCGAG
1901 GAGAATTCAT TACGAATGCC TTGTGGCAAT CGGCATATAC GGCTCTTGCA
1951 GGACTCCACT CCCTCTCCTC CTGGGATGAA GAGAAGGGTC ATGCAGCTTC
2001 CCTACAAGGC ATTGGTCTTC TGGTTCATCA AAAAGACAAA AACGGTTTTA
2051 AGGGATTTCG TAGTCATATG ACAGGTTATA GTGCTACCAC CGAAGCAACC
2101 TCTTCTCAAA GTCCGAATTT CTCTTTAGGA TTTGCTCAGT TCTTCTCCAA
2151 AGCTAAAGAA CATGAATCTC AAAATAGCAC GTCCTCTCAC CACTATTTCT
2201 CTGGAATGTG CATAGAAAAT ACTCTCTTCA AAGAGTGGAT ACGTCTATCT
2251 GTGTCTCTTG CTTATATGTT TACCTCGGAA CATACCCATA CAATGTATCA
2301 GGGTCTCCTG GAAGGGAACT CTCAGGGATC TTTCCACAAC CATACCTTAG
2351 CAGGGGCTCT CTCCTGTGTT TTCTTACCTC AACCTCACGG CGAGTCCCTG
2401 CAGATCTATC CCTTTATTAC TGCCTTAGCC ATCCGAGGAA ATCTTGCTGC
2451 GTTTCAAGAA TCTGGAGACC ATGCTCGGGA ATTTTCCCTA CACCGCCCCC
2501 TAACGGACGT CTCCCTCCCT GTAGGAATCC GCGCTTCTTG GAAGAACCAC
2551 CACCGAGTTC CCCTAGTCTG GCTCACAGAA ATTTCCTATC GCTCTACTCT
2601 CTATAGGCAA GATCCTGAAC TCCACTCGAA ATTACTGATT AGCCAAGGTA
2651 CGTGGACGAC GCAGGCCACT CCTGTGACCT ACAATGCTTT AGGGATCAAA
2701 GTGAAAAATA CCATGCAGGT GTTTCCTAAA GTCACTCTCT CCTTAGATTA
2751 CTCTGCGGAT ATTTCTTCCT CCACGCTGAG TCACTACTTA AACGTGGCGA
2801 GTAGAATGAG ATTTTAA
```

The PSORT algorithm predicts an outer membrane location (0.923).

The protein was expressed in E. coli and purified as a GST-fusion product, as shown in FIG. 3A, and also in his-tagged form. The GST-fusion recombinant protein was used to immunize mice, whose sera were used in a Western blot (FIG. 3B) and for FACS analysis (FIG. 3C).

This protein also showed good cross-reactivity with human sera, including sera from patients with pneumonitis.

These experiments show that cp6751 is a surface-exposed and immunoaccessible protein, and that it is a useful immunogen. These properties are not evident from the sequence alone.

### Example 4

The following C. pneumoniae protein (PID 4376752) was expressed <SEQ ID 7; cp6752>:

```
    1 MFGMTPAVYS LQTDSLEKFA LERDEEFRTS FPLLDSLSTL TGFSPITTFV
  51 GNRHNSSQDI VLSNYKSIDN ILLLWTSAGG AVSCNNFLLS NVEDHAFFSK
101 NLAIGTGGAI ACQGACTITK NRGPLIFFSN RGLNNASTGG ETRGGAIACN
151 GDFTISQNQG TFYFVNNSVN NWGGALSTNG HCRIQSNRAP LLFFNNTAPS
201 GGGALRSENT TISDNTRPIY FKNNCGNNGG AIQTSVTVAI KNNSGSVIFN
251 NNTALSGSIN SGNGSGGAIY TTNLSIDDNP GTILFNNNYC IRDGGAICTQ
301 FLTIKNSGHV YFTNNQGNWG GALMLLQDST CLLFAEQGNI AFQNNEVFLT
351 TFGRYNAIHC TPNSNLQLGA NKGYTTAFFD PIEHQHPTTN PLIFNPNANH
401 QGTILFSSAY TPEASDYENN FISSSKNTSE LRNGVLSIED RAGWQFYKFT
451 QKGGILKLGH AASTATTANS ETPSTSVGSQ VIINNLAINL PSILAKGKAP
501 TLWIRPLQSS APFTEDNNPT ITLSGPLTLL NEENRDPYDS IDLSEPLQNI
551 HLLSLSDVTA RHINTDNFHP ESLNATEHYG YQGIWSPYWV ETITTTNNAS
601 IETANTLYRA LYANWTPLGY KVNPEYQGDL ATTPLWQSFH TMFSLLRSYN
651 RTGDSDIERP FLEIQGIADG LFVHQNSIPG APGFRIQSTG YSLQASSETS
701 LHQKISLGFA QFFTRTKEIG SSNNVSAHNT VSSLYVELPW FQEAFATSTV
751 LAYGYGDHHL HSLHPSHQEQ AEGTCYSHTL AAAIGCSFPW QQKSYLHLSP
801 FVQAIAIRSH QTAFEEIGDN PRKFVSQKPF YNLTLPLGIQ GKWQSKFHVP
851 TEWTLELSYQ PVLYQQNPQI GVTLLASGGS WDILGHNYVR NALGYKVHNQ
901 TALFRSLDLF LDYQGSVSSS TSTHHLQAGS TLKF*
```

The cp6752 nucleotide sequence <SEQ ID 8> is:

```
      1 ATGTTCGGGA TGACTCCTGC AGTGTATAGT TTACAAACGG ACTCCCTTGA
    51 AAAGTTTGCT TTAGAGAGGG ATGAAGAGTT TCGTACGAGC TTTCCTCTCT
  101 TAGACTCTCT CTCCACTCTT ACAGGATTTT CTCCAATAAC TACGTTTGTT
  151 GGAAATAGAC ATAATTCCTC TCAAGACATT GTACTTTCTA ACTACAAGTC
  201 TATTGATAAC ATCCTTCTTC TTTGGACATC GGCTGGGGGA GCTGTGTCCT
  251 GTAATAATTT CTTATTATCA AATGTTGAAG ACCATGCCTT CTTCAGTAAA
  301 AATCTCGCGA TTGGGACTGG AGGCGCGATT GCTTGCCAGG GAGCCTGCAC
  351 AATCACGAAG AATAGAGGAC CCCTTATTTT TTTCAGCAAT CGAGGTCTTA
  401 ACAATGCGAG TACAGGAGGA GAAACTCGTG GGGGTGCGAT TGCCTGTAAT
  451 GGAGACTTCA CGATTTCTCA AAATCAAGGG ACTTTCTACT TTGTCAACAA
  501 TTCCGTCAAC AACTGGGGAG GAGCCCTCTC CACCAATGGA CACTGCCGCA
  551 TCCAAAGCAA CAGGGCACCT CTACTCTTTT TTAACAATAC AGCCCCTAGT
  601 GGAGGGGGTG CGCTTCGTAG TGAAAATACA ACGATCTCTG ATAACACGCG
  651 TCCTATTTAT TTTAAGAACA ACTGTGGGAA CAATGGCGGG GCCATTCAAA
  701 CAAGCGTTAC TGTTGCGATA AAAAATAACT CCGGGTCGGT GATTTTCAAT
  751 AACAACACAG CGTTATCTGG TTCGATAAAT TCAGGAAATG GTTCAGGAGG
  801 GGCGATTTAT ACAACAAACC TATCCATAGA CGATAACCCT GGAACTATTC
  851 TTTTCAATAA TAACTACTGC ATTCGCGATG GCGGAGCTAT CTGTACACAA
  901 TTTTTGACAA TCAAAAATAG TGGCCACGTA TATTTCACCA ACAATCAAGG
  951 AAACTGGGGA GGTGCTCTTA TGCTCCTACA GGACAGCACC TGCCTACTCT
1001 TCGCGGAACA AGGAAATATC GCATTTCAAA ATAATGAGGT TTTCCTCACC
1051 ACATTTGGTA GATACAACGC CATACATTGT ACACCAAATA GCAACTTACA
1101 ACTTGGAGCT AATAAGGGGT ATACGACTGC TTTTTTTGAT CCTATAGAAC
1151 ACCAACATCC AACTACAAAT CCTCTAATCT TTAATCCCAA TGCGAACCAT
1201 CAGGGAACGA TCTTATTTTC TTCAGCCTAT ATCCCAGAAG CTTCTGACTA
1251 CGAAAATAAT TTCATTAGCA GCTCGAAAAA TACCTCTGAA CTTCGCAATG
1301 GTGTCCTCTC TATCGAGGAT CGTGCGGGAT GGCAATTCTA TAAGTTCACT
1351 CAAAAAGGAG GTATCCTTAA ATTAGGGCAT GCGGCGAGTA TTGCAACAAC
1401 TGCCAACTCT GAGACTCCAT CAACTAGTGT AGGCTCCCAG GTCATCATTA
1451 ATAACCTTGC GATTAACCTC CCCTCGATCT TAGCAAAAGG AAAAGCTCCT
1501 ACCTTGTGGA TCCGTCCTCT ACAATCTAGT GCTCCTTTCA CAGAGGACAA
1551 TAACCCTACA ATTACTTTAT CAGGTCCTCT GACACTCTTA AATGAGGAAA
1601 ACCGCGATCC CTACGACAGT ATAGATCTCT CTGAGCCTTT ACAAAACATT
1651 CATCTTCTTT CTTTATCGGA TGTAACAGCA CGTCATATCA ATACCGATAA
1701 CTTTCATCCT GAAAGCTTAA ATGCGACTGA GCATTACGGT TATCAAGGCA
1751 TCTGGTCTCC TTATTGGGTA GAGACGATAA CAACAACAAA TAACGCTTCT
1801 ATAGAGACGG CAAACACCCT CTACAGAGCT CTGTATGCCA ATTGGACTCC
1851 CTTAGGATAT AAGGTCAATC CTGAATACCA AGGAGATCTT GCTACGACTC
1901 CCCTATGGCA ATCCTTTCAT ACTATGTTCT CTCTATTAAG AAGTTATAAT
1951 CGAACTGGTG ATTCTGATAT CGAGAGGCCT TTCTTAGAAA TTCAAGGGAT
2001 TGCCGACGGC CTCTTTGTTC ATCAAAATAG CATCCCCGGG GCTCCAGGAT
2051 TCCGTATCCA ATCTACAGGG TATTCCTTAC AAGCATCCTC CGAAACTTCT
2101 TTACATCAGA AAATCTCCTT AGGTTTTGCA CAGTTCTTCA CCCGCACTAA
2151 AGAAATCGGA TCAAGCAACA ACGTCTCGGC TCACAATACA GTCTCTTCAC
2201 TTTATGTTGA GCTTCCGTGG TTCCAAGAGG CCTTTGCAAC ATCCACAGTG
2251 TTAGCGTATG GCTATGGGGA CCATCACCTC CACAGCCTAC ATCCCTCACA
2301 TCAAGAACAG GCAGAAGGGA CGTGTTATAG CCATACATTA GCAGCAGCTA
2351 TCGGCTGTTC TTTCCCTTGG CAACAGAAAT CCTATCTTCA CCTCAGCCCG
2401 TTCGTTCAGG CAATTGCAAT ACGTTCTCAC CAAACAGCGT TCGAAGAGAT
2451 TGGTGACAAT CCCCGAAAGT TTGTCTCTCA AAAGCCTTTC TATAATCTGA
2501 CCTTACCTCT AGGAATCCAA GGAAAATGGC AGTCAAAATT CCACGTACCT
2551 ACAGAATGGA CTCTAGAACT TTCTTACCAA CCGGTACTCT ATCAACAAAA
2601 TCCCCAAATC GGTGTCACGC TACTTGCGAG CGGAGGTTCC TGGGATATCC
2651 TAGGCCATAA CTATGTTCGC AATGCTTTAG GGTACAAAGT CCACAATCAA
2701 ACTGCGCTCT TCCGTTCTCT CGATCTATTC TTGGATTACC AAGGATCGGT
2751 CTCCTCCTCG ACATCTACGC ACCATCTCCA AGCAGGAAGT ACCTTAAAAT
2801 TCTAA
```

The PSORT algorithm predicts a cytoplasmic location (0.138).

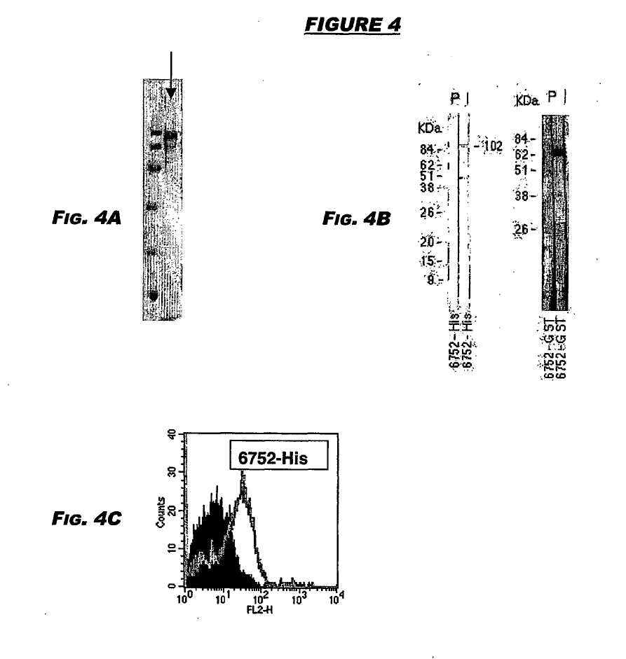

The protein was expressed in E. coli and purified as a his-tag product, as shown in FIG. 4A, and also as a GST-fusion. The recombinant proteins were used to immunize mice, whose sera were used in a Western blot (4B) and the his-tagged protein was used for FACS analysis (4C).

The cp6752 protein was also identified in the 2D-PAGE experiment (Cpn0467).

These experiments show that cp6752 is a surface-exposed and immunoaccessible protein, and that it is a useful immunogen. These properties are not evident from the sequence alone.

### Example 5

The following C. pneumoniae protein (PID 4376850) was expressed <SEQ ID 9; cp6850>:

1 MKKAVLIAAM FCGVVSLSSC CRTVDCCFED PCAPSSCNPC EVIRKKERSC
51 GGNACGSYVP SCSNPCGSTE CNSQSPQVKG CTSPDGRCKQ *

A predicted signal peptide is highlighted.

The cp6850 nucleotide sequence <SEQ ID 10> is:

```
    1 ATGAAGAAAG CTGTTTTAAT TGCTGCAATG TTTTGTGGAG TAGTTAGCTT
  51 AAGTAGCTGC TGCCGCATTG TAGATTGTTG TTTTGAGGAT CCTTGCGCAC
101 CCTCTTCTTG CAATCCTTGT GAAGTAATAA GAAAAAAAGA AAGATCTTGC
151 GGCGGTAATG CTTGTGGGTC CTACGTTCCT TCTTGTTCTA ATCCATGTGG
201 TTCAACAGAG TGTAACTCTC AAAGCCCACA AGTTAAAGGT TGTACATCAC
251 CTGATGGCAG ATGCAAACAG TAA
```

The PSORT algorithm predicts an inner membrane location (0.329).

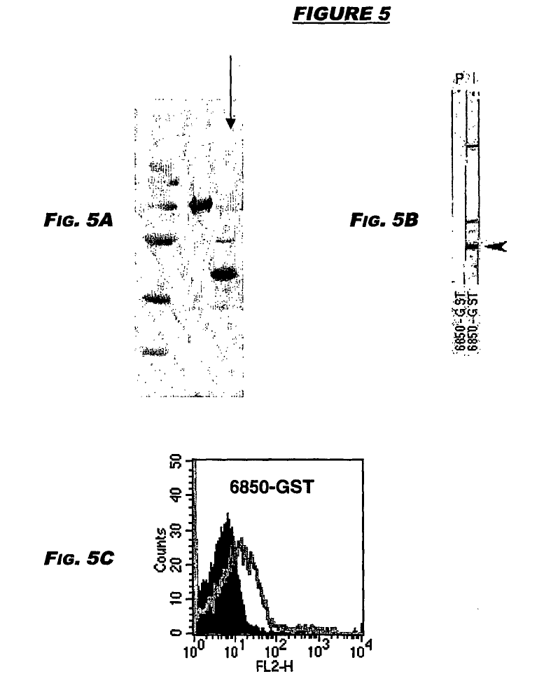

The protein was expressed in E. coli and purified as a GST-fusion product, as shown in FIG. 5A. The recombinant protein was used to immunize mice, whose sera were used in a Western blot (FIG. 5B) and for FACS analysis (FIG. 5B). A his-tagged protein was also expressed.

These experiments show that cp6850 is a surface-exposed and immunoaccessible protein, and that it is a useful immunogen. These properties are not evident from the sequence alone.

### Example 6

The following C. pneumoniae protein (PID 4376900) was expressed <SEQ ID 11; cp6900>:

```
    1 MKIKFSWKVN FLICLLAVGL IFFGCSRVKR EVLVGRDATW FPKQFGIYTS
  51 DTNAFLNDLV SEINYKENLN INIVNQDWVH LFENLDDKKT QGAFTSVLPT
101 LEMLEHYQES DPILLTGPVL VVAQDSPYQS IEDLKGRLIG VYKFDSSVLV
151 AQNIPDAVIS LYQHVPIALE ALTSNCYDAL LAPVIEVTAL IETAYKGRLK
201 IISKPLNADG LRLAILKGTN GDLLEGFNAG LVKTRRSGKY DAIKQRYRLP
```

The cp6900 nucleotide sequence <SEQ ID 12> is:

```
    1 GTGAAGATAA AATTTTCTTG GAAGGTAAAT TTTTTAATAT GTTTACTGGC
  51 TGTGGGACTG ATCTTTTTCG GGTGCTCTCG AGTAAAAAGA GAAGTTCTCG
101 TAGGTCGTGA TGCCACCTGG TTTCCAAAAC AATTCGGCAT TTATACATCC
151 GATACCAACG CATTTTTAAA CGATCTTGTT TCTGAGATTA ACTATAAAGA
201 GAATCTAAAT ATTAATATTG TAAATCAAGA TTGGGTGCAT CTCTTTGAGA
251 ATTTAGATGA TAAAAAGACC CAAGGAGCAT TTACATCTGT ATTGCCTACT
301 CTTGAGATGC TCGAACACTA TCAATTTTCT GATCCCATTT TACTCACAGG
351 TCCTGTCCTT GTCGTCGCTC AAGACTCTCC TTACCAATCT ATAGAGGATC
401 TTAAAGGTCG TCTTATTGGA GTGTATAAGT TTGACTCTTC AGTTCTTGTA
451 GCTCAAAATA TCCCTGACGC TGTGATTAGC CTCTACCAAC ATGTTCCAAT
501 AGCATTGGAA GCCTTAACAT CGAATTGTTA CGACGCTCTT CTAGCTCCTG
551 TAATTGAAGT GACCGCGCTA ATAGAAACAG CATATAAAGG AAGACTGAAA
601 ATTATTTCAA AACCCTTAAA CGCAGATGGT TTGCGGCTTG CAATACTGAA
651 AGGGACAAAC GGAGATTTGC TTGAAGGGTT TAACGCAGGA CTTGTGAAAA
701 CACGACGCTC AGGAAAATAC GATGCTATAA AACAGCGGTA TCGTCTTCCC
751 TAA
```

The PSORT algorithm predicts an inner membrane location (0.452).

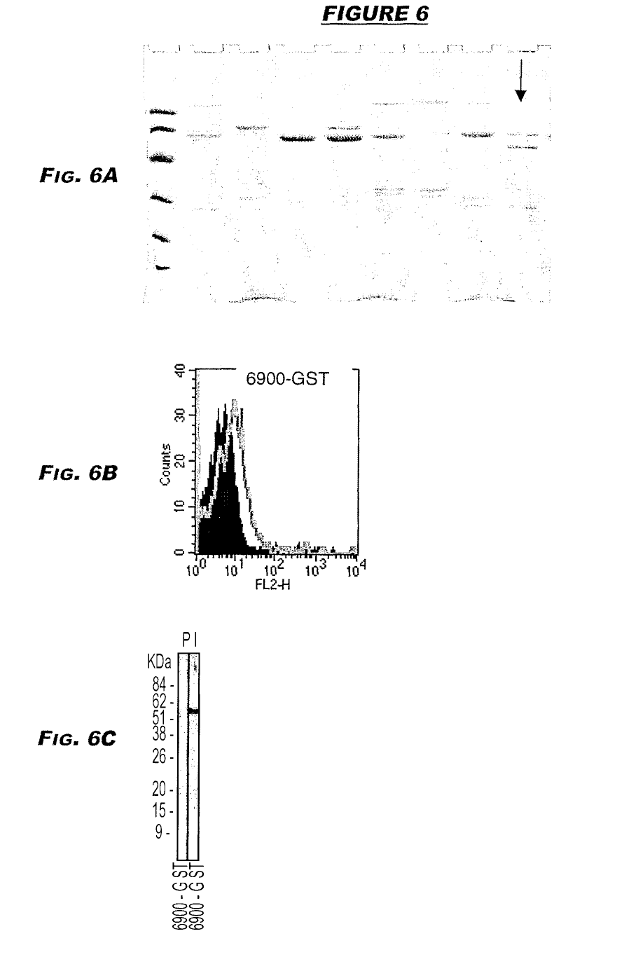

The protein was expressed in E. coli and purified as a GST-fusion product, as shown in FIG. 6A. The recombinant protein was used to immunize mice, whose sera were used for FACS analysis (FIG. 6B). A his-tagged protein was also expressed.

The cp6900 protein was also identified in the 2D-PAGE experiment (Cpn0604).

These experiments show that cp6900 is a surface-exposed and immunoaccessible protein, and that it is a useful immunogen. These properties are not evident from the sequence alone.

### Example 7

The following C. pneumoniae protein (PID 4377033) was expressed <SEQ ID 13; cp7033>:

```
    1 MVNPIGPGPI DETERTPPAD LSAQGLEASA ANKSAEAQRI AGAEAKPKES
  51 KTDSVERWSI LRSAVNALMS LADKLGTASS NSSSSTSRSA DVDSTTATAP
101 TPPPPTFDDY KTQAQTAYDT TFTSTSLADT QAALVSLQDA VTNIKDTAAT
151 DEETAIAAEW ETKNADAVKV GAQITELAKY ASDNQAILDS LGKLTSFDLL
201 QAALLQSVAN NNKAAELLKE MQDNPVVPGK TRAIAQSLVD QTDATATQIE
251 KDGNAIRDAY FAGQNASGAV ENAKSNNSIS NTDSAKAAIA TAKTQIAEAQ
301 KKFPDSPTLQ EAEQMVIQAE KDLKNIKPAD GSDVPNPGTT VGGSKQQGSS
351 IGSIRVSMLL DDAENETASI LMSGFRQMIH MFNTENPDSQ AAQQELAAQA
401 RAAKAAGDDS AAAALADAQK ALEAALGKAG QQQGILNALG QIASAAVVSA
451 GVPPAAASSI GSSVKQLYKT SKSTGSDYKT QISAGYDAYK SINDAYGPAR
501 NDATRDVINN VSTPALTRSV PPARTEARGP EKTDQALARV ISGNSRTLGD
551 VYSQVSALQS VMQIIQSNPQ ANNEEIRQKL TSAVTKPPQF GYPYVQLSND
601 STQKFIAKLE SLFAEGSRTA AEIKALSEET NSLFIQQVLV NIGSLYSGYL
651 Q*
```

The cp7033 nucleotide sequence <SEQ ID 14> is:

```
      1 ATGGTTAATC CTATTGGTCC AGGTCCTATA GACGAAACAG AACGCACACC
    51 TCCCGCAGAT CTTTCTGCTC AAGGATTGGA GGCGAGTGCA GCAAATAAGA
  101 GTGCGGAAGC TCAAAGAATA GCAGGTGCGG AAGCTAAGCC TAAAGAATCT
  151 AAGACCGATT CTGTAGAGCG ATGGAGCATC TTGCGTTCTG CAGTGAATGC
  201 TCTCATGAGT CTGGCAGATA AGCTGGGTAT TGCTTCTAGT AACAGCTCGT
  251 CTTCTACTAG CAGATCTGCA GACGTGGACT CAACGACAGC GACCGCACCT
  301 ACGCCTCCTC CACCCACGTT TGATGATTAT AAGACTCAAG CGCAAACAGC
  351 TTACGATACT ATCTTTACCT CAACATCACT AGCTGACATA CAGGCTGCTT
  401 TGGTGAGCCT CCAGGATGCT GTCACTAATA TAAAGGATAC AGCGGCTACT
  451 GATGAGGAAA CCGCAATCGC TGCGGAGTGG GAAACTAAGA ATGCCGATGC
  501 AGTTAAAGTT GGCGCGCAAA TTACAGAATT AGCGAAATAT GCTTCGGATA
  551 ACCAAGCGAT TCTTGACTCT TTAGGTAAAC TGACTTCCTT CGACCTCTTA
  601 CAGGCTGCTC TTCTCCAATC TGTAGCAAAC AATAACAAAG CAGCTGAGCT
  651 TCTTAAAGAG ATGCAAGATA ACCCAGTAGT CCCAGGGAAA ACGCCTGCAA
  701 TTGCTCAATC TTTAGTTGAT CAGACAGATG CTACAGCGAC ACAGATAGAG
  751 AAAGATGGAA ATGCGATTAG GGATGCATAT TTTGCAGGAC AGAACGCTAG
  801 TGGAGCTGTA GAAAATGCTA AATCTAATAA CAGTATAAGC AACATAGATT
  851 CAGCTAAAGC AGCAATCGCT ACTGCTAAGA CACAAATAGC TGAAGCTCAG
  901 AAAAAGTTCC CCGACTCTCC AATTCTTCAA GAAGCGGAAC AAATGGTAAT
  951 ACAGGCTGAG AAAGATCTTA AAAATATCAA ACCTGCAGAT GGTTCTGATG
1001 TTCCAAATCC AGGAACTACA GTTGGAGGCT CCAAGCAACA AGGAAGTAGT
1051 ATTGGTAGTA TTCGTGTTTC CATGCTGTTA GATGATGCTG AAAATGAGAC
1101 CGCTTCCATT TTGATGTCTG GGTTTCGTCA GATGATTCAC ATGTTCAATA
1151 CGGAAAATCC TGATTCTCAA GCTGCCCAAC AGGAGCTCGC AGCACAAGCT
1201 AGAGCAGCGA AAGCCGCTGG AGATGACAGT GCTGCTGCAG CGCTGGCAGA
1251 TGCTCAGAAA GCTTTAGAAG CGGCTCTAGG TAAAGCTGGG CAACAACAGG
1301 GCATACTCAA TGCTTTAGGA CAGATCGCTT CTGCTGCTGT TGTGAGCGCA
1351 GGAGTTCCTC CCGCTGCAGC AAGTTCTATA GGGTCATCTG TAAAACAGCT
1401 TTACAAGACC TCAAAATCTA CAGGTTCTGA TTATAAAACA CAGATATCAG
1451 CAGGTTATGA TGCTTACAAA TCCATCAATG ATGCCTATGG TAGGGCACGA
1501 AATGATGCGA CTCGTGATGT GATAAACAAT GTAAGTACCC CCGCTCTCAC
1551 ACGATCCGTT CCTAGAGCAC GAACAGAAGC TCGAGGACCA GAAAAAACAG
1601 ATCAAGCCCT CGCTAGGGTG ATTTCTGGCA ATAGCAGAAC TCTTGGAGAT
1651 GTCTATAGTC AAGTTTCGGC ACTACAATCT GTAATGCAGA TCATCCAGTC
1701 GAATCCTCAA GCGAATAATG AGGAGATCAG ACAAAAGCTT ACATCGGCAG
1751 TGACAAAGCC TCCACAGTTT GGCTATCCTT ATGTGCAACT TTCTAATGAC
1801 TCTACACAGA AGTTCATAGC TAAATTAGAA AGTTTGTTTG CTGAAGGATC
1851 TAGGACAGCA GCTGAAATAA AAGCACTTTC CTTTGAAACG AACTCCTTGT
1901 TTATTCAGCA GGTGCTGGTC AATATCGGCT CTCTATATTC TGGTTATCTC
1951 CAATAA
```

The PSORT algorithm predicts a cytoplasmic location (0.272).

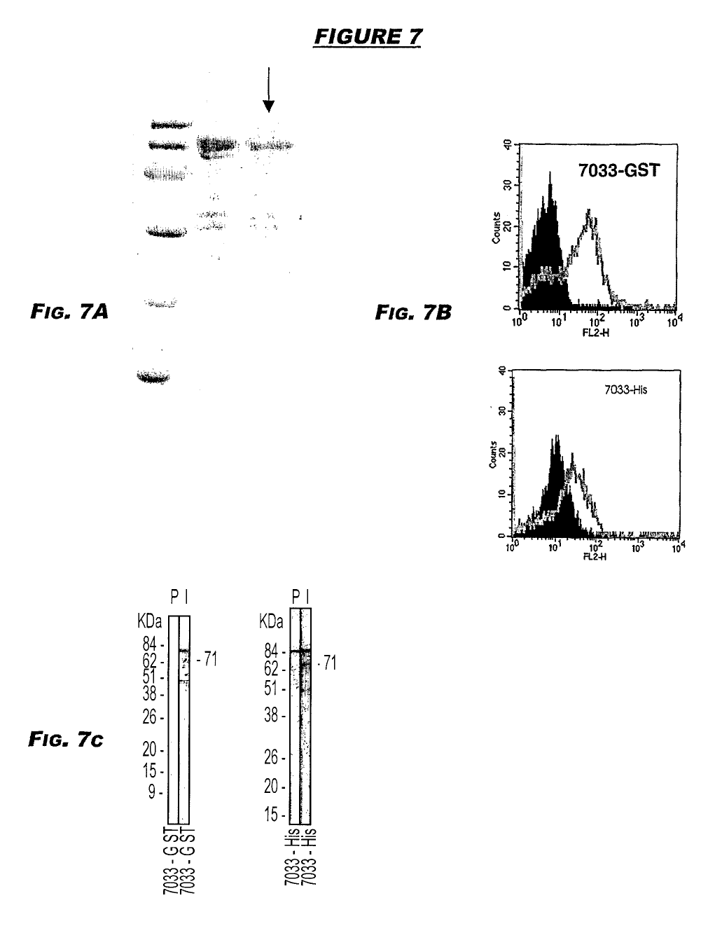

The protein was expressed in E. coli and purified as a GST-fusion product, as shown in FIG. 7A. A his-tagged protein was also expressed. The recombinant proteins were used to immunize mice, whose sera were used for FACS (FIG. 7B) and Western blot (7C) analyses.

The cp7033 protein was also identified in the 2D-PAGE experiment (Cpn0728) and showed good cross-reactivity with human sera, including sera from patients with pneumonitis.

These experiments show that cp7033 a surface-exposed and immunoaccessible protein, and that it is a useful immunogen. These properties are not evident from the sequence alone.

### Example 8

The following C. pneumoniae protein (PID 6172321) was expressed <SEQ ID 15; cp0017>:

```
    1 MGIKGTGIIV WVDDATAKTK NATLTWTKTG YKPNPERQGP LVPNSLWGSF
  51 VDVRSIQSLM DRSTSSLSSS TNLWVSGIAD FLHEDQKGNQ RSYRHSSAGY
101 ALGGGFFTAS ENFFNFAFCQ LFGYDKDHLV AKNHTHVYAG AMSYRHLGES
151 KTLAKILSGN SDSLPFVFNA RFAYGHTDNN MTTKYTGYSP VKGSWGNDAF
201 GIECGGAIPV VASGRRSWVD THTPFLNLEM IYAHQNDFKE NGTEGRSFQS
251 EDLFNLAVPV GIKFEKFSDK STYDLSIAYV PDVIRNDPGC TTTLMVSGDS
301 WSTCGTSLSR QALLVRAGNH HAFASNEEVE SQFEVELRGS SRSYAIDLGG
351 RFGF*
```

The cp0017 nucleotide sequence <SEQ ID 16> is:

```
      1 ATGGGTATCA AGGGAACTGG AATAATTGTT TGGGTCGACG ATGCAACTGC
    51 AAAAACAAAA AATGCTACCT TAACTTGGAC TAAAACAGGA TACAAGCCGA
  101 ATCCAGAACG TCAGGGACCT TTGGTTCCTA ATAGCCTGTG GGGTTCTTTT
  151 GTCGATGTCC GCTCCATTCA GAGCCTCATG GACCGGAGCA CAAGTTCGTT
  201 ATCTTCGTCA ACAAATTTGT GGGTATCAGG AATCGCGGAC TTTTTGCATG
  251 AAGATCAGAA AGGAAACCAA CGTAGTTATC GTCATTCTAG CGCGGGTTAT
  301 GCATTAGGAG GAGGATTCTT CACGGCTTCT GAAAATTTCT TTAATTTTGC
  351 TTTTTGTCAG CTTTTTGGCT ACGACAAGGA CCATCTTGTG GCTAAGAACC
  401 ATACCCATGT ATATGCAGGG GCAATGAGTT ACCGACACCT CGGAGAGTCT
  451 AAGACCCTCG CTAAGATTTT GTCAGGAAAT TCTGACTCCC TACCTTTTGT
  501 CTTCAATGCT CGGTTTGCTT ATGGCCATAC CGACAATAAC ATGACCACAA
  551 AGTACACTGG CTATTCTCCT GTTAAGGGAA GCTGGGGAAA TGATGCCTTC
  601 GGTATAGAAT GTGGAGGAGC TATCCCGGTA GTTGCTTCAG GACGTCGGTC
  651 TTGGGTGGAT ACCCACACGC CATTTCTAAA CCTAGAGATG ATCTATGCAC
  701 ATCAGAATGA CTTTAAGGAA AACGGCACAG AAGGCCGTTC TTTCCAAAGT
  751 GAAGACCTCT TCAATCTAGC GGTTCCTGTA GGGATAAAAT TTGAGAAATT
  801 CTCCGATAAG TCTACGTATG ATCTCTCCAT AGCTTACGTT CCCGATGTGA
  851 TTCGTAATGA TCCAGGCTGC ACGACAACTC TTATGGTTTC TGGGGATTCT
  901 TGGTCGACAT GTGGTACAAG CTTGTCTAGA CAAGCTCTTC TTGTACGTGC
  951 TGGAAATCAT CATGCCTTTG CTTCAAACTT TGAAGTTTTC AGTCAGTTTG
1001 AAGTCGAGTT GCGAGGTTCT TCTCGTAGCT ATGCTATCGA TCTTGGAGGA
1051 AGATTCGGAT TTTAA
```

This sequence is frame-shifted with respect to cp0016.

The PSORT algorithm predicts a cytoplasmic location (0.075).

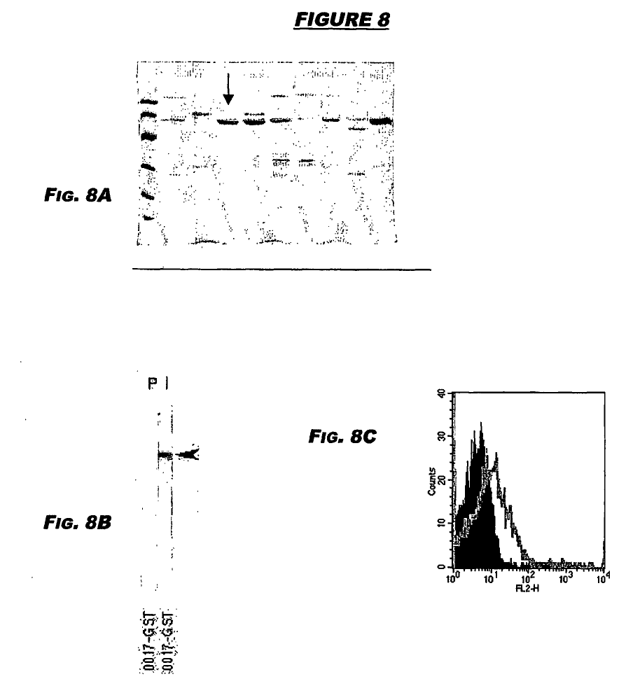

The protein was expressed in E. coli and purified as a GST-fusion product, as shown in FIG. 8A. The recombinant protein was used to immunize mice, whose sera were used in a Western blot (FIG. 8B) and for FACS analysis (FIG. 8C). A his-tagged protein was also expressed.

This protein also showed good cross-reactivity with human sera, including sera from patients with pneumonitis.

These experiments show that cp0017 is a surface-exposed and immunoaccessible protein, and that it is a useful immunogen. These properties are not evident from the sequence alone.

### Example 9

The following C. pneumoniae protein (PID 6172315) was expressed <SEQ ID 17; cp0014>:

```
    1 MKSSFPKFVF STFAIFPLSM IATETVLDSS ASFDGNKNGN FSVRESQEDA
  51 GTTYLFKGNV TLENIPGTGT AITKSCFNNT KGDLTFTGNG NSLLFQTVDA
101 GTVAGAAVNS SVVDKSTTFI GFSSLSFIAS PGSSITTGKG AVSCSTGSLS
151 LTKMSVCSSA KTFQRIMAVL SPQKLFH*
```

The cp0014 nucleotide sequence <SEQ ID 18> is:

```
    1 ATGAAGTCTT CTTTCCCCAA GTTTGTATTT TCTACATTTG CTATTTTCCC
  51 TTTGTCTATG ATTGCTACCG AGACAGTTTT GGATTCAAGT GCGAGTTTCG
101 ATGGGAATAA AAATGGTAAT TTTTCAGTTC GTGAGAGTCA GGAAGATGCT
151 GGAACTACCT ACCTATTTAA GGGAAATGTC ACTCTAGAAA ATATTCCTGG
201 AACAGGCACA GCAATCACAA AAAGCTGTTT TAACAACACT AAGGGCGATT
251 TGACTTTCAC AGGTAACGGG AACTCTCTAT TGTTCCAAAC GGTGGATGCA
301 GGGACTGTAG CAGGGGCTGC TGTTAACAGC AGCGTGGTAG ATAAATCTAC
351 CACGTTTATA GGGTTTTCTT CGCTATCTTT TATTGCGTCT CCTGGAAGTT
401 CGATAACTAC CGGCAAAGGA GCCGTTAGCT GCTCTACGGG TAGCTTGAGT
451 TTGACAAAAA TGTCAGTTTG CTCTTCAGCA AAAACTTTTC AACGGATAAT
501 GGCGGTGCTA TCACCGCAAA AACTCTTTCA TTAA
```

This protein is frame-shifted with respect to cp0015.

The PSORT algorithm predicts an inner membrane location (0.047).

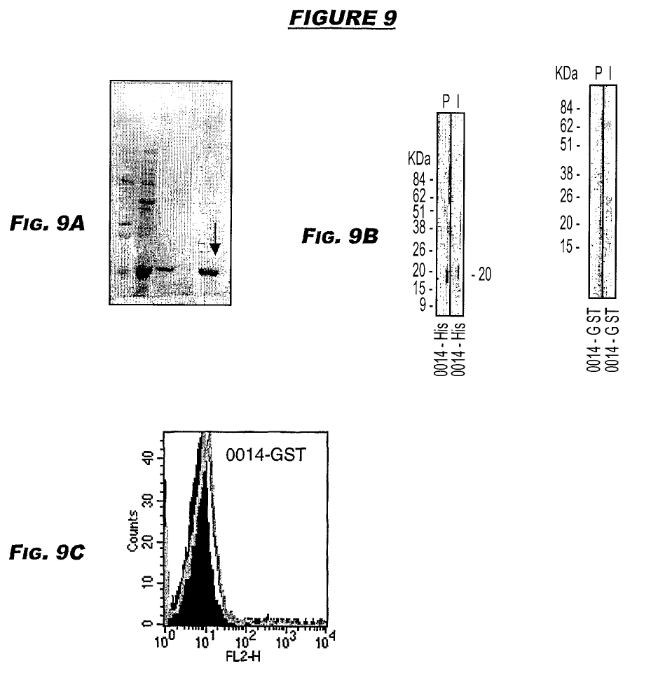

The protein was expressed in E. coli and purified as a his-tag product, as shown in FIG. 9A. A GST-fusion was also expressed. The recombinant proteins were used to immunize mice, whose sera were used in an immunoassay (FIG. 9B) and for FACS analysis (FIG. 9C).

This protein also showed good cross-reactivity with human sera, including sera from patients with pneumonitis.

These experiments suggest that cp0014 is a useful immunogen. These properties are not evident from the sequence alone.

### Example 10

The following C. pneumoniae protein (PID 6172317) was expressed <SEQ ID 19; cp0015>:

| | |
|---|---|
| 1 | MSALFSENTS SKKGGAIQTS DALTITGNQG EVSFSDNTSS DSGAAIFTEA |
| 51 | SVTISNNAKV SFIDNKVTGA SSSTTGDMSG GAICAYKTST DTKVTLTGNQ |
| 101 | MLLFSNNTST TAGGAIYVKK LELASGGLTL FSRNSVNGGT APKGGAIAIE |
| 151 | DSGELSLSAD SGDIVFLGNT VTSTTPGTNR SSIDLGTSAK MTALRSAAGR |
| 201 | AIYFYDPITT GSSTTVTDVL KVNETPADSA LQYTGNIIFT GEKLSETEAA |
| 251 | DSKNLTSKLL QPVTLSGGTL SLKHGVTLQT QAFTQQADSR LEMDVGTTLE |
| 301 | PADTSTINNL VINISSIDGA KKAKIETKAT SKNLTLSGTI TLLDPTGTFY |
| 351 | ENHSLRNPQS YDILELKASG TVTSTAVTPD PIMGEKFHYG YQGTWGPIVW |
| 401 | GTGASTTATF NWTKTGYIPN PERIGSLVPN SLWNAFIDIS SLHYLMETAN |
| 451 | EGLQGDRAFW CAGLSNFFHK DSTKTRRGFR HLSGGYVIGG NLHTCSDKIL |
| 501 | SAAFCQLFGR DRDYFVAKNQ GTVYGGTLYY QHNETYISLP CKLRPCSLSY |
| 551 | VPTEIPVLFS GNLSYTHTDN DLKTKYTTYP TVKGSWGNDS FALEFGGRAP |
| 601 | ICLDESALFE QYMPFMKLQF VYAHQEGFKE QGTEAREFGS SRLVNLALPI |
| 651 | GIRFDKESDC QDATYNLTLG YTVDLVRSNP DCTTTLRISG DSWKTFGTNL |
| 701 | ARQALVLRAG NHFCFNSNFE AFSQFSFELR GSSRNYNVDL GAKYQF* |

This sequence is frame-shifted with respect to cp0014.

The cp0015 nucleotide sequence <SEQ ID 20> is:

| | |
|---|---|
| 1 | ATGTCAGCTC TGTTTTCTGA AAATACCTCC TCAAAGAAAG GCGGAGCCAT |
| 51 | TCAGACTTCC GATGCCCTTA CCATTACTGG AAACCAAGGG GAAGTCTCTT |
| 101 | TTTCTGACAA TACTTCTTCG GATTCTGGAG CTGCAATTTT TACAGAAGCC |
| 151 | TCGGTGACTA TTTCTAATAA TGCTAAAGTT TCCTTTATTG ACAATAAGGT |
| 201 | CACAGGAGCG AGCTCCTCAA CAACGGGGGA TATGTCAGGA GGTGCTATCT |
| 251 | GTGCTTATAA AACTAGTACA GATACTAAGG TCACCCTCAC TGGAAATCAG |
| 301 | ATGTTACTCT TCAGCAACAA TACATCGACA ACAGCGGGAG GAGCTATCTA |
| 351 | TGTGAAAAAG CTCGAACTGG CTTCCGGAGG ACTTACCCTA TTCAGTAGAA |
| 401 | ATAGTGTCAA TGGAGGTACA GCTCCTAAAG GTGGAGCCAT AGCTATCGAA |
| 451 | GATAGTGGGG AATTGAGTTT ATCCGCCGAT AGTGGTGACA TTGTCTTTTT |
| 501 | AGGGAATACA GTCACTTCTA CTACTCCTGG GACGAATAGA AGTAGTATCG |
| 551 | ACTTAGGAAC GAGTGCAAAG ATGACAGCTT TGCGTTCTGC TGCTGGTAGA |
| 601 | GCCATCTACT TCTATGATCC CATAACTACA GGATCATCCA CAACAGTTAC |
| 651 | AGATGTCTTA AAAGTTAATG AGACTCCGGC AGATTCTGCA CTACAATATA |
| 701 | CAGGGAACAT CATCTTCACA GGAGAAAAGT TATCAGAGAC AGAGGCCGCA |
| 751 | GATTCTAAAA ATCTTACTTC GAAGCTACTA CAGCCTGTAA CTCTTTCAGG |
| 801 | AGGTACTCTA TCTTTAAAAC ATGGAGTGAC TCTGCAGACT CAGGCATTCA |
| 851 | CTCAACAGGC AGATTCTCGT CTCGAAATGG ACGTAGGAAC TACTCTAGAA |
| 901 | CCTGCTGATA CTAGCACCAT AAACAATTTG GTCATTAACA TCAGTTCTAT |
| 951 | AGACGGTGCA AAGAAGGCAA AAATAGAAAC CAAAGCTACG TCAAAAAATC |
| 1001 | TGACTTTATC TGGAACCATC ACTTTATTGG ACCCGACGGG CACGTTTTAT |
| 1051 | GAAAATCATA GTTTAAGAAA TCCTCAGTCC TACGACATCT TAGAGCTCAA |
| 1101 | AGCTTCTGGA ACTGTAACAA GCACCGCAGT GACTCCAGAT CCTATAATGG |
| 1151 | GTGAGAAATT CCATTACGGC TATCAGGGAA CTTGGGGCCC AATTGTTTGG |
| 1201 | GGGACAGGGG CTTCTACGAC TGCAACCTTC AACTGGACTA AAACTGGCTA |
| 1251 | TATTCCTAAT CCCGAGCGTA TCGGCTCTTT AGTCCCTAAT AGCTTATGGA |
| 1301 | ATGCATTTAT AGATATTAGC TCTCTCCATT ATCTTATGGA GACTGCAAAC |
| 1351 | GAAGGGTTGC AGGGAGACCG TGCTTTTTGG TGTGCTGGAT TATCTAACTT |
| 1401 | CTTCCATAAG GATAGTACAA AAACACGACG CGGGTTTCGC CATTTGAGTG |
| 1451 | GCGGTTATGT CATAGGAGGA AACCTACATA CTTGTTCAGA TAAGATTCTT |
| 1501 | AGTGCTGCAT TTTGTCAGCT CTTTGGAAGA GATAGAGACT ACTTTGTAGC |
| 1551 | TAAGAATCAA GGTACAGTCT ACGGAGGAAC TCTCTATTAC CAGCACAACG |
| 1601 | AAACCTATAT CTCTCTTCCT TGCAAACTAC GGCCTTGTTC GTTGTCTTAT |
| 1651 | GTTCCTACAG AGATTCCTGT TCTCTTTTCA GGAAACCTTA GCTACACCCA |
| 1701 | TACGGATAAC GATCTGAAAA CCAAGTATAC AACATATCCT ACTGTTAAAG |
| 1751 | GAAGCTGGGG GAATGATAGT TTCGCTTTAG AATTCGGTGG AAGAGCTCCG |
| 1801 | ATTTGCTTAG ATGAAAGTGC TCTATTTGAG CAGTACATGC CCTTCATGAA |
| 1851 | ATTGCAGTTT GTCTATGCAC ATCAGGAAGG TTTTAAAGAA CAGGGAACAG |
| 1901 | AAGCTCGTGA ATTTGGAAGT AGCCGTCTTG TGAATCTTGC CTTACCTATC |
| 1951 | GGGATCCGAT TTGATAAGGA ATCAGACTGC CAAGATGCAA CGTACAATCT |
| 2001 | AACTCTTGGT TATACTGTGG ATCTTGTTCG TAGTAACCCC GACTGTACGA |
| 2051 | CAACACTGCG AATTAGCGGT GATTCTTGGA AAACCTTCGG TACGAATTTG |
| 2101 | GCAAGACAAG CTTTAGTCCT TCGTGCAGGG AACCATTTTT GCTTTAACTC |
| 2151 | AAATTTTGAA GCCTTTAGCC AATTTTCTTT TGAATTGCGT GGGTCATCTC |
| 2201 | GCAATTACAA TGTAGACTTA GGAGCAAAAT ACCAATTCTA A |

The PSORT algorithm predicts a cytoplasmic location (0.274).

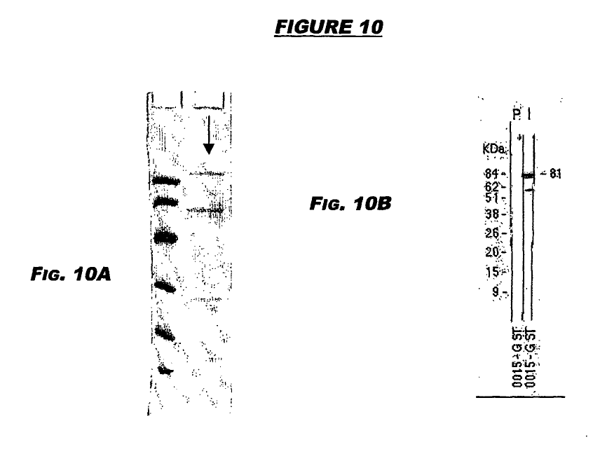

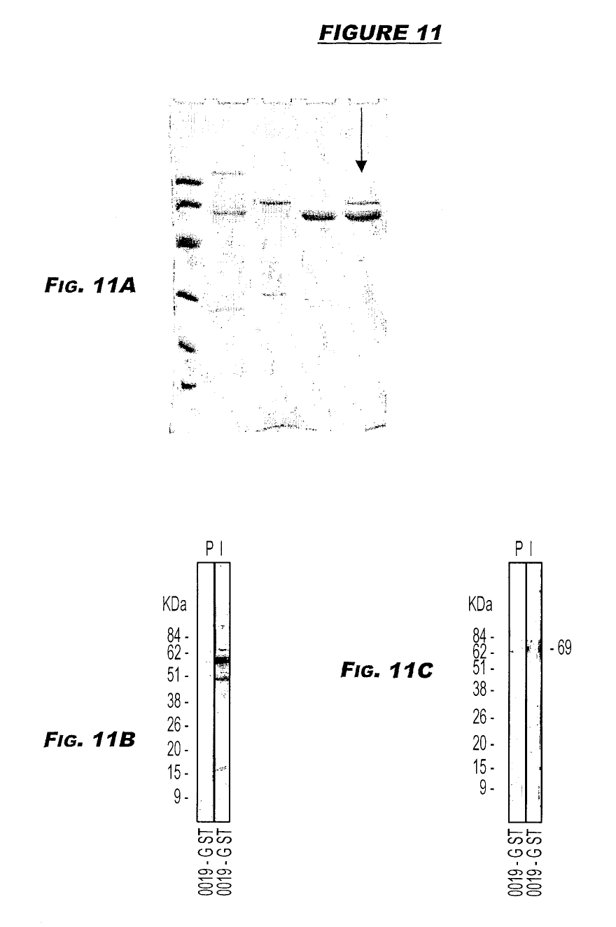

The protein was expressed in E. coli and purified as a GST-fusion product, as shown in FIG. 10A. The recombinant protein was used to immunize mice, whose sera were used in a Western blot (FIG. 10B) and for FACS analysis. A his-tagged protein was also expressed.

These experiments show that cp0015 is a useful immunogen. These properties are not evident from the sequence alone.

### Example 11

The following C. pneumoniae protein (PID 6172325) was expressed <SEQ ID 21; cp0019>:

| | |
|---|---|
| 1 | LQDSQDYSFV KLSPGAGGTI ITQDASQKPL EVAPSRPHYG YQGHWNVQVI |
| 51 | PGTGTQPSQA NLEWVRTGYL PNPERQGSLV PNSLWGSFVD QRAIQEIMVN |
| 101 | SSQILCQERG VWGAGIANFL HRDKINEHGY RHSGVGYLVG VGTHAFSDAT |
| 151 | INAAFCQLFS RDKDYVVSKN HGTSYSGVVF LEDTLEFRSP QGFYTDSSSE |
| 201 | ACCNQVVTID MQLSYSHRNN DMKTKYTTYP EAQGSWANDV FGLEFGATTY |
| 251 | YYPNSTFLFD YYSPFLRLQC TYAHQEDFKE TGGEVRHFTS GDLFNLAVPI |
| 301 | GVKFERFSDC KRGSYELTLA YVPDVIRKDP KSTATLASGA TWSTHGNNLS |
| 351 | RQGLQLRLGN HCLINPGIEV FSHGAIELRG SSRNYNTNLG GKYRF* |

This sequence is frame-shifted with respect to cp0018.

The cp0019 nucleotide sequence <SEQ ID 22> is:

| | |
|---|---|
| 1 | TTGCAAGACT CTCAAGACTA TAGCTTTGTA AAGTTATCTC CAGGAGCGGG |
| 51 | AGGGACTATA ATTACTCAAG ATGCTTCTCA GAAGCCTCTT GAAGTAGCTC |
| 101 | CTTCTAGACC ACATTATGGC TATCAAGGAC ATTGGAATGT GCAAGTCATC |
| 151 | CCAGGAACGG GAACTCAACC GAGCCAGGCA AATTTAGAAT GGGTGCGGAC |
| 201 | AGGATACCTT CCGAATCCCG AACGGCAAGG ATCTTTAGTT CCCAATAGCC |
| 251 | TGTGGGGTTC TTTTGTTGAT CAGCGTGCTA TCCAAGAAAT CATGGTAAAT |
| 301 | AGTAGCCAAA TCTTATGTCA GGAACGGGGA GTCTGGGGAG CTGGAATTGC |
| 351 | TAATTTCCTA CATAGAGATA AAATTAATGA GCACGGCTAT CGCCATAGCG |
| 401 | GTGTCGGTTA TCTTGTGGGA GTTGGCACTC ATGCTTTTTC TGATGCTACG |
| 451 | ATAAATGCGG CTTTTTGCCA GCTCTTCAGT AGAGATAAAG ACTACGTAGT |
| 501 | ATCCAAAAAT CATGGAACTA GCTACTCAGG GGTCGTATTT CTTGAGGATA |
| 551 | CCCTAGAGTT TAGAAGTCCA CAGGGATTCT ATACTGATAG CTCCTCAGAA |
| 601 | GCTTGCTGTA ACCAAGTCGT CACTATAGAT ATGCAGTTGT CTTACAGCCA |
| 651 | TAGAAATAAT GATATGAAAA CCAAATACAC GACATATCCA GAAGCTCAGG |
| 701 | GATCTTGGGC AAATGATGTT TTTGGTCTTG AGTTTGGAGC GACTACATAC |
| 751 | TACTACCCTA ACAGTACTTT TTTATTTGAT TACTACTCTC CGTTTCTCAG |
| 801 | GCTGCAGTGC ACCTATGCTC ACCAGGAAGA CTTCAAAGAG ACAGGAGGTG |
| 851 | AGGTTCGTCA CTTTACTAGC GGAGATCTTT TCAATTTAGC AGTTCCTATT |
| 901 | GGCGTGAAGT TTGAGAGATT TTCAGACTGT AAAAGGGGAT CTTATGAACT |
| 951 | TACCCTTGCT TATGTTCCTG ATGTGATTCG CAAAGATCCC AAGAGCACGG |
| 1001 | CAACATTGGC TAGTGGAGCT ACGTGGAGCA CCCACGGAAA CAATCTCTCC |
| 1051 | AGACAAGGAT TACAACTGCG TTTAGGGAAC CACTGTCTCA TAAATCCTGG |
| 1101 | AATTGAGGTG TTCAGTCACG GAGCTATTGA ATTGCGGGGA TCCTCTCGTA |
| 1151 | ATTATAACAT CAATCTCGGG GGTAAATACC GATTTTAA |

The PSORT algorithm predicts a cytoplasmic location (0.189).

The protein was expressed in E. coli and purified as a GST-fusion product, as shown in FIG. 11A. This protein was used to immunize mice, whose sera were used in a Western blot (FIG. 11B) and an immunoblot assay (FIG. 11C). A his-tagged protein was also expressed.

These experiments show that cp0019 is a useful immunogen. These properties are not evident from the sequence alone.

### Example 12

The following C. pneumoniae protein (PID 4376466) was expressed <SEQ ID 23; cp6466>:

| | |
|---|---|
| 1 | MRKISVGICI TILLSLSVVL QGCKESSHSS TSRGELAINI RDEPRSLDPR |
| 51 | QVRLLSEISL VKHIYEGLVQ ENNLSGNIEP ALAEDYSLSS DGLTYTFKLK |
| 101 | SAFWSNGDPL TAEDFIESWK QVATQEVSGI YAFALNPIKN VRKIQEGHLS |
| 151 | IDHFGVHSPN ESTLVVTLES PTSHFLKLLA LPVFFPVHKS QRTLQSKSLP |
| 201 | IASGAFYPKN IKQKQWIKLS KNPHYYNQSQ VETKTITIHF IPDANTAAKL |
| 251 | FNQGKLNWQG PPWGERIPQE TLSNLQSKGH LHSFDVAGTS WLTFNINKFP |
| 301 | LNNMKLREAL ASALDKEALV STIFLGRAKT ADHLLPTNIH SYPEHQKQEM |
| 351 | AQRQAYAKKL FKEALEELQI TAKDLEHLNL IFPVSSSASS LLVQLIREQW |
| 401 | KESLGFAIPI VGKEFALLQA DLSSGNFSLA TGGWFADFAD PMALFTIFAY |
| 451 | PSGVPPYAIN HKDFLEILQN IEQEQDHQKR SELVSQASLY LETFHIIEPI |
| 501 | YHDAFQFAMN KKLSNLGVSP TGVVDFRYAK EN* |

A predicted signal peptide is highlighted.

The cp6466 nucleotide sequence <SEQ ID 24> is:

| | |
|---|---|
| 1 | ATGCGCAAGA TATCAGTGGG AATCTGTATC ACCATTCTCC TTAGCCTCTC |
| 51 | CGTAGTCCTC CAAGGCTGCA AGGAGTCCAG TCACTCCTCT ACATCTCGGG |
| 101 | GAGAACTCGC TATTAATATA AGAGATGAAC CCCGTTCTTT AGATCCAAGA |
| 151 | CAAGTGCGAC TTCTTTCAGA AATCAGCCTT GTCAAACATA TCTATGAGGG |
| 201 | ATTAGTTCAA GAAAATAATC TTTCAGGAAA TATAGAGCCT GCTCTTGCAG |
| 251 | AAGACTACTC TCTTTCCTCG GACGGACTCA CTTATACTTT TAAACTGAAA |
| 301 | TCAGCTTTTT GGAGTAATGG CGACCCCTTA ACAGCTGAAG ACTTTATAGA |
| 351 | ATCTTGGAAA CAAGTAGCTA CTCAAGAAGT CTCAGGAATC TATGCTTTTG |
| 401 | CCTTGAATCC AATTAAAAAT GTACGAAAGA TCCAAGAGGG ACACCTCTCC |
| 451 | ATAGACCATT TTGGAGTGCA CTCTCCTAAT GAATCTACAC TTGTTGTTAC |
| 501 | CCTGGAATCC CCAACCTCGC ATTTCTTAAA ACTTTTAGCT CTTCCAGTCT |
| 551 | TTTTCCCCGT TCATAAATCT CAAAGAACCC TGCAATCCAA ATCTCTACCT |
| 601 | ATAGCAAGCG GAGCTTTCTA TCCTAAAAAT ATCAAACAAA AACAATGGAT |
| 651 | AAAACTCTCA AAAAACCCTC ACTACTATAA TCAAAGTCAG GTGGAAACTA |
| 701 | AAACGATTAC GATTCACTTC ATTCCCGATG CAAACACAGC AGCAAAACTA |
| 751 | TTTAATCAGG GAAAACTCAA TTGGCAAGGA CCTCCTTGGG GAGAACGCAT |
| 801 | TCCTCAAGAA ACCCTATCCA ATTTACAGTC TAAGGGGCAC TTACACTCTT |
| 851 | TTGATGTCGC AGGAACCTCA TGGCTCACCT TCAATATCAA TAAATTCCCC |
| 901 | CTCAACAATA TGAAGCTTAG AGAAGCCTTA GCATCAGCCT TAGATAAGGA |
| 951 | AGCTCTTGTC TCAACTATAT TCTTAGGCCG TGCAAAAACT GCCGATCATC |
| 1001 | TCCTACCTAC AAATATTCAT AGCTATCCCG AACATCAAAA ACAAGAGATG |
| 1051 | GCACAACGCC AAGCTTACGC TAAAAAACTC TTTAAAGAAG CTTTAGAAGA |
| 1101 | ACTCCAAATC ACTGCTAAAG ATCTCGAACA TCTTAATCTT ATCTTTCCCG |
| 1151 | TTTCCTCGTC AGCAAGTTCT TTACTAGTCC AACTTATACG AGAACAGTGG |
| 1201 | AAAGAAAGTT TAGGGTTCGC TATCCCTATT GTCGGAAAGG AATTTGCTCT |
| 1251 | TCTCCAAGCA GACCTATCTT CAGGGAACTT CTCTTTAGCT ACAGGAGGAT |
| 1301 | GGTTCGCAGA CTTTGCTGAT CCTATGGCAT TTCTAACGAT CTTTGCTTAT |
| 1351 | CCATCAGGAG TTCCTCCTTA TGCAATCAAC CATAAGGACT TCCTAGAAAT |
| 1401 | TCTACAAAAC ATAGAACAAG AGCAAGATCA CCAAAAACGC TCGGAATTAG |
| 1451 | TGTCGCAAGC TTCTCTTTAC CTAGAGACCT TTCATATTAT TGAGCCGATC |
| 1501 | TACCACGACG CATTTCAATT TGCTATGAAT AAAAAACTTT CTAATCTAGG |
| 1551 | AGTCTCACCA ACAGGAGTTG TGGACTTCCG TTATGCTAAG GAAAATTAG |

The PSORT algorithm predicts that the protein is an outer membrane lipoprotein (0.790).

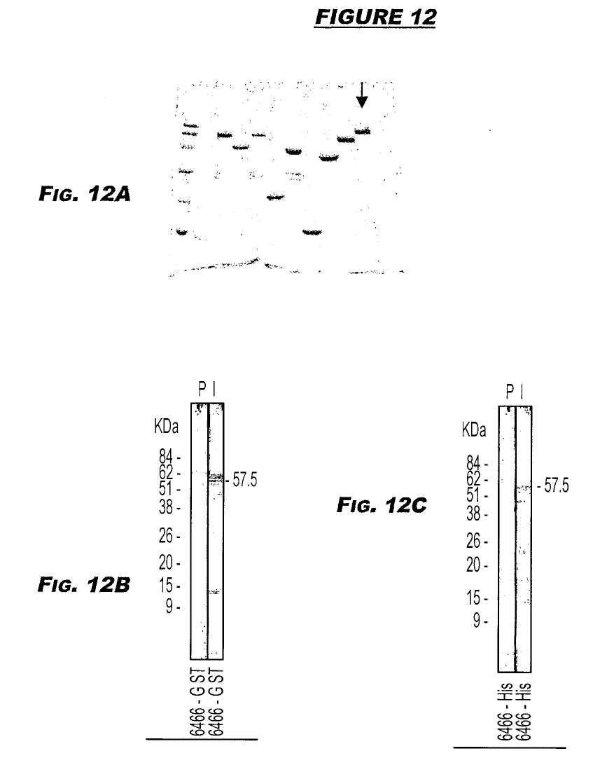

The protein was expressed in E. coli and purified both as a GST-fusion product and a His-tag fusion product. Purification of the protein as a GST-fusion product is shown in FIG. 12A. The recombinant proteins were used to immunize mice, whose sera were used in Western blots (FIGS. 12B and 12C). FACS analysis was also performed.

These experiments show that cp6466 is a useful immunogen. These properties are not evident from the sequence alone.

### Example 13

The following C. pneumoniae protein (PID 4376468) was expressed <SEQ ID 25; cp6468>:

| | |
|---|---|
| 1 | MFSRWITLFL LFISLTGCSS YSSKHKQSLI IPIHDDPVAF SPEQAKRAMD |
| 51 | LSIAQLLFDG LTRETHRESN DLELAIASRY TVSEDFCSYT FFIKDSALWS |
| 101 | DGTPITSEDI RNAWEYAQEN SPHIQIFQGL NFSTPSSNAI TIHLDSPNPD |
| 151 | FPKLLAFPAF AIFKPENPKL FSGPYTLVEY FPGHNIHLKK NPNYYDYHCV |
| 201 | SINSIKLLII PDIYTAIHLL NRGKVDWVGQ PWHQGIPWEL HKQSQYHYYT |
| 251 | YPVEGAFWLC LNTKSPHLND LQNRHRLATC IDKRSIIEEA LQGTQQPAET |
| 301 | LSRGAPQPNQ YKKQKPLTPQ EKLVLTYPSD ILRCQRIAEI LKEQWKAAGI |
| 351 | DLILEGLEYH LFVNKRKVQD YAIATQTGVA YYPGANLISE EDKLLQNFEI |
| 401 | IPIYYLSYDY LTQDFIEGVI YNASGAVDLK YTYFP* |

A predicted signal peptide is highlighted.

The cp6468 nucleotide sequence <SEQ ID 26> is:

| | |
|---|---|
| 1 | ATGTTTTCAC GATGGATCAC CCTCTTTTTA TTATTCATTA GCCTTACTGG |
| 51 | ATGCTCCTCC TACTCTTCAA AACATAAACA ATCTTTAATT ATTCCCATAC |
| 101 | ATGACGACCC TGTAGCTTTT TCTCCTGAAC AAGCAAAACG GGCCATGGAC |
| 151 | CTTTCTATTG CCCAACTTCT TTTTGATGGT CTGACTAGAG AAACTCATCG |
| 201 | CGAATCCAAT GATTTGGAAT TAGCGATTGC CAGTCGCTAT ACAGTCTCTG |
| 251 | AAGACTTTTG CTCTTATACG TTCTTTATCA AAGACAGCGC TTTATGGAGC |
| 301 | GACGGAACAC CAATCACCTC CGAAGATATC CGTAACGCTT GGGAGTATGC |
| 351 | ACAGGAGAAC TCTCCCCACA TACAGATCTT CCAAGGACTT AACTTCTCAA |
| 401 | CTCCTTCATC AAATGCAATT ACGATTCATC TCGACTCGCC CAACCCCGAT |
| 451 | TTTCCTAAGC TTCTTGCCTT TCCTGCATTT GCTATCTTTA AACCAGAAAA |
| 501 | CCCGAAGCTC TTTAGCGGTC CGTATACTCT TGTAGAGTAT TTCCCAGGGC |
| 551 | ATAACATTCA TTTAAAGAAA AACCCTAACT ATTACGACTA CCACTGCGTC |
| 601 | TCCATCAACT CCATCAAACT GCTCATTATT CCTGATATAT ATACAGCCAT |
| 651 | CCACCTCCTA AACAGAGGCA AGGTGGACTG GGTAGGACAA CCCTGGCATC |
| 701 | AAGGGATTCC TTGGGAGCTC CATAAACAAT CGCAATATCA CTACTACACC |
| 751 | TATCCTGTAG AAGGTGCCTT CTGGCTTTGT CTAAATACAA AATCCCCACA |
| 801 | CTTAAATGAT CTTCAAAACA GACATAGACT CGCTACTTGT ATTGATAAAC |
| 851 | GTTCTATCAT TGAAGAAGCT CTTCAAGGAA CCCAACAACC AGCGGAAACA |
| 901 | CTGTCCCGAG GAGCTCCACA ACCAAATCAA TATAAAAAAC AAAAGCCTCT |
| 951 | AACTCCACAA GAAAAACTCG TGCTTACCTA TCCCTCAGAT ATTCTAAGAT |
| 1001 | GCCAACGCAT AGCAGAAATC TTAAAGGAAC AATGGAAAGC TGCTGGAATA |
| 1051 | GATTTAATCC TTGAAGGACT CGAATACCAT CTGTTTGTTA ACAAACGAAA |
| 1101 | AGTCCAAGAC TACGCCATAG CAACACAGAC TGGAGTTGCT TATTACCCAG |
| 1151 | GAGCAAATCT AATTTCTGAA GAAGACAAGC TCCTGCAAAA CTTTGAGATT |
| 1201 | ATCCCGATCT ACTATCTGAG CTATGACTAT CTCACTCAAG ATTTTATAGA |
| 1251 | GGGAGTAATC TATAATGCTT CTGGAGCTGT AGATCTCAAA TATACCTATT |
| 1301 | TCCCCTAG |

The PSORT algorithm predicts that this protein is an outer membrane lipoprotein (0.790).

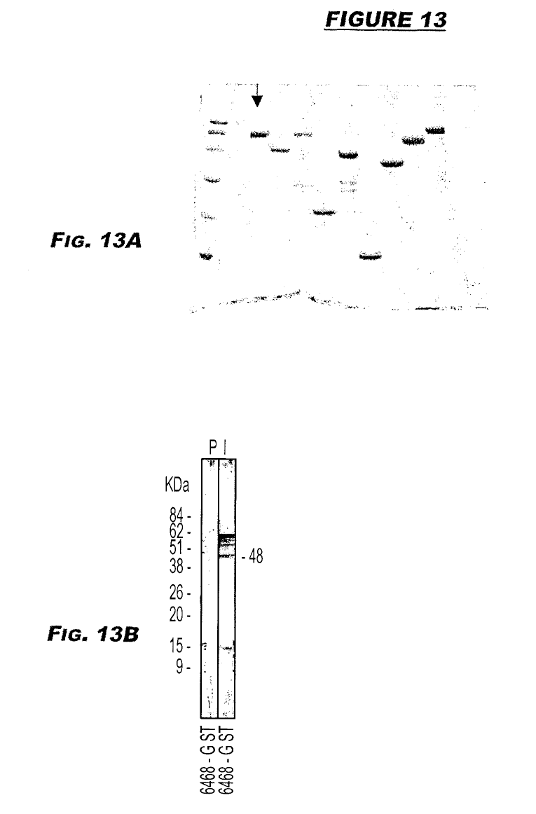

The protein was expressed in E. coli and purified as a GST-fusion product, as shown in FIG. 13A. The recombinant protein was used to immunize mice, whose sera were used in a Western blot (FIG. 13B) and for FACS analysis. A his-tagged protein was also expressed.

These experiments show that cp6468 is a useful immunogen. These properties are not evident from the sequence alone.

### Example 14

The following C. pneumoniae protein (PID 4376469) was expressed <SEQ ID 27; cp6469>:

| | |
|---|---|
| 1 | MKMHRLKPTL KSLIPNLLFL LLTLSSCSKQ KQEPLGKHLV IAMSHDLADL |
| 51 | DPRNAYLSRD ASLAKALYEG LTRETDQGIA LALAESYTLS KDHKVYTFKL |
| 101 | RPSVWSDGTP LTAYDFEKSI KQLYFEEFSP SIHTLLGVIK NSSAIHNAQK |
| 151 | SLETLGIQAK DDLTLVITLE QPFPYFLTLI ARPVFSPVHH TLRESYKKGT |
| 201 | PESTYISNGP FVLKKHEHQN YLILEKNPHY YDHESVKLDR VTLKIIPDAS |
| 251 | TATKLFKSKS IDWIGSPWSA PISNEDQKVL SQEKILTYSV SSTTLLIYNL |
| 301 | QKPLIQNKAL RKAIAHAIDR KSILRLVPSG QEAVTLVPPN LSQLNLQKEI |
| 351 | STEERQTKAR AYFQEAKETL SEKELAELSI LYPIDSSNSS IIAQEIQRQL |
| 401 | KDTLGLKIKI QGMEYHCFLK KRRQGDFFIA TGGWIAEYVS PVAFLSILGN |
| 451 | PRDLTQWRNS DYEKTLEKLY LPHAYKENLK RAEMIIEEET PIIPLYHGKY |
| 501 | IYAIHPKIQN TFGSLLGHTD LKNIDILS* |

A predicted signal peptide is highlighted.

The cp6469 nucleotide sequence <SEQ ID 28> is:

| | |
|---|---|
| 1 | ATGAAGATGC ATAGGCTTAA ACCTACCTTA AAAAGTCTGA TCCCTAATCT |
| 51 | TCTTTTCTTA TTGCTCACTC TTTCAAGCTG CTCAAAGCAA AAACAAGAAC |
| 101 | CCTTAGGAAA ACATCTCGTT ATTGCGATGA GCCATGATCT CGCCGACCTA |
| 151 | GATCCTCGCA ATGCCTATTT AAGCAGAGAT GCTTCCCTAG CAAAAGCCCT |
| 201 | CTATGAAGGA CTGACAAGAG AAACTGATCA AGGAATCGCA CTGGCTCTTG |
| 251 | CAGAAAGTTA TACCCTGTCA AAAGATCATA AGGTCTATAC CTTTAAACTC |
| 301 | AGACCTTCTG TGTGGAGCGA TGGCACTCCA CTCACTGCTT ATGACTTTGA |
| 351 | AAAATCTATA AAACAACTGT ACTTCGAAGA ATTTTCACCT TCCATACATA |
| 401 | CTTTACTCGG CGTGATTAAA AATTCTTCGG CAATCCACAA TGCTCAAAAA |
| 451 | TCTCTGGAAA CTCTTGGGAT ACAGGCAAAA GATGATCTTA CTTTGGTGAT |
| 501 | TACCCTAGAG CAACCTTTCC CATACTTTCT CACACTTATC GCTCGCCCCG |
| 551 | TATTCTCCCC TGTTCATCAC ACCCTTAGGG AATCCTATAA GAAAGGAACA |
| 601 | CCCCCATCCA CATACATCTC CAATGGGCCC TTTGTCTTAA AAAAACATGA |
| 651 | ACACCAAAAC TACTTAATTT TAGAAAAAAA TCCTCACTAC TATGATCATG |
| 701 | AATCAGTAAA GTTAGACCGA GTCACCTTAA AAATTATCCC AGACGCCTCC |
| 751 | ACAGCCACGA AACTTTTCAA AAGTAAATCT ATAGATTGGA TTGGCTCACC |
| 801 | TTGGAGCGCT CCGATATCTA ACGAAGACCA AAAAGTTCTC TCCCAAGAAA |
| 851 | AGATTCTTAC CTATTCTGTT TCAAGCACCA CCCTTCTTAT CTATAACCTG |
| 901 | CAAAAACCTC TAATACAAAA TAAAGCCCTC AGGAAAGCCA TTGCTCATGC |
| 951 | TATTGATAGA AAATCTATCT TAAGACTCGT GCCTTCAGGA CAAGAAGCTG |
| 1001 | TAACTCTAGT TCCCCCAAAT CTTTCACAAC TCAATCTTCA AAAAGAGATC |
| 1051 | TCAACAGAAG AACGACAAAC AAAAGCCAGA GCATATTTTC AAGAAGCTAA |
| 1101 | AGAAACACTT TCTGAAAAAG AACTCGCAGA ACTCAGCATC CTCTATCCTA |
| 1151 | TAGATTCCTC GAATTCCTCC ATCATAGCTC AAGAAATCCA AAGACAACTT |
| 1201 | AAAGATACCT TAGGATTGAA AATCAAAATC CAAGGCATGG AGTACCACTG |
| 1251 | CTTTTTAAAG AAACGTCGTC AAGGAGATTT CTTCATAGCG ACAGGAGGAT |
| 1301 | GGATTGCGGA ATACGTAAGC CCCGTAGCCT TCCTATCTAT TCTAGGCAAC |
| 1351 | CCCAGAGACC TCACACAATG GAGAAACAGT GATTACGAAA AGACTTTAGA |
| 1401 | GAAACTCTAT CTCCCTCATG CCTACAAAGA GAATTTAAAA CGCGCAGAAA |
| 1451 | TGATAATAGA AGAAGAAACC CCGATTATCC CCCTGTATCA CGGCAAATAT |
| 1501 | ATTTACGCTA TACATCCTAA AATCCAGAAT ACATTCGGAT CTCTTCTAGG |
| 1551 | CCACACAGAT CTCAAAAATA TCGATATCTT AAGTTAG |

The PSORT algorithm predicts a periplasmic location (0.934).

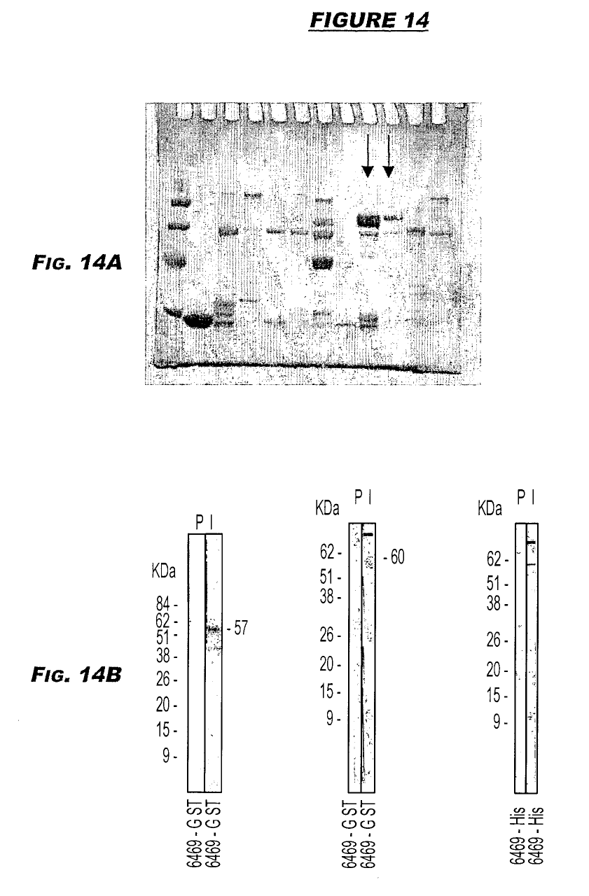

The protein was expressed in E. coli and purified as a GST-fusion product, as shown in FIG. 14A. The recombinant protein was used to immunize mice, whose sera were used in a Western blot (FIG. 14B) and for FACS analysis. A his-tagged protein was also expressed.

These experiments show that cp6469 is a useful immunogen. These properties are not evident from the sequence alone.

### Example 15

The following C. pneumoniae protein (PID 4376602) was expressed <SEQ ID 29; cp6602>:

| | |
|---|---|
| 1 | MAASGGTGGL GGTQGVNLAA VEAAAAKADA AEVVASQEGS EMNMIQQSQD |
| 51 | LTNPAAATRT KKKEEKFQTL ESRKKGEAGK AEKKSESTEE KPDTDLADKY |
| 101 | ASGNSEISGQ ELRGLRDAIG DDASPEDILA LVQEKIKDPA LQSTALDYLV |
| 151 | QTTPPSQGKL KEALIQARNT HTEQFGRTAI GAKNILFASQ EYADQLNVSP |
| 201 | SGLRSLYLEV TGDTHTCDQL LSMLQDRYTY QDMAIVSSFL MKGMATELKR |
| 251 | QGPYVPSAQL QVLMTETRNL QAVLTSYDYF ESRVPILLDS LKAEGIQTPS |
| 301 | DLNFVKVAES YHKIINDKFP TASKVEREVR NLIGDDVDSV TGVLNLFFSA |
| 351 | LRQTSSRLFS SADKRQQLGA MIANALDAVN INNEDYPKAS DFPKPYPWS* |

The cp6602 nucleotide sequence <SEQ ID 30> is:

| | |
|---|---|
| 1 | ATGGCAGCAT CAGGAGGCAC AGGTGGTTTA GGAGGCACTC AGGGTGTCAA |
| 51 | CCTTGCAGCT GTAGAAGCTG CAGCTGCAAA AGCAGATGCA GCAGAAGTTG |
| 101 | TAGCCAGCCA AGAAGGTTCT GAGATGAACA TGATTCAACA ATCTCAGGAC |
| 151 | CTGACAAATC CCGCAGCAGC AACACGCACG AAAAAAAAGG AAGAGAAGTT |
| 201 | TCAAACTCTA GAATCTCGGA AAAAAGGAGA AGCTGGAAAG GCTGAGAAAA |
| 251 | AATCTGAATC TACAGAAGAG AAGCCTGACA CAGATCTTGC TGATAAGTAT |
| 301 | GCTTCTGGGA ATTCTGAAAT CTCTGGTCAA GAACTTCGCG GCCTGCGTGA |
| 351 | TGCAATAGGA GACGATGCTT CTCCAGAAGA CATTCTTGCT CTTGTACAAG |
| 401 | AGAAAATTAA AGACCCAGCT CTGCAATCCA CAGCTTTGGA CTACCTGGTT |
| 451 | CAAACGACTC CACCCTCCCA AGGTAAATTA AAAGAAGCGC TTATCCAAGC |
| 501 | AAGGAATACT CATACGGAGC AATTCGGACG AACTGCTATT GGTGCGAAAA |
| 551 | ACATCTTATT TGCCTCTCAA GAATATGCAG ACCAACTGAA TGTTTCTCCT |
| 601 | TCAGGGCTTC GCTCTTTGTA CTTAGAAGTG ACTGGAGACA CACATACCTG |
| 651 | TGATCAGCTA CTTTCTATGC TTCAAGACCG CTATACCTAC CAAGATATGG |
| 701 | CTATTGTCAG CTCCTTTCTA ATGAAAGGAA TGGCAACAGA ATTAAAAAGG |
| 751 | CAGGGTCCCT ACGTACCCAG TGCGCAACTA CAAGTTCTCA TGACAGAAAC |
| 801 | TCGTAACCTG CAAGCAGTTC TTACCTCGTA CGATTACTTT GAAAGTCGCG |
| 851 | TTCCTATTTT ACTCGATAGC TTAAAAGCTG AGGGAATCCA AACTCCTTCT |
| 901 | GATCTAAACT TTGTGAAGGT AGCTGAGTCC TACCATAAAA TCATTAACGA |
| 951 | TAAGTTCCCA ACAGCATCTA AAGTAGAACG AGAAGTCCGC AATCTCATAG |
| 1001 | GAGACGATGT TGATTCTGTG ACCGGTGTCT TGAACTTATT CTTTTCTGCT |
| 1051 | TTACGTCAAA CGTCGTCACG CCTTTTCTCT TCAGCAGACA AACGTCAGCA |
| 1101 | ATTAGGAGCT ATGATTGCTA ATGCTTTAGA TGCTGTAAAT ATAAACAATG |
| 1151 | AAGATTATCC CAAAGCATCA GACTTCCCTA AACCCTATCC TTGGTCATGA |

The PSORT algorithm predicts a cytoplasmic location (0.080).

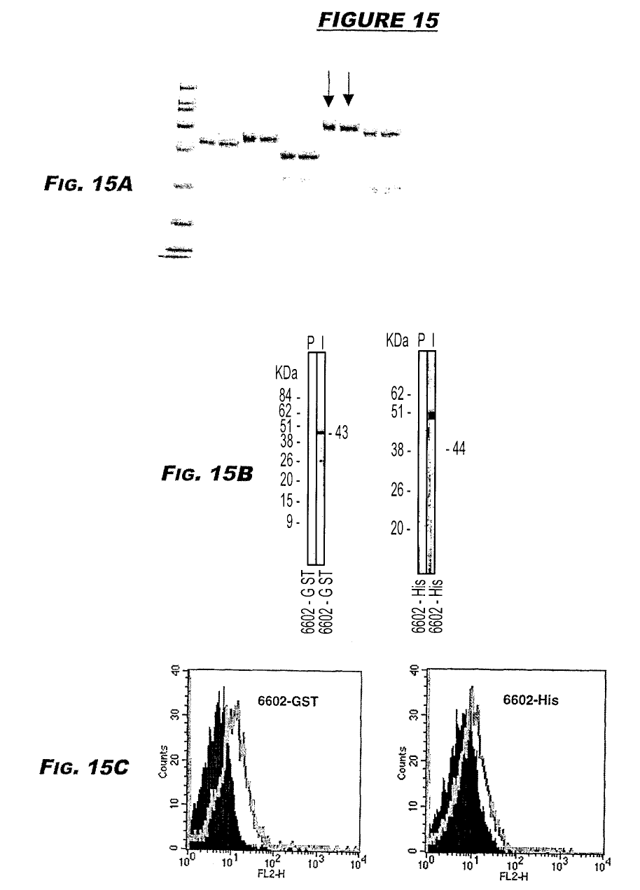

The protein was expressed in E. coli and purified as both a His-tag and a GST-fusion product, as shown in FIG. 15A. The recombinant proteins were used to immunize mice, whose sera were used in a Western blot (FIG. 15B) and for FACS analysis (FIG. 15C).

The cp6602 protein was also identified in the 2D-PAGE experiment (Cpn0324).

These experiments show that cp6602 is a surface-exposed and immunoaccessible protein, and that it is a useful immunogen. These properties are not evident from the sequence alone.

### Example 16

The following C. pneumoniae protein (PID 4376727) was expressed <SEQ ID 31; cp6727>:

| | |
|---|---|
| 1 | MKYSLPWLLT SSALVFSLHP LMAANTDLSS SDNYENGSSG SAAFTAKETS |
| 51 | DASGTTYTLT SDVSITNVSA ITPADKSCFT NTGGALSFVG ADHSLVLQTI |
| 101 | ALTHDGAAIN NTNTALSFSG FSSLLIDSAP ATGTSGGKGA ICVTNTEGGT |
| 151 | ATFTDNASVT LQKNTSEKDG AAVSAYSIDL AKTTTAALLD QNTSTKNGGA |
| 201 | LCSTANTTVQ GNSGTVTFSS NTATDKGGGI YSKEKDSTLD ANTGVVTFKS |
| 251 | NTAKTGGAWS SDDNLALTGN TQVLFQENKT TGSAAQANNP EGCGGAICCY |
| 301 | LATATDKTGS AISQNQEMSF TSNTTTANGG AIYATKCTLD GNTTLTFDQN |
| 351 | TATAGCGGAI YTETEDFSLK GSTGTVTFST NTAKTGGALY SKGNSSLTGN |
| 401 | TNLLFSGNKA TGPSNSSANQ EGCGGAILAF IDSGSVSDKT GLSIANNQEV |
| 451 | SLTSNAATVS GGAIYATKCT LTGNGSLTFD GNTAGTSGGA IYTETEDFTL |
| 501 | TGSTGTVTFS TNTAKTGGAL YSKGNNSLSG NTNLLFSGNK ATGPSNSSAN |
| 551 | QEGCGGAILS FLESASVSTK KGLWIEDNEN VSLSGNTATV SGGAIYATKC |
| 601 | ALHGNTTLTF DGNTAETAGG AIYTETEDFT LTGSTGTVTF STNTAKTAGA |
| 651 | LHTKGNTSFT KNKALVFSGN SATATATTTT DQEGCGGAIL CNISESDIAT |
| 701 | KSLTLTENES SLFINNTAKR SGGGIYAPKC VISGSESINF DGNTAETSGG |
| 751 | AIYSKNLSIT ANGPVSFTNN SGGKGGAIYI ADSGELSLEA IDGDITFSGN |
| 801 | RATEGTSTPN SIHLGAGAKI TKLAAAPGHT IYFYDPITME APASGGTIEE |
| 851 | LVINPVVKAI VPPPQPKNGP IASVPVVPVA PANPNTGTIV FSSGKLPSQD |
| 901 | ASIPANTTTI LNQKINLAGG VNNLKEGATL QVYSFTQQPD STVFMDAGTT |
| 951 | LETTTTNNTD GSIDLKNLSV NLDALDGKRM ITIAVNSTSG GLKISGDLFK |
| 1001 | HNNEGSFYDN PGLKANLNLP FLDLSSTSGT VNLDDFNPIP SSMAAPDYGY |
| 1051 | QGSWTLVPKV GAGGKVTLVA EWQALGYTPK PELRATLVPN SLWNAYVNIH |
| 1101 | SIQQEIATAM SDAPSHPGIW IGGIGNAFHQ DKQKENAGFR LISRGYIVGG |
| 1151 | SMTTPQEYTF AVAFSQLFGK SKDYVVSDIK SQVYAGSLCA QSSYVIPLHS |
| 1201 | SLRRHVLSKV LPELPGETPL VLHGQVSYGR NHHNMTTKLA NNTQGKSDWD |
| 1251 | SHSFAVEVGG SLPVDLNYRY LTSYSPYVKL QVVSVNQKGF QEVAADPRIF |
| 1301 | DASHLVNVSI PMGLTFKHES AKPPSALLLT LGYAVDAYRD HPHCLTSLTN |
| 1351 | GTSWSTFATN LSRQAFFAEA SGHLKLLHGL DCFASGSCEL RSSSRSYNAN |
| 1401 | CGTRYSF* |

A predicted signal peptide is highlighted.

The cp6727 nucleotide sequence <SEQ ID 32> is:

| | |
|---|---|
| 1 | ATGAAATATT CTTTACCTTG GCTACTTACC TCTTCGGCTT TAGTTTTCTC |
| 51 | CCTACATCCA CTAATGGCTG CTAACACGGA TCTCTCATCA TCCGATAACT |
| 101 | ATGAAAATGG TAGTAGTGGT AGCGCAGCAT TCACTGCCAA GGAAACTTCG |
| 151 | GATGCTTCAG GAACTACCTA CACTCTCACT AGCGATGTTT CTATTACGAA |
| 201 | TGTATCTGCA ATTACTCCTG CAGATAAAAG CTGTTTTACA AACACAGGAG |
| 251 | GAGCATTGAG TTTTGTTGGA GCTGATCACT CATTGGTTCT GCAAACCATA |
| 301 | GCGCTTACGC ATGATGGTGC TGCAATTAAC AATACCAACA CAGCTCTTTC |
| 351 | TTTCTCAGGA TTCTCGTCAC TCTTAATCGA CTCAGCTCCA GCAACAGGAA |
| 401 | CTTCGGGCGG CAAGGGTGCT ATTTGTGTGA CAAATACAGA GGGAGGTACT |
| 451 | GCGACTTTTA CTGACAATGC CAGTGTCACC CTCCAAAAAA ATACTTCAGA |
| 501 | AAAAGATGGA GCTGCAGTTT CTGCCTACAG CATCGATCTT GCTAAGACTA |
| 551 | CGACAGCAGC TCTCTTAGAT CAAAATACTA GCACAAAAAA TGGCGGGGCC |
| 601 | CTCTGTAGTA CAGCAAACAC TACAGTCCAA GGAAACTCAG GAACGGTGAC |
| 651 | CTTCTCCTCA AATACTGCTA CAGATAAAGG TGGGGGGATC TACTCAAAAG |
| 701 | AAAAGGATAG CACGCTAGAT GCCAATACAG GAGTCGTTAC CTTCAAATCT |
| 751 | AATACTGCAA AGACGGGGGG TGCTTGGAGC TCTGATGACA ATCTTGCTCT |
| 801 | TACCGGCAAC ACTCAAGTAC TTTTTCAGGA AAATAAAACA ACCGGCTCAG |
| 851 | CAGCACAGGC AAATAACCCG GAAGGTTGTG GTGGGGCAAT CTGTTGTTAT |
| 901 | CTTGCTACAG CAACAGACAA AACTGGATTA GCCATTTCTC AGAATCAAGA |
| 951 | AATGAGCTTC ACTAGTAATA CAACAACTGC GAATGGTGGA GCGATCTACG |
| 1001 | CTACTAAATG TACTCTGGAT GGAAACACAA CTCTTACCTT CGATCAGAAT |
| 1051 | ACTGCGACAG CAGGATGTGG CGGAGCTATC TATACAGAAA CTGAAGATTT |
| 1101 | TTCTCTTAAG GGAAGTACGG GAACCGTGAC CTTCAGCACA AATACAGCAA |
| 1151 | AGACAGGCGG CGCCTTATAT TCTAAAGGAA ACAGCTCGCT GACTGGAAAT |
| 1201 | ACCAACCTGC TCTTTTCAGG GAACAAAGCT ACGGGCCCGA GTAATTCTTC |
| 1251 | AGCAAATCAA GAGGGTTGCG GTGGGGCAAT CCTAGCCTTT ATTGATTCAG |
| 1301 | GATCCGTAAG CGATAAAACA GGACTATCGA TTGCAAACAA CCAAGAAGTC |
| 1351 | AGCCTCACTA GTAATGCTGC AACAGTAAGT GGTGGTGCGA TCTATGCTAC |
| 1401 | CAAATGTACT CTAACTGGAA ACGGCTCCCT GACCTTTGAC GGCAATACTG |
| 1451 | CTGGAACTTC AGGAGGGGCG ATCTATACAG AAACTGAAGA TTTTACTCTT |
| 1501 | ACAGGAAGTA CAGGAACCGT GACCTTCAGC ACAAATACAG CAAAGACAGG |
| 1551 | CGGCGCCTTA TATTCTAAAG GCAACAACTC TCTGTCTGGT AATACCAACC |
| 1601 | TGCTCTTTTC AGGGAACAAA GCTACGGGCC CGAGTAATTC TTCAGCAAAT |
| 1651 | CAAGAGGGTT GCGGTGGGGC AATCCTATCG TTTCTTGAGT CAGCATCTGT |
| 1701 | AAGTACTAAA AAAGGACTCT GGATTGAAGA TAACGAAAAC GTGAGTCTCT |
| 1751 | CTGGTAATAC TGCAACAGTA AGTGGCGGTG CGATCTATGC GACCAAGTGT |
| 1801 | GCTCTGCATG GAAACACGAC TCTTACCTTT GATGGCAATA CTGCCGAAAC |
| 1851 | TGCAGGAGGA GCGATCTATA CAGAAACCGA AGATTTTACT CTTACGGGAA |
| 1901 | GTACGGGAAC CGTGACCTTC AGCACAAATA CAGCAAAGAC AGCAGGGGCT |
| 1951 | CTACATACTA AAGGAAATAC TTCCTTTACC AAAAATAAGG CTCTTGTATT |
| 2001 | TTCTGGAAAT TCAGCAACAG CAACAGCAAC AACAACTACA GATCAAGAAG |
| 2051 | GTTGTGGTGG AGCGATCCTC TGTAATATCT CAGAGTCTGA CATAGCTACA |
| 2101 | AAAAGCTTAA CTCTTACTGA AAATGAGAGT TTAAGTTTCA TTAACAATAC |
| 2151 | GGCAAAAAGA AGTGGTGGTG GTATTTATGC TCCTAAGTGT GTAATCTCAG |
| 2201 | GCAGTGAATC CATAAACTTT GATGGCAATA CTGCTGAAAC TTCGGGAGGA |
| 2251 | GCGATTTATT CGAAAAACCT TTCGATTACA GCTAACGGTC CTGTCTCCTT |
| 2301 | TACCAATAAT TCTGGAGGCA AGGGAGGCGC CATTTATATA GCCGATAGCG |
| 2351 | GAGAACTTTC CTTAGAGGCT ATTGATGGGG ATATTACTTT CTCAGGGAAC |
| 2401 | CGAGCGACTG AGGGAACTTC AACTCCCAAC TCGATCCATT TAGGTGCAGG |
| 2451 | GGCTAAGATC ACTAAGCTTG CAGCAGCTCC TGGTCATACG ATTTATTTTT |
| 2501 | ATGATCCTAT TACGATGGAA GCTCCTGCAT CTGGAGGAAC AATAGAGGAG |
| 2551 | TTAGTCATCA ATCCTGTTGT CAAAGCTATT GTTCCTCCTC CCCAACCAAA |
| 2601 | AAATGGTCCT ATAGCTTCAG TGCCTGTAGT CCCTGTAGCA CCTGCAAACC |
| 2651 | CAAACACGGG AACTATAGTA TTTTCTTCTG GAAAACTCCC CAGTCAAGAT |
| 2701 | GCCTCGATTC CTGCAAATAC TACCACCATA CTGAACCAGA AGATCAACTT |
| 2751 | AGCAGGAGGA AATGTCGTTT TAAAAGAAGG AGCCACCCTA CAAGTATATT |
| 2801 | CCTTCACACA GCAGCCTGAT TCTACAGTAT TCATGGATGC AGGAACGACC |
| 2851 | TTAGAGACCA CGACAACTAA CAATACAGAT GGCAGCATCG ATCTAAAGAA |
| 2901 | TCTCTCTGTA AATCTGGATG CTTTAGATGG CAAGCGTATG ATAACGATTG |
| 2951 | CCGTAAACAG CACAAGTGGG GGATTAAAAA TCTCAGGGGA TCTGAAATTC |
| 3001 | CATAACAATG AAGGAAGTTT CTATGACAAT CCTGGGTTGA AAGCAAACTT |
| 3051 | AAATCTTCCT TTCTTAGATC TTTCTTCTAC TTCAGGAACT GTAAATTTAG |
| 3101 | ACGACTTCAA TCCGATTCCT TCTAGCATGG CTGCTCCGGA TTATGGGTAT |
| 3151 | CAAGGGAGTT GGACTCTGGT TCCTAAAGTA GGAGCTGGAG GGAAGGTGAC |
| 3201 | TTTGGTCGCG GAATGGCAAG CGTTAGGATA CACTCCTAAA CCAGAGCTTC |
| 3251 | GTGCGACTTT AGTTCCTAAT AGCCTTTGGA ATGCTTATGT AAACATCCAT |
| 3301 | TCTATACAGC AGGAGATCGC CACTGCGATG TCGGACGCTC CCTCACATCC |
| 3351 | AGGGATTTGG ATTGGAGGTA TTGGCAACGC CTTCCATCAA GACAAGCAAA |
| 3401 | AGGAAAATGC AGGATTCCGT TTGATTTCCA GAGGTTATAT TGTTGGTGGC |
| 3451 | AGCATGACCA CCCCTCAAGA ATATACCTTT GCTGTTGCAT TCAGCCAACT |
| 3501 | CTTTGGCAAA TCTAAGGATT ACGTAGTCTC GGATATTAAA TCTCAAGTCT |
| 3551 | ATGCAGGATC TCTCTGTGCT CAGAGCTCTT ATGTCATTCC CCTGCATAGC |
| 3601 | TCATTACGTC GCCACGTCCT CTCTAAGGTC CTTCCAGAGC TCCCAGGAGA |
| 3651 | AACTCCCCTT GTTCTCCATG GTCAAGTTTC CTATGGAAGA AACCACCATA |
| 3701 | ATATGACGAC AAAGCTTGCG AACAACACAC AAGGGAAATC AGACTGGGAC |
| 3751 | AGCCATAGCT TCGCTGTTGA AGTCGGTGGT TCTCTTCCTG TAGATCTAAA |
| 3801 | CTACAGATAC CTTACCAGCT ACTCTCCCTA TGTGAAACTC CAAGTTGTGA |
| 3851 | GTGTAAATCA AAAAGGATTC CAAGAGGTTG CTGCTGATCC ACGTATCTTT |
| 3901 | GACGCTAGCC ATCTGGTCAA CGTGTCTATC CCTATGGGAC TCACCTTCAA |
| 3951 | ACACGAATCA GCAAAGCCCC CCAGTGCTTT GCTTCTTACT TTAGGTTACG |
| 4001 | CTGTAGATGC TTACCGGGAT CACCCTCACT GCCTGACCTC CTTAACAAAT |
| 4051 | GGCACCTCGT GGTCTACGTT TGCTACAAAC TTATCACGAC AAGCTTTCTT |
| 4101 | TGCTGAGGCT TCTGGACATC TGAAGTTACT TCATGGTCTT GACTGCTTCG |
| 4151 | CTTCTGGAAG TTGTGAACTG CGCAGCTCCT CAAGAAGCTA TAATGCAAAC |
| 4201 | TGTGGAACTC GTTATTCTTT CTAA |

The PSORT algorithm predicts an outer membrane location (0.915).

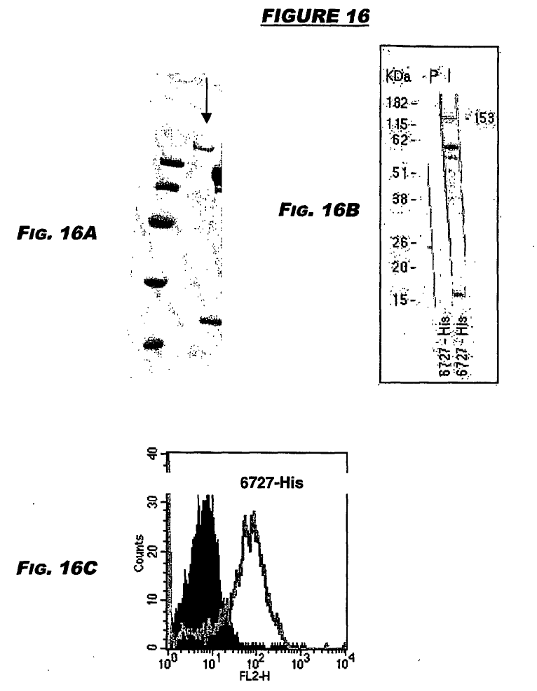

The protein was expressed in E. coli and purified as a his-tag product, as shown in FIG. 16A. The recombinant protein was used to immunize mice, whose sera were used in a Western blot (FIG. 16B) and for FACS analysis (FIG. 16C). A GST-fusion protein was also expressed.

The cp6727 protein was also identified in the 2D-PAGE experiment (Cpn0444).

These experiments show that cp6727 is a surface-exposed and immunoaccessible protein, and that it is a useful immunogen. These properties are not evident from the sequence alone.

### Example 17

The following C. pneumoniae protein (PID 4376731) was expressed <SEQ ID 33; cp6731>:

| | |
|---|---|
| 1 | MKSSLHWFLI SSSLALPLSL NFSAFAAVVE INLGPTNSFS GPGTYTPPAQ |
| 51 | TTNADGTIYN LTGDVSITNA GSPTALTASC FKETTGNLSF QGHGYQFLLQ |
| 101 | NIDAGANCTF TNTAANKLLS FSGFSYLSLI QTTNATTGTG AIKSTGACSI |
| 151 | QSNYSCYFGQ NFSNDNGGAL QGSSISLSLN PNLTFAKNKA TQKGGALYST |
| 201 | GGITINNTLN SASFSENTAA NNGGAIYTEA SSFISSNKAI SFINNSVTAT |
| 251 | SATGGAIYCS STSAPKPVLT LSDNGELNFI GNTAITSGGA IYTDNLVLSS |
| 301 | GGPTLFKNNS AIDTAAPLGG AIAIADSGSL SLSALGGDIT FEGNTVVKGA |
| 351 | SSSQTTTRNS INIGNTNAKI VQLRASQGNT IYFYDPITTS ITAALSDALN |
| 401 | LNGPDLAGNP AYQGTIVFSG EKLSEAEAAE ADNLKSTIQQ PLTLAGGQLS |
| 451 | LKSGVTLVAK SFSQSPGSTL LMDAGTTLET ADTITINNLV LNVDSLKETK |
| 501 | KATLKATQAS QTVTLSGSLS LVDPSGNVYE DVSWNNPQVF SCLTLTADDP |
| 551 | ANIHITDLAA DPLEKNPIHW GYQGNWALSW QEDTATKSKA ATLTWTKTGY |
| 601 | NPNPERRGTL VANTLWGSFV DVRSIQQLVA TKVRQSQETR GIWCEGISNF |
| 651 | FHKDSTKINK GFRHISAGYV VGATTTLASD NLITAAFCQL FGKDRDHFIN |
| 701 | KNRASAYAAS LHLQHLATLS SPSLLRYLPG SESEQPVLFD AQISYIYSKN |
| 751 | TMKTYYTQAP KGESSWYNDG CALELASSLP HTALSHEGLF HAYFPFIKVE |
| 801 | ASYIHQDSFK ERNTTLVRSF DSGDLINVSV PIGITFERFS RNERASYEAT |
| 851 | VIYVADVYRK NPDCTTALLI NNTSWKTTGT NLSRQAGIGR AGIFYAFSPN |
| 901 | LEVTSNLSME IRGSSRSYNA DLGGKFQF* |

A predicted signal peptide is highlighted.

The cp6731 nucleotide sequence <SEQ ID 34> is:

| | |
|---|---|
| 1 | ATGAAATCCT CTCTTCATTG GTTTTTAATC TCGTCATCTT TAGCACTTCC |
| 51 | CTTGTCACTA AATTTCTCTG CGTTTGCTGC TGTTGTTGAA ATCAATCTAG |
| 101 | GACCTACCAA TAGCTTCTCT GGACCAGGAA CCTACACTCC TCCAGCCCAA |
| 151 | ACAACAAATG CAGATGGAAC TATCTATAAT CTAACAGGGG ATGTCTCAAT |
| 201 | CACCAATGCA GGATCTCCGA CAGCTCTAAC CGCTTCCTGC TTTAAAGAAA |
| 251 | CTACTGGGAA TCTTTCTTTC CAAGGCCACG GCTACCAATT TCTCCTACAA |
| 301 | AATATCGATG CGGGAGCGAA CTGTACCTTT ACCAATACAG CTGCAAATAA |
| 351 | GCTTCTCTCC TTTTCAGGAT TCTCCTATTT GTCACTAATA CAAACCACGA |
| 401 | ATGCTACCAC AGGAACAGGA GCCATCAAGT CCACAGGAGC TTGTTCTATT |
| 451 | CAGTCGAACT ATAGTTGCTA CTTTGGCCAA AACTTTTCTA ATGACAATGG |
| 501 | AGGCGCCCTC CAAGGCAGCT CTATCAGTCT ATCGCTAAAC CCCAACCTAA |
| 551 | CGTTTGCCAA AAACAAAGCA ACGCAAAAAG GGGGTGCCCT CTATTCCACG |
| 601 | GGAGGGATTA CAATTAACAA TACGTTAAAC TCAGCATCAT TTTCTGAAAA |
| 651 | TACCGCGGCG AACAATGGCG GAGCCATTTA CACGGAAGCT AGCAGTTTTA |
| 701 | TTAGCAGCAA CAAAGCAATT AGCTTTATAA ACAATAGTGT GACCGCAACC |
| 751 | TCAGCTACAG GGGGAGCCAT TTACTGTAGT AGTACATCAG CCCCCAAACC |
| 801 | AGTCTTAACT CTATCAGACA ACGGGGAACT GAACTTTATA GGAAATACAG |
| 851 | CAATTACTAG TGGTGGGGCG ATTTATACTG ACAATCTAGT TCTTTCTTCT |
| 901 | GGAGGACCTA CGCTTTTTAA AAACAACTCT GCTATAGATA CTGCAGCTCC |
| 951 | CTTAGGAGGA GCAATTGCGA TTGCTGACTC TGGATCTTTG AGTCTTTCGG |
| 1001 | CTCTTGGTGG AGACATCACT TTTGAAGGAA ACACAGTAGT CAAAGGAGCT |
| 1051 | TCTTCGAGTC AGACCACTAC CAGAAATTCT ATTAACATCG GAAACACCAA |
| 1101 | TGCTAAGATT GTACAGCTGC GAGCCTCTCA AGGCAATACT ATCTACTTCT |
| 1151 | ATGATCCTAT AACAACTAGC ATCACTGCAG CTCTCTCAGA TGCTCTAAAC |
| 1201 | TTAAATGGTC CTGACCTTGC AGGGAATCCT GCATATCAAG GAACCATCGT |
| 1251 | ATTTTCTGGA GAGAAGCTCT CGGAAGCAGA AGCTGCAGAA GCTGATAATC |
| 1301 | TCAAATCTAC AATTCAGCAA CCTCTAACTC TTGCGGGAGG GCAACTCTCT |
| 1351 | CTTAAATCAG GAGTCACTCT AGTTGCTAAG TCCTTTTCGC AATCTCCGGG |
| 1401 | CTCTACCCTC CTCATGGATG CAGGGACCAC ATTAGAAACC GCTGATGGGA |
| 1451 | TCACTATCAA TAATCTTGTT CTCAATGTAG ATTCCTTAAA AGAGACCAAG |
| 1501 | AAGGCTACGC TAAAAGCAAC ACAAGCAAGT CAGACAGTCA CTTTATCTGG |
| 1551 | ATCGCTCTCT CTTGTAGATC CTTCTGGAAA TGTCTACGAA GATGTCTCTT |
| 1601 | GGAATAACCC TCAAGTCTTT TCTTGTCTCA CTCTTACTGC TGACGACCCC |
| 1651 | GCGAATATTC ACATCACAGA CTTAGCTGCT GATCCCCTAG AAAAAAATCC |
| 1701 | TATCCATTGG GGATACCAAG GGAATTGGGC ATTATCTTGG CAAGAGGATA |
| 1751 | CTGCGACTAA ATCCAAAGCA GCGACTCTTA CCTGGACAAA AACAGGATAC |
| 1801 | AATCCGAATC CTGAGCGTCG TGGAACCTTA GTTGCTAACA CGCTATGGGG |
| 1851 | ATCCTTTGTT GATGTGCGCT CCATACAACA GCTTGTAGCC ACTAAAGTAC |
| 1901 | GCCAATCTCA AGAAACTCGC GGCATCTGGT GTGAAGGGAT CTCGAACTTC |
| 1951 | TTCCATAAAG ATAGCACGAA GATAAATAAA GGTTTTCGCC ACATAAGTGC |
| 2001 | AGGTTATGTT GTAGGAGCGA CTACAACATT AGCTTCTGAT AATCTTATCA |
| 2051 | CTGCAGCCTT CTGCCAATTA TTCGGGAAAG ATAGAGATCA CTTTATAAAT |
| 2101 | AAAAATAGAG CTTCTGCCTA TGCAGCTTCT CTCCATCTCC AGCATCTAGC |
| 2151 | GACCTTGTCT TCTCCAAGCT TGTTACGCTA CCTTCCTGGA TCTGAAAGTG |
| 2201 | AGCAGCCTGT CCTCTTTGAT GCTCAGATCA GCTATATCTA TAGTAAAAAT |
| 2251 | ACTATGAAAA CCTATTACAC CCAAGCACCA AAGGGAGAGA GCTCGTGGTA |
| 2301 | TAATGACGGT TGCGCTCTGG AACTTGCGAG CTCCCTACCA CACACTGCTT |
| 2351 | TAAGCCATGA GGGTCTCTTC CACGCGTATT TTCCTTTCAT CAAAGTAGAA |
| 2401 | GCTTCGTACA TACACCAAGA TAGCTTCAAA GAACGTAATA CTACCTTGGT |
| 2451 | ACGATCTTTC GATAGCGGTG ATTTAATTAA CGTCTCTGTG CCTATTGGAA |
| 2501 | TTACCTTCGA GAGATTCTCG AGAAACGAGC GTGCGTCTTA CGAAGCTACT |
| 2551 | GTCATCTACG TTGCCGATGT CTATCGTAAG AATCCTGACT GCACGACAGC |
| 2601 | TCTCCTAATC AACAATACCT CGTGGAAAAC TACAGGAACG AATCTCTCAA |
| 2651 | GACAAGCTGG TATCGGAAGA GCAGGGATCT TTTATGCCTT CTCTCCAAAT |
| 2701 | CTTGAGGTCA CAAGTAACCT ATCTATGGAA ATTCGTGGAT CTTCACGCAG |
| 2751 | CTACAATGCA GATCTTGGAG GTAAGTTCCA GTTCTAA |

The PSORT algorithm predicts an outer membrane location (0.926).

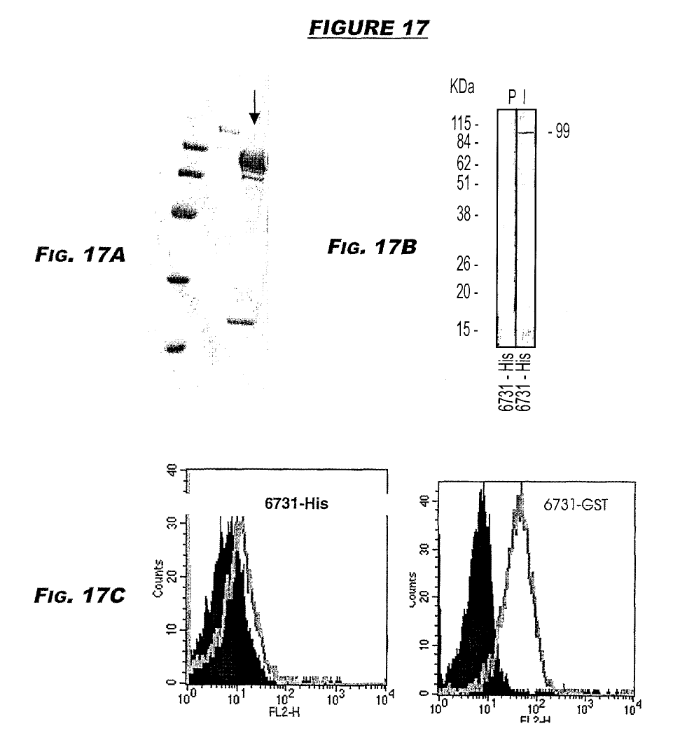

The protein was expressed in E. coli and purified as a his-tag product, as shown in FIG. 17A. A GST-fusion protein was also expressed. The recombinant proteins were used to immunize mice, whose sera were used in a Western blot (FIG. 17B; his-tag) and for FACS analysis (FIG. 17C; his-tag and GST-fusion).

The GST-fusion protein also showed good cross-reactivity with human sera, including sera from patients with pneumonitis. Less cross-reactivity was seen with the his-fusion.

These experiments show that cp6731 is a surface-exposed and immunoaccessible protein, and that it is a useful immunogen. These properties are not evident from the sequence alone.

### Example 18

The following C. pneumoniae protein (PID 4376737) was expressed <SEQ ID 35; cp6737>:

| | |
|---|---|
| 1 | MPLSFKSSSF CLLACLCSAD CAFAETRLGG NFVPPITNQG EEILLTSDFV |
| 51 | CSNFLGASFS SSFINSSSNL SLLGKGLSLT FTSCQAPTNS NYALLSAAET |
| 101 | LTFKNFSSIN FTGNQSTGLG GLIYGKDIVF QSIKDLIFTT NRVAYSPASV |
| 151 | TTSATPAITT VTTGASALQP TDSLTVENIS QSIKFFGNLA NFGSAISSSP |
| 201 | TAVVKFINNT ATMSFSHNFT SSGGGVIYGG SSLLFENNSG CIIFTANSCF |
| 251 | NSLKGVTPSS GTYALGSGGA ICIPTGTGEL KNNQGKCTFS YNGTPNDAGA |
| 301 | IYAETCNIVG NQGALLLDSN TAARNGGAIC AKVLNIQGRG PIEFSRNRAE |
| 401 | AGGEIVSLSA QGGSRLVFYD PITHSLPTTS PSNKDITINA NGASGSVVFT |
| 451 | SKGLSSTELL LPANTTTILL GTVKIASGEL KITDNAVVNV LGFATQGSGQ |
| 501 | LTLGSGGTLG LATPTGAPAA VDFTIGKLAF DPFSFLKRDF VSASVNAGTK |
| 551 | NVTLTGALVL DEHDVTDLYD MVSLQTPVAI PIAVFKGATV TKTGFPDGEI |
| 601 | ATPSHYGYQG KWSYTWSRPL LIPAPDGGFP GGPSPSANTL YAVWNSDTLV |
| 651 | RSTYILDPER YGEIVSNSLW ISFLGNQAFS DILQDVLLID HPGLSITAKA |
| 701 | LGAYVEHTPR QGHEGFSGRY GGYQAALSMN YTDHTTLGLS FGQLYGKTNA |
| 751 | NPYDSRCSEQ MYLLSFFGQF PIVTQKSEAL ISWKAAYGYS KNHLNTTYLR |
| 801 | PDKAPKSQGQ WHNNSYYVLI SAEHPFLNWC LLTRPLAQAW DLSGFISAEF |
| 851 | LGGWQSKFTE TGDLQRSFSR GKGYNVSLPI GCSSQWFTPF KKAPSTLTIK |
| 901 | LAYKPDIYRV NPHNIVTVVS NQESTSISGA NLRRHGLFVQ IHDVVDLTED |
| 951 | TQAFLNYTFD GKNGFTNHRV STGLKSTF* |

A predicted signal peptide is highlighted.

The cp6737 nucleotide sequence <SEQ ID 36> is:

| | |
|---|---|
| 1 | ATGCCTCTTT CTTTCAAATC TTCATCTTTT TGTCTACTTG CCTGTTTATG |
| 51 | TAGTGCAAGT TGCGCGTTTG CTGAGACTAG ACTCGGAGGG AACTTTGTTC |
| 101 | CTCCAATTAC GAATCAGGGT GAAGAGATCT TACTCACTTC AGATTTTGTT |
| 151 | TGTTCAAACT TCTTGGGGGC GAGTTTTTCA AGTTCCTTTA TCAATAGTTC |
| 201 | CAGCAATCTC TCCTTATTAG GGAAGGGCCT TTCCTTAACG TTTACCTCTT |
| 251 | GTCAAGCTCC TACAAATAGT AACTATGCGC TACTTTCTGC CGCAGAGACT |
| 301 | CTGACCTTCA AGAATTTTTC TTCTATAAAC TTTACAGGGA ACCAATCGAC |
| 351 | AGGACTTGGC GGCCTCATCT ACGGAAAAGA TATTGTTTTC CAATCTATCA |
| 401 | AAGATTTGAT CTTCACTACG AACCGTGTTG CCTATTCTCC AGCATCTGTA |
| 451 | ACTACGTCGG CAACTCCCGC AATCACTACA GTAACTACAG GAGCCTCTGC |
| 501 | TCTCCAACCT ACAGACTCAC TCACTGTCGA AAACATATCC CAATCGATCA |
| 551 | AGTTTTTTGG GAACCTTGCC AACTTCGGCT CTGCAATTAG CAGTTCTCCC |
| 601 | ACGGCAGTCG TTAAATTCAT CAATAACACC GCTACCATGA GCTTCTCCCA |
| 651 | TAACTTTACT TCGTCAGGAG GCGGCGTGAT TTATGGAGGA AGCTCTCTCC |
| 701 | TTTTTGAAAA CAATTCTGGA TGCATCATCT TCACCGCCAA CTCCTGTGTG |
| 751 | AACAGCTTAA AAGGCGTCAC CCCTTCATCA GGAACCTATG CTTTAGGAAG |
| 801 | TGGCGGAGCC ATCTGCATCC CTACGGGAAC TTTCGAATTA AAAAACAATC |
| 851 | AGGGGAAGTG CACCTTCTCT TATAATGGTA CACCAAATGA TGCGGGTGCG |
| 901 | ATCTACGCCG AAACCTGCAA CATCGTAGGG AACCAGGGTG CCTTGCTCCT |
| 951 | AGATAGCAAC ACTGCAGCGA GAAATGGCGG AGCCATCTGT GCTAAAGTGC |
| 1001 | TCAATATTCA AGGACGCGGT CCTATTGAAT TCTCTAGAAA CCGCGCGGAG |
| 1051 | AAGGGTGGAG CTATTTTCAT AGGCCCCTCT GTTGGAGACC CTGCGAAGCA |
| 1101 | AACATCGACA CTTACGATTT TGGCTTCCGA AGGTGATATT GCGTTCCAAG |
| 1151 | GAAACATGCT CAATACAAAA CCTGGAATCC GCAATGCCAT CACTGTAGAA |
| 1201 | GCAGGGGGAG AGATTGTGTC TCTATCTGCA CAAGGAGGCT CACGTCTTGT |
| 1251 | ATTTTATGAT CCCATTACAC ATAGCCTCCC AACCACAAGT CCGTCTAATA |
| 1301 | AAGACATTAC AATCAACGCT AATGGCGCTT CAGGATCTGT AGTCTTTACA |
| 1351 | AGTAAGGGAC TCTCCTCTAC AGAACTCCTG TTGCCTGCCA ACACGACAAC |
| 1401 | TATACTTCTA GGAACAGTCA AGATCGCTAG TGGAGAACTG AAGATTACTG |
| 1451 | ACAATGCGGT TGTCAATGTT CTTGGCTTCG CTACTCAGGG CTCAGGTCAG |
| 1501 | CTTACCCTGG GCTCTGGAGG AACCTTAGGG CTGGCAACAC CCACGGGAGC |
| 1551 | ACCTGCCGCT GTAGACTTTA CGATTGGAAA GTTAGCATTC GATCCTTTTT |
| 1601 | CCTTCCTAAA AAGAGATTTT GTTTCAGCAT CAGTAAATGC AGGCACAAAA |
| 1651 | AACGTCACTT TAACAGGAGC TCTGGTTCTT GATGAACATG ACGTTACAGA |
| 1701 | TCTTTATGAT ATGGTGTCAT TACAAACTCC AGTAGCAATT CCTATCGCTG |
| 1751 | TTTTCAAAGG AGCAACCGTT ACTAAGACAG GATTTCCTGA TGGGGAGATT |
| 1801 | GCGACTCCAA GCCACTACGG CTACCAAGGA AAGTGGTCCT ACACATGGTC |
| 1851 | CCGTCCCCTG TTAATTCCAG CTCCTGATGG AGGATTTCCT GGAGGTCCCT |
| 1901 | CTCCTAGCGC AAATACTCTC TATGCTGTAT GGAATTCAGA CACTCTCGTG |
| 1951 | CGTTCTACCT ATATCTTAGA TCCCGAGCGT TACGGAGAAA TTGTCAGCAA |
| 2001 | CAGCTTATGG ATTTCCTTCT TAGGAAATCA GGCATTCTCT GATATTCTCC |
| 2051 | AAGATGTTCT TTTGATAGAT CATCCCGGGT TGTCCATAAC CGCGAAAGCT |
| 2101 | TTAGGAGCCT ATGTCGAACA CACACCAAGA CAAGGACATG AGGGCTTTTC |
| 2151 | AGGTCGCTAT GGAGGCTACC AAGCTGCGCT ATCTATGAAC TACACGGACC |
| 2201 | ACACTACGTT AGGACTTTCT TTCGGGCAGC TTTATGGAAA AACTAACGCC |
| 2251 | AACCCCTACG ATTCACGTTG CTCAGAACAA ATGTATTTAC TCTCGTTCTT |
| 2301 | TGGTCAATTC CCTATCGTGA CTCAAAAGAG CGAGGCCTTA ATTTCCTGGA |
| 2351 | AAGCAGCTTA TGGTTATTCC AAAAATCACC TAAATACCAC CTACCTCAGA |
| 2401 | CCTGACAAAG CTCCAAAATC TCAAGGGCAA TGGCATAACA ATAGTTACTA |
| 2451 | TGTTCTTATT TCTGCAGAAC ATCCTTTCCT AAACTGGTGT CTTCTTACAA |
| 2501 | GACCTCTGGC TCAAGCTTGG GATCTTTCAG GTTTTATTTC CGCAGAATTC |
| 2551 | CTAGGTGGTT GGCAAAGTAA GTTCACAGAA ACTGGAGATC TGCAACGTAG |
| 2601 | CTTTAGTAGA GGTAAAGGGT ACAATGTTTC CCTACCGATA GGATGTTCTT |
| 2651 | CTCAATGGTT CACACCATTT AAGAAGGCTC CTTCTACACT GACCATCAAA |
| 2701 | CTTGCCTACA AGCCTGATAT CTATCGTGTC AACCCTCACA ATATTGTGAC |
| 2751 | TGTCGTCTCA AACCAAGAGA GCACTTCGAT CTCAGGAGCA AATCTACGCC |
| 2801 | GCCACGGTTT GTTTGTACAA ATCCATGATG TAGTAGATCT CACCGAGGAC |
| 2851 | ACTCAGGCCT TTCTAAACTA TACCTTTGAC GGGAAAAATG GATTTACAAA |
| 2901 | CCACCGAGTG TCTACAGGAC TAAAATCCAC ATTTTAA |

The PSORT algorithm predicts an outer membrane location (0.940).

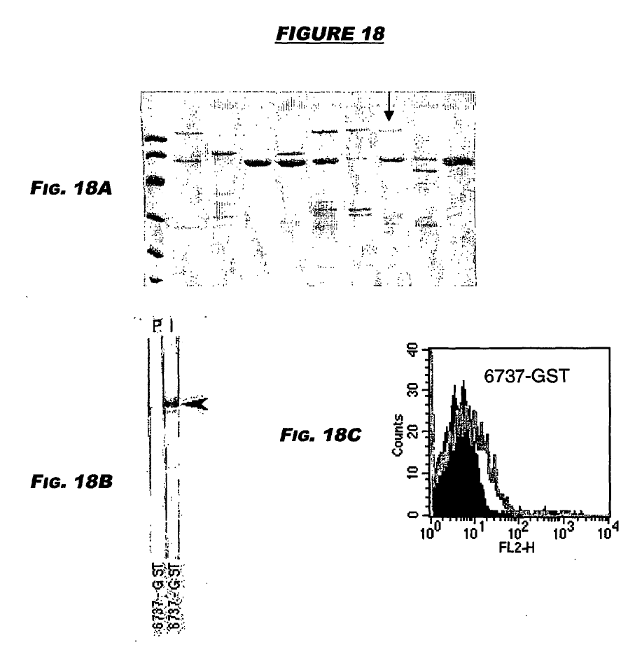

The protein was expressed in E. coli and purified as a GST-fusion product, as shown in FIG. 18A. The recombinant protein was used to immunize mice, whose sera were used in an immunoblot analysis blot (FIG. 18B) and for FACS analysis (FIG. 18C). A his-tagged protein was also expressed.

The cp6737 protein was also identified in the 2D-PAGE experiment (Cpn0454) and showed good cross-reactivity with human sera, including sera from patients with pneumonitis.

These experiments show that cp6737 is a surface-exposed and immunoaccessible protein, and that it is a useful immunogen. These properties are not evident from the sequence alone.

### Example 19

The following C. pneumoniae protein (PID 4377090) was expressed <SEQ ID 37; cp7090>:

| | |
|---|---|
| 1 | MNIHSLWKLC TLLALLALPA CSLSPNYGWE DSCNTCHHTR RKKPSSFGFV |
| 51 | PLYTEEDFNP NFTFGEYDSK EEKQYKSSQV AAFRNITFAT DSYTIKGEEN |
| 101 | LAILTNLVHY MKKNPKATLY IEGHTDERGA ASYNLALGAR RANAIKEHLR |
| 151 | KQGISADRLS TISYGKEHPL NSGHNELAWQ QNRRTEFKIH AR* |

A predicted signal peptide is highlighted.

The cp7090 nucleotide sequence <SEQ ID 38> is:

| | |
|---|---|
| 1 | ATGAATATAC ATTCCCTATG GAAACTTTGT ACTTTATTGG CTTTACTTGC |
| 51 | ATTGCCAGCA TGTAGCCTTT CCCCTAATTA TGGCTGGGAG GATTCCTGTA |
| 101 | ATACATGCCA TCATACAAGA CGAAAAAAGC CTTCTTCTTT TGGCTTTGTT |
| 151 | CCTCTCTATA CCGAAGAGGA CTTTAACCCT AATTTTACCT TCGGTGAGTA |
| 201 | TGATTCCAAA GAAGAAAAAC AATACAAGTC AAGCCAAGTT GCAGCATTTC |
| 251 | GTAATATCAC CTTTGCTACA GACAGCTATA CAATTAAAGG TGAAGAGAAC |
| 301 | CTTGCGATTC TCACGAACTT GGTTCACTAC ATGAAGAAAA ACCCGAAAGC |
| 351 | TACACTGTAC ATTGAAGGGC ATACTGACGA GCGTGGAGCT GCATCCTATA |
| 401 | ACCTTGCTTT AGGAGCACGA CGAGCCAATG CGATTAAAGA GCATCTCCGA |
| 451 | AAGCAGGGAA TCTCTGCAGA TCGTCTATCT ACTATTTCCT ACGGAAAAGA |
| 501 | ACATCCTTTA AATTCGGGAC ACAACGAACT AGCATGGCAA CAAAATCGCC |
| 551 | GTACAGAGTT TAAGATTCAT GCACGCTAA |

The PSORT algorithm predicts an outer membrane location (0.790).

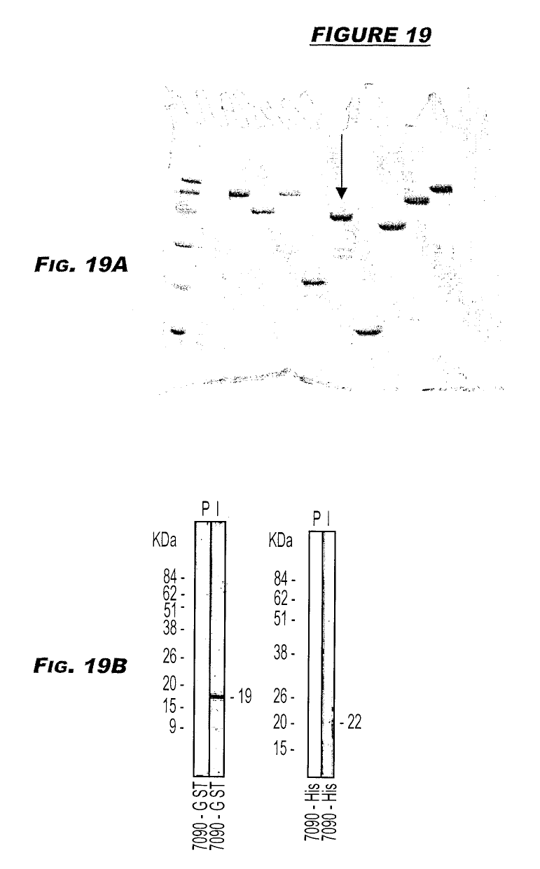

The protein was expressed in E. coli and purified as a GST-fusion product, as shown in FIG. 19A. A his-tagged protein was also expressed. The recombinant proteins were used to immunize mice, whose sera were used in a Western blot (FIG. 19B) and for FACS analysis.

These experiments show that cp7090 is useful immunogen. These properties are not evident from the sequence alone.

### Example 20

The following C. pneumoniae protein (PID 4377091) was expressed <SEQ ID 39; cp7091>:

| | |
|---|---|
| 1 | MLRQLCFQVF FFCFASLVYA EELEVVVRSE HITLPIEVSC QTDTKDPKIQ |
| 51 | KYLSSLTEIF CKDIALGDCL QPTAASKESS SPLAISLRLH VPQLSVVLLQ |
| 101 | SSKTPQTLCS FTISQNLSVD RQKIHHAADT VHYALTGIPG ISAGKIVFAL |
| 151 | SSLGKDQKLK QGELWTTDYD GKNLAPLTTE CSLSITPKWV GVGSNFPYLY |
| 201 | VSYKYGVPKI FLGSLENTEG KKVLPLKGNQ LMPTFSPRKK LLAFVADTYG |
| 251 | NPDLFIQPFS LTSGPMGRPR RLLNENFGTQ GNPSFNPEGS QLVFISNKDG |
| 301 | RPRLYIMSLD PEPQAPRLLT KKYRNSSCPA WSPDGKKIAF CSVIKGVRQI |
| 351 | CIYDLSSGED YQLTTSPTNK ESPSWAIDSR HLVFSAGNAE ESELYLISLV |
| 401 | TKKTNKIAIG VGEKRFPSWG AFPQQPIKRT L* |

A predicted signal peptide is highlighted.

The cp7091 nucleotide sequence <SEQ ID 40> is:

| | |
|---|---|
| 1 | ATGTTACGGC AACTATGCTT CCAAGTTTTT TTCTTTTGCT TCGCATCGCT |
| 51 | AGTCTATGCT GAAGAATTAG AAGTTGTTGT CCGTTCCGAA CATATCACGC |
| 101 | TCCCTATTGA GGTCTCTTGC CAGACCGATA CGAAAGATCC AAAAATACAG |
| 151 | AAATACCTCA GCTCGCTAAC GGAGATATTT TGCAAGGACA TTGCCCTAGG |
| 201 | AGATTGTCTA CAACCCACAG CGGCTTCTAA AGAATCGTCA TCTCCTTTAG |
| 251 | CAATATCTTT ACGGTTGCAT GTACCTCAGC TATCTGTAGT GCTTTTACAG |
| 301 | TCTTCAAAAA CTCCTCAAAC CTTATGTTCT TTTACTATTT CTCAAAATCT |
| 351 | TTCTGTAGAT CGTCAAAAAA TCCATCACGC TGCTGATACA GTTCATTACG |
| 401 | CCCTCACAGG GATTCCTGGA ATCAGTGCTG GGAAAATTGT TTTTGCTCTA |
| 451 | AGTTCTTTAG GAAAAGATCA AAAGCTCAAG CAAGGAGAAT TATGGACTAC |
| 501 | AGATTACGAT GGGAAAAACC TCGCCCCTTT AACCACAGAA TGTTCGCTCT |
| 551 | CTATAACTCC AAAATGGGTG GGTGTGGGAT CAAATTTTCC CTATCTCTAT |
| 601 | GTTTCGTATA AGTATGGTGT GCCTAAAATT TTTCTTGGTT CCCTAGAGAA |
| 651 | CACTGAAGGT AAAAAAGTCC TTCCGTTAAA AGGCAACCAA CTCATGCCTA |
| 701 | CGTTTTCTCC AAGAAAAAAG CTTTTAGCTT TCGTTGCTGA TACGTATGGA |
| 751 | AATCCTGATT TATTTATTCA ACCGTTCTCA CTAACTTCAG GACCTATGGG |
| 801 | TCGCCCACGT CGCCTCCTTA ATGAGAATTT CGGGACTCAA GGGAATCCCT |
| 851 | CCTTCAACCC TGAAGGATCC CAGCTTGTCT TTATATCGAA CAAAGACGGC |
| 901 | CGTCCGCGTC TTTATATTAT GTCCCTCGAT CCTGAACCCC AAGCACCTCG |
| 951 | CTTGCTGACA AAAAAATACA GAAATAGCAG TTGCCCTGCA TGGTCTCCAG |
| 1001 | ATGGTAAAAA AATAGCCTTC TGCTCTGTAA TTAAAGGGGT GCGACAAATT |
| 1051 | TGTATTTACG ATCTCTCCTC TGGAGAGGAT TACCAACTCA CTACGTCTCC |
| 1101 | CACAAATAAA GAGAGTCCTT CTTGGGCTAT AGACAGCCGT CATCTTGTCT |
| 1151 | TTAGTGCGGG GAATGCTGAA GAATCAGAGT TATATTTAAT CAGTCTAGTC |
| 1201 | ACCAAAAAAA CTAACAAAAT TGCTATAGGA GTAGGAGAAA AACGGTTCCC |
| 1251 | CTCCTGGGGT GCTTTCCCTC AGCAACCGAT AAAGAGAACA CTATGA |

The PSORT algorithm predicts an inner membrane location (0.109).

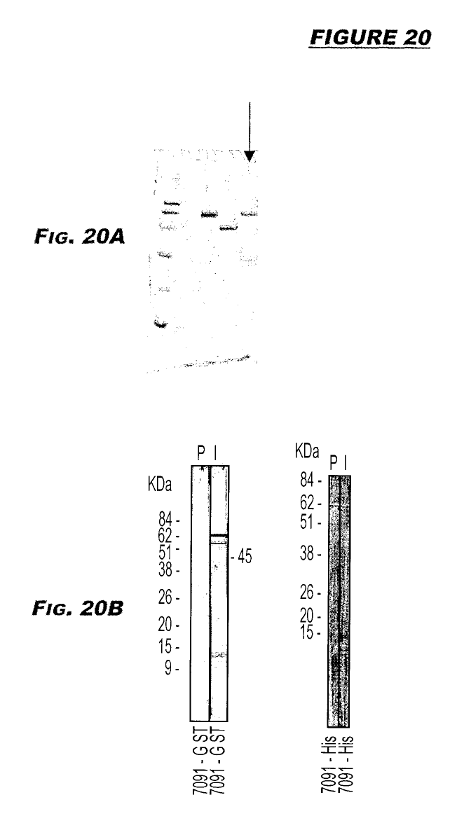

The protein was expressed in E. coli and purified as a GST-fusion product, as shown in FIG. 20A. A his-tagged protein was also expressed. The recombinant proteins were used to immunize mice, whose sera were used in a Western blot (FIG. 20B) and for FACS analysis.

These experiments show that cp7091 is a useful immunogen. These properties are not evident from the sequence alone.

### Example 21

The following C. pneumoniae protein (PID 4376260) was expressed <SEQ ID 41; cp6260>:

| | |
|---|---|
| 1 | MRFSLCGFPL VFSFTLLSVF DTSLSATTIS LTPEDSFHGD SQNAERSYNV |
| 51 | QAGDVYSLTG DVSISNVDNS ALNKACFNVT SGSVTFAGNH HGLYFNNISS |
| 101 | GTTKEGAVLC CQDPQATARF SGFSTLSFIQ SPGDIKEQGC LYSKNALMLL |
| 151 | NNYVVRFEQN QSKTKGGAIS GANVTIVGNY DSVSFYQNAA TFGGAIHSSG |
| 201 | PLQIAVNQAE IRFAQNTAKN GSGGALYSDG DIDIDQNAYV LFRENEALTT |
| 251 | AIGKGGAVCC LPTSGSSTPV PIVTFSDNKQ LVFERNHSIM GGGAIYARKL |
| 301 | SISSGGPTLF INNISYANSQ NLGGAIAIDT GGEISLSAEK GTITFQGNRT |
| 351 | SLPFLNGIHL LQNAKFLKLQ ARNGYSIEFY DPITSEADGS TQLNINGDPK |
| 401 | NKEYTGTILF SGEKSLANDP RDFKSTIPQN VNLSAGYLVI KEGAEVTVSK |
| 451 | FTQSPGSHLV LDLGTKLIAS KEDIAITGLA IDIDSLSSSS TAAVIKANTA |
| 501 | NKQISVTDSI ELISPTGNAY EDLRMRNSQT FPLLSLEPGA GGSVTVTAGD |
| 551 | FLPVSPHYGF QGNWKLAWTG TGNKVGEFFW DKINYKPRPE KEGNLVPNIL |
| 601 | WGNAVDVRSL MQVQETHASS LQTDRGLWID GIGNFFHVSA SEDNIRYRHN |
| 651 | SGGYVLSVNN EITPKHYTSM AFSQLFSRDK DYAVSNNEYR MYLGSYLYQY |
| 701 | TTSLGNIFRY ASRNPNVNVG ILSRRFLQNP LMIFHFLCAY GHATNDMKTD |
| 751 | YANFPMVKNS WRNNCWAIEC GGSMPLLVFE NGRLFQGAIP FMKLQLVYAY |
| 801 | QGDFKETTAD GRRFSNGSLT SISVPLGIRF EKLALSQDVL YDFSFSYIPD |
| 851 | IFRKDPSCEA ALVISGDSWL VPAAHVSRHA FVGSGTGRYH FNDYTELLCR |
| 901 | GSIECRPHAR NYNINCGSKF RF* |

A predicted signal peptide is highlighted.

The cp6260 nucleotide sequence <SEQ ID 42> is:

| | |
|---|---|
| 1 | ATGCGATTTT CGCTCTGCGG ATTTCCTCTA GTTTTTTCTT TTACATTGCT |
| 51 | CTCAGTCTTC GACACTTCTT TGAGTGCTAC TACGATTTCT TTAACCCCAG |
| 101 | AAGATAGTTT TCATGGAGAT AGTCAGAATG CAGAACGTTC TTATAATGTT |
| 151 | CAAGCTGGGG ATGTCTATAG CCTTACTGGT GATGTCTCAA TATCTAACGT |
| 201 | CGATAACTCT GCATTAAATA AAGCCTGCTT CAATGTGACC TCAGGAAGTG |
| 251 | TGACGTTCGC AGGAAATCAT CATGGGTTAT ATTTTAATAA TATTTCCTCA |
| 301 | GGAACTACAA AGGAAGGGGC TGTACTTTGT TGCCAAGATC CTCAAGCAAC |
| 351 | GGCACGTTTT TCTGGGTTCT CCACGCTCTC TTTTATTCAG AGCCCCGGAG |
| 401 | ATATTAAAGA ACAGGGATGT CTCTATTCAA AAAATGCACT TATGCTCTTA |
| 451 | AACAATTATG TAGTGCGTTT TGAACAAAAC CAAAGTAAGA CTAAAGGCGG |
| 501 | AGCTATTAGT GGGGCGAATG TTACTATAGT AGGCAACTAC GATTCCGTCT |
| 551 | CTTTCTATCA GAATGCAGCC ACTTTTGGAG GTGCTATCCA TTCTTCAGGT |
| 601 | CCCCTACAGA TTGCAGTAAA TCAGGCAGAG ATAAGATTTG CACAAAATAC |
| 651 | TGCCAAGAAT GGTTCTGGAG GGGCTTTGTA CTCCGATGGT GATATTGATA |
| 701 | TTGATCAGAA TGCTTATGTT CTATTTCGAG AAAATGAGGC ATTGACTACT |
| 751 | GCTATAGGTA AGGGAGGGGC TGTCTGTTGT CTTCCCACTT CAGGAAGTAG |
| 801 | TACTCCAGTT CCTATTGTGA CTTTCTCTGA CAATAAACAG TTAGTCTTTG |
| 851 | AAAGAAACCA TTCCATAATG GGTGGCGGAG CCATTTATGC TAGGAAACTT |
| 901 | AGCATCTCTT CAGGAGGTCC TACTCTATTT ATCAATAATA TATCATATGC |
| 951 | AAATTCGCAA AATTTAGGTG GAGCTATTGC CATTGATACT GGAGGGGAGA |
| 1001 | TCAGTTTATC AGCAGAGAAA GGAACAATTA CATTCCAAGG AAACCGGACG |
| 1051 | AGCTTACCGT TTTTGAATGG CATCCATCTT TTACAAAATG CTAAATTCCT |
| 1101 | GAAATTACAG GCGAGAAATG GATACTCTAT AGAATTTTAT GATCCTATTA |
| 1151 | CTTCTGAAGC AGATGGGTCT ACCCAATTGA ATATCAACGG AGATCCTAAA |
| 1201 | AATAAAGAGT ACACAGGGAC CATACTCTTT TCTGGAGAAA AGAGTCTAGC |
| 1251 | AAACGATCCT AGGGATTTTA AATCTACAAT CCCTCAGAAC GTCAACCTGT |
| 1301 | CTGCAGGATA CTTAGTTATT AAAGAGGGGG CCGAAGTCAC AGTTTCAAAA |
| 1351 | TTCACGCAGT CTCCAGGATC GCATTTAGTT TTAGATTTAG GAACCAAACT |
| 1401 | GATAGCCTCT AAGGAAGACA TTGCCATCAC AGGCCTCGCG ATAGATATAG |
| 1451 | ATAGCTTAAG CTCATCCTCA ACAGCAGCTG TTATTAAAGC AAACACCGCA |
| 1501 | AATAAACAGA TATCCGTGAC GGACTCTATA GAACTTATCT CGCCTACTGG |
| 1551 | CAATGCCTAT GAAGATCTCA GAATGAGAAA TTCACAGACG TTCCCTCTGC |
| 1601 | TCTCTTTAGA GCCTGGAGCC GGGGGTAGTG TGACTGTAAC TGCTGGAGAT |
| 1651 | TTCCTACCGG TAAGTCCCCA TTATGGTTTT CAAGGCAATT GGAAATTAGC |
| 1701 | TTGGACAGGA ACTGGAAACA AAGTTGGAGA ATTCTTCTGG GATAAAATAA |
| 1751 | ATTATAAGCC TAGACCTGAA AAAGAAGGAA ATTTAGTTCC TAATATCTTG |
| 1801 | TGGGGGAATG CTGTAGATGT CAGATCCTTA ATGCAGGTTC AAGAGACCCA |
| 1851 | TGCATCGAGC TTACAGACAG ATCGAGGGCT GTGGATCGAT GGAATTGGGA |
| 1901 | ATTTCTTCCA TGTATCTGCC TCCGAAGACA ATATAAGGTA CCGTCATAAC |
| 1951 | AGCGGTGGAT ATGTTCTATC TGTAAATAAT GAGATCACAC CTAAGCACTA |
| 2001 | TACTTCGATG GCATTTTCCC AACTCTTTAG TAGAGACAAG GACTATGCGG |
| 2051 | TTTCCAACAA CGAATACAGA ATGTATTTAG GATCGTATCT CTATCAATAT |
| 2101 | ACAACCTCCC TAGGGAATAT TTTCCGTTAT GCTTCGCGTA ACCCTAATGT |
| 2151 | AAACGTCGGG ATTCTCTCAA GAAGGTTTCT TCAAAATCCT CTTATGATTT |
| 2201 | TTCATTTTTT GTGTGCTTAT GGTCATGCCA CCAATGATAT GAAAACAGAC |
| 2251 | TACGCAAATT TCCCTATGGT GAAAAACAGC TGGAGAAACA ATTGTTGGGC |
| 2301 | TATAGAGTGC GGAGGGAGCA TGCCTCTATT GGTATTTGAG AACGGAAGAC |
| 2351 | TTTTCCAAGG TGCCATCCCA TTTATGAAAC TACAATTAGT TTATGCTTAT |
| 2401 | CAGGGAGATT TCAAAGAGAC GACTGCAGAT GGCCGTAGAT TTAGTAATGG |
| 2451 | GAGTTTAACA TCGATTTCTG TACCTCTAGG CATACGCTTT GAGAAGCTGG |
| 2501 | CACTTTCTCA GGATGTACTC TATGACTTTA GTTTCTCCTA TATTCCTGAT |
| 2551 | ATTTTCCGTA AGGATCCCTC ATGTGAAGCT GCTCTGGTGA TTAGCGGAGA |
| 2601 | CTCCTGGCTT GTTCCGGCAG CACACGTATC AAGACATGCT TTTGTAGGGA |
| 2651 | GTGGAACGGG TCGGTATCAC TTTAACGACT ATACTGAGCT CTTATGTCGA |
| 2701 | GGAAGTATAG AATGCCGCCC CCATGCTAGG AATTATAATA TAAACTGTGG |
| 2751 | AAGCAAATTT CGTTTTTAG |

The PSORT algorithm predicts an outer membrane location (0.921).

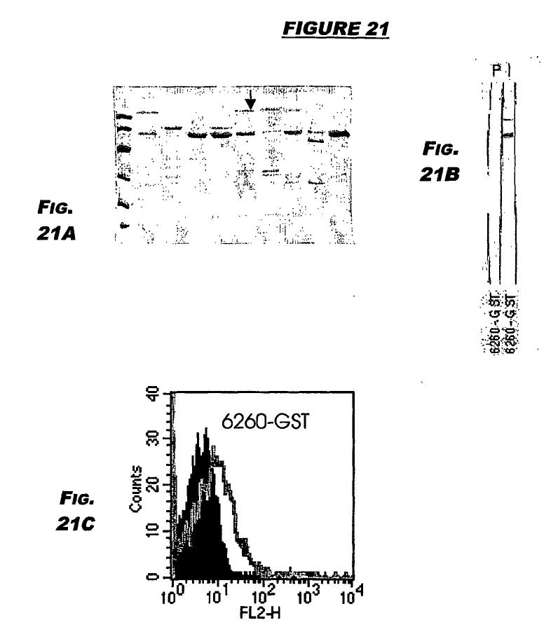

The protein was expressed in E. coli and purified both as a his-tag and GST-fusion product. The GST-fusion is shown in FIG. 21A. This recombinant protein was used to immunize mice, whose sera were used in a Western blot (FIG. 21B) and for FACS analysis (FIG. 21C).

This protein also showed good cross-reactivity with human sera, including sera from patients with pneumonitis.

These experiments show that cp6260 is a surface-exposed and immunoaccessible protein, and that it is a useful immunogen. These properties are not evident from the sequence alone.

### Example 22

The following C. pneumoniae protein (PID 4376456) was expressed <SEQ ID 43; cp6456>:

| | |
|---|---|
| 1 | MSSPVNNTPS APNIPIPAPT TPGIPTTKPR SSFIEKVIIV AKYILFAIAA |
| 51 | TSGALGTILG LSGALTPGIG IALLVIFFVS MVLLGLILKD SISGGEERRL |
| 101 | REEVSRFTSE NQRLTVITTT LETEVKDLKA AKDQLTLEIE AFRNENGNLK |
| 151 | TTAEDLEEQV SKLSEQLEAL ERINQLIQAN AGDAQEISSE LKKLISGWDS |
| 201 | KVVEQINTSI QALKVLLGQE WVQEAQTHVK AMQEQIQALQ AEILGMHNQS |
| 251 | TALQKSVENL LVQDQALTRV VGELLESENK LSQACSALRQ EIEKLAQHET |
| 301 | SLQQRIDAML AQEQNLAEQV TALEKMKQEA QKAESEFIAC VRDRTFGRRE |
| 351 | TPPPTTPVVE GDESQEEDEG GTPPVSQPSS PVDRATGDGQ * |

The cp6456 nucleotide sequence <SEQ ID 44> is:

| | |
|---|---|
| 1 | ATGTCATCTC CTGTAAATAA CACACCCTCA GCACCAAACA TTCCAATACC |
| 51 | AGCGCCCACG ACTCCAGGTA TTCCTACAAC AAAACCTCGT TCTAGTTTCA |
| 101 | TTGAAAAGGT TATCATTGTA GCTAAGTACA TACTATTTGC AATTGCAGCC |
| 151 | ACATCAGGAG CACTCGGAAC AATTCTAGGT CTATCTGGAG CGCTAACCCC |
| 201 | AGGAATAGGT ATTGCCCTTC TTGTTATCTT CTTTGTTTCT ATGGTGCTTT |
| 251 | TAGGTTTAAT CCTTAAAGAT TCTATAAGTG GAGGAGAAGA ACGCAGGCTC |
| 301 | AGAGAAGAGG TCTCTCGATT TACAAGTGAG AATCAACGGT TGACAGTCAT |
| 351 | AACCACAACA CTTGAGACTG AAGTAAAGGA TTTAAAAGCA GCTAAAGATC |
| 401 | AACTTACACT TGAAATCGAA GCATTTAGAA ATGAAAACGG TAATTTAAAA |
| 451 | ACAACTGCTG AGGACTTAGA AGAGCAGGTT TCTAAACTTA GCGAACAATT |
| 501 | AGAAGCACTA GAGCGAATTA ATCAACTTAT CCAAGCAAAC GCTGGAGATG |
| 551 | CTCAAGAAAT TTCGTCTGAA CTAAAGAAAT TAATAAGCGG TTGGGATTCC |
| 601 | AAAGTTGTTG AACAGATAAA TACTTCTATT CAAGCATTGA AAGTGTTATT |
| 651 | GGGTCAAGAG TGGGTGCAAG AGGCTCAAAC ACACGTTAAA GCAATGCAAG |
| 701 | AGCAAATTCA AGCATTGCAA GCTGAAATTC TAGGAATGCA CAATCAATCT |
| 751 | ACAGCATTGC AAAAGTCAGT TGAGAATCTA TTAGTACAAG ATCAAGCTCT |
| 801 | AACAAGAGTA GTAGGTGAGT TGTTAGAGTC TGAGAACAAG CTAAGCCAAG |
| 851 | CTTGTTCTGC GCTACGTCAA GAAATAGAAA AGTTGGCCCA ACATGAAACA |
| 901 | TCTTTGCAAC AACGTATTGA TGCGATGCTA GCCCAAGAGC AAAATTTGGC |
| 951 | AGAGCAGGTC ACAGCCCTTG AAAAAATGAA ACAAGAAGCT CAGAAGGCTG |
| 1001 | AGTCCGAGTT CATTGCTTGT GTACGTGATC GAACTTTCGG ACGTCGTGAA |
| 1051 | ACACCTCCAC CAACAACACC TGTAGTTGAA GGTGATGAAA GTCAAGAAGA |
| 1101 | AGACGAAGGA GGTACTCCCC CAGTATCACA ACCATCTTCA CCCGTAGATA |
| 1151 | GAGCAACAGG AGATGGTCAG TAA |

The PSORT algorithm predicts inner membrane (0.127).

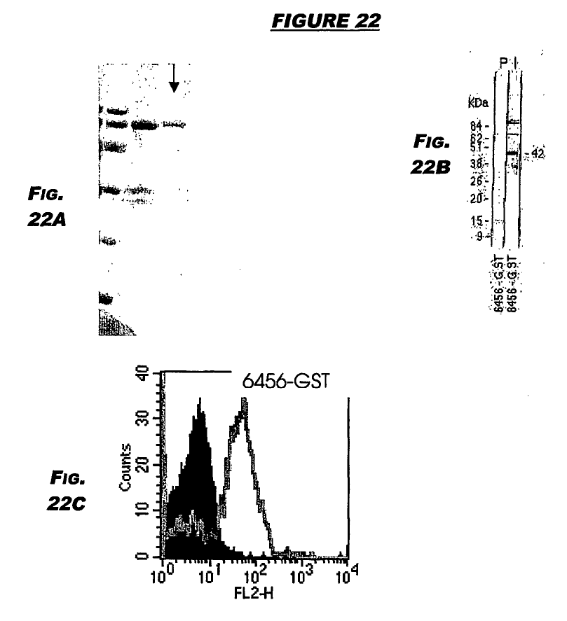

The protein was expressed in E. coli and purified as a GST-fusion product, as shown in FIG. 22A. The recombinant protein was used to immunize mice, whose sera were used in a Western blot (FIG. 22B) and for FACS analysis (FIG. 22C). A his-tag protein was also expressed.

These experiments show that cp6456 is a surface-exposed and immunoaccessible protein, and that it is a useful immunogen. These properties are not evident from the sequence alone.

### Example 23

The following C. pneumoniae protein (PID 4376729) was expressed <SEQ ID 45; cp6729>:

| | |
|---|---|
| 1 | MKIPLHKLLI SSTLVTPILL SIATYGADAS LSPTDSFDGA GGSTFTPKST |
| 51 | ADANGTNYVL SGNVYINDAG KGTALTGCCF TETTGDLTFT GKGYSFSFNT |
| 101 | VDAGSNAGAA ASTTADKALT FTGFSNLSFI AAPGTTVASG KSTLSSAGAL |
| 151 | NLTDNGTILF SQNVSNEANN NGGAITTKTL STSGNTSSIT FTSNSAKKLG |
| 201 | GAIYSSAAAS ISGNTGQLVF MNNKGETGGG ALGFEASSSI TQNSSLFFSG |
| 251 | NTATDAAGKG GAIYCEKTGE TPTLTISGNK SLTFAENSSV TQGGAICAHG |
| 301 | LDLSAAGPTL FSNNRCGNTA AGKGGAIAIA DSGSLSLSAN QGDITFLGNT |
| 351 | LTSTSAPTST RNAIYLGSSA KITNLRAAQG QSIYFYDPIA SNTTGASDVL |
| 401 | TINQPDSNSP LDYSGTIVFS GEKLSADEAK AADNFTSILK QPLALASGTL |
| 451 | ALKGNVELDV NGFTQTEGST LLMQPGTKLK ADTEAISLTK LVVDLSALEG |
| 501 | NKSVSIETAG ANKTITLTSP LVFQDSSGNF YESHTINQAF TQPLVVFTAA |
| 551 | TAASDIYIDA LLTSPVQTPE PHYGYQGHWE ATWADTSTAK SGTMTWVTTG |
| 601 | YNPNPERRAS VVPDSLWASF TDIRTLQQIM TSQANSIYQQ RGLWASGTAN |
| 651 | FFHKDKSGTN QAFRHKSYGY IVGGSAEDFS ENIFSVAFCQ LFGKDKDLFI |
| 701 | VENTSHNYLA SLYLQHRAFL GGLPMPSFGS ITDMLKDIPL ILNAQLSYSY |
| 751 | TKNDMDTRYT SYPEAQGSWT NNSGALELGG SLALYLPKEA PFFQGYFPFL |
| 801 | KFQAVYSRQQ NFKESGAEAR AFDDGDLVNC SIPVGIRLEK ISEDEKNNFE |
| 851 | ISLAYIGDVY RKNPRSRTSL MVSGASWTSL CKNLARQAFL ASAGSHLTLS |
| 901 | PHVELSGEAA YELRGSAHIY NVDCGLRYSF * |

A predicted signal peptide is highlighted.

The cp6729 nucleotide sequence <SEQ ID 46> is:

| | |
|---|---|
| 1 | ATGAAAATAC CCTTGCACAA ACTCCTGATC TCTTCGACTC TTGTCACTCC |
| 51 | CATTCTATTG AGCATTGCAA CTTACGGAGC AGATGCTTCT TTATCCCCTA |
| 101 | CAGATAGCTT TGATGGAGCG GGCGGCTCTA CATTTACTCC AAAATCTACA |
| 151 | GCAGATGCCA ATGGAACGAA CTATGTCTTA TCAGGAAATG TCTATATAAA |
| 201 | CGATGCTGGG AAAGGCACAG CATTAACAGG CTGCTGCTTT ACAGAAACTA |
| 251 | CGGGTGATCT GACATTTACT GGAAAGGGAT ACTCATTTTC ATTCAACACG |
| 301 | GTAGATGCGG GTTCGAATGC AGGAGCTGCG GCAAGCACAA CTGCTGATAA |
| 351 | AGCCCTAACA TTCACAGGAT TTTCTAACCT TTCCTTCATT GCAGCTCCTG |
| 401 | GAACTACAGT TGCTTCAGGA AAAAGTACTT TAAGTTCTGC AGGAGCCTTA |
| 451 | AATCTTACCG ATAATGGAAC GATTCTCTTT AGCCAAAACG TCTCCAATGA |
| 501 | AGCTAATAAC AATGGCGGAG CGATCACCAC AAAAACTCTT TCTATTTCTG |
| 551 | GGAATACCTC TTCTATAACC TTCACTAGTA ATAGCGCAAA AAAATTAGGT |
| 601 | GGAGCGATCT ATAGCTCTGC GGCTGCAAGT ATTTCAGGAA ACACCGGCCA |
| 651 | GTTAGTCTTT ATGAATAATA AAGGAGAAAC TGGGGGTGGG GCTCTGGGCT |
| 701 | TTGAAGCCAG CTCCTCGATT ACTCAAAATA GCTCCCTTTT CTTCTCTGGA |
| 751 | AACACTGCAA CAGATGCTGC AGGCAAGGGC GGGGCCATTT ATTGTGAAAA |
| 801 | AACAGGAGAG ACTCCTACTC TTACTATCTC TGGAAATAAA AGTCTGACCT |
| 851 | TCGCCGAGAA CTCTTCAGTA ACTCAAGGCG GAGCAATCTG TGCCCATGGT |
| 901 | CTAGATCTTT CCGCTGCTGG CCCTACCCTA TTTTCAAATA ATAGATGCGG |
| 951 | GAACACAGCT GCAGGCAAGG GCGGCGCTAT TGCAATTGCC GACTCTGGAT |
| 1001 | CTTTAAGTCT CTCTGCAAAT CAAGGAGACA TCACGTTCCT TGGCAACACT |
| 1051 | CTAACCTCAA CCTCCGCGCC AACATCGACA CGGAATGCTA TCTACCTGGG |
| 1101 | ATCGTCAGCA AAAATTACGA ACTTAAGGGC AGCCCAAGGC CAATCTATCT |
| 1151 | ATTTCTATGA TCCGATTGCA TCTAACACCA CAGGAGCTTC AGACGTTCTG |
| 1201 | ACCATCAACC AACCGGATAG CAACTCGCCT TTAGATTATT CAGGAACGAT |
| 1251 | TGTATTTTCT GGGGAAAAGC TCTCTGCAGA TGAAGCGAAA GCTGCTGATA |
| 1301 | ACTTCACATC TATATTAAAG CAACCATTGG CTCTAGCCTC TGGAACCTTA |
| 1351 | GCACTCAAAG GAAATGTCGA GTTAGATGTC AATGGTTTCA CACAGACTGA |
| 1401 | AGGCTCTACA CTCCTCATGC AACCAGGAAC AAAGCTCAAA GCAGATACTG |
| 1451 | AAGCTATCAG TCTTACCAAA CTTGTCGTTG ATCTTTCTGC CTTAGAGGGA |
| 1501 | AATAAGAGTG TGTCCATTGA AACAGCAGGA GCCAACAAAA CTATAACTCT |
| 1551 | AACCTCTCCT CTTGTTTTCC AAGATAGTAG CGGCAATTTT TATGAAAGCC |
| 1601 | ATACGATAAA CCAAGCCTTC ACGCAGCCTT TGGTGGTATT CACTGCTGCT |
| 1651 | ACTGCTGCTA GCGATATTTA TATCGATGCG CTTCTCACTT CTCCAGTACA |
| 1701 | AACTCCAGAA CCTCATTACG GGTATCAGGG ACATTGGGAA GCCACTTGGG |
| 1751 | CAGACACATC AACTGCAAAA TCAGGAACTA TGACTTGGGT AACTACGGGC |
| 1801 | TACAACCCTA ATCCTGAGCG TAGAGCTTCC GTAGTTCCCG ATTCATTATG |
| 1851 | GGCATCCTTT ACTGACATTC GCACTCTACA GCAGATCATG ACATCTCAAG |
| 1901 | CGAATAGTAT CTATCAGCAA CGAGGACTCT GGGCATCAGG AACTGCGAAT |
| 1951 | TTCTTCCATA AGGATAAATC AGGAACTAAC CAAGCATTCC GACATAAAAG |
| 2001 | CTACGGCTAT ATTGTTGGAG GAAGTGCTGA AGATTTTTCT GAAAATATCT |
| 2051 | TCAGTGTAGC TTTCTGCCAG CTCTTCGGTA AAGATAAAGA CCTGTTTATA |
| 2101 | GTTGAAAATA CCTCTCATAA CTATTTAGCG TCGCTATACC TGCAACATCG |
| 2151 | AGCATTCCTA GGAGGACTTC CCATGCCCTC ATTTGGAAGT ATCACCGACA |
| 2201 | TGCTGAAAGA TATTCCTCTC ATTTTGAATG CCCAGCTAAG CTACAGCTAC |
| 2251 | ACTAAAAATG ATATGGATAC TCGCTATACT TCCTATCCTG AAGCTCAAGG |
| 2301 | CTCTTGGACC AATAACTCTG GGGCTCTAGA GCTCGGAGGA TCTCTGGCTC |
| 2351 | TATATCTCCC TAAAGAAGCA CCGTTCTTCC AGGGATATTT CCCCTTCTTA |
| 2401 | AAGTTCCAGG CAGTCTACAG CCGCCAACAA AACTTTAAAG AGAGTGGCGC |
| 2451 | TGAAGCCCGT GCTTTTGATG ATGGAGACCT AGTGAACTGC TCTATCCCTG |
| 2501 | TCGGCATTCG GTTAGAAAAA ATCTCCGAAG ATGAAAAAAA TAATTTCGAG |
| 2551 | ATTTCTCTAG CCTACATTGG TGATGTGTAT CGTAAAAATC CCCGTTCGCG |
| 2601 | TACTTCTCTA ATGGTCAGTG GAGCCTCTTG GACTTCGCTA TGTAAAAACC |
| 2651 | TCGCACGACA AGCCTTCTTA GCAAGTGCTG GAAGCCATCT GACTCTCTCC |
| 2701 | CCTCATGTAG AACTCTCTGG GGAAGCTGCT TATGAGCTTC GTGGCTCAGC |
| 2751 | ACACATCTAC AATGTAGATT GTGGGCTAAG ATACTCATTC TAG |

The PSORT algorithm predicts outer membrane (0.927).

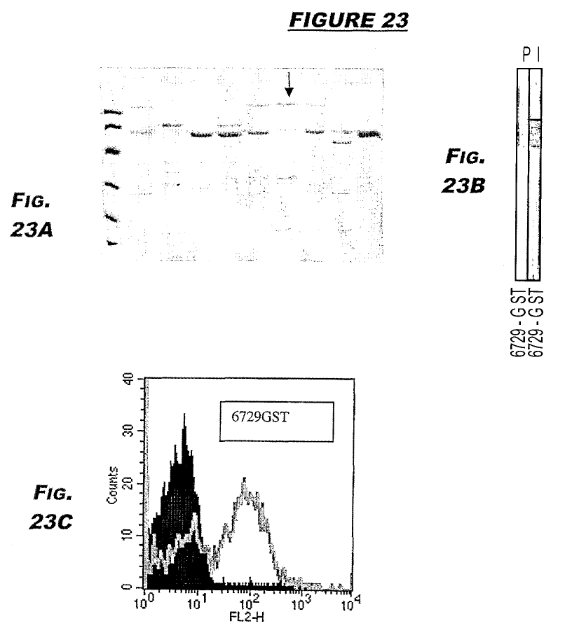

The protein was expressed in E. coli and purified as a GST-fusion product, as shown in FIG. 23A. The recombinant protein was used to immunize mice, whose sera were used in a Western blot (FIG. 23B) and for FACS analysis (FIG. 23C). A his-tag protein was also expressed.

The cp6729 protein was also identified in the 2D-PAGE experiment (Cpn0446) and showed good cross-reactivity with human sera, including sera from patients with pneumonitis.

These experiments show that cp6729 is a surface-exposed and immunoaccessible protein, and that it is a useful immunogen. These properties are not evident from the sequence alone.

### Example 24

The following C. pneumoniae protein (PID 4376849) was expressed <SEQ ID 47; cp6849>:

| | |
|---|---|
| 1 | MSKLIRRVVT VLALTSMASC FASGGIEAAV AESLITKIVA SAETKPAPVP |
| 51 | MTAKKVRLVR RNKQPVEQKS RGAFCDKEFY PCEEGRCQPV EAQQESCYGR |
| 101 | LYSVKVNDDC NVEICQSVPE YATVGSPYPI EILAIGKKDC VDVVITQQLP |
| 151 | CEAEFVSSDP ETTPTSDGKL VWKIDRLGAG DKCKITVWVK PLKEGCCFTA |
| 201 | ATVCACPELR SYTKCGQPAI CIKQEGPDCA CLRCPVCYKI EVVNTGSAIA |
| 251 | RNVTVDNPVP DGYSHASGQR VLSFNLGDMR PGDKKVFTVE FCPQRRGQIT |
| 301 | NVATVTYCGG HKCSANVTTV VNEPCVQVNI SGADWSYVCK PVEYSISVSN |
| 351 | PGDLVLHDVV IQDTLPSGVT VLEAPGGEIC CNKVVWRIKE MCPGETLQFK |
| 401 | LVVKAQVPGR FTNQVAVTSE SNCGTCTSCA ETTTHWKGLA ATHMCVLDTN |
| 451 | DPICVGENTV YRICVTNRGS AEDTNVSLIL KFSKELQPIA SSGPTKGTIS |
| 501 | GNTVVFDALP KLGSKESVEF SVTLKGIAPG DARGEAILSS DTLTSPVSDT |
| 551 | ENTHVY* |

A predicted signal peptide is highlighted.

The cp6849 nucleotide sequence <SEQ ID 48> is:

| | |
|---|---|
| 1 | ATGTCCAAAC TCATCAGACG AGTAGTTACG GTCCTTGCGC TAACGAGTAT |
| 51 | GGCGAGTTGC TTTGCCAGCG GGGGTATAGA GGCCGCTGTA GCAGAGTCTC |
| 101 | TGATTACTAA GATCGTCGCT AGTGCGGAAA CAAAGCCAGC ACCTGTTCCT |
| 151 | ATGACAGCGA AGAAGGTTAG ACTTGTCCGT AGAAATAAAC AACCAGTTGA |
| 201 | ACAAAAAAGC CGTGGTGCTT TTTGTGATAA AGAATTTTAT CCCTGTGAAG |
| 251 | AGGGACGATG TCAACCTGTA GAGGCTCAGC AAGAGTCTTG CTACGGAAGA |
| 301 | TTGTATTCTG TAAAAGTAAA CGATGATTGC AACGTAGAAA TTTGCCAGTC |
| 351 | CGTTCCAGAA TACGCTACTG TAGGATCTCC TTACCCTATT GAAATCCTTG |
| 401 | CTATAGGCAA AAAAGATTGT GTTGATGTTG TGATTACACA ACAGCTACCT |
| 451 | TGCGAAGCTG AATTCGTAAG CAGTGATCCA GAAACAACTC CTACAAGTGA |
| 501 | TGGGAAATTA GTCTGGAAAA TCGATCGCCT GGGTGCAGGA GATAAATGCA |
| 551 | AAATTACTGT ATGGGTAAAA CCTCTTAAAG AAGGTTGCTG CTTCACAGCT |
| 601 | GCTACTGTAT GTGCTTGCCC AGAGCTCCGT TCTTATACTA AATGCGGTCA |
| 651 | ACCAGCCATT TGTATTAAGC AAGAAGGACC TGACTGTGCT TGCCTAAGAT |
| 701 | GCCCTGTATG CTACAAAATC GAAGTAGTGA ACACAGGATC TGCTATTGCC |
| 751 | CGTAACGTAA CTGTAGATAA TCCTGTTCCC GATGGCTATT CTCATGCATC |
| 801 | TGGTCAAAGA GTTCTCTCTT TTAACTTAGG AGACATGAGA CCTGGCGATA |
| 851 | AAAAGGTATT TACAGTTGAG TTCTGCCCTC AAAGAAGAGG TCAAATCACT |
| 901 | AACGTTGCTA CTGTAACTTA CTGCGGTGGA CACAAATGTT CTGCAAATGT |
| 951 | AACTACAGTT GTTAATGAGC CTTGTGTACA AGTAAATATC TCTGGTGCTG |
| 1001 | ATTGGTCTTA CGTATGTAAA CCTGTGGAGT ACTCTATCTC AGTATCGAAT |
| 1051 | CCTGGAGACT TGGTTCTTCA TGATGTCGTG ATCCAAGATA CACTCCCTTC |
| 1101 | TGGTGTTACA GTACTCGAAG CTCCTGGTGG AGAGATCTGC TGTAATAAAG |
| 1151 | TTGTTTGGCG TATTAAAGAA ATGTGCCCAG GAGAAACCCT CCAGTTTAAA |
| 1201 | CTTGTAGTGA AAGCTCAAGT TCCTGGAAGA TTCACAAATC AAGTTGCAGT |
| 1251 | AACTAGTGAG TCTAACTGCG GAACATGTAC ATCTTGCGCA GAAACAACAA |
| 1301 | CACATTGGAA AGGTCTTGCA GCTACCCATA TGTGCGTATT AGACACAAAT |
| 1351 | GATCCTATCT GTGTAGGAGA AAATACTGTC TATCGTATCT GTGTAACTAA |
| 1401 | CCGTGGTTCT GCTGAAGATA CTAACGTATC TTTAATCTTG AAGTTCTCAA |
| 1451 | AAGAACTTCA GCCAATAGCT TCTTCAGGTC CAACTAAAGG AACGATTTCA |
| 1501 | GGTAATACCG TTGTTTTCGA CGCTTTACCT AAACTCGGTT CTAAGGAATC |
| 1551 | TGTAGAGTTT TCTGTTACCT TGAAAGGTAT TGCTCCCGGA GATGCTCGCG |
| 1601 | GCGAAGCTAT TCTTTCTTCT GATACACTGA CTTCACCAGT ATCAGACACA |
| 1651 | GAAAATACCC ACGTGTATTA A |

The PSORT algorithm predicts periplasmic space (0.93).

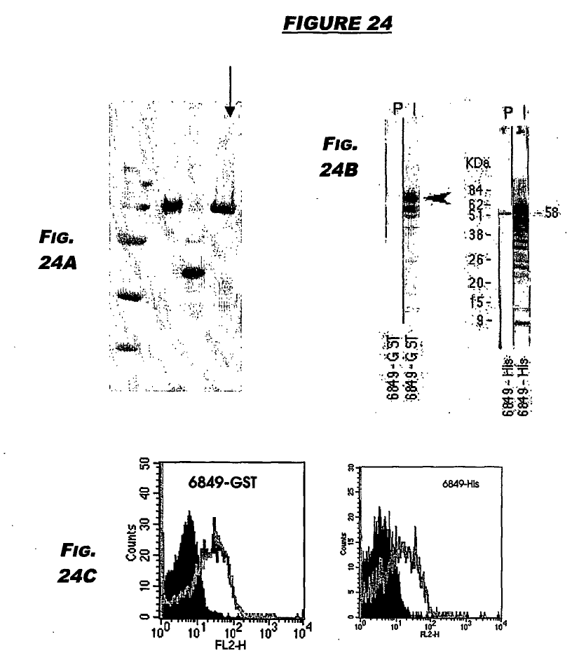

The protein was expressed in E. coli and purified as a GST-fusion product, as shown in FIG. 24A, and also as a his-tag protein. The recombinant proteins were used to immunize mice, whose sera were used in a Western blot (FIG. 24B) and for FACS analysis (FIG. 24C).

The cp6849 protein was also identified in the 2D-PAGE experiment (Cpn0557).

These experiments show that cp6849 is a surface-exposed and immunoaccessible protein, and that it is a useful immunogen. These properties are not evident from the sequence alone.

### Example 25

The following C. pneumoniae protein (PID 4376273) was expressed <SEQ ID 49; cp6273>:

| | |
|---|---|
| 1 | MGLFHLTLFG LLLCSLPISL VAKFPESVGH KILYISTQST QQALATYLEA |
| 51 | LDAYGDHDFF VLRKIGEDYL KQSIHSSDPQ TRKSTIIGAG LAGSSEALDV |
| 101 | LSQAMETADP LQQLLVLSAV SGHLGKTSDD LLFKALASPY PVIRLEAAYR |
| 151 | LANLKNTKVI DHLHSFIHKL PEEIQCLSAA IFLRLETEES DAYIRDLLAA |
| 201 | KKSAIRSATA LQIGEYQQKR FLPTLRNLLT SASPQDQEAI LYALGKLKDG |
| 251 | QSYYNIKKQL QKPDVDVTLA AAQALIALGK EEDALPVIKK QALEERPRAL |
| 301 | YALRHLPSEI GIPIALPIFL KTKNSEAKLN VALALLELGC DTPKLLEYIT |
| 351 | ERLVQPHYNE TLALSFSKGR TLQNWKRVNI IVPQDPQERE RLLSTTRGLE |
| 401 | EQILTFLFRL PKEAYLPCIY KLLASQKTQL ATTAISFLSH TSHQEALDLL |
| 451 | FQAAKLPGEP IIRAYADLAI YNLTKDPEKK RSLHDYAKKL IQETLLFVDT |
| 501 | ENQRPHPSMP YLRYQVTPES RTKLMLDILE TLATSKSSED IRLLIQLMTE |
| 551 | GDAKNFPVLA GLLIKIVE* |

A predicted signal peptide is highlighted.

The cp6273 nucleotide sequence <SEQ ID 50> is:

| | |
|---|---|
| 1 | ATGGGACTAT TCCATCTAAC TCTCTTTGGA CTTTTATTGT GTAGTCTTCC |
| 51 | CATTTCTCTT GTTGCTAAAT TCCCTGAGTC TGTAGGTCAT AAGATCCTTT |
| 101 | ATATAAGTAC GCAATCTACA CAGCAGGCCT TAGCAACATA TCTGGAAGCT |
| 151 | CTAGATGCCT ACGGTGATCA TGACTTCTTC GTTTTAAGAA AAATCGGAGA |
| 201 | AGACTATCTC AAGCAAAGCA TCCACTCCTC AGATCCGCAA ACTAGAAAAA |
| 251 | GCACCATCAT TGGAGCAGGC CTGGCGGGAT CTTCAGAAGC CTTGGACGTG |
| 301 | CTCTCCCAAG CTATGGAAAC TGCAGACCCC CTGCAGCAGC TACTGGTTTT |
| 351 | ATCGGCAGTC TCAGGACATC TTGGGAAAAC TTCTGACGAC TTACTGTTTA |
| 401 | AAGCTTTAGC ATCTCCCTAT CCTGTCATCC GCTTAGAAGC CGCCTATAGA |
| 451 | CTTGCTAATT TGAAGAACAC TAAAGTCATT GATCATCTAC ATTCTTTCAT |
| 501 | TCATAAGCTT CCCGAAGAAA TCCAATGCCT ATCTGCGGCA ATATTCCTAC |
| 551 | GCTTGGAGAC TGAAGAATCT GATGCTTATA TTCGGGATCT CTTAGCTGCC |
| 601 | AAGAAAAGCG CGATTCGGAG TGCCACAGCT TTGCAGATCG GAGAATACCA |
| 651 | ACAAAAACGC TTTCTTCCGA CACTTAGGAA TTTGCTAACG AGTGCGTCTC |
| 701 | CTCAAGATCA AGAAGCTATT CTTTATGCTT TAGGGAAGCT TAAGGATGGT |
| 751 | CAGAGCTACT ACAATATAAA AAAGCAATTG CAGAAGCCTG ATGTGGATGT |
| 801 | CACTTTAGCA GCAGCTCAAG CTTTAATTGC TTTGGGGAAA GAAGAGGACG |
| 851 | CTCTTCCCGT GATAAAAAAG CAAGCACTTG AGGAGCGGCC TCGAGCCCTG |
| 901 | TATGCCTTAC GGCATCTACC CTCTGAGATA GGGATTCCGA TTGCCCTGCC |
| 951 | GATATTCCTA AAAACTAAGA ACAGCGAAGC CAAGTTGAAT GTAGCTTTAG |
| 1001 | CTCTCTTAGA GTTAGGGTGT GACACCCCTA AACTACTGGA ATACATTACC |
| 1051 | GAAAGGCTTG TCCAACCACA TTATAATGAG ACTCTAGCCT TGAGTTTCTC |
| 1101 | TAAGGGGCGT ACTTTACAAA ATTGGAAGCG GGTGAACATC ATAGTCCCTC |
| 1151 | AAGATCCCCA GGAGAGGGAA AGGTTGCTCT CCACAACCCG AGGTCTTGAA |
| 1201 | GAGCAGATCC TTACGTTTCT CTTCCGCCTA CCTAAAGAAG CTTACCTCCC |
| 1251 | CTGTATTTAT AAGCTTTTGG CGAGTCAGAA AACTCAGCTT GCCACTACTG |
| 1301 | CGATTTCTTT TTTAAGTCAC ACCTCACATC AGGAAGCCTT AGATCTACTT |
| 1351 | TTCCAAGCTG CGAAGCTTCC TGGAGAACCT ATCATCCGCG CCTATGCAGA |
| 1401 | TCTTGCTATT TATAATCTCA CCAAAGATCC TGAAAAAAAA CGTTCTCTCC |
| 1451 | ATGATTATGC AAAAAAGCTA ATTCAGGAAA CCTTGTTATT TGTGGACACG |
| 1501 | GAAAACCAAA GACCCCATCC CAGCATGCCC TATCTACGTT ATCAGGTCAC |
| 1551 | CCCAGAAAGC CGTACGAAGC TCATGTTGGA TATTCTAGAG ACACTAGCCA |
| 1601 | CCTCGAAGTC TTCCGAAGAT ATCCGTTTAT TGATACAACT GATGACGGAA |
| 1651 | GGAGATGCAA AAAATTTCCC AGTCCTTGCA GGCTTACTCA TAAAAATTGT |
| 1701 | GGAGTAA |

The PSORT algorithm predicts a periplasmic location (0.922).

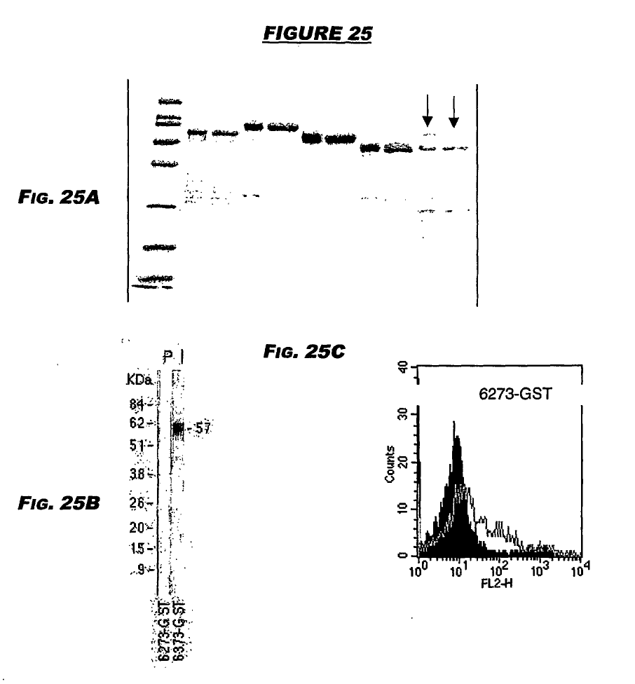

The protein was expressed in E. coli and purified as a his-tag product and as a GST-fusion product, as shown in FIG. 25A. The recombinant GST-fusion was used to immunize mice, whose sera were used in a Western blot (FIG. 25B) and for FACS analysis (FIG. 25C).

This protein also showed good cross-reactivity with human sera, including sera from patients with pneumonitis.

These experiments show that cp6273 is a surface-exposed and immunoaccessible protein, and that it is a useful immunogen. These properties are not evident from the sequence alone.

### Example 26

The following C. pneumoniae protein (PID 4376735) was expressed <SEQ ID 51; cp6735>:

| | |
|---|---|
| 1 | MTILRNFLTC SALFLALPAA AQVVYLHESD GYNGAINNKS LEPKITCYPE |
| 51 | GTSYIFLDDV RISNVKHDQE DAGVFINRSG NLFFMGNRCN FTFHNLMTEG |
| 101 | FGAAISNRVG DTTLTLSNFS YLAFTSAPLL PQGQGAIYSL GSVMIENSEE |
| 151 | VTFCGNYSSW SGAAIYTPYL LGSKASRPSV NLSGNRYLVF RDNVSQGYGG |
| 201 | AISTHNLTLT TRGPSCFENN HAYHDVNSNG GAIAIAPGGS ISISVKSGDL |
| 251 | IFKGNTASQD GNTIHNSIHL QSGAQFKNLR AVSESGVYFY DPISHSESHK |
| 301 | ITDLVINAPE GKETYEGTIS FSGLCLDDHE VCAENLTSTI LQDVTLAGGT |
| 351 | LSLSDGVTLQ LHSFKQEASS TLTMSPGTTL LCSGDARVQN LHILIEDTDN |
| 401 | FVPVRIRAED KDALVSLEKL KVAFEAYWSV YDFPQFKEAF TIPLLELLGP |
| 451 | SFDSLLLGET TLERTQVTTE NDAVRGFWSL SWEEYPPSLD KDRRITPTKK |
| 501 | TVFLTWNPEI TSTP* |

A predicted signal peptide is highlighted.

The cp6735 nucleotide sequence <SEQ ID 52> is:

| | |
|---|---|
| 1 | ATGACCATAC TTCGAAATTT TCTTACCTGC TCGGCTTTAT TCCTCGCTCT |
| 51 | CCCTGCAGCA GCACAAGTTG TATATCTTCA TGAAAGTGAT GGTTATAACG |
| 101 | GTGCTATCAA TAATAAAAGC TTAGAACCTA AAATTACCTG TTATCCAGAA |
| 151 | GGAACTTCTT ACATCTTTCT AGATGACGTG AGGATTTCCA ACGTTAAGCA |
| 201 | TGATCAAGAA GATGCTGGGG TTTTTATAAA TCGATCTGGG AATCTTTTTT |
| 251 | TCATGGGCAA CCGTTGCAAC TTCACTTTTC ACAACCTTAT GACCGAGGGT |
| 301 | TTTGGCGCTG CCATTTCGAA CCGCGTTGGA GACACCACTC TCACTCTCTC |
| 351 | TAATTTTTCT TACTTAGCGT TCACCTCAGC ACCTCTACTA CCTCAAGGAC |
| 401 | AAGGAGCGAT TTATAGTCTT GGTTCCGTGA TGATCGAAAA TAGTGAGGAA |
| 451 | GTGACTTTCT GTGGGAACTA CTCTTCGTGG AGTGGAGCTG CGATTTATAC |
| 501 | TCCCTACCTT TTAGGTTCTA AGGCGAGTCG TCCTTCAGTA AATCTCAGCG |
| 551 | GGAACCGCTA CCTGGTGTTT AGAGACAATG TGAGCCAAGG TTATGGCGGC |
| 601 | GCCATATCTA CCCACAATCT CACACTCACG ACTCGAGGAC CTTCGTGTTT |
| 651 | TGAAAATAAT CATGCTTATC ATGACGTGAA TAGTAATGGA GGAGCCATTG |
| 701 | CCATTGCTCC TGGAGGATCG ATCTCTATAT CCGTGAAAAG CGGAGATCTC |
| 751 | ATCTTCAAAG GAAATACAGC ATCACAAGAC GGAAATACAA TACACAACTC |
| 801 | CATCCATCTG CAATCTGGAG CACAGTTTAA GAACCTACGT GCTGTTTCAG |
| 851 | AATCCGGAGT TTATTTCTAT GATCCTATAA GCCATAGCGA GTCGCATAAA |
| 901 | ATTACAGATC TTGTAATCAA TGCTCCTGAA GGAAAGGAAA CTTATGAAGG |
| 951 | AACAATTAGC TTCTCAGGAC TATGCCTGGA TGATCATGAA GTTTGTGCGG |
| 1001 | AAAATCTTAC TTCCACAATC CTACAAGATG TCACATTAGC AGGAGGAACT |
| 1051 | CTCTCTCTAT CGGATGGGGT TACCTTGCAA CTGCATTCTT TTAAGCAGGA |
| 1101 | AGCAAGCTCT ACGCTTACTA TGTCTCCAGG AACCACTCTG CTCTGCTCAG |
| 1151 | GAGATGCTCG GGTTCAGAAT CTGCACATCC TGATTGAAGA TACCGACAAC |
| 1201 | TTTGTTCCTG TAAGGATTCG CGCCGAGGAC AAGGATGCTC TTGTCTCATT |
| 1251 | AGAAAAACTT AAAGTTGCCT TTGAGGCTTA TTGGTCCGTC TATGACTTTC |
| 1301 | CTCAATTTAA GGAAGCCTTT ACGATTCCTC TTCTTGAACT TCTAGGGCCT |
| 1351 | TCTTTTGACA GTCTTCTCCT AGGGGAGACC ACTTTGGAGA GAACCCAAGT |
| 1401 | CACAACAGAG AATGACGCCG TTCGAGGTTT CTGGTCCCTA AGCTGGGAAG |
| 1451 | AGTACCCCCC TTCTCTGGAT AAAGACAGAA GGATCACACC AACTAAGAAA |
| 1501 | ACTGTTTTCC TCACTTGGAA TCCTGAGATC ACTTCTACGC CATAA |

The PSORT algorithm predicts an outer membrane location (0.922).

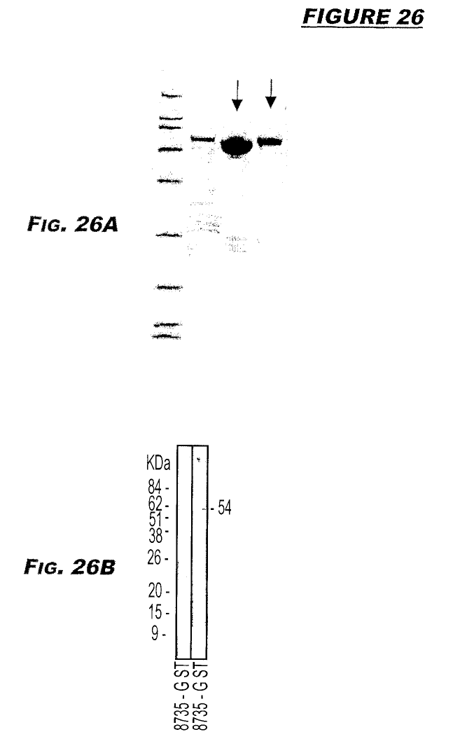

The protein was expressed in E. coli and purified as a as a his-tag product and as a GST-fusion product, as shown in FIG. 26A. The recombinant GST-fusion protein was used to immunize mice, whose sera were used in a Western blot (FIG. 26B).

These experiments show that cp6735 is a surface-exposed and immunoaccessible protein, and that it is a useful immunogen. These properties are not evident from the sequence alone.

### Example 27

The following C. pneumoniae protein (PID 4376784) was expressed <SEQ ID 53; cp6784>:

| | |
|---|---|
| 1 | MNRRKARWVV ALFAMTALIS VGCCPWSQAK SRCSIDKYIP VVNRLLEVCG |
| 51 | LPEAENVEDL IESSSAWVLT PEERFSGELV SICQVKDEHA FYNDLSLLHM |
| 101 | TQAVPSYSAT YDCAVVFGGP LPALRQRLDF LVREWQRGVR FKKIVFLCGE |
| 151 | RGRYQSIEEQ EHFFDSRYNP FPTEENWESG NRVTPSSEEE IAKFVWMQML |
| 201 | LPRAWRDSTS GVRVTFLLAK PEENRVVANR KDTLLLFRSY QEAFPGRVLF |
| 251 | VSSQPFIGLD ACRVGQFFKG ESYDLAGPGF AQGVLKYHWA PRICLHTLAE |
| 301 | WLKETNGCLN ISEGCFG* |

A predicted signal peptide is highlighted.

The cp6784 nucleotide sequence <SEQ ID 54> is:

| | |
|---|---|
| 1 | ATGAATAGAA GAAAAGCAAG ATGGGTAGTG GCATTGTTCG CAATGACGGC |
| 51 | GCTCATTTCT GTTGGGTGTT GTCCTTGGTC ACAAGCGAAA TCAAGATGTT |
| 101 | CTATTGATAA GTATATTCCT GTAGTCAATC GTTTACTAGA AGTTTGTGGA |
| 151 | CTTCCTGAAG CTGAGAATGT TGAGGATTTA ATCGAGTCCT CGTCTGCTTG |
| 201 | GGTACTGACT CCTGAAGAAC GTTTTTCTGG AGAGTTAGTC TCTATCTGTC |
| 251 | AGGTTAAAGA TGAGCATGCT TTCTATAACG ATTTGTCTTT ATTACATATG |
| 301 | ACTCAGGCTG TGCCTTCGTA TTCTGCAACG TATGATTGTG CTGTAGTTTT |
| 351 | TGGCGGGCCT TTGCCAGCGC TACGTCAGCG CTTAGATTTT TTGGTGCGAG |
| 401 | AGTGGCAGCG TGGCGTGCGC TTTAAGAAAA TCGTTTTTCT ATGTGGAGAG |
| 451 | CGAGGGCGCT ATCAGTCTAT TGAAGAACAA GAGCATTTCT TTGATTCTCG |
| 501 | GTACAATCCT TTCCCTACTG AAGAGAACTG GGAATCTGGT AACCGAGTTA |
| 551 | CTCCCTCTTC TGAAGAAGAG ATTGCCAAAT TTGTTTGGAT GCAAATGCTT |
| 601 | TTACCTAGAG CATGGCGAGA TAGTACTTCA GGAGTCAGAG TGACATTTCT |
| 651 | TCTAGCAAAG CCAGAGGAAA ATCGTGTGGT TGCGAATCGT AAGGACACCT |
| 701 | TACTTTTATT CCGTTCTTAT CAAGAAGCGT TTCCGGGACG CGTGTTATTT |
| 751 | GTAAGTAGTC AACCCTTTAT CGGTTTAGAT GCTTGCAGGG TCGGGCAGTT |
| 801 | TTTCAAAGGG GAAAGCTATG ATCTTGCTGG ACCTGGATTT GCTCAAGGAG |
| 851 | TCTTGAAGTA TCATTGGGCT CCAAGGATTT GTCTACATAC TTTAGCGGAA |
| 901 | TGGTTAAAGG AAACGAACGG CTGCTTAAAT ATTTCAGAGG GTTGTTTTGG |
| 951 | ATGA |

The PSORT algorithm predicts a periplasmic location (0.894).

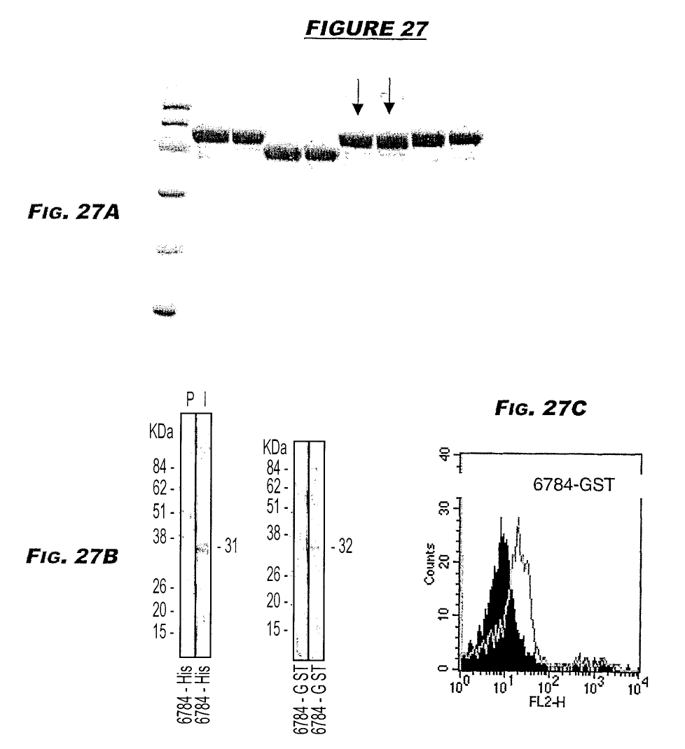

The protein was expressed in E. coli and purified as a his-tag product and as a GST-fusion product, as shown in FIG. 27A. The recombinant proteins were used to immunize mice, whose sera were used in a Western blot (FIG. 27B). The GST-fusion product was used for FACS analysis (FIG. 27C).

The cp6784 protein was also identified in the 2D-PAGE experiment (Cpn0498).

These experiments show that cp6784 is a surface-exposed and immunoaccessible protein, and that it is a useful immunogen. These properties are not evident from the sequence alone.

### Example 28

The following C. pneumoniae protein (PID 4376960) was expressed <SEQ ID 55; cp6960>:

| | |
|---|---|
| 1 | MNRRWNLVLA TVALALSVAS CDVRSKDKDK DQGSLVEYKD NKDTNDIELS |
| 51 | DNQKLSRTFG HLLARQLRKS EDMFFDIAEV AKGLQAELVC KSAPLTETEY |
| 101 | EEKMAEVQKL VFEKKSKENL SLAEKFLKEN SKNAGVVEVQ PSKLQYKIIK |
| 151 | EGAGKAISGK PSALLHYKGS FINGQVFSSS EGNNEPILLP LGQTIPGFAL |
| 201 | GMQFMKEGET FVLYIHPDLA YGTAGQLPPN SLLIFEINLI QASADEVAAV |
| 251 | PQEGNQGE* |

A predicted signal peptide is highlighted.

The cp6960 nucleotide sequence <SEQ ID 56> is:

| | |
|---|---|
| 1 | ATGAACAGAC GGTGGAATTT AGTTTTAGCA ACAGTAGCTC TGGCACTCTC |
| 51 | CGTCGCTTCT TGTGACGTAC GGTCTAAGGA TAAAGACAAG GATCAGGGGT |
| 101 | CGTTAGTGGA ATATAAAGAT AACAAAGATA CCAATGACAT AGAATTATCC |
| 151 | GATAATCAAA AGTTATCCAG AACATTTGGT CATTTATTAG CACGCCAATT |
| 201 | ACGCAAGTCA GAAGATATGT TTTTTGATAT TGCAGAAGTG GCTAAGGGGT |
| 251 | TGCAGGCGGA ATTGGTTTGT AAAAGTGCTC CTTTAACAGA AACAGAGTAT |
| 301 | GAAGAAAAAA TGGCTGAAGT ACAGAAGTTG GTTTTTGAAA AAAAATCAAA |
| 351 | AGAAAATCTT TCATTGGCAG AAAAATTCTT AAAAGAAAAT AGCAAGAACG |
| 401 | CTGGTGTTGT TGAAGTGCAA CCAAGTAAAT TGCAATACAA AATTATTAAA |
| 451 | GAAGGTGCAG GGAAAGCAAT TTCAGGTAAA CCTTCAGCTC TATTGCACTA |
| 501 | CAAGGGTTCC TTCATCAATG GCCAAGTATT TAGCAGTTCA GAAGGCAACA |
| 551 | ATGAGCCTAT CTTGCTTCCT CTAGGCCAAA CAATTCCTGG TTTTGCTTTA |
| 601 | GGTATGCAGG GCATGAAAGA AGGAGAAACT CGAGTTCTCT ACATCCATCC |
| 651 | TGATCTTGCT TACGGAACCG CAGGACAACT TCCTCCAAAC TCTTTATTAA |
| 701 | TTTTTGAAAT TAACTTGATT CAGGCTTCAG CAGATGAAGT TGCTGCTGTA |
| 751 | CCCCAAGAAG GAAATCAAGG TGAATGA |

The PSORT algorithm predicts periplasmic space location (0.930).

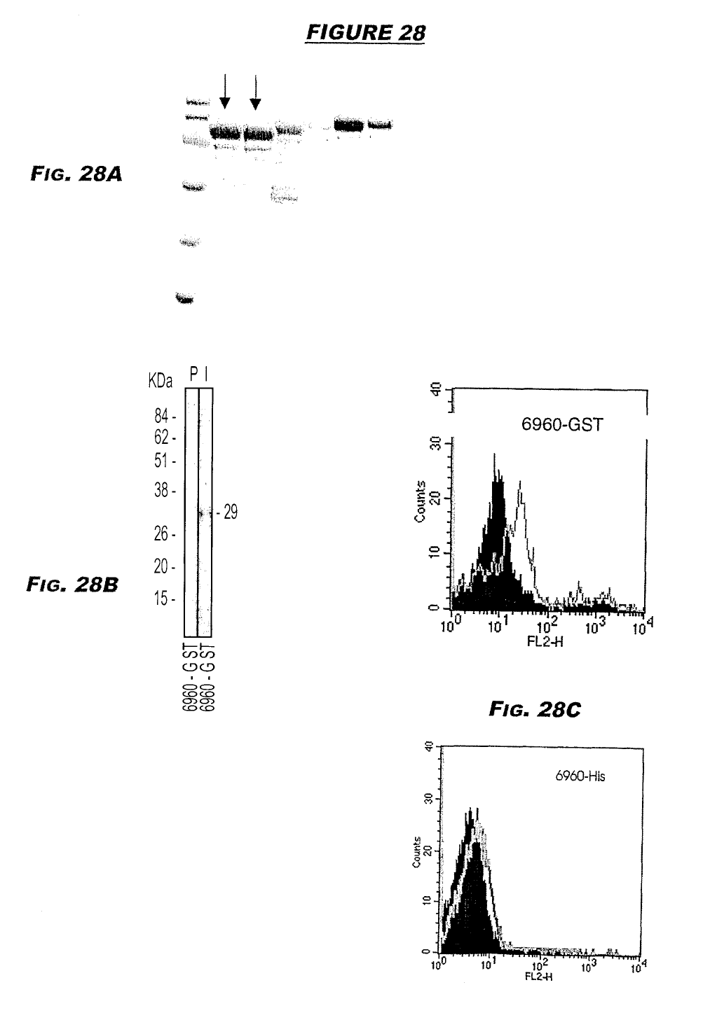

The protein was expressed in E. coli and purified as a his-tag product and as a GST-fusion product, as shown in FIG. 28A. The recombinant proteins were used to immunize mice, whose sera were used in a Western blot (FIG. 28B) and for FACS analysis (FIG. 28C).

The cp6960 protein was also identified in the 2D-PAGE experiment.

These experiments show that cp6960 is a surface-exposed and immunoaccessible protein, and that it is a useful immunogen. These properties are not evident from the sequence alone.

### Example 29

The following C. pneumoniae protein (PID 4376968) was expressed <SEQ ID 57; cp6968>:

| | |
|---|---|
| 1 | MKFLLYVPLL LVLVSTGCDA KPVSFEPFSG KLSTQRFEPQ HSAEEYFSQG |
| 51 | QEFLKKGNFR KALLCFGIIT HHFPRDILRN QAQYLIGVCY FTQDHPDLAD |
| 101 | KAFASYLQLP DAEYSEELFQ MKYAIAQRFA QGKRKRICRL EGFPKLMNAD |
| 151 | EDALRIYDEI LTAFPSKDLG AQALYSKAAL LIVKNDLTEA TKTLKKLTLQ |
| 201 | FPLHILSSEA FVRLSEIYLQ QAKKEPHNLQ YLHFAKLNEE AMKKQHPNHP |
| 251 | LNEVVSANVG AMREHYARGL YATGRFYEKK KKAEAANIYY RTAITNYPDT |
| 301 | LLVAKCQKRL DRISKHTS* |

A predicted signal peptide is highlighted.

The cp6968 nucleotide sequence <SEQ ID 58> is:

| | |
|---|---|
| 1 | ATGAAATTTC TATTATACGT TCCACTTCTT CTTGTTCTCG TATCTACGGG |
| 51 | GTGCGATGCA AAACCTGTTT CTTTTGAGCC CTTTTCAGGA AAGCTTTCCA |
| 101 | CCCAGCGTTT TGAGCCTCAG CACTCTGCTG AAGAATATTT TTCTCAGGGA |
| 151 | CAGGAATTCT TAAAAAAAGG AAATTTCAGA AAAGCTTTAC TATGCTTTGG |
| 201 | AATCATTACG CATCACTTCC CTAGGGACAT CTTGCGTAAT CAAGCACAGT |
| 251 | ATCTTATAGG AGTCTGTTAC TTCACGCAGG ATCACCCAGA TTTAGCAGAC |
| 301 | AAGGCATTTG CATCTTACTT ACAACTTCCT GATGCGGAGT ACTCTGAAGA |
| 351 | GTTGTTCCAG ATGAAATATG CGATTGCTCA AAGATTTGCT CAAGGGAAGC |
| 401 | GTAAACGGAT TTGTCGATTA GAGGGCTTCC CAAAACTAAT GAATGCTGAT |
| 451 | GAAGATGCGC TACGCATTTA TGACGAGATT CTAACAGCGT TTCCTAGTAA |
| 501 | AGACTTAGGA GCTCAGGCCC TCTATAGTAA AGCTGCGTTA CTTATTGTAA |
| 551 | AAAACGATCT TACAGAAGCC ACCAAAACCT TAAAAAAACT CACGTTACAA |
| 601 | TTTCCTCTAC ATATTTTATC TTCAGAGGCC TTTGTACGTT TATCGGAAAT |
| 651 | CTATTTACAG CAAGCTAAGA AAGAGCCTCA CAATCTTCAA TATCTTCATT |
| 701 | TTGCAAAGCT TAATGAAGAG GCAATGAAAA AGCAGCATCC TAACCATCCT |
| 751 | CTGAATGAGG TTGTTTCTGC TAATGTTGGA GCTATGCGGG AACATTATGC |
| 801 | TCGAGGTTTG TATGCCACAG GTCGTTTCTA TGAGAAGAAG AAAAAAGCCG |
| 851 | AGGCTGCGAA TATCTATTAC CGCACTGCGA TTACAAACTA CCCAGACACT |
| 901 | TTATTAGTGG CTAAATGTCA AAAGCGTCTA GATAGAATAT CTAAGCATAC |
| 951 | TTCCTAA |

The PSORT algorithm predicts an inner membrane location (0.790).

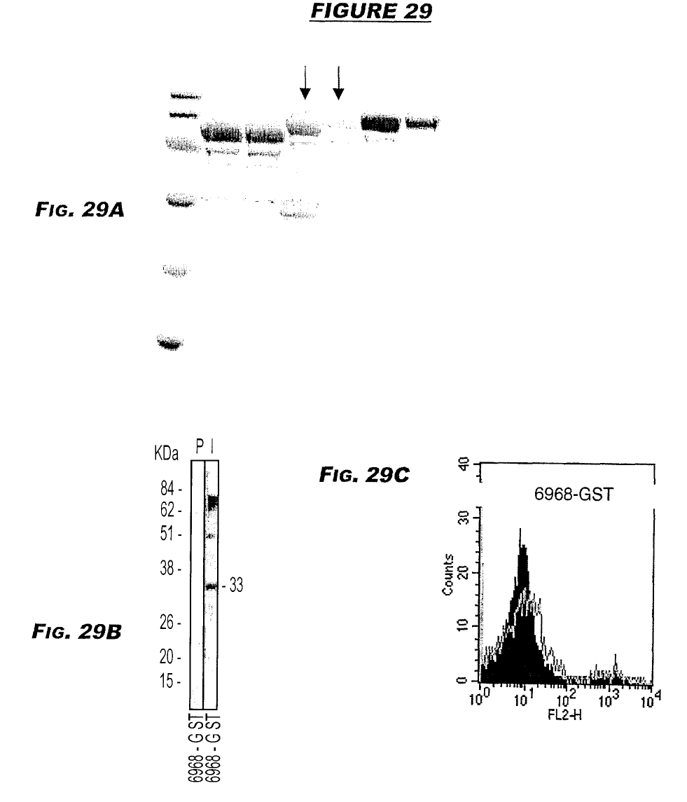

The protein was expressed in E. coli and purified as a his-tag product and as a GST-fusion product, as shown in FIG. 29A. The recombinant GST-fusion was used to immunize mice, whose sera were used in a Western blot (FIG. 29B) and for FACS analysis (FIG. 29C).

This protein also showed good cross-reactivity with human sera, including sera from patients with pneumonitis.

These experiments show that cp6968 is a surface-exposed and immunoaccessible protein, and that it is a useful immunogen. These properties are not evident from the sequence alone.

### Example 30

The following C. pneumoniae protein (PID 4376998) was expressed <SEQ ID 59; cp6998>:

| | |
|---|---|
| 1 | MKKLLKSALL SAAFAGSVGS LQALPVGNPS DPSLLIDGTI WEGAAGDPCD |
| 51 | PCATWCDAIS LRAGFYGDYV FDRILKVDAP KTFSMGAKPT GSAAANYTTA |
| 101 | VDRPNPAYNK HLHDAEWFTN AGFIALNIWD RFDVFCTLGA SNGYIRGNST |
| 151 | AFNLVGLFGV KGTTVNANEL PNVSLSNGVV ELYTDTSFSW SVGARGALWE |
| 201 | CGCATLGAEF QYAQSKPKVE ELNVICNVSQ FSVNKPKGYK GVAFPLPTDA |
| 251 | GVATATGTKS ATINYHEWQV GASLSYRLNS LVPYIGVQWS RATFDADNIR |
| 301 | IAQPKLPTAV LNLTAWNPSL LGNATALSTT DSFSDFMQIV SCQINKFKSR |
| 351 | KACGVTVGAT LVDADKWSLT AEARLINERA AHVSGQFRF* |

A predicted signal peptide is highlighted.

The cp6998 nucleotide sequence <SEQ ID 60> is:

| | |
|---|---|
| 1 | ATGAAAAAAC TCTTAAAGTC GGCGTTATTA TCCGCCGCAT TTGCTGGTTC |
| 51 | TGTTGGCTCC TTACAAGCCT TGCCTGTAGG GAACCCTTCT GATCCAAGCT |
| 101 | TATTAATTGA TGGTACAATA TGGGAAGGTG CTGCAGGAGA TCCTTGCGAT |
| 151 | CCTTGCGCTA CTTGGTGCGA CGCTATTAGC TTACGTGCTG GATTTTACGG |
| 201 | AGACTATGTT TTCGACCGTA TCTTAAAAGT AGATGCACCT AAAACATTTT |
| 251 | CTATGGGAGC CAAGCCTACT GGATCCGCTG CTGCAAACTA TACTACTGCC |
| 301 | GTAGATAGAC CTAACCCGGC CTACAATAAG CATTTACACG ATGCAGAGTG |
| 351 | GTTCACTAAT GCAGGCTTCA TTGCCTTAAA CATTTGGGAT CGCTTTGATG |
| 401 | TTTTCTGTAC TTTAGGAGCT TCTAATGGTT ACATTAGAGG AAACTCTACA |
| 451 | GCGTTCAATC TCGTTGGTTT ATTCGGAGTT AAAGGTACTA CTGTAAATGC |
| 501 | AAATGAACTA CCAAACGTTT CTTTAAGTAA CGGAGTTGTT GAACTTTACA |
| 551 | CAGACACCTC TTTCTCTTGG AGCGTAGGCG CTCGTGGAGC CTTATGGGAA |
| 601 | TGCGGTTGTG CAACTTTGGG AGCTGAATTC CAATATGCAC AGTCCAAACC |
| 651 | TAAAGTTGAA GAACTTAATG TGATCTGTAA CGTATCGCAA TTCTCTGTAA |
| 701 | ACAAACCCAA GGGCTATAAA GGCGTTGCTT TCCCCTTGCC AACAGACGCT |
| 751 | GGCGTAGCAA CAGCTACTGG AACAAAGTCT GCGACCATCA ATTATCATGA |
| 801 | ATGGCAAGTA GGAGCCTCTC TATCTTACAG ACTAAACTCT TTAGTGCCAT |
| 851 | ACATTGGAGT ACAATGGTCT CGAGCAACTT TTGATGCTGA TAACATCCGC |
| 901 | ATTGCTCAGC CAAAACTACC TACAGCTGTT TTAAACTTAA CTGCATGGAA |
| 951 | CCCTTCTTTA CTAGGAAATG CCACAGCATT GTCTACTACT GATTCGTTCT |
| 1001 | CAGACTTCAT GCAAATTGTT TCCTGTCAGA TCAACAAGTT TAAATCTAGA |
| 1051 | AAAGCTTGTG GAGTTACTGT AGGAGCTACT TTAGTTGATG CTGATAAATG |
| 1101 | GTCACTTACT GCAGAAGCTC GTTTAATTAA CGAGAGAGCT GCTCACGTAT |
| 1151 | CTGGTCAGTT CAGATTCTAA |

The PSORT algorithm predicts an outer membrane location (0.707).

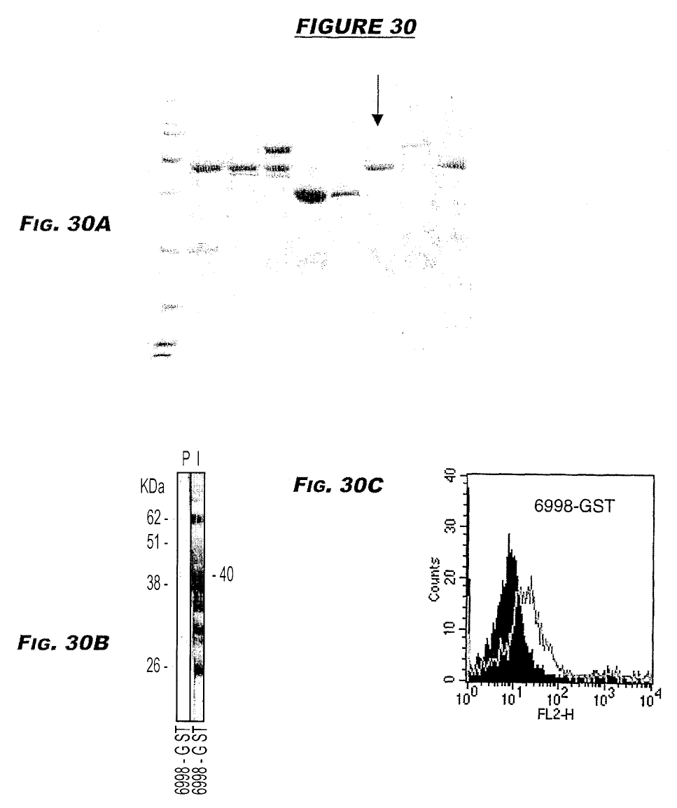

The protein was expressed in E. coli and purified as a GST-fusion (FIG. 30A) and as a his-tag product. The recombinant GST-fusion protein was used to immunize mice, whose sera were used in a Western blot (FIG. 30B) and for FACS analysis (FIG. 30C).

The cp6998 protein was also identified in the 2D-PAGE experiment (Cpn0695) and showed good cross-reactivity with human sera, including sera from patients with pneumonitis.

These experiments show that cp6998 is a surface-exposed and immunoaccessible protein, and that it is a useful immunogen. These properties are not evident from the sequence alone.

### Example 31

The following C. pneumoniae protein (PID 4377102) was expressed <SEQ ID 61; cp7102>:

| | |
|---|---|
| 1 | MKHTFTKRVL FFFFLVIPIP LLLNLMVVGF FSFSAAKANL VQVLHTRATN |
| 51 | LSIEFEKKLT IHKLFLDRLA NTLALKSYAS PSAEPYAQAY NEMMALSNTD |
| 101 | FSLCLIDPFD GSVRTKNPGD PFIRYLKQHP EMKKKLSAAV GKAFLLTIPG |
| 151 | KPLLHYLILV EDVASWDSTT TSGLLVSFYP MSFLQKDLFQ SLHITKGNIC |
| 201 | LVNKYGEVLF CAQDSESSFV FSLDLPNLPQ FQARSPSAIE IEKASGILGG |
| 251 | ENLITVSINK KRYLGLVLNK IPIQGTYTLS LVPVSDLIQS ALKVPLNICF |
| 301 | FYVLAFLLMW WIFSKINTKL NKPLQELTFC MEAAWRGNHN VRFEPQPYGY |
| 351 | EFNELGNIFN CTLLLLLNSI EKADIDYHSG EKLQKELGIL SSLQSALLSP |
| 401 | DFPTFPKVTF SSQHLRRRQL SGHFNGWTVQ DGGDTLLGII GLAGDIGLPS |
| 451 | YLYALSARSL FLAYASSDVS LQKISKDTAD SFSKTTEGNE AVVAMTFIKY |
| 501 | VEKDRSLELL SLSEGAPTMF LQRGESFVRL PLETHQALQP GDRLICLTGG |
| 551 | EDILKYFSQL PIEELLKDPL NPLNTENLID SLTMMLNNET EHSADGTLTI |
| 601 | LSFS* |

A predicted signal peptide is highlighted.

The cp7102 nucleotide sequence <SEQ ID 62> is:

| | |
|---|---|
| 1 | ATGAAACATA CCTTTACCAA GCGTGTTCTA TTTTTTTTCT TTTTAGTGAT |
| 51 | TCCCATTCCC CTACTCCTCA ATCTTATGGT CGTAGGTTTT TTCTCATTTT |
| 101 | CTGCCGCTAA AGCAAATTTA GTACAGGTCC TCCATACCCG TGCTACGAAC |
| 151 | TTAAGTATAG AATTCGAAAA AAAACTGACG ATACACAAGC TTTTCCTCGA |
| 201 | TAGACTTGCC AACACATTAG CCTTAAAATC CTATGCATCT CCTTCTGCAG |
| 251 | AGCCCTATGC ACAGGCATAC AATGAGATGA TGGCACTCTC CAATACAGAC |
| 301 | TTTTCCTTAT GCCTTATAGA TCCCTTTGAT GGATCTGTAA GGACGAAAAA |
| 351 | TCCTGGAGAC CCTTTCATTC GCTATCTAAA ACAGCATCCT GAAATGAAGA |
| 401 | AAAAGCTATC CGCAGCTGTA GGGAAAGCCT TTTTATTGAC CATTCCAGGT |
| 451 | AAACCACTTT TACATTATCT TATTCTAGTT GAAGATGTCG CATCTTGGGA |
| 501 | TTCTACAACG ACTTCAGGAC TGCTTGTAAG TTTCTATCCC ATGTCTTTTT |
| 551 | TACAGAAAGA TTTATTCCAA TCCTTACACA TCACCAAAGG AAATATCTGC |
| 601 | CTTGTAAATA AGTATGGCGA GGTCCTCTTC TGTGCTCAGG ACAGTGAATC |
| 651 | TTCTTTTGTA TTTTCTCTAG ATCTCCCTAA TTTACCGCAA TTCCAAGCAA |
| 701 | GAAGCCCCTC TGCCATAGAA ATTGAGAAAG CTTCTGGAAT TCTTGGTGGG |
| 751 | GAGAACCTAA TCACAGTGAG TATCAACAAG AAACGCTACC TAGGATTGGT |
| 801 | ACTGAATAAA ATTCCTATCC AAGGGACCTA CACTCTATCT TTAGTTCCAG |
| 851 | TTTCTGATCT CATCCAATCC GCCTTGAAAG TTCCTCTCAA TATTTGTTTT |
| 901 | TTCTATGTAC TTGCTTTCCT CCTCATGTGG TGGATTTTCT CTAAGATCAA |
| 951 | CACCAAACTT AACAAGCCTC TTCAAGAACT GACCTTCTGT ATGGAAGCTG |
| 1001 | CCTGGCGAGG AAACCATAAC GTGAGGTTTG AACCCCAGCC TTACGGTTAT |
| 1051 | GAATTCAATG AACTAGGAAA TATTTTCAAT TGCACTCTCC TACTCTTATT |
| 1101 | GAATTCCATT GAGAAAGCAG ATATCGATTA CCATTCAGGC GAAAAATTAC |
| 1151 | AAAAAGAATT AGGGATTTTA TCTTCACTAC AAAGTGCGTT ACTAAGTCCG |
| 1201 | GATTTCCCTA CGTTCCCTAA AGTTACCTTT AGTTCCCAAC ATCTCCGGAG |
| 1251 | AAGGCAACTT TCCGGTCATT TTAATGGTTG GACAGTTCAA GATGGTGGCG |
| 1301 | ATACCCTTTT AGGGATCATA GGGCTCGCTG GCGATATTGG TCTTCCTTCC |
| 1351 | TATCTCTATG CTTTATCCGC ACGGAGTCTT TTTCTTGCCT ATGCTTCCTC |
| 1401 | GGACGTTTCG TTACAAAAAA TCAGCAAGGA TACTGCCGAC AGCTTCTCAA |
| 1451 | AAACAACAGA AGGCAATGAG GCTGTAGTTG CTATGACTTT CATTAAATAT |
| 1501 | GTAGAAAAAG ATCGATCTCT AGAGCTCCTC TCGTTAAGCG AGGGAGCTCC |
| 1551 | TACCATGTTT CTACAACGAG GAGAATCTTT CGTACGTCTC CCCTTAGAGA |
| 1601 | CTCACCAAGC TCTACAGCCT GGAGATCGGT TGATCTGCCT CACTGGAGGA |
| 1651 | GAAGACATCC TCAAGTACTT TTCTCAGCTT CCTATTGAAG AGCTCTTAAA |
| 1701 | AGATCCTTTA AACCCTCTAA ATACAGAGAA TCTTATTGAT TCTCTAACCA |
| 1751 | TGATGTTAAA CAACGAAACC GAACATTCTG CAGATGGAAC TCTGACCATC |
| 1801 | CTTTCATTTT CATAA |

The PSORT algorithm predicts an inner membrane location (0.338).

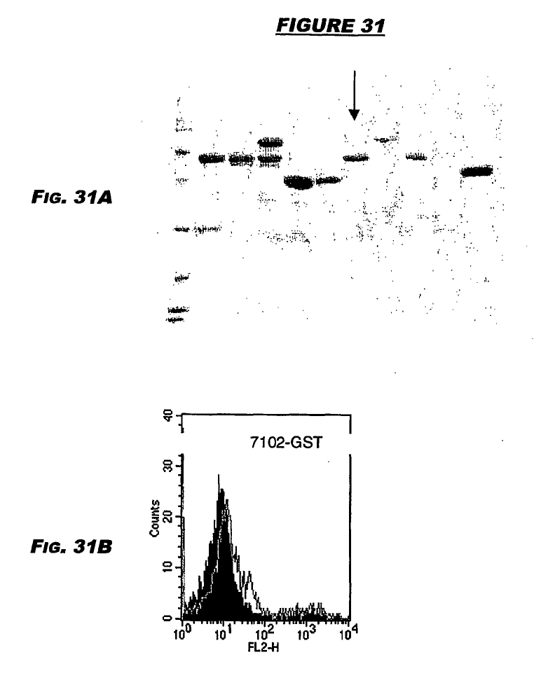

The protein was expressed in E. coli and purified as a his-tag product and as a GST-fusion product. The purified GST-fusion product is shown in FIG. 31A. The recombinant GST-fusion protein was used to immunize mice, whose sera were used in a Western blot and for FACS analysis (FIG. 31B).

These experiments show that cp7102 is a surface-exposed and immunoaccessible protein, and that it is a useful immunogen. These properties are not evident from the sequence alone.

### Example 32

The following C. pneumoniae protein (PID 4377106) was expressed <SEQ ID 63; cp7106>:

| | |
|---|---|
| 1 | MKDLGTLGGT SSTAKTVSPD GKVIMGRSQI ADGSWHAFMC HTDFSSNNVL |
| 51 | FDLDNTYKTL RENGRQLNSI FNLQNMMLQR ASDHEFTEFG RSNIALGAGL |
| 101 | YVNALQNLPS NLAAQYFGIA YKIRPKYRLG VFLDHNFSSH VPNNFNVSHN |
| 151 | RLWMGAFIGW QDSDALGSSV KVSFGYGKQK ATITREQLEN TEAGSGESHF |
| 201 | EGVAAQIEGR YGKSLGGHVR VQPFLGLQFV HITRKEYTEN AVQFPVHYDP |
| 251 | IDYSTGVVYL GIGSHIALVD SLHVGTRMGM EQNFAAHTDR FSGSIASIGN |
| 301 | FVFEKLDVTH TRAFAEMRVN YELPYLQSLN LILRVNQQPL QGVMGFSSDL |
| 351 | RYALGF* |

The cp7106 nucleotide sequence <SEQ ID 64> is:

| | |
|---|---|
| 1 | ATGAAAGATT TGGGGACTCT TGGGGGTACC TCTTCTACAG CAAAAACAGT |
| 51 | GTCCCCAGAT GGTAAAGTGA TCATGGGTAG ATCACAAATT GCTGATGGCA |
| 101 | GTTGGCACGC ATTTATGTGT CATACGGATT TCTCCTCTAA TAATGTACTC |
| 151 | TTTGATCTCG ATAATACGTA TAAAACTCTA AGAGAAAATG GCCGTCAGCT |
| 201 | AAATTCCATA TTCAACCTAC AAAATATGAT GTTACAGAGA GCCTCAGATC |
| 251 | ATGAGTTCAC AGAGTTTGGA AGGAGTAACA TCGCTCTTGG TGCCGGGCTT |
| 301 | TATGTGAATG CCTTGCAGAA TCTCCCTAGC AATTTAGCAG CACAATATTT |
| 351 | TGGAATCGCA TACAAAATAC GTCCTAAATA TCGTTTGGGG GTGTTTTTGG |
| 401 | ACCATAATTT CAGCTCCCAC GTTCCTAATA ATTTTAACGT AAGCCACAAT |
| 451 | AGACTCTGGA TGGGAGCCTT TATTGGATGG CAGGATTCTG ATGCTCTAGG |
| 501 | ATCTAGTGTC AAGGTGTCTT TCGGATATGG AAAACAAAAA GCCACGATTA |
| 551 | CAAGAGAGCA ATTAGAGAAT ACAGAAGCCG GGAGTGGGGA GAGCCATTTT |
| 601 | GAAGGGGTCG CTGCTCAGAT AGAAGGGCGG TATGGTAAGA GCCTCGGAGG |
| 651 | ACATGTCAGG GTCCAGCCTT TCCTAGGACT GCAGTTTGTC CACATTACAA |
| 701 | GGAAAGAATA TACCGAAAAT GCAGTGCAAT TTCCTGTACA CTATGATCCT |
| 751 | ATAGACTATT CTACAGGTGT AGTGTATTTA GGAATTGGAT CTCATATTGC |
| 801 | ACTTGTAGAT TCTTTACATG TAGGCACACG CATGGGAATG GAGCAAAACT |
| 851 | TTGCAGCCCA TACGGACAGG TTCTCAGGAT CTATAGCGTC TATTGGAAAC |
| 901 | TTTGTGTTTG AAAAGCTTGA TGTGACTCAC ACAAGGGCAT TTGCGGAAAT |
| 951 | GCGTGTCAAC TATGAGCTTC CCTATCTACA GTCTCTGAAT CTTATTCTAC |
| 1001 | GAGTTAATCA ACAGCCTCTA CAAGGGGTTA TGGGATTTTC CAGTGATCTT |
| 1051 | AGGTATGCCT TAGGATTCTA A |

The PSORT algorithm predicts a cytoplasmic location (0.224).

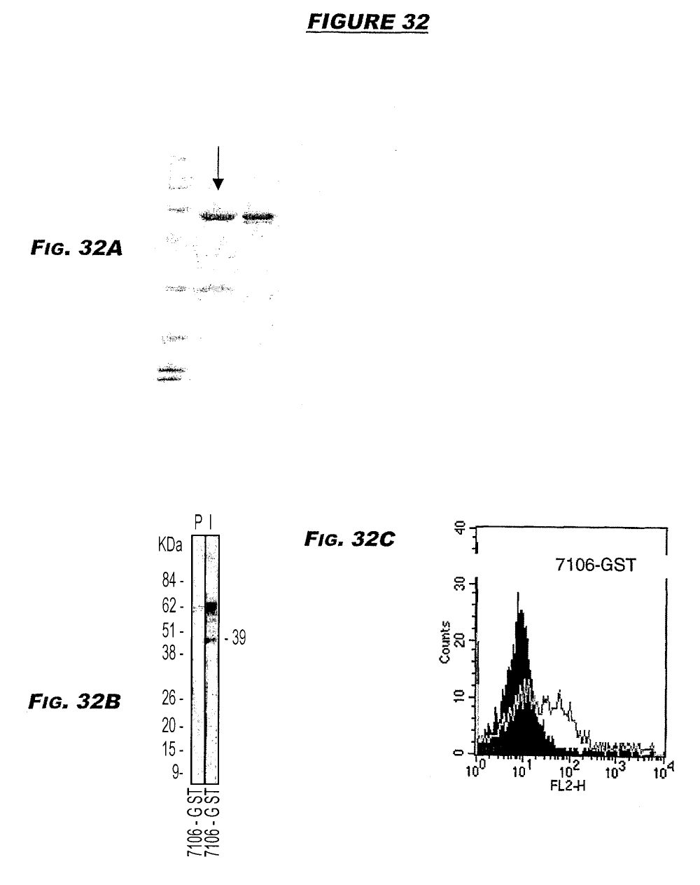

The protein was expressed in E. coli and purified as a his-tag product and as a GST-fusion product. The purified GST-fusion product is shown in FIG. 32A. The recombinant GST-fusion protein was used to immunize mice, whose sera were used in a Western blot (FIG. 32B) and for FACS analysis (FIG. 32C).

This protein also showed very good cross-reactivity with human sera, including sera from patients with pneumonitis.

These experiments show that cp7106 is a surface-exposed and immunoaccessible protein, and that it is a useful immunogen. These properties are not evident from the sequence alone.

### Example 33

The following C. pneumoniae protein (PID 4377228) was expressed <SEQ ID 65; cp7228>:

| | |
|---|---|
| 1 | MTAVLILTSF PSEESARSLA RHLITERLAS CVHVFPKGTS TYLWEGKLCE |
| 51 | SEEHHIQIKS IDIRFSEICL AIQEFSGYEV PEVLLFPIEN GDPRYLNWLT |
| 101 | ILSYPEKPPL SD* |

The cp7228 nucleotide sequence <SEQ ID 66> is:

| | |
|---|---|
| 1 | ATGACTGCTG TTCTTATTCT TACATCTTTC CCTTCGGAGG AAAGTGCTCG |
| 51 | CTCCTTAGCT AGACATCTGA TTACAGAGCG TCTTGCTTCC TGTGTGCATG |
| 101 | TATTCCCTAA AGGCACATCG ACATATCTAT GGGAAGGCAA GCTATGTGAG |
| 151 | TCTGAAGAAC ATCATATACA AATCAAATCG ATAGACATAC GCTTCTCGGA |
| 201 | AATTTGTCTT GCTATTCAGG AGTTCTCTGG CTATGAGGTT CCTGAAGTCT |
| 251 | TACTATTTCC TATTGAAAAT GGGGATCCGA GGTACTTGAA TTGGTTAACG |
| 301 | ATTCTCAGCT ATCCAGAGAA GCCTCCGCTT TCAGATTAG |

The PSORT algorithm predicts an inner membrane location (0.040).

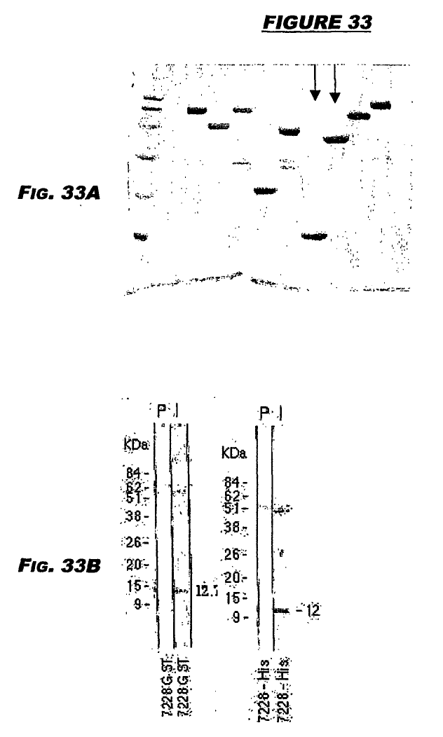

The protein was expressed in E. coli and purified as a his-tag product and as a GST-fusion product, as shown in FIG. 33A (his-tag=left-hand arrow, GST=right-hand arrow). The proteins were used to immunize mice, whose sera were used in a Western blot (FIG. 33B) and FACS analysis.

These experiments show that cp7228 is a surface-exposed and immunoaccessible protein, and that it is a useful immunogen. These properties are not evident from the sequence alone.

### Example 34

The following C. pneumoniae protein (PID 4377170) was expressed <SEQ ID 67; cp7170>:

| | |
|---|---|
| 1 | MNSKMLKHLR LATLSFSMFF GIVSSPAVYA LGAGNPAAPV LPGVNPEQTG |
| 51 | WCAFQLCNSY DLFAALAGSL KFGFYGDYVF SESAHITNVP VITSVTTSGT |
| 101 | GTTPTITSTT KNVDFDLNNS SISSSCVFAT IALQETSPAA IPLLDIAFTA |
| 151 | RVGGLKQYYR LPLNAYRDFT SNPLNAESEV TDGLIEVQSD YGIVWGLSLQ |
| 201 | KVLWKDGVSF VGVSADYRHG SSPINYIIVY NKANPEIYFD ATDGNLSYKE |
| 251 | WSASIGISTY LNDYVLPYAS VSIGNTSRKA PSDSFTELEK QFTNFKFKIR |
| 301 | KITNFDRVNF VFGTTCCISN NFYYSVEGRW GYQRAINITS GLQF* |

A predicted signal peptide is highlighted.

The cp7170 nucleotide sequence <SEQ ID 68> is:

```
      1 ATGAATAGCA AGATGCTAAA ACATTTACGT TTAGCAACCC TTTCCTTCTC
    51 TATGTTCTTC GGGATTGTAT CTTCTCCCGC AGTATATGCC CTAGGGGCTG
  101 GAAACCCTGC AGCTCCAGTA CTCCCAGGTG TGAATCCTGA GCAAACGGGA
  151 TGGTGTGCCT TCCAACTTTG TAATAGTTAC GATCTTTTTG CTGCTCTTGC
  201 AGGAAGCCTC AAATTTGGGT TCTATGGAGA TTATGTCTTC TCAGAAAGTG
  251 CCCATATTAC CAATGTCCCT GTCATTACCT CCGTTACGAC TTCAGGCACA
  301 GGAACAACGC CAACCATTAC CTCTACAACT AAAAACGTAG ACTTTGATCT
  351 TAACAACAGC TCCATCAGCT CGAGCTGTGT TTTTGCAACC ATAGCTCTAC
  401 AGGAAACATC CCCAGCTGCC ATTCCCCTTT TAGATATAGC CTTCACTGCA
  451 CGTGTCGGAG GACTTAAGCA GTACTACCGC CTCCCTCTCA ATGCTTACAG
  501 AGACTTCACT TCAAATCCTT TAAATGCAGA ATCTGAAGTT ACAGATGGTC
  551 TCATTGAAGT CCAGTCAGAC TATGGAATTG TCTGGGGTCT GAGTTTACAA
  601 AAAGTATTGT GGAAAGATGG AGTGTCTTTT GTAGGGGTGA GCGCTGACTA
  651 CCGTCACGGT TCCAGTCCCA TCAACTATAT CATCGTTTAC AACAAGGCCA
  701 ACCCCGAGAT CTATTTCGAT GCTACTGATG GAAACCTAAG CTATAAAGAA
  751 TGGTCTGCAA GCATCGGCAT CTCTACGTAT CTTAATGACT ATGTGCTTCC
  801 CTATGCATCC GTATCTATAG GAAATACTTC AAGAAAAGCT CCTTCTGATA
  851 GCTTCACAGA ACTCGAAAAG CAATTTACGA ATTTTAAATT TAAAATTCGT
  901 AAAATCACAA ACTTCGACAG AGTAAACTTC TGCTTCGGAA CTACCTGCTG
  951 CATCTCAAAT AACTTCTACT ATAGTGTAGA AGGCCGTTGG GGATATCAGC
1001 GTGCTATCAA CATTACGTCA GGTCTGCAGT TTTAG
```

The PSORT algorithm predicts a bacterial outer membrane location (0.936).

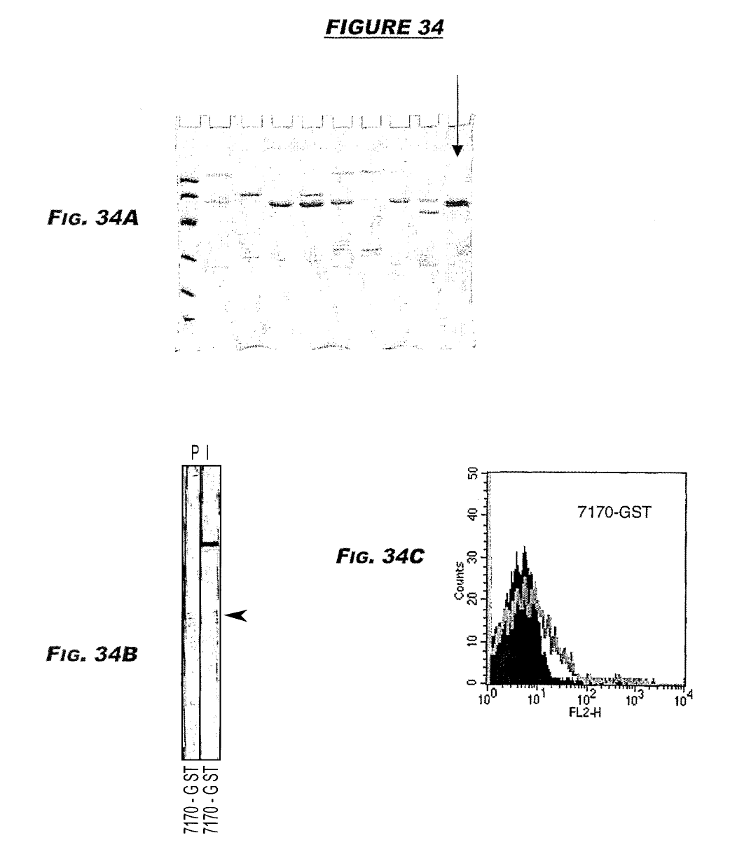

The protein was expressed in E. coli and purified as a his-tag product and as a GST-fusion product. The purified GST-fusion product is shown in FIG. 34A. The GST-fusion protein was used to immunize mice, whose sera were used in a Western blot (34B) and for FACS analysis (34C).

The cp7170 protein was also identified in the 2D-PAGE experiment (Cpn0854).

These experiments show that cp7170 is a surface-exposed and immunoaccessible protein, and that it is a useful immunogen. These properties are not evident from the sequence alone.

### Example 35

The following C. pneumoniae protein (PID 4377072) was expressed <SEQ ID 69; cp7072>:

```
    1 MDIKKLFCLF LCSSLIAMSP IYGKTGDYEK LTLTGINIID RNGLSETICS
  51 KEKLKKYTKV DFLAPQPYQK VMRMYKNKRG DNVSCLTAYH TNGQIKQYLE
101 CLNNRAYGRY REWHVNGNIK IQAEVIGGIA DLHPSAESGW LFDQTTFAYN
151 DEGILEAAIV YEKGLLEGSS VYYHTNGNIW KECPYHKGVP QGKFLTYTSS
201 GKLLKEQNYQ QGKRHGLSIR YSEDSEEDVL AWEEYHEGRL LKAEYLDPQT
251 HETYATIHEG NGIQAIYGKY AVIETRAFYR GEPYGKVTRF DNSGTQIVQT
301 YNLLQGAKHG EEFFFYPETG KPKLLLNWHE GILNGIVKTW YPGGTLESCK
351 ELVNNKKSGL LTIYYPEGQI MATEEYDNDL LIKGEYFRPG DRHPYSKIDR
401 GCGTAVFFSS AGTITKKIPY QDGKPLLN*
```

A predicted signal peptide is highlighted.

The cp7072 nucleotide sequence <SEQ ID 70> is:

```
      1 ATGGATATAA AAAAACTCTT TTGCTTATTT CTATGTTCTT CTCTAATTGC
    51 CATGAGTCCC ATTTATGGGA AAACAGGTGA CTATGAGAAA CTCACCCTTA
  101 CAGGGATCAA TATCATTGAT AGAAACGGCC TGTCAGAAAC TATTTGCTCT
  151 AAAGAGAAGC TAAAGAAATA CACCAAGGTA GACTTTCTTG CTCCCCAGCC
  201 CTATCAAAAG GTCATGAGGA TGTATAAAAA CAAACGCGGA GATAACGTTT
  251 CTTGTTTAAC AGCCTATCAC ACTAACGGGC AAATTAAGCA GTACCTGGAG
  301 TGTCTCAATA ATCGTGCTTA TGGAAGATAT CGTGAATGGC ACGTCAACGG
  401 CCTCAGCAGA GTCTGGCTGG CTATTTGATC AAACTACATT TGCCTATAAT
  451 GATGAAGGTA TCTTAGAAGC CGCTATCGTC TATGAAAAAG GGCTGCTCGA
  501 AGGATCTTCG GTGTATTACC ATACTAATGG GAATATTTGG AAAGAGTGTC
  551 CCTATCATAA GGGAGTTCCT CAAGGTAAAT TCCTGACATA CACATCTTCG
  601 GGGAAACTGC TCAAAGAACA GAATTACCAA CAAGGCAAAA GACACGGTCT
  651 TTCGATTCGC TACAGCGAAG ATTCCGAAGA AGATGTTTTA GCCTGGGAAG
  701 AATATCATGA GGGACGACTC CTAAAAGCAG AGTACTTAGA TCCTCAAACT
  751 CACGAAATCT ATGCGACTAT ACACGAAGGG AACGGCATTC AAGCAATCTA
  801 CGGCAAGTAT GCCGTTATAG AAACTAGGGC ATTTTACCGA GGGGAACCTT
  851 ATGGAAAAGT TACCAGATTC GACAACTCCG GAACACAGAT TGTCCAAACG
  901 TATAACCTTT TGCAAGGCGC GAAGCACGGA GAAGAATTTT TCTTTTATCC
  951 TGAGACAGGG AAACCCAAGC TGCTTCTTAA TTGGCATGAA GGAATTTTAA
1001 ATGGGATAGT AAAAACTTGG TATCCCGGAG GAACCTTAGA AAGTTGTAAA
1051 GAACTCGTAA ATAACAAAAA ATCCGGGTTA CTGACCATTT ACTACCCTGA
1101 AGGACAGATC ATGGCGACCG AAGAGTATGA TAATGATCTT CTAATTAAAG
1151 GAGAGTACTT CCGCCCTGGA GACCGTCATC CCTACTCTAA AATAGATCGT
1201 GGTTGTGGGA CTGCAGTATT TTTCTCGTCG GCGGGAACTA TTACTAAAAA
1251 AATCCCCTAT CAGGACGGCA AACCTTTGCT CAACTAG
```

The PSORT algorithm predicts a periplasmic location (0.688).

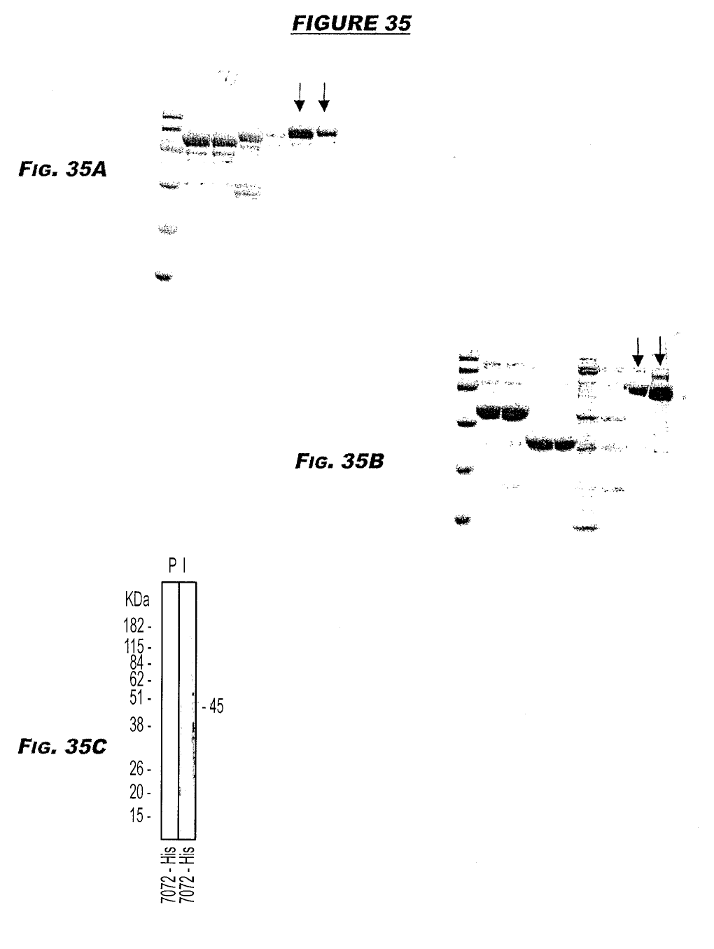

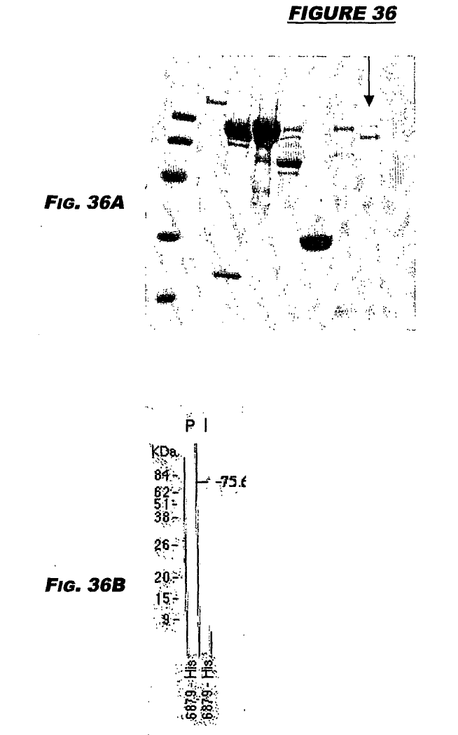

The protein was expressed in E. coli and purified as a his-tag product (FIG. 35A) and as a GST-fusion product (FIG. 35B). The recombinant his-tag protein was used to immunize mice, whose sera were used in a Western blot (FIG. 35C) and for FACS analysis.

These experiments show that cp7072 is a useful immunogen. These properties are not evident from the sequence alone.

### Example 36

The following C. pneumoniae protein (PID 4376879) was expressed <SEQ ID 71; cp6879>:

```
    1 MATPAQKSPT FQDPSFVREL GSNHPVFSPL TLEERGEMAI ARVQQCGWNH
  51 TIVKVSLIIL ALLTILGGGL LVGLLPAVPM FIGTGLIALG AVIFALALIL
101 CLYDSQGLPE ELPPVPEPQQ IQIEDLRNET REVLEGTLLE VLLKDRDAKD
151 PAVPQVVVDC EKRLGMLDRK LRREEEILYR STAHLKDEER YEFLLELLEM
201 RSLVADRLEF NRRSYERFVQ GIMTVRSEEG EKEISRLQDL ISLQQQTVQD
251 LRSRIDDEQK RCWTALQRIN QSQKDIQRAH DREASQRACE GTEMDCAERQ
301 QLEKDLRRQL KSMQEWIEMR GTIHQQEKAW RKQNAKLERL QEDLRLTGIA
351 FDEQSLFYRE YKEKYLSQKL DMQKILQEVN AEKSEKACLE SLVHDYEKQL
401 EQKDANLKKA AAVWEEELGK QQQEDYEQTQ EIRRLSTFIL EYQDSLREAE
451 KVEKDFQELQ QRYSRLQEEK QVKEKILEES MNHFADLFEK AQKENMAYKK
501 KLADLEGAAA PTEIGEDDDW VLTDSASLSQ KKIRELVEEN QELLKALAFK
551 SNELTQLVAD AVEAEKEISK LREHIEEQKE GLRALDKMHA QAIKDCEAAQ
601 RKCCDLESLL SPVREDAGMR FELEVELQRL QEENAQLRAE VERLEQEQFQ
651 G*
```

The cp6879 nucleotide sequence <SEQ ID 72> is:

```
      1 ATGGCAACAC CCGCTCAAAA ATCCCCTACA TTTCAAGATC CTAGTTTTGT
    51 AAGAGAGCTA GGCAGTAACC ACCCTGTCTT TTCCCCGCTA ACGCTTGAGG
  101 AAAGAGGGGA GATGGCAATA GCTCGAGTCC AGCAGTGTGG ATGGAATCAT
  151 ACAATTGTTA AGGTAAGTCT TATTATTCTT GCTCTTCTTA CTATTTTAGG
  201 GGGAGGATTA CTCGTAGGAT TGCTGCCAGC AGTTCCTATG TTTATTGGAA
  251 CAGGTCTGAT TGCTTTGGGA GCCGTTATAT TTGCTTTGGC TTTGATTTTA
  301 TGTCTTTATG ATTCTCAGGG CCTTCCTGAG GAACTCCCTC CGGTTCCTGA
  351 ACCACAACAA ATTCAGATTG AAGATTTAAG AAACGAGACC AGAGAAGTTC
  401 TTGAAGGGAC TCTTTTAGAG GTTCTCTTAA AGGATAGAGA CGCTAAGGAC
  451 CCTGCGGTGC CCCAGGTGGT TGTAGACTGT GAAAAGCGTC TTGGAATGTT
  501 GGATCGTAAG CTGCGACGTG AAGAGGAGAT TCTGTATCGC TCGACGGCCC
  551 ATCTTAAAGA CGAGGAAAGG TATGAGTTCT TGCTGGAGCT CTTGGAAATG
  501 CGTAGTCTGG TTGCCGATCG GCTAGAATTT AACCGTAGAA GTTATGAGCG
  651 ATTTGTTCAA GGAATTATGA CAGTTAGATC AGAGGAGGGG GAAAAAGAGA
  701 TTTCTCGTCT ACAAGATCTA ATCAGTTTGC AGCAGCAGAC GGTGCAAGAT
  751 TTAAGGAGTC GGATCGATGA CGAGCAGAAG AGATGCTGGA CGGCTTTACA
  801 ACGTATTAAC CAATCTCAGA AGGATATACA ACGGGCTCAT GATCGCGAGG
  851 CTTCGCAGCG TGCCTGTGAG GGCACAGAGA TGGATTGTGC AGAACGCCAG
  901 CAACTGGAGA AGGATTTAAG GAGACAGCTG AAATCTATGC AGGAGTGGAT
  951 TGAGATGAGG GGCACAATCC ATCAACAAGA GAAGGCTTGG CGTAAGCAGA
1001 ATGCCAAATT AGAAAGATTA CAAGAGGATC TGAGACTTAC TGGGATTGCT
1051 TTTGACGAAC AATCTCTGTT CTATCGCGAA TATAAAGAGA AATATCTGAG
1101 TCAGAAACTA GATATGCAAA AGATTTTACA GGAAGTCAAC GCAGAGAAAA
1151 GTGAGAAGGC TTGCTTAGAG AGTCTGGTCC ATGACTATGA GAAGCAGCTC
1201 GAACAAAAAG ATGCTAATCT GAAGAAAGCA GCAGCTGTTT GGGAAGAAGA
1251 ATTAGGGAAG CAGCAACAGG AAGACTACGA ACAAACCCAA GAAATTAGAC
1301 GTCTGAGTAC ATTCATTCTT GAGTACCAGG ACAGTCTGCG TGAGGCAGAA
1351 AAAGTTGAGA AAGATTTCCA AGAGCTACAA CAAAGGTATA GCCGTCTTCA
1401 AGAGGAGAAA CAGGTAAAAG AAAAAATCTT AGAAGAAAGT ATGAATCATT
1451 TTGCCGATCT CTTTGAGAAG GCTCAAAAGG AAAACATGGC CTACAAGAAG
1501 AAGTTAGCGG ATTTAGAGGG TGCCGCTGCT CCTACTGAGA TCGGTGAGGA
1551 CGATGACTGG GTACTCACAG ATTCTGCTTC TCTCAGCCAG AAGAAGATCC
1601 GCGAACTCGT GGAAGAGAAT CAAGAACTCC TGAAAGCACT TGCATTTAAA
1651 TCTAACGAAT TGACTCAACT GGTTGCCGAT GCTGTAGAAG CTGAAAAAGA
1701 AATCAGCAAG CTTCGAGAAC ACATAGAAGA GCAGAAAGAA GGATTACGAG
1751 CTCTTGATAA GATGCATGCA CAAGCGATCA AAGATTGCGA AGCTGCTCAG
1801 AGAAAATGCT GTGACCTTGA GAGCCTTCTC TCTCCTGTTC GAGAAGATGC
1851 TGGAATGAGA TTTGAGCTAG AGGTCGAGCT TCAAAGATTG CAAGAAGAAA
1901 ATGCACAGCT TAGAGCGGAG GTTGAAAGAC TAGAGCAAGA GCAATTTCAA
1951 GGATAA
```

The PSORT algorithm predicts an inner membrane location (0.646).

The protein was expressed in E. coli and purified as a his-tag product and as a GST-fusion product. The purified GST-fusion product is shown in FIG. 36A. The recombinant GST-fusion protein was used to immunize mice, whose sera were used in a Western blot (FIG. 36B) and for FACS analysis.

These experiments show that cp6879 is useful immunogen. These properties are not evident from the sequence alone.

### Example 37

The following C. pneumoniae protein (PID 4376767) was expressed <SEQ ID 73; cp6767>:

```
    1 MIKQIGRFFR AFIFIMPLSL TSCESKIDRN RIWIVGTNAT YPPFEYVDAQ
  51 GEVVGFDIDL AKAISEKLGK QLEVREFAFD ALILNLKKHR IDAILAGMSI
101 TPSRQKEIAL LPYYGDEVQE LMVVSKRSLE TPVLPLTQYS SVAVQTGTFQ
151 EHYLLSQPGI CVRSFDSTLE VIMEVRYGKS PVAVLEPSVG RVVLKDFPNL
201 VATRLELPPE CWVLGCGLGV AKDRPEEIQT IQQAITDLKS EGVIQSLTKK
251 WQLSEVAYE*
```

The cp6767 nucleotide sequence <SEQ ID 74> is:

```
    1 ATGATAAAAC AAATAGGCCG TTTTTTTAGA GCATTTATTT TTATAATGCC
  51 TTTATCTTTA ACAAGTTGTG AGTCTAAAAT CGATCGAAAT CGCATCTGGA
101 TTGTAGGTAC GAATGCTACA TATCCTCCTT TTGAGTATGT GGATGCTCAG
151 GGGGAAGTTG TAGGTTTCGA TATAGATTTG GCAAAGGCAA TTAGTGAAAA
201 ACTTGGCAAG CAATTGGAAG TTAGAGAATT CGCTTTCGAT GCTTTAATTT
251 TAAATTTAAA AAAACATCGT ATCGATGCAA TTTTAGCAGG AATGTCCATT
301 ACTCCTTCGC GTCAGAAGGA AATCGCCCTG CTTCCCTATT ATGGCGATGA
351 GGTTCAAGAG CTGATGGTGG TTTCTAAGCG GTCTTTAGAG ACCCCTGTGC
401 TTCCCCTAAC ACAGTATTCT TCTGTTGCTG TTCAGACAGG AACGTTTCAG
451 GAGCATTATC TTTTATCTCA GCCCGGAATT TGTGTCCGTT CTTTTGATAG
501 CACCTTGGAG GTGATTATGG AAGTTCGTTA TGGGAAATCT CCGGTTGCCG
551 TTCTAGAACC CTCGGTAGGA CGTGTCGTTC TTAAAGACTT CCCTAATCTT
501 GTTGCAACAA GATTAGAGCT CCCTCCTGAA TGTTGGGTGT TGGGCTGTGG
551 TCTCGGCGTA GCTAAAGATC GTCCTGAAGA AATACAAACG ATTCAACAAG
701 CGATTACAGA TTTAAAGAGC GAAGGGGTGA TTCAATCTTT AACCAAGAAA
751 TGGCAACTTT CTGAAGTTGC TTACGAATAG
```

The PSORT algorithm predicts an inner membrane location (0.083).

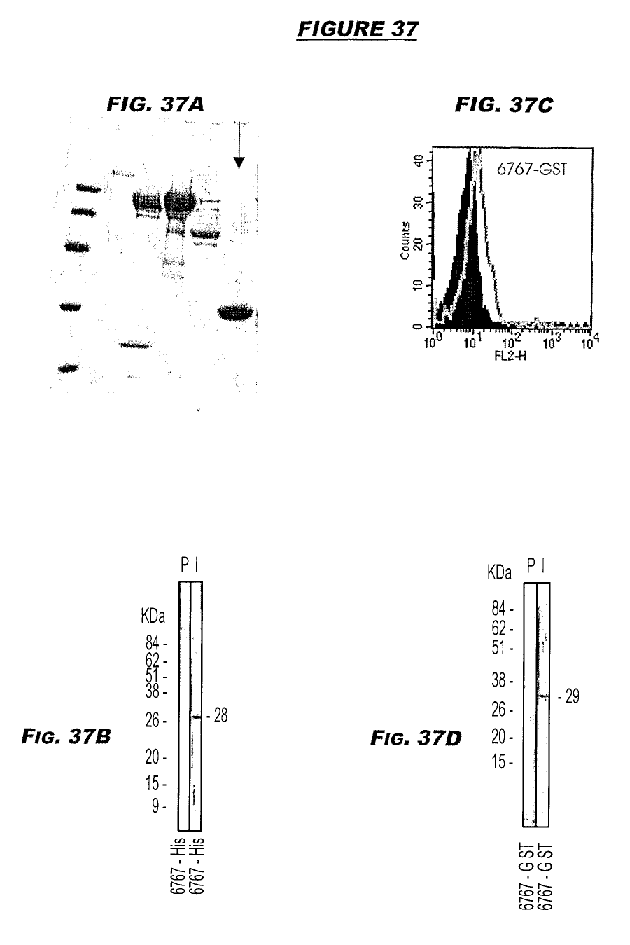

The protein was expressed in E. coli and purified as a his-tag product and as a GST-fusion product. The purified his-tag product is shown in FIG. 37A. The recombinant his-tag protein was used to immunize mice, whose sera were used in a Western blot (FIG. 37B) and for FACS analysis (FIG. 37C). The GST-fusion was also used in a Western blot (FIG. 37D).

The cp6767 protein was also identified in the 2D-PAGE experiment and showed good cross-reactivity with human sera, including sera from patients with pneumonitis.

These experiments show that cp6767 is a useful immunogen. These properties are not evident from the sequence alone.

### Example 38

The following C. pneumoniae protein (PID 4376717) was expressed <SEQ ID 75; cp6717>:

```
    1 MMSRLRFRLA ALGIFFILLV PNSVSAKTIV ASDKEKVGVL VYDNSVEAFQ
  51 QILDCIDHAN FYVELCPCMT GGRTLKEMVD HLEARMDLVP ELCSYIIIQP
101 TFTDAEDQKL LKALKERHPN RFFYVFTGCP PSTSILAPNV IEMHTKLSII
151 DGKYCILGGT NFEEFMCTPG DEVPEKVDNP RLFVSGVRRP LAFRDQDIML
201 RSTAFGLQLR EEYHKQFAMW DYYAHHMWFI DNPEQFAGAC PPLTLEQAEE
251 TVFPGFDKHE DLVLVDSSKI RIVLGGPHDK QPNPVTQEYL KLIQGARSSV
301 KLAHMYFIPK DELLNALVDV SHNHGVHLSL ITNGCHELSP AITGPYAWGN
351 RINYFALLYG KRYPLWKKWF CEKLKPYERV SIYEFAIWET QLHKKCMIID
401 DEIFVIGSYN FGKKSDAFDY ESIVVIESPE VAAKANKVFN KDIGLSIPVS
451 HGDIFSWYFH SVHHTLGHLQ LTYMPA*
```

A predicted signal peptide is highlighted.

The cp6717 nucleotide sequence <SEQ ID 76> is:

```
      1 ATGATGAGTC GGTTGCGTTT TCGCTTGGCA GCTCTTGGAA TATTTTTTAT
    51 TTTGCTGGTT CCTAATTCTG TTTCAGCAAA GACAATCGTA GCTTCAGACA
  101 AGGAGAAGGT TGGAGTTCTT GTTTATGACA ATAGTGTAGA GGCCTTTCAA
  151 CAGATATTGG ATTGCATAGA TCATGCAAAT TTTTATGTAG AACTGTGTCC
  201 CTGCATGACA GGAGGCCGAA CGCTTAAAGA GATGGTAGAT CACCTCGAGG
  251 CTCGTATGGA TCTGGTTCCA GAGCTCTGTA GCTATATCAT TATCCAACCC
  301 ACGTTTACCG ATGCTGAAGA CCAAAAATTA CTCAAAGCTC TCAAAGAACG
  351 TCATCCCAAC CGGTTTTTCT ACGTTTTTAC AGGGTGCCCA CCCTCAACAA
  401 GCATCCTCGC TCCTAATGTC ATTGAAATGC ATATCAAACT TTCTATCATC
  451 GATGGGAAAT ATTGTATTTT AGGTGGTACC AATTTTGAAG AGTTTATGTG
  501 CACTCCAGGG GATGAGGTTC CTGAGAAAGT GGATAACCCA CGTTTATTTG
  551 TCAGTGGAGT GCGTCGGCCC CTAGCATTTC GTGATCAGGA TATCATGTTG
  601 CGTTCTACAG CATTCGGTTT GCAGCTCAGA GAAGAATATC ATAAGCAATT
  651 TGCTATGTGG GACTACTATG CACATCATAT GTGGTTCATT GATAATCCTG
  701 AACAGTTTGC AGGCGCCTGT CCTCCACTGA CTTTAGAACA AGCCGAGGAG
  751 ACAGTATTTC CTGGATTTGA CAAACATGAA GATCTTGTTC TTGTCGACTC
  801 TTCCAAGATC AGGATAGTTT TAGGTGGTCC CCACGATAAG CAACCCAATC
  851 CTGTGACTCA AGAATATTTG AAACTTATCC AGGGAGCTAG ATCTTCTGTG
  901 AAGCTTGCTC ACATGTATTT CATCCCTAAG GACGAGCTTT TAAATGCTCT
  951 TGTCGACGTT TCTCATAATC ACGGTGTTCA TCTGAGTTTA ATTACGAACG
1001 GCTGTCATGA ATTAAGTCCT GCAATTACAG GACCCTATGC TTGGGGAAAC
1051 CGTATTAACT ATTTCGCCTT GCTCTATGGG AAACGGTATC CTCTTTGGAA
1101 AAAATGGTTT TGCGAAAAGC TAAAACCTTA TGAGCGGGTT TCTATTTATG
1151 AGTTTGCTAT TTGGGAAACG CAGTTGCACA AGAAGTGTAT GATTATCGAT
1201 GATGAAATTT TTGTGATCGG AAGTTATAAT TTTGGAAAGA AAAGTGATGC
1251 CTTTGATTAC GAAAGTATTG TAGTTATCGA ATCTCCAGAA GTCGCTGCAA
1301 AAGCTAACAA AGTCTTCAAT AAAGATATCG GATTGTCGAT TCCTGTAAGT
1351 CATGGCGACA TTTTCTCTTG GTATTTCCAT TCCGTACACC ACACTTTGGG
1401 ACATTTGCAG CTGACCTATA TGCCAGCCTA G
```

The PSORT algorithm predicts a periplasmic location (0.939).

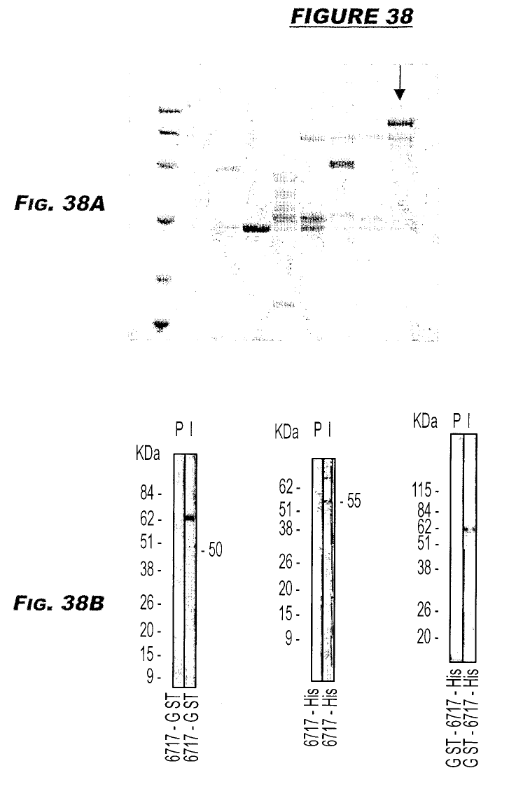

The protein was expressed in E. coli and purified as a GST-fusion (FIG. 38A), as a his-tagged protein, and as a GST/his fusion product. The proteins were used to immunize mice, whose sera were used in a Western blot (FIG. 38B) and for FACS analysis.

These experiments show that cp6717 is a useful immunogen. These properties are not evident from the sequence alone.

### Example 39

The following C. pneumoniae protein (PID 4376577) was expressed <SEQ ID 77; cp6577>:

```
    1 MKKLLFSTFL LVLGSTSAAH ANLGYVNLKR CLEESDLGKK ETEELEAMKQ
  51 QFVKNAEKIE EELTSIYNKL QDEDYMESLS DSASEELRKK FEDLSGEYNA
101 YQSQYYQSTN QSNVKRIQKL IQEVKIAAES VRSKEKLEAI LNEEAVLAIA
151 PGTDKTTEII AILNESFKKQ N*
```

A predicted signal peptide is highlighted.

The cp6577 nucleotide sequence <SEQ ID 78> is:

```
    1 ATGAAAAAAT TATTATTTTC TACATTTCTT CTTGTTTTAG GATCAACAAG
  51 CGCAGCTCAT GCAAATTTAG GCTATGTTAA TTTAAAGCGA TGTCTTGAAG
101 AATCCGATCT AGGTAAAAAG GAAACTGAAG AATTGGAAGC TATGAAACAG
151 CAGTTTGTAA AAAATGCTGA GAAAATAGAA GAAGAACTCA CTTCTATTTA
201 TAATAAGTTG CAAGATGAAG ATTACATGGA AAGCCTATCG GATTCTGCCT
251 CTGAAGAGTT GCGAAAGAAA TTCGAAGATC TTTCAGGAGA GTACAATGCG
301 TACCAGTCTC AGTACTATCA ATCTATCAAT CAAAGTAATG TAAAACGCAT
351 TCAAAAACTC ATTCAAGAAG TAAAAATAGC TGCAGAATCA GTGCGGTCCA
401 AAGAAAAACT AGAAGCTATC CTTAATGAAG AAGCTGTCTT AGCAATAGCA
451 CCTGGGACTG ATAAAACAAC CGAAATTATT GCTATTCTTA ACGAATCTTT
501 CAAAAAACAA AACTAG
```

The PSORT algorithm predicts a periplasmic space location (0.932).

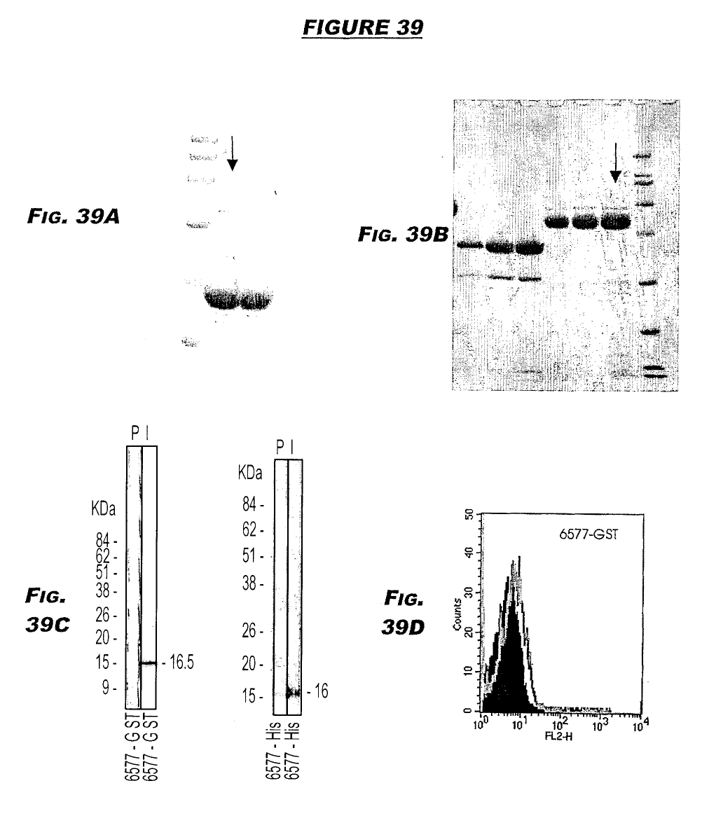

The protein was expressed in E. coli and purified as a his-tag product (FIG. 39A) and as a GST-fusion product (FIG. 39B). The recombinant GST-fusion protein was used to immunize mice, whose sera were used in a Western blot (FIG. 39C) and for FACS analysis.

The cp6577 protein was also identified in the 2D-PAGE experiment.

These experiments show that cp6577 is a useful immunogen. These properties are not evident from the sequence alone.

### Example 40

The following C. pneumoniae protein (PID 4376446) was expressed <SEQ ID 79; cp6446>:

```
    1 MKQPMSLIFS SVCLGLGLGS LSSCNQKPSW NYHNTSTSEE FFVHGNKSVS
  51 QLPHYPSAFR TTQIFSEEHN DPYVVAKTDE ESRKIWREIH KNLKIKGSYI
101 PISTYGSLMH PKSAALTLKT YRPHPIWING YERSFNIDTG KYLKNGSRRR
151 TSHDGPKNRA VLNLIKSSGR RCNAIGLEMT EEDFVIARRR EGVYSLYPVE
201 VCSYPQGNPF VIAYAWIADE SACSKEVLPV KGYYSLVWES VSSSDSLNAF
251 GDSFAEDYLR STFLANGTSI LCVHESYKKV PPQP*
```

A predicted signal peptide is highlighted.

The cp6446 nucleotide sequence <SEQ ID 80> is:

```
    1 ATGAAACAGC CCATGTCTCT TATCTTTTCA AGTGTATGTT TAGGATTAGG
  51 TCTTGGATCT CTTTCCTCCT GTAATCAAAA GCCCTCTTGG AATTATCACA
101 ACACTTCAAC GAGCGAAGAA TTCTTTGTTC ATGGAAATAA GAGTGTTTCG
151 CAACTGCCTC ATTATCCTTC TGCATTTCGT ACGACTCAAA TCTTTTCTGA
201 AGAGCACAAT GATCCTTATG TCGTAGCTAA GACTGATGAA GAGTCTCGTA
251 AAATTTGGAG AGAAATCCAT AAAAATCTCA AAATCAAAGG TTCTTACATT
301 CCCATATCGA CTTATGGAAG TCTGATGCAC CCAAAATCAG CAGCTCTTAC
351 ATTAAAAACG TATCGTCCAC ATCCTATTTG GATAAATGGA TACGAGCGTT
401 CTTTTAATAT AGACACAGGA AAGTACTTAA AAAACGGAAG TCGCCGTAGA
451 ACTTCTCACG ATGGTCCGAA AAATCGAGCT GTACTGAATC TCATTAAATC
501 TTCGGGACGA CGCTGTAATG CTATAGGCCT TGAGATGACA GAAGAAGACT
551 TTGTAATAGC TAGAAGGCGA GAAGGTGTTT ATAGCCTGTA TCCCGTTGAA
601 GTGTGCTCGT ATCCTCAGGG GAATCCTTTT GTCATTGCTT ATGCCTGGAT
651 TGCAGATGAG AGTGCTTGCT CAAAAGAGGT CCTACCTGTA AAAGGGTACT
701 ATTCTTTAGT CTGGGAAAGC GTTTCTTCCT CTGATTCTCT GAATGCTTTT
751 GGAGATTCCT TTGCAGAGGA CTACCTCAGA AGCACGTTTT TAGCAAACGG
801 AACTTCTATA CTCTGTGTTC ATGAAAGCTA TAAGAAAGTT CCTCCTCAGC
851 CCTAA
```

The PSORT algorithm predicts an inner membrane location (0.177).

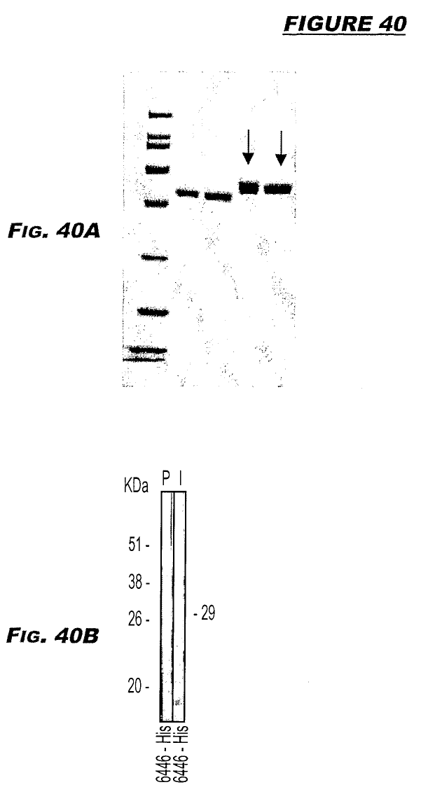

The protein was expressed in E. coli and purified as a his-tag product and a GST-fusion product. The GST-fusion product is shown in FIG. 40A. The recombinant his-tag protein was used to immunize mice, whose sera were used in a Western blot (FIG. 40B) and for FACS analysis.

These experiments show that cp6446 is a useful immunogen. These properties are not evident from the sequence alone.

### Example 41

The following C. pneumoniae protein (PID 4377108) was expressed <SEQ ID 81; cp7108>:

```
    1 MSKKIKVLGH LTLCTLFRGV LCAAALSNIG YASTSQESPY QKSIEDWKGY
  51 TFTDLELLSK EGWSEAHAVS GNGSRIVGAS GAGQGSVTAV IWESHLIKHL
101 GTLGGEASSA EGISKDGEVV VGWSDTREGY THAFVFDGRD MKDLGTLGAT
151 YSVARGVSGD GSIIVGVSAT ARGEDYGWQV GVKWEKGKIK QLKLLPQGLW
201 SEANAISEDG TVIVGRGEIS RNHIVAVKWN KNAVYSLGTL GGSVASAEAI
251 SANGKVIVGW STTNNGETHA FMHKDETMHD LGTLGGGFSV ATGVSADGRA
301 IVGFSAVKTG EIHAFYYAEG EMEDLTTLGG EEARVFDISS EGNDIIGSIK
351 TDAGAERAYL FHTHK*
```

A predicted signal peptide is highlighted.

The cp7108 nucleotide sequence <SEQ ID 82> is:

```
      1 ATGAGTAAGA AGATAAAGGT TCTAGGTCAT TTGACGCTCT GCACTCTGTT
    51 TAGAGGAGTG CTGTGTGCAG CGGCCCTTTC CAACATAGGA TATGCGAGTA
  101 CTTCTCAGGA ATCACCATAT CAGAAGTCTA TAGAAGACTG GAAAGGGTAT
  151 ACCTTTACAG ATCTTGAGTT ACTGAGTAAG GAAGGGTGGT CTGAAGCTCA
  201 TGCAGTTTCT GGAAATGGCA GTAGAATTGT AGGAGCTTCG GGAGCTGGCC
  251 AAGGTAGTGT GACTGCTGTC ATATGGGAAA GTCACCTGAT AAAACATCTC
  301 GGCACTTTAG GTGGCGAGGC TTCATCTGCA GAGGGAATTT CAAAGGATGG
  351 AGAGGTGGTC GTTGGGTGGT CAGATACTAG AGAGGGATAT ACTCATGCCT
  401 TTGTCTTCGA CGGTAGAGAT ATGAAAGATC TCGGTACTCT AGGAGCTACC
  451 TATTCTGTAG CAAGGGGTGT TTCTGGAGAT GGTAGTATCA TCGTAGGAGT
  501 CTCTGCAACT GCTCGTGGAG AGGATTACGG ATGGCAAGTT GGTGTCAAGT
  551 GGGAAAAAGG GAAAATCAAA CAATTGAAGT TGTTGCCTCA AGGTCTCTGG
  601 TCTGAGGCGA ATGCAATCTC TGAGGATGGT ACGGTGATTG TCGGGAGAGG
  651 GGAAATCTCT CGCAATCACA TCGTTGCTGT AAAATGGAAT AAAAATGCTG
  701 TGTATAGTTT GGGGACTCTC GGAGGTAGTG TCGCTTCAGC AGAGGCTATA
  751 TCGGCAAATG GGAAAGTAAT TGTAGGATGG TCCACGACTA ATAATGGTGA
  801 GACTCATGCC TTTATGCACA AAGATGAGAC AATGCACGAT CTCGGCACTC
  851 TAGGAGGAGG TTTTTCTGTC GCAACTGGAG TTTCTGCTGA TGGGAGAGCC
  901 ATCGTAGGAT TTTCAGCAGT GAAGACCGGA GAAATTCATG CTTTTTACTA
  951 TGCAGAAGGA GAAATGGAGG ATTTAACAAC TTTGGGAGGG GAAGAAGCTC
1001 GAGTGTTCGA CATATCTAGC GAAGGAAACG ATATCATTGG CTCTATAAAA
1051 ACTGACGCTG GAGCTGAACG CGCCTATCTG TTCCATATAC ATAAATAA
```

The PSORT algorithm predicts an outer membrane location (0.921).

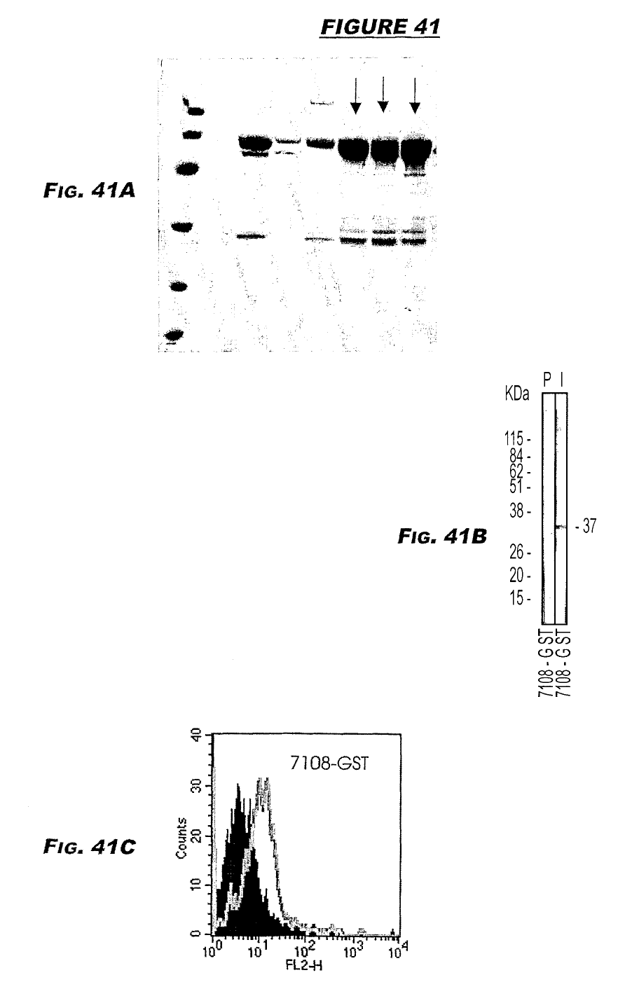

The protein was expressed in E. coli and purified as a GST-fusion product, as shown in FIG. 41A. The recombinant protein was used to immunize mice, whose sera were used in a Western blot (FIG. 41B) and for FACS analysis (FIG. 41C). A his-tagged protein was also expressed.

The cp7108 protein was also identified in the 2D-PAGE experiment.

These experiments show that cp7108 is a surface-exposed and immunoaccessible protein, and that it is a useful immunogen. These properties are not evident from the sequence alone.

### Example 42

The following C. pneumoniae protein (PID 4377287) was expressed <SEQ ID 83; cp7287>:

| | |
|---|---|
| 1 | MVAKKTVRSY RSSFSHSVIV AILSAGIAFE AHSLHSSELD LGVFNKQFEE |
| 51 | HSAHVEEAQT SVLKGSDPVN PSQKESEKVL YTQVPLTQGS SGESLDLADA |
| 101 | NFLEHFQHLF EETTVFGIDQ KLVWSDLDTR NFSQPTQEPD TSNAVSEKIS |
| 151 | SDTKENRKDL ETEDPSKKSG LKEVSSDLPK SPETAVAAIS EDLEISENIS |
| 201 | ARDPLQGLAF FYKNTSSQSI SEKDSSFQGI IFSGSGANSG LGFENLKAPK |
| 251 | SGAAVYSDRD IVFENLVKGL SFISCESLED GSAAGVNIVV THCGDVTLTD |
| 301 | CATGLDLEAL RLVKDFSRGG AVFTARNHEV QNNLAGGILS VVGNKGAIVV |
| 351 | EKNSAEKSNG GAFACGSFVY SNNENTALWK ENQALSGGAI SSASDIDIQG |
| 401 | NCSAIEFSGN QSLIALGEHI GLTDFVGGGA LAAQGTLTLR NNAVVQCVKN |
| 451 | TSKTHGGAIL AGTVDLNETI SEVAFKQNTA ALTGGALSAN DKVIIANNFG |
| 501 | EILFEQNEVR NHGGAIYCGC RSNPKLEQKD SGENINIIGN SGAITFLKNK |
| 551 | ASVLEVMTQA EDYAGGGALW GHNVLLDSNS GNIQFIGNIG GSTFWIGEYV |
| 601 | GGGAILSTDR VTISNNSGDV VFKGNKGQCL AQKYVAPQET APVESDASST |
| 651 | NKDEKSLNAC SHGDHYPPKT VEEEVPPSLL EEHPVVSSTD IRGGGAILAQ |
| 701 | HIFITDNTGN LRFSGNLGGG EESSTVGDLA IVGGGALLST NEVNVCSNQN |
| 751 | VVFSDNVTSN GCDSGGAILA KKVDISANHS VEFVSNGSGK FGGAVCALNE |
| 801 | SVNITDNGSA VSFSKNRTRL GGAGVAAPQG SVTICGNQGN IAFKENFVFG |
| 851 | SENQRSGGGA IIANSSVNIQ DNAGDILFVS NSTGSYGGAI FVGSLVASEG |
| 901 | SNPRTLTITG NSGDILFAKN STQTAASLSE KDSFGGGAIY TQNLKIVKNA |
| 951 | GNVSFYGNRA PSGAGVQIAD GGTVCLEAFG GDILFEGNIN FDGSFNAIHL |
| 1001 | CGNDSKIVEL SAVQDKNIIF QDAITYEENT IRGLPDKDVS PLSAPSLIFN |
| 1051 | SKPQDDSAQH HEGTIRFSRG VSKIPQIAAI QEGTLALSQN AELWLAGLKQ |
| 1101 | ETGSSIVLSA GSILRIFDSQ VDSSAPLPTE NKEETLVSAG VQINMSSPTP |
| 1151 | NKDKAVDTPV LADIISITVD LSSFVPEQDG TLPLPPEIII PKGTKLHSNA |
| 1201 | IDLKIIDPTN VGYENHALLS SHKDIPLISL KTAEGMTGTP TADASLSNIK |
| 1251 | IDVSLPSITP ATYGHTGVWS ESKMEDGRLV VGWQPTGYKL NPEKQGALVL |
| 1301 | NNLWSHYTDL RALKQEIFAH HTIAQRMELD FSTNVWGSGL GVVEDCQNIG |
| 1351 | EFDGFKHHLT GYALGLDTQL VEDFLIGGCF SQFFGKTESQ SYKAKNDVKS |
| 1401 | YMGAAYAGIL AGPWLIKGAF VYGNINNDLT TDYGTLGIST GSWIGKGFIA |
| 1451 | GTSIDYRYIV NPRRFISAIV STVVPFVEAE YVRIDLPEIS EQGKEVRTFQ |
| 1501 | KTRFENVAIP EGFALEHAYS RGSRAEVNSV QLAYVFDVYR KGPVSLITLK |
| 1551 | DAAYSWKSYG VDIPCKAWKA RLSNNTEWNS YLSTYLAFNY EWREDLIAYD |
| 1601 | FNGGIRIIF* |

A predicted signal peptide is highlighted.

The cp7287 nucleotide sequence <SEQ ID 84> is:

| | |
|---|---|
| 1 | ATGGTAGCGA AAAAAACAGT ACGATCTTAT AGGTCTTCAT TTTCTCATTC |
| 51 | CGTAATAGTA GCAATATTGT CAGCAGGCAT TGCTTTTGAA GCACATTCCT |
| 101 | TACACAGCTC AGAACTAGAT TTAGGTGTAT TCAATAAACA GTTTGAGGAA |
| 151 | CATTCTGCTC ATGTTGAAGA GGCTCAAACA TCTGTTTTAA AGGGATCAGA |
| 201 | TCCTGTAAAT CCCTCTCAGA AAGAATCCGA GAAGGTTTTG TACACTCAAG |
| 251 | TGCCTCTTAC CCAAGGAAGC TCTGGAGAGA GTTTGGATCT CGCCGATGCT |
| 301 | AATTTCTTAG AGCATTTTCA GCATCTTTTT GAAGAGACTA CAGTATTTGG |
| 351 | TATCGATCAA AAGCTGGTTT GGTCAGATTT AGATACTAGG AATTTTTCCC |
| 401 | AACCCACTCA AGAACCTGAT ACAAGTAATG CTGTAAGTGA GAAAATCTCC |
| 451 | TCAGATACCA AAGAGAATAG AAAAGACCTA GAGACTGAAG ATCCTTCAAA |
| 501 | AAAAAGTGGC CTTAAAGAAG TTTCATCAGA TCTCCCTAAA AGTCCTGAAA |
| 551 | CTGCAGTAGC AGCTATTTCT GAAGATCTTG AAATCTCAGA AAACATTTCA |
| 601 | GCAAGAGATC CTCTTCAGGG TTTAGCATTT TTTTATAAAA ATACATCTTC |
| 651 | TCAGTCTATC TCTGAAAAGG ATTCTTCATT TCAAGGAATT ATCTTTTCTG |
| 701 | GTTCAGGAGC TAATTCAGGG CTAGGTTTTG AAAATCTTAA GGCGCCGAAA |
| 751 | TCTGGGGCTG CAGTTTATTC TGATCGAGAT ATTGTTTTTG AAAATCTTGT |
| 801 | TAAAGGATTG AGTTTTATAT CTTGTGAATC TTTAGAAGAT GGCTCTGCCG |
| 851 | CAGGTGTAAA CATTGTTGTG ACCCATTGTG GTGATGTAAC TCTCACTGAT |
| 901 | TGTGCCACTG GTTTAGACCT TGAAGCTTTA CGTCTGGTTA AAGATTTTTC |
| 951 | TCGTGGAGGA GCTGTTTTCA CTGCTCGCAA CCATGAAGTG CAAAATAACC |
| 1001 | TTGCAGGTGG AATTCTATCC GTTGTAGGCA ATAAAGGAGC TATTGTTGTA |
| 1051 | GAGAAAAATA GTGCTGAGAA GTCCAATGGA GGAGCTTTTG CTTGCGGAAG |
| 1101 | TTTTGTTTAC AGTAACAACG AAAACACCGC CTTGTGGAAA GAAAATCAAG |
| 1151 | CATTATCAGG AGGAGCCATA TCCTCAGCAA GTGATATTGA TATTCAAGGG |
| 1201 | AACTGTAGCG CTATTGAATT TTCAGGAAAC CAGTCTCTAA TTGCTCTTGG |
| 1251 | AGAGCATATA GGGCTTACAG ATTTTGTAGG TGGAGGAGCT TTAGCTGCTC |
| 1301 | AAGGGACGCT TACCTTAAGA AATAATGCAG TAGTGCAATG TGTTAAAAAC |
| 1351 | ACTTCTAAAA CACATGGTGG AGCTATTTTA GCAGGTACTG TTGATCTCAA |
| 1401 | CGAAACAATT AGCGAAGTTG CCTTTAAGCA GAATACAGCA GCTCTAACTG |
| 1451 | GAGGTGCTTT AAGTGCAAAT GATAAGGTTA TAATTGCAAA TAACTTTGGA |
| 1501 | GAAATTCTTT TTGAGCAAAA CGAAGTGAGG AATCACGGAG GAGCCATTTA |
| 1551 | TTGTGGATGT CGATCTAATC CTAAGTTAGA ACAAAAGGAT TCTGGAGAGA |
| 1601 | ACATCAATAT TATTGGAAAC TCCGGAGCTA TCACTTTTTT AAAAAATAAG |
| 1651 | GCTTCTGTTT TAGAAGTGAT GACACAAGCT GAAGATTATG CTGGTGGAGG |
| 1701 | CGCTTTATGG GGGCATAATG TTCTTCTAGA TTCCAATAGT GGGAATATTC |
| 1751 | AATTTATAGG AAATATAGGT GGAAGTACCT TCTGGATAGG AGAATATGTC |
| 1801 | GGTGGTGGTG CGATTCTCTC TACTGATAGA GTGACAATTT CTAATAACTC |
| 1851 | TGGAGATGTT GTTTTTAAAG GAAACAAAGG CCAATGTCTT GCTCAAAAAT |
| 1901 | ATGTAGCTCC TCAAGAAACA GCTCCCGTGG AATCAGATGC TTCATCTACA |
| 1951 | AATAAAGACG AGAAGAGCCT TAATGCTTGT AGTCATGGAG ATCATTATCC |
| 2001 | TCCTAAAACT GTAGAAGAGG AAGTGCCACC TTCATTGTTA GAAGAACATC |
| 2051 | CTGTTGTTTC TTCGACAGAT ATTCGTGGTG GTGGGGCCAT TCTAGCTCAA |
| 2101 | CATATCTTTA TTACAGATAA TACAGGAAAT CTGAGATTCT CTGGGAACCT |
| 2151 | TGGTGGTGGT GAAGAGTCTT CTACTGTCGG TGATTTAGCT ATCGTAGGAG |
| 2201 | GAGGTGCTTT GCTTTCTACT AATGAAGTTA ATGTTTGCAG TAACCAAAAT |
| 2251 | GTTGTTTTTT CTGATAACGT GACTTCAAAT GGTTGTGATT CAGGGGGAGC |
| 2301 | TATTTTAGCT AAAAAAGTAG ATATCTCCGC GAACCACTCG GTTGAATTTG |
| 2351 | TCTCTAATGG TTCAGGGAAA TTCGGTGGTG CCGTTTGCGC TTTAAACGAA |
| 2401 | TCAGTAAACA TTACGGACAA TGGCTCGGCA GTATCATTCT CTAAAAATAG |
| 2451 | AACACGTCTT GGCGGTGCTG GAGTTGCAGC TCCTCAAGGC TCTGTAACGA |
| 2501 | TTTGTGGAAA TCAGGGAAAC ATAGCATTTA AAGAGAACTT TGTTTTTGGC |
| 2551 | TCTGAAAATC AAAGATCAGG TGGAGGAGCT ATCATTGCTA ACTCTTCTGT |
| 2601 | AAATATTCAG GATAACGCAG GAGATATCCT ATTTGTAAGT AACTCTACGG |
| 2651 | GATCTTATGG AGGTGCTATT TTTGTAGGAT CTTTGGTTGC TTCTGAAGGC |
| 2701 | AGCAACCCAC GAACGCTTAC AATTACAGGC AACAGTGGGG ATATCCTATT |
| 2751 | TGCTAAAAAT AGCACGCAAA CAGCCGCTTC TTTATCAGAA AAAGATTCCT |
| 2801 | TTGGTGGAGG GGCCATCTAT ACACAAAACC TCAAAATTGT AAAGAATGCA |
| 2851 | GGGAACGTTT CTTTCTATGG CAACAGAGCT CCTAGTGGTG CTGGTGTCCA |
| 2901 | AATTGCAGAC GGAGGAACTG TTTGTTTAGA GGCTTTTGGA GGAGATATCT |
| 2951 | TATTTGAAGG GAATATCAAT TTTGATGGGA GTTTCAATGC GATTCACTTA |
| 3001 | TGCGGGAATG ACTCAAAAAT CGTAGAGCTT TCTGCTGTTC AAGATAAAAA |
| 3051 | TATTATTTTC CAAGATGCAA TTACTTATGA AGAGAACACA ATTCGTGGCT |
| 3101 | TGCCAGATAA AGATGTCAGT CCTTTAAGTG CCCCTTCATT AATTTTTAAC |
| 3151 | TCCAAGCCAC AAGATGACAG CGCTCAACAT CATGAAGGGA CGATACGGTT |
| 3201 | TTCTCGAGGG GTATCTAAAA TTCCTCAGAT TGCTGCTATA CAAGAGGGAA |
| 3251 | CCTTAGCTTT ATCACAAAAC GCAGAGCTTT GGTTGGCAGG ACTTAAACAG |
| 3301 | GAAACAGGAA GTTCTATCGT ATTGTCTGCG GGATCTATTC TCCGTATTTT |
| 3351 | TGATTCCCAG GTTGATAGCA GTGCGCCTCT TCCTACAGAA AATAAAGAGG |
| 3401 | AGACTCTTGT TTCTGCCGGA GTTCAAATTA ACATGAGCTC TCCTACACCC |
| 3451 | AATAAAGATA AAGCTGTAGA TACTCCAGTA CTTGCAGATA TCATAAGTAT |
| 3501 | TACTGTAGAT TTGTCTTCAT TTGTTCCTGA GCAAGACGGA ACTCTTCCTC |
| 3551 | TTCCTCCTGA AATTATCATT CCTAAGGGAA CAAAATTACA TTCTAATGCC |
| 3601 | ATAGATCTTA AGATTATAGA TCCTACCAAT GTGGGATATG AAAATCATGC |
| 3651 | TCTTCTAAGT TCTCATAAAG ATATTCCATT AATTTCTCTT AAGACAGCGG |
| 3701 | AAGGAATGAC AGGGACGCCT ACAGCAGATG CTTCTCTATC TAATATAAAA |
| 3751 | ATAGATGTAT CTTTACCTTC GATCACACCA GCAACGTATG GTCACACAGG |
| 3801 | AGTTTGGTCT GAAAGTAAAA TGGAAGATGG AAGACTTGTA GTCGGTTGGC |
| 3851 | AACCTACGGG ATATAAGTTA AATCCTGAGA AGCAAGGGGC TCTAGTTTTG |
| 3901 | AATAATCTCT GGAGTCATTA TACAGATCTT AGAGCTCTTA AGCAGGAGAT |
| 3951 | CTTTGCTCAT CATACGATAG CTCAAAGAAT GGAGTTAGAT TTCTCGACAA |
| 4001 | ATGTCTGGGG ATCAGGATTA GGTGTTGTTG AAGATTGTCA GAACATCGGA |
| 4051 | GAGTTTGATG GGTTCAAACA TCATCTCACA GGGTATGCCC TAGGCTTGGA |
| 4101 | TACACAACTA GTTGAAGACT TCTTAATTGG AGGATGTTTC TCACAGTTCT |
| 4151 | TTGGTAAAAC TGAAAGCCAA TCCTACAAAG CTAAGAACGA TGTGAAGAGT |
| 4201 | TATATGGGAG CTGCTTATGC GGGGATTTTA GCAGGTCCTT GGTTAATAAA |
| 4251 | AGGAGCTTTT GTTTACGGTA ATATAAACAA CGATTTGACT ACAGATTACG |
| 4301 | GTACTTTAGG TATTTCAACA GGTTCATGGA TAGGAAAAGG GTTTATCGCA |
| 4351 | GGCACAAGCA TTGATTACCG CTATATTGTA AATCCTCGAC GGTTTATATC |
| 4401 | GGCAATCGTA TCCACAGTGG TTCCTTTTGT AGAAGCCGAG TATGTCCGTA |
| 4451 | TAGATCTTCC AGAAATTAGC GAACAGGGTA AAGAGGTTAG AACGTTCCAA |
| 4501 | AAAACTCGTT TTGAGAATGT CGCCATTCCT TTTGGATTTG CTTTAGAACA |
| 4551 | TGCTTATTCG CGTGGCTCAC GTGCTGAAGT GAACAGTGTA CAGCTTGCTT |
| 4601 | ACGTCTTTGA TGTATATCGT AAGGGACCTG TCTCTTTGAT TACACTCAAG |
| 4651 | GATGCTGCTT ATTCTTGGAA GAGTTATGGG GTAGATATTC CTTGTAAAGC |
| 4701 | TTGGAAGGCT CGCTTGAGCA ATAATACGGA ATGGAATTCA TATTTAAGTA |
| 4751 | CGTATTTAGC GTTTAATTAT GAATGGAGAG AAGATCTGAT AGCTTATGAC |
| 4801 | TTCAATGGTG GTATCCGTAT TATTTTCTAG |

The PSORT algorithm predicts an inner membrane location (0.106).

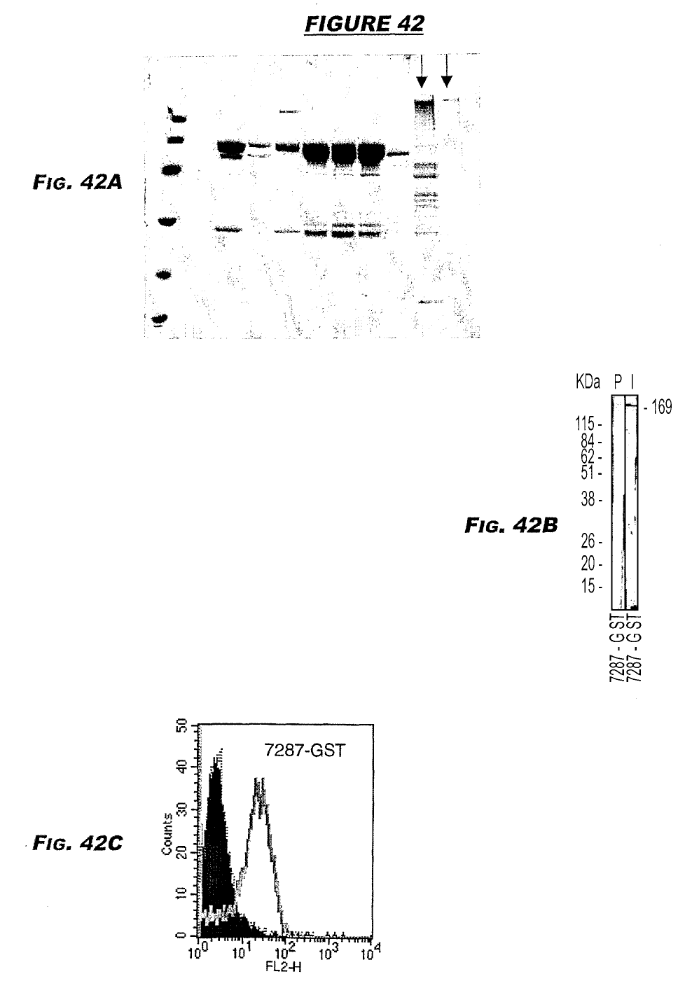

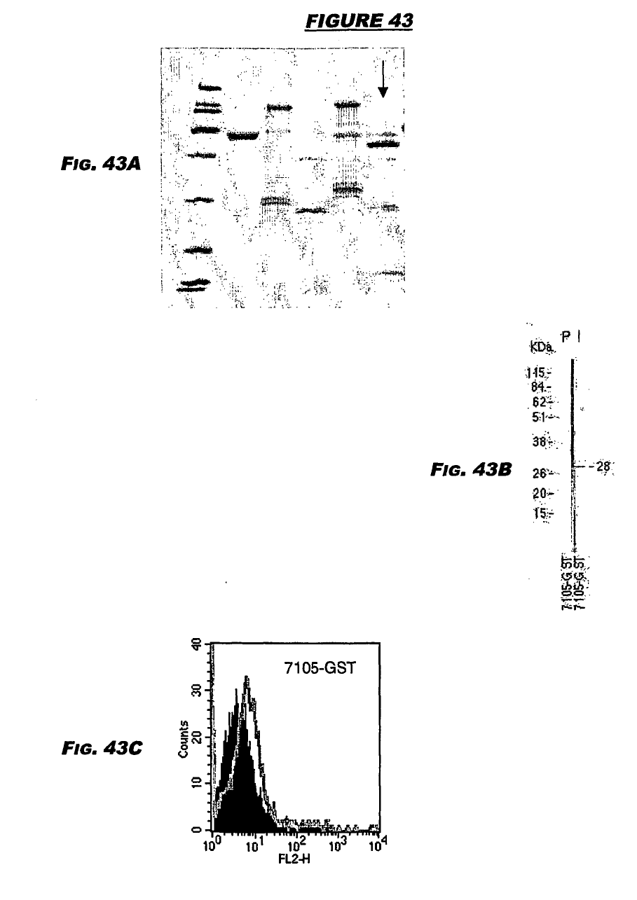

The protein was expressed in E. coli and purified as a GST-fusion product, as shown in FIG. 42A. The recombinant protein was used to immunize mice, whose sera were used in a Western blot (FIG. 42B) and for FACS analysis (FIG. 42C). A his-tagged protein was also expressed.

The cp7287 protein was also identified in the 2D-PAGE experiment and showed good cross-reactivity with human sera, including sera from patients with pneumonitis.

These experiments show that cp7287 is a surface-exposed and immunoaccessible protein, and that it is a useful immunogen. These properties are not evident from the sequence alone.

### Example 43

The following C. pneumoniae protein (PID 4377105) was expressed <SEQ ID 85; cp7105>:

| | |
|---|---|
| 1 | MSLYQKWWNS QLKKSLCYST VAALIFMIPS QESFADSLID LNLGLDPSVE |
| 51 | CLSGDGAFSV GYFTKAGSTP VEYQPFKYDV SKKTFTILSV ETANQSGYAY |
| 101 | GISYDGTITV GTCSLGAGKY NGAKWSADGT LTPLTGITGG TSHTEARAIS |
| 151 | KDTQVIEGFS YDASGQPKAV QWASGATTVT QLADISGGSR SSYAYAISDD |
| 201 | GTIIVGSMES TITRKTTAVK WVNNVPTYLG TLGGDASTGL YISGDGTVIV |
| 251 | GAANTATVTN GNQESHAYMY KDNQMKD* |

The cp7105 nucleotide sequence <SEQ ID 86> is:

| | |
|---|---|
| 1 | GTGAGTCTAT ATCAAAAATG GTGGAACAGT CAGTTAAAGA AGAGCCTCTG |
| 51 | CTATTCGACT GTTGCTGCTC TAATATTTAT GATTCCTTCT CAAGAATCCT |
| 101 | TTGCAGATAG TCTTATAGAT TTAAATTTAG GTTTAGATCC TTCGGTCGAA |
| 151 | TGTCTGTCAG GAGATGGTGC ATTTTCTGTT GGGTATTTTA CTAAGGCGGG |
| 201 | ATCGACTCCC GTAGAATATC AGCCGTTTAA ATACGACGTA TCTAAGAAGA |
| 251 | CATTCACAAT CCTTTCCGTA GAAACGGCAA ATCAGAGCGG CTATGCTTAC |
| 301 | GGAATCTCCT ACGATGGCAC GATCACTGTA GGAACGTGTA GCCTAGGTGC |
| 351 | AGGAAAATAT AACGGCGCAA AATGGAGTGC GGATGGCACT TTAACACCCT |
| 401 | TAACTGGAAT CACGGGGGGG ACGTCACATA CGGAAGCGCG TGCGATTTCT |
| 451 | AAGGATACTC AGGTGATCGA GGGTTTCTCA TATGATGCTT CAGGGCAACC |
| 501 | CAAGGCTGTG CAGTGGGCAA GCGGAGCGAC TACAGTAACA CAATTAGCAG |
| 551 | ATATTTCAGG AGGCTCTAGA AGCTCTTATG CGTATGCTAT ATCTGATGAT |
| 601 | GGCACGATTA TTGTTGGGTC TATGGAGAGC ACGATAACAA GGAAAACTAC |
| 651 | AGCTGTAAAA TGGGTAAATA ATGTTCCTAC GTATCTGGGA ACCTTAGGAG |
| 701 | GAGATGCTTC TACAGGTCTT TATATTTCTG GAGACGGCAC CGTGATTGTA |
| 751 | GGTGCGGCAA ATACAGCAAC TGTAACCAAT GGGAATCAGG AATCCCACGC |
| 801 | CTATATGTAT AAAGATAACC AAATGAAAGA TTGA |

The PSORT algorithm predicts an inner membrane location (0.100).

The protein was expressed in E. coli and purified as a GST-fusion product, as shown in FIG. 43A. The recombinant protein was used to immunize mice, whose sera were used in a Western blot (FIG. 43B) and for FACS analysis (FIG. 43C). A his-tagged protein was also expressed.

This protein also showed good cross-reactivity with human sera, including sera from patients with pneumonitis.

These experiments show that cp7105 is a surface-exposed and immunoaccessible protein, and that it is a useful immunogen. These properties are not evident from the sequence alone.

### Example 44

The following C. pneumoniae protein (PID 4376802) was expressed <SEQ ID 87; cp6802>:

| | |
|---|---|
| 1 | MSNQLQPCIS LGCVSYINSF PLSLQLIKRN DIRCVLAPPA DLLNLLIEGK |
| 51 | LDVALTSSLG AISHNLGYVP GFGIAANQRI LSVNLYAAPT FENSEQERIA |
| 101 | ATLESRSSIG LLKVLCRHLW RIPTPHILRF ITTKVLRQTP ENYDGLLLIG |
| 151 | DAALQHPVLP GFVTYDLASG WYDLTKLPFV FALLLHSTSW KEHELENLAM |
| 201 | EEALQQFESS PEEVLKEAHQ HTGLPPSLLQ EYYALCQYRL GEEHYESFEK |
| 251 | FREYYGTLYQ QARL* |

A predicted signal peptide is highlighted.

The cp6802 nucleotide sequence <SEQ ID 88> is:

| | |
|---|---|
| 1 | ATGTCTAACC AACTCCAGCC ATGTATAAGC TTAGGCTGCG TAAGTTATAT |
| 51 | TAATTCCTTT CCGCTGTCCC TACAACTCAT AAAAAGAAAC GATATTCGCT |
| 101 | GTGTTCTTGC TCCCCCTGCA GACCTCCTCA ACTTGCTAAT CGAAGGGAAA |
| 151 | CTCGATGTTG CTTTGACCTC ATCCCTAGGA GCTATCTCTC ATAACTTGGG |
| 201 | GTATGTCCCC GGCTTTGGAA TTGCAGCAAA CCAACGTATC CTCAGTGTAA |
| 251 | ACCTCTATGC AGCTCCCACT TTCTTTAACT CACCGCAACC TCGGATTGCC |
| 301 | GCAACTTTAG AAAGTCGCTC CTCTATAGGA CTCTTAAAAG TGCTTTGTCG |
| 351 | TCATCTCTGG CGCATCCCAA CTCCTCATAT CCTAAGATTC ATAACTACAA |
| 401 | AAGTACTCAG ACAAACCCCT GAAAATTATG ATGGCCTCCT CCTAATCGGA |
| 451 | GATGCAGCGC TACAACATCC TGTACTTCCT GGATTTGTAA CCTATGACCT |
| 501 | TGCCTCGGGG TGGTATGATC TTACAAAGCT ACCTTTTGTA TTTGCTCTTC |
| 551 | TTCTACACAG CACCTCTTGG AAAGAACATC CCCTACCCAA CCTTGCGATG |
| 601 | GAAGAAGCCC TCCAACAGTT CGAATCTTCA CCCGAAGAAG TCCTTAAAGA |
| 651 | AGCTCATCAA CATACAGGTC TGCCCCCTTC TCTTCTTCAA GAATACTATG |
| 701 | CCCTATGCCA GTACCGTCTA GGAGAAGAAC ACTACGAAAG CTTTGAAAAA |
| 751 | TTCCGGGAAT ATTATGGAAC CCTCTACCAA CAAGCCCGAC TGTAA |

The PSORT algorithm predicts an inner membrane location (0.060).

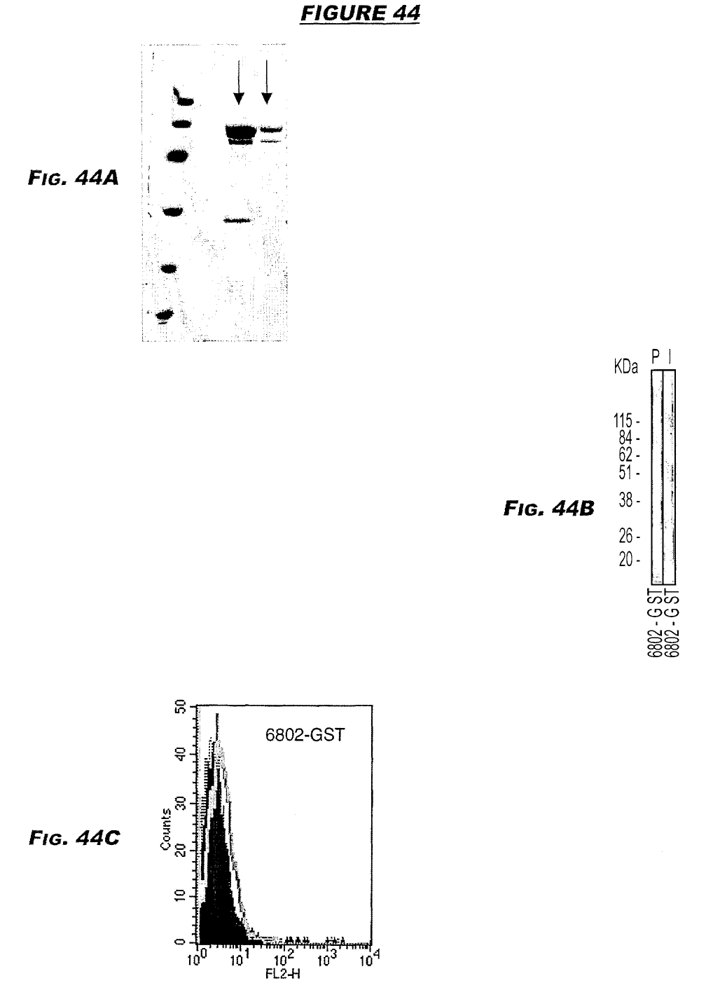

The protein was expressed in E. coli and purified as a GST-fusion product, as shown in FIG. 44A. The recombinant protein was used to immunize mice, whose sera were used in a Western blot (FIG. 44B) and for FACS analysis (FIG. 44C). A his-tagged protein was also expressed.

These experiments show that cp6802 is a surface-exposed and immunoaccessible protein, and that it is a useful immunogen. These properties are not evident from the sequence alone.

### Example 45

The following C. pneumoniae protein (PID 4376390) was expressed <SEQ ID 89; cp6390>:

| | |
|---|---|
| 1 | MVFSYYCMGL FFFSGAISSC GLLVSLGVGL GLSVLGVLLL LLAGLLLFKI |
| 51 | QSMLREVPKA PDLLDLEDAS ERLRVKASRS LASLPKEISQ LESYIRSAAN |
| 101 | DLNTIKTWPH KDQRLVETVS RKLERLAAAQ NYMISELCEI SEILEEEEHH |
| 151 | LILAQESLEW IGKSLFSTFL DMESFLNLSH LSEVRPYLAV NDPRLLEITE |
| 201 | ESWEVVSHFI NVTSAFKKAQ ILFKNNEHSR MKKKLESVQE LLETFIYKSL |
| 251 | KRSYRELGCL SEKMRIIHDN PLFPWVQDQQ KYAHAKNEFG EIARCLEEFE |
| 301 | KTFFWLDEEC AISYMDCWDF LNESIQNKKS RVDRDYISTK KIALKDRART |
| 351 | YAKVLLEENP TTEGKIDLQD AQRAFERQSQ EFYTLEHTET KVRLEALQQC |
| 401 | FSDLREATNV RQVRFTNSEN ANDLKESFEK IDKERVRYQK EQRLYWETID |
| 451 | RNEQELREEI GESLRLQNRR KGYRAGYDAG RLKGLLRQWK KNLRDVEAHL |
| 501 | EDATMDFEHE VSKSELCSVR ARLEVLEEEL MDMSPKVADI EELLSYEERC |
| 551 | ILPIRENLER AYLQYNKCSE ILSKAKFFFP EDEQLLVSEA NLREVGAQLK |
| 601 | QVQGKCQERA QKFAIFEKHI QEQKSLIKEQ VRSFDLAGVG FLKSELLSIA |
| 651 | CNLYIKAVVK ESIPVDVPCM QLYYSYYEDN EAVVRNRLLN MTERYQNFKR |
| 701 | SLNSIQFNGD VLLRDPVYQP EGHETRLKER ELQETTLSCK KLKVAQDRLS |
| 751 | ELESRLSRR |

A predicted signal peptide is highlighted.

The cp6390 nucleotide sequence <SEQ ID 90> is:

| | |
|---|---|
| 1 | TTGGTATTCT CATACTATTG CATGGGATTA TTTTTTTTCT CTGGAGCTAT |
| 51 | TTCTAGTTGT GGTCTTTTAG TGTCTCTAGG AGTTGGTTTA GGACTTAGTG |
| 101 | TTTTAGGAGT ACTTTTACTT CTCTTAGCAG GTCTTTTGCT TTTTAAGATC |
| 151 | CAAAGTATGC TTCGAGAGGT GCCTAAGGCT CCTGATCTAT TAGATTTAGA |
| 201 | AGATGCAAGT GAACGGCTTA GAGTAAAGGC TAGCCGTTCT TTAGCAAGCC |
| 251 | TCCCGAAGGA AATCAGTCAG CTAGAGAGCT ACATTCGTTC TGCAGCTAAT |
| 301 | GATCTAAATA CAATTAAGAC TTGGCCGCAT AAAGATCAAA GACTCGTCGA |
| 351 | GACCGTGTCA CGAAAATTAG AGCGTCTGGC AGCTGCTCAA AACTATATGA |
| 401 | TTTCTGAACT CTGCGAGATT AGTGAGATTC TTGAGGAAGA GGAGCATCAT |
| 451 | CTAATTTTGG CTCAGGAATC TCTAGAATGG ATAGGTAAGA GTCTATTTTC |
| 501 | TACCTTTCTG GACATGGAAT CTTTTTTAAA TTTGAGCCAT CTATCTGAAG |
| 551 | TGCGTCCGTA CTTAGCTGTA AATGATCCTA GATTATTAGA AATTACCGAA |
| 601 | GAATCTTGGG AAGTAGTGAG TCATTTCATA AATGTAACGT CTGCTTTTAA |
| 651 | GAAAGCTCAG ATTCTTTTTA AGAACAACGA ACATTCTCGG ATGAAGAAGA |
| 701 | AGTTAGAAAG TGTTCAAGAG TTACTGGAAA CATTTATTTA TAAGAGTTTA |
| 751 | AAGAGAAGTT ATCGAGAATT AGGATGCTTA AGTGAAAAGA TGAGAATCAT |
| 801 | TCACGACAAT CCTCTCTTCC CTTGGGTGCA AGATCAGCAG AAGTATGCTC |
| 851 | ATGCTAAGAA TGAATTTGGA GAGATTGCGC GGTGTTTAGA GGAGTTTGAA |
| 901 | AAGACGTTCT TCTGGTTGGA TGAGGAGTGT GCTATTTCTT ACATGGACTG |
| 951 | TTGGGATTTT CTAAATGAGT CTATTCAGAA TAAGAAGTCC AGAGTAGATC |
| 1001 | GAGATTATAT ATCCACGAAG AAAATTGCAT TAAAGGATAG AGCCCGCACT |
| 1051 | TATGCTAAGG TTCTTTTAGA AGAGAATCCG ACTACAGAGG GTAAAATAGA |
| 1101 | TTTGCAAGAC GCTCAAAGAG CCTTTGAGCG TCAAAGTCAG GAGTTTTATA |
| 1151 | CACTAGAGCA TACGGAAACA AAGGTGAGAC TAGAAGCACT TCAACAGTGC |
| 1201 | TTCTCGGATC TTAGGGAGGC GACGAACGTA AGGCAAGTTA GGTTTACAAA |
| 1251 | TTCTGAAAAT GCGAATGATT TAAAGGAGAG TTTCGAGAAG ATAGATAAAG |
| 1301 | AGCGTGTGCG ATATCAAAAA GAGCAAAGGC TCTATTGGGA AACAATAGAT |
| 1351 | CGCAATGAGC AAGAGCTTAG GGAAGAGATT GGGGAGTCGC TTCGTTTACA |
| 1401 | AAATCGGAGA AAAGGGTATA GGGCTGGATA TGATGCTGGG CGTTTAAAAG |
| 1451 | GTTTGTTGCG TCAGTGGAAG AAAAATCTCC GCGATGTGGA AGCCCACCTT |
| 1501 | GAAGATGCAA CTATGGATTT TGAGCATGAA GTAAGCAAGA GCGAATTGTG |
| 1551 | CAGTGTTCGG GCGAGGCTCG AGGTTCTAGA AGAAGAGCTG ATGGATATGT |
| 1601 | CTCCTAAAGT TGCGGATATA GAAGAGTTGT TGTCCTATGA AGAGCGTTGT |
| 1651 | ATTCTTCCTA TTAGGGAAAA TTTAGAAAGG GCATACCTCC AATATAATAA |
| 1701 | GTGTTCTGAA ATTTTATCCA AGGCAAAGTT CTTCTTTCCG GAAGACGAGC |
| 1751 | AATTGCTAGT TTCGGAAGCG AATCTAAGAG AGGTGGGTGC CCAGTTAAAA |
| 1801 | CAAGTACAGG GAAAATGTCA AGAGAGGGCC CAAAAGTTCG CAATATTTGA |
| 1851 | AAAGCATATT CAGGAGCAGA AAAGCCTTAT TAAAGAGCAA GTGCGGAGTT |
| 1901 | TTGATCTAGC GGGAGTTGGG TTTTTAAAGA GTGAGCTTCT TAGTATTGCT |
| 1951 | TGTAACCTTT ATATAAAGGC GGTTGTTAAG GAGTCTATAC CAGTTGATGT |
| 2001 | GCCTTGTATG CAGTTATATT ATAGTTATTA CGAAGATAAT GAAGCTGTAG |
| 2051 | TGCGAAACCG CCTTTTAAAT ATGACGGAGA GGTATCAAAA TTTTAAAAGG |
| 2101 | AGTTTGAATT CCATACAATT TAATGGTGAC GTTCTTTTAC GGGATCCGGT |
| 2151 | CTATCAACCT GAAGGTCATG AGACCAGGCT AAAGGAACGG GAGCTACAAG |
| 2201 | AAACAACTTT GTCTTGTAAG AAATTAAAAG TGGCTCAAGA TCGTCTTTCT |
| 2251 | GAATTAGAGT CAAGGCTGTC TAGGAGATAG |

The PSORT algorithm predicts a periplasmic location (0.932).

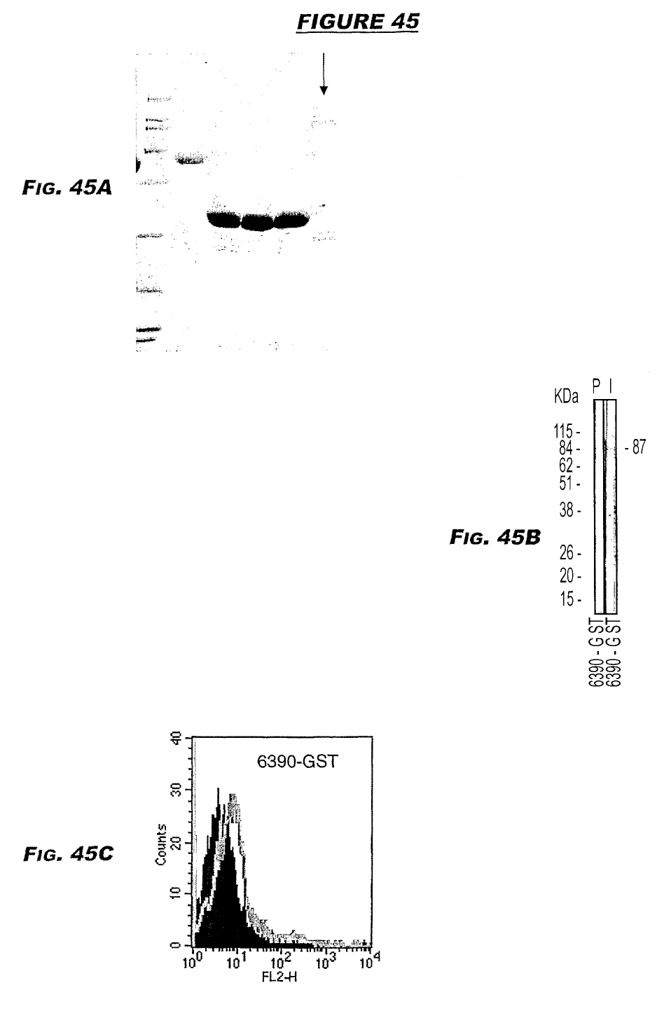

The protein was expressed in E. coli and purified as a GST-fusion product, as shown in FIG. 45A. The recombinant protein was used to immunize mice, whose sera were used in a Western blot (FIG. 45B) and for FACS analysis (FIG. 45C). A his-tagged protein was also expressed.

This protein also showed good cross-reactivity with human sera, including sera from patients with pneumonitis.

These experiments show that cp6390 is a surface-exposed and immunoaccessible protein, and that it is a useful immunogen. These properties are not evident from the sequence alone.

### Example 46

The following C. pneumoniae protein (PID 4376272) was expressed <SEQ ID 91; cp6272>:

| | |
|---|---|
| 1 | MKRCFLFLAS FVLMGSSADA LTHQEAVKKK NSYLSHFKSV SGIVTIEDGV |
| 51 | LNIHNNLRIQ ANKVYVENTV GQSLKLVAHG NVMVNYRAKT LVCDYLEYYE |
| 101 | DTDSCLLTNG RFAMYPWFLG GSMITLTPET IVIRKGYIST SEGPKKDLCL |
| 151 | SGDYLEYSSD SLLSIGKTTL RVCRIPILFL PPFSIMEMEI PKPPINFRGG |
| 201 | TGGFLGSYLG MSYSPISRKH FSSTFFLDSF FKHGVGMGFN LHCSQKQVPE |
| 251 | NVFNMKSYYA HRLAIDMAEA HDRYRLHGDF CFTHKHVNFS GEYHLSDSWE |
| 301 | TVADIFPNNF MLKNTGPTRV DCTWNDNYFE GYLTSSVKVN SFQNANQELP |
| 351 | YLTLRQYPIS IYNTGVYLEN IVECGYLNFA FSDHIVGENF SSLRLAARPK |
| 401 | LHKTVPLPIG TLSSTLGSSL IYYSDVPEIS SRHSQLSAKL QLDYRFLLHK |
| 451 | SYIQRRHIIE PFVTFITETR PLAKNEDHYI FSTQDAFHSL NLLKAGIDTS |
| 501 | VLSKTNPRFP RIHAKLWTTH ILSNTESKPT FPKTACELSL PFGKKNTVSL |
| 551 | DAEWIWKKHC WDHMNIRWEW IGNDNVAMTL ESLHRSKYSL IKCDRENFIL |
| 601 | DVSRPIDQLL DSPLSDHRNL ILGKLFVRPH PCWNYRLSLR YGWHRQDTPN |
| 651 | YLEYQMILGT KIFEHWQLYG VYERREADSR FFFFLKLDKP KKPPF* |

A predicted signal peptide is highlighted.

The cp6272 nucleotide sequence <SEQ ID 92> is:

| | |
|---|---|
| 1 | ATGAAACGTT GCTTCTTATT TCTAGCTTCC TTTGTTCTTA TGGGTTCCTC |
| 51 | AGCTGATGCT TTGACTCATC AAGAGGCTGT GAAAAAGAAA AACTCCTATC |
| 101 | TTAGTCACTT TAAGAGTGTT TCTGGGATTG TGACCATCGA AGATGGGGTA |
| 151 | TTGAATATCC ATAACAACCT GCGGATACAA GCCAATAAAG TGTATGTAGA |
| 201 | AAATACTGTG GGTCAAAGCC TGAAGCTTGT CGCACATGGC AATGTTATGG |
| 251 | TGAACTATAG GGCAAAAACC CTAGTTTGTG ATTACCTAGA GTATTACGAA |
| 301 | GATACAGACT CTTGTCTTCT TACTAATGGA AGATTCGCGA TGTATCCTTG |
| 351 | GTTTCTAGGG GGGTCTATGA TCACTCTAAC CCCAGAAACC ATAGTCATTC |
| 401 | GGAAGGGATA TATCTCTACC TCCGAGGGTC CCAAAAAAGA CCTGTGCCTC |
| 451 | TCCGGAGATT ACCTGGAATA TTCTTCAGAT AGTCTTCTTT CTATAGGGAA |
| 501 | GACAACATTA AGGGTGTGTC GCATTCCGAT ACTTTTCTTA CCTCCATTTT |
| 551 | CTATCATGCC TATGGAGATC CCTAAGCCTC CGATAAACTT TCGAGGAGGA |
| 601 | ACAGGAGGAT TTCTGGGATC CTATTTGGGG ATGAGCTACT CGCCGATTTC |
| 651 | TAGGAAGCAT TTCTCCTCGA CATTTTTCTT GGATAGCTTT TTCAAGCATG |
| 701 | GCGTCGGCAT GGGATTCAAC CTCCATTGTT CTCAGAAGCA GGTTCCTGAG |
| 751 | AATGTCTTCA ATATGAAAAG CTATTATGCC CACCGCCTTG CTATCGATAT |
| 801 | GGCAGAAGCT CATGATCGCT ATCGCCTACA CGGAGATTTC TGCTTCACGC |
| 851 | ATAAGCATGT AAATTTTTCT GGAGAATACC ATCTCAGCGA TAGTTGGGAA |
| 901 | ACTGTTGCTG ACATTTTCCC CAACAACTTC ATGTTGAAAA ATACAGGCCC |
| 951 | CACACGTGTC GATTGCACTT GGAATGACAA CTATTTTGAA GGGTATCTCA |
| 1001 | CCTCTTCTGT TAAGGTAAAC TCTTTCCAAA ATGCCAACCA AGAGCTCCCT |
| 1051 | TATTTAACAT TAAGGCAGTA CCCGATTTCT ATTTATAATA CGGGAGTGTA |
| 1101 | CCTTGAAAAC ATCGTAGAAT GTGGGTATTT AAACTTTGCT TTTAGCGATC |
| 1151 | ATATCGTTGG CGAGAATTTC TCTTCACTAC GTCTTGCTGC GCGCCCTAAG |
| 1201 | CTCCATAAAA CTGTGCCTCT ACCTATAGGA ACGCTCTCCT CCACCCTAGG |
| 1251 | GAGTTCTCTG ATTTACTATA GCGATGTTCC TGAGATCTCC TCGCGCCATA |
| 1301 | GTCAGCTTTC CGCGAAGCTA CAACTTGATT ATCGCTTTCT ATTACATAAG |
| 1351 | TCCTACATTC AAAGACGCCA TATTATAGAG CCGTTCGTTA CCTTCATTAC |
| 1401 | AGAGACTCGT CCTCTAGCTA AGAATGAAGA TCATTATATC TTTTCTATTC |
| 1451 | AAGATGCCTT TCACTCCTTA AACCTTCTGA AAGCGGGTAT AGATACCTCG |
| 1501 | GTACTGAGTA AGACTAACCC TCGATTCCCG AGAATCCATG CGAAGCTGTG |
| 1551 | GACTACCCAC ATCTTGAGCA ATACAGAAAG CAAACCCACG TTTCCCAAAA |
| 1601 | CTGCATGCGA GCTATCTCTA CCTTTTGGAA AGAAAAATAC AGTCTCCTTA |
| 1651 | GATGCTGAAT GGATTTGGAA AAAGCACTGT TGGGATCACA TGAACATACG |
| 1701 | TTGGGAGTGG ATCGGAAATG ACAATGTGGC TATGACTCTA GAATCCCTGC |
| 1751 | ATAGAAGCAA ATACAGCCTG ATTAAGTGTG ACAGGGAGAA CTTCATTTTA |
| 1801 | GATGTCAGCC GTCCCATTGA CCAGCTTTTA GACTCCCCTC TCTCTGATCA |
| 1851 | TAGGAATCTC ATTTTAGGGA AATTATTTGT ACGACCTCAT CCCTGTTGGA |
| 1901 | ATTACCGCTT ATCCTTACGC TATGGCTGGC ATCGCCAGGA CACTCCGAAC |
| 1951 | TACCTAGAAT ACCAGATGAT TCTAGGGACG AAGATCTTCG AACATTGGCA |
| 2001 | GCTCTATGGG GTGTATGAAC GCCGAGAAGC AGATAGTCGA TTTTTCTTCT |
| 2051 | TCTTAAAGCT CGACAAACCT AAAAAACCTC CCTTCTAA |

The PSORT algorithm predicts an outer membrane location (0.48).

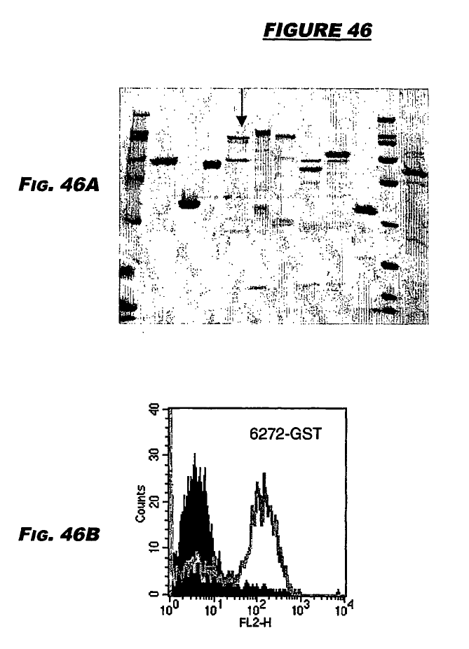

The protein was expressed in E. coli and purified as a GST-fusion product, as shown in FIG. 46A. The recombinant protein was used to immunize mice, whose sera were used in a Western blot and for FACS analysis (FIG. 46B). A his-tagged protein was also expressed.

This protein also showed good cross-reactivity with human sera, including sera from patients with pneumonitis.

These experiments show that cp6272 is a surface-exposed and immunoaccessible protein, and that it is a useful immunogen. These properties are not evident from the sequence alone.

### Example 47

The following C. pneumoniae protein (PID 4377111) was expressed <SEQ ID 93; cp711>:

| | |
|---|---|
| 1 | MFEAVIADIQ AREILDSRGY PTLHVKVTTS TGSVGEARVP SGASTGKKEA |
| 51 | LEFRDTDSPR YQGKGVLQAV KNVKEILFPL VKGCSVYEQS LIDSLMMDSD |
| 101 | GSPNKETLGA NAILGVSLAT AHAAAATLRR PLYRYLGGCF ACSLPCPMMN |
| 151 | LINGGMHADN GLEFQEFMIR PIGASSIKEA VNMGADVFHT LKKLLHERGL |
| 201 | STGVGDEGGF APNLASNEEA LELLLLAIEK AGFTPGKDIS LALDCAASSF |
| 251 | YNVKTGTYDG RHYEEQIAIL SNLCDRYPID SIEDGLAEED YDGWALLTEV |
| 301 | LGEKVQIVGD DLFVTNPELI LEGISNGLAN SVLIKPNQIG TLTETVYAIK |
| 351 | LAQMAGYTTI ISHRSGETTD TTIADLAVAF NAGQIKTGSL SRSERVAKYN |
| 401 | RLMEIEEELG SEAIFTDSNV FSYEDSEE* |

A predicted signal peptide is highlighted.

The cp7111 nucleotide sequence <SEQ ID 94> is:

| | |
|---|---|
| 1 | ATGTTTGAAG CTGTCATTGC CGATATCCAG GCTAGGGAAA TCTTGGATTC |
| 51 | TCGCGGGTAT CCCACTTTAC ATGTTAAAGT AACCACTAGC ACAGGTTCTG |
| 101 | TTGGAGAAGC TCGGGTTCCT TCAGGAGCAT CCACAGGGAA AAAAGAAGCC |
| 151 | TTAGAGTTTC GTGATACAGA TTCTCCTCGT TATCAAGGCA AAGGGGTTTT |
| 201 | GCAAGCTGTA AAAAACGTAA AAGAAATTCT TTTTCCCCTC GTCAAGGGAT |
| 251 | GTAGTGTTTA TGAGCAATCC TTAATTGATT CTCTGATGAT GGATTCTGAC |
| 301 | GGCTCTCCGA ACAAAGAAAC TCTAGGGGCC AATGCTATTT TAGGAGTCTC |
| 351 | TCTAGCTACA GCACATGCAG CAGCAGCAAC ACTACGCAGA CCTCTGTATC |
| 401 | GTTATTTAGG AGGGTGTTTT GCCTGCAGTC TTCCCTGTCC TATGATGAAT |
| 451 | CTGATCAATG GAGGCATGCA TGCCGATAAC GGCTTGGAGT TCCAAGAATT |
| 501 | TATGATCCGT CCTATTGGAG CCTCTTCCAT CAAAGAAGCT GTCAACATGG |
| 551 | GTGCTGACGT TTTTCATACT TTGAAAAAAT TACTCCATGA AAGAGGCTTA |
| 601 | TCTACTGGAG TGGGTGACGA AGGAGGCTTC GCCCCGAATC TTGCTTCTAA |
| 651 | TGAAGAAGCT CTAGAGCTCC TATTGCTGGC TATTGAAAAA GCAGGCTTTA |
| 701 | CTCCAGGAAA AGATATATCG CTAGCCTTAG ACTGCGCAGC ATCCTCATTC |
| 751 | TATAACGTAA AAACAGGCAC GTATGATGGG AGGCACTATG AAGAGCAAAT |
| 801 | CGCAATCCTT TCTAATTTAT GTGATCGCTA TCCTATAGAC TCCATAGAAG |
| 851 | ATGGTCTTGC TGAAGAAGAC TATGACGGGT GGGCCTTGTT AACTGAAGTT |
| 901 | CTTGGAGAAA AAGTACAGAT TGTGGGTGAT GACCTATTTG TTACAAATCC |
| 951 | GGAATTAATA TTAGAGGGTA TTAGCAATGG ATTAGCGAAC TCTGTGTTGA |
| 1001 | TTAAACCAAA TCAGATAGGG ACGCTTACTG AAACAGTGTA TGCTATCAAG |
| 1051 | CTTGCGCAAA TGGCTGGCTA TACTACAATT ATTTCTCATC GCTCAGGAGA |
| 1101 | AACTACGGAC ACTACGATTG CAGATCTTGC TGTTGCCTTC AACGCCGGTC |
| 1151 | AAATCAAAAC AGGCTCTTTA TCACGTTCTG AGCGTGTTGC AAAATACAAT |
| 1201 | AGACTCATGG AAATTGAAGA AGAGCTTGGA TCCGAAGCAA TTTTCACAGA |
| 1251 | TTCTAATGTA TTTTCTTAC GAGGATTCT GAGGAATAG |

The PSORT algorithm predicts an inner membrane location (0.100).

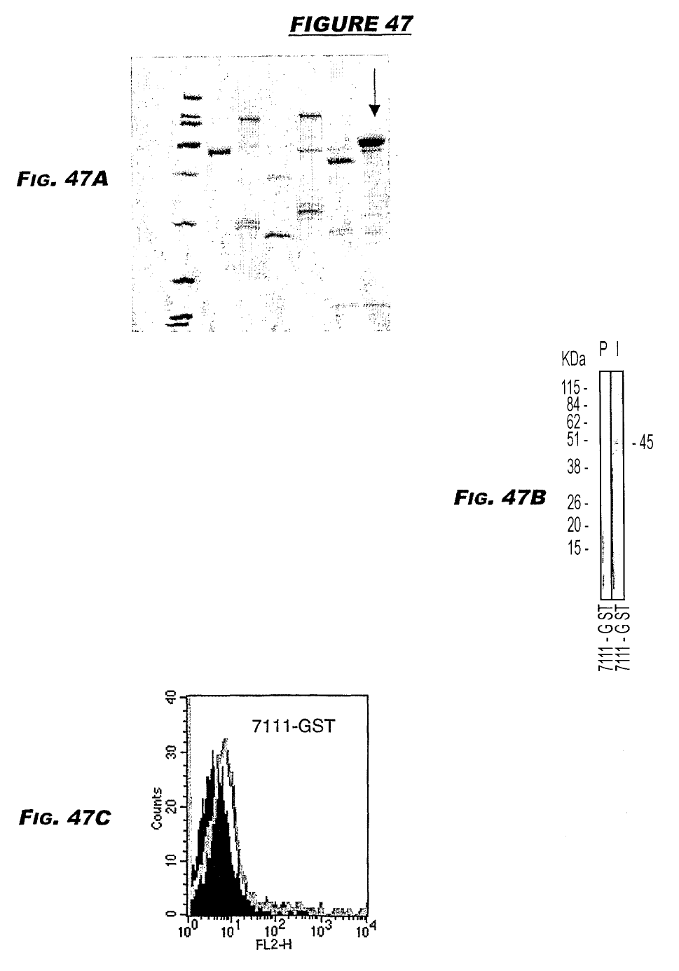

The protein was expressed in E. coli and purified as a GST-fusion product, as shown in FIG. 47A. The recombinant protein was used to immunize mice, whose sera were used in a Western blot (FIG. 47B) and for FACS analysis (FIG. 47C). A his-tagged protein was also expressed.

The cp7111 protein was also identified in the 2D-PAGE experiment and showed good cross-reactivity with human sera, including sera from patients with pneumonitis.

These experiments show that cp7111 is a surface-exposed and immunoaccessible protein, and that it is a useful immunogen. These properties are not evident from the sequence alone.

### Example 48

The following C. pneumoniae protein (PID 4455886) was expressed <SEQ ID 95; cp0010>:

| | |
|---|---|
| 1 | MKSQFSWLVL SSTLACFTSC STVFAATAEN IGPSDSFDGS TNTGTYTPKN |
| 51 | TTTGIDYTLT GDITLQNLGD SAALTKGCFS DTTESLSFAG KGYSLSFLNI |
| 101 | KSSAEGAALS VTTDKNLSLT GFSSLTFLAA PSSVITTPSG KGAVKCGGDL |
| 151 | TFDNNGTILF KQDYCEENGG AISTKNLSLK NSTGSISFEG NKSSATGKKG |
| 201 | GAICATGTVD ITNNTAPTLF SNNIAEAAGG AINSTGNCTI TGNTSLVFSE |
| 251 | NSVTATAGNG GALSGDADVT ISGNQSVTFS GNQAVANGGA IYAKKLTLAS |
| 301 | GGGGVSPFLT IIVQGTTAGN GGAISILAAG ECSLSAEAGD ITFNGNAIVA |
| 351 | TTPQTTKRNS IDIGSTAKIT NLRAISGHSI FFYDPITANT AADSTDTLNL |
| 401 | NKADAGNSTD YSGSIVFSGE KLSEDEAKVA DNLTSTLKQP VTLTAGNLVL |
| 451 | KRGVTLDTKG FTQTAGSSVI MDAGTTLKAS TEEVTLTGLS IPVDSLGEGK |
| 501 | KVVIAASAAS KNVALSGPIL LLDNQGNAYE NHDLGKTQDF SFVQLSALGT |
| 551 | ATTTDVPAVP TVATPTHYGY QGTWGMTWVD DTASTPKTKT ATLAWTNTGY |
| 601 | LPNPERQGPL VPNSLWGSFS DIQAIQGVIE RSALTLCSDR GFWAAGVANF |
| 651 | LDKDKKGEKR KYRHKSGGYA IGGAAQTCSE NLISFAFCQL FGSDKDFLVA |
| 701 | KNHTDTYAGA FYIQHITECS GFIGCLLDKL PGSWSHKPLV LEGQLAYSHV |
| 751 | SNDLKTKYTA YPEVKGSWGN NAFNMMLGAS SHSYPEYLHC FDTYAPYIKL |
| 801 | NLTYIRQDSF SEKGTEGRSF DDSNLFNLSL PIGVKFEKFS DCNDFSYDLT |
| 851 | LSYVPDLIRN DPKCTTALVI SGASWETYAN NLARQALQVR AGSHYAFSPM |
| 901 | FEVLGQFVFE VRGSSRIYNV DLGGKFQF* |

A predicted signal peptide is highlighted.

The cp0010 nucleotide sequence <SEQ ID 96> is:

| | |
|---|---|
| 1 | ATGAAATCGC AATTTTCCTG GTTAGTGCTC TCTTCGACAT TGGCATGTTT |
| 51 | TACTAGTTGT TCCACTGTTT TTGCTGCAAC TGCTGAAAAT ATAGGCCCCT |
| 101 | CTGATAGCTT TGACGGAAGT ACTAACACAG GCACCTATAC TCCTAAAAAT |
| 151 | ACGACTACTG GAATAGACTA TACTCTGACA GGAGATATAA CTCTGCAAAA |
| 201 | CCTTGGGGAT TCGGCAGCTT TAACGAAGGG TTGTTTTTCT GACACTACGG |
| 251 | AATCTTTAAG CTTTGCCGGT AAGGGGTACT CACTTTCTTT TTTAAATATT |
| 301 | AAGTCTAGTG CTGAAGGCGC AGCACTTTCT GTTACAACTG ATAAAAATCT |
| 351 | GTCGCTAACA GGATTTTCGA GTCTTACTTT CTTAGCGGCC CCATCATCGG |
| 401 | TAATCACAAC CCCCTCAGGA AAAGGTGCAG TTAAATGTGG AGGGGATCTT |
| 451 | ACATTTGATA ACAATGGAAC TATTTTATTT AAACAAGATT ACTGTGAGGA |
| 501 | AAATGGCGGA GCCATTTCTA CCAAGAATCT TTCTTTGAAA AACAGCACGG |
| 551 | GATCGATTTC TTTTGAAGGG AATAAATCGA GCGCAACAGG GAAAAAAGGT |
| 601 | GGGGCTATTT GTGCTACTGG TACTGTAGAT ATTACAAATA ATACGGCTCC |
| 651 | TACCCTCTTC TCGAACAATA TTGCTGAAGC TGCAGGTGGA GCTATAAATA |
| 701 | GCACAGGAAA CTGTACAATT ACAGGGAATA CGTCTCTTGT ATTTTCTGAA |
| 751 | AATAGTGTGA CAGCGACCGC AGGAAATGGA GGAGCTCTTT CTGGAGATGC |
| 801 | CGATGTTACC ATATCTGGGA ATCAGAGTGT AACTTTCTCA GGAAACCAAG |
| 851 | CTGTAGCTAA TGGCGGAGCC ATTTATGCTA AGAAGCTTAC ACTGGCTTCC |
| 901 | GGGGGGGGGG GGGTATCTCC TTTTCTAACA ATAaTAGTCC AAGGTACCAC |
| 951 | TGCAGGTAAT GGTGGAGCCA TTTCTATACT GGCAGCTGGA GAGTGTAGTC |
| 1001 | TTTCAGCAGA AGCAGGGGAC ATTACCTTCA ATGGGAATGC CATTGTTGCA |
| 1051 | ACTACACCAC AAACTACAAA AAGAAATTCT ATTGACATAG GATCTACTGC |
| 1101 | AAAGATCACG AATTTACGTG CAATATCTGG GCATAGCATC TTTTTCTACG |
| 1151 | ATCCGATTAC TGCTAATACG GCTGCGGATT CTACAGATAC TTTAAATCTC |
| 1201 | AATAAGGCTG ATGCAGGTAA TAGTACAGAT TATAGTGGGT CGATTGTTTT |
| 1251 | TTCTGGTGAA AAGCTCTCTG AAGATGAAGC AAAAGTTGCA GACAACCTCA |
| 1301 | CTTCTACGCT GAAGCAGCCT GTAACTCTAA CTGCAGGAAA TTTAGTACTT |
| 1351 | AAACGTGGTG TCACTCTCGA TACGAAAGGC TTTACTCAGA CCGCGGGTTC |
| 1401 | CTCTGTTATT ATGGATGCGG GCACAACGTT AAAAGCAAGT ACAGAGGAGG |
| 1451 | TCACTTTAAC AGGTCTTTCC ATTCCTGTAG ACTCTTTAGG CGAGGGTAAG |
| 1501 | AAAGTTGTAA TTGCTGCTTC TGCAGCAAGT AAAAATGTAG CCCTTAGTGG |
| 1551 | TCCGATTCTT CTTTTGGATA ACCAAGGGAA TGCTTATGAA AATCACGACT |
| 1601 | TAGGAAAAAC TCAAGACTTT TCATTTGTGC AGCTCTCTGC TCTGGGTACT |
| 1651 | GCAACAACTA CAGATGTTCC AGCGGTTCCT ACAGTAGCAA CTCCTACGCA |
| 1701 | CTATGGGTAT CAAGGTACTT GGGGAATGAC TTGGGTTGAT GATACCGCAA |
| 1751 | GCACTCCAAA GACTAAGACA GCGACATTAG CTTGGACCAA TACAGGCTAC |
| 1801 | CTTCCGAATC CTGAGCGTCA AGGACCTTTA GTTCCTAATA GCCTTTGGGG |
| 1851 | ATCTTTTTCA GACATCCAAG CGATTCAAGG TGTCATAGAG AGAAGTGCTT |
| 1901 | TGACTCTTTG TTCAGATCGA GGCTTCTGGG CTGCGGGAGT CGCCAATTTC |
| 1951 | TTAGATAAAG ATAAGAAAGG GGAAAAACGC AAATACCGTC ATAAATCTGG |
| 2001 | TGGATATGCT ATCGGAGGTG CAGCGCAAAC TTGTTCTGAA AACTTAATTA |
| 2051 | GCTTTGCCTT TTGCCAACTC TTTGGTAGCG ATAAAGATTT CTTAGTCGCT |
| 2101 | AAAAATCATA CTGATACCTA TGCAGGAGCC TTCTATATCC AACACATTAC |
| 2151 | AGAATGTAGT GGGTTCATAG GTTGTCTCTT AGATAAACTT CCTGGCTCTT |
| 2201 | GGAGTCATAA ACCCCTCGTT TTAGAAGGGC AGCTCGCTTA TAGCCACGTC |
| 2251 | AGTAATGATC TGAAGACAAA GTATACTGCG TATCCTGAGG TGAAAGGTTC |
| 2301 | TTGGGGGAAT AATGCTTTTA ACATGATGTT GGGAGCTTCT TCTCATTCTT |
| 2351 | ATCCTGAATA CCTGCATTGT TTTGATACCT ATGCTCCATA CATCAAACTG |
| 2401 | AATCTGACCT ATATACGTCA GGACAGCTTC TCGGAGAAAG GTACAGAAGG |
| 2451 | AAGATCTTTT GATGACAGCA ACCTCTTCAA TTTATCTTTG CCTATAGGGG |
| 2501 | TGAAGTTTGA GAAGTTCTCT GATTGTAATG ACTTTTCTTA TGATCTGACT |
| 2551 | TTATCCTATG TTCCTGATCT TATCCGCAAT GATCCCAAAT GCACTACAGC |
| 2601 | ACTTGTAATC AGCGGAGCCT CTTGGGAAAC TTATGCCAAT AACTTAGCAC |
| 2651 | GACAGGCCTT GCAAGTGCGT GCAGGCAGTC ACTACGCCTT CTCTCCTATG |
| 2701 | TTTGAAGTGC TCGGCCAGTT TGTCTTTGAA GTTCGTGGAT CCTCACGGAT |
| 2751 | TTATAATGTA GATCTTGGGG GTAAGTTCCA ATTCTAG |

The PSORT algorithm predicts an outer membrane location (0.922).

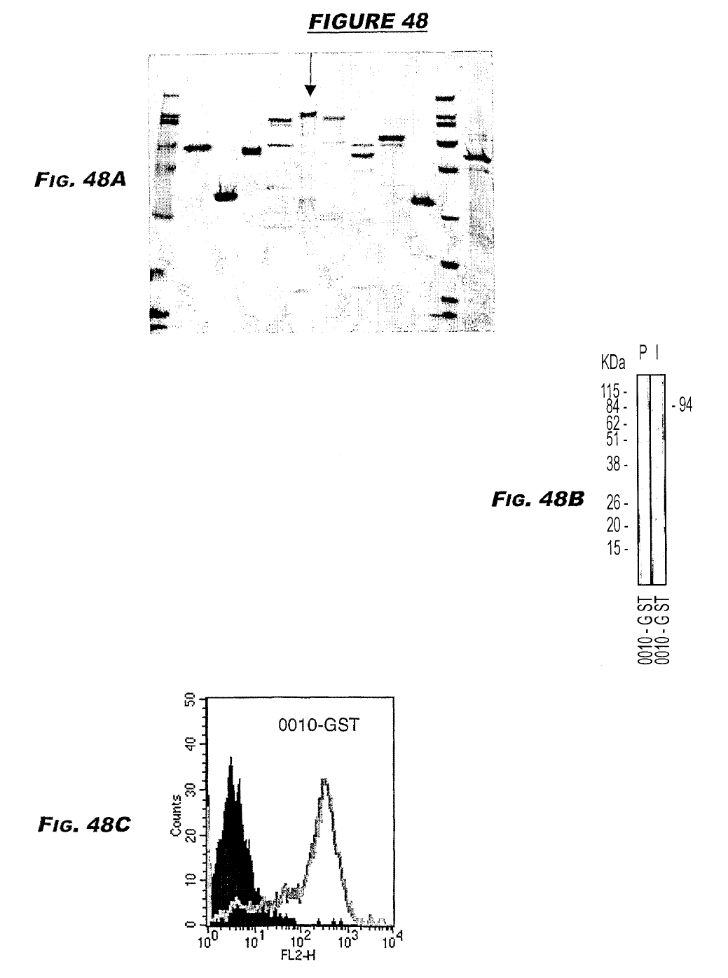

The protein was expressed in E. coli and purified as a GST-fusion product, as shown in FIG. 48A. The recombinant protein was used to immunize mice, whose sera were used in a Western blot (FIG. 48B) and for FACS analysis (FIG. 48C). A his-tagged protein was also expressed.

The cp0010 protein was also identified in the 2D-PAGE experiment and showed good cross-reactivity with human sera, including sera from patients with pneumonitis.

These experiments show that cp0010 is a surface-exposed and immunoaccessible protein, and that it is a useful immunogen. These properties are not evident from the sequence alone.

### Example 49

The following C. pneumoniae protein (PID 4376296) was expressed <SEQ ID 97; cp6296>:

| | |
|---|---|
| 1 | MEEVSEYLQQ VENQLESCSK RLTKMETFAL GVRLEAKEEI ESIILSDVVN |
| 51 | RFEVLCRDIE DMLSRVEEIE RMLRMAELPL LPIKEALTKA FVQHNSCKEK |
| 101 | LTKVEPYFKE SPAYLTSEER LQSLNQTLQR AYKESQKVSG LESEVRACRE |
| 151 | QLKDQVRQFE TQGVSLIKEE ILFVTSTFRT KFSYHSFRLH VPCMRLYEEY |
| 201 | YDDIDLERTR ARWMAMSERY RDAFQAFQEM LKEGLVEEAQ ALRETEYWLY |
| 251 | REERKSKKKH* |

The cp6296 nucleotide sequence <SEQ ID 98> is:

| | |
|---|---|
| 1 | ATGGAGGAGG TGTCTGAGTA TCTTCAGCAA GTAGAAAATC AGTTGGAATC |
| 51 | CTGTTCCAAG CGATTAACCA AGATGGAAAC TTTTGCCTTA GGTGTGAGGT |
| 101 | TGGAAGCTAA AGAAGAGATA GAGTCTATCA TACTTTCTGA TGTAGTGAAC |
| 151 | CGTTTTGAGG TTTTATGTAG AGATATTGAA GATATGCTAT CTCGAGTCGA |
| 201 | GGAGATAGAG CGGATGTTAC GTATGGCGGA GCTTCCTCTA CTTCCTATAA |
| 251 | AAGAAGCGCT TACCAAGGCT TTTGTACAAC ATAACAGCTG TAAAGAGAAG |
| 301 | TTAACCAAGG TAGAGCCTTA CTTTAAAGAG AGCCCTGCAT ATCTAACTAG |
| 351 | TGAAGAGCGA TTGCAGAGTT TGAATCAGAC TTTACAACGT GCGTACAAAG |
| 401 | AGTCCCAAAA GGTTTCAGGT TTAGAATCGG AAGTGAGAGC CTGTCGAGAG |
| 451 | CAGCTTAAAG ATCAAGTAAG ACAGTTTGAA ACTCAAGGAG TGAGCTTGAT |
| 501 | AAAAGAAGAG ATTCTCTTTG TGACTAGTAC CTTTAGAACT AAATTTAGCT |
| 551 | ATCATTCATT TCGATTACAT GTTCCTTGCA TGAGGTTGTA TGAGGAGTAT |
| 601 | TATGATGACA TTGATCTAGA GAGAACTCGA GCTCGATGGA TGGCGATGTC |
| 651 | TGAGAGGTAT AGAGATGCTT TTCAGGCATT CCAGGAGATG TTGAAGGAAG |
| 701 | GCCTAGTTGA AGAAGCTCAG GCTCTTAGAG AAACCGAGTA CTGGTTATAT |
| 751 | CGAGAGGAGA GAAAGAGTAA AAAGAAACAT TGA |

The PSORT algorithm predicts a cytoplasmic location (0.523).

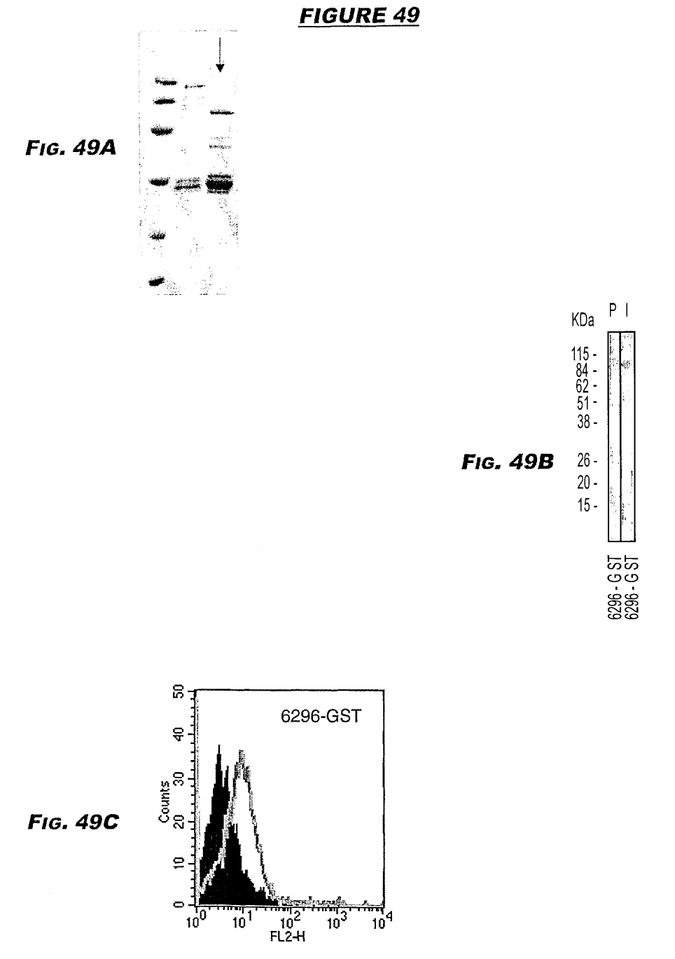

The protein was expressed in E. coli and purified as a GST-fusion product, as shown in FIG. 49A. The recombinant protein was used to immunize mice, whose sera were used in a Western blot (FIG. 49B) and for FACS analysis (FIG. 49C). A his-tagged protein was also expressed.

These experiments show that cp6296 is a surface-exposed and immunoaccessible protein, and that it is a useful immunogen. These properties are not evident from the sequence alone.

### Example 50

The following C. pneumoniae protein (PID 4376664) was expressed <SEQ ID 99; cp6664>:

| | |
|---|---|
| 1 | MVLFHAQASG RNRVKADAIV LPFWHFKDAK NAASFEAEFE PSYLPALENF |
| 51 | QGKTGEIELL YSSPKAKEKR IVLLGLGKNE ELTSDVVFQT YATLTRVLRK |
| 101 | AKCSTVNIIL PTISELRLSA EEFLVGLSSG ILSLNYDYPR YNKVDRNLET |
| 151 | PLSKVTVIGI VPKMADAIFR KEAAIFEGVY LTRDLVNRNA DEITPKKLAE |
| 201 | VALNLGKEFP SIDTKVLGKD AIAKEKMGLL LAVSKGSCVD PHFIVVRYQG |
| 251 | RPKSKDHTVL IGKGVTFDSG GLDLKPKGSM LTMKEDMAGG ATVLGILSAL |
| 301 | AVLELPINVT GIIPATENAI DGASYKMGDV YVGMSGLSVE ICSTDAEGRL |
| 351 | ILADAITYAL KYCKPTRIID FATLTGAMVV SLGEEVAGFF SNNDVLAEDL |
| 401 | LEASAETSEP LWRLPLVKKY DKTLHSDIAD MKNLGSNRAG AITAALFLQR |
| 451 | FLEESSVAWA HLDIAGTAYH EKEEDRYPKY ASGFGVRSIL YYLENSLSK* |

The cp6664 nucleotide sequence <SEQ ID 100> is:

| | |
|---|---|
| 1 | GTGGTTTTAT TTCATGCTCA AGCCTCTGGG CGTAATCGTG TTAAGGCAGA |
| 51 | TGCTATAGTC CTGCCCTTTT GGCATTTTAA GGATGCAAAA AATGCAGCTT |
| 101 | CTTTTGAAGC CGAGTTTGAA CCCTCGTATC TCCCCGCTTT AGAAAACTTT |
| 151 | CAAGGAAAAA CCGGGGAGAT TGAACTCCTT TATAGTAGTC CTAAAGCTAA |
| 201 | GGAAAAACGC ATTGTCCTCT TAGGCTTAGG GAAAAATGAA GAGCTCACCT |
| 251 | CTGATGTTGT TTTCCAAACC TATGCGACAC TAACTCGTGT CTTACGTAAA |
| 301 | GCAAAGTGTT CCACAGTCAA TATCATCTTA CCTACAATTT CTGAATTGCG |
| 351 | GCTTTCTGCC GAAGAATTCT TAGTGGGGTT GTCCTCAGGA ATTTTGTCAT |
| 401 | TAAACTATGA CTACCCACGT TATAATAAGG TAGATCGTAA TCTTGAAACT |
| 451 | CCTCTTTCTA AAGTCACGGT TATCGGTATC GTTCCCAAAA TGGCGGATGC |
| 501 | TATCTTTAGG AAAGAAGCAG CCATTTTCGA AGGCGTATAT CTCACTCGAG |
| 551 | ATCTTGTGAA CAGGAATGCT GATGAAATTA CCCCTAAGAA ATTGGCAGAG |
| 601 | GTTGCTCTGA ATCTGGGAAA AGAGTTCCCT AGTATTGATA CTAAGGTCTT |
| 651 | GGGAAAAGAT GCCATCGCCA AAGAGAAAAT GGGACTCCTA TTGGCTGTTT |
| 701 | CCAAGGGTTC TTGTGTGGAT CCACACTTTA TCGTTGTCCG TTATCAAGGA |
| 751 | CGTCCTAAGT CTAAAGATCA CACCGTCTTG ATAGGGAAAG GGGTCACTTT |
| 801 | TGACTCTGGA GGTTTAGACC TCAAGCCTGG AAAATCCATG CTTACTATGA |
| 851 | AAGAAGACAT GGCAGGTGGG GCTACAGTCC TCGGGATTCT CTCGGCGTTA |
| 901 | GCAGTTTTAG AGCTTCCTAT AAATGTCACG GGGATCATTC CTGCTACAGA |
| 951 | GAATGCTATC GATGGCGCCT CCTATAAAAT GGGAGATGTC TATGTAGGAA |
| 1001 | TGTCGGGGCT TTCTGTTGAG ATTTGTAGTA CCGATGCTGA GGGACGTCTT |
| 1051 | ATCCTCGCTG ATGCGATTAC ATATGCTTTA AAATATTGTA AACCGACACG |
| 1101 | TATTATAGAT TTTGCAACTC TAACAGGAGC TATGGTAGTC TCTCTAGGAG |
| 1151 | AAGAGGTTGC AGGTTTCTTT TCCAATAACG ATGTTTTAGC TGAAGATCTT |
| 1201 | TTAGAGGCGT CAGCCGAAAC CTCCGAGCCG TTATGGAGAC TTCCTCTAGT |
| 1251 | TAAGAAGTAT GATAAAACAT TGCATTCTGA TATTGCTGAT ATGAAAAATC |
| 1301 | TAGGCAGTAA CCGTGCAGGG GCTATTACAG CAGCATTATT CTTGCAGAGA |
| 1351 | TTTTTGGAAG AATCTTCGGT AGCTTGGGCA CATCTTGATA TTGCAGGTAC |
| 1401 | TGCATATCAT GAAAAAGAAG AAGACCGTTA TCCAAAATAT GCTTCAGGTT |
| 1451 | TTGGTGTTCG TTCTATTCTT TATTACTTAG AAAATAGTCT TTCTAAGTAG |

The PSORT algorithm predicts an inner membrane location (0.268).

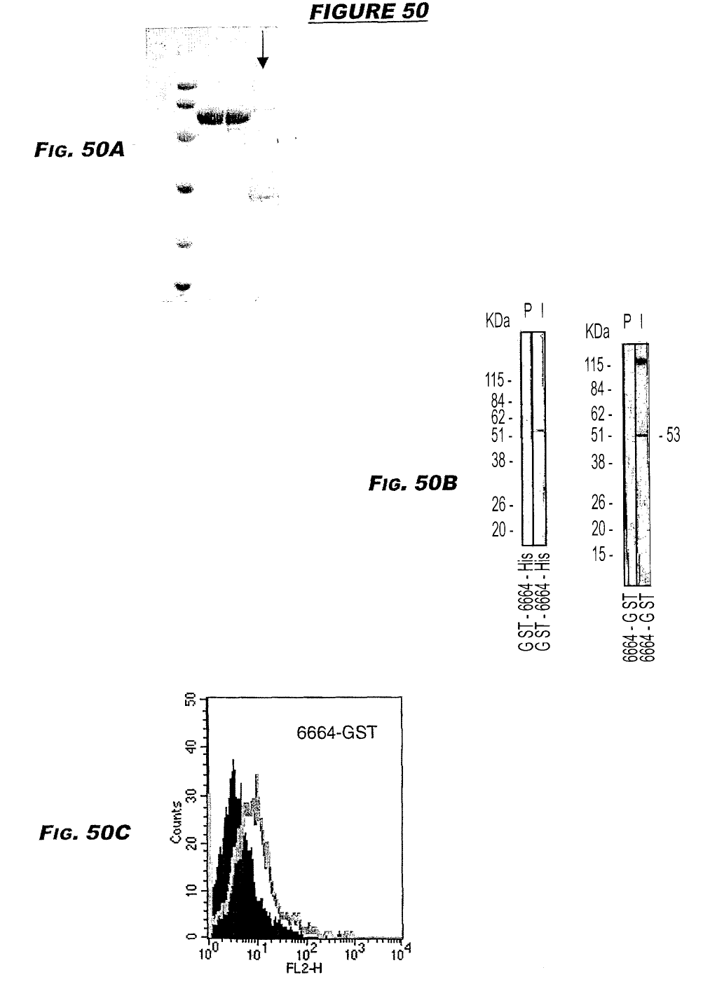

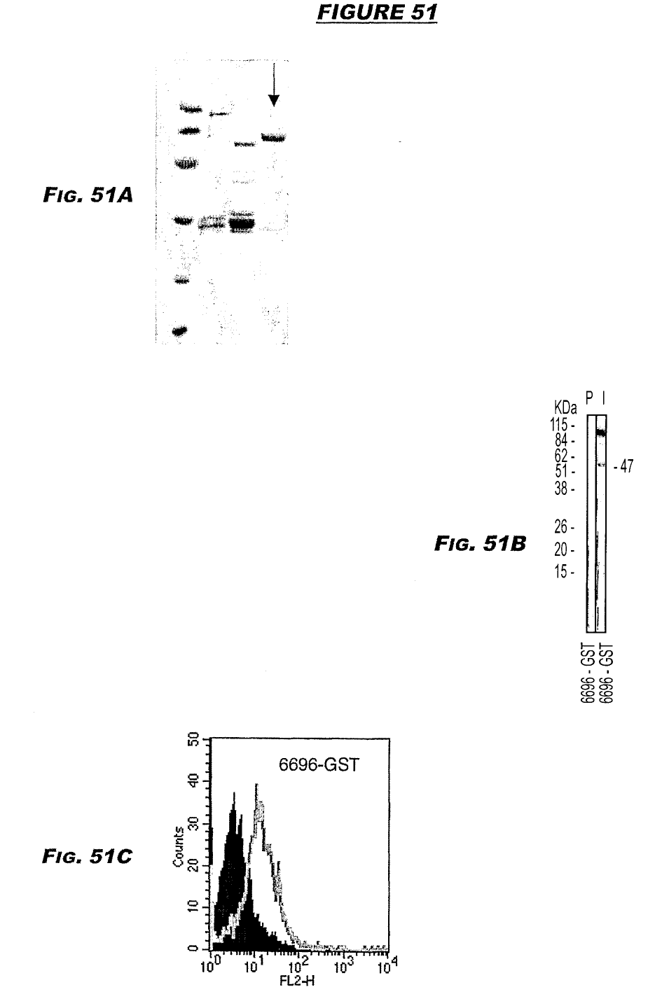

The protein was expressed in E. coli and purified as a GST-fusion (FIG. 50A), as a his-tagged protein, and as a GST/His fusion. The proteins were used to immunize mice, whose sera were used in Western blot Western blot (50B) and FACS (50C) analyses.

The cp6664 protein was also identified in the 2D-PAGE experiment (Cpn0385) and showed good cross-reactivity with human sera, including sera from patients with pneumonitis.

These experiments show that cp6664 is a surface-exposed and immunoaccessible protein, and that it is a useful immunogen. These properties are not evident from the sequence alone.

### Example 51

The following C. pneumoniae protein (PID 4376696) was expressed <SEQ ID 101; cp6696>:

| | |
|---|---|
| 1 | MTLIFVIIIV WCNAFLIKLC VIMGLQSRLQ HCIEVSQNSN FDSQVKQFIY |
| 51 | ACQDKTLRQS VLKIFRYHPL LKIHDIARAV YLLMALEEGE DLGLSFLNVQ |
| 101 | QYPSGAVELF SCGGFPWKGL PYPAEHAEFG LLLLQIAEFY EESQAYVSKM |
| 151 | SHFQQALFDH QGSVFPSLWS QENSRLLKEK TTLSQSFLFQ LQMQIHPEYS |
| 201 | LEDPALGFWM QRTRSSSAFV AASGCQSSLG AYSSGDVGVI AYGPCSGDIS |
| 251 | DCYYFGCCGI AKEFVCQKSH QTTEISFLTS TGKPHPRNTG FSYLRDSYVH |
| 301 | LPIRCKITIS DKQYRVHAAL AEATSAMTFS IFCKGKNCQV VDGPRLRSCS |
| 351 | LDSYKGPGND IMILGENDAI NIVSASPYME IFALQGKEKF WNADFLINIP |
| 401 | YKEEGVMLIF EKKVTSEKGR FFTKMN* |

A predicted signal peptide is highlighted.

The cp6696 nucleotide sequence <SEQ ID 102> is:

| | |
|---|---|
| 1 | TTGACTCTAA TTTTTGTTAT TATTATCGTT TGGTGCAATG CTTTTCTGAT |
| 51 | CAAATTGTGC GTGATAATGG GGCTGCAATC CAGGTTACAA CATTGTATAG |
| 101 | AAGTGTCCCA GAATTCGAAC TTTGATTCAC AAGTAAAACA GTTTATCTAT |
| 151 | GCGTGCCAAG ATAAGACATT AAGGCAGTCT GTACTCAAGA TTTTCCGCTA |
| 201 | CCATCCTTTA CTAAAAATTC ATGATATTGC TCGGGCCGTC TATCTTTTGA |
| 251 | TGGCCTTAGA AGAAGGCGAG GATTTAGGCT TAAGCTTTTT AAATGTACAG |
| 301 | CAGTACCCTT CAGGTGCTGT AGAACTGTTT TCTTGTGGGG GATTTCCTTG |
| 351 | GAAAGGATTA CCTTATCCTG CAGAACATGC GGAATTTGGC CTACTCCTGT |
| 401 | TACAGATCGC AGAGTTTTAT GAAGAGAGTC AGGCATACGT CTCTAAAATG |
| 451 | AGTCATTTTC AACAGGCACT CTTTGATCAC CAAGGGAGCG TCTTTCCCTC |
| 501 | TCTCTGGAGC CAGGAGAACT CTCGACTCCT AAAAGAAAAG ACAACTCTTA |
| 551 | GCCAATCGTT TCTCTTCCAA TTAGGAATGC AAATTCACCC AGAATACAGT |
| 601 | CTTGAGGATC CTGCACTAGG GTTCTGGATG CAAAGAACGC GTTCTTCATC |
| 651 | CGCTTTTGTA GCCGCTTCAG GATGTCAAAG TAGCTTGGGA GCGTATTCCT |
| 701 | CAGGGGATGT CGGTGTTATC GCTTATGGAC CTTGCTCTGG AGACATTAGT |
| 751 | GATTGTTATT ATTTTGGATG TTGTGGAATC GCTAAAGAGT TCGTGTGCCA |
| 801 | AAAATCTCAC CAAACTACAG AGATTTCTTT TCTCACCTCT ACAGGAAAGC |
| 851 | CTCATCCCAG AAATACGGGA TTTTCCTACC TTCGAGATTC CTATGTACAT |
| 901 | CTGCCGATCC GCTGTAAGAT CACTATTTCC GACAAGCAAT ATCGCGTGCA |
| 951 | CGCTGCGTTG GCTGAGGCCA CCTCTGCCAT GACGTTTTCT ATTTTCTGTA |
| 1001 | AGGGGAAGAA TTGTCAGGTT GTTGACGGCC CTCGCTTGCG CTCCTGTTCC |
| 1051 | CTAGATTCTT ATAAAGGTCC CGGAAACGAC ATTATGATTC TTGGGGAAAA |
| 1101 | TGACGCAATC AACATTGTTT CTGCAAGTCC CTATATGGAA ATTTTTGCTT |
| 1151 | TGCAAGGCAA AGAAAAATTT TGGAATGCAG ACTTTTTGAT TAATATTCCT |
| 1201 | TACAAAGAAG AGGGCGTCAT GTTAATTTTT GAAAAAAAAG TGACCTCTGA |
| 1251 | GAAAGGAAGA TTCTTTACGA AGATGAATTA A |

The PSORT algorithm predicts an inner membrane location (0.463).

The protein was expressed in E. coli and purified as a GST-fusion product, as shown in FIG. 51A. The recombinant protein was used to immunize mice, whose sera were used in a Western blot (FIG. 51B) and for FACS analysis (FIG. 51C). A his-tagged protein was also expressed.

This protein also showed good cross-reactivity with human sera, including sera from patients with pneumonitis.

These experiments show that cp6696 is a surface-exposed and immunoaccessible protein, and that it is a useful immunogen. These properties are not evident from the sequence alone.

### Example 52

The following C. pneumoniae protein (PID 4376790) was expressed <SEQ ID 103; cp6790>:

| | |
|---|---|
| 1 | MSEHKKSSKI IGIDLGTTNS CVSVMEGGQA KVITSSEGTR TTPSIVAFKG |
| 51 | NEKLVGIPAK RQAVTNPEKT LGSTKRFIGR KYSEVASEIQ TVPYTVTSGS |
| 101 | KGDAVFEVDG KQYTPEEIGA QILMKMKETA EAYLGETVTE AVITVPAYFN |
| 151 | DSQRASTKDA GRIAGLDVKR IIPEPTAAAL AYGIDKVGDK KIAVFDLGGG |
| 201 | TFDISILEIG DGVFEVLSTN GDTLLGGDDF DEVIIKWMIE EFKKQEGIDL |
| 251 | SKDNMALQRL KDAAEKAKIE LSGVSSTEIN QPFITMDAQG PKHLALTLTR |
| 301 | AQFEKLAASL IERTKSPCIK ALSDAKLSAK DIDDVLLVGG MSRMPAVQET |
| 351 | VKELFGKEPN KGVNPDEVVA IGAAIQGGVL GGEVKDVLLL DVIPLSLGIE |
| 401 | TLGGVMTTLV ERNTTIPTQK KQIFSTAADN QPAVTIVVLQ GERPMAKDNK |
| 451 | EIGRFDLTDI PPAPRGHPQI EVSFDIDANG IFHVSAKDVA SGKEQKIRIE |
| 501 | ASSGLQEDEI QRMVRDAEIN KEEDKKRREA SDAKNEADSM IFRAEKAIKD |
| 551 | YKEQIPETLV KEIEERIENV RNALKDDAPI EKIKEVTEDL SKHMQKIGES |
| 601 | MQSQSASAAA SSAANAKGGP NINTEDLKKH SFSTKPPSNN GSSEDHIEEA |
| 651 | DVEIIDNKKD* |

The cp6790 nucleotide sequence <SEQ ID 104> is:

| | |
|---|---|
| 1 | ATGAGTGAAC ACAAAAAATC AAGCAAAATT ATAGGTATAG ACTTAGGCAC |
| 51 | AACAAACTCC TGCGTATCTG TTATGGAAGG AGGACAAGCT AAAGTAATTA |
| 101 | CATCATCCGA AGGAACAAGA ACCACGCCAT CGATCGTTGC CTTCAAAGGT |
| 151 | AATGAGAAAT TAGTGGGGAT TCCAGCAAAA CGTCAAGCAG TGACAAATCC |
| 201 | AGAAAAAACT CTCGGCTCTA CAAAACGCTT TATTGGCCGT AAGTACTCTG |
| 251 | AAGTAGCTTC GGAAATCCAA ACCGTTCCTT ATACAGTCAC CTCCGGATCT |
| 301 | AAAGGTGATG CCGTTTTCGA AGTTGATGGC AAACAATACA CTCCAGAAGA |
| 351 | AATTGGCGCA CAAATCTTAA TGAAAATGAA AGAGACAGCA GAAGCTTATC |
| 401 | TAGGCGAAAC TGTCACAGAA GCAGTGATCA CCGTCCCCGC ATACTTCAAT |
| 451 | GATTCTCAAC GAGCATCCAC AAAAGATGCT GGACGCATTG CAGGTCTAGA |
| 501 | TGTAAAACGT ATCATTCCAG AACCTACCGC AGCAGCTCTT GCCTACGGAA |
| 551 | TCGATAAAGT CGGTGATAAA AAAATCGCTG TCTTCGACCT TGGTGGAGGA |
| 601 | ACTTTTGATA TCTCCATCCT AGAAATCGGT GATGGCGTCT TCGAAGTTCT |
| 651 | ATCTACAAAT GGAGATACTC TCCTCGGTGG AGACGACTTT GATGAAGTCA |
| 701 | TTATCAAATG GATGATCGAA GAATTCAAAA AACAAGAAGG CATTGATCTT |
| 751 | AGCAAAGATA ATATGGCCTT ACAAAGACTT AAAGATGCTG CTGAGAAAGC |
| 801 | AAAAATAGAA CTTTCAGGAG TCTCTTCCAC AGAAATCAAT CAGCCATTCA |
| 851 | TCACAATGGA TGCACAAGGA CCTAAACACC TTGCATTGAC ACTCACACGT |
| 901 | GCGCAATTCG AGAAACTCGC AGCCTCTCTA ATCGAAAGAA CAAAATCTCC |
| 951 | ATGCATCAAA GCACTCAGTG ACGCAAAACT TTCCGCTAAG GATATCGATG |
| 1001 | ATGTTCTCTT AGTTGGAGGT ATGTCAAGAA TGCCCGCAGT GCAAGAAACT |
| 1051 | GTAAAAGAAC TCTTCGGCAA AGAGCCTAAT AAAGGAGTCA ACCCCGACGA |
| 1101 | AGTTGTTGCT ATTGGAGCCG CAATTCAAGG TGGTGTTCTT GGCGGAGAAG |
| 1151 | TTAAGGATGT TCTACTTCTA GACGTTATCC CCCTATCTCT GGGTATCGAA |
| 1201 | ACTCTAGGAG GCGTCATGAC GACTCTGGTA GAGAGAAATA CTACAATCCC |
| 1251 | TACACAGAAA AAACAAATCT TCTCCACAGC TGCTGATAAC CAGCCTGCGG |
| 1301 | TTACCATCGT AGTTCTCCAA GGAGAGCGTC CCATGGCCAA AGATAACAAG |
| 1351 | GAAATCGGAA GATTCGATCT TACAGATATC CCTCCGGCTC CTCGAGGCCA |
| 1401 | TCCTCAAATC GAAGTCTCCT TCGATATCGA TGCAAACGGA ATTTTCCATG |
| 1451 | TCTCAGCTAA AGATGTTGCC AGCGGTAAAG AACAGAAAAT TCGTATCGAA |
| 1501 | GCAAGCTCAG GACTTCAAGA AGATGAAATC CAAAGAATGG TTCGAGATGC |
| 1551 | CGAAATTAAT AAGGAAGAAG ATAAAAAACG TCGTGAAGCT TCAGATGCTA |
| 1601 | AAAATGAAGC CGATAGCATG ATCTTCAGAG CCGAAAAAGC TATTAAAGAT |
| 1651 | TATAAGGAGC AAATTCCTGA AACTTTAGTT AAAGAAATCG AAGAGCGAAT |
| 1701 | CGAAAACGTG CGCAACGCAC TCAAAGATGA CGCTCCTATT GAAAAAATTA |
| 1751 | AAGAGGTTAC TGAAGACCTA AGCAAGCATA TGCAAAAAAT TGGAGAGTCT |
| 1801 | ATGCAATCGC AGTCTGCATC AGCAGCAGCA TCATCGGCAG CCAATGCTAA |
| 1851 | AGGTGGACCT AACATCAATA CAGAAGATTT GAAAAAACAT AGTTTCAGTA |
| 1901 | CGAAGCCTCC TTCAAATAAC GGTTCTTCAG AAGACCATAT CGAAGAAGCT |
| 1951 | GATGTAGAAA TTATTGATAA CGACGATAAG TAA |

The PSORT algorithm predicts an inner membrane location (0.151).

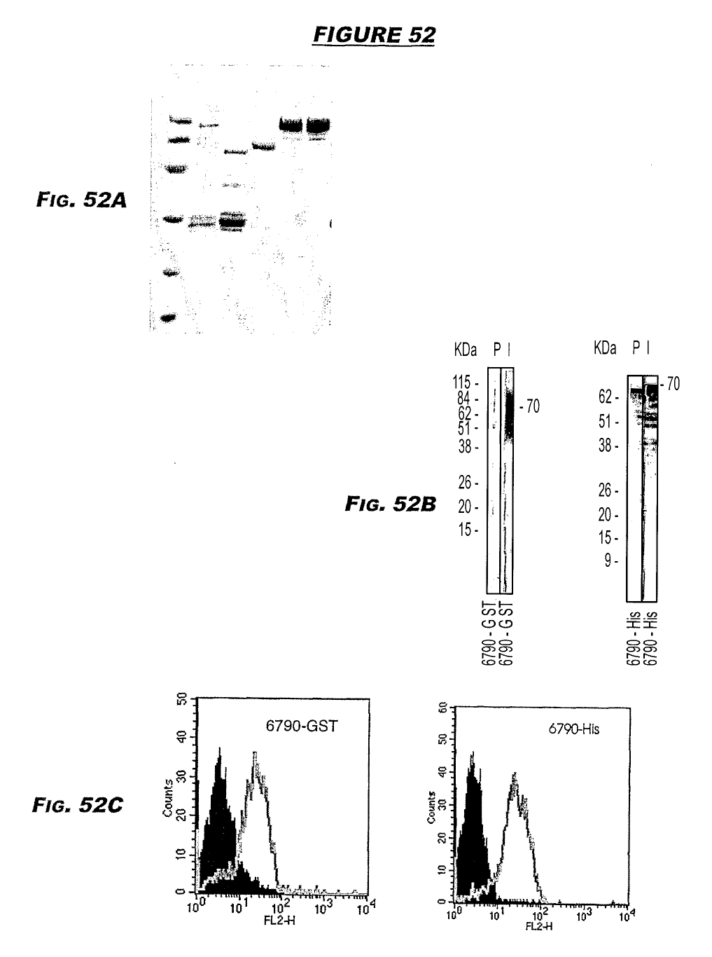

The protein was expressed in E. coli and purified as a GST-fusion product (FIG. 52A) and a his-tagged product. The proteins were used to immunize mice, whose sera were used in Western blot (FIG. 52B) and FACS (FIG. 52C) analyses.

The cp6790 protein was also identified in the 2D-PAGE experiment (Cpn0503).

These experiments show that cp6790 is a surface-exposed and immunoaccessible protein, and that it is a useful immunogen. These properties are not evident from the sequence alone.

### Example 53

The following C. pneumoniae protein (PID 4376878) was expressed <SEQ ID 105; cp6878>:

| | |
|---|---|
| 1 | MNVPDSKNLH PPAYELLEIK ARITQSYKEA SAILTAIPDG ILLLSETGHF |
| 51 | LICNSQAREI LGIDENLEIL NRSFTDVLPD TCLGFSIQEA LESLKVPKTL |
| 101 | RLSLCKESKE KEVELFIRKN EISGYLFIQI RDRSDYKQLE NAIERYKNIA |
| 151 | ELGKMTATLA HEIRNPLSGI VGFASILKKE ISSPRHQRML SSIISGTRSL |
| 201 | NNLVSSMLEY TKSQPLNLKI INLQDFFSSL IPLLSVSFPN CKFVREGAQP |
| 251 | LFRSIDPDRM NSVVWNLVKN AVETGNSPIT LTLHTSGDIS VTNPGTIPSE |
| 301 | IMDKLFTPFF TTKREGNGLG LAEAQKIIRL HGGDIQLKTS DSAVSFFIII |
| 351 | PELLAALPKE RAAS* |

The cp6878 nucleotide sequence <SEQ ID 106> is:

| | |
|---|---|
| 1 | ATGAACGTCC CTGATTCCAA GAACCTCCAT CCTCCTGCAT ACGAACTCCT |
| 51 | AGAGATCAAG GCTCGCATCA CACAATCTTA TAAAGAAGCG AGTGCTATAC |
| 101 | TGACAGCGAT TCCTGATGGT ATCCTATTAC TTTCTGAAAC AGGACACTTT |
| 151 | CTTATCTGCA ATTCACAAGC ACGTGAAATT CTAGGAATTG ATGAAAATCT |
| 201 | AGAAATTCTT AATAGATCCT TTACCGATGT TCTCCCCGAT ACGTGTCTTG |
| 251 | GATTTTCTAT TCAAGAGGCT CTTGAATCTC TAAAAGTCCC TAAAACTCTT |
| 301 | AGACTCTCTC TCTGTAAAGA ATCTAAAGAA AAAGAAGTGG AACTCTTCAT |
| 351 | CCGTAAAAAC GAGATCAGTG GATACCTGTT TATCCAAATC CGCGATCGGT |
| 401 | CCGACTATAA ACAACTAGAA AACGCTATAG AAAGATATAA AAATATCGCA |
| 451 | GAACTTGGGA AAATGACGGC TACCCTAGCT CACGAAATCC GCAATCCGCT |
| 501 | AAGTGGAATC GTTGGATTTG CCTCTATCCT AAAGAAAGAG ATTTCCTCTC |
| 551 | CTCGCCACCA ACGAATGCTC TCCTCAATCA TCTCCGGCAC AAGGTCTCTA |
| 601 | AATAACCTTG TCTCTTCTAT GTTAGAATAT ACAAAATCAC AACCGTTGAA |
| 651 | CCTAAAGATT ATAAATTTAC AAGACTTCTT CTCTTCTCTT ATCCCTCTGC |
| 701 | TCTCCGTCTC TTTCCCGAAT TGCAAGTTTG TAAGAGAGGG CGCACAACCT |
| 751 | CTATTCAGAT CTATAGATCC TGATCGGATG AACAGTGTCG TTTGGAACCT |
| 801 | AGTGAAAAAT GCTGTAGAAA CAGGGAACTC TCCGATCACT CTGACCCTGC |
| 851 | ATACATCGGG AGACATCTCG GTAACGAACC CCGGAACGAT TCCTTCCGAG |
| 901 | ATCATGGACA AGCTCTTCAC TCCATTCTTC ACAACAAAGA GAGAGGGAAA |
| 951 | TGGTTTGGGA CTTGCTGAAG CTCAAAAAAT TATAAGACTC CATGGAGGAG |
| 1001 | ATATCCAATT AAAAACAAGC GACTCCGCCG TTAGCTTCTT CATAATCATC |
| 1051 | CCCGAACTTC TAGCGGCCCT ACCCAAAGAA AGAGCCGCTA G |

The PSORT algorithm predicts an inner membrane location (0.204).

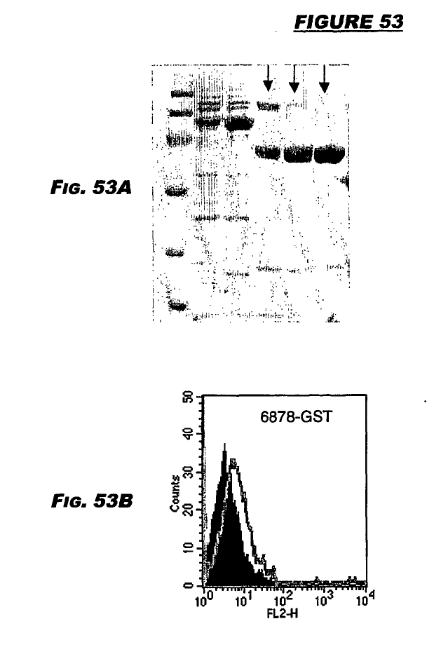

The protein was expressed in E. coli and purified as a his-tag product (FIG. 53A) and as a GST-fusion product. The recombinant GST-fusion protein was used to immunize mice, whose sera were used in a Western blot (FIG. 53B) and for FACS analysis.

These experiments show that cp6878 is a surface-exposed and immunoaccessible protein, and that it is a useful immunogen. These properties are not evident from the sequence alone.

### Example 54

The following C. pneumoniae protein (PID 4377224) was expressed <SEQ ID 107; cp7224>:

| | |
|---|---|
| 1 | MMKKIRKVAL AVGGSGGHIV PALSVKEAFS REGIDVLLLG KGLKNHPSLQ |
| 51 | QGISYREIPS GLPTVLNPIK IMSRTLSLCS GYLKARKELK IFDPDLVIGF |
| 101 | GSYHSLPVLL AGLSHKIPLF LHEQNLVPGK VNQLFSRYAR GIGVNFSPVT |
| 151 | KHFRCPAEEV FLPKRSFSLG SPMMKRCTNH TPTICVVGGS QGAQILNTCV |
| 201 | PQALVKLVNK YPNMYVHHIV GPKSDVMKVQ HVYNRGEVLC CVKPFEEQLL |
| 251 | DVLLAADLVI SRAGATILEE ILWAKVPGIL IPYPGAYGHQ EVNAKFFVDV |
| 301 | LEGGTMILEK ELTEKLLVEK VTFALDSHNR EKQRNSLAAY SQQRSTKTFH |
| 351 | AFICECL* |

The cp7224 nucleotide sequence <SEQ ID 108> is:

| | |
|---|---|
| 1 | ATGATGAAGA AAATTCGAAA AGTAGCCTTG GCTGTAGGAG GTTCAGGAGG |
| 51 | CCACATTGTC CCAGCTCTCT CGGTAAAGGA AGCTTTTTCT CGTGAAGGAA |
| 101 | TAGACGTATT ACTACTAGGG AAAGGTCTCA AGAACCATCC TTCTTTGCAA |
| 151 | CAGGGAATCA GCTATCGGGA AATCCCCTCA GGACTTCCTA CAGTCCTTAA |
| 201 | TCCCATAAAG ATCATGAGCA GGACCCTTTC TCTATGTTCA GGATACCTGA |
| 251 | AAGCAAGAAA GGAACTTAAA ATTTTTGACC CTGACCTGGT CATAGGATTT |
| 301 | GGGAGCTACC ACTCTCTTCC CGTGTTGCTC GCAGGACTGT CCCATAAAAT |
| 351 | TCCCTTATTT CTACACGAAC AAAATCTAGT TCCTGGAAAA GTAAATCAAT |
| 401 | TGTTTTCCCG CTATGCTCGA GGTATTGGAG TGAATTTCTC CCCCGTTACT |
| 451 | AAACACTTCC GCTGCCCCGC AGAAGAGGTC TTCCTTCCTA AACGAAGCTT |
| 501 | CTCCTTAGGA AGCCCTATGA TGAAGCGATG TACAAATCAT ACCCCTACAA |
| 551 | TCTGTGTTGT TGGAGGTTCT CAGGGAGCAC AGATATTAAA TACTTGTGTT |
| 601 | CCCCAAGCTC TTGTCAAGCT AGTCAATAAG TACCCAAATA TGTACGTCCA |
| 651 | TCATATTGTA GGACCTAAAA GTGATGTTAT GAAGGTGCAA CATGTTTACA |
| 701 | ATCGTGGAGA GGTCCTCTGC TGTGTGAAGC CGTTCGAAGA GCAACTCCTA |
| 751 | GATGTCTTGC TTGCCGCAGA TTTGGTCATC AGTAGGGCAG GAGCCACAAT |
| 801 | TTTAGAAGAA ATTCTTTGGG CAAAAGTTCC CGGAATTTTA ATTCCCTATC |
| 851 | CAGGAGCTTA TGGACATCAG GAAGTTAATG CTAAATTCTT TGTAGACGTC |
| 901 | TTAGAAGGGG GAACTATGAT CCTAGAAAAA GAATTAACAG AGAAGCTATT |
| 951 | AGTAGAAAAA GTAACGTTTG CTTTAGACTC CCATAACAGA GAAAAACAAC |
| 1001 | GCAATTCCCT AGCGGCGTAT AGTCAGCAAA GGTCAACAAA AACATTCCAT |
| 1051 | GCATTCATTT GTGAATGCTT ATAG |

The PSORT algorithm predicts an inner membrane location (0.164).

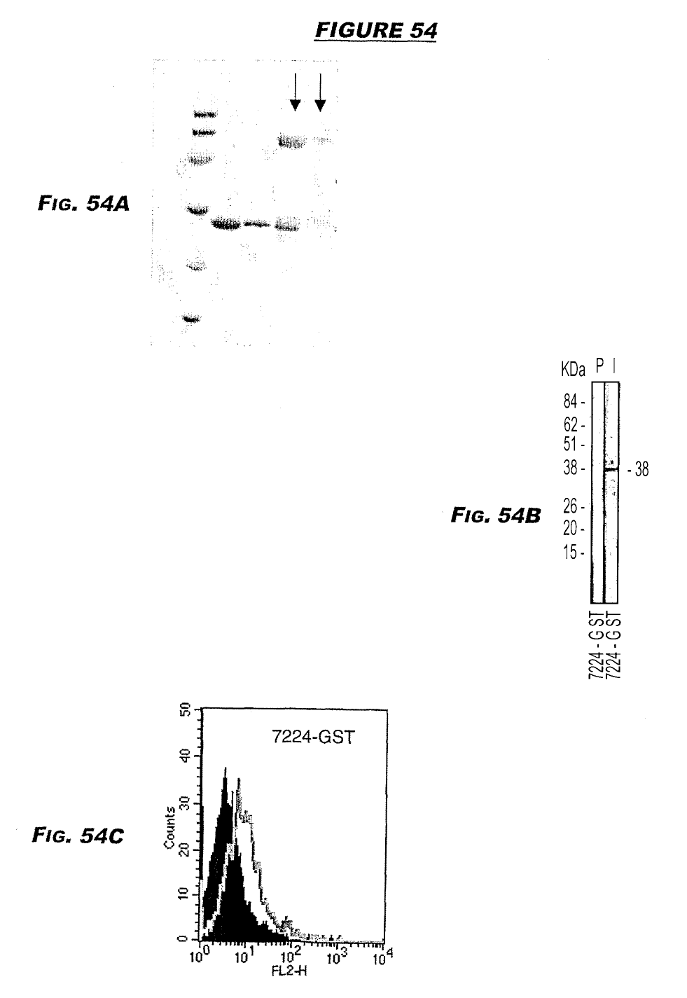

The protein was expressed in E. coli and purified as a GST-fusion product, as shown in FIG. 54A. The recombinant protein was used to immunize mice, whose sera were used in a Western blot (FIG. 54B) and for FACS analysis (FIG. 54C). A his-tagged protein was also expressed.

This protein also showed good cross-reactivity with human sera, including sera from patients with pneumonitis.

These experiments show that cp7224 is a surface-exposed and immunoaccessible protein, and that it is a useful immunogen. These properties are not evident from the sequence alone.

### Example 55

The following C. pneumoniae protein (PID 4377140) was expressed <SEQ ID 109; cp7140>:

| | |
|---|---|
| 1 | MVRRSISFCL FFLMTLLCCT SCNSRSLIVH GLPGREANEI VVLLVSKGVA |
| 51 | AQKLPQAAAA TAGAATEQMW DIAVPSAQIT EALAILNQAG LPRMKGTSLL |
| 101 | DLFAKQGLVP SELQEKIRYQ EGLSEQMAST IRKMDGVVDA SVQISFTTEN |
| 151 | EDNLPLTASV YIKHRGVLDN PNSIMVSKIK RLIASAVPGL VPENVSVVSD |
| 201 | RAAYSDITIN GPWGLTEEID YVSVWGIILA KSSLTKFRLI FYVLILILFV |
| 251 | ISCGLLWVIW KTHTLIMTMG GTKGFFNPTP YTKNALEAKK AEGAAADKEK |
| 301 | KEDADSQGES KNAETSDKDS SDKDAPEGSN EIEGA* |

A predicted signal peptide is highlighted.

The cp7140 nucleotide sequence <SEQ ID 110> is:

| | |
|---|---|
| 1 | ATGGTTCGTC GATCTATTTC TTTTTGCTTG TTCTTTCTAA TGACATTGCT |
| 51 | GTGCTGTACA AGCTGTAACA GCAGGTCTCT AATTGTGCAC GGTCTTCCTG |
| 101 | GCAGAGAAGC GAATGAGATT GTGGTGCTTT TGGTAAGCAA AGGGGTGGCT |
| 151 | GCACAAAAAT TGCCTCAAGC TGCAGCGGCT ACAGCCGGAG CAGCTACTGA |
| 201 | GCAAATGTGG GATATCGCGG TTCCGTCAGC ACAAATCACA GAGGCCCTTG |
| 251 | CCATTCTAAA TCAAGCGGGT CTTCCACGTA TGAAAGGGAC AAGCCTGTTA |
| 301 | GATCTTTTTG CAAAACAAGG TCTTGTTCCT TCCGAGCTTC AGGAAAAAAT |
| 351 | CCGTTATCAA GAAGGCTTAT CAGAACAGAT GGCCTCTACG ATTAGAAAAA |
| 401 | TGGATGGCGT TGTCGATGCC TCAGTACAGA TTTCCTTCAC TACAGAAAAT |
| 451 | GAAGATAATC TTCCTTTAAC AGCCTCTGTG TATATTAAGC ATCGAGGGGT |
| 501 | TTTGGACAAT CCGAACAGCA TTATGGTTTC CAAAATTAAG CGCCTTATTG |
| 551 | CAAGTGCTGT TCCAGGACTT GTGCCAGAGA ACGTCTCTGT AGTGAGCGAT |
| 601 | CGCGCAGCTT ATAGTGATAT TACAATTAAT GGTCCTTGGG GATTAACAGA |
| 651 | AGAAATCGAT TATGTTTCTG TTTGGGGTAT TATTCTTGCG AAGTCTTCGC |
| 701 | TCACCAAATT CCGTCTCATT TTTTATGTCT TGATTCTCAT TTTATTTGTT |
| 751 | ATTTCTTGTG GTCTCCTTTG GGTCATTTGG AAAACTCATA CTCTCATTAT |
| 801 | GACTATGGGA GGTACAAAAG GGTTCTTCAA CCCTACACCA TATACAAAGA |
| 851 | ATGCCTTGGA AGCCAAGAAA GCCGAGGGAG CAGCTGCTGA CAAAGAGAAA |
| 901 | AAAGAAGATG CAGATTCACA GGGGGAAAGC AAAAATGCGG AAACCAGTGA |
| 951 | TAAAGACTCT AGTGATAAAG ATGCTCCAGA AGGAAGCAAT GAAATTGAGG |
| 1001 | GTGCTTAG |

The PSORT algorithm predicts an inner membrane location (0.650).

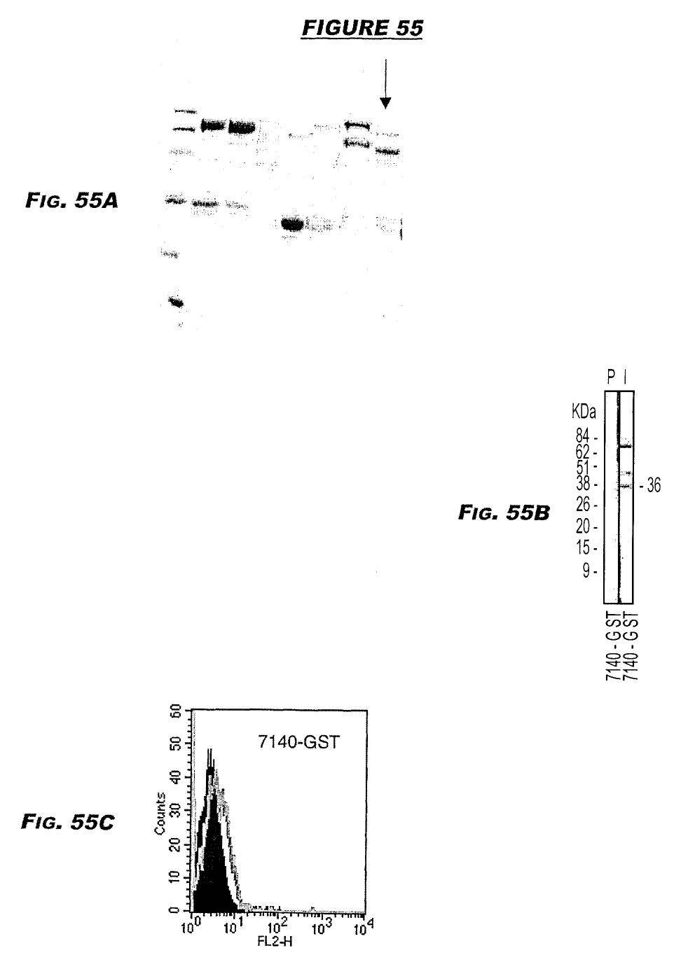

The protein was expressed in E. coli and purified as a GST-fusion product, as shown in FIG. 55A. The recombinant protein was used to immunize mice, whose sera were used in a Western blot (FIG. 55B) and for FACS analysis (FIG. 55C). A his-tagged protein was also expressed.

These experiments show that cp7140 is a surface-exposed and immunoaccessible protein, and that it is a useful immunogen. These properties are not evident from the sequence alone.

### Example 56

The following C. pneumoniae protein (PID 4377306) was expressed <SEQ ID 111; cp7306>:

| | |
|---|---|
| 1 | MITKQLRSWL AVLVGSSLLA LPLSGQAVGK KESRVSELPQ DVLLKEISGG |
| 51 | FSKVATKATP AVVYIESFPK SQAVTHPSPG RRGPYENPFD YFNDEFFNRF |
| 101 | FGLPSQREKP QSKEAVRGTG FLVSPDGYIV TNNHVVEDTG KIHVTLHDGQ |
| 151 | KYPATVIGLD PKTDLAVIKI KSQNLPYLSF GNSDHLKVGD WAIAIGNPFG |
| 201 | LQATVTVGVI SAKGRNQLHI ADFEDFIQTD AAINPGNSGG PLLNIDGQVI |
| 251 | GVNTAIVSGS GGYIGIGFAI PSLMANRIID QLIRDGQVTR GFLGVTLQPI |
| 301 | DAELAACYKL EKVYGALVTD VVKGSPADKA GLKQEDVIIA YNGKEVDSLS |
| 351 | MFRNAVSLMN PDTRIVLKVV REGKVIEIPV TVSQAPKEDG MSALQRVGIR |
| 401 | VQNLTPETAK KLGIAPETKG ILIISVEPGS VAASSGIAPG QLILAVNRQK |
| 451 | VSSIEDLNRT LKDSNNENIL LMVSQGDVIR FIALKPEE* |

A predicted signal peptide is highlighted.

The cp7306 nucleotide sequence <SEQ ID 112> is:

```
      1 ATGATAACTA AGCAATTGCG TTCGTGGCTA GCTGTACTTG TTGGTTCAAG
    51 TCTGCTAGCT CTTCCTTTAT CAGGGCAAGC TGTCGGGAAA AAAGAATCTC
  101 GAGTTTCCGA GCTGCCTCAA GACGTTCTTC TTAAAGAGAT CTCGGGAGGG
  151 TTTTCTAAGG TCGCTACCAA GGCGACTCCC GCTGTTGTGT ACATAGAAAG
  201 TTTCCCAAAG AGCCAGGCTG TAACACATCC TTCTCCTGGA CGCCGTGGGC
  251 CTTATGAAAA TCCTTTTGAT TATTTTAATG ATGAGTTTTT CAATCGTTTT
  301 TTTGGTCTAC CTTCACAGAG GGAAAAACCT CAAAGTAAAG AGGCGGTTCG
  351 AGGAACAGGT TTCCTAGTAT CTCCAGATGG CTATATTGTG ACTAATAACC
  401 ATGTTGTCGA AGATACAGGT AAGATTCACG TAACTCTTCA TGATGGGCAA
  451 AAGTACCCAG CAACTGTAAT CGGACTCGAT CCTAAAACAG ACCTTGCAGT
  501 CATTAAAATT AAATCCCAAA ACCTCCCGTA TCTTTCTTTT GGAAACTCCG
  551 ACCACTTAAA AGTCGGAGAT TGGGCAATTG CAATTGGAAA TCCCTTCGGT
  601 CTTCAAGCTA CGGTCACCGT AGGTGTCATC AGTGCTAAAG GAAGAAATCA
  651 ACTCCACATT GCAGATTTTG AAGATTTTAT TCAGACAGAT GCTGCGATTA
  701 ATCCAGGCAA CTCTGGAGGC CCTCTTCTAA ATATTGATGG ACAGGTCATC
  751 GGTGTTAATA CTGCCATTGT CAGTGGTAGT GGTGGCTATA TTGGAATCGG
  801 GTTTGCGATT CCTAGCCTTA TGGCAAATAG AATCATAGAT CAGCTGATTC
  851 GTGATGGTCA AGTTACCCGA GGATTCTTAG GAGTGACTTT ACAACCTATA
  901 GATGCGGAAC TCGCTGCTTG CTACAAACTC GAAAAGGTTT ATGGCGCTTT
  951 AGTCACAGAT GTTGTTAAAG GATCTCCAGC AGATAAAGCA GGGCTAAAAC
1001 AAGAAGATGT GATCATTGCT TATAATGGGA AAGAAGTCGA TTCACTGAGT
1051 ATGTTCCGTA ATGCTGTTTC TTTAATGAAT CCAGATACAC GTATTGTTCT
1101 AAAGGTAGTT CGTGAAGGAA AGGTTATCGA AATACCCGTG ACAGTTTCTC
1151 AAGCTCCAAA AGAAGATGGA ATGTCGGCTT TACAGCGTGT GGGAATCCGT
1201 GTGCAAAACC TAACTCCTGA AACTGCTAAG AAGCTGGGAA TTGCTCCAGA
1251 GACTAAAGGC ATTTTGATTA TAAGTGTTGA ACCAGGGTCT GTAGCAGCTT
1301 CTTCAGGAAT TGCTCCTGGT CAGCTGATCC TTGCTGTGAA TAGACAAAAA
1351 GTATCTTCGA TTGAAGATCT GAATAGAACG TTAAAAGATT CTAACAATGA
1401 GAATATTCTT CTTATGGTTT CTCAAGGAGA TGTTATTCGC TTCATTGCCC
1451 TGAAACCTGA AGAATAA
```

The PSORT algorithm predicts a periplasmic location (0.923).

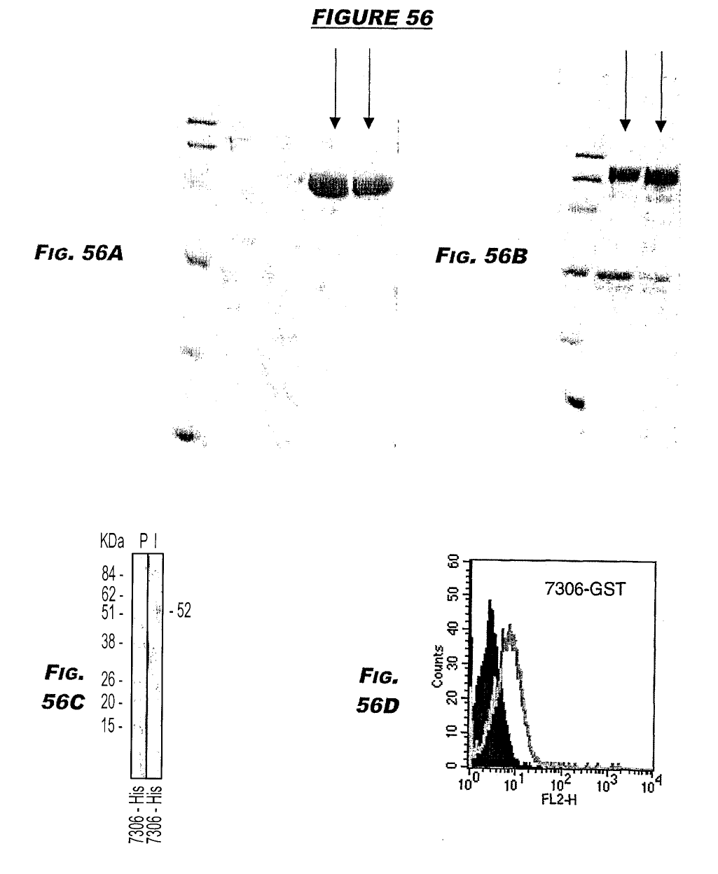

The protein was expressed in E. coli and purified as a his-tag product (FIG. 56A) and as a GST-fusion product (FIG. 56B). The recombinant proteins were used to immunize mice, whose sera were used in a Western blot (FIG. 56C) and for FACS (FIG. 56D) analyses.

The cp7306 protein was also identified in the 2D-PAGE experiment (Cpn0979) and showed good cross-reactivity with human sera, including sera from patients with pneumonitis.

These experiments show that cp7306 is a surface-exposed and immunoaccessible protein, and that it is a useful immunogen. These properties are not evident from the sequence alone.

### Example 57

The following C. pneumoniae protein (PID 4377132) was expressed <SEQ ID 113; cp7132>:

```
    1 MCNSIAMKKQ KRGFVLMELL MSFTLIALLL GTLGFWYRKI YTVQKQKERI
  51 YNFYIEESRA YKQLRTLFSM SLSSSYEEPG SLFSLIFDRG VYRDPKLAGA
101 VRASLHHDTK DQRLELRICN IKDQSYFETQ RLLSHVTHVV LSFQRNPDPE
151 KLPETIALTI TREPKAYPPR TLTYQFAVGK*
```

A predicted signal peptide is highlighted.

The cp7132 nucleotide sequence <SEQ ID 114> is:

```
    1 ATGTGTAACT CTATAGCTAT GAAAAAGCAA AAGCGTGGCT TTGTGCTTAT
  51 GGAATTACTC ATGTCGTTCA CTCTAATTGC TTTGTTATTA GGGACTTTAG
101 GATTTTGGTA TCGGAAAATT TATACTGTAC AAAAGCAAAA AGAACGTATT
151 TATAACTTTT ATATCGAAGA AAGCCGAGCC TACAAGCAGC TCAGAACCCT
201 GTTTAGCATG TCCTTGTCTT CATCTTACGA GGAGCCTGGA TCATTATTTT
251 CTTTAATCTT TGATCGGGGT GTTTATCGAG ATCCTAAGCT GGCAGGTGCG
301 GTACGAGCTT CTCTCCATCA TGACACCAAG GATCAGAGAT TGGAACTTCG
351 TATTTGTAAT ATTAAGGATC AGTCTTACTT TGAAACACAG CGACTGCTCT
401 CCCACGTGAC CCATGTTGTA CTTTCCTTCC AGAGAAATCC TGATCCTGAA
451 AAACTTCCTG AAACAATTGC TTTAACTATA ACACGGGAAC CTAAAGCATA
501 TCCTCCAAGG ACGTTAACAT ACCAATTTGC GGTTGGGAAA TAA
```

The PSORT algorithm predicts a periplasmic location (0.915).

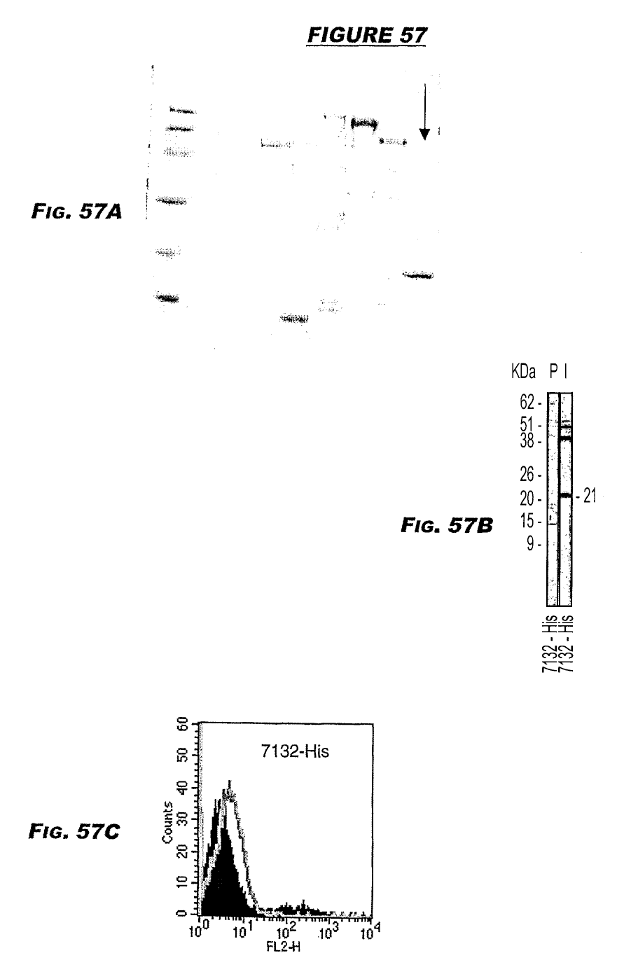

The protein was expressed in E. coli and purified as a his-tag product (FIG. 57A) or as a GST-fusion. The recombinant proteins were used to immunize mice, whose sera were used in a Western blot (FIG. 57B) and FACS (FIG. 57C) analyses.

These experiments show that cp7132 is a surface-exposed and immunoaccessible protein, and that it is a useful immunogen. These properties are not evident from the sequence alone.

### Example 58

The following C. pneumoniae protein (PID 4376733) was expressed <SEQ ID 115; cp6733>:

```
    1 MKTSIPWVLV SSVLAFSCHL QSLANEELLS PDDSFNGNID SGTFTPKTSA
  51 TTYSLTGDVF FYEPGKGTPL SDSCFKQTTD NLTFLGNGHS LTFGFIDAGT
101 HAGAAASTTA NKNLTFSGFS LLSFDSSPST TVTTGQGTLS SAGGVNLENI
151 RKLVVAGNFS TADGGATKGA SFLLTGTSGD ALFSNNSSST KGGAIATTAG
201 ARIANNTGYV RFLSNIASTS GGAIDDEGTS ILSNNKFLYF EGNAAKTTGG
251 AICNTKASGS PELIISNNKT LIFASNVAET SGGAIHAKKL ALSSGGFTEF
301 LRNNVSSATP KGGAISIDAS GELSLSAETG NITFVRNTLT TTGSTDTPKR
351 NAINIGSNGK FTELPAAKNH TIFFYDPITS EGTSSDVLKI NNGSAGALNP
401 YQGTILFSGE TLTADELKVA DNLKSSFTQP VSLSGGKLLL QKGVTLESTS
451 FSQEAGSLLG MDSGTTLSTT AGSITITNLG INVDSLGLKQ PVSLTAKGAS
501 NKVIVSGKLN LIDIEGNIYE SHMFSHDQLF SLLKITVDAD VDTNVDTSSL
551 IPVPAEDPNS EYGFQGQWNV NWTTDTATNT KEATATWTKT GFVPSPERKS
601 ALVCNTLWGV FTDIRSLQQL VEIGATGMEH KQGFWVSSMT NFLHKTGDEN
651 RKGFRHTSGG YVIGGSAHTP KDDLFTFAFC HLFARDKDCF IAHNNSRTYG
701 GTLFFKHSHT LQPQNYLRLG RAKFSESAIE KEPREIPLAL DVQVSFSHSD
751 NRMETHYTSL PESEGSWSNE CIAGGIGLDL PFVLSNPHPL FKTFIPQMKV
801 EMVYVSQNSF FESSSDGRGF SIGRLLNLSI PVGAKFVQGD IGDSYTYDLS
851 GFFVSDVYRN NPQSTATLVM SPDSWKTRGG NLSRQAFLLR GSNNYVYNSN
901 CELFGHYAME LRGSSRNYNV DVGTKLRF*
```

A predicted signal peptide is highlighted.

The cp6733 nucleotide sequence <SEQ ID 116> is:

```
      1 ATGAAGACTT CGATTCCTTG GGTTTTAGTT TCCTCCGTGT TAGCTTTCTC
    51 ATGTCACCTA CAGTCACTAG CTAACGAGGA ACTTTTATCA CCTGATGATA
  101 GCTTTAATGG AAATATCGAT TCAGGAACGT TTACTCCAAA AACTTCAGCC
  151 ACAACATATT CTCTAACAGG AGATGTCTTC TTTTACGAGC CTGGAAAAGG
  201 CACTCCCTTA TCTGACAGTT GTTTTAAGCA AACCACGGAC AATCTTACCT
  251 TCTTGGGGAA CGGTCATAGC TTAACGTTTG GCTTTATAGA TGCTGGCACT
  301 CATGCAGGTG CTGCTGCATC TACAACAGCA AATAAGAATC TTACCTTCTC
  351 AGGGTTTTCC TTACTGAGTT TTGATTCCTC TCCTAGCACA ACGGTTACTA
  401 CAGGTCAGGG AACGCTTTCC TCAGCAGGAG GCGTAAATTT AGAAAATATT
  451 CGTAAACTTG TAGTTGCTGG GAATTTTTCT ACTGCAGATG GTGGAGCTAT
  501 CAAAGGAGCG TCTTTCCTTT TAACTGGCAC TTCTGGAGAT GCTCTTTTTA
  551 GTAACAACTC TTCATCAACA AAGGGAGGAG CAATTGCTAC TACAGCAGGC
  601 GCTCGCATAG CAAATAACAC AGGTTATGTT AGATTCCTAT CTAACATAGC
  651 GTCTACGTCA GGAGGCGCTA TCGATGATGA AGGCACGTCG ATACTATCGA
  701 ACAACAAATT TCTATATTTT GAAGGGAATG CAGCGAAAAC TACTGGCGGT
  751 GCGATCTGCA ACACCAAGGC GAGTGGATCT CCTGAACTGA TAATCTCTAA
  801 CAATAAGACT CTGATCTTTG CTTCAAACGT AGCAGAAACA AGCGGTGGCG
  851 CCATCCATGC TAAAAAGCTA GCCCTTTCCT CTGGAGGCTT TACAGAGTTT
  901 CTACGAAATA ATGTCTCATC AGCAACTCCT AAGGGGGGTG CTATCAGCAT
  951 CGATGCCTCA GGAGAGCTCA GTCTTTCTGC AGAGACAGGA AACATTACCT
1001 TTGTAAGAAA TACCCTTACA ACAACCGGAA GTACCGATAC TCCTAAACGT
1051 AATGCGATCA ACATAGGAAG TAACGGGAAA TTCACGGAAT TACGGGCTGC
1101 TAAAAATCAT ACAATTTTCT TCTATGATCC CATCACTTCA GAAGGAACCT
1151 CATCAGACGT ATTGAAGATA AATAACGGCT CTGCGGGAGC TCTCAATCCA
1201 TATCAAGGAA CGATTCTATT TTCTGGAGAA ACCCTAACAG CAGATGAACT
1251 TAAAGTTGCT GACAATTTAA AATCTTCATT CACGCAGCCA GTCTCCCTAT
1301 CCGGAGGAAA GTTATTGCTA CAAAAGGGAG TCACTTTAGA GAGCACGAGC
1351 TTCTCTCAAG AGGCCGGTTC TCTCCTCGGC ATGGATTCAG GAACGACATT
1401 ATCAACTACA GCTGGGAGTA TTACAATCAC GAACCTAGGA ATCAATGTTG
1451 ACTCCTTAGG TCTTAAGCAG CCCGTCAGCC TAACAGCAAA AGGTGCTTCA
1501 AATAAAGTGA TCGTATCTGG GAAGCTCAAC CTGATTGATA TTGAAGGGAA
1551 CATTTATGAA AGTCATATGT TCAGCCATGA CCAGCTCTTC TCTCTATTAA
1601 AAATCACGGT TGATGCTGAT GTTGATACTA ACGTTGACAT CAGCAGCCTT
1651 ATCCCTGTTC CTGCTGAGGA TCCTAATTCA GAATACGGAT TCCAAGGACA
1701 ATGGAATGTT AATTGGACTA CGGATACAGC TACAAATACA AAAGAGGCCA
1751 CGGCAACTTG GACCAAAACA GGATTTGTTC CCAGCCCCGA AAGAAAATCT
1801 GCGTTAGTAT GCAATACCCT ATGGGGAGTC TTTACTGACA TTCGCTCTCT
1851 GCAACAGCTT GTAGAGATCG GCGCAACTGG TATGGAACAC AAACAAGGTT
1901 TCTGGGTTTC CTCCATGACG AACTTCCTGC ATAAGACTGG AGATGAAAAT
1951 CGCAAAGGCT TCCGTCATAC CTCTGGAGGC TACGTCATCG GTGGAAGTGC
2001 TCACACTCCT AAAGACGACC TATTTACCTT TGCGTTCTGC CATCTCTTTG
2051 CTAGAGACAA AGATTGTTTT ATCGCTCACA ACAACTCTAG AACCTACGGT
2101 GGAACTTTAT TCTTCAAGCA CTCTCATACC CTACAACCCC AAAACTATTT
2151 GAGATTAGGA AGAGCAAAGT TTTCTGAATC AGCTATAGAA AAATTCCCTA
2201 GGGAAATTCC CCTAGCCTTG GATGTCCAAG TTTCGTTCAG CCATTCAGAC
2251 AACCGTATGG AAACGCACTA TACCTCATTG CCAGAATCCG AAGGTTCTTG
2301 GAGCAACGAG TGTATAGCTG GTGGTATCGG CCTAGACCTT CCTTTTGTTC
2351 TTTCCAACCC ACATCCTCTT TTCAAGACCT TCATTCCACA GATGAAAGTC
2401 GAAATGGTTT ATGTATCACA AAATAGCTTC TTCGAAAGCT CTAGTGATGG
2451 CCGTGGTTTT AGTATTGGAA GGCTGCTTAA CCTCTCGATT CCTGTGGGTG
2501 CGAAATTCGT GCAGGGGGAT ATCGGAGATT CCTACACCTA TGATCTCTCA
2551 GGATTCTTTG TTTCCGATGT CTATCGTAAC AATCCCCAAT CTACAGCGAC
2601 TCTTGTGATG AGCCCAGACT CTTGGAAAAT TCGCGGTGGC AATCTTTCAA
2651 GACAGGCATT TTTACTGAGG GGTAGCAACA ACTACGTCTA CAACTCCAAT
2701 TGTGAGCTCT TCGGACATTA CGCTATGGAA CTCCGTGGAT CTTCAAGGAA
2751 CTACAATGTA GATGTTGGTA CCAAACTCCG ATTCTAG
```

The PSORT algorithm predicts an outer membrane location (0.924).

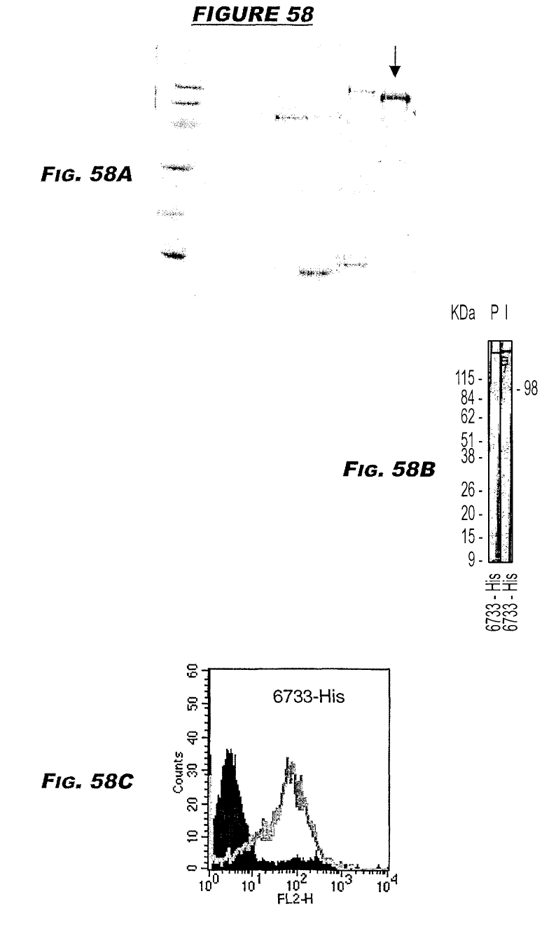

The protein was expressed in E. coli and purified as a his-tag product, as shown in FIG. 58A. The recombinant protein was used to immunize mice, whose sera were used in a Western blot (FIG. 58B) and for FACS (FIG. 58C) analyses. A GST-fusion protein was also expressed.

The cp6733 protein was also identified in the 2D-PAGE experiment (Cpn0451).

These experiments show that cp6733 is a surface-exposed and immunoaccessible protein, and that it is a useful immunogen. These properties are not evident from the sequence alone.

### Example 59

The following C. pneumoniae protein (PID 4376814) was expressed <SEQ ID 117; cp6814>:

```
    1 MHDALLSILA IQELDIKMIR LMRVKKEHQK ELAKVQSLKS DIRRKVQEKE
  51 LEMENLKTQI RDGENRIQEI SEQINKLENQ QAAVKKMDEF NALTQEMTTA
101 NKERRSLEHQ LSDLMDKQAG GEDLIVSLKE SLASTENSSS VIEKEIFESI
151 KKINEEGKAL LEQRTELKHA TNPELLSIYE RLLNNKKDRV VVPIENRVCS
201 GCHIVLTPQH ENLVRKKDRL IFCEHCSRIL YWQESQVNAQ ENSTAKRRRR
251 RAAV*
```

The cp6814 nucleotide sequence <SEQ ID 118> is:

```
    1 ATGCATGACG CACTTCTAAG CATTTTGGCT ATTCAAGAGC TTGATATTAA
  51 AATGATTCGC CTTATGCGCG TAAAGAAAGA ACATCAGAAA GAATTGGCTA
101 AAGTCCAATC TTTAAAAAGT GATATTCGTA GAAAAGTTCA GGAAAAAGAA
151 CTCGAAATGG AGAATTTGAA AACTCAAATT CGAGATGGAG AGAATCGCAT
201 CCAAGAGATT TCTGAACAAA TCAATAAATT AGAAAATCAG CAAGCTGCTG
251 TAAAAAAAAT GGATGAGTTT AACGCTCTTA CCCAAGAAAT GACTACAGCA
301 AACAAAGAAC GTCGCTCTTT AGAGCACCAG CTTAGCGATC TCATGGATAA
351 GCAAGCTGGA GGCGAAGACC TTATTGTCTC TCTAAAAGAA AGCTTAGCTT
401 CTACAGAAAA TAGTAGCAGT GTCATTGAAA AAGAAATTTT TGAAAGCATC
451 AAAAAGATTA ATGAAGAAGG CAAAGCTTTG CTTGAACAAC GGACAGAGTT
501 AAAGCATGCG ACGAATCCCG AACTACTCAG CATCTATGAG CGTCTATTAA
551 ACAATAAAAA AGATCGCGTT GTTGTTCCTA TTGAAAATCG TGTCTGCAGT
601 GGTTGTCATA TTGTTCTAAC TCCTCAACAC GAAAATCTTG TAAGAAAGAA
651 AGACCGACTC ATTTTTTGCG AACATTGCTC TCGAATTCTC TATTGGCAAG
701 AATCCCAAGT CAATGCTCAG GAAAATTCCA CAGCAAAACG TCGTCGTCGT
751 CGCGCAGCTG TATAA
```

The PSORT algorithm predicts an inner membrane location (0.070).

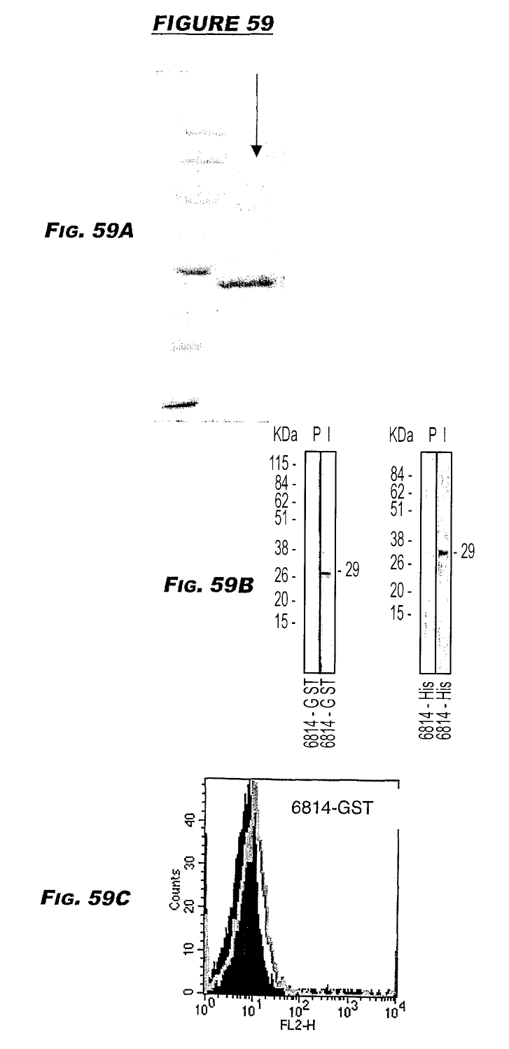

The protein was expressed in E. coli and purified as a GST-fusion (FIG. 59A) or his-tagged product. The recombinant proteins were used to immunize mice, whose sera were used in Western blot (FIG. 59B) and FACS (FIG. 59C) analyses.

These experiments show that cp6814 is a surface-exposed and immunoaccessible protein, and that it is a useful immunogen. These properties are not evident from the sequence alone.

### Example 60

The following C. pneumoniae protein (PID 4376830) was expressed <SEQ ID 119; cp6830>:

```
      1 MKWLPATAVF AAVLPALTAF GDPASVEIST SHTGSGDPTS DAALTGFTQS
    51 STETDGTTYT IVGDITFSTF TNIPVPVVTP DANDSSSNSS KGGSSSSGAT
  101 SLIRSSNLHS DFDFTKDSVL DLYHLFFPSA SNTLNPALLS SSSSGGSSSS
  151 SSSSSSGSAS AVVAADPKGG AAFYSNEANG TLTFTTDSGN PGSLTLQNLK
  201 MTGDGAAIYS KGPLVFTGLK NLTFTGNESQ KSGGAAYTEG ALTTQAIVEA
  251 VTFTGNTSAG QGGAIYVKEA TLFNALDSLK FEKNTSGQAG GGIYTESTLT
  301 ISNITKSIEF ISNKASVPAP APEPTSPAPS SITNSTTIDT STLQTPAASA
  351 TPAVAPVAAV TPTPISTQET AGNGGATYAK QGISISTFKD LTFKSNSASV
  401 DATLTVDSST IGESGGAIFA ADSIQIQQCT GTTLFSGNTA NKSGGGIYAV
  451 GQVTLEDIAN LKMTNNTCKG EGGAIYTKKA LTINNGAILT TFSGNTSTDN
  501 GGAIFAVGGI TLSDLVEVRF SKNKTGNYSA PITKAASNTA PVVSSSTTAA
  551 SPAVPAAAAA PVTNAAKGGA LYSTEGLTVS GITSILSFEN NECQNQGGGA
  601 YVTKTFQCSD SHRLQFTSNK AADEGGGLYC GDDVTLTNLT GKTLFQENSS
  651 EKHGGGLSLA SGKSLTMTSL ESFCLNANTA KENGGGANVP ENIVLTFTYT
  701 PTPNEPAPVQ QPVYGEALVT GNTATKSGGG IYTKNAAFSN LSSVTFDQNT
  751 SSENGGALLT QKAADKTDCS FTYITNVNIT NNTATGNGGG IAGGKAHFDR
  801 IDNLTVQSNQ AKKGGGVYLE DALILEKVIT GSVSQNTATE SGGGIYAKDI
  851 QLQALPGSFT ITDNKVETSL TTSTNLYGGG IYSSGAVTLT NISGTFGITG
  901 NSVINTATSQ DADIQGGGTY ATTSLSINQC NTPILFSNNS AATKKTSTTK
  951 QIAGGAIFSA AVTIENNSQP IIFLNNSAKS EATTAATAGN KDSCGGAIAA
1001 NSVTLTNNPE ITFKGNYAET GGAIGCIDLT NGSPPRKVSI ADNGSVLFQD
1051 NSALNRGGAI YGETIDISRT GATFIGNSSK HDGSAICCST ALTLAPNSQL
1101 IFENNKVTET TATTKASINN LGAAIYGNNE TSDVTISLSA ENGSIFFKNN
1151 LCTATNKYCS IAGNVKFTAI EASAGKAISF YDAVNVSTKE TNAQELKLNE
1201 KATSTGTILF SGELHENKSY IPQKVTFAHG NLILGKNAEL SVVSFTQSPG
1251 TTITMGPGSV LSNHSKEAGG IAINNIITDF SEIVPTKDNA TVAPPTLKLV
1301 SRTNADSKDK IDITGTVTLL DPNGNLYQNS YLGEDRDITL FNIDNSASGA
1351 VTATNVTLQG NLGAKKGYLG TWNLDPNSSG SKIILKWTFD KYLRWPYIPR
1401 DNHFYINSIW GAQNSLVTVK QGILGNMLNN ARFEDPAFNN FWASAIGSFL
1451 RKEVSRNSDS FTYHGRGYTA AVDAKPRQEF ILGAAFSQVF GHAESEYHLD
1501 NYKHKGSGHS TQASLYAGNI FYFPAIRSRP ILFQGVATYG YMQHDTTTYY
1551 PSIEEKNMAN WDSIAWLFDL RFSVDLKEPQ PHSTARLTFY TEAEYTRIRQ
1601 EKFTELDYDE RSFSACSYGN LAIPTGFSVD GALAWREIIL YNKVSAAYLP
1651 VILRNNPKAT YEVLSTKEKG NVVNVLPTRN AARAEVSSQI YLGSYWTLYG
1701 TYTIDASMNT LVQMANGGIR FVF*
```

A predicted signal peptide is highlighted.

The cp6830 nucleotide sequence <SEQ ID 120> is:

```
      1 ATGAAGTGGC TACCAGCTAC AGCTGTTTTT GCTGCCGTAC TCCCCGCACT
    51 AACAGCCTTC GGAGATCCCG CGTCTGTTGA AATAAGTACC AGCCATACAG
  101 GATCCGGGGA TCCTACAAGC GACGCTGCCT TAACAGGATT TACACAAAGT
  151 TCCACAGAAA CTGACGGTAC TACCTATACC ATTGTCGGTG ATATCACCTT
  201 CTCTACTTTT ACGAATATTC CTGTTCCCGT AGTAACTCCA GACGCCAACG
  251 ATAGTTCCAG CAATAGCTCT AAAGGAGGAA GTAGCAGTAG TGGAGCTACA
  301 TCTCTAATCC GATCCTCAAA CCTACACTCC GATTTTGATT TTACAAAAGA
  351 TAGCGTGTTA GACCTCTATC ACCTTTTCTT TCCTTCAGCT TCAAATACTC
  401 TCAATCCTGC ACTCCTTTCT TCCAGTAGCA GCGGTGGATC CTCGAGCAGC
  451 AGTAGCTCCT CATCATCTGG AAGTGCATCT GCTGTTGTTG CTGCGGACCC
  501 AAAAGGAGGC GCTGCCTTTT ATAGTAACGA GGCTAACGGA ACTTTAACCT
  551 TCACTACAGA CTCTGGAAAT CCCGGCTCCC TGACTCTTCA GAATCTTAAA
  601 ATGACCGGAG ATGGAGCCGC CATCTACTCG AAGGGTCCTC TAGTATTTAC
  651 TGGTTTAAAA AATCTAACCT TTACAGGAAA TGAATCTCAG AAATCTGGAG
  701 GTGCTGCCTA TACTGAAGGC GCACTCACAA CACAAGCAAT CGTTGAAGCC
  751 GTAACTTTTA CTGGCAACAC CTCGGCAGGG CAAGGAGGCG CTATCTATGT
  801 TAAAGAAGCT ACCCTATTCA ATGCTCTAGA CAGCCTCAAA TTTGAAAAAA
  851 ACACTTCTGG GCAAGCTGGT GGTGGAATCT ATACAGAGTC TACGCTCACA
  901 ATCTCGAACA TCACAAAATC TATTGAATTT ATCTCTAATA AAGCTTCTGT
  951 CCCTGCCCCC GCTCCTGAGC CCACCTCTCC GGCTCCAAGT AGCTTAATAA
1001 ATTCTACAAC GATCGATACC TCGACTCTCC AAACCCGAGC AGCATCCGCA
1051 ACTCCAGCAG TGGCTCCTGT TGCTGCCGTA ACTCCAACAC CAATCTCTAC
1101 TCAAGAGACC GCAGGAAATG GAGGCGCTAT CTATGCTAAA CAAGGTATTT
1151 CGATATCCAC GTTTAAAGAT CTGACCTTCA AGTCTAACTC TGCATCGGTA
1201 GATGCCACCC TTACTGTCGA TTCTAGCACT ATTGGAGAAT CTGGAGGTGC
1251 TATCTTTGCA GCAGACTCTA TACAAATCCA ACAGTGCACG GGAACCACCT
1301 TATTCAGTGG CAATACTGCC AATAAGTCTG GTGGGGGTAT TTACGCTGTA
1351 GGACAAGTCA CCCTAGAAGA TATAGCGAAT CTGAAGATGA CCAACAACAC
1401 CTGTAAAGGT GAAGGTGGAG CCATCTACAC TAAAAAGGCT TTAACTATCA
1451 ACAACGGTGC CATTCTCACT ACATTTTCTG GAAATACATC GACAGATAAT
1501 GGTGGGGCTA TTTTTGCTGT AGGTGGCATC ACTCTCTCTG ATCTTGTAGA
1551 AGTCCGCTTT AGTAAAAATA AGACCGGAAA TTATTCCGCT CCTATTACCA
1601 AAGCGGCTAG CAACACAGCT CCTGTAGTTT CTAGCTCTAC AACTGCTGCA
1651 TCTCCTGCGG TCCCTGCTGC CGCTGCAGCA CCTGTTACAA ACGCAGCAAA
1701 AGGAGGGGCT TTATATAGTA CAGAAGGACT GACTGTATCT GGAATCACAT
1751 CGATATTGTC GTTTGAAAAC AACGAATGCC AGAATCAAGG AGGTGGGGCT
1801 TACGTTACTA AAACCTTCCA GTGTTCCGAT TCTCATCGCC TCCAGTTTAC
1851 TAGTAATAAA GCAGCAGATG AAGGCGGGGG CCTGTATTGT GGTGACGATG
1901 TCACGCTAAC GAACCTGACA GGGAAAACAC TATTTCAAGA GAATAGCAGT
1951 GAGAAACATG GAGGTGGGCT CTCTCTCGCC TCAGGAAAAT CTCTGACTAT
2001 GACATCGTTA GAGAGCTTCT GCTTAAATGC AAATACAGCA AAGGAAAACG
2051 GAGGCGGTGC GAATGTCCCT GAAAATATTG TACTCACCTT CACCTATACT
2101 CCCACTCCAA ATGAACCTGC GCCTGTGCAG CAGCCCGTGT ATGGAGAAGC
2151 TCTTGTTACT GGAAATACAG CCACAAAAAG TGGTGGGGGC ATTTACACGA
2201 AAAATGCGGC CTTCTCAAAT TTATCTTCTG TAACTTTTGA TCAAAATACC
2251 TCTTCAGAAA ATGGTGGTGC CTTACTTACC CAAAAAGCTG CAGATAAAAC
2301 GGACTGTTCT TTCACCTATA TTACAAATGT CAATATCACC AACAATACAG
2351 CTACAGGAAA TGGTGGGGGC ATTGCTGGGG GAAAAGCACA TTTCGATCGC
2401 ATTGATAATC TTACAGTCCA AAGCAACCAA GCAAAGAAAG GTGGTGGGGT
2451 TTATCTTGAA GATGCCCTCA TCCTGGAAAA GGTTATTACA GGTTCTGTCT
2501 CACAAAATAC AGCTACAGAA AGTGGTGGGG GTATCTACGC TAAGGATATT
2551 CAACTACAAG CTCTACCTGG AAGCTTCACA ATTACCGATA ATAAAGTCGA
2601 AACTAGTCTT ACTACTAGCA CTAATTTATA TGGTGGGGGC ATCTATTCCA
2651 GTGGAGCTGT CACGCTAACC AATATATCTG GAACCTTTGG CATTACAGGA
2701 AACTCTGTTA TCAATACAGC GACATCCCAG GATGCAGATA TACAAGGTGG
2751 GGGCATTTAT GCAACCACGT CTCTCTCAAT AAATCAATGT AATACACCCA
2801 TTCTATTTAG CAACAACTCT GCTGCCACTA AAAAAACATC AACAACAAAG
2851 CAAATTGCTG GTGGGGCTAT CTTCTCCGCT GCAGTAACTA TCGAGAATAA
2901 CTCTCAGCCC ATTATTTTCT TAAATAATTC CGCAAAGTCG GAAGCAACTA
2951 CAGCAGCAAC TGCAGGAAAT AAAGATAGCT GTGGAGGAGC CATTGCAGCT
3001 AACTCTGTTA CTTTAACAAA TAACCCTGAA ATAACCTTTA AAGGAAATTA
3051 TGCAGAAACT GGAGGAGCGA TTGGCTGTAT TGATCTTACT AATGGCTCAC
3101 CTCCCCGTAA AGTCTCTATT GCAGACAACG GTTCTGTCCT TTTTCAAGAC
3151 AACTCTGCGT TAAATCGCGG AGGCGCTATC TATGGAGAGA CTATCGATAT
3201 CTCCAGGACA GGTGCGACTT TCATCGGTAA CTCTTCAAAA CATGATGGAA
3251 GTGCAATTTG CTGTTCAACA GCCCTAACTC TTGCGCCAAA CTCCCAACTT
3301 ATCTTTGAAA ACAATAAGGT TACGGAAACC ACAGCCACTA CAAAAGCTTC
3351 CATAAATAAT TTAGGAGCTG CAATTTATGG AAATAATGAG ACTAGTGACG
3401 TCACTATCTC TTTATCAGCT GAGAATGGAA GTATTTTCTT TAAAAACAAT
3451 CTATGCACAG CAACAAACAA ATACTGCAGT ATTGCTGGAA ACGTAAAATT
3501 TACAGCAATA GAAGCTTCAG CAGGGAAAGC TATATCTTTC TATGATGCAG
3551 TTAACGTTTC CACCAAAGAA ACAAATGCTC AAGAGCTAAA ATTAAATGAA
3601 AAAGCGACAA GTACAGGAAC GATTCTATTT TCTGGGGAAC TTCACGAAAA
3651 TAAATCCTAT ATTCCACAGA AAGTCACTTT CGCACATGGG AATCTCATTC
3701 TAGGTAAAAA TGCAGAACTT AGCGTAGTTT CCTTTACCCA ATCTCCAGGC
3751 ACCACAATCA CTATGGGCCC AGGATCGGTT CTTTCCAACC ATAGCAAAGA
3801 AGCAGGAGGA ATCGCTATAA ACAATGTCAT CATTGATTTT AGTGAAATCG
3851 TTCCTACTAA AGATAATGCA ACAGTAGCTC CACCCACTCT TAAATTAGTA
3901 TCGAGAACTA ATGCAGATAG TAAAGATAAG ATTGATATTA CAGGAACTGT
3951 GACTCTTCTA GATCCTAATG GCAACTTATA TCAAAATTCT TATCTTGGTG
4001 AAGACCGCGA TATCACTCTT TTCAATATAG ACAATTCTGC AAGTGGGGCA
4051 GTTACAGCCA CGAATGTCAC CCTTCAAGGG AATTTAGGAG CTAAAAAAGG
4101 ATATTTAGGA ACCTGGAATT TGGATCCAAA TTCCTCGGGT TCAAAAATTA
4151 TTCTAAAATG GACCTTTGAC AAATACCTGC GCTGGCCCTA CATCCCTAGA
4201 GACAACCACT TCTACATCAA CTCTATTTGG GGAGCACAAA ACTCTTTAGT
4251 GACTGTGAAA CAAGGGATCT TAGGGAACAT GTTGAACAAT GCAAGGTTTG
4301 AAGATCCTGC TTTCAACAAC TTCTGGGCTT CGGCTATAGG ATCTTTCCTT
4351 AGGAAAGAAG TATCTCGAAA TTCTGACTCA TTCACCTATC ATGGCAGAGG
4401 CTATACCGCT GCTGTGGATG CCAAACCTCG CCAAGAATTT ATTTTAGGAG
4451 CTGCCTTCAG TCAGGTTTTT GGTCACGCCG AGTCTGAATA TCACCTTGAC
4501 AACTATAAGC ATAAAGGCTC AGGTCACTCT ACACAAGCAT CTCTTTATGC
4551 TGGCAATATC TTCTATTTTC CTGCGATACG GTCTCGGCCT ATTCTATTCC
4601 AAGGTGTGGC GACCTATGGT TATATGCAAC ATGACACCAC AACCTACTAT
4651 CCTTCTATTG AAGAAAAAAA TATGGCAAAC TGGGATAGCA TTGCTTGGTT
4701 ATTTGATCTG CGTTTCAGTG TGGATCTTAA AGAACCTCAA CCTCACTCTA
4751 CAGCAAGGCT TACCTTCTAT ACAGAAGCTG AGTATACCAG AATTCGCCAG
4801 GAGAAATTCA CAGAGCTAGA CTATGATCCT AGATCTTTCT CTGCATGCTC
4851 TTATGGAAAC TTAGCAATTC CTACTGGATT CTCTGTAGAC GGAGCATTAG
4901 CTTGGCGTGA GATTATTCTA TATAATAAAG TATCAGCTGC GTACCTCCCT
4951 GTGATTCTCA GGAATAATCC AAAAGCGACC TATGAAGTTC TCTCTACAAA
5001 AGAAAAGGGC AACGTAGTCA ACGTTCTCCC TACAAGAAAC GCAGCTCGTG
5051 CAGAGGTGAG CTCTCAAATT TATCTTGGAA GTTACTGGAC ACTCTACGGC
5101 ACGTATACTA TTGATGCTTC AATGAATACT TTAGTGCAAA TGGCCAACGG
5151 AGGGATCCGG TTTGTATTCT AG
```

The PSORT algorithm predicts an outer membrane location (0.926).

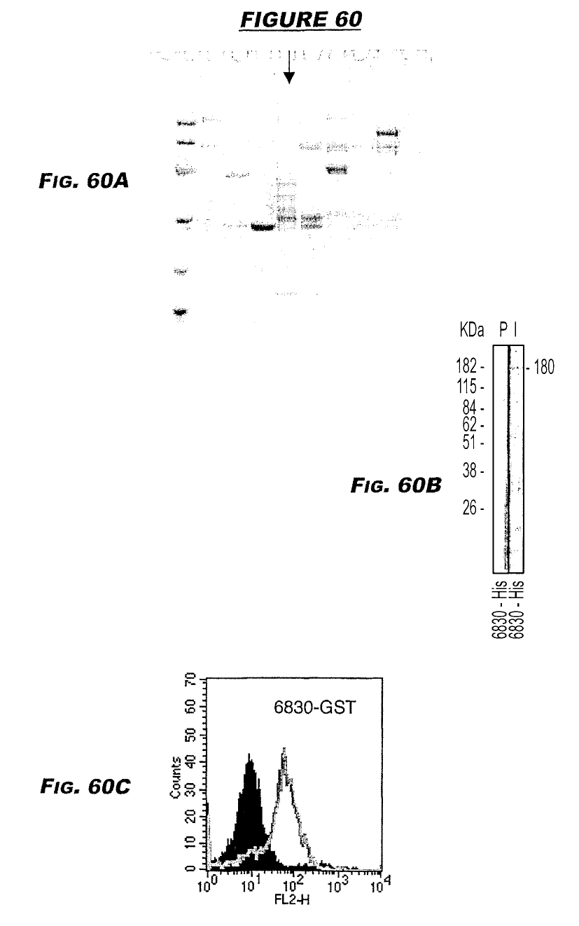

The protein was expressed in E. coli and purified as a GST-fusion (FIG. 60A) or his-tagged product. The recombinant proteins were used to immunize mice, whose sera were used in Western blot (FIG. 60B) and FACS (FIG. 60C) analyses.

The cp6830 protein was also identified in the 2D-PAGE experiment (Cpn0540) and showed good cross-reactivity with human sera, including sera from patients with pneumonitis.

These experiments show that cp6830 is a surface-exposed and immunoaccessible protein, and that it is a useful immunogen. These properties are not evident from the sequence alone.

### Example 61

The following C. pneumoniae protein (PID 4376854) was expressed <SEQ ID 121; cp6854>:

```
    1 MSIAIAREQY AAILDMHPKP SIAMESSEQA RTSWEKRQAH PYLYRLLEII
  51 WGVVKFLLGL IFFIPLGLFW VLQKICQNFI LLGAGGWIFR PICRDSNLLR
101 QAYAARLFSA SFQDHVSSVR RVCLQYDEVF IDGLELRLPN AKPDRWMLIS
151 NGNSDCLEYR TVLQGEKDWI FRIAEESQSN ILIFNYPGVM KSQGNITRNN
201 VVKSYQACVR YLRDEPAGPQ ARQIVAYGYS LGASVQAEAL SKEIADGSDS
251 VRWFVVKDRG ARSTGAVAKQ FIGSLGVWLA NLTHWNINSE KRSKDLHCPE
301 LFIYGKDSQG NLIGDGLFKK ETCFAAPFLD PKNLEECSGK KIPVAQTGLR
351 HDHILSDDVI KEVAGHIQRH FDN*
```

The cp6854 nucleotide sequence <SEQ ID 122> is:

```
      1 ATGTCAATAG CTATTGCAAG GGAACAATAC GCAGCTATAT TGGATATGCA
    51 TCCTAAACCT TCGATCGCCA TGTTTTCTTC GGAGCAGGCG AGAACTTCTT
  101 GGGAGAAACG ACAGGCTCAT CCTTACCTTT ATCGTCTTCT TGAGATCATA
  151 TGGGGTGTTG TGAAATTTCT TCTCGGCTTA ATCTTCTTTA TTCCCTTGGG
  201 TCTTTTCTGG GTCCTTCAGA AGATATGTCA GAATTTTATT CTTCTTGGTG
  251 CAGGAGGGTG GATTTTTAGA CCCATATGCA GGGACTCTAA TTTATTGCGA
  301 CAAGCTTACG CCGCGCGTCT TTTCTCCGCT TCATTCCAAG ATCATGTCTC
  351 CTCTGTGCGA AGGGTTTGCT TACAGTATGA CGAGGTCTTT ATTGACGGAT
  401 TGGAGTTACG TCTTCCCAAT GCTAAGCCAG ATCGATGGAT GTTAATCTCC
  451 AATGGAAACT CCGATTGCTT AGAGTATAGG ACAGTGCTGC AAGGGGAAAA
  501 GGACTGGATA TTCCGTATTG CTGAAGAGTC TCAATCCAAC ATTTTAATCT
  551 TCAATTACCC AGGAGTCATG AAGAGCCAAG GGAATATAAC AAGAAACAAT
  601 GTAGTCAAAT CTTATCAAGC ATGCGTACGC TATCTTAGAG ATGAACCCGC
  651 AGGACCTCAG GCGCGTCAAA TCGTTGCTTA TGGCTATTCT TTAGGAGCTA
  701 GTGTTCAAGC CGAAGCATTA AGTAAAGAGA TCGCAGACGG AAGTGATAGC
  751 GTCCGTTGGT TTGTCGTTAA AGATCGAGGA GCTCGCTCTA CAGGAGCCGT
  801 TGCTAAACAG TTTATTGGAA GTCTAGGAGT TTGGCTGGCG AATCTTACCC
  851 ATTGGAATAT TAATTCTGAA AAGAGAAGCA AGGACTTGCA TTGCCCAGAA
  901 CTCTTTATTT ATGGCAAGGA TTCCCAAGGT AATCTTATCG GGGATGGATT
  951 GTTCAAAAAA GAGACGTGCT TCGCAGCACC ATTTTTAGAT CCTAAAAACT
1001 TGGAAGAGTG TTCAGGGAAG AAAATCCCTG TAGCTCAGAC CGGTCTAAGA
1051 CACGATCATA TCCTTTCCGA TGATGTGATT AAAGAAGTTG CAGGTCATAT
1101 TCAAAGACAT TTCGATAATT A
```

The PSORT algorithm predicts an inner membrane location (0.461).

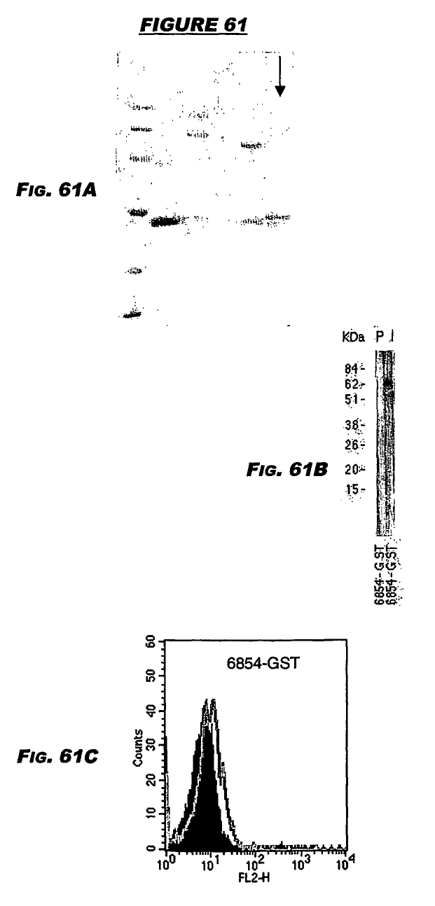

The protein was expressed in E. coli and purified as a GST-fusion product, as shown in FIG. 61A. The recombinant protein was used to immunize mice, whose sera were used in Western blot (FIG. 61B) and FACS (FIG. 61C) analyses. A his-tagged protein was also expressed.

These experiments show that cp6854 is a surface-exposed and immunoaccessible protein, and that it is a useful immunogen. These properties are not evident from the sequence alone.

### Example 62

The following C. pneumoniae protein (PID 4377101) was expressed <SEQ ID 123; cp7101>:

```
    1 MYSCYSKGIS HNYLLHPMSR LDIFVFDSLI ANQDQNLLEE IFCSEDTVLF
  51 KAYRTTALQS PLAAKNLNIA RKVANYILAD NGEIDTVKLV EAIHHLSQCT
101 YPLGPHRHNE AQDREHLLKM LKALKENPKL KESIKTLFVP SYSTIQNLIR
151 HTLALNPQTI LSTIHVRQAA LTALFTYLRQ DVGSCFATAP AILIHQEYPE
201 RFLKDLNDLI SSGKLSRIVN QREIAVPINL SGCIGELFKP LRILDLYPDP
251 LVKLSSSPGL KKAFSAANLI ETLGDSEAQI QQLLSHQYLM QKLQNVHETL
301 TANDIIKSTL LHYYQLQEST VRAIFFKEGL FSKEQVAFST QHPRELSEIQ
351 RVYHYLHAYE EAKSAFTHDT QNPLLKAWEY TLATLADASQ PTISNHIRLA
401 LGWKSEDPHS LVSLVTHFVE EEVENIRILV QQCEQTYHEA RSQLEYIEGR
451 MRNPLNNQDS QILTMDHMRF RQELNKALYE WDSAQEKAKK FLHLPEFLLS
501 FYTKQIPLYF RSSYDAFIQE FAHLYANAPA GFRILFTHGR THPNTWSPIY
551 SINEFIRELS EFFTSTESEL LGKHAVINLE KETSRLVHNI TAMLHTDVFQ
601 EALLTRILEA YQLPVPPSIL NHLDQLSQTP WVYVSGGTVD TLLLDYFESS
651 EPLTLIEKHP ENPHELAAFY ADALKDLPTG IKSYLEEGSH SLLSSSPTHV
701 FSIIAGSPLF REAWDNDWYS YTWLRDVWVK QHQDFLQDTI LPQLSIYAFI
751 ENFCNKYALQ HVVHDFHDFC SDHSLTLPEL YDKGSRFLSS LFTKDKTVAL
801 IYIRRLLYLM VREVPYVSEQ QLPEVLDNVS SYLGISSRIT YEKFRSLIEE
851 TIPKMTLLSS ADLRHIYKGL LMQSYQKIYT EEDTYLRLTT AMRHHNLAYP
901 APLLFADSNW PSIYFGFILN PGTTEIDLWK FNYAGLQGQP LDNIQELFAT
951 SRPWTLYANP IDYGMEPPEG YRSRLPKEFF *
```

The cp7101 nucleotide sequence <SEQ ID 124> is:

```
      1 ATGTATTCGT GTTACAGCAA AGGAATATCC CATAACTATC TTCTACATCC
    51 TATGTCACGT TTGGATATTT TTGTTTTCGA TTCTCTGATC GCAAACCAGG
  101 ATCAAAATCT TCTTGAGGAA ATTTTCTGTT CTGAAGACAC AGTTTTATTT
  151 AAAGCCTACC GTACTACGGC TCTACAATCC CCTCTAGCTG CTAAGAACCT
  201 AAATATCGCC CGTAAAGTCG CAAATTATAT CTTAGCTGAC AATGGGGAAA
  251 TCGATACAGT AAAGCTTGTC GAAGCCATTC ACCATCTCTC ACAATGTACC
  301 TATCCTTTAG GGCCTCATCG CCATAATGAA GCTCAAGATC GTGAACACCT
  351 CCTTAAAATG CTAAAAGCTC TAAAGGAAAA TCCTAAATTA AAAGAAAGCA
  401 TCAAAACTCT CTTTGTCCCT TCATACTCTA CAATCCAAAA CCTAATTCGC
  451 CATACACTAG CATTGAATCC ACAGACAATT CTCTCTACGA TTCATGTGCG
  501 TCAAGCAGCA CTCACAGCGC TCTTCACCTA CCTTCGGCAA GATGTAGGTT
  551 CCTGTTTTGC TACGGCTCCT GCCATTCTCA TTCACCAAGA ATATCCAGAA
  601 CGATTCCTTA AAGATCTCAA TGATCTCATT AGCAGTGGCA AACTCTCTAG
  651 AATCGTAAAC CAAAGGGAAA TTGCGGTTCC TATAAACCTT TCGGGATGCA
  701 TTGGAGAGCT ATTCAAGCCT TTAAGGATTC TAGATCTTTA TCCTGATCCT
  751 CTGGTTAAGC TCTCCTCATC TCCAGGACTC AAAAAAGCCT TTTCTGCTGC
  801 CAATCTTATT GAAACTCTTG GGGATTCTGA AGCACAAATC CAACAGTTGC
  851 TCTCGCATCA ATATTTGATG CAAAAACTAC AAAATGTCCA TGAGACCTTA
  901 ACTGCTAACG ACATTATCAA ATCGACACTT CTGCACTACT ATCAGCTCCA
  951 AGAAAGTACT GTACGAGCTA TTTTCTTCAA AGAAGGGTTG TTCAGCAAAG
1001 AACAAGTGGC ATTCTCGACG CAACACCCCA GAGAGCTCTC AGAAATACAA
1051 CGGGTATACC ACTACTTACA TGCCTATGAA GAAGCAAAAT CTGCTTTTAT
1101 CCATGACACT CAAAATCCCT TACTGAAAGC CTGGGAGTAT ACTTTAGCGA
1151 CTCTTGCGGA TGCTAGCCAA CCTACCATCT CAAACCATAT CCGCCTTGCC
1201 TTAGGATGGA AAAGTGAAGA CCCTCACAGT CTTGTATCTC TAGTTACACA
1251 CTTTGTTGAA GAGGAAGTAG AAAACATCCG AATTTTAGTC CAACAATGTG
1301 AACAGACCTA TCACGAAGCA CGCTCCCAAC TAGAATATAT TGAAGGGCGG
1351 ATGCGCAACC CACTAAATAA TCAAGACAGT CAGATTTTGA CGATGGATCA
1401 CATGCGCTTC CGTCAAGAAC TCAATAAAGC TCTTTATGAG TGGGATAGTG
1451 CTCAAGAAAA GGCAAAGAAA TTTCTACATC TTCCTGAATT CTTACTTTCT
1501 TTCTATACAA AGCAAATTCC CTTATACTTT CGTAGTTCTT ACGATGCCTT
1551 CATTCAAGAA TTTGCTCATC TCTATGCTAA TGCTCCCGCT GGCTTCCGTA
1601 TTCTTTTCAC GCATGGACGC ACCCATCCGA ACACATGGTC CCCCATCTAT
1651 TCGATTAATG AATTTATACG TTTTCTTTCT GAATTCTTCA CCTCCACAGA
1701 GTCAGAACTT CTGGGGAAAC ATGCCGTGAT CAATTTAGAG AAAGAAACAT
1751 CTCGGCTCGT CCACAACATC ACTGCCATGC TACACACGGA TGTTTTCCAA
1801 GAAGCTCTCC TTACAAGAAT TTTAGAAGCC TATCAGCTTC CTGTGCCTCC
1851 CTCCATCTTA AACCACTTAG ATCAGCTGTC ACAAACTCCC TGGGTTTATG
1901 TTTCTGGAGG AACAGTGGAC ACTCTTCTTT TGGATTATTT TGAAAGCTCA
1951 GAACCTCTGA CACTTACAGA AAAGCATCCT GAAAATCCTC ATGAGCTTGC
2001 AGCTTTCTAC GCAGACGCCC TTAAAGATCT CCCTACAGGA ATTAAAAGTT
2051 ATCTAGAAGA AGGATCCCAC TCTCTACTTA GCTCATCACC CACCCACGTT
2101 TTCTCTATAA TCGCAGGATC TCCTTTATTT CGGGAAGCTT GGGATAATGA
2151 TTGGTACAGC TATACCTGGC TTCGTGATGT CTGGGTGAAA CAACACCAAG
2201 ATTTCCTTCA AGATACTATA TTACCTCAGC TAAGTATCTA TGCTTTCATA
2251 GAGAATTTTT GTAACAAATA TGCTTTGCAA CATGTAGTTC ATGACTTTCA
2301 TGATTTCTGC TCCGACCACT CCTTGACTCT TCCGGAGCTC TATGACAAAG
2351 GATCGCGTTT TCTAAGCTCC TTATTCACCA AAGATAAGAC CGTAGCTCTT
2401 ATCTATATAC GCCGTCTTCT CTACCTTATG GTCCGTGAAG TCCCTTATGT
2451 TTCAGAACAA CAGCTTCCAG AAGTCTTAGA TAACGTCTCT TCATATCTCG
2501 GGATTTCCTC TCGTATTACC TATGAGAAAT TCCGCTCCCT GATAGAGGAA
2551 ACCATCCCTA AAATGACCTT ACTCTCCTCA GCAGACCTGA GGCATATCTA
2601 TAAAGGTCTC CTCATGCAAA GTTATCAAAA GATCTACACC GAAGAAGATA
2651 CGTACCTCCG CCTCACCACG GCAATGAGGC ATCATAATCT TGCCTATCCC
2701 GCTCCTTTGC TCTTTGCAGA CAGTAACTGG CCTTCTATTT ATTTTGGATT
2751 CATCCTAAAT CCAGGAACCA CAGAGATCGA TCTTTGGAAA TTTAACTATG
2801 CAGGGCTGCA AGGACAGCCT CTTGACAATA TCCAGGAGCT GTTCGCAACG
2851 TCAAGACCCT GGACCCTCTA TGCAAATCCT ATAGATTATG GCATGCCACC
2901 GCCTCCAGGC TACCGCAGCC GCCTCCCTAA AGAATTTTTC TAG
```

The PSORT algorithm predicts a cytoplasmic location (0.206).

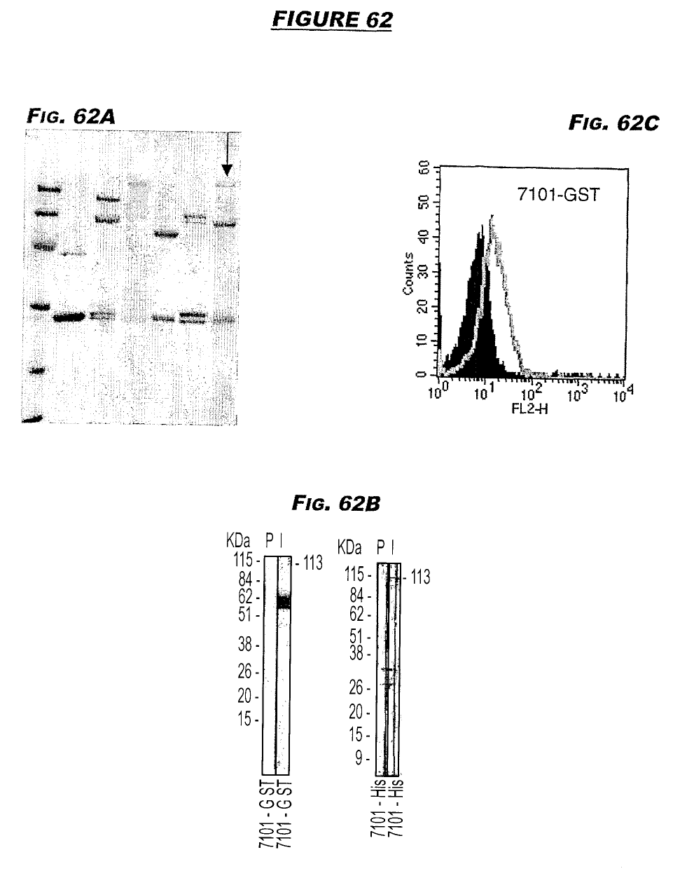

The protein was expressed in E. coli and purified as a GST-fusion (FIG. 62A) or his-tagged product. The proteins were used to immunize mice, whose sera were used in Western blot (FIG. 62B) and FACS (FIG. 62C) analyses.

This protein also showed good cross-reactivity with human sera, including sera from patients with pneumonitis.

These experiments show that cp7101 is a surface-exposed and immunoaccessible protein, and that it is a useful immunogen. These properties are not evident from the sequence alone.

### Example 63

The following C. pneumoniae protein (PID 4377107) was expressed <SEQ ID 125; cp7107>:

```
    1 MSIVRNSALP LPCLSRSETF KKVRSHMKFM KVLTPWIYRK DLWVTAFLLT
  51 AIPGSFAHTL VDIAGEPRHA AQATGVSGDG KIVIGMKVPD DPFAITVGFQ
101 YIDGHLQPLE AVRPQCSVYP NGITPDGTVI VGTNYAIGMG SVAVKWVNGK
151 VSELPMLPDT LDSVASAVSA DGRVIGGNRN INLGASVAVK WEDDVITQLP
201 SLPDAMNACV NGISSDGSII VGTMVDVSWR NTAVQWIGDQ LSVIGTLGGT
251 TSVASAISTD GTVIVGGSEN ADSQTHAYAY KNGVMSDTGT LGGFYSLAHA
301 VSSDGSVIVG VSTNSEHRYH AFQYADGQMV DLGTLGGPES YAQGVSGDGK
351 VIVGRAQVPS GDWHAFLCPF QAPSPAPVHG GSTVVTSQNP RGMVDINATY
401 SSLKNSQQQL QRLLIQHSAK VESVSSGAPS FTSVKGAISK QSPAVQNDVQ
451 KGTFLSYRSQ VHGNVQNQQL LTGAFMDWKL ASAPKCGFKV ALHYGSQDAL
501 VERAALPYTE QGLGSSVLSG FGGQVQGRYD FNLGETVVLQ PFMGIQVLHL
551 SREGYSEKNV RFPVSYDSVA YSAATSFMGA HVFASLSPKM STAATLGVER
601 DLNSHIDEFK GSVSAMGNFV LENSTVSVLR PFASLAMYYD VRQQQLVTLS
651 VVMNQQPLTG TLSLVSQSSY NLSF*
```

The cp7107 nucleotide sequence <SEQ ID 126> is:

```
      1 ATGAGTATAG TCAGAAATTC TGCATTGCCA CTTCCGTGTT TAAGCAGATC
    51 CGAAACCTTT AAAAAAGTTA GGTCGCATAT GAAATTTATG AAAGTCCTTA
  101 CTCCATGGAT TTATCGAAAA GATCTTTGGG TAACAGCATT CTTACTGACA
  151 GCAATTCCAG GATCTTTTGC ACATACTCTT GTTGATATAG CAGGAGAACC
  201 TCGGCATGCT GCTCAAGCAA CAGGAGTTTC TGGAGATGGT AAAATTGTTA
  251 TAGGAATGAA AGTTCCGGAT GATCCTTTTG CTATAACTGT AGGATTTCAA
  301 TATATTGATG GGCATTTGCA ACCCTTAGAG GCAGTACGTC CTCAATGCTC
  351 TGTATACCCT AATGGTATAA CCCCGGACGG AACGGTTATT GTGGGTACAA
  401 ACTATGCCAT CGGGATGGGT AGTGTTGCTG TGAAATGGGT AAATGGCAAG
  451 GTTTCTGAAC TTCCCATGCT CCCTGACACC CTCGATTCTG TAGCATCGGC
  501 AGTTTCTGCA GATGGAAGAG TGATTGGAGG GAATAGAAAT ATAAATCTTG
  551 GCGCTTCTGT TGCTGTGAAA TGGGAGGACG ACGTGATTAC ACAACTTCCT
  601 TCTCTTCCTG ATGCTATGAA TGCTTGTGTT AACGGAATTT CTTCAGATGG
  651 TTCTATAATT GTAGGAACCA TGGTAGACGT GTCATGGAGA AATACCGCAG
  701 TACAATGGAT CGGGGATCAG CTCTCTGTTA TTGGGACTTT AGGAGGAACT
  751 ACTTCTGTTG CTAGTGCAAT CTCAACAGAT GGCACTGTGA TTGTAGGAGG
  801 TTCTGAAAAT GCAGATTCTC AGACTCATGC CTATGCTTAT AAAAACGGTG
  851 TTATGAGCGA TATAGGGACC CTCGGAGGTT TTTATTCTTT AGCACATGCA
  901 GTATCTTCAG ATGGTTCTGT GATTGTAGGA GTATCCACGA ACTCTGAGCA
  951 TAGATATCAT GCATTCCAAT ATGCTGATGG ACAGATGGTA GATTTAGGAA
1001 CTTTAGGAGG GCCTGAATCT TATGCTCAAG GTGTGTCTGG AGATGGAAAG
1051 GTAATTGTGG GTAGAGCACA AGTACCATCT GGAGATTGGC ATGCGTTCCT
1101 ATGTCCTTTC CAAGCTCCGA GCCCTGCTCC TGTCCATGGG GGAAGCACTG
1151 TCGTAACTAG CCAGAATCCA CGTGGAATGG TAGATATCAA TGCTACGTAC
1201 TCCTCTTTGA AAAATAGCCA ACAACAACTA CAAAGATTGC TTATCCAGCA
1251 TAGTGCAAAA GTTGAAAGTG TATCCTCAGG AGCACCATCT TTTACAAGTG
1301 TGAAAGGTGC GATCTCAAAA CAGAGCCCTG CAGTGCAAAA TGATGTACAG
1351 AAAGGGACGT TTTTAAGTTA CCGTTCCCAA GTTCATGGAA ACGTGCAGAA
1401 TCAGCAATTG CTCACAGGAG CTTTTATGGA CTGGAAACTC GCTTCAGCTC
1451 CTAAATGCGG CTTTAAAGTA GCTCTCCACT ATGGCTCTCA AGATGCTCTC
1501 GTAGAACGTG CAGCTCTTCC TTACACAGAA CAAGGCTTAG GAAGCAGTGT
1551 CTTGTCAGGT TTTGGAGGAC AAGTTCAAGG ACGCTATGAC TTTAATTTAG
1601 GAGAAACTGT TGTTCTGCAA CCCTTTATGG GCATTCAAGT TCTCCACCTA
1651 AGTAGAGAAG GGTATTCTGA GAAGAATGTT CGATTTCCTG TAAGCTATGA
1701 TTCTGTAGCC TACTCAGCAG CTACTAGCTT TATGGGTGCG CATGTATTTG
1751 CCTCCCTAAG CCCTAAAATG AGTACAGCAG CAACTTTAGG TGTGGAGAGA
1801 GATCTGAATT CACATATAGA TGAATTTAAG GGATCCGTCT CTGCTATGGG
1851 AAACTTTGTC TTGGAAAATT CTACAGTGAG TGTTTTAAGA CCTTTTGCTT
1901 CTCTTGCTAT GTACTATGAC GTAAGACAAC AGCAACTCGT GACGTTGTCA
1951 GTAGTTATGA ATCAACAACC CTTAACAGGC ACACTAAGCT TAGTAAGCCA
2001 AAGTAGCTAT AATCTTAGCT TCTAA
```

The PSORT algorithm predicts an inner membrane location (0.100).

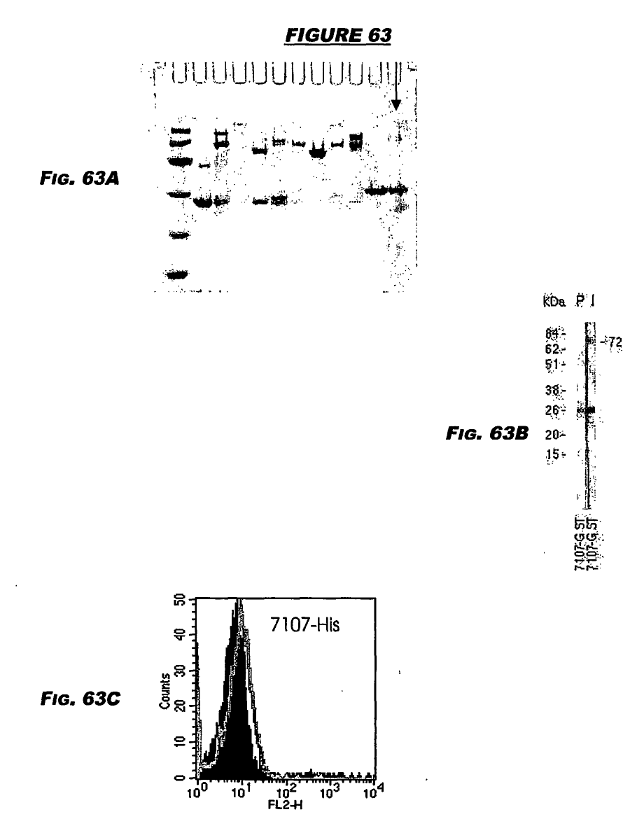

The protein was expressed in E. coli and purified as a GST-fusion (FIG. 63A) or his-tagged product. The proteins were used to immunize mice, whose sera were used in Western blot (FIG. 63B) and FACS (FIG. 63C) analyses.

These experiments show that cp7107 is a surface-exposed and immunoaccessible protein, and that it is a useful immunogen. These properties are not evident from the sequence alone.

### Example 64

The following C. pneumoniae protein (PID 4376467) was expressed <SEQ ID 127; cp6467>:

```
    1 MLRFFAVFIS TLWLITSGCS PSQSSKGIFV VNMKEMPRSL DPGKTRLIAD
  51 QTLMRHLYEG LVEEHSQNGE IKPALAESYT ISEDGTRYTF KIKNILWSNG
101 DPLTAQDFVS SWKEILKEDA SOVYLYAFLE IKNARAIFDD TESPENLGVR
151 ALDKRHLEIQ LETPCAHFLH FLTLPIFFPV HETLRNYSTS FEEMPITCGA
201 FRPVSLEKGL RLHLEKNPMY HNKSRVKLHK IIVQFISNAN TAAILFKHKK
251 LDWQGPPWGE PIPPEISASL HQDDQLFSLP GASTTWLLFN IQKKPWNNAK
301 LRKALSLAID KDMLTKVVYQ GLAEPTDHIL HPRLYPGTYP ERKRQNERTL
351 EAQQLFEEAL DELQMTREDL EKETLTFSTF SFSYGRICQM LREQWKKVLK
401 FTIPIVGQEF FTIQKNFLEG NYSLTVNQWT AAFIDPMSYL MIFANPGGIS
451 PYHLQDSHFQ TLLIKITQEH KKHLRNQLII EALDYLEHCH ILEPLCHPNL
501 RIALNKNIKN FNLFVRRTSD FRFIEKL*
```

A predicted signal peptide is highlighted.

The cp6467 nucleotide sequence <SEQ ID 128> is:

```
      1 ATGCTCCGTT TCTTCGCTGT ATTTATATCA ACTCTTTGGC TCATTACCTC
    51 AGGATGTTCC CCATCCCAAT CCTCTAAAGG AATTTTTGTG GTAAATATGA
  101 AGGAAATGCC ACGCTCCTTG GATCCTGGAA AAACTCGTCT CATTGCAGAC
  151 CAAACTCTAA TGCGTCATCT ATATGAAGGA CTCGTCGAAG AACATTCCCA
  201 AAATGGAGAG ATTAAACCAG CCCTTGCAGA AAGCTACACC ATCTCCGAAG
  251 ACGGGACTCG GTACACATTT AAAATCAAAA ACATCCTTTG GAGTAACGGA
  301 GACCCTCTGA CAGCTCAAGA CTTTGTCTCC TCTTGGAAGG AAATCCTAAA
  351 GGAAGATGCG TCCTCCGTAT ATCTCTATGC GTTTTTACCT ATCAAAAATG
  401 CTCGGGCAAT CTTTGATGAT ACTGAGTCTC CAGAAAATCT AGGAGTCCGA
  451 GCTTTAGATA AGCGTCATCT CGAAATTCAG TTAGAAACTC CCTGCGCGCA
  501 TTTCCTACAT TTCTTGACTC TTCCTATTTT TTTCCCTGTT CATGAAACTC
  551 TGCGAAACTA TAGCACCTCT TTTGAAGAGA TGCCCATTAC CTGCGGTGCT
  601 TTCCGCCCTG TGTCTCTAGA AAAAGGCCTG AGACTCCATC TAGAGAAAAA
  651 CCCTATGTAC CATAATAAAA GCCGTGTGAA ACTACATAAA ATTATTGTAC
  701 AGTTTATCTC AAACGCTAAC ACTGCAGCCA TTCTATTCAA ACATAAGAAA
  751 TTAGATTGGC AAGGACCTCC TTGGGGAGAA CCTATCCCTC CAGAAATCTC
  801 AGCTTCTCTA CATCAAGATG ACCAGCTCTT TTCTCTTCCG GGCGCTTCGA
  851 CTACATGGTT ACTCTTTAAT ATACAAAAAA AACCTTGGAA CAATGCTAAA
  901 TTACGCAAGG CATTGAGCCT TGCAATAGAC AAAGATATGT TAACCAAAGT
  951 GGTATACCAA GGTCTTGCAG AACCTACAGA TCATATCCTA CATCCAAGAC
1001 TTTATCCAGG GACCTATCCC GAACGGAAAA GACAAAACGA AAGAATTCTT
1051 GAGGCTCAAC AACTCTTTGA AGAAGCTCTA GACGAACTTC AAATGACACG
1101 CGAAGATCTA GAAAAGGAAA CTTTGACTTT CTCAACCTTT TCTTTTTCTT
1151 ACGGAAGGAT TTGCCAAATG CTAAGAGAAC AATGGAAGAA AGTCTTAAAA
1201 TTTACTATCC CTATAGTAGG CCAAGAGTTT TTCACAATAC AAAAAAACTT
1251 CCTAGAGGGG AACTATTCCC TAACCGTGAA CCAATGGACC GCAGCATTTA
1301 TTGATCCGAT GTCTTATCTC ATGATCTTTG CCAATCCTGG AGGAATTTCC
1351 CCCTATCACC TCCAAGATTC ACACTTTCAA ACTCTTCTCA TAAAGATCAC
1401 TCAAGAACAT AAAAAACACC TACGAAATCA GCTTATTATT GAAGCCCTTG
1451 ACTATTTAGA ACACTGTCAC ATTCTCGAAC CACTATGTCA TCCAAATCTT
1501 CGAATTGCTT TGAACAAAAA CATTAAAAAC TTTAATCTTT TTGTTCGACG
1551 AACTTCAGAC TTTCGTTTTA TAGAAAAACT ATAG
```

The PSORT algorithm predicts an outer membrane lipoprotein (0.790).

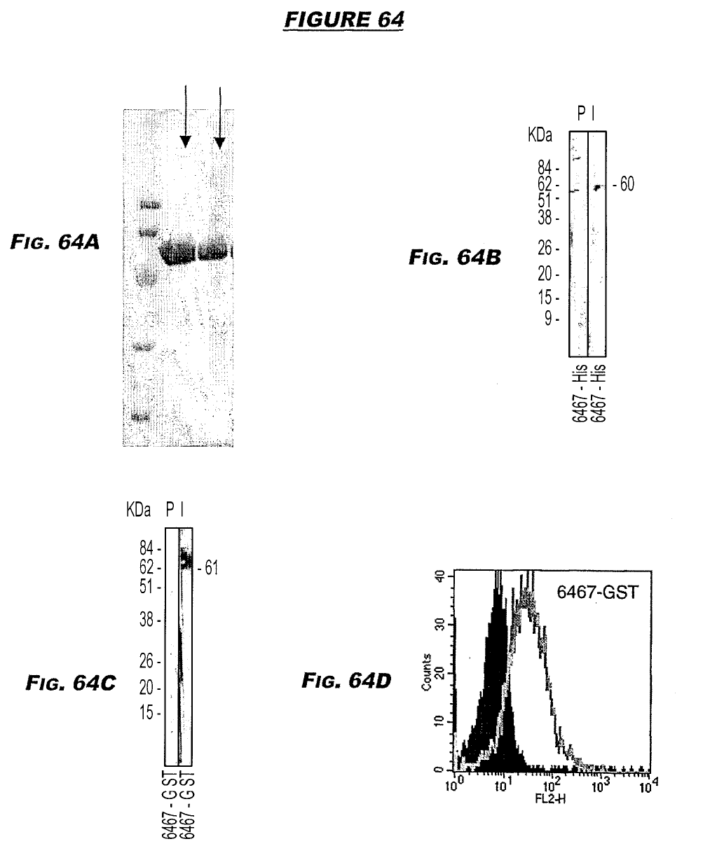

The protein was expressed in E. coli and purified as a his-tag product and a GST-fusion protein, as shown in FIG. 64A. The recombinant his-tag protein was used to immunize mice, whose sera were used in a Western blot (FIG. 64B). The recombinant GST-fusion protein was also used to immunize mice, whose sera were used in a Western blot (FIG. 64C) and for FACS analysis (FIG. 64D).

These experiments show that cp6467 is a surface-exposed and immunoaccessible protein, and that it is a useful immunogen. These properties are not evident from the sequence alone.

### Example 65

The following C. pneumoniae protein (PID 4376679) was expressed <SEQ ID 129; cp6679>:

```
    1 MRKMLVLLAS LGLLSPTLSS CTHLGSSGSY HPKLYTSGSK TKGVIAIYILPV
  51 FHRPGKSLEP LPWNLQGEFT EEISKRFYAS EKVFLIKHNA SPQTVSQFYA
101 PIANRLPETI IEQFLPAEFI VATELLEQKT GKEAGVDSVT ASVRVRVFDI
151 RHHKIALIYQ EIIECSQPLT TLVNDYHRYG WNSKHFDSTP MGLMHSRLFR
201 EVVARVEGYV CANYS*
```

A predicted signal peptide is highlighted.

The cp6679 nucleotide sequence <SEQ ID 130> is:

```
    1 ATGCGAAAAA TGTTGGTATT ATTGGCATCT TTAGGACTTC TATCCCCAAC
  51 CCTATCCAGC TGCACTCACT TAGGCTCTTC AGGAAGTTAT CATCCTAAGC
101 TATACACTTC AGGGAGCAAA ACTAAAGGTG TGATTGCGAT GCTTCCTGTA
151 TTTCATCGCC CAGGAAAGAG TCTTGAACCT TTACCTTGGA ACCTCCAAGG
201 AGAATTTACT GAAGAGATCA GCAAAAGGTT TTATGCTTCG GAAAAGGTCT
251 TCCTGATCAA GCACAATGCT TCACCTCAGA CAGTCTCTCA GTTCTATGCT
301 CCGATTGCGA ATCGTCTACC CGAAACAATT ATTGAGCAAT TTCTTCCTGC
351 AGAATTCATT GTTGCTACAG AACTGTTAGA ACAAAAGACA GGGAAAGAAG
401 CAGGTGTCGA TTCTGTAACA GCGTCTGTAC GTGTTCGCGT TTTTGATATC
451 CGTCATCATA AAATAGCTCT CATTTATCAA GAGATTATCG AATGCAGCCA
501 GCCTTTAACT ACCCTAGTCA ATGATTATCA TCGCTATGGC TGGAACTCAA
551 AACATTTTGA TTCAACGCCC ATGGGCTTAA TGCATAGCCG TCTTTTCCGC
601 GAAGTTGTTG CCAGAGTTGA GGGCTATGTT TGTGCTAACT ACTCGTAG
```

The PSORT algorithm predicts an inner membrane location (0.149).

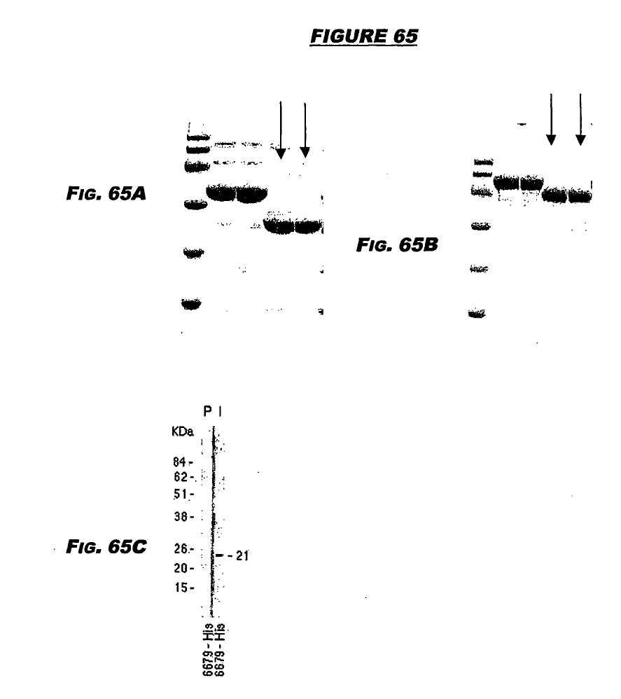

The protein was expressed in E. coli and purified as a his-tag product (FIG. 65A) and as a GST-fusion product (FIG. 65B). The recombinant protein was used to immunize mice, whose sera were used in a Western blot (FIG. 65C) and for FACS analysis.

These experiments show that cp6679 is a surface-exposed and immunoaccessible protein, and that it is a useful immunogen. These properties are not evident from the sequence alone.

### Example 66

The following C. pneumoniae protein (PID 4376890) was expressed <SEQ ID 131; cp6890>:

```
    1 MKQLLFCVCV FAMSCSAYAS PRRQDPSVMK ETFRNNYGII VSGQEWVKRG
  51 SDGTITKVLK NGATLHEVYS GGLLHGEITL TFPHTTALDV VQIYDQGRLV
101 SRKTFFVNGL ESQEELENED GTFVLTRWPD NNDSDTITKP YFIETTYQGH
151 VIEGSYTSFN GKYSSSIHNG EGVRSVFSSN NILLSEETFN EGVMVKYTTF
201 YPNRDPESIT HYQNGQPHGL RLTYLQGGIP NTIEEWRYGF QDGTTIVFKN
251 GCKTSEIAYV KGVKEGLELR YNEQEIVAEE VSWRNDFLHG ERKIYAGGTQ
301 KHEWYYRGRS VSKAKFERLN AAG*
```

A predicted signal peptide is highlighted.

The cp6890 nucleotide sequence <SEQ ID 132> is:

```
    1 ATGAAACAAT TACTTTTCTG TGTTTGCGTA TTTGCTATGT CATGTTCTGC
  51 TTACGCATCC CCACGACGAC AAGATCCTTC TGTTATGAAG GAAACATTCC
101 GAAATAATTA TGGCATTATT GTTTCCGGTC AAGAATGGGT AAAGCGTGGT
151 TCTGACGGCA CCATCACCAA AGTACTCAAA AATGGAGCTA CCCTGCATGA
201 AGTTTATTCT GGAGGCCTCC TTCATGGGGA AATTACCTTA ACGTTTCCCC
251 ATACCACAGC ATTGGACGTT GTTCAAATCT ATGATCAAGG TAGACTCGTT
301 TCTCGCAAAA CCTTTTTTGT GAACGGTCTT CCATCTCAAG AAGAGCTGTT
351 CAATGAAGAT GGCACGTTTG TCCTCACACG ATGGCCGGAC AACAACGACA
401 GTGATACCAT CACAAAGCCT TACTTCATAG AAACGACATA TCAAGGGCAT
451 GTCATAGAAG GAAGTTATAC TTCCTTTAAT GGGAAATACT CCTCATCCAT
501 CCACAATGGA GAGGGAGTTC GTTCTGTGTT CTCCTCCAAT AACATCCTTC
551 TTTCTGAAGA GACCTTCAAT GAAGGTGTCA TGGTGAAATA TACCACATTC
601 TATCCGAATC GCGATCCCGA ATCGATTACT CATTATCAAA ATGGACAGCC
651 TCACGGCTTA CGGCTAACAT ATCTACAAGG TGGCATCCCC AATACGATAG
701 AGGAGTGGCG TTATGGCTTT CAAGACGGAA CGACCATCGT ATTTAAAAAT
751 GGTTGTAAGA CATCTGAGAT CGCTTATGTT AAGGGAGTGA AAGAAGGTTT
801 AGAACTGCGC TACAATGAAC AGGAAATTGT AGCTGAAGAA GTTTCTTGGC
851 GTAATGATTT TCTGCATGGA GAACGTAAGA TCTATGCTGG AGGAATCCAA
901 AAGCATGAAT GGTATTACCG CGGGAGATCT GTATCTAAAG CCAAATTCGA
951 GCGGCTAAAT GCTGCAGGAT AG
```

The PSORT algorithm predicts an outer membrane location (0.940).

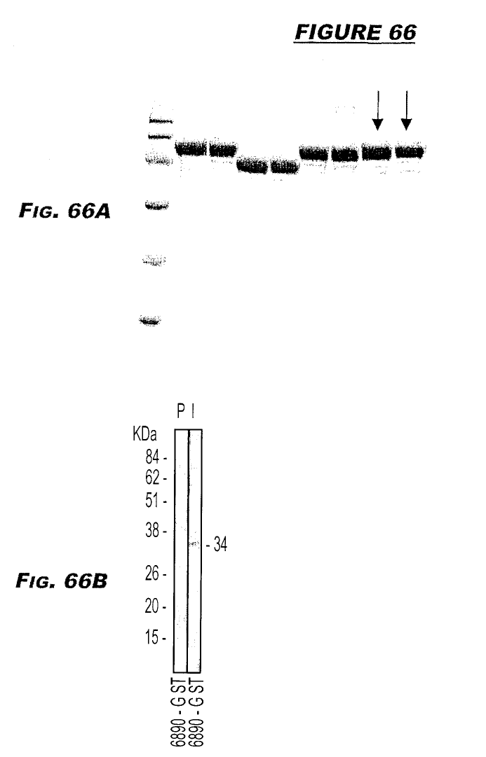

The protein was expressed in E. coli and purified as a GST-fusion product, as shown in FIG. 66A. The recombinant protein was used to immunize mice, whose sera were used in a Western blot (FIG. 66B) and for FACS analysis. A his-tagged protein was also expressed.

These experiments show that cp6890 is a surface-exposed and immunoaccessible protein, and that it is a useful immunogen. These properties are not evident from the sequence alone.

### Example 67

The following C. pneumoniae protein (PID 6172323) was expressed <SEQ ID 133; cp0018>:

```
    1 MKTSVSMLLA LLCSGASSIV LHAATTPLNP EDGFIGEGNT NTFSPKSTTD
  51 AAGTTYSLTG EVLYIDPGKG GSITGTCFVE TAGDLTFLGN GNTLKFLSVD
101 AGANIAVAHV QGSKNLSFTD FLSLVITESP KSAVTTGKGS LVSLGAVQLQ
151 DINTLVLTSN ASVEDGGVIK GNSCLIQGIK NSAIFGQNTS SKKGGAISTT
201 QGLTIENNLG TLKFNENKAV TSGGALDLGA ASTFTANHEL IFSQNKTSGN
251 AANGGAINCS GDLTFTDNTS LLLQENSTMQ DGGALCSTGT ISITGSDSIN
301 VIGNTSGQKG GAISAASLKI LGGQGGALFS NNVVTHATPL GGAIFINTGG
351 SLQLFTQGGD IVFEGNQVTT TAPNATTKRN VIHLESTAKW TGLAASQGNA
401 IYFYDPITTN DTGASDNLRI NEVSANQKLS GSIVFSGERL STAEAIAENL
451 TSRINQPVTL VEGSLVLKQG VTLITQGFSQ EPESTLLLDL GTSL*
```

A predicted signal peptide is highlighted.

The cp0018 nucleotide sequence <SEQ ID 134> is:

```
      1 ATGAAGACTT CAGTTTCTAT GTTGTTGGCC CTGCTTTGCT CGGGGGCTAG
    51 CTCTATTGTA CTCCATGCCG CAACCACTCC ACTAAATCCT GAAGATGGGT
  101 TTATTGGGGA GGGCAATACA AATACTTTTT CTCCGAAATC TACAACGGAT
  151 GCTGCAGGAA CTACCTACTC TCTCACAGGA GAGGTTCTGT ATATAGATCC
  201 GGGGAAAGGT GGTTCAATTA CAGGAACTTG CTTTGTAGAA ACTGCTGGCG
  251 ATCTTACATT TTTAGGTAAT GGAAATACCC TAAAGTTCCT GTCGGTAGAT
  301 GCAGGTGCTA ATATCGCGGT TGCTCATGTA CAAGGAAGTA AGAATTTAAG
  351 CTTCACAGAT TTCCTTTCTC TGGTGATCAC AGAATCTCCA AAATCCGCTG
  401 TTACTACAGG AAAAGGTAGC CTAGTCAGTT TAGGTGCAGT CCAACTGCAA
  451 GATATAAACA CTCTAGTTCT TACAAGCAAT GCCTCTGTCG AAGATGGTGG
  501 CGTGATTAAA GGAAACTCCT GCTTGATTCA GGGAATCAAA AATAGTGCGA
  551 TTTTTGGACA AAATACATCT TCGAAAAAAG GAGGGGCGAT CTCCACGACT
  601 CAAGGACTTA CCATAGAGAA TAACTTAGGG ACGCTAAAGT TCAATGAAAA
  651 CAAAGCAGTG ACCTCAGGAG GCGCCTTAGA TTTAGGAGCC GCGTCTACAT
  701 TCACTGCGAA CCATGAGTTG ATATTTTCAC AAAATAAGAC TTCTGGGAAT
  751 GCTGCAAATG GCGGAGCCAT AAATTGCTCA GGGGACCTTA CATTTACTGA
  801 TAACACTTCT TTGTTACTTC AAGAAAATAG CACAATGCAG GATGGTGGAG
  851 CTTTGTGTAG CACAGGAACC ATAAGCATTA CCGGTAGTGA TTCTATCAAT
  901 GTGATAGGAA ATACTTCAGG ACAAAAAGGA GGAGCGATTT CTGCAGCTTC
  951 TCTCAAGATT TTGGGAGGGC AGGGAGGCGC TCTCTTTTCT AATAACGTAG
1001 TGACTCATGC CACCCCTCTA GGAGGTGCCA TTTTTATCAA CACAGGAGGA
1051 TCCTTGCAGC TCTTCACTCA AGGAGGGGAT ATCGTATTCG AGGGGAATCA
1101 GGTCACTACA ACAGCTCCAA ATGCTACCAC TAAGAGAAAT GTAATTCACC
1151 TCGAGAGCAC CGCGAAGTGG ACGGGACTTG CTGCAAGTCA AGGTAACGCT
1201 ATCTATTTCT ATGATCCCAT TACCACCAAC GATACGGGAG CAAGCGATAA
1251 CTTACGTATC AATGAGGTCA GTGCAAATCA AAAGCTCTCG GGATCTATAG
1301 TATTTTCTGG AGAGAGATTG TCGACAGCAG AAGCTATAGC TGAAAATCTT
1351 ACTTCGAGGA TCAACCAGCC TGTCACTTTA GTAGAGGGGA GCTTAGTACT
1401 TAAACAGGGA GTGACCTTGA TCACACAAGG ATTCTCGCAG GAGCCAGAAT
1451 CCACGCTTCT TTTGGATCTG GGGACCTCAT TATAA
```

The PSORT algorithm predicts outer membrane (0.935).

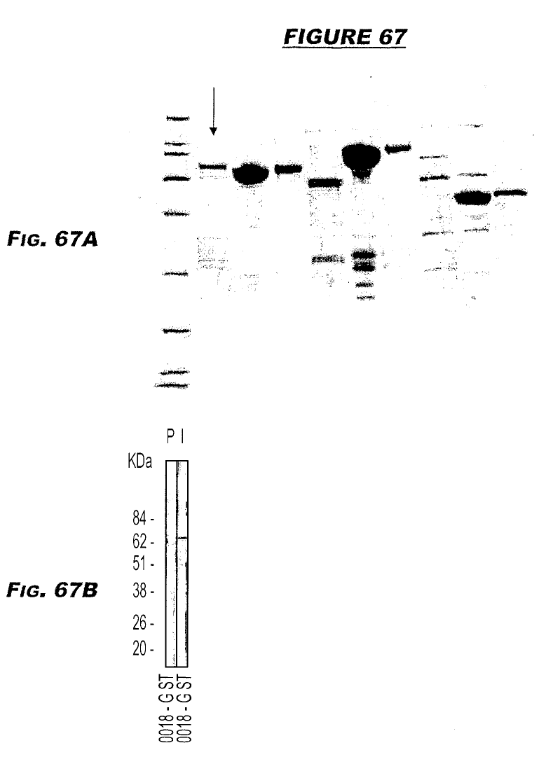

The protein was expressed in E. coli and purified as a GST-fusion product (FIG. 67A). The recombinant protein was used to immunize mice, whose sera were used in a Western blot (FIG. 67B) and for FACS analysis.

These experiments show that cp0018 is a surface-exposed and immunoaccessible protein, and that it is a useful immunogen. These properties are not evident from the sequence alone.

### Example 68

The following C. pneumoniae protein (PID 4376262) was expressed <SEQ ID 135; cp6262>:

```
    1 MRKLRILAIV LIALSIILIA GGVVLLTVAI PGLSSVISSP AGMGACALGC
  51 VMLALGIDVL LKKREVPIVL ASVTTTPGTG SPRSGISISG ADSTIRSLPT
101 YLLDEGHPQS MRKLRTLATV LIVFSIILIA SGVVLLTVAI PGLSSVISSP
151 AGMGACALGC VMLALGIDVL LKKREVPIVL ASVTTTPGTG SPRSGISISG
201 ADSTIRSLPT YPLDEGHPQS MRKLRILAIV LIVESIILIA SGVVLLTVAI
251 PGLSSIISSP AEMGACALGC VMLALGIDVL LKKREVPIVV PAPIPEEVVI
301 DDIDEESIRL QQEAEAALAR LPEEMSAFEG YTKVVESHLE NMKSLPYDGH
351 GLEEKTKHQI RVVRSSLKAM VPEFLDIRRI FEEEEFFFLS ARKRLIDLAT
401 TLVERKILTE QLERNNLRKA FSYLYQDSIF KKIIDNFEKL AWKFMILSKS
451 ICRETIIFEN HEHGVAKSLL HKNAVLLEKV IYRSLQKSYR DIGMSSAKMK
501 ILHGNPFFSL EDNKKTIMKE HAEMLESLSS YRKVFLALSD ENVVDTPSDP
551 KKWDLSGIPC RDALSEISRD EQWQKKAHLK HQESLYTQAR DRLTDQSSKE
601 NQKELEKAEQ EYISSWERVK KFEIERVQER IRAIQKLYPN ILEREEETTG
651 QETVTPTVQG TTASSDLTDI LGRIEVSSRE DNQNQESCVK VLRSHEVEMS
701 WEVKQEYGPK KKEFQDQMGS LERFETEHIE ELEVLQKDYS KHLSYFKKVN
751 NKKEVQYAKF RLKVLESDLE GILAQTESAE SLLTQEELPI LATRGALEKA
801 VFKGSLCCAL ASKAKEYFEE DPRFQDSDTQ LRALTLRLQE AKASLEEEIK
851 RFSNLENDIA EERRLLKESK QTFERAGLGV LREIAVESTY DLRSLTNTWE
901 GTPESEKVYF SMYLNYYNEE KRRAKTRLVE MTQRYRDFKM ALEAMQFNEE
951 ALLQEELSIQ APSE*
```

A predicted signal peptide is highlighted.

The cp6262 nucleotide sequence <SEQ ID 136> is:

```
      1 ATGAGGAAAC TTCGTATTCT TGCGATCGTT CTCATAGCTT TGAGCATTAT
    51 TTTGATTGCA GGTGGTGTGG TATTGCTTAC TGTAGCGATC CCTGGATTAA
  101 GTTCAGTCAT TTCTTCCCCG GCAGGGATGG GTGCCTGTGC TTTGGGATGT
  151 GTGATGCTTG CTTTAGGGAT CGATGTTCTT CTGAAGAAAC GAGAAGTCCC
  201 TATAGTTCTC GCATCTGTAA CTACGACACC AGGAACTGGC AGCCCTAGAA
  251 GTGGTATTTC TATTTCAGGA GCTGATAGCA CCATACGTTC TCTTCCTACG
  301 TATCTCTTGG ACGAGGGACA TCCACAATCC ATGAGGAAAC TTCGTATTCT
  351 TGCGATCGTT CTCATAGTTT TTAGCATTAT TTTGATTGCA AGTGGTGTGG
  401 TATTGCTTAC TGTAGCGATC CCTGGATTAA GTTCAGTCAT TTCTTCCCCG
  451 GCAGGGATGG GTGCCTGTGC TTTGGGATGT GTGATGCTTG CTTTAGGGAT
  501 CGATGTTCTT CTGAAGAAAC GAGAAGTCCC TATAGTTCTC GCATCTGTAA
  551 CTACGACACC AGGAACTGGC AGCCCTAGAA GTGGTATTTC TATTTCAGGA
  601 GCTGATAGCA CCATACGTTC TCTTCCTACG TATCCCTTGG ACGAGGGACA
  651 TCCACAATCC ATGAGGAAAC TTCGTATTCT TGCGATCGTT CTCATAGTTT
  701 TTAGCATTAT TTTGATTGCA AGTGGTGTGG TATTGCTTAC TGTAGCGATC
  751 CCTGGATTAA GCTCGATCAT TTCTTCCCCA GCGGAGATGG GTGCTTGTGC
  801 TTTGGGATGT GTGATGCTTG CTTTGGGGAT CGACGTTCTT CTGAAGAAAC
  851 GAGAAGTCCC TATAGTAGTT CCCGCACCTA TTCCTGAAGA AGTCGTCATA
  901 GATGATATAG ATGAAGAGAG TATACGGCTG CAGCAGGAAG CTGAAGCCGC
  951 TTTAGCAAGA CTTCCTGAGG AGATGAGTGC ATTTGAAGGT TACATAAAAG
1001 TTGTCGAGAG TCATTTGGAG AACATGAAAA GCCTGCCTTA TGATGGTCAT
1051 GGGCTAGAAG AGAAAACGAA ACATCAGATA AGAGTCGTCA GATCTTCTTT
1101 GAAGGCTATG GTTCCAGAAT TTTTAGATAT CAGAAGAATT TTTGAAGAAG
1151 AAGAGTTCTT TTTTCTCTCA GCTCGCAAAC GACTTATAGA TTTAGCTACT
1201 ACTTTAGTAG AGAGAAAAAT TTTAACAGAG CAACTTGAGC GCAATAATTT
1251 AAGGAAAGCG TTTTCTTATT TATATCAGGA CTCAATTTTT AAAAAAATTA
1301 TTGATAACTT CGAGAAGTTA GCATGGAAAT TTATGATTTT GAGTAAATCA
1351 ATTTGTCGAT TTACAATTAT TTTTGAAAAT CATGAACATG GTGTAGCAAA
1401 GAGCCTGTTA CACAAGAATG CAGTGTTACT GGAGAAGGTA ATCTATAGGA
1451 GTTTGCAAAA AAGCTATAGA GATATAGGCA TGTCATCTGC AAAGATGAAA
1501 ATCTTGCACG GCAACCCTTT TTTCTCTTTG GAAGATAATA AAAAGACGAT
1551 AATGAAAGAA CACGCAGAGA TGCTTGAAAG TCTCAGTAGC TATAGGAAGG
1601 TATTTTTAGC TCTATCTGAT GAGAACGTTG TAGATACACC TAGCGATCCA
1651 AAGAAATGGG ATTTGTCAGG AATCCCCTGT AGGGACGCGT TGTCTGAGAT
1701 TTCTCGTGAT GAACAGTGGC AGAAGAAAGC ACATCTAAAG CATCAAGAGT
1751 CCCTCTATAC GCAAGCTAGG GATCGTTTAA CAGACCAGAG CTCTAAAGAA
1801 AATCAGAAAG AGTTAGAGAA AGCTGAACAA GAGTACATAT CTTCTTGGGA
1851 ACGGGTTAAA AAATTTGAGA TTGAGAGAGT ACAGGAGAGG ATACGGGCAA
1901 TTCAAAAGCT TTATCCTAAT ATCCTCGAGA GAGAAGAAGA AACCACAGGT
1951 CAGGAGACTG TGACTCCAAC TGTTCAAGGG ACGACGGCTT CATCCGATTT
2001 AACAGATATT TTAGGAAGAA TAGAGGTCTC CAGTAGGGAG GATAATCAGA
2051 ATCAAGAGTC TTGTGTAAAA GTCTTAAGAA GTCATGAGGT AGAAATGAGC
2101 TGGGAAGTCA AACAAGAGTA TGGCCCTAAG AAAAAAGAAT TTCAGGATCA
2151 AATGGGTTCT TTAGAGAGGT TTTTTACAGA GCATATTGAA GAGTTAGAAG
2201 TATTACAGAA GGACTACTCT AAACACTTGT CTTATTTTAA AAAAGTAAAC
2251 AATAAGAAAG AGGTTCAATA TGCGAAGTTT AGGTTGAAGG TTTTAGAGTC
2301 AGATTTAGAA GGGATTCTAG CTCAGACTGA GAGTGCTGAG AGTCTGTTAA
2351 CTCAAGAAGA ACTTCCGATT CTTGCAACTC GGGGAGCCTT AGAGAAAGCT
2401 GTTTTCAAAG GGAGTCTATG TTGCGCGCTA GCAAGCAAAG CAAAACCCTA
2451 TTTTGAAGAG GATCCCAGAT TCCAAGATTC TGATACGCAA TTGCGAGCTC
2501 TGACTCTAAG GTTACAGGAG GCTAAGGCAA GCCTGGAAGA AGAGATAAAG
2551 AGATTTTCAA ATCTTGAGAA CGATATTGCA GAGGAAAGAC GCCTTCTTAA
2601 AGAGAGCAAG CAGACGTTCG AAAGAGCAGG TTTAGGGGTT CTCCGAGAAA
2651 TTGCAGTCGA GTCTACTTAT GATTTGCGTT CCTTAACAAA TACATGGGAA
2701 GGGACCCCAG AGAGTGAGAA GGTCTATTTT AGCATGTATC TTAATTATTA
2751 CAACGAAGAG AAACGTAGGG CTAAAACAAG ATTGGTTGAA ATGACACAGA
2801 GGTATAGAGA TTTTAAAATG GCCTTGGAAG CTATGCAGTT TAATGAAGAA
2851 GCCCTTTTGC AAGAGGAACT CTCTATTCAA GCTCCCAGTG AATAA
```

The PSORT algorithm predicts inner membrane (0.660).

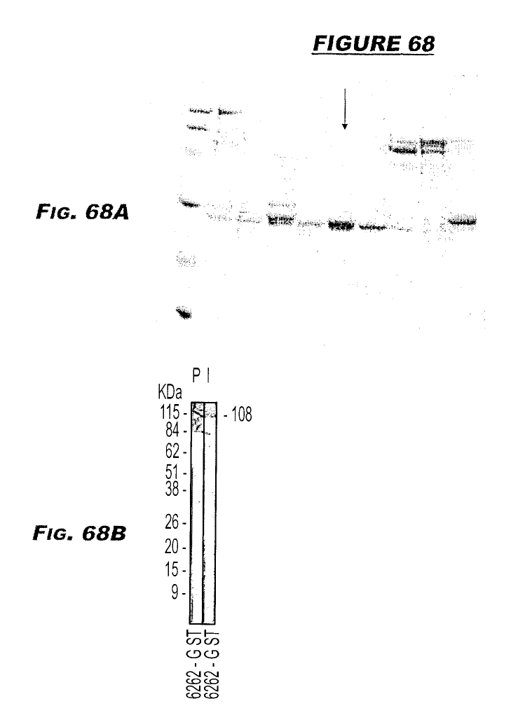

The protein was expressed in E. coli and purified as a GST-fusion product (FIG. 68A). The recombinant protein was used to immunize mice, whose sera were used in a Western blot (FIG. 68B) and for FACS analysis.

These experiments show that cp6262 is a surface-exposed and immunoaccessible protein, and that it is a useful immunogen. These properties are not evident from the sequence alone.

### Example 69

The following C. pneumoniae protein (PID 4376269) was expressed <SEQ ID 137; cp6269>:

```
    1 MYQENLRLLE RLLYNSVQKS YADRLFSYEK TKMVHDTPLI PWEEDKEKCA
  51 EAEKAFLEQQ KILLDYGKSI FWLNENDEIN LNDPWSWGLN TVRTRKVFQE
101 VDDSERWNHK VLIQKLEDDY EKLLEESSKE STEANKKLLS DLVDRLEDAK
151 TKFFLKKQEE VETRVKDLRA RYGGTVDPKQ DTEAKKKVEL EASLETFLDS
201 IESELVQCLE DQDIYWKEQD VKDLARTQEL EEQDIEAKRE EAAEDLRSLN
251 ERLKKSKTML DRAKWHIENA EDSITWWTSQ IEMKDMKARL KILKEDITSV
301 LPEIDEIETC LSLEELPLLT TRELLTKSYL KFKICSETLL KMTSVFENNI
351 YVQEYEVQLQ NLGFKLQGTS QRFGKKQDDF ANLEEQVALQ KKRLRELTQN
401 FEIQGFNFMK EDFKAAAKDL YIRSTAEQKM NFDVPCMELF RRYHEEVNKP
451 LLELMYNCAD SYRDAKKKLC SLRLDEKELL QKEIKKEEFY QKKQQRHADR
501 SRHTTYQKLR IAEELALELK KKI*
```

The cp6269 nucleotide sequence <SEQ ID 138> is:

```
      1 ATGTACCAGG AGAATCTAAG ATTGTTGGAA AGGCTTCTTT ATAATAGTGT
    51 TCAAAAGAGC TATGCGGATC GGCTGTTTTC CTATGAAAAG ACAAAGATGG
  101 TGCACGATAC TCCGCTGATT CCTTGGGAAG AGGATAAGGA AAAATGTGCT
  151 GAAGCTGAGA AAGCTTTCTT AGAGCAACAG AAGATTCTCC TAGATTATGG
  201 AAAATCTATC TTTTGGCTGA ATGAGAACGA TGAGATCAAT TTAAACGATC
  251 CTTGGAGTTG GGGTCTTAAT ACGGTGAGGA CTAGGAAAGT ATTCCAAGAG
  301 GTTGACGACA GTGAACGTTG GAATCATAAG GTACTCATTC AAAAACTCGA
  351 GGACGATTAT GAGAAACTTC TAGAGGAAAG TTCAAAAGAG TCTACTGAAG
  401 CAAATAAGAA GCTTTTATCT GACTTAGTAG ATCGTCTTGA AGATGCTAAG
  451 ACAAAATTTT TCCTGAAGAA ACAGGAGGAG GTGGAGACTC GCGTTAAGGA
  501 TCTTAGAGCT CGATATGGAG GCACAGTAGA TCCTAAGCAG GATACGGAAG
  551 CTAAGAAGAA AGTCGAATTG GAGGCTAGCT TAGAAACCTT TTTAGATTCC
  601 ATCGAATCAG AGCTAGTACA GTGTTTAGAA GATCAAGATA TATATTGGAA
  651 AGAACAGGAT GTCAAAGATC TAGCACGTAC GCAAGAGCTC GAGGAACAAG
  701 ATATTGAAGC GAAGAGGGAA GAAGCTGCCG AAGACCTAAG AAGTCTTAAT
  751 GAGCGTTTAA AGAAGTCAAA AACTATGTTA GATAGGGCTA AATGGCATAT
  801 TGAAAATGCT GAGGACAGTA TTACCTGGTG GACTAGTCAG ATAGAAATGA
  851 AGGATATGAA AGCAAGACTG AAGATCTTAA AAGAAGATAT AACAAGTGTT
  901 CTACCTGAAA TAGATGAGAT TGAAACGTGT TTAAGCTTAG AGGAGCTTCC
  951 TTTGCTTACG ACCAGGGAAC TCTTAACTAA GTCCTACCTA AAGTTTAAGA
1001 TTTGTTCGGA AACACTATTA AAAATGACTT CTGTGTTTGA GAACAATATC
1051 TATGTTCAGG AGTACGAGGT TCAGCTGCAA AATCTAGGGT TTAAGTTACA
1101 AGGTATATCT CAGAGATTCG GAAAGAAACA AGACGATTTT GCGAATCTAG
1151 AGGAACAGGT TGCTTTGCAA AAGAAACGAC TCAGAGAGCT CACTCAGAAT
1201 TTTGAAATAC AAGGATTCAA TTTCATGAAA GAAGATTTTA AGGCAGCCGC
1251 TAAAGATCTT TATATAAGAA GTACAGCTGA ACAAAAGATG AACTTTGATG
1301 TGCCTTGCAT GGAGCTCTTC CGTAGGTATC ATGAGGAGGT CAACAAGCCG
1351 CTTCTTGAGT TGATGTACAA TTGTGCAGAC AGTTATAGAG ATGCTAAGAA
1401 AAAGCTTTGC TCTCTACGTC TTGATGAAAA AGAGTTATTA CAAAAAGAAA
1451 TCAAGAAAGA GGAATTTTAT CAAAAGAAAC AACAAAGGCA TGCAGATAGA
1501 TCACGTCATA CTACGTATCA AAAGCTACGA ATTGCTGAAG AGCTTGCTCT
1551 TGAGCTGAAG AAGAAAATCT AA
```

The PSORT algorithm predicts cytoplasmic location (0.412).

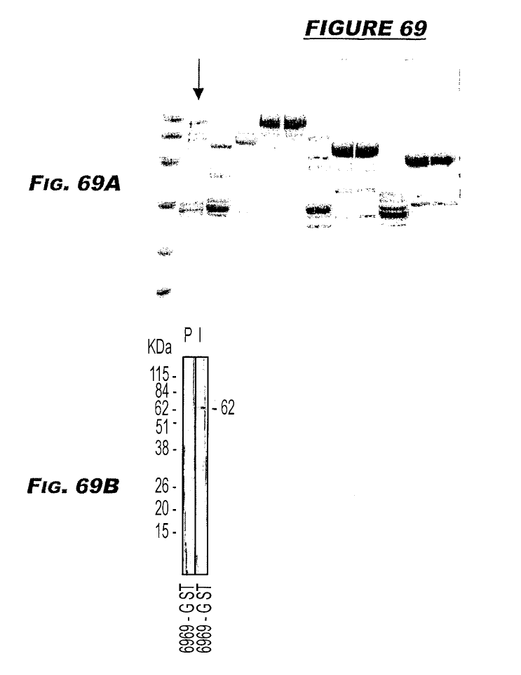

The protein was expressed in E. coli and purified as a GST-fusion product (FIG. 69A). The recombinant protein was used to immunize mice, whose sera were used in a Western blot (FIG. 69B) and for FACS analysis.

These experiments show that cp6269 is a surface-exposed and immunoaccessible protein, and that it is a useful immunogen. These properties are not evident from the sequence alone.

### Example 70

The following C. pneumoniae protein (PID 4376270) was expressed <SEQ ID 139; cp6270>:

```
    1 MKIPLRFLLI SLVPTLSMSN LLGAATTEEL SASNSFDGTT STTSFSSKTS
  51 SATDGTNYVF KDSVVIENVP KTGETQSTSC FKNDAAAGDL NFLGGGFSFT
101 FSNIDATTAS GAAIGSEAAN KTVTLSGFSA LSFLKSPAST VTNGLGAINV
151 KGNLSLLDND KVLIQDNFST GDGGAINCAG SLKIANNKSL SFIGNSSSTR
201 GGAIHTKNLT LSSGGETLFQ GNTAPTAAGK GGAIAIADSG TLSISGDSGD
251 IIFEGNTIGA TGTVSHSAID LGTSAKITAL RAAQGHTIYF YDPITVTGST
301 SVADALNINS PDTGDNKEYT GTIVFSGEKL TEAEAKDEKN RTSKLLQNVA
351 FKNGTVVLKG DVVLSANGFS QDANSKLIMD LGTSLVANTE SIELTNLEIN
401 IDSLRNGKKI KLSAATAQKD IRIDRPVVLA ISDESFYQNG FLNEDHSYDG
451 ILELDAGKDI VISADSRSID AVQSPYGYQG KWTINWSTDD KKATVSWAKQ
501 SFNPTAEQEA PLVPNLLWGS FIDVRSFQNF IELGTEGAPY EKRFWVAGIS
551 NVLHRSGREN QRKFRHVSGG AVVGASTRMP GGDTLSLGFA QLFARDKDYF
501 MNTNFAKTYA GSLRLQHDAS LYSVVSILLG EGGLREILLP YVSKTLPCSF
651 YGQLSYGHTD HRMKTESLPP PPPTLSTDHT SWGGYVWAGE LGTRVAVENT
701 SGRGFFQEYT PFVKVQAVYA RQDSFVELGA ISRDFSDSHL YNLAIPLGTK
751 LEKRFAEQYY HVVAMYSPDV CRSNPKCTTT LLSNQGSWKT KGSNLARQAG
801 IVQASGFRSL GAAAELFGNF GFEWRGSSRS YNVDAGSKIK F*
```

A predicted signal peptide is highlighted.

The cp6270 nucleotide sequence <SEQ ID 140> is:

```
      1 ATGAAGATTC CACTCCGCTT TTTATTGATA TCATTAGTAC CTACGCTTTC
    51 TATGTCGAAT TTATTAGGAG CTGCTACTAC CGAAGAGTTA TCGGCTAGCA
  101 ATAGCTTCGA TGGAACTACA TCAACAACAA GCTTTTCTAG TAAAACATCA
  151 TCGGCTACAG ATGGCACCAA TTATGTTTTT AAAGATTCTG TAGTTATAGA
  201 AAATGTACCC AAAACAGGGG AAACTCAGTC TACTAGTTGT TTTAAAAATG
  251 ACGCTGCAGC TGGAGATCTA AATTTCTTAG GAGGGGGATT TTCTTTCACA
  301 TTTAGCAATA TCGATGCAAC CACGGCTTCT GGAGCTGCTA TTGGAAGTGA
  351 AGCAGCTAAT AAGACAGTCA CGTTATCAGG ATTTTCGGCA CTTTCTTTTC
  401 TTAAATCCCC AGCAAGTACA GTGACTAATG GATTGGGAGC TATCAATGTT
  451 AAAGGGAATT TAAGCCTATT GGATAATGAT AAGGTATTGA TTCAGGACAA
  501 TTTCTCAACA GGAGATGGCG GAGCAATTAA TTGTGCAGGC TCCTTGAAGA
  551 TCGCAAACAA TAAGTCCCTT TCTTTTATTG GAAATAGTTC TTCAACACGT
  601 GGCGGAGCGA TTCATACCAA AAACCTCACA CTATCTTCTG GTGGGGAAAC
  651 TCTATTTCAG GGGAATACAG CGCCTACGGC TGCTGGTAAA GGAGGTGCTA
  701 TCGCGATTGC AGACTCTGGC ACCCTATCCA TTTCTGGAGA CAGTGGCGAC
  751 ATTATCTTTG AAGGCAATAC GATAGGAGCT ACAGGAACCG TCTCTCATAG
  801 TGCTATTGAT TTAGGAACTA GCGCTAAGAT AACTGCGTTA CGTGCTGCGC
  851 AAGGACATAC GATATACTTT TATGATCCGA TTACTGTAAC AGGATCGACA
  901 TCTGTTGCTG ATGCTCTCAA TATTAATAGC CCTGATACTG GAGATAACAA
  951 AGAGTATACG GGAACCATAG TCTTTTCTGG AGAGAAGCTC ACGGAGGCAG
1001 AAGCTAAAGA TGAGAAGAAC CGCACTTCTA AATTACTTCA AAATGTTGCT
1051 TTTAAAAATG GGACTGTAGT TTTAAAAGGT GATGTCGTTT TAAGTGCGAA
1101 CGGTTTCTCT CAGGATGCAA ACTCTAAGTT GATTATGGAT TTAGGGACGT
1151 CGTTGGTTGC AAACACCGAA AGTATCGAGT TAACGAATTT GGAAATTAAT
1201 ATAGACTCTC TCAGGAACGG GAAAAAGATA AAACTCAGTG CTGCCACAGC
1251 TCAGAAAGAT ATTCGTATAG ATCGTCCTGT TGTACTGGCA ATTAGCGATG
1301 AGAGTTTTTA TCAAAATGGC TTTTTGAATG AGGACCATTC CTATGATGGG
1351 ATTCTTGAGT TAGATGCTGG GAAAGACATC GTGATTTCTG CAGATTCTCG
1401 CAGTATAGAT GCTGTACAAT CTCCGTATGG CTATCAGGGA AAGTGGACGA
1451 TCAATTGGTC TACTGATGAT AAGAAAGCTA CGGTTTCTTG GGCGAAGCAG
1501 AGTTTTAATC CCACTGCTGA GCAGGAGGCT CCGTTAGTTC CTAATCTTCT
1551 TTGGGGTTCT TTTATAGATG TTCGTTCCTT CCAGAATTTT ATAGAGCTAG
1601 GTACTGAAGG TGCTCCTTAC GAAAAGAGAT TTTGGGTTGC AGGCATTTCC
1651 AATGTTTTGC ATAGGAGCGG TCGTGAAAAT CAAAGGAAAT TCCGTCATGT
1701 GAGTGGAGGT GCTGTAGTAG GTGCTAGCAC GAGGATGCCG GGTGGTGATA
1751 CCTTGTCTCT GGGTTTTGCT CAGCTCTTTG CGCGTGACAA AGACTACTTT
1801 ATGAATACCA ATTTCGCAAA GACCTACGCA GGATCTTTAC GTTTGCAGCA
1851 CGATGCTTCC CTATACTCTG TGGTGAGTAT CCTTTTAGGA GAGGGAGGAC
1901 TCCGCGAGAT CCTGTTGCCT TATGTTTCCA AGACTCTGCC GTGCTCTTTC
1951 TATGGGCAGC TTAGCTACGG CCATACGGAT CATCGCATGA AGACCGAGTC
2001 TCTACCCCCC CCCCCCCCGA CGCTCTCGAC GGATCATACT TCTTGGGGAG
2051 GATATGTCTG GGCTGGAGAG CTGGGAACTC GAGTTGCTGT TGAAAATACC
2101 AGCGGCAGAG GATTTTTCCA AGAGTACACT CCATTTGTAA AAGTCCAAGC
2151 TGTTTACGCT CGCCAAGATA GCTTTGTAGA ACTAGGAGCT ATCAGTCGTG
2201 ATTTTAGTGA TTCGCATCTT TATAACCTTG CGATTCCTCT TGGAATCAAG
2251 TTAGAGAAAC GGTTTGCAGA GCAATATTAT CATGTTGTAG CGATGTATTC
2301 TCCAGATGTT TGTCGTAGTA ACCCCAAATG TACGACTACC CTACTTTCCA
2351 ACCAAGGGAG TTGGAAGACC AAAGGTTCGA ACTTAGCAAG ACAGGCTGGT
2401 ATTGTTCAGG CCTCAGGTTT TCGATCTTTG GGAGCTGCAG CAGAGCTTTT
2451 CGGGAACTTT GGCTTTGAAT GGCGGGGATC TTCTCGTAGC TATAATGTAG
2501 ATGCGGGTAG CAAAATCAAA TTTTAG
```

The PSORT algorithm predicts outer membrane (0.92).

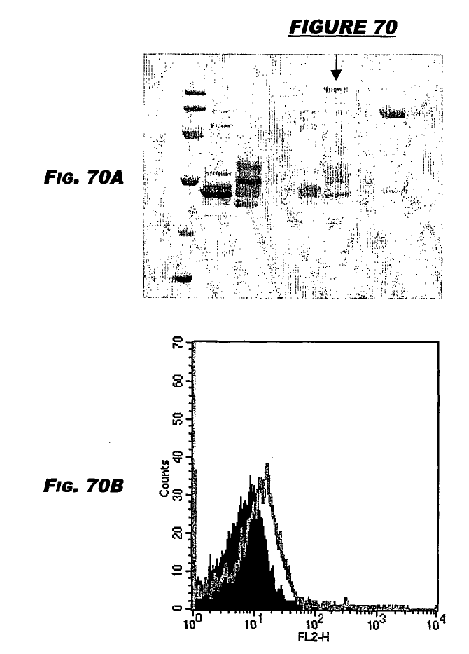

The protein was expressed in E. coli and purified as a GST-fusion product (FIG. 70A). The recombinant protein was used to immunize mice, whose sera were used in a Western blot and for FACS analysis (FIG. 70B).

The cp6270 protein was also identified in the 2D-PAGE experiment (Cpn0013).

These experiments show that cp6270 is a surface-exposed and immunoaccessible protein, and that it is a useful immunogen. These properties are not evident from the sequence alone.

### Example 71

The following C. pneumoniae protein (PID 4376402) was expressed <SEQ ID 141; cp6402>:

```
    1 MNVADLLSHL ETLLSSKIFQ DYGPNGLQVG DPQTPVKKIA VAVTADLETI
  51 KQAVAAEANV LIVHHGIFWK GMPYPITGMI HKRIQLLIEH NIQLIAYHLP
101 LDAHPTLGNN WRVALDLNWH DLKPFGSSLP YLGVQGSFSP IDIDSFIDLL
151 SQYYQAPLKG SALGGPSRVS SAALISGGAY RELSSAATSQ VDCFITGNFD
201 EPAWSTALES NINFLAFGHT ATEKVGPKSL AEHLKSEFPI STTFIDTANP
251 F*
```

The cp6402 nucleotide sequence <SEQ ID 142> is:

```
    1 ATGAATGTTG CGGATCTCCT TTCTCATCTT GAGACTCTTC TCTCATCAAA
  51 AATATTTCAG GATTATGGAC CCAACGGACT TCAAGTTGGA GATCCCCAAA
101 CTCCGGTAAA GAAAATCGCT GTTGCAGTTA CCGCAGATCT AGAAACCATA
151 AAACAAGCTG TTGCGGCCGA AGCAAACGTT CTCATTGTAC ACCACGGAAT
201 TTTTTGGAAA GGTATGCCCT ATCCTATTAC CGGCATGATC CATAAGCGCA
251 TCCAATTACT AATAGAACAC AATATCCAAC TCATTGCCTA CCACCTTCCT
301 TTGGATGCTC ACCCTACCTT AGGAAATAAC TGGAGAGTTG CCCTGGATCT
351 AAATTGGCAT GACTTGAAGC CCTTTGGTTC TTCCCTCCCT TATTTAGGAG
401 TGCAAGGCTC TTTCTCTCCT ATCGATATAG ATTCTTTCAT TGACCTGTTA
451 TCTCAATATT ACCAAGCTCC CCTAAAAGGA TCTGCCTTGG GCGGCCCCTC
501 TAGAGTCTCC TCAGCAGCTC TGATCTCAGG AGGAGCTTAT AGAGAACTCT
551 CTTCGGCAGC CACGTCCCAA GTCGATTGCT TCATCACAGG AAATTTTGAT
601 GAACCTGCAT GGTCGACAGC TCTAGAAAGC AATATCAACT TCCTAGCATT
651 TGGACATACA GCCACAGAAA AAGTAGGTCC AAAATCTCTT GCAGAGCATC
701 TAAAAAGCGA ATTTCCTATT TCCACAACCT TTATAGATAC GGCCAACCCC
751 TTCTAA
```

The PSORT algorithm predicts cytoplasmic (0.158).

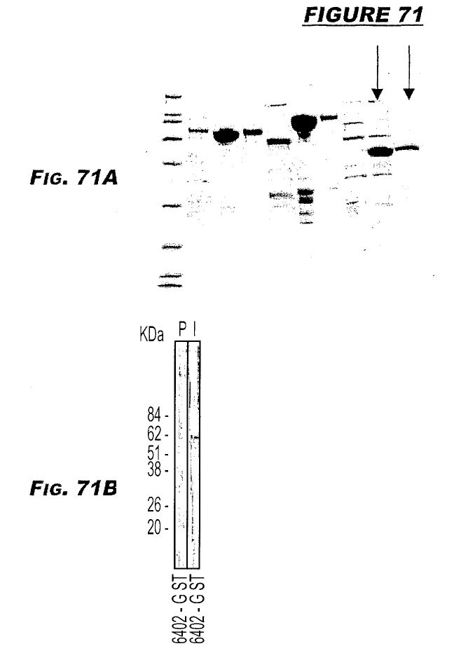

The protein was expressed in E. coli and purified as a GST-fusion product (FIG. 71A). The recombinant protein was used to immunize mice, whose sera were used in a Western blot (FIG. 71B) and for FACS analysis.

These experiments show that cp6402 is a surface-exposed and immunoaccessible protein, and that it is a useful immunogen. These properties are not evident from the sequence alone.

### Example 72

The following C. pneumoniae protein (PID 4376520) was expressed <SEQ ID 143; cp6520>:

```
    1 MKHYLSFSPS ADFFSKQGAI ETQVLFGERV LVKGSTCYAY SQLFHNELLW
  51 KPYPGHSFRS TLVPCTPEFH IHPNVSVVSV DAFLDPWGIP LPFGTLLHVN
101 SQNTVIFPKD ILNHMNTIWG SGTPQCDPRH LRRLNYNFFA ELLIKDADLL
151 LNFPYVWGGR SVHESLEKPG VDCSGFINIL YQAQGYNVPR NAADQYADCH
201 WISSFENLPS GGLIFLYPKE EKRISHVMLK QDSSTLIHAS GGGKKVEYFI
251 LEQDGKFLDS TYLFFRNNQR GRAFFGIPRK RKAFL*
```

The cp6520 nucleotide sequence <SEQ ID 144> is:

```
    1 ATGAAACACT ACCTATCATT TTCTCCTTCT GCTGATTTTT TCTCTAAACA
  51 GGGTGCTATT GAAACTCAAG TCCTTTTTGG AGAGCGCGTC TTAGTCAAAG
101 GGAGCACCTG CTATGCATAT TCCCAATTAT TCCACAATGA GCTGTTATGG
151 AAGCCCTATC CAGGTCATAG CTTTCGTTCT ACCCTAGTCC CCTGCACTCC
201 TGAATTTCAT ATCCATCCAA ATGTTTCTGT GGTTTCTGTG GATGCATTTT
251 TAGATCCTTG GGGGATCCCT CTTCCTTTTG GAACTTTACT CCATGTGAAT
301 TCTCAAAATA CCGTTATTTT CCCTAAGGAT ATTCTCAATC ATATGAACAC
351 CATCTGGGGC TCCGGCACAC CTCAATGCGA TCCTAGACAT CTACGTCGTC
401 TAAATTATAA CTTCTTTGCT GAACTTTTAA TTAAAGACGC AGACCTTTTA
451 CTGAACTTTC CCTATGTATG GGGAGGACGG TCTGTACACG AAAGTCTGGA
501 AAAGCCGGGT GTTGATTGTT CGGGATTTAT CAATATCCTT TACCAGGCAC
551 AGGGATACAA CGTCCCTAGA AACGCTGCAG ATCAATATGC GGATTGTCAT
601 TGGATCTCTA GCTTTGAGAA CCTTCCTTCT GGTGGGTTAA TATTTCTTTA
651 CCCTAAAGAA GAAAAGCGTA TTTCTCATGT TATGTTGAAA CAGGATAGTT
701 CCACCCTCAT TCATGCTTCT GGTGGAGGGA AAAAAGTGGA GTATTTCATT
751 TTAGAACAAG ATGGGAAGTT TTTAGATTCG ACTTATCTAT TTTTTAGAAA
801 TAATCAGAGG GGACGGGCAT TTTTTGGGAT CCCTAGAAAA AGAAAAGCCT
851 TTCTGTAA
```

The PSORT algorithm predicts cytoplasmic (0.265).

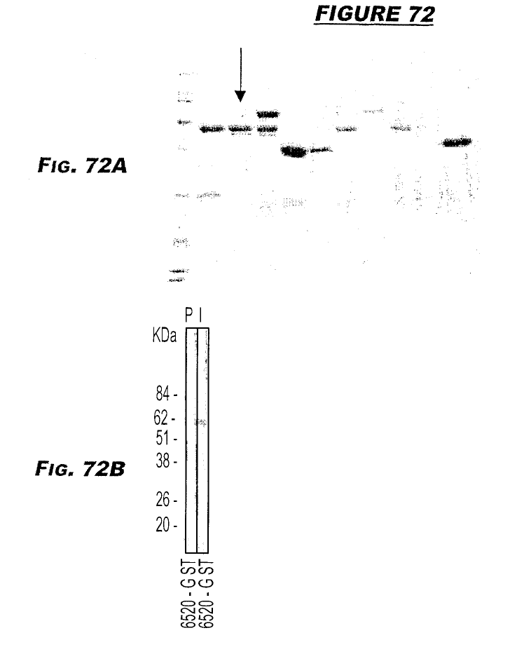

The protein was expressed in E. coli and purified as a GST-fusion product (FIG. 72A). The recombinant protein was used to immunize mice, whose sera were used in a Western blot (FIG. 72B) and for FACS analysis.

These experiments show that cp6520 is a surface-exposed and immunoaccessible protein, and that it is a useful immunogen. These properties are not evident from the sequence alone.

### Example 73

The following C. pneumoniae protein (PID 4376567) was expressed <SEQ ID 145; cp6567>:

```
    1 MTSPIPFQSS GDASFLAEQP QQLPSTSESQ LVTQLLTMMK HTQALSETVL
  51 QQQRDRLPTA SIILQVGGAP TGGAGAPFQP GPADDHHHPI PPPVVPAQIE
101 TEITTIRSEL QLMRSTLQQS TKGARTGVLV VTAILMTISL LAIIIIILAV
151 LGFTGVLPQV ALLMQGETNL IWAMVSGSII CFIALIGTLG LILTNKNTPL
201 PAS*
```

The cp6567 nucleotide sequence <SEQ ID 146> is:

```
    1 ATGACCTCAC CGATCCCCTT TCAGTCTAGT GGCGATGCCT CTTTCCTTGC
  51 CGAGCAGCCA CAGCAACTCC CGTCTACTTC TGAATCTCAG CTAGTAACTC
101 AATTGCTAAC CATGATGAAG CATACTCAAG CATTATCCGA AACGGTTCTT
151 CAACAACAAC GCGATCGATT ACCAACCGCA TCTATTATCC TTCAAGTAGG
201 AGGAGCTCCT ACAGGAGGAG CGGGTGCGCC TTTTCAACCA GGACCGGCAG
251 ATGATCATCA TCATCCCATA CCGCCGCCTG TTGTACCAGC TCAAATAGAA
301 ACAGAAATCA CCACTATAAG ATCCGAGTTA CAGCTCATGC GATCTACTCT
351 ACAACAAAGC ACAAAAGGAG CTCGTACAGG AGTTCTAGTG GTTACTGCAA
401 TCTTAATGAC GATCTCCTTA TTGGCTATTA TTATCATAAT ACTAGCTGTG
451 CTTGGATTTA CGGGCGTCTT GCCTCAAGTA GCTTTATTGA TGCAGGGTGA
501 AACAAATCTG ATTTGGGCTA TGGTGAGCGG TTCTATTATT TGCTTTATTG
551 CGCTAATTGG AACTCTAGGA TTAATTTTAA CAAATAAGAA CACGCCTCTA
601 CCGGCTTCTT AA
```

The PSORT algorithm predicts inner membrane (0.694).

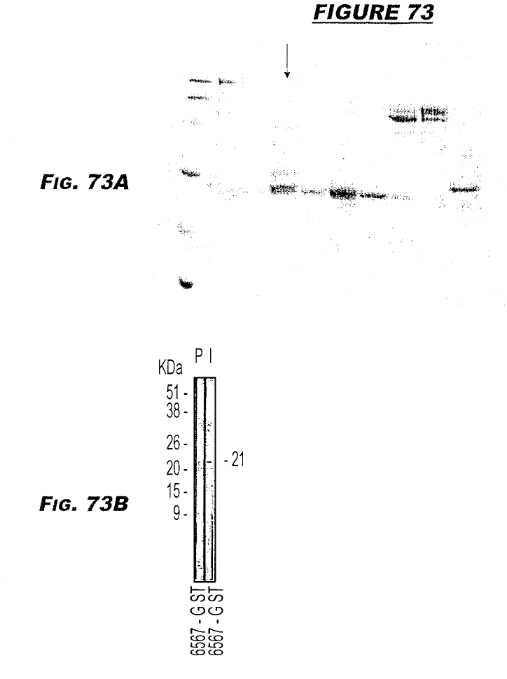

The protein was expressed in E. coli and purified as a GST-fusion product (FIG. 73A). The recombinant protein was used to immunize mice, whose sera were used in a Western blot (FIG. 73B) and for FACS analysis.

These experiments show that cp6567 is a surface-exposed and immunoaccessible protein, and that it is a useful immunogen. These properties are not evident from the sequence alone.

### Example 74

The following C. pneumoniae protein (PID 4376576) was expressed <SEQ ID 147; cp6576>:

```
    1 MLIMRNKVIL QISILALIQT PLTLFSTEKV KEGHVVVDSI TIITEGENAS
  51 NKHPLPKLKT RSGALFSQLD FDEDLRILAK EYDSVEPKVE FSEGKTNIAL
101 HLIAKPSIRN IHISGNQVVP EHKILKTLQI YRNDLFEREK FLKGLDDLRT
151 YYLKRGYFAS SVDYSLEHNQ EKGHIDVLIK INEGPCGKIK QLTFSGISRS
201 EKSDIQEFTQ TKQHSTTTSW FTGAGLYHPD IVEQDSLAIT NYLHNNGYAD
251 AIVNSHYDLD DKGNILLYMD IDRGSRYTLG HVHIQGFEVL PKRLIEKQSQ
301 VGPNDLYCPD KIWDGAHKIK QTYAKYGYIN TNVDVLFIPH ATRPIYDVTY
351 EVSEGSPYKV GLIKITGNTH TKSDVILHET SLFPGDTFNR LKLEDTEQRL
401 RNTGYFQSVS VYTVRSQLDP MGNADQYRDI FVEVKETTTG NLGLFLGFSS
451 LDNLFGGIEL SESNEDLEGA RNIFSKGFRC LRGGGEHLFL KANFGDKVTD
501 YTLKWTKPHF LNTPWTLGIE LDKSINRALS KDYAVQTYGG NVSTTYILNE
551 HLKYGLFYRG SQTSLHEKRK FLLGPNIDSN KGFVSAAGVN LNYDSVDSPR
601 TPTTGIRGGV TFEVSGLGGT YHFTKLSLNS SIYRKLTRKG ILKIKGEAQF
651 IKPYSNTTAE GVPVSERFFL GGETTVRGYK SFIIGEKYSA TEPQGGLSSL
701 LISEEFQYPL IRQPNISAFV FLDSGFVGLQ EYKISLKDLR SSAGFGLRFD
751 VMNNVPVMLG FGWPFRPTET LNGEKIDVSQ RFFFALGGMF *
```

A predicted signal peptide is highlighted.

The cp6576 nucleotide sequence <SEQ ID 148> is:

```
      1 ATGCTCATCA TGCGAAATAA AGTTATCTTG CAAATATCTA TTCTAGCGTT
    51 AATCCAAACC CCTTTAACTT TATTTTCTAC TGAAAAAGTT AAAGAAGGCC
  101 ATGTGGTGGT AGACTCTATC ACAATCATAA CGGAAGGAGA AAATGCTTCA
  151 AATAAACATC CCTTACCCAA ATTAAAGACC AGAAGTGGGG CTCTTTTTTC
  201 TCAATTAGAT TTTGATGAAG ACTTGAGAAT TCTAGCTAAA GAATACGACT
  251 CTGTTGAGCC TAAAGTAGAA TTTTCTGAAG GGAAAACTAA CATAGCCCTT
  301 CACCTAATAG CTAAACCCTC AATTCGAAAT ATTCATATCT CAGGAAATCA
  351 AGTCGTTCCT GAACATAAAA TTCTTAAAAC CCTACAAATT TACCGTAATG
  401 ATCTCTTTGA ACGAGAAAAA TTTCTTAAGG GTCTTGATGA TCTAAGAACG
  451 TATTATCTCA AGCGAGGATA TTTCGCATCC AGTGTAGACT ACAGTCTGGA
  501 ACACAATCAA GAAAAAGGTC ACATCGATGT TTTAATTAAA ATCAATGAAG
  551 GTCCTTGCGG GAAAATTAAA CAGCTTACGT TCTCAGGAAT CTCTCGATCA
  601 GAAAAATCAG ATATCCAAGA ATTTATTCAA ACCAAGCAGC ACTCTACAAC
  651 TACAAGTTGG TTTACTGGAG CTGGACTCTA TCACCCAGAT ATTGTTGAAC
  701 AAGATAGCTT GGCAATTACG AATTACCTAC ATAATAACGG GTACGCTGAT
  751 GCTATAGTCA ACTCTCACTA TGACCTTGAC GACAAAGGGA ATATTCTTCT
  801 TTACATGGAT ATTGATCGAG GGTCGCGATA TACCTTAGGA CACGTCCATA
  851 TCCAAGGGTT TGAGGTTTTG CCAAAACGCC TTATAGAAAA GCAATCCCAA
  901 GTCGGCCCCA ATGATCTTTA TTGCCCCGAT AAAATATGGG ATGGGGCTCA
  951 TAAGATCAAA CAAACTTATG CAAAGTATGG CTACATCAAT ACCAATGTAG
1001 ACGTTCTCTT CATCCCTCAC GCAACCCGCC CTATTTATGA TGTAACTTAT
1051 GAGGTAAGTG AAGGGTCTCC TTATAAAGTT GGGTTAATTA AAATTACTGG
1101 GAATACCCAT ACAAAATCTG ACGTTATTTT ACACGAAACC AGTCTCTTCC
1151 CAGGAGATAC ATTCAATCGC TTAAAGCTAG AAGATACTGA GCAACGTTTA
1201 AGAAATACAG GCTACTTCCA AAGCGTTAGT GTCTATACAG TTCGTTCTCA
1251 ACTTGATCCT ATGGGCAATG CGGATCAATA CCGAGATATT TTTGTAGAAG
1301 TCAAAGAAAC AACAACAGGA AACTTAGGCT TATTCTTAGG ATTTAGTTCT
1351 CTTGACAATC TTTTTGGAGG AATTGAACTA TCTGAAAGTA ATTTTGATCT
1401 ATTTGGAGCT AGAAATATAT TTTCTAAAGG TTTTCGTTGT CTAAGAGGCG
1451 GTGGAGAACA TCTATTCTTA AAAGCCAACT TCGGGGACAA AGTCACAGAC
1501 TATACTTTGA AGTGGACCAA ACCTCATTTT CTAAACACTC CTTGGATTTT
1551 AGGAATTGAA TTAGATAAAT CAATTAACAG AGCATTATCT AAAGATTATG
1601 CTGTCCAAAC CTATGGCGGG AACGTCAGCA CAACGTATAT CTTGAACGAA
1651 CACCTGAAAT ACGGTCTATT TTATCGAGGA AGTCAAACGA GTTTACATGA
1701 AAAACGTAAG TTCCTCCTAG GGCCAAATAT AGACAGCAAT AAAGGATTTG
1751 TCTCTGCTGC AGGTGTCAAC TTGAATTACG ATTCTGTAGA TAGTCCTAGA
1801 ACTCCAACTA CAGGGATTCG CGGGGGGGTG ACTTTTGAGG TTTCTGGTTT
1851 GGGAGGAACT TATCATTTTA CAAAACTCTC TTTAAACAGC TCTATCTATA
1901 GAAAACTTAC GCGTAAAGGT ATTTTGAAAA TCAAAGGGGA AGCTCAATTT
1951 ATTAAACCCT ATAGCAATAC TACAGCTGAA GGAGTTCCTG TCAGTGAGCG
2001 CTTCTTCCTA GGTGGAGAGA CTACAGTTCG GGGATATAAA TCCTTTATTA
2051 TCGGTCCAAA ATACTCTGCT ACAGAACCTC AGGGAGGACT CTCTTCGCTC
2101 CTTATTTCAG AAGAGTTTCA ATACCCTCTC ATCAGACAAC CTAATATTAG
2151 TGCCTTTGTA TTCTTAGACT CAGGTTTTGT CGGTTTACAA GAGTATAAGA
2201 TTTCGTTAAA AGATCTACGT AGTAGTGCTG GATTTGGTCT GCGCTTCGAT
2251 GTAATGAATA ATGTTCCTGT TATGTTAGGA TTTGGTTGGC CCTTCCGTCC
2301 AACCGAGACT TTGAATGGAG AAAAAATTGA TGTATCTCAG CGATTCTTCT
2351 TTGCTTTAGG GGGCATGTTC TAA
```

The PSORT algorithm predicts outer membrane (0.7658).

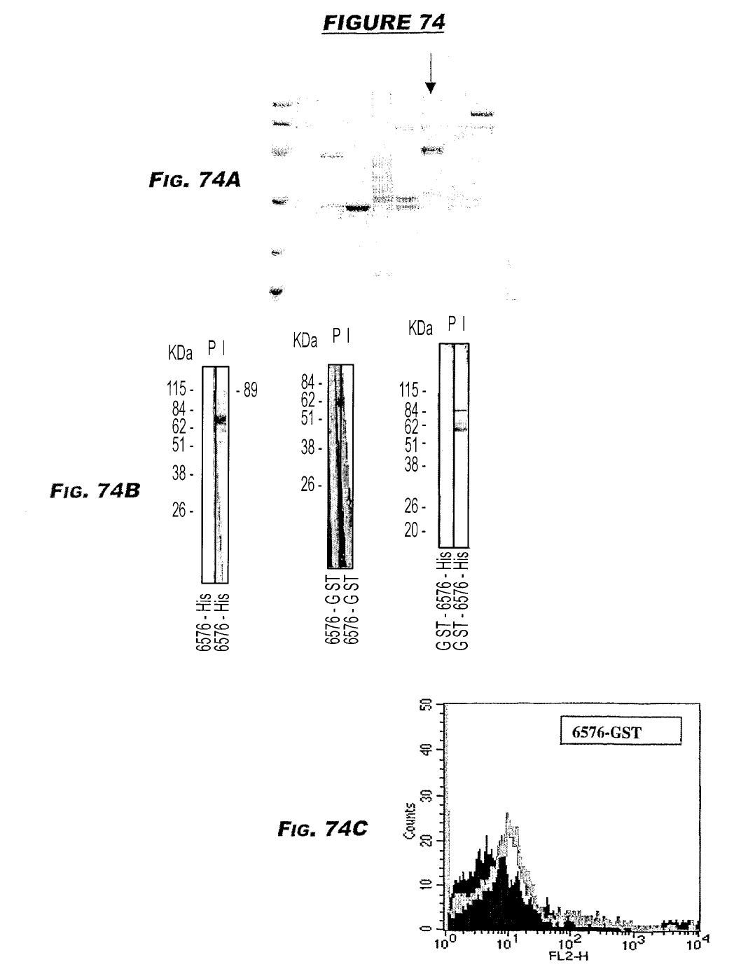

The protein was expressed in E. coli and purified as GST-fusion (FIG. 74A), his-tag and his-tag/GST-fusion products. The recombinant proteins were used to immunize mice, whose sera were used in a Western blot (FIG. 74B) and for FACS analysis (FIG. 74C).

The cp6576 protein was also identified in the 2D-PAGE experiment (Cpn0300).

These experiments show that cp6576 is a surface-exposed and immunoaccessible protein, and that it is a useful immunogen. These properties are not evident from the sequence alone.

### Example 75

The following C. pneumoniae protein (PID 4376607) was expressed <SEQ ID 149; cp6607>:

```
    1 MNKRQKDKLK ICVIISTLIL VGIFAPAPRG DTFKTFLKSE EAIIYSNQCN
  51 EDMRKILCDA IEHADEEIFL RIYNLEEPHI QQSLTRQAQA KNKVTIYYQK
101 FKIPQILKQA SNVTLVEQPP AGRKLMHQKA LSIDKKDAWL GSANYTNLSL
151 RLDNNLILGM HSSELCDLII TNTSGDFSIK DQTGKYFVLP QDRKIAIQAV
201 LEKIQTAQKT IQVAMFALTH SEIIQALHQA KQRGIHVDII IDRSHSKLTF
251 KQLRQLNINK DFVSINTAPC TLHHKFAVID NKTLLAGSIN WSKGRFSLND
301 ESLIILENLT KQQNQKLRMI WKDLAKHSEH PTVDDEEKEI IEKSLPVEEQ
351 EAA*
```

A predicted signal peptide is highlighted.

The cp6607 nucleotide sequence <SEQ ID 150> is:

```
      1 ATGAATAAAA GACAAAAAGA TAAATTAAAA ATCTGTGTTA TTATTAGCAC
    51 GTTGATTTTA GTAGGAATTT TTGCAAGAGC TCCTCGTGGT GACACTTTTA
  101 AGACTTTTTT AAAGTCTGAA GAAGCTATCA TCTACTCAAA TCAATGCAAT
  151 GAGGACATGC GTAAAATTCT ATGCGATGCT ATAGAACACG CTGATGAAGA
  201 GATCTTCCTA CGTATTTATA ACCTCTCAGA ACCCAAGATC CAACAGAGTT
  251 TAACTCGACA AGCTCAAGCA AAAAACAAAG TTACGATCTA CTATCAAAAA
  301 TTTAAAATTC CCCAAATCTT AAAGCAAGCC AGCAATGTAA CTTTAGTCGA
  351 GCAACCTCCA GCAGGGCGTA AACTGATGCA TCAAAAAGCT CTTTCCATAG
  401 ATAAGAAAGA TGCTTGGCTA GGATCTGCGA ACTACACCAA TCTTTCTCTA
  451 CGTTTAGATA ATAATCTCAT TCTAGGAATG CATAGCTCGG AGCTCTGTGA
  501 TCTCATTATC ACAAATACCT CTGGAGACTT TTCTATAAAG GATCAAACAG
  551 GAAAGTATTT TGTTCTTCCT CAAGATCGTA AAATTGCAAT ACAAGCTGTA
  601 CTCGAAAAAA TCCAGACAGC TCAGAAAACC ATCCAAGTTG CTATGTTTGC
  651 TCTGACCCAC TCGGAGATTA TTCAAGCCTT ACATCAAGCA AAACAACGAG
  701 GAATCCATGT AGATATTATC ATTGATAGAA GTCATAGCAA ACTTACTTTT
  751 AAGCAATTAC GACAATTAAA TATCAATAAA GACTTTGTTT CTATAAATAC
  801 CGCACCCTGT ACTCTTCACC ATAAGTTTGC AGTTATAGAT AATAAAACTC
  851 TACTTGCAGG ATCTATAAAT TGGTCTAAAG GAAGATTCTC CTTAAATGAT
  901 GAAAGCTTGA TCATACTGGA AAACCTGACC AAACAACAAA ATCAGAAACT
  951 TCGAATGATT TGGAAAGATC TAGCTAAGCA TTCAGAACAT CCTACAGTAG
1001 ACGATGAAGA AAAAGAAATT ATAGAAAAAA GTCTTCCAGT AGAAGAGCAA
1051 GAAGCAGCGT GA
```

The PSORT algorithm predicts periplasmic (0.934).

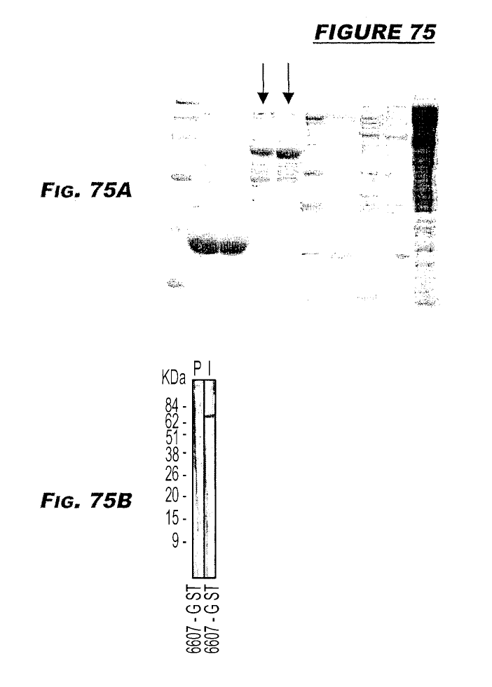

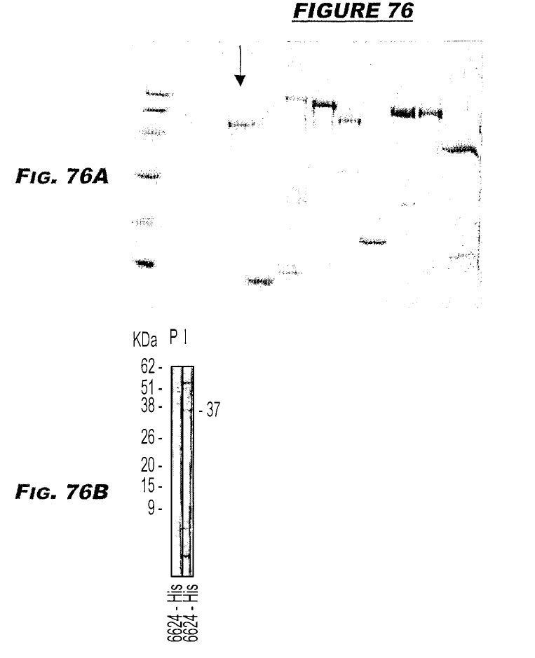

The protein was expressed in E. coli and purified as a his-tagged product (FIG. 75A) and also as a GST-fusion. The GST-fusion protein was used to immunize mice, whose sera were used in a Western blot (FIG. 75B) and for FACS analysis.

These experiments show that cp6607 is a surface-exposed and immunoaccessible protein, and that it is a useful immunogen. These properties are not evident from the sequence alone.

### Example 76

The following C. pneumoniae protein (PID 4376624) was expressed <SEQ ID 151; cp6624>:

```
    1 MDAKMGYIFK VMRWIFCFVA CGITFGCINS GFQNANSRPC ILSMNRMIHD
  51 CVERVVGNRL ATAVLIKGSL DPHAYEMVKG DKDKIAGSAV IFCNGLGLEH
101 TLSLRKHLEN NPNSVKLGER LIARGAFVPL EEDGICDPHI WMDLSIWKEA
151 VIEITEVLIE KFPEWSAEFK ANSEELVCEM SILDSWAKQC LSTIPENLRY
201 LVSGHNAFSY FTRRYLATPE EVASGAWRSR CISPEGLSPE AQISVRDIMA
251 VVDYINEHDV SVVFPEDTLN QDALKKIVSS LKKSHLVRLA QKPLYSDNVD
301 DNYFSTFKHN VCLITEELGG VALECQR*
```

The cp6624 nucleotide sequence <SEQ ID 152> is:

```
    1 ATGGATGCGA AAATGGGATA TATATTTAAA GTGATGCGTT GGATTTTCTG
  51 TTTCGTGGCA TGTGGTATAA CTTTTGGATG TACCAATTCT GGGTTTCAGA
101 ATGCAAATTC ACGTCCTTGT ATACTATCCA TGAATCGCAT GATTCATGAT
151 TGTGTTGAAA GAGTCGTGGG GAATAGGCTT GCTACCGCTG TTTTGATCAA
201 AGGATCCTTA GACCCTCATG CGTATGAGAT GGTTAAAGGG GATAAGGACA
251 AGATTGCTGG AAGTGCCGTA ATTTTTTGTA ACGGCCTGGG TCTTGAGCAT
301 ACATTAAGTT TGCGGAAGCA TTTAGAAAAT AATCCCAATA GTGTCAAGTT
351 AGGGGAGCGG TTGATAGCGC GTGGGGCCTT TGTTCCTCTA GAAGAAGACG
401 GTATTTGCGA TCCTCATATC TGGATGGATC TTTCTATTTG GAAGGAAGCT
451 GTCATAGAAA TTACAGAAGT TCTCATTGAA AAGTTCCCTG AATGGTCTGC
501 TGAATTTAAA GCAAATAGTG AGGAACTTGT TTGTGAAATG TCTATTTTAG
551 ATTCTTGGGC GAAACAATGC TTGAGCACAA TTCCTGAAAA TTTACGGTAT
601 CTTGTCTCAG GTCATAATGC GTTCAGTTAC TTTACACGTC GCTATTTAGC
651 TACTCCTGAA GAAGTGGCTT CCGGAGCATG GAGGTCTCGT TGTATTTCTC
701 CTGAGGGTCT ATCTCCAGAA GCTCAAATCA GTGTTCGTGA TATTATGGCG
751 GTTGTAGATT ATATTAATGA GCATGATGTC AGTGTGGTTT TCCCTGAGGA
801 TACTCTGAAC CAAGATGCGT TGAAAAAAAT TGTTTCTTCT CTGAAGAAAA
851 GTCATTTAGT TCGTCTAGCT CAAAAACCAT TGTATAGTGA TAATGTGGAC
901 GACAATTATT TTAGCACCTT TAAACATAAT GTCTGCCTTA TCACAGAAGA
951 ATTAGGAGGG GTGGCTCTTG AATGTCAAAG ATGA
```

The PSORT algorithm predicts inner membrane (0.168).

The protein was expressed in E. coli and purified as a his-tag product (FIG. 76A). The recombinant protein was used to immunize mice, whose sera were used in a Western blot (FIG. 76B) and for FACS analysis.

The cp6624 protein was also identified in the 2D-PAGE experiment.

These experiments show that cp6624 is a surface-exposed and immunoaccessible protein, and that it is a useful immunogen. These properties are not evident from the sequence alone.

### Example 77

The following C. pneumoniae protein (PID 4376728) was expressed <SEQ ID 153; cp6728>:

```
    1 MKSSVSWLFF SSIPLFSSLS IVAAEVTLDS SNNSYDGSNG TTFTVFSTTD
  51 AAAGTTYSLL SDVSFQNAGA LGIPLASGCF LEAGGDLTFQ GNQHALKFAF
101 INAGSSAGTV ASTSAADKNL LFNDFSRLSI ISCPSLLLSP TGQCALKSVG
151 NLSLTGNSQI IFTQNFSSDN GGVINTKNFL LSGTSQFASF SRNQAFTGKQ
201 GGVVYATGTI TIENSPGIVS FSQNLAKGSG GALYSTDNCS ITDNFQVIFD
251 GNSAWEAAQA QGGAICCTTT DKTVTLTGNK NLSFTNNTAL TYGGAISGLK
301 VSISAGGPTL FQSNISGSSA GQGGGGAINI ASAGELALSA TSGDITFNNN
351 QVTNGSTSTR NAINIIDTAK VTSTRAATGQ SIYFYDPITN PGTAASTDTL
401 NLNLADANSE IEYGGAIVES GEKLSPTEKA IAANVTSTTR QPAVLARGDL
451 VLRDGVTVTF KDLTQSPGSR ILMDGGTTLS AKEANLSLNG LAVNLSSLDG
501 TNKAALKTEA ADKNISLSGT IALIDTEGSF YENHNLKSAS TYPLLELTTA
551 GANGTITLGA LSTLTLQEPE THYGYQGNWQ LSWANATSSK IGSINWTRTG
601 YIPSPERKSN LPLNSLWGNF IDIRSTNQLI ETKSSGEPFE RELWLSGIAN
651 FFYRDSMPTR HGFRHISGGY ALGITATTPA EDQLTFAFCQ LFARDRNHTT
701 GKNHGDTYGA SLYFHHTEGL FDIANFLWGK ATPAPWVLSE ISQIIELSED
751 AKFSYLHTDN HMKTYYTDNS IIKGSWRNDA FCADLGASLP FVISVPYLLK
801 EVEPFVKVQY IYAHQQDFYE RHAEGPAFNK SELINVEIPI GVTFERDSKS
851 EKGTYDLTLM YILDAYRRNP KCQTSLIASD ANWMAYGTNL ARQGFSVPAA
901 NHFQVNPHME IFGQFAFEVR SSSRNYNTNL GSKFCF*
```

The cp6728 nucleotide sequence <SEQ ID 154> is:

```
      1 ATGAAGTCCT CTGTCTCTTG GTTGTTCTTT TCTTCAATCC CGCTCTTTTC
    51 ATCGCTCTCT ATAGTCGCGG CAGAGGTGAC CTTAGATAGC AGCAATAATA
  101 GCTATGATGG ATCTAACGGA ACTACCTTCA CGGTCTTTTC CACTACGGAC
  151 GCTGCTGCAG GAACTACCTA TTCCTTACTT TCCGACGTAT CCTTTCAAAA
  201 TGCAGGGGCT TTAGGAATTC CCTTAGCCTC AGGATGCTTC CTAGAAGCGG
  251 GCGGCGATCT TACTTTCCAA GGAAATCAAC ATGCACTGAA GTTTGCATTT
  301 ATCAATGCGG GCTCTAGCGC TGGAACTGTA GCCAGTACCT CAGCAGCAGA
  351 TAAGAATCTT CTCTTTAATG ATTTTTCTAG ACTCTCTATT ATCTCTTGTC
  401 CCTCTCTTCT TCTCTCTCCT ACTGGACAAT GTGCTTTAAA ATCTGTGGGG
  451 AATCTATCTC TAACTGGCAA TTCCCAAATT ATATTTACTC AGAACTTCTC
  501 GTCAGATAAC GGCGGTGTTA TCAATACGAA AAACTTCTTA TTATCAGGGA
  551 CATCTCAGTT TGCGAGCTTT TCGAGAAACC AAGCCTTCAC AGGGAAGCAA
  601 GGCGGTGTAG TTTACGCTAC AGGAACTATA ACTATCGAGA ACAGCCCTGG
  651 GATAGTTTCC TTCTCTCAAA ACCTAGCGAA AGGATCTGGC GGTGCTCTGT
  701 ACAGCACTGA CAACTGTTCG ATTACAGATA ACTTTCAAGT GATCTTTGAC
  751 GGCAATAGTG CTTGGGAAGC CGCTCAAGCT CAGGGCGGGG CTATTTGTTG
  801 CACTACGACA GATAAAACAG TGACTCTTAC TGGGAACAAA AACCTCTCTT
  851 TCACAAATAA TACAGCATTG ACATATGGCG GAGCCATCTC TGGACTCAAG
  901 GTCAGTATTT CCGCTGGAGG TCCTACTCTA TTTCAAAGTA ATATCTCAGG
  951 AAGTAGCGCC GGTCAGGGAG GAGGAGGAGC GATCAATATA GCATCTGCTG
1001 GGGAACTCGC TCTCTCTGCT ACTTCTGGAG ATATTACCTT CAATAACAAC
1051 CAAGTCACCA ACGGAAGCAC AAGTACAAGA AACGCAATAA ATATCATTGA
1101 TACCGCTAAA GTCACATCGA TACGAGCTGC TACGGGGCAA TCTATCTATT
1151 TCTATGATCC CATCACAAAT CCAGGAACCG CAGCTTCTAC CGACACATTG
1201 AACTTAAACT TAGCAGATGC GAACAGTGAG ATCGAGTATG GGGGTGCGAT
1251 TGTCTTTTCT GGAGAAAAGC TTTCCCCTAC AGAAAAAGCA ATCGCTGCAA
1301 ACGTCACCTC TACTATCCGA CAACCTGCAG TATTAGCGCG GGGAGATCTT
1351 GTACTTCGTG ATGGAGTCAC CGTAACTTTC AAGGATCTGA CTCAAAGTCC
1401 AGGATCCCGC ATCTTAATGG ATGGGGGGAC TACACTTAGT GCTAAAGAGG
1451 CAAATCTTTC GCTTAATGGC TTAGCAGTAA ATCTCTCCTC TTTAGATGGA
1501 ACCAACAAGG CAGCTTTAAA AACAGAAGCT GCAGATAAAA ATATCAGCCT
1551 ATCGGGAACG ATTGCGCTTA TTGACACGGA AGGGTCATTC TATGAGAATC
1601 ATAACTTAAA AAGTGCTAGT ACCTATCCTC TTCTTGAACT TACCACCGCA
1651 GGAGCCAACG GAACGATTAC TCTGGGAGCT CTTTCTACCC TGACTCTTCA
1701 AGAACCTGAA ACCCACTACG GGTATCAAGG AAACTGGCAG TTGTCTTGGG
1751 CAAATGCAAC ATCCTCAAAA ATAGGAAGCA TCAACTGGAC CCGTACAGGA
1801 TACATTCCTA GTCCTGAGAG AAAAAGTAAT CTCCCTCTAA ATAGCTTATG
1851 GGGAAACTTT ATAGATATAC GCTCGATCAA TCAGCTTATA GAAACCAAGT
1901 CCAGTGGGGA GCCTTTTGAG CGTGAGCTAT GGCTTTCAGG AATTGCGAAT
1951 TTCTTCTATA GAGATTCTAT GCCCACCCGC CATGGTTTCC GCCATATCAG
2001 CGGGGGTTAT GCACTAGGGA TCACAGCAAC AACTCCTGCC GAGGATCAGC
2051 TTACTTTTGC CTTCTGCCAG CTCTTTGCTA GAGATCGCAA TCATATTACA
2101 GGTAAGAACC ACGGAGATAC TTACGGTGCC TCTTTGTATT TCCACCATAC
2151 AGAAGGGCTC TTCGACATCG CCAATTTCCT CTGGGGAAAA GCAACCCGAG
2201 CTCCCTGGGT GCTCTCTGAG ATCTCCCAGA TCATTCCTTT ATCGTTCGAT
2251 GCTAAATTCA GTTATCTCCA TACAGACAAC CACATGAAGA CATATTATAC
2301 CGATAACTCT ATCATCAAGG GTTCTTGGAG AAACGATGCC TTCTGTGCAG
2351 ATCTTGGAGC TAGCCTGCCT TTTGTTATTT CCGTTCCGTA TCTTCTGAAA
2401 GAAGTCGAAC CTTTTGTCAA AGTACAGTAT ATCTATGCGC ATCAGCAAGA
2451 CTTCTACGAG CGTCATGCTG AAGGACGCGC TTTCAATAAA AGCGAGCTTA
2501 TCAACGTAGA GATTCCTATA GGCGTCACCT TCGAAAGAGA CTCAAAATCA
2551 GAAAAGGGAA CTTACGATCT TACTCTTATG TATATACTCG ATGCTTACCG
2601 ACGCAATCCT AAATGTCAAA CTTCCCTAAT AGCTAGCGAT GCTAACTGGA
2651 TGGCCTATGG TACCAACCTC GCACGACAAG GTTTTTCTGT TCGTGCTGCG
2701 AACCATTTCC AAGTGAACCC CCACATGGAA ATCTTCGGTC AATTCGCTTT
2751 TGAAGTACGA AGTTCTTCAC GAAATTATAA TACAAACCTA GGCTCTAAGT
2801 TTTGTTTCTA G
```

The PSORT algorithm predicts inner membrane (0.187).

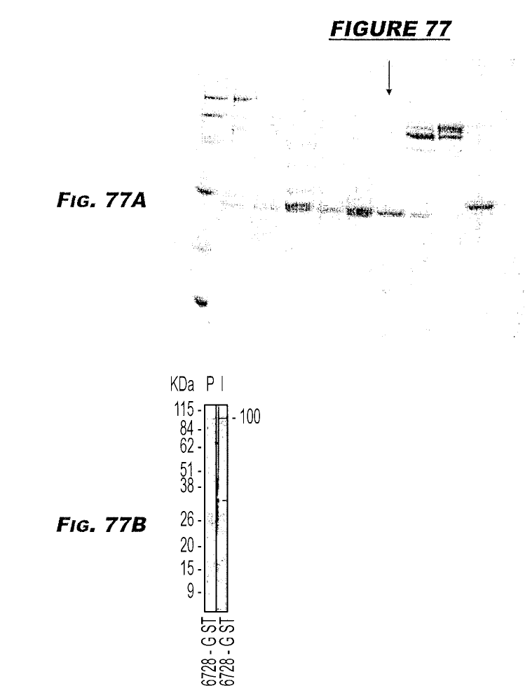

The protein was expressed in E. coli and purified as a GST-fusion product (FIG. 77A). The recombinant protein was used to immunize mice, whose sera were used in a Western blot (FIG. 77B) and for FACS analysis.

The cp6728 protein was also identified in the 2D-PAGE experiment.

These experiments show that cp6728 is a surface-exposed and immunoaccessible protein, and that it is a useful immunogen. These properties are not evident from the sequence alone.

### Example 78

The following C. pneumoniae protein (PID 4376847) was expressed <SEQ ID 155; cp6847>:

```
    1 MFVMKKLVRL CVVLLSLLPN VLFSSDLLRE EGIKKMMDKL IEYHVDAQEV
  51 STDILSRSLS SYIQSFDPHK SYLSNQEVAV FLQSPETKKR LLKNYKAGNF
101 AIYRNINQLI HESILRARQW RNEWVKNPKE LVLEASSYQI SKQPMQWSKS
151 LDEVKQRQRA LLLSYLSLHL AGASSSRYEG KEEQLAALCL RQIENHENVY
201 LGINDHGVAM DRDEEAYQFH IRVVKALAHS LDAHTAYFSK DEALAMRIQL
251 EKGMCGIGVV LKEDIDGVVV REIIPGGPAA KSGDLQLGDI IYRVDGKDIE
301 HLSFRGVLDC LRGGHGSTVV LDIHRGESDH TIALRREKIL LEDRRVDVSY
351 EPYGDGVIGK VTLHSFYEGE NQVSSEQDLR RAIQGLKEKN LLGLVLDIRE
401 NTGGFLSQAI KVSGLFMTNG VVVVSRYADG TMKCYRTVSP KKFYDGPLAI
451 LVSKSSASAA EIVAQTLQDY GVALVVGDEQ TYGKGTIQHQ TITGDASQDD
501 CFKVTVGKYY SPSGKSTQLQ GVKSDILIPS LYAEDRLGER FLEHPLPADC
551 CDNVLHDPLT DLDTQTRPWF QKYYLPNLQK QETLWREMLP QLTKNSEQRL
501 SENSNFQAFL SQIKSSEKTD LSYGSNDLQL EESINILKDM ILLQQCRK*
```

A predicted signal peptide is highlighted.

The cp6847 nucleotide sequence <SEQ ID 156> is:

```
      1 ATGTTCGTAA TGAAAAAACT TGTCCGTCTA TGCGTAGTTC TTCTTTCTTT
    51 ACTTCCGAAT GTATTATTTT CTTCGGATCT TTTACGAGAA GAGGGCATCA
  101 AAAAGATGAT GGACAAGCTG ATCGAGTATC ATGTCGATGC TCAAGAGGTT
  151 TCTACGGATA TACTCTCGCG TTCTTTATCT AGTTACATTC AATCTTTTGA
  201 TCCTCATAAA TCTTATCTTT CAAACCAAGA GGTTGCAGTT TTTCTACAGT
  251 CTCCGGAAAC AAAGAAACGT CTCTTAAAGA ATTATAAGGC AGGCAACTTT
  301 GCTATTTATC GCAACATCAA TCAATTAATT CATGAGAGTA TTCTTCGTGC
  351 CAGGCAGTGG AGAAACGAAT GGGTTAAGAA TCCAAAAGAG CTTGTATTGG
  401 AGGCATCCTC ATATCAGATA TCGAAGCAAC CTATGCAATG GAGCAAATCT
  451 TTAGACGAAG TGAAGCAGAG ACAACGCGCT CTACTCCTTT CCTATCTTTC
  501 TTTACATCTT GCTGGAGCTT CTTCCTCTCG TTATGAGGGT AAAGAAGAGC
  551 AGCTTGCTGC TCTGTGTCTA CGTCAAATCG AGAACCATGA GAATGTATAT
  601 TTAGGTATCA ACGATCATGG TGTTGCTATG GATCGGGATG AAGAAGCCTA
  651 CCAATTCCAT ATCCGTGTTG TTAAAGCTTT AGCTCATAGC TTAGATGCAC
  701 ATACGGCGTA TTTCAGTAAG GACGAAGCGT TGGCGATGCG AATCCAACTA
  751 GAAAAAGGCA TGTGTGGAAT TGGTGTTGTT CTGAAGGAAG ATATTGATGG
  801 AGTTGTTGTT AGAGAAATCA TTCCTGGGGG ACCTGCGGCT AAATCTGGGG
  851 ATCTTCAGCT TGGAGATATC ATCTATCGGG TGGATGGCAA GGATATCGAG
  901 CATCTTTCTT TCCGCGGTGT TTTAGATTGT TTACGTGGAG GTCATGGCTC
  951 TACTGTAGTC TTAGATATCC ATCGTGGGGA GAGCGATCAT ACGATCGCCT
1001 TGAGAAGGGA GAAAATCCTT TTAGAAGACC GTCGTGTGGA TGTTTCCTAT
1051 GAGCCTTATG GAGATGGTGT GATTGGGAAA GTTACGTTAC ATTCTTTTTA
1101 TGAAGGAGAA AATCAGGTTT CTAGTGAACA AGATCTACGT CGAGCGATTC
1151 AGGGATTAAA GGAGAAGAAC CTTCTTGGAT TAGTTTTAGA TATCCGAGAA
1201 AATACGGGTG GATTTTTATC TCAAGCGATC AAAGTTTCTG GTTTATTTAT
1251 GACCAATGGC GTTGTGGTTG TATCTCGCTA TGCTGATGGT ACCATGAAGT
1301 GCTACCGCAC AGTATCTCCT AAAAAATTCT ATGATGGTCC TTTGGCTATT
1351 TTAGTATCTA AAAGTTCCGC ATCAGCAGCG GAGATTGTAG CACAAACTCT
1401 CCAAGATTAT GGAGTTGCTT TAGTTGTTGG AGATGAGCAG ACCTATGGGA
1451 AGGGAACGAT TCAGCATCAA ACAATTACTG GAGATGCCTC TCAGGACGAT
1501 TGTTTTAAGG TTACTGTAGG GAAATATTAT TCCCCTTCTG GGAAATCGAC
1551 TCAACTTCAG GGAGTAAAAT CCGATATTTT AATTCCTTCT CTCTATGCTG
1601 AAGATCGTCT AGGAGAGCGT TTTCTAGAGC ATCCCTTACC TGCAGATTGC
1651 TGTGATAATG TACTTCACGA TCCTCTCACG GACTTGGATA CTCAAACACG
1701 TCCTTGGTTT CAAAAATACT ATCTTCCTAA TCTACAAAAG CAAGAGACTC
1751 TTTGGAGAGA GATGCTACCT CAGCTTACGA AAAACAGTGA GCAAAGGCTT
1801 TCTGAGAATT CGAATTTTCA GGCATTTTTG TCGCAGATAA AATCATCTGA
1851 AAAAACGGAC CTATCCTATG GTTCCAATGA TTTACAATTG GAAGAGTCGA
1901 TAAACATTTT GAAGGACATG ATTTTATTAC AACAGTGTAG AAAATAA
```

The PSORT algorithm predicts periplasmic (0.932).

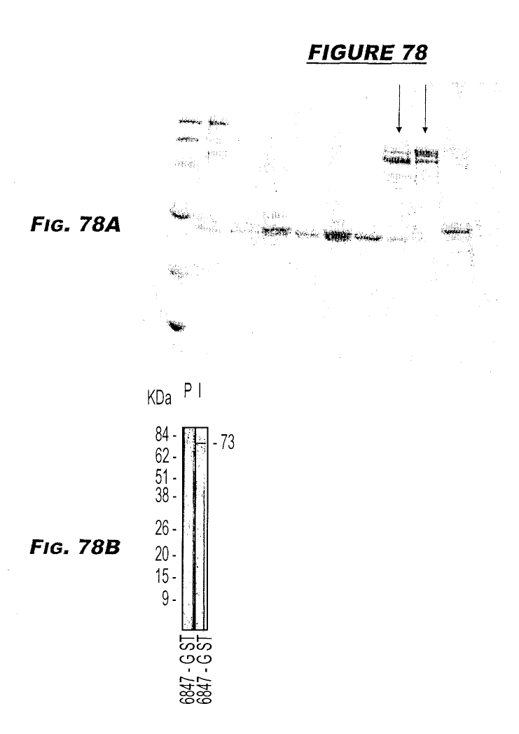

The protein was expressed in E. coli and purified as a GST-fusion product (FIG. 78A). The recombinant protein was used to immunize mice, whose sera were used in a Western blot (FIG. 78B) and for FACS analysis.

These experiments show that cp6847 is a surface-exposed and immunoaccessible protein, and that it is a useful immunogen. These properties are not evident from the sequence alone.

### Example 79

The following C. pneumoniae protein (PID 4376969) was expressed <SEQ ID 157; cp6969>:

| | |
|---|---|
| 1 | MRLFSLGTIY LFFSLALSSC CGYSILNSPY HLSSLGKSLL QERIFIAPIK |
| 51 | EDPHGQLCSA LTYELSKRSF AISGRSSCAG YTLKVELLNG IDKNIGFTYA |
| 101 | PNKLGDKTHR HFIVSNEGRL SLSAKVQLIN NDTQEVLIDQ CVARESVDFD |
| 151 | FEPDLGTANA HEFALGQFEM HSEAIKSARR ILSIRLAETI AQQVYYDLF* |

A predicted signal peptide is highlighted.

The cp6969 nucleotide sequence <SEQ ID 158> is:

| | |
|---|---|
| 1 | ATGAGATTGT TTTCTTTAGG CACGATTTAT CTTTTTTTTT CTCTAGCACT |
| 51 | TTCGTCATGC TGTGGTTACT CTATTTTAAA CAGCCCGTAT CACTTATCGT |
| 101 | CTTTAGGTAA GTCTTTATTA CAGGAAAGAA TTTTCATTGC TCCCATAAAA |
| 151 | GAAGATCCTC ATGGTCAGCT CTGCTCAGCT CTAACTTATG AGCTTAGTAA |
| 201 | GCGTTCTTTT GCTATCTCTG GAAGGAGTTC TTGCGCAGGC TATACTCTTA |
| 251 | AAGTAGAGCT TCTGAATGGT ATTGACAAGA ATATAGGTTT TACGTATGCC |
| 301 | CCAAATAAAC TCGGAGATAA GACTCACAGG CATTTTATAG TCTCTAATGA |
| 351 | AGGCAGACTA TCACTATCTG CAAAAGTACA GCTTATCAAT AATGACACTC |
| 401 | AAGAAGTCCT TATAGACCAA TGTGTTGCTC GAGAGTCTGT AGACTTTGAC |
| 451 | TTTGAGCCTG ACTTAGGAAC AGCAAACGCT CATGAATTTG CTTTAGGCCA |
| 501 | ATTTGAAATG CATAGTGAAG CCATAAAAAG TGCTCGCCGT ATACTATCTA |
| 551 | TACGCCTAGC CGAGACGATT GCTCAACAGG TATACTATGA CCTTTTTTGA |

The PSORT algorithm predicts inner membrane (0.126).

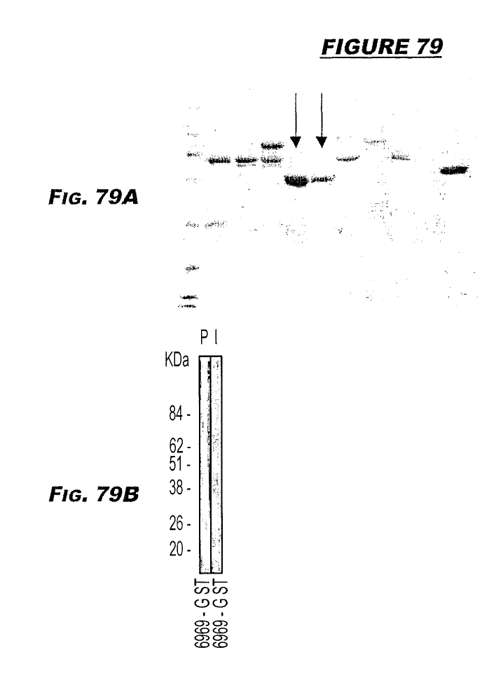

The protein was expressed in E. coli and purified as a GST-fusion product (FIG. 79A). The recombinant protein was used to immunize mice, whose sera were used in a Western blot (FIG. 79B) and for FACS analysis.

These experiments show that cp6969 is a surface-exposed and immunoaccessible protein, and that it is a useful immunogen. These properties are not evident from the sequence alone.

### Example 80

The following C. pneumoniae protein (PID 4377109) was expressed <SEQ ID 159; cp7109>:

| | |
|---|---|
| 1 | MKKTCCQNYR SIGVVFSVVL FVLTTQTLFA GHFIDIGTSG LYSWARGVSG |
| 51 | DGRVVVGYEG GNAFKYVDGE KFLLEGLVPR SEALVFKASY DGSVIIGISD |
| 101 | QDPSCRAVKW VNGALVDLGI FSEGMQSFAE GVSSDGKTIV GCLYSDDTET |
| 151 | NFAVKWDETG MVVLPNLPED RHSCAWDASE DGSVIVGDAM GSEEIAKAVY |
| 201 | WKDGEQHLLS NIPGAKRSSA HAVSKDGSFI VGEFISEENE VHAFVYHNGV |
| 251 | IKDIGTLGGD YSVATGVSRD GKVIVGHSTR TDGEYRAFKY VDGRMIDLGT |
| 301 | LGGSASFAFG VSDDGKTIVG KFETELGECH AFIYLDD* |

A predicted signal peptide is highlighted.

The cp7109 nucleotide sequence <SEQ ID 160> is:

| | |
|---|---|
| 1 | ATGAAAAAGA CATGTTGCCA AAATTACAGA TCGATAGGCG TTGTGTTCTC |
| 51 | TGTGGTACTT TTCGTTCTTA CAACACAGAC GCTGTTTGCA GGACATTTTA |
| 101 | TTGATATTGG AACTTCTGGA TTATATTCTT GGGCTCGAGG TGTATCTGGA |
| 151 | GATGGCCGCG TTGTCGTAGG TTATGAAGGT GGCAATGCAT TTAAATATGT |
| 201 | TGATGGTGAG AAATTTCTGT TAGAAGGTTT GGTCCCGAGA TCCGAGGCCT |
| 251 | TGGTATTTAA AGCTTCTTAT GATGGCTCTG TAATTATAGG AATCTCGGAT |
| 301 | CAAGATCCGT CTTGCCGCGC TGTGAAGTGG GTAAACGGTG CACTTGTTGA |
| 351 | TCTTGGAATA TTTTCTGAGG GAATGCAATC TTTTGCAGAG GGTGTTTCCA |
| 401 | GTGATGGAAA GACGATTGTA GGGTGCCTAT ATAGTGATGA TACAGAGACA |
| 451 | AACTTTGCTG TGAAGTGGGA TGAAACAGGA ATGGTTGTTC TCCCTAACTT |
| 501 | ACCAGAAGAT CGACATTCTT GCGCTTGGGA TGCCTCTGAA GATGGCTCTG |
| 551 | TGATTGTAGG GGACGCCATG GGTAGCGAGG AAATTGCCAA GGCAGTGTAC |
| 601 | TGGAAGGACG GTGAACAACA TCTGCTTTCT AATATCCCAG GAGCTAAAAG |
| 651 | ATCGTCAGCA CATGCAGTTT CTAAAGATGG ATCTTTTATC GTAGGCGAGT |
| 701 | TCATCAGTGA AGAAAATGAA GTTCATGCCT TTGTTTATCA CAACGGTGTT |
| 751 | ATCAAAGATA TCGGGACTTT AGGAGGAGAT TACTCTGTAG CAACTGGAGT |
| 801 | TTCTAGGGAT GGTAAGGTCA TCGTGGGTCA TTCTACAAGA ACAGATGGTG |
| 851 | AATACCGTGC ATTTAAATAT GTGGATGGAA GAATGATAGA TTTGGGGACT |
| 901 | TTAGGAGGTT CAGCATCTTT TGCTTTTGGT GTTTCTGACG ATGGCAAAAC |
| 951 | AATCGTAGGA AAATTTGAAA CAGAGCTAGG AGAATGTCAT GCCTTTATCT |
| 1001 | ACCTTGATGA TTAG |

The PSORT algorithm predicts outer membrane (0.887).

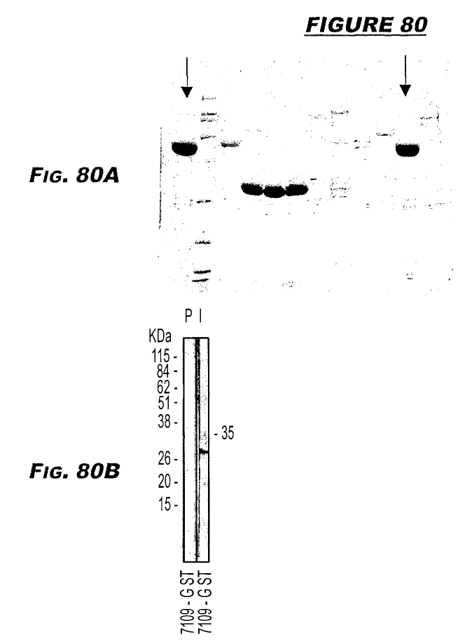

The protein was expressed in E. coli and purified as a GST-fusion product (FIG. 80A). The recombinant protein was used to immunize mice, whose sera were used in a Western blot (FIG. 80B) and for FACS analysis.

These experiments show that cp7109 is a surface-exposed and immunoaccessible protein, and that it is a useful immunogen. These properties are not evident from the sequence alone.

### Example 81

The following C. pneumoniae protein (PID 4377110) was expressed <SEQ ID 161; cp7110>:

| | |
|---|---|
| 1 | MAAIKQILRS MLSQSSLWMV LFSLYSLSGY CYVITDKPED DFHSSSAVKW |
| 51 | DHWGKTTLSR LSNKKASAKA VSGTGATTVG FIKDTWSRTY AVRWNYWGTK |
| 101 | ELPTSSWVKK SKATGISSDG SIIAGIVENE LSQSFAVTWK NNEMYLLPST |
| 151 | WAVQSKAYGI SSDGSVIVGS AKDAWSRTFA VKWTGHEAQV LPVGWAVKSV |
| 201 | ANSVSANGSI IVGSVQDASG ILYAVKWEGN TITHLGTLGG YSAIAKAVSN |
| 251 | NGKVIVGRSE TYYGEVHAFC HKNGVMSDLG TLGGSYSAAK GVSATGKVIV |
| 301 | GMSTTANGKL HAFKYVGGRM IDLGEYSWKE ACANAVSIDG EIIVGVQSE* |

A predicted signal peptide is highlighted.

The cp7110 nucleotide sequence <SEQ ID 162> is:

| | |
|---|---|
| 1 | ATGGCAGCTA TAAAACAAAT TTTACGTTCT ATGCTATCTC AGAGTAGCTT |
| 51 | ATGGATGGTC CTATTTTCAT TATATTCTCT ATCTGGTTAT TGCTATGTAA |
| 101 | TTACAGACAA ACCAGAAGAT GACTTCCATT CTTCATCCGC AGTAAAATGG |
| 151 | GATCATTGGG GAAAGACAAC TCTCTCAAGA TTATCAAATA AAAAAGCCTC |
| 201 | TGCAAAAGCT GTTTCAGGAA CTGGTGCTAC AACTGTCGGC TTTATAAAAG |
| 251 | ACACTTGGTC TCGAACATAC GCAGTAAGAT GGAATTATTG GGGGACCAAA |
| 301 | GAACTCCCTA CCAGCTCATG GGTAAAAAAA TCAAAAGCAA CAGGAATCTC |
| 351 | CTCTGATGGG TCTATAATCG CGGGGATTGT CGAGAATGAG CTTTCTCAAA |
| 401 | GTTTCGCAGT CACATGGAAA AACAATGAAA TGTATTTGCT CCCTTCCACA |
| 451 | TGGGCAGTGC AATCTAAAGC GTATGGAATT TCTTCTGATG GCTCTGTTAT |
| 501 | TGTAGGGAGT GCTAAGGATG CTTGGTCGCG AACTTTCGCT GTGAAGTGGA |
| 551 | CGGGACACGA GGCTCAGGTG TTACCAGTAG GCTGGGCTGT CAAATCTGTA |
| 601 | GCGAATTCTG TATCTGCCAA TGGATCTATA ATTGTAGGGT CTGTACAAGA |
| 651 | CGCCTCTGGA ATTCTTTATG CTGTAAAGTG GGAAGGGAAC ACTATTACAC |
| 701 | ATCTAGGAAC TTTAGGAGGC TATTCTGCCA TTGCAAAAGC TGTATCCAAT |
| 751 | AATGGCAAGG TCATTGTAGG GAGATCCGAA ACATATTATG GAGAGGTCCA |
| 801 | TGCTTTCTGT CATAAGAATG GCGTCATGTC AGACCTCGGC ACCCTCGGAG |
| 851 | GATCTTATTC TGCAGCTAAG GGAGTCTCTG CAACTGGAAA AGTTATTGTC |
| 901 | GGTATGTCCA CAACAGCAAA TGGGAAATTG CATGCCTTTA AATATGTCGG |
| 951 | TGGAAGAATG ATCGACTTAG GAGAGTATAG CTGGAAAGAA GCCTGTGCAA |
| 1001 | ACGCTGTTTC TATTGATGGA GAAATTATTG TTGGAGTCCA ATCAGAATAA |

The PSORT algorithm predicts outer membrane (0.827).

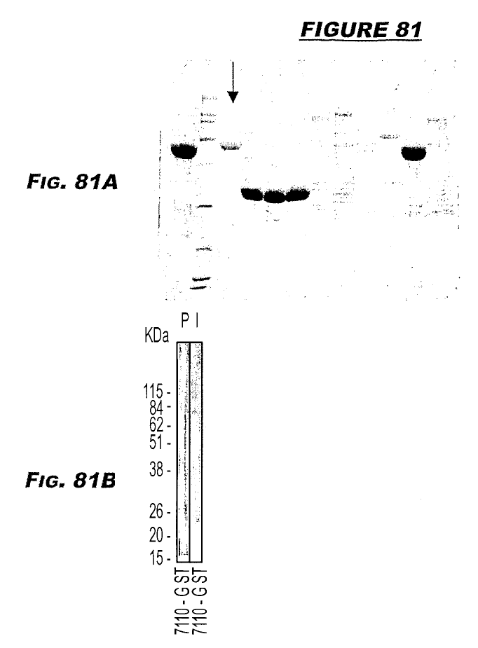

The protein was expressed in E. coli and purified as a GST-fusion product (FIG. 81A). The recombinant protein was used to immunize mice, whose sera were used in a Western blot (FIG. 81B) and for FACS analysis.

These experiments show that cp7110 is a surface-exposed and immunoaccessible protein, and that it is a useful immunogen. These properties are not evident from the sequence alone.

FIG. 191 shows a schematic representation of the structural relationships between of cp7105, cp7106, cp7107, cp7108, cp7109 and cp7110, each of which is identified herein. These six proteins may be grouped in a new family of related outer membrane-associated proteins. These proteins have a repeat structure in common (cf. the pmp family).

### Example 82

The following C. pneumoniae protein (PID 4377127) was expressed <SEQ ID 163; cp7127>:

| | |
|---|---|
| 1 | MVFFRNSLLH LVALSGMLCC SSGVALTIAE KMASLEHSGR GADDYEGMAS |
| 51 | FNANMREYSL QLSKLYEEAR KLRASGTEDE ALWKDLIRRI GEVRGYLREI |
| 101 | EELWAAEIRE KGGNLEDYAL WNHPETTIYN LVTDYGTEDS IYLIPQEIGA |
| 151 | IKIATLSKFV VPKESFEDCL TQILSRLGIG VRQVNSWIKE LYMMRKEGCS |
| 201 | VAGVFSSRKD LEALPETAYI GFVLNSNVDA HTNQHVLKKF INPETTHVDV |
| 251 | IAGRVWIFGS AGEVGELLKI YNFVQSISIR QEYRVIPLTK IDPGEMISIL |
| 301 | NAAFREDLTK DVSEESLGLR VVPLQYQGRS LFLSGTAALV QQALTLIREL |
| 351 | EEGIENPTDK TVFWYNVKHS DPQELAALLS QVHDVFSGEN KASVGAADGC |
| 401 | GSQLNASIQI DTTVSSSAKD GSVKYGNFIA DSKTGTLIMV VEKEVLPRIQ |
| 451 | MLLKKLDVPK KMVRIEVLLF ERKLAHEQKS GLNLLRLGEE VCKKGCSPSV |
| 501 | SWAGGTGILE FLFKGSTGSS IVPGYDLAYQ FLMAQEDVRI NASPSVVTMN |
| 551 | QTPARIAVVD EMSIAVSSDK DKAQYNRAQY GIMIKMLPVI NVGEEDGKSY |
| 601 | ITLETDITFD TTGKNHDDRP DVTRRNITNK VRIADGETVI IGGLRCKQMS |
| 651 | DSHDGIPFLG DIPGIGKLFG MSSTSDSLTE MFVFITPKIL ENPVEQQERK |
| 701 | EEALLSSRPG EREEYYQALA ASEAAARAAH KKLEMFPASG VSLSQVERQE |
| 751 | YDGC* |

A predicted signal peptide is highlighted.

The cp7127 nucleotide sequence <SEQ ID 164> is:

| | |
|---|---|
| 1 | ATGGTTTTTT TCCGTAATTC TTTACTGCAT TTAGTTGCCC TATCCGGAAT |
| 51 | GCTCTGTTGT TCTTCTGGAG TGGCTTTAAC GATAGCCGAG AAGATGGCTT |
| 101 | CTTTAGAGCA CTCGGGGAGA GGAGCAGACG ATTATGAGGG GATGGCTTCG |
| 151 | TTTAATGCCA ATATGAGGGA GTATAGCCTT CAGCTGAGCA AGTTGTATGA |
| 201 | GGAAGCACGA AAGCTACGCG CTTCTGGAAC TGAGGATGAA GCTCTGTGGA |
| 251 | AGGACTTAAT TCGACGGATT GGTGAGGTGC GAGGCTATCT TCGAGAGATC |
| 301 | GAGGAGCTTT GGGCTGCAGA AATTCGTGAG AAAGGGGGCA ATCTCGAGGA |
| 351 | CTACGCCCTC TGGAATCACC CAGAGACTAC GATTTACAAT CTTGTTACCG |
| 401 | ATTACGGAAC CGAAGACTCT ATTTATTTGA TTCCTCAAGA AATCGGAGCG |
| 451 | ATTAAAATCG CAACCTTATC GAAATTTGTA GTTCCTAAAG AGTCTTTCGA |
| 501 | AGACTGTCTC ACTCAGATCC TATCTCGCTT AGGTATTGGC GTGCGTCAGG |
| 551 | TCAATTCTTG GATTAAGGAA CTTTATATGA TGCGTAAGGA GGGCTGCAGT |
| 601 | GTTGCTGGAG TTTTTTCCTC CAGAAAAGAT TTAGAGGCGC TCCCAGAAAC |
| 651 | AGCCTATATT GGTTTTGTAT TGAATTCGAA CGTAGATGCG CATACCAATC |
| 701 | AACATGTCTT AAAAAAGTTC ATTAACCCTG AAACAACGCA TGTAGATGTG |
| 751 | ATTGCAGGAC GTGTGTGGAT TTTTGGTTCT GCGGGGGAAG TCGGCGAGCT |
| 801 | TCTGAAGATT TATAATTTTG TGCAGTCGGA GAGCATACGT CAAGAGTATC |
| 851 | GGGTGATTCC CTTAACTAAG ATCGATCCAG GGGAGATGAT TTCCATTCTC |
| 901 | AACGCAGCAT TTCGTGAGGA TCTGACTAAA GATGTTAGTG AAGAATCTTT |
| 951 | AGGCCTTCGT GTAGTTCCTT TACAGTATCA AGGGCGTTCG TTGTTTTTAA |
| 1001 | GTGGAACCGC GGCGTTAGTG CAGCAAGCGC TGACTCTCAT TCGAGAGCTT |
| 1051 | GAAGAAGGGA TTGAGAACCC TACGGATAAA ACAGTATTTT GGTATAACGT |
| 1101 | CAAGCACTCC GATCCCCAAG AGTTGGCGGC ATTGCTTTCC CAAGTCCATG |
| 1151 | ATGTCTTCTC TGGCGAGAAT AAGGCGAGTG TCGGAGCTGC AGATGGATGT |
| 1201 | GGGTCGCAAT TAAATGCCTC GATCCAAATT GATACTACAG TAAGTTCTTC |
| 1251 | TGCGAAAGAT GGCTCAGTGA AGTACGGAAA CTTCATCGCG GATTCTAAGA |
| 1301 | CAGGAACTCT GATTATGGTG GTTGAGAAAG AAGTTCTTCC ACGTATTCAG |
| 1351 | ATGCTACTTA AGAAACTAGA TGTCCCTAAA AAGATGGTCC GTATCGAGGT |
| 1401 | GCTGTTATTT GAAAGAAAAT TGGCACATGA GCAGAAATCT GGGTTAAATC |
| 1451 | TTCTACGTCT TGGTGAGGAA GTTTGTAAAA AAGGGTGCAG TCCTTCTGTG |
| 1501 | TCTTGGGCCG GGGGTACTGG CATACTAGAA TTTTTATTTA AAGGAAGTAC |
| 1551 | GGGATCTTCG ATAGTTCCTG GTTATGATCT CGCCTATCAA TTTTTAATGG |
| 1601 | CTCAAGAGGA CGTTCGGATT AATGCGAGTC CTTCTGTAGT TACTATGAAC |
| 1651 | CAAACCCCAG CACGGATTGC TGTTGTTGAT GAAATGTCAA TAGCGGTGTC |
| 1701 | TTCAGATAAA GATAAAGCGC AATACAATCG TGCGCAGTAC GGTATCATGA |
| 1751 | TAAAAATGCT CCCCGTAATT AATGTGGGAG AGGAAGACGG AAAAAGTTAC |
| 1801 | ATTACTTTAG AGACAGACAT CACCTTTGAT ACTACGGGAA AAAATCATGA |
| 1851 | TGATCGTCCT GATGTTACAA GGCGTAATAT TACTAATAAG GTGCGCATTG |
| 1901 | CTGACGGAGA GACTGTGATT ATTGGAGGTT TGCGTTGCAA ACAGATGTCA |
| 1951 | GATTCTCATG ATGGCATTCC TTTCCTTGGA GACATTCCTG GTATAGGGAA |
| 2001 | GTTATTTGGA ATGAGTTCCA CATCAGACAG TCTCACGGAG ATGTTTGTAT |
| 2051 | TTATCACTCC GAAGATCCTA GAAAATCCTG TAGAGCAACA AGAACGTAAA |
| 2101 | GAAGAAGCTT TACTCTCTTC GCGCCCTGGA GAGAGAGAAG AATACTATCA |
| 2151 | GGCTTTAGCA GCTAGTGAGG CTGCAGCACG AGCAGCTCAT AAAAAATTAG |
| 2201 | AGATGTTCCC GGCATCAGGA GTATCTTTAT CTCAGGTAGA GAGGCAAGAA |
| 2251 | TACGATGGCT GCTAG |

The PSORT algorithm predicts periplasmic (0.920).

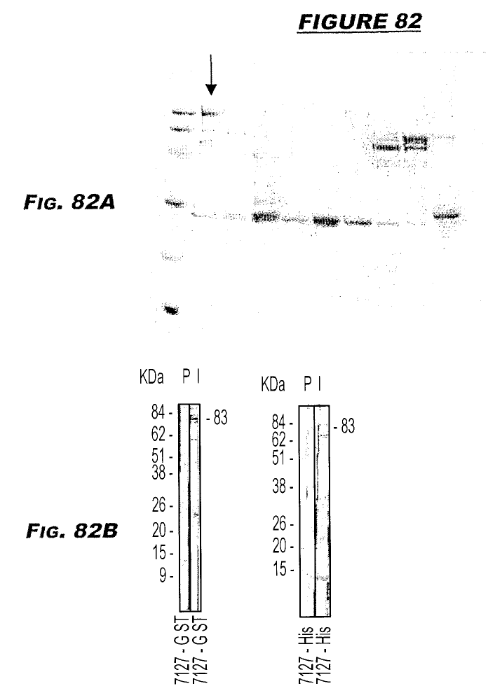

The protein was expressed in E. coli and purified as a GST-fusion product (FIG. 82A) and also in his-tagged form. The recombinant proteins were used to immunize mice, whose sera were used in a Western blot (FIG. 82B) and for FACS analysis.

These experiments show that cp7127 is a surface-exposed and immunoaccessible protein, and that it is a useful immunogen. These properties are not evident from the sequence alone.

### Example 83

The following C. pneumoniae protein (PID 4377133) was expressed <SEQ ID 165; cp7133>:

| | |
|---|---|
| 1 | MQPFIFTLLC LTSLVSLVAF DAANARKRCA CAQTIERGEN FFSIKRSACA |
| 51 | EIEYQEKSRH ASAIERISKD KGKVTPKQIA KVATKKKQRY RLLQVPFSRP |
| 101 | PNNSRYNLYA LLSEPPECYS DTASWYAIFI RLLRRAYVDT GNVPPGSEYA |
| 151 | IANALISNKQ EILERGAQLG PDVIETLTLP EEQAEIFYKM LKGSSNSQSL |
| 201 | LNFLHYEEKS LGHCKLNLIF MDPLLLEAVL DHPDAYRETS LLRDGIWEAV |
| 251 | KRQEHAIQEH GQAAALELFK TRTDFRLELR DKMQLLLSRY DLLPLLNKKM |
| 301 | FDYTLGSAGD YLFLVDPDTK AISRCRCPSK SIKL |

A predicted signal peptide is highlighted.

The cp7133 nucleotide sequence <SEQ ID 166> is:

| | |
|---|---|
| 1 | ATGCAACCTT TTATCTTTAC TTTACTGTGC TTGACATCTT TGGTTTCTTT |
| 51 | AGTCGCCTTT GATGCTGCGA ATGCTCGTAA ACGTTGTGCC TGTGCTCAAA |
| 101 | CTATAGAACG TGGAGAGAAC TTCTTTTCCA TAAAACGCTC TGCTTGTGCT |
| 151 | GAAATCGAAT ATCAAGAAAA ATCTCGCCAC GCCTCAGCAA TTGAAAGAAT |
| 201 | CTCAAAAGAT AAAGGCAAAG TCACTCCAAA GCAGATTGCG AAAGTAGCTA |
| 251 | CTAAGAAAAA GCAAAGATAC CGTTTATTGC AGGTTCCTTT TTCAAGGCCT |
| 301 | CCGAATAACT CAAGGTATAA CCTCTATGCT TTGCTTAGTG AACCTCCCGA |
| 351 | ATGCTATAGC GATACAGCAT CATGGTATGC TATTTTTATT CGGTTACTTC |
| 401 | GACGTGCTTA TGTAGACACG GGAAATGTAC CTCCTGGATC TGAGTATGCC |
| 451 | ATCGCTAATG CTTTGATAAG TAACAAACAA GAGATTTTAG AGAGGGGAGC |
| 501 | GCAGCTTGGA CCCGATGTTA TTGAAACTCT AACATTGCCT GAGGAACAAG |
| 551 | CCGAGATTTT TTATAAAATG CTCAAAGGGT CGTCAAACTC TCAGTCGCTA |
| 601 | CTGAATTTTC TGCATTATGA AGAGAAAAGC TTAGGCCACT GTAAGCTAAA |
| 651 | TCTGATCTTC ATGGATCCCC TACTGTTAGA AGCTGTTCTA GATCATCCCG |
| 701 | ATGCTTATAG GGAAACGTCG CTCCTGCGCG ATGGCATTTG GGAAGCGGTG |
| 751 | AAGCGTCAAG AACATGCCAT CCAAGAACAT GGCCAGGCAG CTGCTTTGGA |
| 801 | GCTTTTTAAA ACACGCACCG ACTTCCGCCT GGAGCTGCGA GATAAGATGC |
| 851 | AGTTACTTCT AAGTCGATAC GATTTGCTCC CCTTATTAAA TAAAAAAATG |
| 901 | TTCGACTACA CCTTAGGAAG TGCCGGAGAT TACTTATTTT TGGTAGACCC |
| 951 | AGATACTAAG GCAATTTCTC GATGTCGCTG CCCTTCAAAG AGTATTAAAT |
| 1001 | TATAA |

The PSORT algorithm predicts outer membrane (0.92).

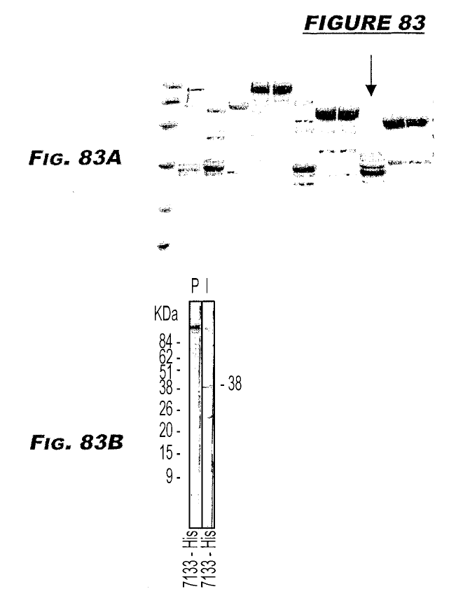

The protein was expressed in E. coli and purified as a GST-fusion product (FIG. 83A) and also in his-tagged form. The recombinant proteins were used to immunize mice, whose sera were used in a Western blot (FIG. 83B) and for FACS analysis.

These experiments show that cp7133 is a surface-exposed and immunoaccessible protein, and that it is a useful immunogen. These properties are not evident from the sequence alone.

### Example 84

The following C. pneumoniae protein (PID 4377222) was expressed <SEQ ID 167; cp7222>:

| | |
|---|---|
| 1 | MNRRDMVITA VVVNAILLVA LFVTSKRIGV KDYDEGFRNF ASSKVTQAVV |
| 51 | SEEKVIEKPV VAEVPSRPIA KETLAAQFIE SKPVIVTTPP VPVVSETPEV |
| 101 | PTVAVPPQPV RETVKEEQAP YATVVVKKGD FLERIARANH TTVAKLMQIN |
| 151 | DLTTTQLKIG QVIKVPTSQD VSNEKTPQTQ TANPENYYIV QEGDSPWTIA |
| 201 | LRNHIRLDDL LKMNDLDEYK ARRLKPGDQL RIR* |

A predicted signal peptide is highlighted.

The cp7222 nucleotide sequence <SEQ ID 168> is:

| | |
|---|---|
| 1 | ATGAATCGTA GAGACATGGT AATAACAGCT GTCGTAGTGA ATGCTATATT |
| 51 | GCTTGTGGCT CTTTTCGTCA CATCAAAGCG TATTGGCGTC AAGGACTATG |
| 101 | ACGAGGGATT CCGTAATTTT GCTTCTAGCA AGGTTACACA AGCAGTAGTT |
| 151 | TCAGAAGAAA AAGTCATAGA AAAGCCTGTA GTCGCAGAAG TGCCTAGCCG |
| 201 | TCCTATCGCT AAAGAGACTC TAGCTGCACA GTTTATTGAA AGTAAGCCGG |
| 251 | TTATTGTAAC CACACCACCC GTGCCTGTTG TTAGCGAAAC CCCAGAAGTG |
| 301 | CCTACTGTGG CAGTTCCGCC TCAGCCTGTT CGTGAGACAG TAAAAGAGGA |
| 351 | ACAAGCTCCT TATGCTACTG TTGTAGTGAA AAAAGGAGAT TTTCTCGAAC |
| 401 | GCATTGCGAG AGCAAATCAT ACTACCGTTG CAAAATTGAT GCAGATCAAT |
| 451 | GATCTTACCA CCACCCAACT TAAAATTGGT CAGGTCATCA AAGTCCCTAC |
| 501 | GTCTCAAGAT GTCAGCAACG AAAAAACTCC TCAAACACAG ACCGCAAACC |
| 551 | CTGAAAATTA TTATATCGTC CAAGAAGGGG ATAGCCCGTG GACAATAGCA |
| 601 | TTGCGTAACC ATATTCGATT GGATGATTTG CTAAAAATGA ATGATCTCGA |
| 651 | TGAATATAAA GCCCGGCGCC TTAAGCCTGG AGATCAGTTG CGCATACGTT |
| 701 | GA |

The PSORT algorithm predicts periplasmic (0.935).

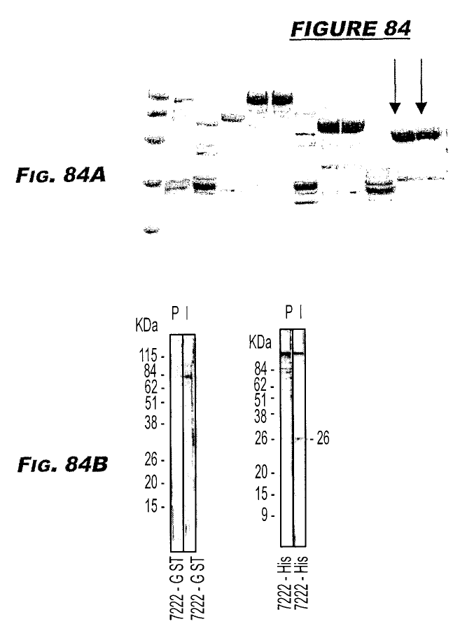

The protein was expressed in E. coli and purified as a GST-fusion product (FIG. 84A) and also in his-tagged form. The recombinant proteins were used to immunize mice, whose sera were used in a Western blot (FIG. 84B) and for FACS analysis.

These experiments show that cp7222 is a surface-exposed and immunoaccessible protein, and that it is a useful immunogen. These properties are not evident from the sequence alone.

### Example 85

The following C. pneumoniae protein (PID 4377225) was expressed <SEQ ID 169; cp7225>:

| | |
|---|---|
| 1 | MKGTPQYHFI GIGGIGMSAL AHILLDRGYE VSGSDLYESY TIESLKAKGA |
| 51 | RCFSGHDSSH VPHDAVVVYS SSIAPDNVEY LTAIQRSSRL LHRAELLSQL |
| 101 | MEGYESILVS GSHGKTGTSS LIRAIFQEAQ KDPSYAIGGL AANCLNGYSG |
| 151 | SSKIFVAEAD ESDGSLKHYT PRAVVITNID NEHLNNYAGN LDNLVQVIQD |
| 201 | FSRKVTDLNK VFYNGDCPIL KGNVQGISYG YSPECQLHIV SYNQKAWQSH |
| 251 | FSFTFLGQEY QDIELNLPGQ HNAANAAAAC GVALTFGIDI NIIRKALKKF |
| 301 | SGVHRRLERK NISESFLFLE DYAHHPVEVA HTLRSVRDAV GLRRVIAIFQ |
| 351 | PHRFSRLEEC LQTFPKAFQE ADEVILTDVY SAGESPRESI ILSDLAEQIR |
| 401 | KSSYVHCCYV PHGDIVDYLR NYIRIHDVCV SLGAGNIYTI GEALKDFNPK |
| 451 | KLSIGLVCGG KSCEHDISLL SAQHVSKYIS PEFYDVSYFI INRQGLWRTG |
| 501 | KDFPHLIEET QGDSPLSSEI ASALAKVDCL FPVLHGPFGE DGTIQGFFEI |
| 551 | LGKPYAGPSL SLAATAMDKL LTKRIASAVG VPVVPYQPLN LCFWKRNPEL |
| 601 | CIQNLIETFS FPMIVKTAHL GSSIGIFLVR DKEELQEKIS EAFLYDTDVF |
| 651 | VEESRLGSRE IEVSCIGHSS SWYCMAGPNE RCGASGFIDY QEKYGFDGID |
| 701 | CAKISFDLQL SQESLDCVRE LAERVYRAMQ GKGSARIDFF LDEEGNYWLS |
| 751 | EVNPIPGMTA ASPFLQAFVH AGWTQEQIVD HFIIDALHKF DKQQTIEQAF |
| 801 | TKEQDLVKR* |

The cp7225 nucleotide sequence <SEQ ID 170> is:

| | |
|---|---|
| 1 | ATGAAGGGAA CTCCTCAGTA TCATTTTATC GGTATCGGTG GTATAGGAAT |
| 51 | GAGCGCTTTA GCTCATATTT TGCTTGATCG TGGCTATGAG GTCTCTGGAA |
| 101 | GCGACTTATA TGAAAGCTAT ACGATCGAAA GCCTGAAAGC TAAAGGTGCG |
| 151 | AGGTGTTTCT CAGGCCATGA TTCCTCCCAT GTTCCTCATG ATGCCGTCGT |
| 201 | TGTTTATAGC TCAAGTATAG CCCCTGATAA TGTAGAGTAT CTTACCGCTA |
| 251 | TTCAAAGATC ATCACGTCTT CTTCATAGAG CAGAGCTCTT GAGTCAGCTT |
| 301 | ATGGAGGGTT ATGAAAGCAT TCTGGTTTCA GGAAGCCATG GGAAGACAGG |
| 351 | GACCTCATCT CTAATTCGAG CGATTTTCCA GGAAGCTCAG AAAGATCCCT |
| 401 | CCTATGCTAT TGGAGGACTC GCTGCAAACT GCCTGAATGG GTATTCTGGA |
| 451 | TCATCGAAAA TCTTCGTTGC CGAAGCCGAT GAAAGTGATG GGTCTTTAAA |
| 501 | GCACTACACT CCCCGTGCAG TAGTCATTAC AAATATAGAT AATGAACATT |
| 551 | TGAATAATTA CGCTGGGAAT CTTGATAACC TGGTTCAGGT AATCCAGGAC |
| 601 | TTCTCTAGAA AAGTAACAGA TCTCAATAAG GTATTCTATA ACGGGGATTG |
| 651 | TCCTATTTTG AAAGGAAATG TCCAAGGGAT TTCTTATGGA TATTCACCAG |
| 701 | AATGTCAATT GCATATCGTT TCCTATAATC AAAAGGCATG GCAATCTCAC |
| 751 | TTTTCCTTTA CCTTTTTAGG CCAGGAGTAT CAAGACATTG AGCTCAATCT |
| 801 | CCCTGGACAA CATAACGCTG CAAATGCAGC AGCAGCCTGT GGAGTTGCTC |
| 851 | TTACCTTTGG CATAGACATA AACATCATTC GAAAAGCTCT CAAAAAATTC |
| 901 | TCGGGAGTTC ATCGACGTCT AGAAAGAAAA AATATATCCG AAAGCTTTCT |
| 951 | TTTCTTAGAA GATTATGCTC ATCATCCTGT AGAGGTTGCA CATACCCTGC |
| 1001 | GCTCTGTGCG TGATGCTGTG GGTTTGCGAA GAGTCATCGC AATTTTTCAA |
| 1051 | CCACATCGAT TCTCTCGTTT AGAAGAGTGC TTACAAACCT TCCCCAAAGC |
| 1101 | TTTCCAAGAA GCTGATGAAG TCATACTTAC AGATGTCTAT AGTGCCGGAG |
| 1151 | AAAGTCCTAG AGAGTCTATC ATTCTTTCCG ACCTTGCGGA ACAGATTCGT |
| 1201 | AAGTCTTCTT ATGTCCATTG TTGTTATGTT CCCCATGGAG ACATCGTAGA |
| 1251 | TTATCTACGA AACTACATTC GCATTCATGA TGTCTGTGTT TCTCTAGGAG |
| 1301 | CTGGAAATAT CTATACTATT GGAGAGGCTT TAAAAGACTT TAACCCTAAA |
| 1351 | AAATTATCCA TAGGACTCGT CTGTGGAGGG AAATCTTGCG AACACGATAT |
| 1401 | TTCTCTACTT TCTGCTCAAC ATGTCTCTAA ATATATTTCT CCTGAATTCT |
| 1451 | ATGATGTGAG TTACTTCATC ATAAATCGTC AGGGCTTATG GAGAACAGGA |
| 1501 | AAGGATTTTC CTCATCTTAT TGAAGAGACT CAAGGGGATT CGCCACTTTC |
| 1551 | TTCTGAAATC GCTTCAGCTT TAGCAAAAGT CGACTGTTTG TTTCCCGTGC |
| 1601 | TCCATGGCCC ATTTGGAGAG GATGGTACGA TCCAGGGATT TTTTGAAATC |
| 1651 | TTAGGAAAAC CTTATGCCGG ACCCTCACTA TCTTTAGCAG CAACTGCAAT |
| 1701 | GGATAAGCTG TTAACAAAAC GAATTGCATC AGCAGTGGGT GTTCCTGTAG |
| 1751 | TCCCTTACCA ACCTTTAAAT CTCTGTTTCT GGAAACGCAA TCCAGAACTA |
| 1801 | TGTATTCAGA ATCTTATAGA GACATTTTCT TTCCCTATGA TTGTAAAAAC |
| 1851 | TGCACATTTG GGATCTAGTA TTGGGATATT TTTAGTCCGT GATAAAGAGG |
| 1901 | AATTACAAGA AAAGATCTCA GAAGCATTTC TATATGACAC GGATGTGTTT |
| 1951 | GTGGAGGAAA GTCGCTTAGG GTCTCGTGAA ATCGAAGTGT CCTGTATCGG |
| 2001 | CCATTCTTCT AGCTGGTATT GTATGGCAGG GCCTAATGAA CGCTGTGGTG |
| 2051 | CTAGTGGGTT TATTGATTAT CAAGAGAAAT ATGGATTTGA TGGCATAGAT |
| 2101 | TGCGCAAAGA TCTCTTTTGA TTTACAGCTC TCACAAGAAT CTTTAGATTG |
| 2151 | TGTTAGAGAA CTTGCAGAGC GTGTCTACCG AGCAATGCAA GGAAAAGGTT |
| 2201 | CAGCTCGAAT AGATTTTTTC TTGGATGAAG AGGGGAATTA TTGGTTGTCA |
| 2251 | GAGGTCAATC CTATTCCAGG AATGACAGCA GCTAGCCCAT TTTTACAAGC |
| 2301 | TTTTGTTCAC GCAGGATGGA CGCAAGAACA AATTGTAGAT CACTTTATTA |
| 2351 | TAGATGCTCT ACATAAGTTT GATAAGCAGC AGACTATCGA ACAGGCATTC |
| 2401 | ACTAAAGAAC AAGATTTAGT TAAAAGATAA |

The PSORT algorithm predicts inner membrane (0.16).

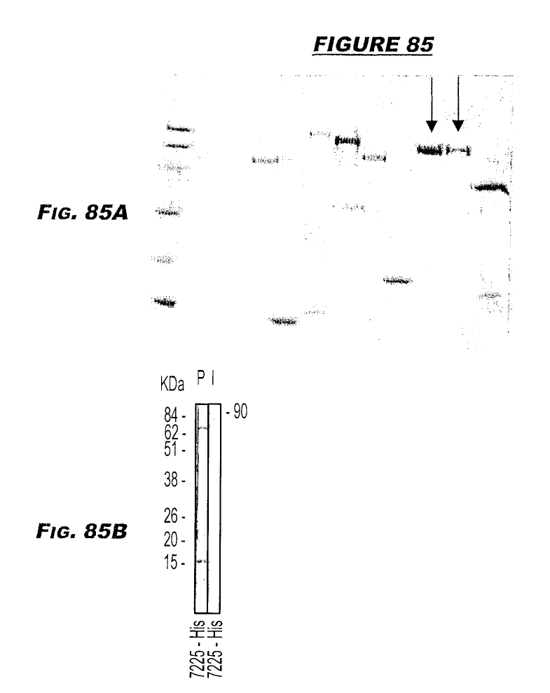

The protein was expressed in E. coli and purified as a his-tag product (FIG. 85A). The recombinant protein was used to immunize mice, whose sera were used in a Western blot (FIG. 85B) and for FACS analysis.

These experiments show that cp7225 is a surface-exposed and immunoaccessible protein, and that it is a useful immunogen. These properties are not evident from the sequence alone.

### Example 86

The following C. pneumoniae protein (PID 4377248) was expressed <SEQ ID 171; cp7248>:

| | |
|---|---|
| 1 | MKFWLQGCAF VGCLLLTLPC CAARRRASGE NLQQTRPIAA ANLQWESYAE |
| 51 | ALEHSKQDHK PICLFFTGSD WCMWCIKMQD QILQSSEFKH FAGVHLHMVE |
| 101 | VDFPQKNHQP EEQRQKNQEL KAQYKVTGFP ELVFIDAEGK QLARMGFEPG |
| 151 | GGAAYVSKVK SALKLR* |

A predicted signal peptide is highlighted.

The cp7248 nucleotide sequence <SEQ ID 172> is:

| | |
|---|---|
| 1 | ATGAAATTTT GGTTGCAAGG ATGTGCTTTT GTCGGTTGTC TGCTATTGAC |
| 51 | TTTACCTTGT TGTGCTGCAC GAAGACGTGC TTCTGGAGAA AATTTGCAAC |
| 101 | AAACTCGTCC TATAGCAGCT GCAAATCTAC AATGGGAGAG CTATGCAGAA |
| 151 | GCTCTTGAAC ATTCTAAACA AGATCACAAA CCTATTTGTC TTTTCTTTAC |
| 201 | AGGATCAGAC TGGTGTATGT GGTGCATAAA AATGCAAGAC CAGATTTTGC |
| 251 | AAAGCTCTGA GTTTAAGCAT TTTGCGGGTG TGCATCTGCA TATGGTTGAA |
| 301 | GTTGATTTCC CCCAAAAGAA TCATCAACCT GAAGAGCAGC GCCAAAAAAA |
| 351 | TCAAGAACTG AAAGCTCAAT ATAAAGTTAC AGGATTCCCC GAACTGGTCT |
| 401 | TCATAGATGC AGAAGGAAAA CAGCTTGCTC GCATGGGATT TGAGCCTGGT |
| 451 | GGTGGAGCTG CTTACGTAAG CAAGGTGAAG TCTGCTCTTA AACTACGTTA |
| 501 | A |

The PSORT algorithm predicts periplasmic (0.932).

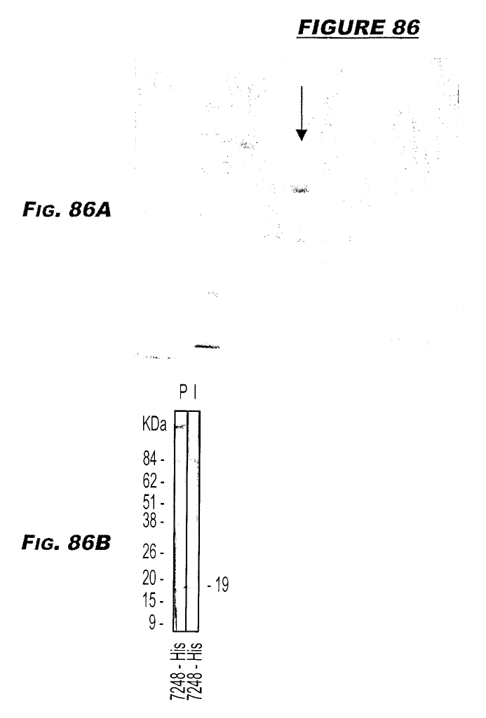

The protein was expressed in E. coli and purified as a GST-fusion product (FIG. 86A) and also in his-tagged form. The recombinant proteins were used to immunize mice, whose sera were used in a Western blot (FIG. 86B) and for FACS analysis.

The cp7248 protein was also identified in the 2D-PAGE experiment.

These experiments show that cp7248 is a surface-exposed and immunoaccessible protein, and that it is a useful immunogen. These properties are not evident from the sequence alone.

### Example 87

The following C. pneumoniae protein (PID 4377249) was expressed <SEQ ID 173; cp7249>:

| | |
|---|---|
| 1 | MIPSPTPINF RDDTILETDP KPSLIMFSSK KTEIASERRK AHPTLFKVLG |
| 51 | TIWNIVKFII SIILFLPLAL LWVLKKTCQF FILPSSIISQ SMSKTAVAIR |
| 101 | RMTFLSHIKQ LLSLKEISAA DRVVIQYDDL VVDSLAIKIP HALPHRWILY |
| 151 | SQGNSGLMEN LFDRGDSSLH QLAKATGSNL LVFNYPGIMS SKGEAKRENL |
| 201 | VKSYQACVRY LRDEETGPKA NQIIAFGYSL GTSVQAAALD REVTDGSDGT |
| 251 | SWIVVKDRGP RSLADVANQI CKPIASAIIK LVGWNIDSVK PSERLRCPEI |
| 301 | FIYNSNHDQE LISDGLFERE NCVATPFLEL PEVKTSGTKI PIPERDLLHL |
| 351 | NPLSPNVVDR LAAVISNYLD SENRKSQQPD * |

The cp7249 nucleotide sequence <SEQ ID 174> is:

| | |
|---|---|
| 1 | ATGATCCCAT CCCCTACCCC AATAAACTTT CGTGATGATA CGATTCTAGA |
| 51 | GACGGATCCA AAGCCGTCTT TAATCATGTT CTCTTCAAAA AAAACAGAGA |
| 101 | TAGCTTCTGA AAGACGGAAG GCCCATCCCA CCTTATTTAA AGTTCTAGGA |
| 151 | ACGATTTGGA ATATTGTGAA GTTTATTATC TCAATCATTC TGTTCCTTCC |
| 201 | CTTAGCGTTA TTGTGGGTAC TCAAGAAAAC CTGTCAGTTT TTCATTCTCC |
| 251 | CATCTTCTAT CATATCTCAG AGCATGTCAA AAACAGCTGT GGCAATTCGG |
| 301 | CGAATGACCT TTCTGTCCCA TATTAAACAA CTCCTAAGCC TTAAGGAAAT |
| 351 | CTCAGCTGCC GATCGTGTGG TTATACAATA TGACGATTTG GTGGTTGATA |
| 401 | GCTTAGCTAT AAAGATACCT CATGCTCTTC CCCACAGGTG GATTCTTTAT |
| 451 | TCTCAAGGAA ACTCTGGATT GATGGAAAAC CTGTTCGATC GGGGCGATTC |
| 501 | CTCTCTACAC CAGCTAGCCA AAGCAACCGG CTCGAATCTT CTTGTGTTCA |
| 551 | ACTATCCTGG AATTATGTCC AGCAAAGGAG AAGCGAAACG AGAAAATCTG |
| 601 | GTTAAATCGT ATCAGGCATG CGTACGCTAC CTACGAGATG AAGAGACAGG |
| 651 | TCCTAAAGCC AATCAAATCA TAGCTTTCGG ATACTCTTTG GGAACTAGTG |
| 701 | TCCAAGCTGC TGCTCTAGAT CGTGAGGTCA CTGATGGCAG TGATGGAACT |
| 751 | TCATGGATTG TTGTAAAAGA TCGGGGCCCT CGCTCTCTAG CAGATGTCGC |
| 801 | GAATCAAATT TGTAAGCCCA TAGCTTCCGC GATTATAAAA CTCGTTGGTT |
| 851 | GGAACATAGA CTCTGTGAAA CCTAGCGAAA GATTGCGTTG TCCCGAAATT |
| 901 | TTCATTTACA ACTCTAATCA TGATCAAGAA CTCATTAGCG ACGGCCTCTT |
| 951 | CGAAAGAGAA AATTGCGTAG CAACACCTTT TCTAGAGCTT CCTGAAGTAA |
| 1001 | AAACCTCGGG GACTAAAATT CCTATACCCG AAAGGGATCT TCTCCATCTA |
| 1051 | AATCCTCTCA GTCCAAATGT AGTAGACAGA TTAGCAGCAG TGATCTCTAA |
| 1101 | TTATTTAGAT TCTGAAAACA GAAAGTCTCA GCAACCTGAT TAA |

The PSORT algorithm predicts inner membrane (0.571).

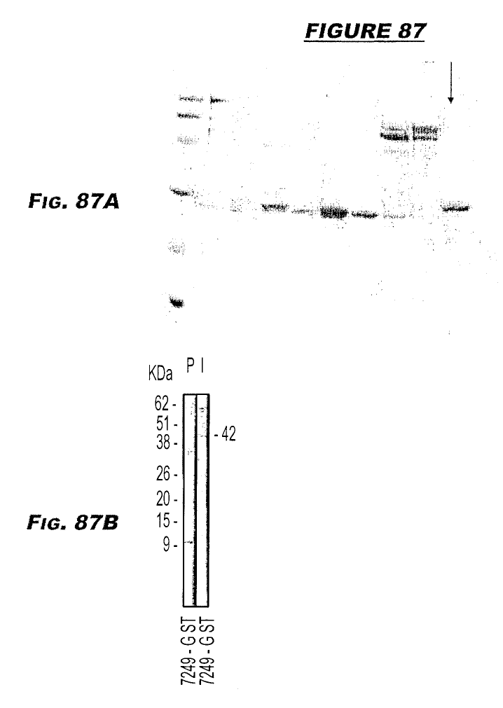

The protein was expressed in E. coli and purified as a GST-fusion product (FIG. 87A). The recombinant protein was used to immunize mice, whose sera were used in a Western blot (FIG. 87B) and for FACS analysis.

These experiments show that cp7249 is a surface-exposed and immunoaccessible protein, and that it is a useful immunogen. These properties are not evident from the sequence alone.

### Example 88

The following C. pneumoniae protein (PID 4377261) was expressed <SEQ ID 175; cp7261>:

| | |
|---|---|
| 1 | MLPISILLFY VILGCLSAYI ADKKKRNVIG WFFAGAFFGF IGLVVLLLLP |
| 51 | SRRNALEKPQ NDPFDNSDLF DDLKKSLAGN DEIPSSGDLQ EIVIDTEKWF |
| 101 | YLNKDRENVG PISFEELVVL LKGKTYPEEI WVWKKGMKDW QRVKDVPSLQ |
| 151 | QALKEASK* |

The cp7261 nucleotide sequence <SEQ ID 176> is:

| | |
|---|---|
| 1 | ATGCTCCCTA TTTCGATTTT ATTATTTTAT GTGATTCTAG GTTGTCTATC |
| 51 | TGCCTACATA GCAGATAAGA AAAAACGAAA TGTTATTGGC TGGTTTTTTG |
| 101 | CAGGAGCATT TTTTGGATTT ATTGGTCTAG TTGTCCTTCT TCTTCTTCCT |
| 151 | TCTCGTCGAA ACGCTTTAGA AAAGCCACAA AACGATCCTT TTGATAACTC |
| 201 | CGATCTTTTT GATGATTTGA AAAAAAGTTT AGCAGGTAAT GACGAGATAC |
| 251 | CCTCATCGGG AGATCTTCAA GAAATCGTTA TCGATACAGA GAAGTGGTTT |
| 301 | TATTTAAATA AAGATAGAGA AAACGTAGGT CCGATATCTT TTGAGGAGTT |
| 351 | GGTCGTACTT TTAAAGGGAA AAACGTATCC AGAAGAAATT TGGGTATGGA |
| 401 | AAAAGGGAAT GAAAGATTGG CAACGAGTGA AGGATGTTCC ATCACTACAA |
| 451 | CAGGCTTTGA AAGAAGCATC AAAATAA |

The PSORT algorithm predicts inner membrane (0.848).

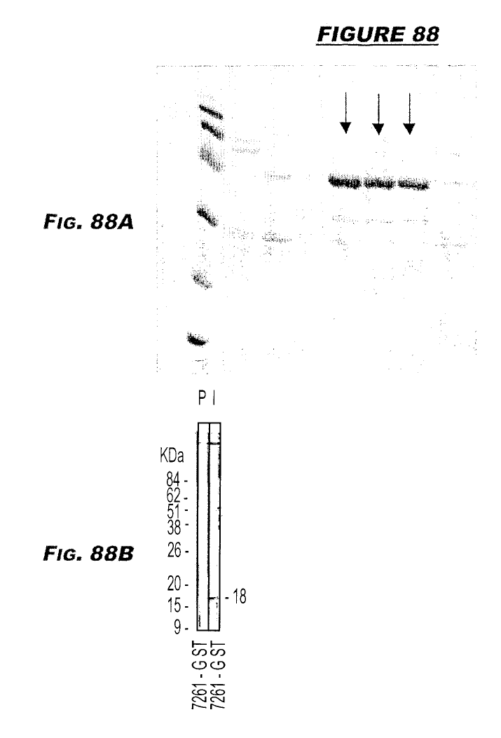

The protein was expressed in E. coli and purified as a GST-fusion product (FIG. 88A). The recombinant protein was used to immunize mice, whose sera were used in a Western blot (FIG. 88B) and for FACS analysis.

These experiments show that cp7261 is a surface-exposed and immunoaccessible protein, and that it is a useful immunogen. These properties are not evident from the sequence alone.

### Example 89

The following C. pneumoniae protein (PID 4377305) was expressed <SEQ ID 177; cp7305>:

| | |
|---|---|
| 1 | MEVYSFHPAV RTSFQHRVMA ALDAWFFLGG HRLKVVSLDS CNSGWAYQEL |
| 51 | VSISTTEKVL KLLSYLLVPI VIIALLIRCL LHSNFRIDVE KERWLKIREL |
| 101 | GIDIESCKLP SSYVNQVSSF IWFEKDKSKR PRIDVDYHTL HSKDWVVFPI |
| 151 | VFQKIPKTSR FSYWFSQKET RKRDYVRNML DHVIGYLTSE GGEWLQYISK |
| 201 | TSYQSATSLD PERVLQYCLT DNQELQGEVQ RLLNEESATK SSGDKEVLLS |
| 251 | HVSDIICQCW WPKFLEVIQS PAFIEELVEE VSGKLNLDFL CLEKANTLDQ |
| 301 | ELRNSLLRAV VHHGSEGVDI KKVGAGLIIY TEAIQLQIPF SRS* |

The cp7305 nucleotide sequence <SEQ ID 178> is:

| | |
|---|---|
| 1 | ATGGAAGTTT ATAGTTTTCA CCCTGCGGTA AGGACTTCGT TTCAGCACCG |
| 51 | TGTAATGGCA GCACTAGATG CTTGGTTTTT TCTAGGAGGG CACCGTTTAA |
| 101 | AAGTAGTTTC TCTAGATAGT TGTAACTCAG GTTGGGCGTA TCAAGAACTT |
| 151 | GTGTCTATTT CAACGACAGA AAAAGTCTTG AAACTACTCT CTTACCTACT |
| 201 | CGTACCGATT GTCATAATAG CTCTGTTAAT TCGTTGTCTT TTACATAGCA |
| 251 | ATTTTAGGAT AGACGTAGAG AAGGAACGTT GGTTAAAAAT AAGGGAGTTA |
| 301 | GGAATTGATA TAGAAAGCTG CAAACTCCCC AGTTCTTATG TAAACCAGGT |
| 351 | TTCCTCGTTT ATTTGGTTTG AAAAAGATAA ATCCAAACGG CCACGTATTG |
| 401 | ATGTAGATTA TCATACGCTA CATAGCAAAG ACTGGGTAGT TTTCCCTATC |
| 451 | GTTTTTCAGA AAATTCCAAA GACCTCGCGT TTCAGTTATT GGTTCTCACA |
| 501 | AAAAGAAACA AGGAAGAGGG ATTATGTGAG AAATATGCTG GACCACGTCA |
| 551 | TTGGTTATCT AACGTCAGAA GGTGGGGAGT GGTTGCAGTA TATATCGAAA |
| 601 | ACCTCTTATC AAAGCGCTAC TTCCTTGGAT CCTGAAAGAG TTCTTCAATA |
| 651 | TTGCTTAACT GATAACCAGG AGCTCCAGGG AGAAGTGCAA CGTTTGCTTA |
| 701 | ATGAGGAGAG TGCGACCAAA AGCTCTGGGG ATAAGGAAGT TTTGTTAAGT |
| 751 | CATGTATCTG ACATTATTTG CCAGTGTTGG TGGCCAAAGT TTCTTGAAGT |
| 801 | TATACAATCT CCGGCCTTTA TTGAAGAATT AGTAGAAGAA GTGAGTGGTA |
| 851 | AACTTAATTT AGATTTTTTA TGCCTAGAAA AGGCTAATAC ATTAGATCAG |
| 901 | GAGTTGAGAA ACAGTCTTCT AAGAGCAGTC GTACACCACG GTTCTGAAGG |
| 951 | AGTTGATATT AAGAAAGTTG GTGCCGGCCT CATTATTTAT ACGGAAGCTA |
| 1001 | TTCAATTACA GATTCCCTTC TCAAGGAGTT AA |

The PSORT algorithm predicts inner membrane (0.508).

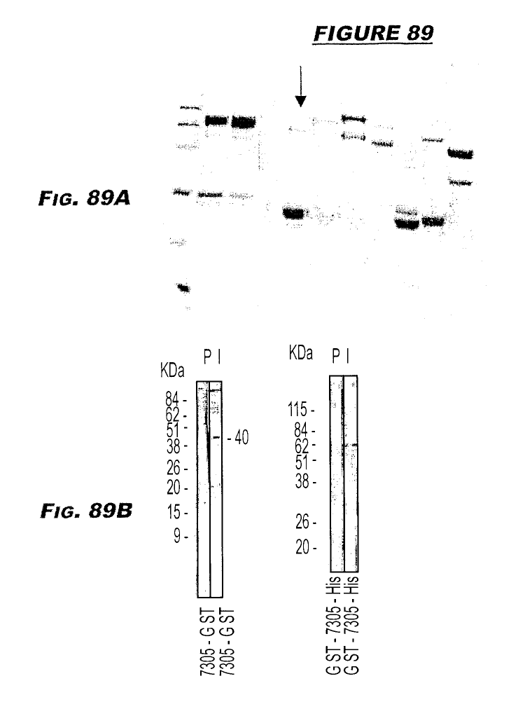

The protein was expressed in E. coli and purified as a GST-fusion product (FIG. 89A) and also as a double GST/his fusion. The recombinant proteins were used to immunize mice, whose sera were used in a Western blot (FIG. 89B) and for FACS analysis.

These experiments show that cp7305 is a surface-exposed and immunoaccessible protein, and that it is a useful immunogen. These properties are not evident from the sequence alone.

### Example 90

The following C. pneumoniae protein (PID 4377347) was expressed <SEQ ID 179; cp7347>:

| | |
|---|---|
| 1 | MKKGKLGAIV FGLLFTSSVA GFSKDLTKDN AYQDLNVIEH LISLKYAPLP |
| 51 | WKELLFGWDL SQQTQQARLQ LVLEEKPTTN YCQKVLSNYV RSLNDYHAGI |
| 101 | TFYRTESAYI PYVLKLSEDG HVFVVDVQTS QGDIYLGDEI LEVDGMGIRE |
| 151 | AIESLRFGRG SATDYSAAVR SLTSRSAAFG DAVPSGIAML KLRRPSGLIR |
| 201 | STPVRWRYTP EHIGDFSLVA PLIPEHKPQL PTQSCVLFRS GVNSQSSSSS |
| 251 | LFSSYMVPYF WEELRVQNKQ RFDSNHHIGS RNGFLPTFGP ILWEQDKGPY |
| 301 | RSYIFKAKDS QGNPHRIGFL RISSYVWTDL EGLEEDHKDS PWELFGEIID |
| 351 | HLEKETDALI IDQTHNPGGS VFYLYSLLSM LTDHPLDTPK HRMIFTQDEV |
| 401 | SSALHWQDLL EDVFTDEQAV AVLGETMEGY CMDMHAVASL QNFSQSVLSS |
| 451 | WVSGDINLSK PMPLLGFAQV RPHPKHQYTK PLFMLIDEDD FSCGDLAPAI |
| 501 | LKDNGRATLI GKPTAGAGGF VFQVTFPNRS GIKGLSLTGS LAVRKDGEFI |
| 551 | ENLGVAPHID LGFTSRDLQT SRFTDYVEAV KTIVLTSLSE NAKKSEEQTS |
| 601 | PQETPEVIRV SYPTTTSAS* |

A predicted signal peptide is highlighted.

The cp7347 nucleotide sequence <SEQ ID 180> is:

| | |
|---|---|
| 1 | ATGAAAAAAG GGAAATTAGG AGCCATAGTT TTTGGCCTTC TATTTACAAG |
| 51 | TAGTGTTGCT GGTTTTTCTA AGGATTTGAC TAAAGACAAC GCTTATCAAG |
| 101 | ATTTAAATGT CATAGAGCAT TTAATATCGT TAAAATATGC TCCTTTACCA |
| 151 | TGGAAGGAAC TATTATTTGG TTGGGATTTA TCTCAGCAAA CACAGCAAGC |
| 201 | TCGCTTGCAA CTGGTCTTAG AAGAAAAACC AACAACCAAC TACTGCCAGA |
| 251 | AGGTACTCTC TAACTACGTG AGATCATTAA ACGATTATCA TGCAGGGATT |
| 301 | ACGTTTTATC GTACTGAAAG TGCGTATATC CCTTACGTAT TGAAGTTAAG |
| 351 | TGAAGATGGT CATGTCTTTG TAGTCGACGT ACAGACTAGC CAAGGGGATA |
| 401 | TTTACTTAGG GGATGAAATC CTTGAAGTAG ATGGAATGGG GATTCGTGAG |
| 451 | GCTATCGAAA GCCTTCGCTT TGGACGAGGG AGTGCCACAG ACTATTCTGC |
| 501 | TGCAGTTCGT TCCTTGACAT CGCGTTCCGC CGCTTTTGGA GATGCGGTTC |
| 551 | CTTCAGGAAT TGCCATGTTG AAACTTCGCC GACCCAGTGG TTTGATCCGT |
| 601 | TCGACACCGG TCCGTTGGCG TTATACTCCA GAGCATATCG GAGATTTTTC |
| 651 | TTTAGTTGCT CCTTTGATTC CTGAACATAA ACCTCAATTA CCTACACAAA |
| 701 | GTTGTGTGCT ATTCCGTTCC GGGGTAAATT CACAGTCTTC TAGTAGCTCT |
| 751 | TTATTCAGTT CCTACATGGT GCCTTATTTC TGGGAAGAAT TGCGGGTTCA |
| 801 | AAATAAGCAG CGTTTTGACA GTAATCACCA TATAGGGAGC CGTAATGGAT |
| 851 | TTTTACCTAC GTTTGGTCCT ATTCTTTGGG AACAAGACAA GGGGCCCTAT |
| 901 | CGTTCCTATA TCTTTAAAGC AAAAGATTCT CAGGGCAATC CCCATCGCAT |
| 951 | AGGATTTTTA AGAATTTCTT CTTATGTTTG GACTGATTTA GAAGGACTTG |
| 1001 | AAGAGGATCA TAAGGATAGT CCTTGGGAGC TCTTTGGAGA GATCATCGAT |
| 1051 | CATTTGGAAA AAGAGACTGA TGCTTTGATT ATTGATCAGA CCCATAATCC |
| 1101 | TGGAGGCAGT GTTTTCTATC TCTATTCGTT ACTATCTATG TTAACAGATC |
| 1151 | ATCCTTTAGA TACTCCTAAA CATAGAATGA TTTTCACTCA GGATGAAGTC |
| 1201 | AGCTCGGCTT TGCACTGGCA AGATCTACTA GAAGATGTCT TCACAGATGA |
| 1251 | GCAGGCAGTT GCCGTGCTAG GGGAAACTAT GGAAGGATAT TGCATGGATA |
| 1301 | TGCATGCTGT AGCCTCTCTT CAAAACTTCT CTCAGAGTGT CCTTTCTTCC |
| 1351 | TGGGTTTCAG GTGATATTAA CCTTTCAAAA CCTATGCCTT TGCTAGGATT |
| 1401 | TGCACAGGTT CGACCTCATC CTAAACATCA ATATACTAAA CCTTTGTTTA |
| 1451 | TGTTGATAGA CGAGGATGAC TTCTCTTGTG GAGATTTAGC GCCTGCAATT |
| 1501 | TTGAAGGATA ATGGCCGCGC TACTCTCATT GGAAAGCCAA CAGCAGGAGC |
| 1551 | TGGAGGTTTT GTATTCCAAG TCACTTTCCC TAACCGTTCT GGAATTAAAG |
| 1601 | GTCTTTCTTT AACAGGATCT TTAGCTGTTA GGAAAGATGG TGAGTTTATT |
| 1651 | GAAAACTTAG GAGTGGCTCC TCATATTGAT TTAGGATTTA CCTCCAGGGA |
| 1701 | TTTGCAAACT TCCAGGTTTA CTGATTACGT TGAGGCAGTG AAAACTATAG |
| 1751 | TTTTAACTTC TTTGTCTGAG AACGCTAAGA AGAGTGAAGA GCAGACTTCT |
| 1801 | CCGCAAGAGA CGCCTGAAGT TATTCGAGTC TCTTATCCCA CAACGACTTC |
| 1851 | TGCTTCGTAA |

The PSORT algorithm predicts periplasmic space (0.2497).

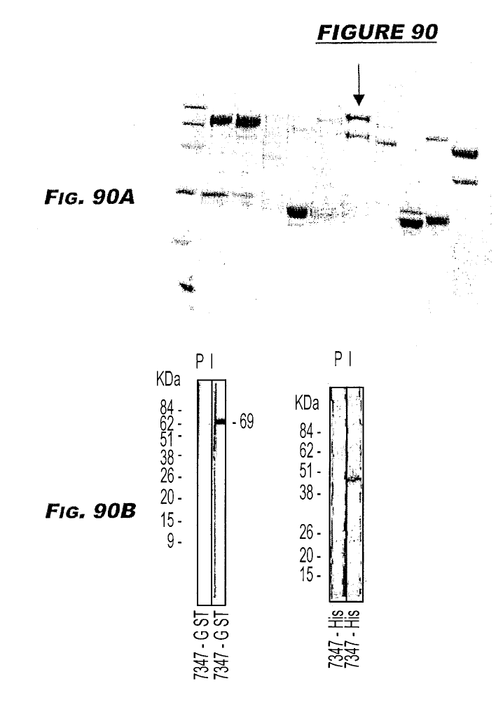

The protein was expressed in E. coli and purified as a GST-fusion product (FIG. 90A) and also in his-tagged form. The recombinant proteins were used to immunize mice, whose sera were used in a Western blot (FIG. 90B) and for FACS analysis.

These experiments show that cp7347 is a surface-exposed and immunoaccessible protein, and that it is a useful immunogen. These properties are not evident from the sequence alone.

### Example 91

The following C. pneumoniae protein (PID 4377353) was expressed <SEQ ID 181; cp7353>:

| | |
|---|---|
| 1 | MNMPVPSAVP SANITLKEDS STVSTASGIL KTATGEVLVS CTALEGSSST |
| 51 | DALISLALGQ IILATQQELL LQSTNVHQLL FLPPEVVELE IQVVDLLVQL |
| 101 | EHAETITSEP QETQTQSRSE QTLPQQSSSK QSALSPRSLK PEISDSKQQQ |
| 151 | ALQTPKDSAV RKHSEAPSPE TQARASLSQA SSSSQRSLPP QESAPERTLL |
| 201 | EQQKASSFSP LSQFSAEKQK EALTTSKSHE LYKERDQDRQ QREQHDRKHD |
| 251 | QEEDAESKKK KKKRGLGVEA VAEEPGENLD IAALIFSDQM RPPAEETSKK |
| 301 | ETTFKKKLPS PMSVFSRFIP SKNPLSVGSS IHGPIQTPKV ENVFLRFMKL |
| 351 | MARILGQAEA EANELYMRVK QRTDDVDTLT VLISKINNEK KDIDWSENEE |
| 401 | MKALLNRAKE IGVTIDKEKY TWTEEEKRLL KENVQMRKEN MEKITQMERT |
| 451 | DMQRHLQEIS QCHQARSNVL KLLKELMDTF IYNLRP* |

The cp7353 nucleotide sequence <SEQ ID 182> is:

| | |
|---|---|
| 1 | ATGAATATGC CTGTTCCTTC TGCAGTTCCC TCTGCAAATA TAACTCTAAA |
| 51 | AGAAGACAGC TCAACAGTTT CCACAGCCTC TGGAATATTA AAGACTGCAA |
| 101 | CAGGTGAAGT CTTAGTCTCT TGTACAGCGC TAGAAGGAAG CTCTTCTACA |
| 151 | GATGCTTTAA TTAGCTTAGC TTTAGGACAA ATCATTCTTG CGACCCAACA |
| 201 | AGAACTGCTC TTACAAAGCA CAAATGTTCA TCAACTCCTC TTCCTCCCTC |
| 251 | CTGAAGTTGT AGAATTAGAA ATCCAAGTTG TTGACTTGCT AGTGCAATTG |
| 301 | GAACATGCAG AGACAATCAC AAGTGAACCA CAAGAAACAC AAACGCAAAG |
| 351 | TAGGAGTGAG CAGACCCTCC CTCAACAAAG CAGCAGTAAA CAATCTGCTC |
| 401 | TCTCCCCACG CTCCTTAAAA CCTGAAATTT CTGATTCTAA ACAACAGCAA |
| 451 | GCTCTTCAAA CACCAAAAGA CTCTGCTGTA AGAAAACACA GCGAAGCACC |
| 501 | GTCACCTGAG ACACAAGCTC GCGCTTCCTT ATCTCAGGCA AGCTCAAGTT |
| 551 | CTCAGAGATC CTTACCTCCG CAAGAAAGTG CGCCAGAAAG AACACTATTA |
| 601 | GAACAACAAA AAGCAAGCTC CTTCTCTCCT CTATCCCAGT TCTCTGCAGA |
| 651 | GAAACAAAAA GAGGCCCTGA CGACCTCAAA ATCTCATGAA CTCTATAAAG |
| 701 | AACGCGATCA AGATCGCCAA CAAAGAGAGC AGCACGACAG AAAGCACGAT |
| 751 | CAGGAAGAAG ACGCTGAATC TAAAAAGAAA AAGAAGAAAC GTGGTCTCGG |
| 801 | TGTAGAGGCA GTCGCTGAGG AACCCGGAGA AAATCTAGAT ATTGCCGCTT |
| 851 | TAATCTTCTC AGATCAAATG CGACCTCCTG CTGAAGAAAC TTCTAAAAAA |
| 901 | GAAACGACAT TCAAAAAGAA GCTACCTTCT CCAATGTCTG TGTTTAGCAG |
| 951 | ATTCATCCCT AGTAAGAATC CGTTATCTGT AGGCTCTTCA ATACACGGGC |
| 1001 | CTATACAAAC TCCAAAAGTA GAAAATGTGT TCTTAAGGTT CATGAAGCTC |
| 1051 | ATGGCAAGAA TCTTAGGCCA AGCCGAAGCC GAAGCTAATG AACTCTACAT |
| 1101 | GCGAGTCAAA CAACGTACCG ATGATGTAGA CACACTCACA GTCCTTATCT |
| 1151 | CTAAGATCAA TAATGAAAAG AAAGACATTG ATTGGAGTGA AAATGAAGAG |
| 1201 | ATGAAAGCTC TTTTAAATCG AGCTAAAGAG ATTGGAGTCA CTATAGACAA |
| 1251 | AGAAAAATAT ACTTGGACAG AAGAGGAAAA AAGACTTCTA AAAGAGAATG |
| 1301 | TCCAAATGCG CAAAGAGAAT ATGGAGAAAA TCACTCAAAT GGAAAGGACG |
| 1351 | GACATGCAAA GGCACCTCCA AGAGATTTCT CAATGTCATC AAGCGCGCTC |
| 1401 | TAATGTATTG AAGTTATTGA AAGAACTTAT GGACACCTTC ATTTACAACC |
| 1451 | TACGCCCCTA A |

The PSORT algorithm predicts cytoplasm (0.1308).

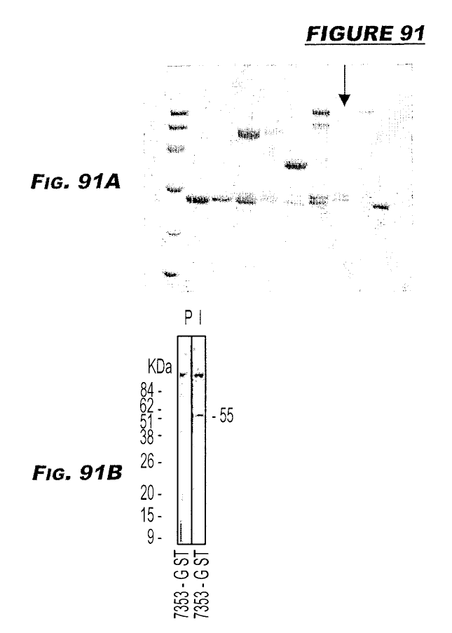

The protein was expressed in E. coli and purified as a GST-fusion product (FIG. 91A). The recombinant protein was used to immunize mice, whose sera were used in a Western blot (FIG. 91B) and for FACS analysis.

These experiments show that cp7353 is a surface-exposed and immunoaccessible protein, and that it is a useful immunogen. These properties are not evident from the sequence alone.

### Example 92

The following C. pneumoniae protein (PID 4377408) was expressed <SEQ ID 183; cp7408>:

| | |
|---|---|
| 1 | MLKIQKKRMC VSVVITVGAI VGFFNSADAA PKKKKIPIQI LYSFTKVSSY |
| 51 | LKNEDASTIF CVDVDRGLLQ HRYLGSPGWQ ETRRRQLFKS LENQSYGNER |
| 101 | LGEETLAIDI FRNKECLESE IPEQMEAILA NSSALVLGIS SFGITGIPAT |
| 151 | LHSLLRQNLS FQKRSIASES FLLKIDSAPS DASVFYKGVL FRGETAIVDA |
| 201 | LSQLFAQLDL SPKKIIFLGE DPEVVQAVGS ACIGWGMNFL GLVYYPAQES |
| 251 | LFSYVHPYST ATELQEAQGL QVISDEVAQL TLNALPKMN* |

The cp7408 nucleotide sequence <SEQ ID 184> is:

| | |
|---|---|
| 1 | ATGTTGAAAA TCCAGAAAAA AAGAATGTGT GTCAGCGTAG TCATCACGGT |
| 51 | AGGCGCCATA GTGGGGTTTT TCAATTCTGC AGACGCAGCA CCAAAGAAAA |
| 101 | AGAAGATCCC TATACAGATT CTCTACTCCT TTACTAAAGT CTCTTCCTAT |
| 151 | TTAAAAAACG AAGACGCAAG TACTATATTT TGCGTCGATG TGGATCGTGG |
| 201 | ACTTCTCCAG CATCGGTATT TAGGTAGTCC AGGATGGCAG GAAACCAGAC |
| 251 | GTCGGCAGTT ATTTAAATCC TTAGAAAATC AATCATACGG CAACGAACGT |
| 301 | TTAGGAGAAG AAACTCTTGC TATTGATATT TTCAGGAACA AAGAGTGCTT |
| 351 | GGAGAGCGAG ATCCCAGAGC AGATGGAAGC TATCCTTGCA AATTCCTCGG |
| 401 | CCTTGGTCTT AGGCATCTCT TCTTTTGGGA TCACAGGAAT TCCTGCGACT |
| 451 | TTGCATAGTT TGCTTCGACA GAATCTATCT TTCCAAAAAC GCTCTATAGC |
| 501 | ATCGGAGAGC TTCCTTTTAA AGATCGATAG TGCCCCCTCA GATGCCTCTG |
| 551 | TTTTTTATAA AGGCGTGCTT TTCCGCGGAG AGACTGCGAT CGTGGATGCG |
| 601 | TTAAGCCAAT TATTTGCCCA GCTCGATCTT TCTCCTAAAA AAATTATCTT |
| 651 | TCTAGGAGAA GACCCTGAGG TCGTTCAAGC TGTTGGGTCT GCTTGTATAG |
| 701 | GTTGGGGCAT GAACTTTTTA GGCCTGGTAT ACTATCCTGC TCAAGAAAGC |
| 751 | CTTTTTTCTT ATGTTCATCC TTACTCTACA GCAACGGAGC TCCAAGAAGC |
| 801 | ACAGGGTTTA CAAGTAATTT CAGATGAAGT CGCACAGCTT ACTTTAAACG |
| 851 | CTCTTCCGAA AATGAATTAA |

The PSORT algorithm predicts inner membrane (0.123).

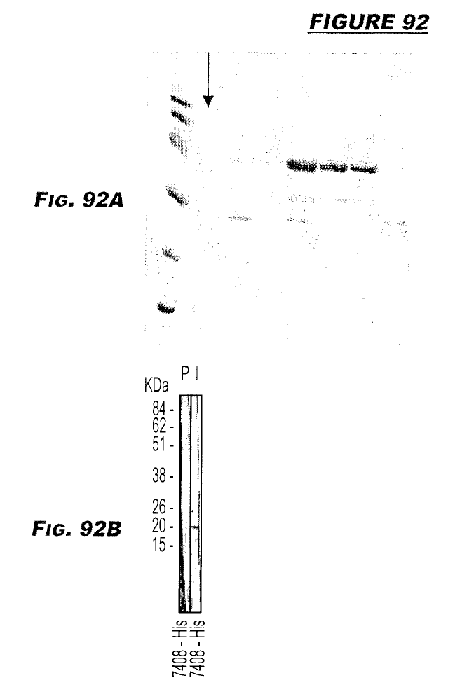

The protein was expressed in E. coli and purified as a his-tag product (FIG. 92A). The recombinant protein was used to immunize mice, whose sera were used in a Western blot (FIG. 92B) and for FACS analysis.

These experiments show that cp7408 is a surface-exposed and immunoaccessible protein, and that it is a useful immunogen. These properties are not evident from the sequence alone.

### Example 93

The following C. pneumoniae protein (PID 4376424) was expressed <SEQ ID 185; cp6424>:

| | |
|---|---|
| 1 | MMHNIVVLSE EPGRSAFLGR TAFFPNKYPI AQGGVGIPST IGNLFTIWYC |
| 51 | FYFYRAATPQ SDHPDGCGFI LLERLKELGA GFFYCDLRES NTTGFTLFFE |
| 101 | GSNKGVLKNH LFIRDE* |

The cp6424 nucleotide sequence <SEQ ID 186> is:

| | |
|---|---|
| 1 | ATGATGCACA ATATTGTTGT TCTTAGTGAG GAACCTGGAC GAAGCGCTTT |
| 51 | TCTTGGTAGG ACGGCATTTT TCCCTAATAA GTATCCAATA GCTCAGGGTG |
| 101 | GTGTTGGAAT ACCATCTACA ATAGGCAATC TCTTTACTAT ATGGTACTGT |
| 151 | TTCTATTTTT ATAGAGCTGC AACTCCACAA TCTGATCATC CTGACGGATG |
| 201 | TGGCTTTATT CTACTAGAAA GGCTTAAGGA GCTCGGTGCA GGGTTCTTTT |
| 251 | ATTGTGATCT TCGTGAGTCC AATACCACTG GCTTTACTCT TTTTTTTGAA |
| 301 | GGCTCCAATA AAGGTGTGTT AAAGAATCAC TTGTTTATTA GAGATGAGTA |
| 351 | A |

The PSORT algorithm predicts cytoplasm (0.2502).

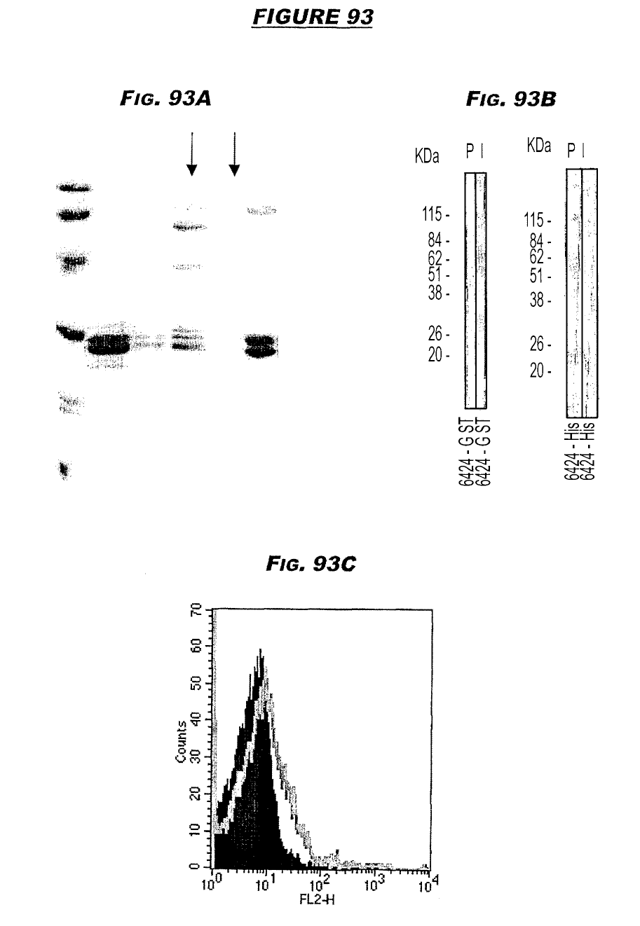

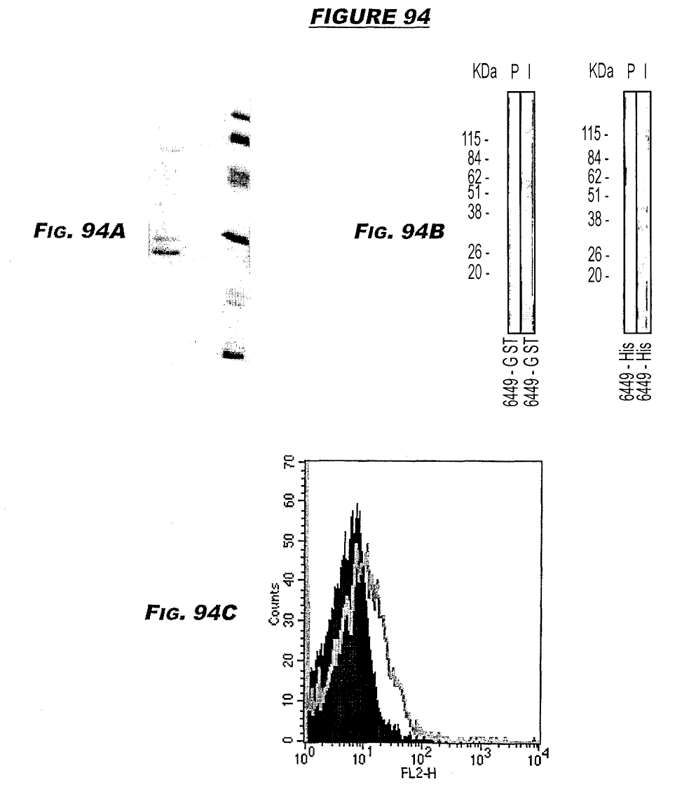

The protein was expressed in E. coli and purified as a GST-fusion product (FIG. 93A) and also in his-tagged form. The recombinant proteins were used to immunize mice, whose sera were used in Western blots (FIG. 93B) and for FACS analyses (FIG. 93C; GST-fusion).

These experiments show that cp6424 is a surface-exposed and immunoaccessible protein, and that it is a useful immunogen. These properties are not evident from the sequence alone.

### Example 94

The following C. pneumoniae protein (PID 4376449) was expressed <SEQ ID 187; cp6449>:

```
    1 VASETYPSQI LHAQREVRDA YFNQADCHPA RANQILEAKK ICLLDVYHTN
  51 HYSVFTFCVD NYPNLRFTFV SSKNNEMNGL SNPLDNVLVE AMVRRTHARN
101 LLAACKIRNI EVPRVVGLDL RSGILISKLE LKQPQFQSLT EDFVNHSTNQ
151 EEARVHQKHV LLISLILLCK QAVLESFQEK KRSS*
```

The cp6449 nucleotide sequence <SEQ ID 188> is:

```
    1 GTGGCGTCTG AAACGTATCC TTCTCAGATA TTGCACGCTC AGAGGGAAGT
  51 ACGTGATGCC TATTTTAATC AAGCGGATTG CCATCCTGCT CGGGCTAATC
101 AGATTCTCGA GGCTAAGAAA ATCTGTTTAT TAGATGTTTA TCATACTAAT
151 CATTATTCCG TATTTACTTT TTGTGTAGAT AATTATCCGA ATCTCCGCTT
201 TACATTTGTA TCTTCAAAAA ACAATGAGAT GAATGGCTTA TCTAATCCTC
251 TAGATAATGT TCTTGTAGAG GCTATGGTAC GTAGAACACA TGCAAGAAAC
301 CTACTTGCAG CGTGTAAAAT TCGAAATATT GAGGTTCCAA GGGTTGTTGG
351 GCTTGACCTA AGATCTGGGA TACTCATTTC GAAACTAGAA TTGAAGCAAC
401 CTCAGTTCCA AAGTTTAACA GAAGACTTCG TAAATCATTC CACAAATCAG
451 GAAGAAGCTC GCGTCCATCA AAAGCATGTG TTGCTAATTT CTTTAATTTT
501 ACTTTGCAAG CAGGCCGTTC TGGAATCATT CCAGGAAAAA AAGCGATCCT
551 CTTAA
```

The PSORT algorithm predicts inner membrane (0.2084).

The protein was expressed in E. coli and purified as a GST-fusion product (FIG. 94A) and also in his-tagged form. The recombinant proteins were used to immunize mice, whose sera were used in Western blots (FIG. 94B) and for FACS analyses (FIG. 94C; GST-fusion).

These experiments show that cp6449 is a surface-exposed and immunoaccessible protein, and that it is a useful immunogen. These properties are not evident from the sequence alone.

### Example 95

The following C. pneumoniae protein (PID 4376495) was expressed <SEQ ID 189; cp6495>:

MRELNAFELTQPEEYRNRWVLMPCLKCRFCRTQHAKVWSYRCVHEASLYE
KNCFLTLTYDDKHLPQYGSLVKLHLQLFLKRLRKMISPHKIRYFECGAYG
TKLQRPHYHLLLS

The cp6495 nucleotide sequence <SEQ ID 190> is:

TTGCGAGAATTAATGCTTTTGAATTAACTCAACCTGAAGAGTATCGAAAC
CGTTGGGTTTTGATGCCTTGTCTTAAGTGTCGTTTTTGTAGAACGCAACA
TGCAAAAGTCTGGTCTTATCGTTGTGTCCATGAAGCTTCTTTGTATGAGA
AAAATTGTTTTCTTACTTTGACTTATGATGATAAGCATTTACCTCAGTAT
GGTTCGTTGGTAAAGCTGCATTTACAGCTGTTTCTTAAGAGATTAAGAAA
GATGATTTCTCCTCATAAAATTCGTTATTTTGAATGTGGTGCGTATGGAA
CCAAATTACAAAGACCTCATTATCATCTACTTTTATCATGA

The PSORT algorithm predicts cytoplasmic (0.280).

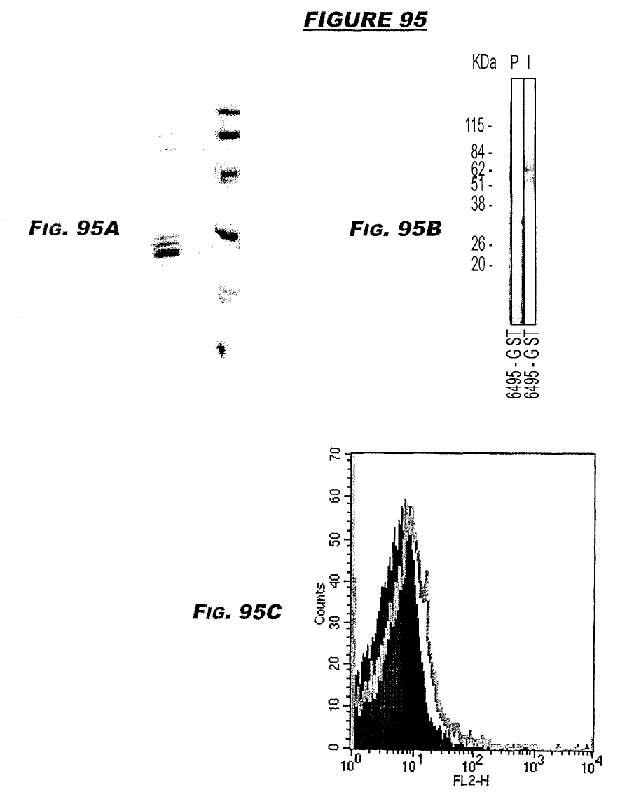

The protein was expressed in E. coli and purified as a GST-fusion product (FIG. 95A). The recombinant protein was used to immunize mice, whose sera were used in a Western blot (FIG. 95B) and for FACS analysis (FIG. 95C).

These experiments show that cp6495 is a surface-exposed and immunoaccessible protein, and that it is a useful immunogen. These properties are not evident from the sequence alone.

### Example 96

The following C. pneumoniae protein (PID 4376506) was expressed <SEQ ID 191; cp6506>:

```
    1 MRRFLFLILS SLPLVAFSAD NFTILEEKQS PLSRVSIIFA LPGVTPVSFD
  51 GNCPIPWFSH SKKTLEGQRI YYSGDSFGKY FVVSALWPNK VSSAVVACNM
101 ILKHRVDLIL IIGSCYSRSQ DSRFGSVLVS KGYINYDADV RPFFERFEIP
151 DIKKSVFATS EVHREAILRG GEEFISTHKQ EIEELLKTHG YLKSTTKTEH
201 TLMEGLVATG ESFAMSRNYF LSLQKLYPEI HGFDSVSGAV SQVCYEYSIP
251 CLGVNILLPH PLESRSNEDW KHLQSEASKI YMDTLLKSVL KELCSSH*
```

The cp6506 nucleotide sequence <SEQ ID 192> is:

```
    1 ATGCGTCGTT TTCTGTTTCT TATTCTTAGC TCTCTTCCTT TGGTCGCATT
  51 CTCTGCTGAT AATTTCACTA TTCTAGAAGA AAAACAGAGT CCTTTAAGTC
101 GTGTAAGTAT TATTTTTGCT TTACCTGGGG TTACTCCCGT TTCTTTTGAT
151 GGTAATTGTC CTATTCCTTG GTTTTCTCAT AGTAAAAAGA CTCTAGAGGG
201 ACAGAGAATT TATTACTCTG GCGACTCCTT TGGGAAATAC TTTGTAGTTT
251 CTGCTCTTTG GCCTAATAAA GTTTCTTCAG CTGTTGTGGC TTGTAATATG
301 ATTCTTAAAC ATCGAGTGGA TCTTATTCTA ATTATAGGCT CGTGTTACTC
351 TAGGTCTCAA GATAGCCGTT TTGGCAGCGT CTTAGTTTCT AAAGGCTACA
401 TTAATTATGA TGCAGATGTG AGGCCTTTCT TTGAAAGATT TGAGATTCCA
451 GACATTAAAA AGAGTGTTTT TGCAACCAGT GAGGTTCATC GGGAGGCAAT
501 TCTTCGTGGA GGCGAAGAGT TTATTTCTAC CCATAAACAA GAAATCGAAG
551 AGCTTTTGAA GACTCATGGG TATTTGAAAT CAACAACCAA AACGGAGCAC
501 ACCTTAATGG AAGGTTTGGT TGCTACAGGC GAGTCTTTCG CGATGTCGCG
651 AAACTATTTT CTTTCCTTAC AAAAATTGTA TCCAGAGATT CATGGTTTTG
701 ATAGTGTCAG CGGCGCTGTT TCTCAGGTAT GCTATGAATA TAGCATTCCT
751 TGTTTAGGTG TGAATATCCT TCTCCCTCAT CCTTTAGAAT CACGGAGTAA
801 CGAGGATTGG AAGCATCTTC AAAGTGAGGC AAGTAAAATT TATATGGATA
851 CCTTGCTCAA GAGTGTATTA AAAGAACTCT GTTCTTCTCA TTAA
```

The PSORT algorithm predicts periplasmic space (0.571).

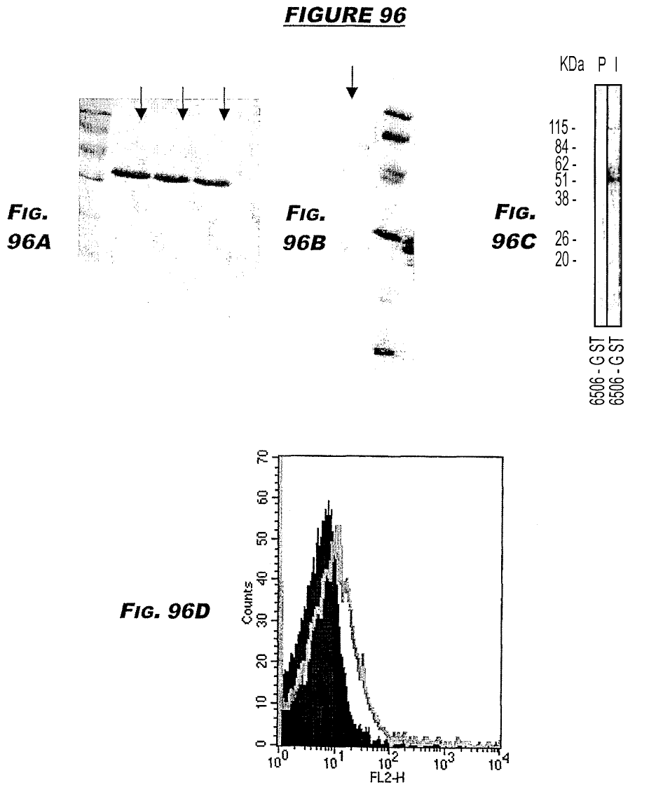

The protein was expressed in E. coli and purified as his-tag (FIG. 96A) and GST-fusion (FIG. 96B) products. The GST-fusion protein was used to immunize mice, whose sera were used in a Western blot (FIG. 96C) and for FACS analysis (FIG. 96D).

These experiments show that cp6506 is a surface-exposed and immunoaccessible protein, and that it is a useful immunogen. These properties are not evident from the sequence alone.

### Example 97

The following C. pneumoniae protein (PID 4376882) was expressed <SEQ ID 193; cp6882>:

```
    1 MSLLNLPSSQ DSASEDSTSQ SQIFDPIRNR ELVSTPEEKV RQRLLSFLMH
  51 KLNYPKKLII IEKELKTLFP LLMRKGTLIP KRRPDILIIT PPTYTDAQGN
101 THNLGDPKPL LLIECKALAV NQNALKQLLS YNYSIGATCI AMAGKHSQVS
151 ALFNPKTQTL DFYPGLPEYS QLLNYFISLN L*
```

The cp6882 nucleotide sequence <SEQ ID 194> is:

```
    1 ATGTCCTTAT TGAACCTTCC CTCAAGCCAG GATTCTGCAT CTGAGGACTC
  51 CACATCGCAA TCTCAAATCT TCGATCCCAT TAGAAATCGG GAGTTAGTTT
101 CTACTCCCGA AGAAAAAGTC CGCCAAAGGT TGCTCTCCTT CCTAATGCAT
151 AAGCTGAACT ACCCTAAGAA ACTCATCATC ATAGAAAAAG AACTCAAAAC
201 TCTTTTTCCT CTGCTTATGC GTAAAGGAAC CCTAATCCCA AAACGCCGCC
251 CAGATATTCT CATCATCACT CCCCCCACAT ACACAGACGC ACAGGGAAAC
301 ACTCACAACC TAGGCGACCC AAAACCCCTG CTACTTATCG AATGTAAGGC
351 CTTAGCCGTA AACCAAAATG CACTCAAACA ACTCCTTAGC TATAACTACT
401 CTATCGGAGC CACCTGCATT GCTATGGCAG GGAAACACTC TCAAGTGTCA
451 GCTCTCTTCA ATCCAPAAAC ACAAACTCTT GATTTTTATC CTGGCCTCCC
501 AGAGTATTCC CAACTCCTAA ACTACTTTAT TTCTTTAAAC TTATAG
```

The PSORT algorithm predicts cytoplasm (0.362).

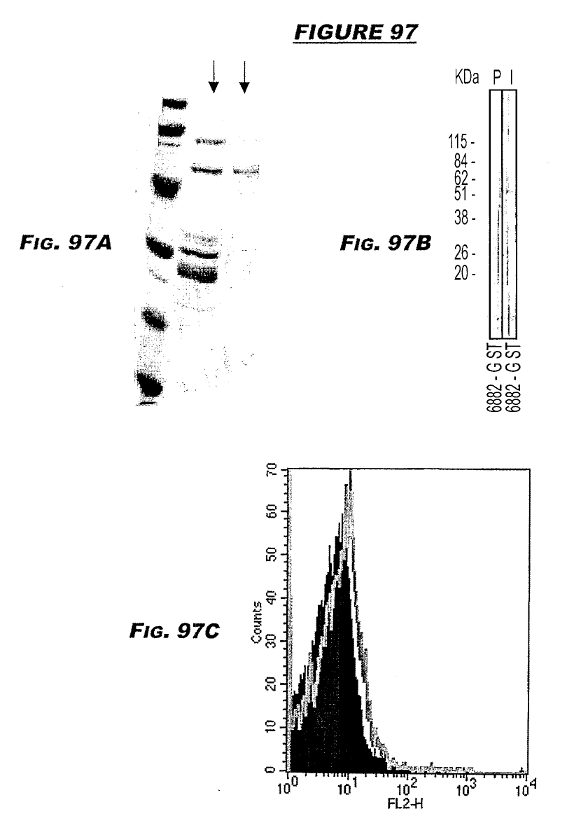

The protein was expressed in E. coli and purified as a GST-fusion product (FIG. 97A). The protein was used to immunize mice, whose sera were used in a Western blot (FIG. 97B) and for FACS analysis (FIG. 97C).

These experiments show that cp6882 is a surface-exposed and immunoaccessible protein, and that it is a useful immunogen. These properties are not evident from the sequence alone.

### Example 98

The following C. pneumoniae protein (PID 4376979) was expressed <SEQ ID 195; cp6979>:

```
    1 MSVNPSGNSK NDLWITGAHD QHPDVKESGV TSANLGSHRV TASGGRQGLL
  51 ARIKEAVTGF FSRMSFFRSG APRGSQQPSA PSADTVRSPL PGGDARATEG
101 AGRNLIKKGY QPGMKVTIPQ VPGGGAQRSS GSTTLKPTRP APPPPKTGGT
151 NAKRPATHGK GPAPQPPKTG GTNAKPAATH GKGPAPQPPK GILKQPGQSG
201 TSGKKRVSWS DED*
```

The cp6979 nucleotide sequence <SEQ ID 196> is:

```
    1 ATGTCTGTTA ATCCATCAGG AAATTCCAAG AACGATCTCT GGATTACGGG
  51 AGCTCATGAT CAGCATCCCG ATGTTAAAGA ATCCGGGGTT ACAAGTGCTA
101 ACCTAGGAAG TCATAGAGTG ACTGCCTCAG GAGGACGCCA AGGGTTATTA
151 GCACGAATCA AAGAAGCAGT AACCGGGTTT TTTAGTCGGA TGAGCTTCTT
201 CAGATCGGGA GCTCCAAGAG GTAGCCAACA ACCCTCTGCT CCATCTGCAG
251 ATACTGTACG TAGCCCGTTG CCGGGAGGGG ATGCTCGCGC TACCGAGGGA
301 GCTGGTAGGA ACTTAATTAA AAAAGGGTAC CAACCAGGGA TGAAAGTCAC
351 TATCCCACAG GTTCCTGGAG GAGGGGCCCA ACGTTCATCA GGTAGCACGA
401 CACTAAAGCC TACGCGTCCG GCACCCCCAC CTCCTAAAAC GGGTGGAACT
451 AATGCAAAAC GTCCGGCAAC GCACGGGAAG GGTCCAGCAC CCCAGCCTCC
501 TAAAACAGGT GGGACCAATG CTAAGCGCGC AGCAACGCAT GGGAAAGGTC
551 CAGCACCTCA ACCTCCTAAG GGCATTTTGA AACAGCCTGG GCAGTCTGGG
601 ACTTCAGGAA AGAAGCGTGT CAGCTGGTCT GACGAAGATT AA
```

The PSORT algorithm predicts cytoplasm (0.360).

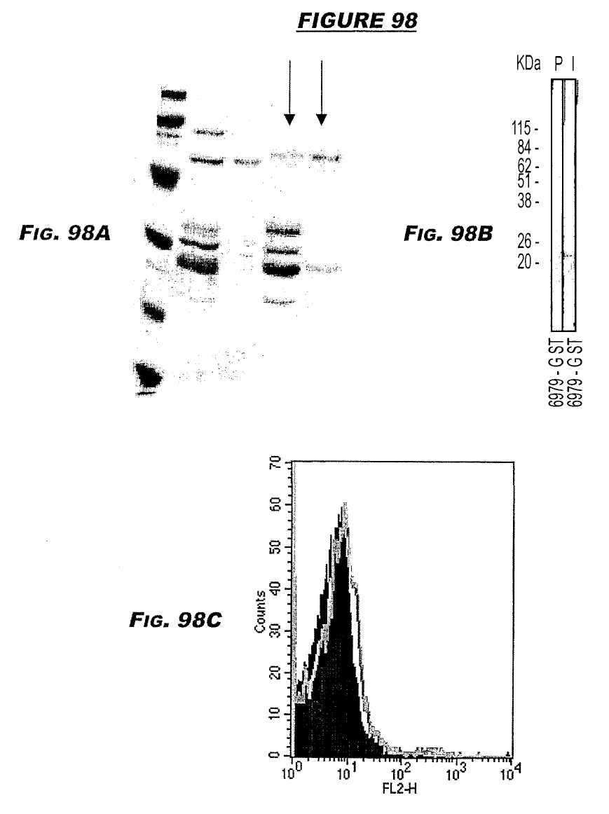

The protein was expressed in E. coli and purified as a GST-fusion product (FIG. 98A). The GST-fusion protein was used to immunize mice, whose sera were used in a Western blot (FIG. 98B) and for FACS analysis (FIG. 98C).

These experiments show that cp6979 is a surface-exposed and immunoaccessible protein, and that it is a useful immunogen. These properties are not evident from the sequence alone.

### Example 99

The following C. pneumoniae protein (PID 4377028) was expressed <SEQ ID 197; cp7028>:

```
    1 MLLGFLCDCP CASWQCAAVA NCYDSVFMSR PEHKPNIPYI TKATRRGLRM
  51 KTLAYLASLK DARQLAYDFL KDPGSLARLA KALIAPKEAL QEGNLFFYGC
101 SNIEDILEEM RRPHRILLLG FSYCQKPKAC PEGRFNDACR YDPSHPTCAS
151 CSIGTMMRLN ARRYTTVIIP TFIDIAKHLH TLKKRYPGYQ ILFAVTACEL
201 SLKMFGDYAS VMNLKGVGIR LTGRICNTFK AFKLAERGVK PGVITLEEDG
251 FEVLARILTE YSSAPFPRDF CEIH*
```

The cp7028 nucleotide sequence <SEQ ID 198> is:

```
    1 ATGCTTCTAG GGTTTTTGTG TGACTGCCCC TGTGCTTCGT GGCAGTGTGC
  51 GGCCGTTGCT AATTGTTATG ATTCCGTATT TATGTCTAGA CCAGAGCACA
101 AACCTAATAT TCCTTATATT ACTAAAGCTA CAAGACGGGG TCTGCGTATG
151 AAGACGCTTG CTTATCTGGC CTCTTTAAAA GATGCTAGAC AGCTTGCCTA
201 TGATTTTCTG AAAGATCCTG GTTCTTTAGC TCGGTTAGCT AAGGCTTTGA
251 TAGCTCCTAA GGAGGCCTTA CAGGAGGGCA ACCTATTTTT TTATGGCTGT
301 AGTAATATTG AGGATATTTT AGAGGAGATG CGTCGTCCTC ATAGAATCCT
351 TTTGTTAGGA TTTTCTTATT GTCAAAAGCC TAAGGCATGT CCTGAAGGGC
401 GTTTCAATGA TGCTTGTCGG TATGATCCTT CACATCCTAC ATGTGCCTCA
451 TGTTCTATAG GGACCATGAT GCGGCTGAAT GCTCGTAGAT ACACTACTGT
501 GATCATCCCT ACATTTATAG ATATCGCAAA ACATTTACAC ACTTTAAAAA
551 AGCGCTACCC TGGATATCAA ATTCTCTTTG CAGTTACTGC TTGTGAACTT
601 TCCTTAAAAA TGTTTGGAGA TTATGCCTCC GTAATGAACT TAAAGGGTGT
651 GGGCATCAGA CTCACAGGAC GTATTTGCAA TACATTTAAG GCATTTAAAT
701 TAGCTGAGCG AGGAGTCAAA CCAGGAGTCA CTATCCTAGA AGAAGATGGC
751 TTTGAGGTAT TAGCAAGGAT TCTTACAGAA TACAGTAGCG CTCCTTTCCC
801 TAGAGACTTT TGTGAGATCC ATTAG
```

The PSORT algorithm predicts cytoplasm (0.1453).

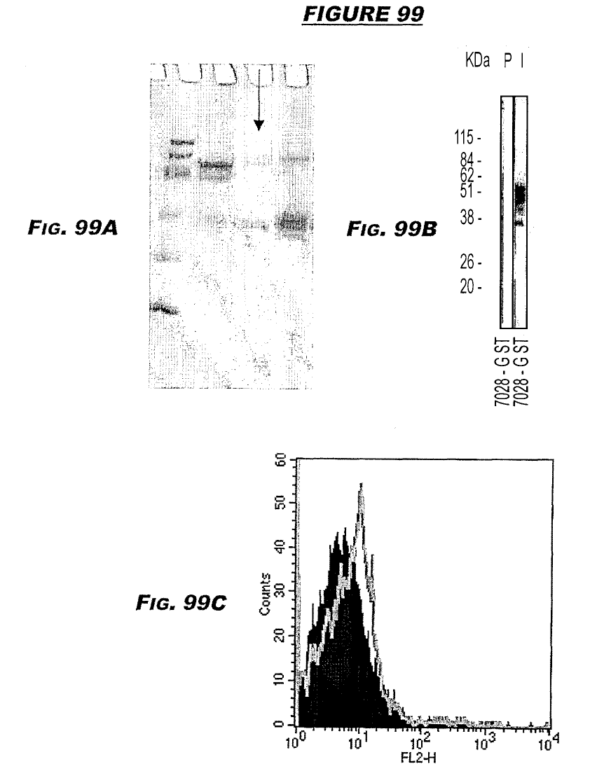

The protein was expressed in E. coli and purified as a GST-fusion product (FIG. 99A). The recombinant protein was used to immunize mice, whose sera were used in a Western blot (FIG. 99B) and for FACS analysis (FIG. 99C).

These experiments show that cp7028 is a surface-exposed and immunoaccessible protein, and that it is a useful immunogen. These properties are not evident from the sequence alone.

### Example 100

The following C. pneumoniae protein (PID 4377355) was expressed <SEQ ID 199; cp7355>:

1 MKKVVTLSII FEATYCASEL SAYTYVAVEL SEAPGKIQVR PVVGLQFQEE
51 QGSVPYSFYY PYDYGYYYPE TYGYTKNTGQ ESRECYTREE DGTIFYECD*

The cp7355 nucleotide sequence <SEQ ID 200> is:

```
    1 ATGAAGAAAG TCGTAACACT ATCCATTATA TTTTTCGCAA CGTATTGTGC
  51 ATCAGAGCTT AGTGCTGTAA CTGTAGTGGC TGTGCCTTTA TCAGAGGCTC
101 CAGGGAAGAT TCAAGTTCGT CCCGTCGTTG GTCTGCAATT TCAAGAAGAA
151 CAGGGTTCTG TGCCCTATAG TTTTTATTAT CCTTATGACT ATGGGTATTA
201 CTATCCAGAG ACTTATGGCT ATACTAAAAA TACAGGTCAA GAAAGTCGCG
251 AATGTTATAC CCGATTTGAA GATGGCACAA TTTTTTATGA ATGCGATTAG
```

The PSORT algorithm predicts inner membrane (0.143).

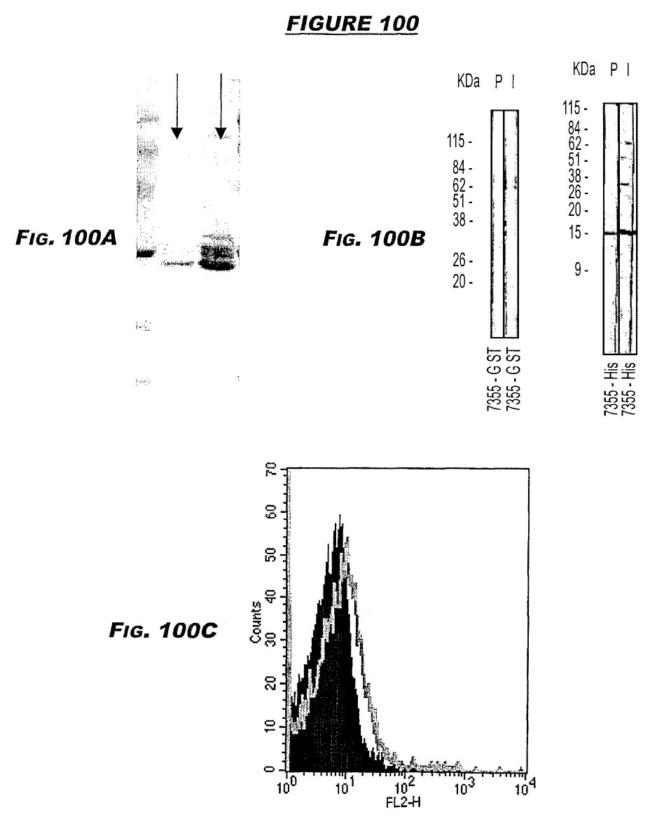

The protein was expressed in E. coli and purified as a GST-fusion (FIG. 100A) and a his-tag product. The proteins were used to immunize mice, whose sera were used in a Western blot (FIG. 100B) and for FACS analysis (FIG. 100C).

These experiments show that cp7355 is a surface-exposed and immunoaccessible protein, and that it is a useful immunogen. These properties are not evident from the sequence alone.

### Example 101

The following C. pneumoniae protein (PID 4377380) was expressed <SEQ ID 201; cp7380>:

```
    1 VHYCERTLDP KYILKIALKL RQSLSLFFQN SQSLQRAYST PYSYYRIILQ
  51 KENKEKQALA RHKCISILEF FKNLLFVHLL SLSKNQREGC STDMAVVSTP
101 FFNRNLWYRL LSSRFSLWKS YCPRFFLDYL EAFGLLSDFL DHQAVIKFFE
151 LETHFSYYPV SGFVAPHQYL SLLQDRYFPI ASVMRTLDKD NFSLTPDLIH
201 DLLGHVPWLL HESESEFFIN MGRLFTKVIE KVQALPSKKQ RIQTLQSNLI
251 AIVRCFWFTV ESGLIENHEG RKAYGAVLIS SPQELGHAFI DNVRVLPLEL
301 DQIIRLPFNT STEQETLESI RHEDELVELT SKLEWMLDQG LLESIPLYNQ
351 EKYLSGFEVL CQ*
```

The cp7380 nucleotide sequence <SEQ ID 202> is:

```
      1 GTGCACTACT GCGAGAGAAC CCTGGACCCA AAGTATATTC TGAAGATTGC
    51 TCTAAAGCTG AGACAATCAC TTTCCCTGTT CTTCCAGAAC AGCCAATCAC
  101 TCCAACGTGC ATACTCGACC CCATATTCCT ACTACCGAAT CATTCTACAA
  151 AAGGAAAATA AAGAGAAGCA AGCTTTAGCT CGACACAAAT GCATTTCTAT
  201 TTTAGAATTT TTCAAAAACT TACTCTTTGT TCATCTTCTG TCATTATCAA
  251 AGAATCAAAG GGAAGGTTGC TCCACTGATA TGGCTGTTGT AAGCACTCCC
  301 TTTTTTAATC GGAATTTATG GTATCGACTC CTTTCCTCAC GGTTTTCTCT
  351 ATGGAAAAGC TATTGTCCAA GATTTTTTCT TGATTACTTA GAAGCTTTCG
  401 GTCTCCTTTC TGATTTCTTA GACCATCAAG CAGTCATTAA ATTCTTCGAA
  451 TTAGAAACAC ATTTTTCCTA TTATCCCGTT TCAGGATTTG TAGCTCCCCA
  501 TCAATACTTG TCTCTGTTGC AGGACCGTTA CTTTCCCATT GCCTCTGTAA
  551 TGCGAACTCT CGATAAAGAT AATTTCTCCT TAACTCCTGA TCTCATCCAT
  601 GACCTTTTAG GGCACGTGCC TTGGCTTCTA CATCCCTCAT TTTCTGAATT
  651 TTTCATAAAC ATGGGAAGAC TCTTCACTAA AGTCATAGAA AAAGTACAAG
  701 CTCTTCCTAG TAAAAAACAA CGCATACAAA CCCTACAAAG CAATCTGATC
  751 GCTATTGTAC GCTGCTTTTG GTTTACTGTT GAAAGCGGAC TTATTGAAAA
  801 CCATGAAGGA AGAAAAGCAT ATGGAGCCGT TCTTATCAGT TCTCCTCAGG
  851 AACTTGGACA CGCTTTCATT GATAACGTAC GTGTTCTCCC TTTAGAATTG
  901 GATCAGATTA TTCGTCTTCC CTTCAATACA TCAACTCCAC AAGAGACTTT
  951 ATTTTCAATA AGACATTTTG ATGAACTGGT AGAACTCACT TCAAAATTAG
1001 AATGGATGCT CGACCAAGGT CTGTTAGAAT CAATTCCCCT TTACAATCAA
1051 GAGAAATATC TTTCTGGTTT TGAGGTACTT TGCCAATGA
```

The PSORT algorithm predicts inner membrane (0.1362).

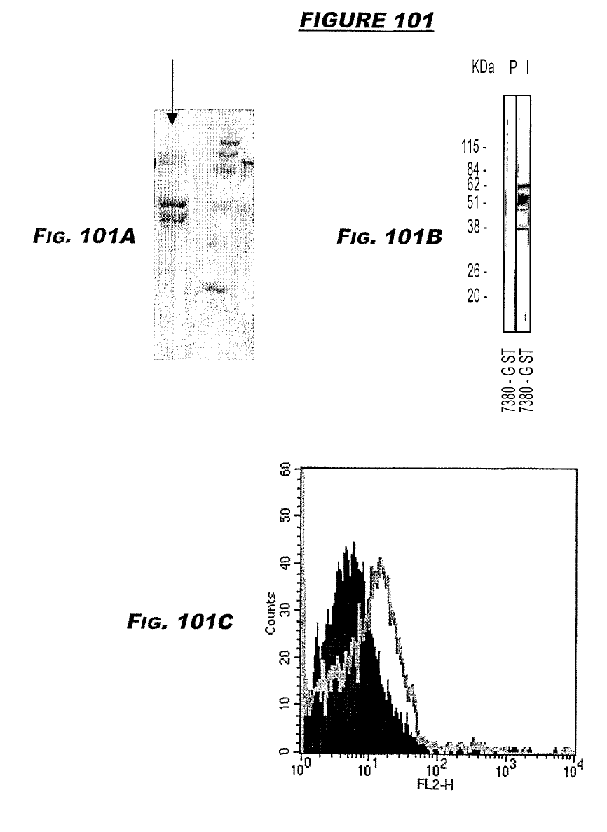

The protein was expressed in E. coli and purified as a GST-fusion product (FIG. 101A). The recombinant protein was used to immunize mice, whose sera were used in a Western blot (FIG. 101B) and for FACS analysis (FIG. 101C).

These experiments show that cp7380 is a surface-exposed and immunoaccessible protein, and that it is a useful immunogen. These properties are not evident from the sequence alone.

### Example 102

The following C. pneumoniae protein (PID 4376904) was expressed <SEQ ID 203; cp6904>:

```
    1 MMNYEDAKLR GQAVAILYQI GAIKFGKHIL ASGEETPLYV DMRLVISSPE
  51 VLQTVATLIW RLRPSFNSSL LCGVPYTALT LATSISLKYN IPMVLRRKEL
101 QNVDPSDAIK VEGLFTPGQT CLVINDMVSS GKSIIETAVA LEENGLVVRE
151 ALVFLDRRKE ACQPLGPQGI KVSSVFTVPT LIKALIAYGK LSSGDLTLAN
201 KISEILEIES *
```

The cp6904 nucleotide sequence <SEQ ID 204> is:

```
    1 ATGATGAACT ACGAAGATGC AAAATTACGC GGTCAAGCTG TAGCAATTCT
  51 ATACCAAATC GGAGCTATAA AGTTCGGAAA ACATATTCTC GCTAGCGGAG
101 AAGAAACTCC TCTGTATGTA GATATGCGTC TTGTGATCTC CTCTCCAGAA
151 GTTCTCCAGA CAGTGGCAAC TCTTATTTGG CGCCTCCGCC CCTCATTCAA
201 TAGTAGCTTA CTCTGCGGAG TCCCTTATAC TGCTCTAACC CTAGCAACCT
251 CGATCTCTTT AAAATATAAC ATCCCTATGG TATTGCGAAG GAAGGAATTA
301 CAGAATGTAG ACCCCTCGGA CGCTATTAAA GTAGAAGGGT TATTTACTCC
351 AGGACAAACT TGTTTAGTCA TCAATGATAT GGTTTCCTCA GGAAAATCTA
401 TAATAGAGAC AGCAGTCGCA CTGGAAGAAA ATGGTCTGGT AGTTCGTGAA
451 GCATTGGTAT TCTTAGATCG TAGAAAAGAA GCGTGTCAAC CACTTGGTCC
501 ACAGGGAATA AAAGTCAGTT CGGTATTTAC TGTACCCACT CTGATAAAAG
551 CTTTGATCGC TTATGGGAAG CTAAGCAGTG GTGATCTAAC CCTGGCAAAC
601 AAAATTTCCG AAATTCTAGA AATTGAATCT TAA
```

The PSORT algorithm predicts cytoplasm (0.0358).

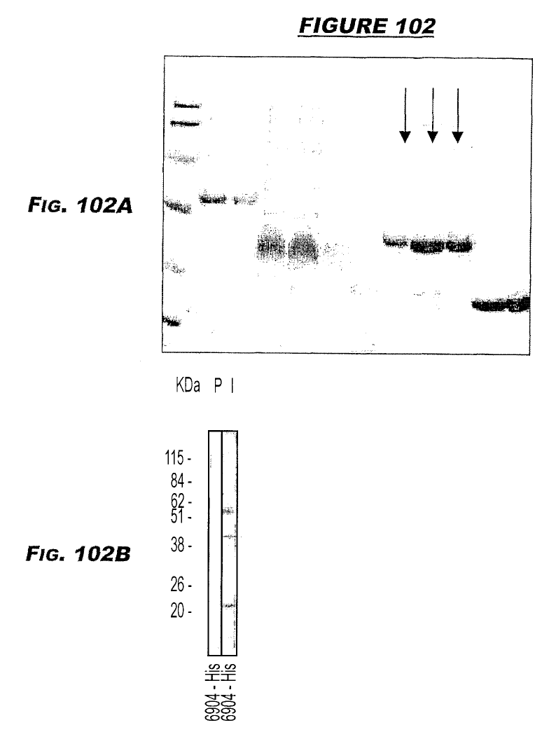

The protein was expressed in E. coli and purified as a his-tag product (FIG. 102A). The recombinant protein was used to immunize mice, whose sera were used in a Western blot (FIG. 102B) and for FACS analysis.

The cp6904 protein was also identified in the 2D-PAGE experiment.

These experiments show that cp6904 is a surface-exposed and immunoaccessible protein, and that it is a useful immunogen. These properties are not evident from the sequence alone.

### Example 103

The following C. pneumoniae protein (PID 4376964) was expressed <SEQ ID 205; cp6964>:

1 MKKLIALIGI FLVPIKGNTN KEHDAHATVL KAARAKYNLF FVQDVFPVHE
51 VIEPISPDCL VHYEGWV*

The cp6964 nucleotide sequence <SEQ ID 206> is:

```
    1 ATGAAAAAAT TGATTGCTTT GATAGGGATA TTTCTTGTTC CAATAAAAGG
  51 AAATACCAAT AAGGAACACG ACGCTCACGC GACTGTTTTA AAAGCGGCCA
101 GAGCAAAGTA TAATTTGTTC TTTGTTCAGG ATGTTTTCCC TGTACACGAA
151 GTTATCGAGC CTATTTCTCC CGATTGCCTG GTACATTATG AAGGGTGGGT
201 TTGA
```

The PSORT algorithm predicts inner membrane (0.091).

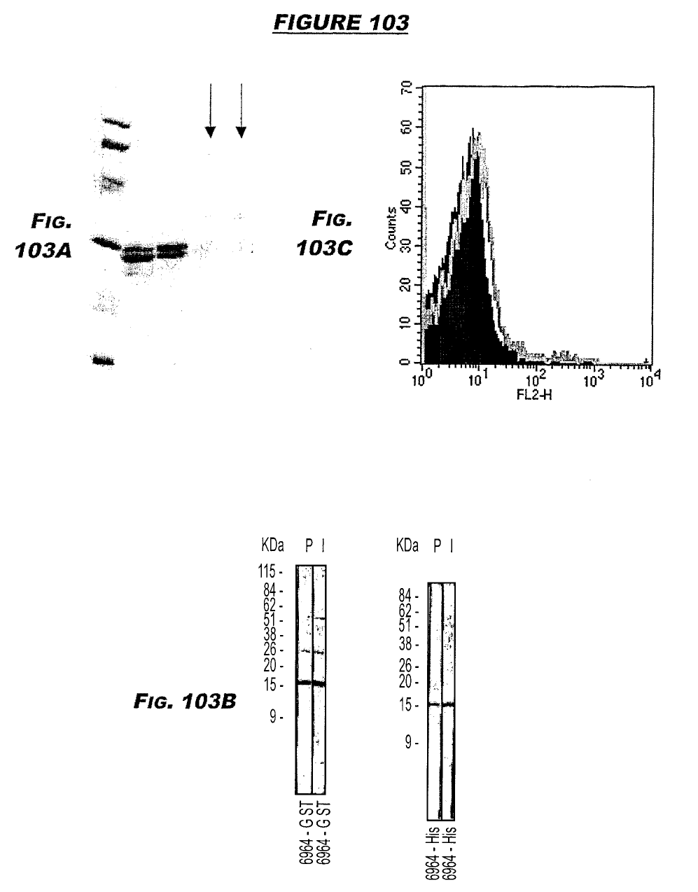

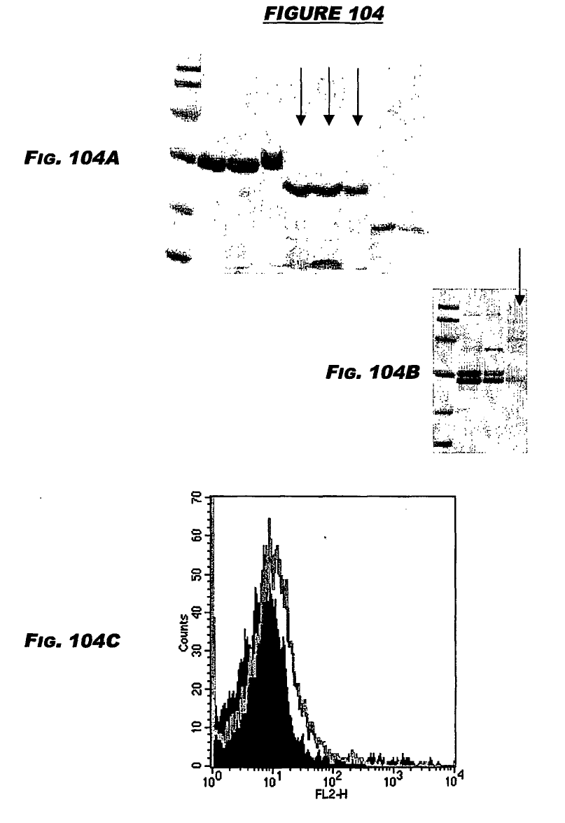

The protein was expressed in E. coli and purified as a GST-fusion product (FIG. 103A) and also in his-tagged form. The recombinant proteins were used to immunize mice, whose sera were used in a Western blot (FIG. 103B) and for FACS analysis (FIG. 103C).

These experiments show that cp6964 is a surface-exposed and immunoaccessible protein, and that it is a useful immunogen. These properties are not evident from the sequence alone.

### Example 104

The following C. pneumoniae protein (PID 4377387) was expressed <SEQ ID 207; cp7387>:

```
    1 LNFAKIDHNH LYLTCLGDLG VACPILSTDC LPNYSEKASH EVLVYSKFRC
  51 ISGEPSRLAT SGNDTYYSIV SLPIGLRYEV TSPSGRHDFN IDMHVAPKIG
101 AVLSHGTREA KEIPGSSKDY AFFSLTARES LMISEKLAMT FQVSEVIQNC
151 YSQCTKVTKT NLKEQYRHLS HNTGFELSVK SAF*
```

The cp7387 nucleotide sequence <SEQ ID 208> is:

```
    1 TTGAATTTTG CAAAGATTGA TCACAATCAT CTCTACCTTA CATGTTTGGG
  51 AGATCTTGGT GTAGCTTGTC CTATACTTTC TACAGATTGT CTACCTAATT
101 ATAGCGAGAA AGCATCTCAT GAGGTTCTTG TTTATAGTAA ATTTAGATGC
151 ATTTCTGGAG AGCCATCTCG ACTTGCAACT TCAGGAAATG ACACATATTA
201 TTCTATAGTA AGTTTACCTA TAGGACTCCG TTACGAAGTG ACTTCACCAT
251 CAGGACGTCA TGATTTCAAT ATTGATATGC ATGTAGCTCC AAAGATAGGT
301 GCAGTACTCT CTCATGGAAC ACGAGAGGCT AAAGAGATCC CAGGATCTTC
351 AAAAGACTAT GCATTTTTTA GCTTGACTGC TAGAGAAAGT TTAATGATTT
401 CTGAAAAGCT TGCGATGACT TTCCAAGTTA GCGAAGTTAT TCAGAATTGT
451 TATTCACAAT GTACTAAAGT AACGAAAACT AATTTAAAAG AACAGTATAG
501 GCACTTATCC CACAATACAG GGTTTGAGTT AAGCGTCAAG TCTGCATTCT
551 AA
```

The PSORT algorithm predicts inner membrane (0.043).

The protein was expressed in E. coli and purified as a his-tagged-fusion product (FIG. 104A) and also as a GST-fusion (FIG. 104B). The recombinant proteins were used to immunize mice, whose sera were used in a Western blot and for FACS analysis (FIG. 104C; his-tagged).

These experiments show that cp7387 is a surface-exposed and immunoaccessible protein, and that it is a useful immunogen. These properties are not evident from the sequence alone.

### Example 105

The following C. pneumoniae protein (PID 4376281) was expressed <SEQ ID 209; cp6281>:

```
    1 MFLQFFHPTV FSDQSLSFLP YLGKSSGIIE KCSNIVEHYL HLGGDTSVII
  51 TGVSGATFLS VDHALPISKS EKIIKILSYI LILPLILALF IKIVLRIILF
101 FKYRGLILDV KKEDLKKTLT PDQENLSLPL PSPTTLKKTH ALHILVRSGK
151 TYNELIQEGE SFTKITDLGQ APSPKQDIGF SYNSLLPNFY FHSLVSVPNI
201 SGEERALNYH KEQQEEMAVK LKTMQACSFV FRSLHLPSMQ TKDKKAGFGL
251 LTFFPWKTYP L*
```

The cp6281 nucleotide sequence <SEQ ID 210> is:

```
    1 ATGTTTCTTC AGTTTTTTCA TCCTATAGTC TTCTCGGATC AGTCCTTATC
  51 TTTTCTTCCT TACCTAGGAA AAAGCTCTGG CATTATTGAA AAATGTTCCA
101 ATATCGTTGA ACACTATTTA CATTTGGGAG GAGACACTTC TGTTATCATC
151 ACAGGAGTTT CTGGAGCTAC CTTTCTATCT GTTGATCATG CCCTCCCAAT
201 CTCGAAATCT GAAAAAATAA TAAAAATTCT CTCCTATATT TTAATTCTTC
251 CTCTGATTCT AGCTCTCTTT ATTAAGATCG TTTTACGCAT TATCTTATTC
301 TTCAAGTATC GTGGTCTAAT CCTAGATGTT AAGAAGGAGG ATTTGAAAAA
351 AACACTTACA CCTGACCAAG AAAACCTCAG TCTTCCTTTA CCATCTCCTA
401 CAACATTAAA GAAAATTCAT GCGCTACACA TTTTAGTGCG TTCTGGAAAA
451 ACCTATAACG AGCTTATACA AGAAGGGTTT TCTTTCACTA AAATCACAGA
501 TCTTGGTCAA GCTCCTTCAC CAAAGCAAGA TATTGGCTTC TCTTATAATT
551 CCCTTCTCCC TAACTTCTAT TTTCATTCCT TGGTATCTGT TCCAAATATT
601 TCAGGCGAGG AACGGGCTCT TAATTATCAT AAAGAACAAC AAGAGGAAAT
651 GGCTGTTAAA TTAAAAACAA TGCAAGCGTG TTCTTTTGTC TTCCGATCCC
701 TGCATTTACC TTCAATGCAA ACGAAGGACA AAAAGGCTGG ATTTGGACTA
751 CTGACGTTTT TCCCTTGGAA AATCTACCCC CTATAA
```

The PSORT algorithm predicts inner membrane (0.5373).

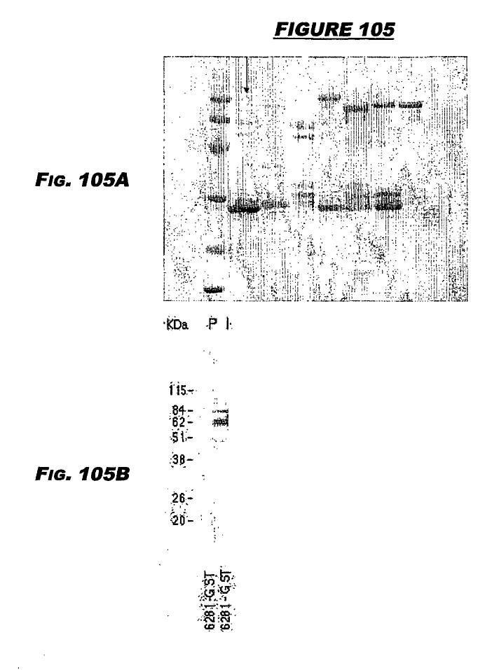

The protein was expressed in E. coli and purified as a GST-fusion product (FIG. 105A). The recombinant protein was used to immunize mice, whose sera were used in a Western blot (FIG. 105B) and for FACS analysis.

These experiments show that cp6281 is a surface-exposed and immunoaccessible protein, and that it is a useful immunogen. These properties are not evident from the sequence alone.

### Example 106 and Example 107

The following C. pneumoniae protein (PID 4376306) was expressed <SEQ ID 211; cp6306>:

1 MGNHETYIHP GVLPSSHAQD VSRSTVYPSR SFIMRRMLMG WNFNRVPSKS
51 SEQLMDGHRI PLIFFGKHHP TISILNVNRF SWLSIFYNGE RGF*

The cp6306 nucleotide sequence <SEQ ID 212> is:

```
    1 ATGGGAAACC ATGAGACCTA TATACATCCA GGAGTGCTCC CGAGTAGTCA
  51 TGCTCAGGAT GTTAGCAGAT CTACAGTTTA CCCCAGTCGA AGTTTTATCA
101 TGAGACGTAT GCTCATGGGC TGGAATTTCA ATCGTGTTCC CTCGAAGAGC
151 TCCGAGCAGT TAATGGATGG TCATCGCATA CCTCTTATAT TTTTTGGGAA
201 GCATCATCCT ACTATATCTA TTTTAAATGT CAATAGATTT TCTTGGCTCT
251 CCATTTTTTA CAATGGAGAA AGGGGGTTTT GA
```

The PSORT algorithm predicts cytoplasm (0.167).

The following C. pneumoniae protein (PID 4376434) was also expressed <SEQ ID 213; cp6434>:

```
    1 MSESINRSIH LEASTPFFIK LTNLCESRLV KITSLVISLL ALVGAGVTLV
  51 VLFVAGILPL LPVLILEIIL ITVLVLLFCL VLEPYLIEKP SKIKELPKVD
101 ELSVVETDST L*
```

The cp6434 nucleotide sequence <SEQ ID 214> is:

```
    1 ATGTCTGAAA GTATTAACAG AAGCATTCAT TTAGAAGCCT CTACACCATT
  51 TTTTATAAAA TTAACGAATC TCTGTGAAAG TAGATTAGTT AAGATCACTT
101 CTCTTGTTAT TTCTCTATTA GCTTTAGTGG GTGCGGGAGT CACTCTTGTG
151 GTTTTATTTG TAGCTGGGAT CCTTCCTTTA CTTCCTGTAC TCATCTTAGA
201 AATTATTTTA ATAACCGTCC TTGTCTTGCT TTTTTGTTTG GTATTGGAAC
251 CTTATTTAAT AGAAAAACCT AGTAAAATAA AGGAACTACC TAAAGTAGAC
301 GAGCTATCTG TAGTAGAAAC GGACAGTACT CTTTAA
```

The PSORT algorithm predicts inner membrane (0.6859).

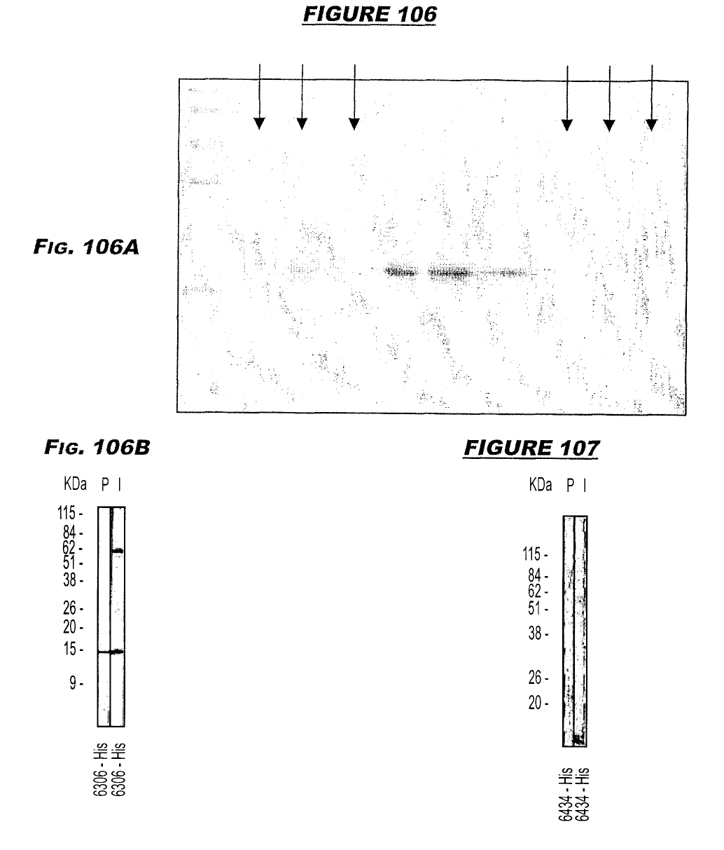

The proteins were expressed in E. coli and purified as his-tag products (FIG. 106A; 6306=lanes 2-4; 6434=lanes 8-10). The recombinant proteins were used to immunize mice, whose sera were used in Western blots (FIGS. 106B & 107) and for FACS analysis.

These experiments show that cp6306 & cp6434 are surface-exposed and immunoaccessible proteins, and that they are useful immunogens. These properties are not evident from the sequences alone.

### Example 108

The following C. pneumoniae protein (PID 4377400) was expressed <SEQ ID 215; cp7400>:

1 MRVMRFFCLF FLGFLGSFHC VAEDKGVDLF GVWDDNQITE CDDSYMTEGR
51 EEVEKVVDA

The cp7400 nucleotide sequence <SEQ ID 216> is:

```
    1 GTGAGAGTTA TGAGATTTTT TTGTCTATTT TTTCTTGGGT TCCTAGGATC
  51 TTTTCATTGT GTTGCTGAAG ACAAGGGCGT GGATTTATTT GGAGTCTGGG
101 ACGATAACCA AATTACAGAG TGTGACGATA GTTACATGAC AGAGGGTCGT
151 GAAGAGGTTG AAAAGGTAGT GGACGCTTAG
```

The PSORT algorithm predicts periplasmic space (0.924).

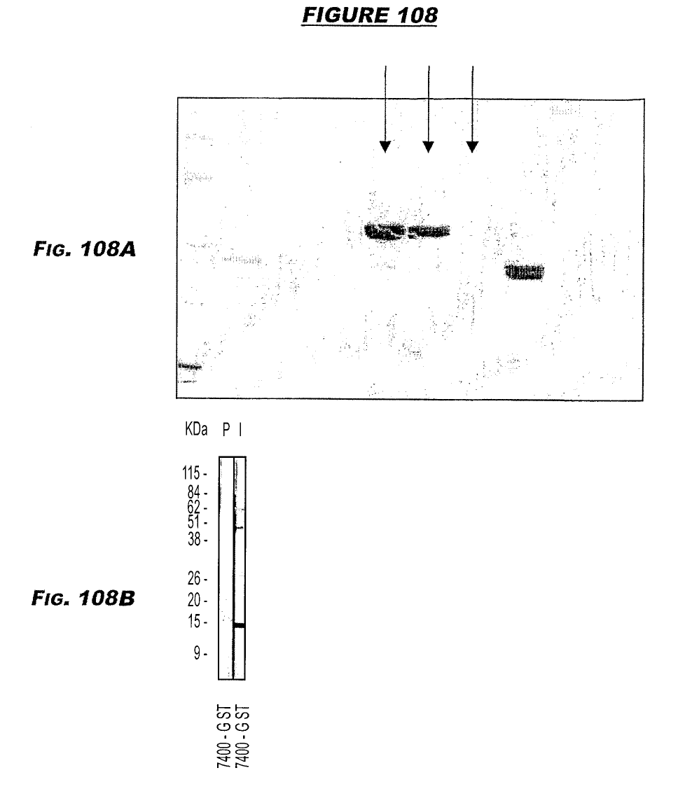

The protein was expressed in E. coli and purified as a GST-fusion product (FIG. 108A). The recombinant protein was used to immunize mice, whose sera were used in a Western blot (FIG. 108B) and for FACS analysis.

These experiments show that cp7400 is a surface-exposed and immunoaccessible protein, and that it is a useful immunogen. These properties are not evident from the sequence alone.

### Example 109

The following C. pneumoniae protein (PID 4376395) was expressed <SEQ ID 217; cp6395>:

| | |
|---|---|
| 1 | MENAMSSSFV YNGPSWILKT SVAQEVFKKH GKGIQVLLST SVMLFIGLGV |
| 51 | CAFIFPQYLI VFVLTIALLM LAISLVLFLL IRSVRSSMVD RLWCSEKGYA |
| 101 | LHQHENGPFL DVKRVQQILL RSPYIKVRAL WPSGDIPEDP SQAAVLLLSP |
| 151 | WTFFSSVDVE ALLPSPQEKE GKYIDPVLPK LSRIERVSLL VFLSAFTLDD |
| 201 | LNEQGVNPLM NNEEFLFFIN KKAREHGIQD LKHEIMSSLE KTGVPLDPSM |
| 251 | SFQVSQAMFS VYRYLRQRDL TTSELRCFHL LSCFKGDVVH CLASFENPKD |
| 301 | LADSDFLEAC KNVEWGEFIS ACEKALLKNP QGISIKDLKQ FLVR* |

The cp6395 nucleotide sequence <SEQ ID 218> is:

| | |
|---|---|
| 1 | ATGGAGAATG CTATGTCATC ATCGTTTGTG TATAATGGGC CTTCGTGGAT |
| 51 | TTTAAAAACG TCAGTAGCTC AGGAGGTATT TAAAAAGCAC GGTAAGGGGA |
| 101 | TTCAGGTTCT CTTAAGTACT TCAGTGATGC TTTTTATAGG TCTTGGAGTC |
| 151 | TGTGCCTTTA TATTTCCTCA ATATCTGATT GTTTTTGTTT TGACTATAGC |
| 201 | TTTGCTTATG CTCGCTATAA GCTTGGTATT GTTTCTCTTA ATACGTTCTG |
| 251 | TACGCTCTTC AATGGTAGAT CGTTTGTGGT GTTCTGAAAA AGGATATGCT |
| 301 | CTTCATCAAC ATGAGAACGG GCCTTTTTTG GATGTGAAGC GTGTACAGCA |
| 351 | AATTCTTCTA AGATCACCCT ATATTAAAGT TCGGGCTTTA TGGCCGTCTG |
| 401 | GAGATATCCC TGAGGATCCT TCACAAGCTG CGGTTCTATT ACTTTCTCCT |
| 451 | TGGACTTTCT TTTCATCCGT GGATGTAGAG GCTTTATTAC CGAGTCCTCA |
| 501 | AGAAAAGGAG GGTAAGTATA TAGATCCTGT GCTGCCTAAG TTGTCTAGGA |
| 551 | TAGAGAGAGT CTCACTTTTA GTGTTTTTGA GTGCATTTAC TTTGGATGAC |
| 601 | TTAAACGAAC AGGGAGTCAA TCCTTTGATG AATAATGAGG AATTTTTATT |
| 651 | TTTTATAAAT AAGAAAGCGC GTGAGCATGG GATTCAGGAT TTAAAACACG |
| 701 | AGATTATGTC TTCGTTAGAG AAAACAGGAG TGCCATTAGA CCCCTCAATG |
| 751 | AGTTTTCAAG TTTCACAAGC GATGTTTTCT GTATATCGCT ACTTGAGACA |
| 801 | AAGGGATTTA ACGACTTCAG AATTAAGATG TTTTCACCTC TTAAGTTGTT |
| 851 | TTAAAGGGGA TGTGGTTCAT TGTTTAGCTT CATTTGAAAA CCCTAAAGAT |
| 901 | TTAGCAGATT CTGACTTTTT AGAAGCTTGT AAGAACGTGG AATGGGGTGA |
| 951 | GTTTATTTCG GCATGTGAGA AGGCTCTTTT AAAGAATCCG CAAGGAATTT |
| 1001 | CCATTAAGGA TCTAAAACAA TTTTTAGTGA GGTAA |

The PSORT algorithm predicts inner membrane (0.6307).

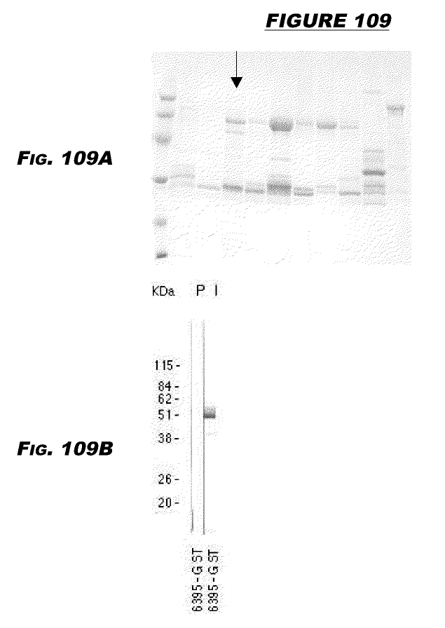

The protein was expressed in E. coli and purified as a GST-fusion product (FIG. 109A). The recombinant protein was used to immunize mice, whose sera were used in a Western blot (FIG. 109B) and for FACS analysis.

These experiments show that cp6395 is a surface-exposed and immunoaccessible protein, and that it is a useful immunogen. These properties are not evident from the sequence alone.

### Example 110

The following C. pneumoniae protein (PID 4376396) was expressed <SEQ ID 219; cp6396>:

| | |
|---|---|
| 1 | MIEFAFVPHT SVTADRIEDR MACRMNKLST LAITSLCVLI SSVCIMIGIL |
| 51 | CISGTVGTYA FVVGIIFSVL ALVACVFFLY FFYFSSEEFK CASSQEFRFL |
| 101 | PIPAVVSALR SYEYISQDAI NDVIKDTMQL STLSSLLDPE AFFLEFPYFN |
| 151 | SLIVNHSMKE ADRLSREAFL ILLGEITWKD CETKILPWLK DPNITPDDFW |
| 201 | KLLKDHFDLK DFKKRIATWI RKAYPEIRLP KKHCLDKSIY KGCCKFLLLS |
| 251 | ENDVQYQRLL HKVCYFSGEF PAMVLGLGSE VPMVLGLPKV PKDLTWEMFM |
| 301 | ENMPVLLQSK REGHWKISLE DVASL* |

The cp6396 nucleotide sequence <SEQ ID 220> is:

| | |
|---|---|
| 1 | ATGATCGAGT TTGCTTTTGT TCCTCATACC TCCGTGACAG CGGATCGGAT |
| 51 | TGAGGATCGC ATGGCCTGTC GCATGAACAA GTTGTCTACT TTAGCAATTA |
| 101 | CAAGTCTTTG TGTATTGATC AGTTCAGTTT GTATTATGAT TGGGATTTTA |
| 151 | TGCATTTCTG GAACGGTTGG GACCTATGCA TTTGTTGTAG GAATTATTTT |
| 201 | TTCTGTGCTT GCTTTGGTAG CATGTGTTTT CTTTCTTTAT TTCTTTTATT |
| 251 | TTTCTTCTGA GGAATTTAAG TGTGCTTCTT CGCAGGAGTT TCGTTTTTTG |
| 301 | CCTATACCAG CTGTGGTTTC TGCATTGCGT TCCTATGAAT ACATTTCTCA |
| 351 | GGACGCTATC AATGACGTTA TAAAAGATAC GATGCAGTTG TCTACCCTTT |
| 401 | CTTCTCTTTT AGATCCCGAA GCTTTTTTCT TAGAATTTCC TTATTTTAAC |
| 451 | TCTTTGATAG TGAATCATTC GATGAAGGAA GCGGATCGTT TGTCTCGAGA |
| 501 | GGCTTTTTTG ATTTTATTAG GTGAGATTAC TTGGAAGGAT TGTGAAACAA |
| 551 | AAATTTTGCC ATGGTTGAAA GATCCTAATA TCACTCCTGA TGATTTCTGG |
| 601 | AAGCTATTAA AAGACCATTT CGATTTAAAG GACTTTAAGA AGAGGATCGC |
| 651 | CACTTGGATA CGGAAGGCCT ATCCAGAAAT TAGATTACCG AAGAAGCATT |
| 701 | GTTTAGATAA GTCTATCTAT AAGGGGTGTT GTAAGTTTTT ATTACTTTCT |
| 751 | GAGAATGATG TGCAATATCA GAGGTTATTA CATAAGGTCT GTTATTTCTC |
| 801 | TGGGGAGTTT CCTGCCATGG TTTTAGGTTT GGGAAGTGAA GTGCCTATGG |
| 851 | TGTTAGGACT CCCTAAGGTT CCCAAGGATC TTACCTGGGA GATGTTTATG |
| 901 | GAAAATATGC CTGTTCTTCT GCAAAGCAAA AGAGAGGGGC ATTGGAAAAT |
| 951 | CTCCTTGGAA GACGTAGCCT CTCTTTAA |

The PSORT algorithm predicts inner membrane (0.6095).

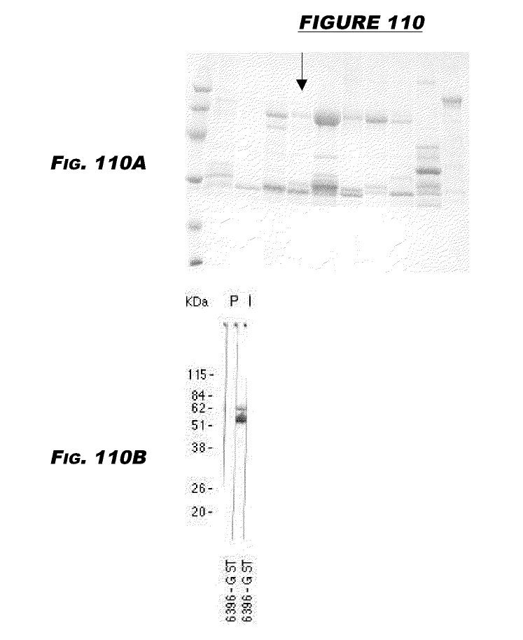

The protein was expressed in E. coli and purified as a GST-fusion product (FIG. 110A). The recombinant protein was used to immunize mice, whose sera were used in a Western blot (FIG. 110B) and for FACS analysis.

These experiments show that cp6396 is a surface-exposed and immunoaccessible protein, and that it is a useful immunogen. These properties are not evident from the sequence alone.

### Example 111

The following C. pneumoniae protein (PID 4376408) was expressed <SEQ ID 221; cp6408>:

| | |
|---|---|
| 1 | MNTSLKRPLK SHFDVVGSFL RPEHLKKTRE SLKEGSISLD QLMQIEDIAI |
| 51 | QDLIKKQKAA GLSFITDGEF RRATWHYDFM WGFHGVGHHR ATEGVFFDGE |
| 101 | RAMIDDTYLT DKISVSHHPF VDHFKFVKAL EDEFTTAKQT LPAPAQFLKQ |
| 151 | MIFPNNIEVT RKFYPTNQEL IEDIVAGYRK VIRDLYDAGC RYLQLDDCTR |
| 201 | GGLVDPRVCS WYGIDEKGLQ DLIQQYLLIN NLVIADRPDD LVVNLHVCRG |
| 251 | NYHSKFFASG SYDFIAKPLF EQTNVDGYYL EFDHERSGDF SPLTFISGEK |
| 301 | TVCLGLVTSK TPTLENKDEV IARIHQAADY LPLERLSLSP QCGFASCEIG |
| 351 | NKLTEEEQWA KVALVKEISE EVWK* |

The cp6408 nucleotide sequence <SEQ ID 222> is:

| | |
|---|---|
| 1 | ATGAATACTT CACTAAAAAG ACCTCTGAAA TCTCATTTTG ATGTTGTCGG |
| 51 | TAGTTTTTTG CGTCCTGAGC ATTTAAAAAA AACTAGAGAA AGCCTTAAAG |
| 101 | AAGGCTCTAT TTCTCTAGAT CAACTCATGC AAATTGAGGA TATCGCTATC |
| 151 | CAAGATTTGA TCAAAAAACA AAAAGCAGCA GGTCTTTCTT TTATTACTGA |
| 201 | TGGAGAATTC CGCAGAGCTA CGTGGCATTA CGACTTCATG TGGGGTTTTC |
| 251 | ATGGCGTAGG TCACCACAGA GCTACAGAAG GAGTTTTCTT TGATGGAGAA |
| 301 | CGCGCTATGA TCGATGATAC CTATCTGACA GACAAGATCT CTGTATCTCA |
| 351 | CCACCCATTT GTGGATCACT TTAAATTTGT AAAAGCTCTA GAAGATGAAT |
| 401 | TTACGACTGC AAAGCAAACT CTTCCTGCAC CGGCACAGTT TTTAAAGCAG |
| 451 | ATGATCTTCC CTAATAATAT AGAGGTCACA CGTAAATTCT ATCCTACAAA |
| 501 | TCAGGAGCTA ATTGAAGATA TTGTTGCAGG TTATCGTAAA GTCATTCGCG |
| 551 | ATCTTTATGA TGCTGGCTGC CGCTATCTCC AATTAGATGA CTGTACTCGG |
| 601 | GGAGGTTTAG TAGACCCTCG AGTCTGTTCG TGGTATGGTA TCGATGAAAA |
| 651 | AGGTCTTCAA GATCTGATTC AACAATATCT TCTGATTAAT AATCTTGTAA |
| 701 | TTGCAGATCG TCCCGATGAT CTAGTCGTTA ATTTACATGT ATGCCGTGGG |
| 751 | AACTACCACT CAAAATTCTT TGCTAGTGGT AGTTATGACT TTATTGCAAA |
| 801 | GCCCCTATTC GAACAAACAA ATGTAGACGG CTACTATTTA GAGTTTGATC |
| 851 | ATGAGCGTTC TGGAGACTTC TCTCCTCTCA CCTTCATTTC TGGAGAAAAA |
| 901 | ACTGTCTGCT TAGGTCTTGT TACCAGCAAA ACCCCTACAC TTGAAAATAA |
| 951 | GGATGAGGTC ATTGCTCGCA TACATCAAGC AGCAGACTAC CTGCCCTTGG |
| 1001 | AAAGACTCTC TCTAAGTCCA CAGTGTGGTT TTGCTTCATG TGAAATAGGA |
| 1051 | AATAAATTAA CAGAAGAAGA GCAATGGGCT AAAGTTGCTC TAGTAAAAGA |
| 1101 | AATTTCCGAA GAAGTTTGGA AATAA |

The PSORT algorithm predicts cytoplasm (0.2171).

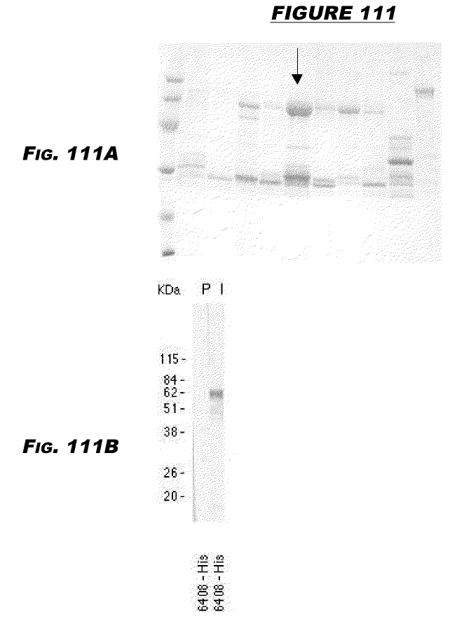

The protein was expressed in E. coli and purified as a GST-fusion product (FIG. 111A) and also as a his-tagged product. The his-tag protein was used to immunize mice, whose sera were used in a Western blot (FIG. 111B) and for FACS analysis.

These experiments show that cp6408 is a surface-exposed and immunoaccessible protein, and that it is a useful immunogen. These properties are not evident from the sequence alone.

### Example 112

The following C. pneumoniae protein (PID 4376430) was expressed <SEQ ID 223; cp6430>:

| | |
|---|---|
| 1 | MKLYSISSDV DTPWIFQLMS KVDSYLFLGG NRIKVVSIVM QEPNLIIGKV |
| 51 | ENVRISTIVK ILKILSFLIF PLILIALALH YFLHAKYANH LLVSKILERA |
| 101 | PQYVPIPGRS GDTASHYKLT TLVPVSQKNL QAMGSNPLEV EAALRTTKPS |
| 151 | FFCVPAKYRQ IIISSHGIRF SLDLEQLADD INLDSVSWPT EYLNSTMDFC |
| 201 | SKADKRVIQN VQNLRTGTYI NSVGKRSLLK FMLQHLFIDG ITQENPEALP |
| 251 | NNTSGRLTLF PSVRYIYSHF TPQNPTIWPQ VFFRQGPLDE DRGGGFEILE |
| 301 | QLQELGVRFP ICPSQGPDNP NFQGFQGIRI YWEDSYQPNK EV* |

The cp6430 nucleotide sequence <SEQ ID 224> is:

| | |
|---|---|
| 1 | ATGAAACTTT ATAGCATCTC TTCAGATGTA GATACACCTT GGATATTTCA |
| 51 | GCTTATGTCA AAGGTAGATT CTTATCTTTT CTTAGGCGGG AATAGAATCA |
| 101 | AGGTTGTATC TATAGTTATG CAAGAACCTA ACTTAATTAT TGGAAAAGTA |
| 151 | GAAAACGTTC GGATCTCCAC AATAGTGAAA ATATTAAAGA TTTTATCCTT |
| 201 | CTTAATCTTC CCTCTGATTT TAATCGCTTT AGCCCTACAC TATTTTCTAC |
| 251 | ATGCTAAATA TGCTAATCAC TTACTTGTAT CTAAGATTTT AGAAAGAGCT |
| 301 | CCTCAGTATG TGCCTATTCC TGGTCGTTCA GGAGACACGG CGTCTCATTA |
| 351 | TAAATTAACA ACATTGGTTC CAGTATCCCA AAAAAATCTA CAAGCTATGG |
| 401 | GATCAAATCC TCTAGAAGTT GAAGCGGCTC TTCGAACTAC AAAACCCTCT |
| 451 | TTTTTCTGTG TACCTGCAAA ATACCGTCAG ATTATAATTT CAAGTCACGG |
| 501 | CATTCGCTTT TCTTTAGATC TTGAACAACT TGCTGATGAC ATTAATTTAG |
| 551 | ATTCGGTTTC CTGGCCTACG GAGTATCTTA ACTCTACTAT GGATTTTTGC |
| 601 | AGCAAGGCAG ATAAACGTGT TATACAGAAT GTACAAAATC TGCGGACAGG |
| 651 | AACTTACATA AATTCTGTAG GAAAGCGTAG CCTTTTAAAA TTCATGTTAC |
| 701 | AGCACCTATT TATTGATGGG ATCACACAAG AAAACCCTGA AGCCCTTCCT |
| 751 | AACAATACAT CTGGAAGACT GACTCTATTC CCTAGTGTTC GTTATATCTA |
| 801 | TTCTCATTTT ACTCCACAAA ATCCTACAAT ATGGCCGCAA GTCTTTTTCA |
| 851 | GACAAGGTCC TCTAGATGAA GATCGAGGAG GAGGATTTGA GATCTTAGAG |
| 901 | CAATTACAAG AGTTAGGAGT TAGGTTTCCA ATTTGCCCCT CTCAAGGACC |
| 951 | AGACAATCCT AATTTTCAAG GTTTTCAAGG GATTCGTATC TATTGGGAAG |
| 1001 | ATTCCTATCA ACCCAATAAG GAGGTTTAA |

The PSORT algorithm predicts inner membrane (0.5140).

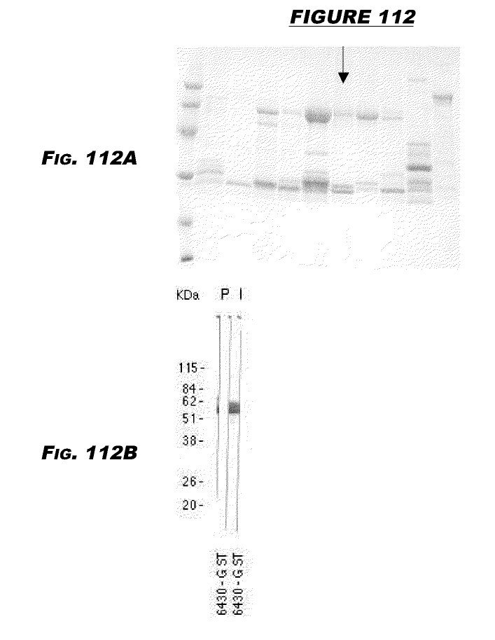

The protein was expressed in E. coli and purified as a GST-fusion product (FIG. 112A). The recombinant protein was used to immunize mice, whose sera were used in a Western blot (FIG. 112B) and for FACS analysis.

These experiments show that cp6430 is a surface-exposed and immunoaccessible protein, and that it is a useful immunogen. These properties are not evident from the sequence alone.

### Example 113

The following C. pneumoniae protein (PID 4376439) was expressed <SEQ ID 225; cp6439>:

| | |
|---|---|
| 1 | MSYDTLFKNL EKEDSVHKIC NEIFALVPRL NTIACTEAII KNLPKADIHV |
| 51 | HLPGTITPQL AWILGVKNGF LKWSYNSWTN HRLLSPKNPH KQYSNIFRNF |
| 101 | QDICHEKDPD LSVLQYNILN YDFNSFDRVM ATVQGHRFPP GGIQNEEDLL |
| 151 | LIFNNYLQQC LDDTIVYTEV QQNIRLAHVL YPSLPEKHAR MKFYQILYRA |
| 201 | SQTFSKHGIT LRFLNCFNKT FAPQINTQEP AQEAVQWLQE VDSTFPGLFV |
| 251 | GIQSAGSESA PGACPKRLAS GYRNAYDSGF GCEAHAGEGI ETRTIFSSAK |
| 301 | VNPEGLIEIT RVTFSSLKRK QPSSLPIRVT CQLG* |

The cp6439 nucleotide sequence <SEQ ID 226> is:

| | |
|---|---|
| 1 | ATGTCTTATG ATACGTTATT CAAGAATCTT GAAAAGGAAG ATTCTGTACA |
| 51 | TAAGATATGC AATGAGATCT TTGCATTAGT ACCACGACTC AATACAATCG |
| 101 | CTTGCACCGA AGCTATCATC AAAAACCTCC CCAAAGCAGA TATCCATGTA |
| 151 | CACCTTCCTG GGACCATAAC ACCTCAATTA GCTTGGATTT TAGGTGTGAA |
| 201 | AAATGGGTTC TTAAAATGGT CTTATAATTC TTGGACCAAT CATCGATTAC |
| 251 | TTTCTCCTAA GAATCCTCAT AAACAATACT CCAATATTTT CCGAAACTTT |
| 301 | CAAGATATCT GTCACGAAAA GGATCCGGAT TTAAGTGTAT TACAATATAA |
| 351 | TATCTTAAAT TACGATTTTA ATAGCTTTGA TAGAGTGATG GCTACAGTAC |
| 401 | AAGGACATCG CTTTCCTCCT GGAGGAATCC AAAATGAAGA AGACCTTCTT |
| 451 | CTCATTTTCA ATAACTATCT CCAGCAATGT CTGGACGATA CTATCGTGTA |
| 501 | TACTGAAGTA CAACAAAATA TCCGCCTTGC CCATGTTTTG TATCCTTCAT |
| 551 | TACCTGAAAA GCACGCGCGT ATGAAGTTTT ATCAAATCTT GTATCGTGCT |
| 601 | TCGCAAACGT TTTCAAAACA CGGGATTACT TTACGATTTT TAAACTGCTT |
| 651 | CAATAAAACA TTTGCTCCAC AAATAAACAC ACAAGAACCT GCCCAAGAAG |
| 701 | CTGTTCAATG GCTCCAAGAG GTTGATTCTA CATTTCCTGG TCTATTTGTA |
| 751 | GGGATACAAT CCGCAGGATC AGAATCTGCG CCCGGAGCCT GTCCTAAGCG |
| 801 | ATTAGCTTCT GGATATAGAA ATGCTTATGA CTCAGGGTTT GGTTGTGAAG |
| 851 | CTCATGCTGG AGAAGGCATA GAGACCCGGA CTATTTTTTC GTCAGCTAAG |
| 901 | GTAAATCCAG AGGGATTGAT CGAGATAACC CGAGTGACTT TCTCGTCTCT |
| 951 | TAAACGAAAA CAGCCATCTA GTTTACCCAT AAGAGTTACT TGCCAGTTAG |
| 1001 | GATAA |

The PSORT algorithm predicts cytoplasm (0.1628).

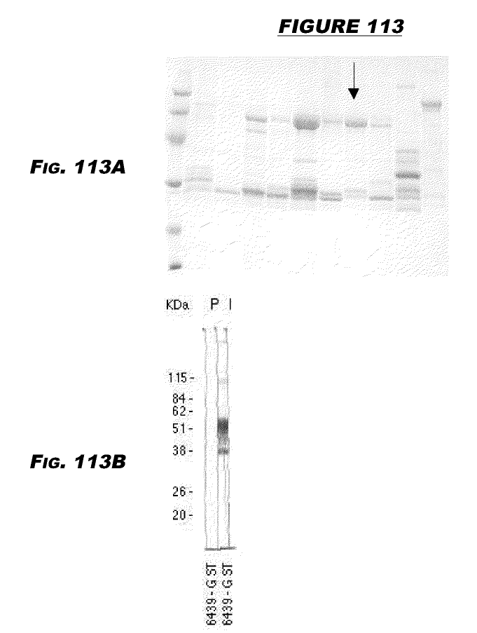

The protein was expressed in E. coli and purified as a GST-fusion product (FIG. 113A). The recombinant protein was used to immunize mice, whose sera were used in a Western blot (FIG. 113B) and for FACS analysis.

These experiments show that cp6439 is a surface-exposed and immunoaccessible protein, and that it is a useful immunogen. These properties are not evident from the sequence alone.

### Example 114

The following C. pneumoniae protein (PID 4376440) was expressed <SEQ ID 227; cp6440>:

| | |
|---|---|
| 1 | LQSARRHLNT IFILDFGSQY TYVLAKQVRK LFVYCEVLPW NISVQCLKER |
| 51 | APLGIILSGG PHSVYENKAP HLDPEIYKLG IPILAICYGM QLMARDFGGT |
| 101 | VSPGVGEFGY TPIHLYPCEL FKHIVDCESL DTEIRMSHRD HVTTTPEGFN |
| 151 | VIASTSQCSI SGIENTKQRL YGLQFHPEVS DSTPTGNKIL ETFVQEICSA |
| 201 | PTLWNPLYIQ QDLVSKIQDT VIEVFDEVAQ SLDVQWLAQG TIYSDVIESS |
| 251 | RSGHASEVIK SHHNVGGLPK NLKLKLVEPL RYLFKDEVRI LGEALGLSSY |
| 301 | LLDRHPFPGP GLTIRVIGEI LPEYLAILRR ADLIFIEELR KAKLYDKISQ |
| 351 | AFALFLPIKS VSVKGDCRSY GYTIALRAVE STDFMTGRWA YLPCDVLSSC |
| 401 | SSRIINEIPE VSRVVYDISD KPPATIEWE* |

The cp6440 nucleotide sequence <SEQ ID 228> is:

| | |
|---|---|
| 1 | TTGCAGAGTG CAAGGAGACA TTTGAACACC ATATTTATTC TAGATTTTGG |
| 51 | ATCTCAATAT ACTTATGTAT TAGCAAAGCA AGTGCGGAAG TTATTTGTAT |
| 101 | ATTGCGAAGT TCTTCCCTGG AATATCTCTG TGCAATGTTT AAAAGAAAGA |
| 151 | GCGCCTTTGG GGATCATTCT CTCAGGAGGT CCTCACTCTG TCTATGAAAA |
| 201 | CAAGGCTCCA CATTTAGATC CTGAAATCTA TAAACTTGGC ATTCCAATTC |
| 251 | TAGCTATTTG CTATGGCATG CAGCTTATGG CTAGAGATTT TGGAGGGACT |
| 301 | GTAAGCCCTG GTGTAGGAGA ATTTGGATAT ACGCCCATCC ATCTGTATCC |
| 351 | TTGTGAGCTC TTCAAACACA TCGTCGACTG CGAATCTCTA GACACAGAGA |
| 401 | TTCGGATGAG CCATCGGGAT CATGTTACGA CAATTCCTGA AGGATTTAAT |
| 451 | GTAATCGCAT CCACCTCACA ATGCTCGATC TCAGGAATAG AAAATACCAA |
| 501 | ACAACGGTTG TACGGGCTGC AATTTCATCC CGAGGTTTCT GACTCCACTC |
| 551 | CAACGGGAAA TAAGATTCTA GAAACTTTTG TTCAAGAGAT CTGTTCTGCT |
| 601 | CCCACACTAT GGAATCCCTT GTATATTCAG CAAGACCTTG TAAGTAAAAT |
| 651 | TCAAGATACC GTTATTGAAG TATTTGATGA AGTCGCTCAG TCATTAGACG |
| 701 | TACAATGGTT AGCTCAAGGA ACCATCTACT CAGATGTTAT TGAGTCCTCA |
| 751 | CGCTCTGGAC ATGCCTCCGA AGTAATAAAA TCACATCATA ATGTAGGGGG |
| 801 | GCTTCCAAAA AATCTTAAGC TGAAGTTAGT CGAGCCCTTA CGTTATTTAT |
| 851 | TTAAAGATGA AGTTCGAATT TTAGGAGAAG CCCTAGGACT TTCTAGCTAT |
| 901 | CTCTTGGACA GGCATCCTTT TCCTGGACCT GGCTTGACAA TTCGTGTGAT |
| 951 | TGGAGAGATC CTTCCTGAAT ATCTAGCCAT TTTACGACGG GCGGACCTCA |
| 1001 | TCTTTATAGA AGAGCTTAGG AAAGCAAAAC TCTACGATAA AATAAGCCAA |
| 1051 | GCCTTTGCTC TATTTCTTCC TATAAAATCA GTATCTGTAA AAGGAGATTG |
| 1101 | TAGAAGCTAT GGTTATACCA TAGCATTACG TGCTGTAGAA TCTACAGATT |
| 1151 | TCATGACAGG ACGATGGGCC TACCTTCCAT GCGATGTTCT CAGTTCTTGC |
| 1201 | TCATCGCGAA TTATTAATGA AATACCCGAG GTAAGCCGAG TGGTCTATGA |
| 1251 | TATTTCTGAC AAGCCACCAG CAACTATAGA ATGGGAATAG |

The PSORT algorithm predicts cytoplasm (0.0481).

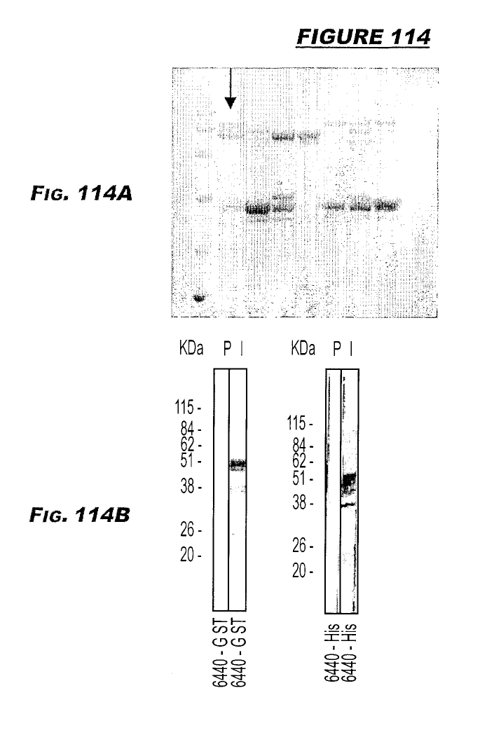

The protein was expressed in E. coli and purified as a GST-fusion product (FIG. 114A) and also as a his-tagged product. The recombinant proteins were used to immunize mice, whose sera were used in a Western blot (FIG. 114B) and for FACS analysis.

These experiments show that cp6440 is a surface-exposed and immunoaccessible protein, and that it is a useful immunogen. These properties are not evident from the sequence alone.

### Example 115

The following C. pneumoniae protein (PID 4376475) was expressed <SEQ ID 229; cp6475>:

| | |
|---|---|
| 1 | MNTYTFSPTL QKSFSLFLLE KLDSYFFFGG TRTQILVITP TNIRLAAKKR |
| 51 | GCKVSTIEKI IKILSFILLP LVIIAFILRY FLHKKFDKQF LCIPKVISNE |
| 101 | DEALLGSRPQ AVEKAVREIS PAFFSIPRKY QLIRIDTPKD DAPSILEPIG |
| 151 | IEIILKDLCI DTLKQSNLFL KREMDFLGHP EEKALFDSIC SIEKDQEWMS |
| 201 | LESKKLLITH FLKYLFVSGI EQLNPGFNPE NGRGYFSEIS TAKIHFHQHG |
| 251 | RYGPIRSSGP IMKEI* |

The cp6475 nucleotide sequence <SEQ ID 230> is:

| | |
|---|---|
| 1 | ATGAATACCT ATACCTTCTC TCCTACACTT CAGAAAAGCT TCAGCCTATT |
| 51 | TCTTTTAGAA AAATTAGACT CTTACTTTTT CTTTGGAGGG ACTCGTACAC |
| 101 | AAATCTTAGT CATCACACCA ACCAATATTA GATTAGCAGC TAAAAAAAGA |
| 151 | GGGTGTAAGG TTTCTACTAT AGAAAAGATA ATCAAGATCC TCTCTTTTAT |
| 201 | CCTGCTGCCC CTAGTTATCA TTGCCTTTAT ACTTCGCTAT TTCTTACATA |
| 251 | AGAAATTCGA TAAACAGTTC TTGTGTATCC CAAAAGTCAT TTCTAACGAA |
| 301 | GACGAAGCTC TTCTTGGATC TAGACCACAA GCAGTTGAAA AAGCAGTTCG |
| 351 | AGAAATATCT CCAGCCTTCT TCTCTATACC AAGAAAATAC CAACTTATTA |
| 401 | GAATCGACAC TCCTAAAGAT GACGCTCCCT CAATCCTTTT CCCTATAGGC |
| 451 | ATAGAGATCA TTCTCAAAGA TTTATGTATT GATACACTCA AGCAATCTAA |
| 501 | TCTTTTCCTT AAAAGAGAAA TGGATTTCTT AGGTCATCCA GAAGAAAAAG |
| 551 | CATTATTCGA CTCGATATGT TCTATAGAAA AAGATCAAGA ATGGATGAGC |
| 601 | TTGGAAAGTA AAAAACTTTT AATCACGCAC TTCCTAAAGT ATCTCTTTGT |
| 651 | CTCTGGAATC GAACAACTAA ATCCAGGCTT TAACCCAGAG AATGGGCGTG |
| 701 | GGTATTTTTC AGAAATAAGT ACAGCAAAGA TCCATTTTCA TCAGCACGGT |
| 751 | CGATATGGGC CAATCCGTTC TTCGGGACCC ATCATGAAGG AAATATAA |

The PSORT algorithm predicts inner membrane (0.5373).

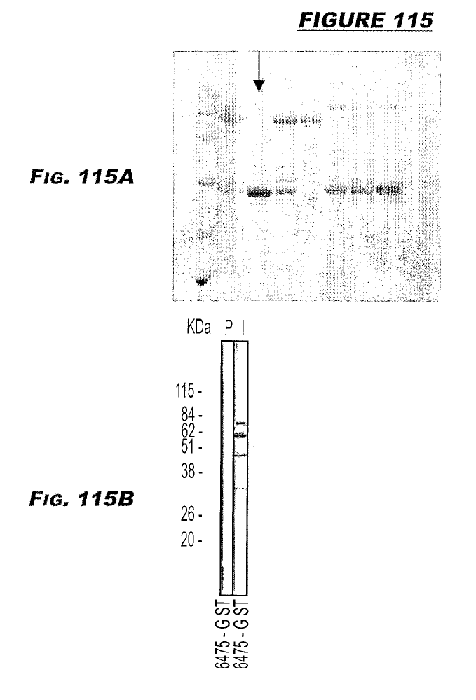

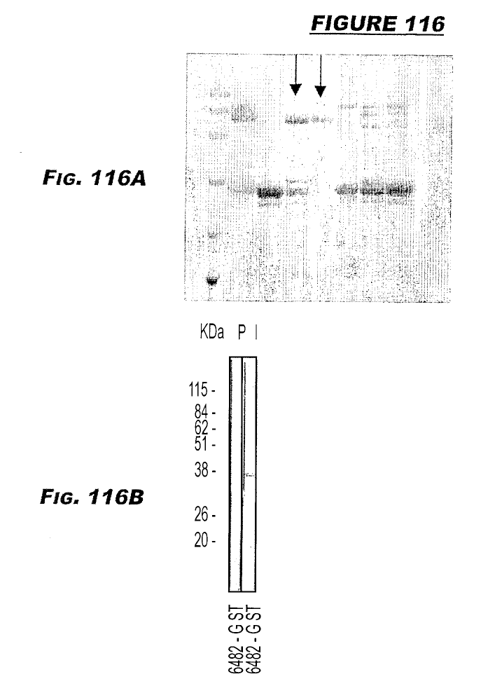

The protein was expressed in E. coli and purified as a GST-fusion product (FIG. 115A). The recombinant protein was used to immunize mice, whose sera were used in a Western blot (FIG. 115B) and for FACS analysis.

These experiments show that cp6475 is a surface-exposed and immunoaccessible protein, and that it is a useful immunogen. These properties are not evident from the sequence alone.

### Example 116

The following C. pneumoniae protein (PID 4376482) was expressed <SEQ ID 231; cp6482>:

| | |
|---|---|
| 1 | MLVELEALKR EFAHLKDQKP TSDQEITSLY QCLDHLEFVL LGLGQDKFLK |
| 51 | ATEDEDVLFE SQKAIDAWNA LLTKARDVLG LGDIGAIYQT IEFLGAYLSK |
| 101 | VNRPAFCIAS EIHFLKTAIR DLNAYYLLDF RWPLCKIEEF VDWGNDCVEI |
| 151 | AKRKLCTFEK ETKELNESLL REEHAMEKCS IQDLQRKLSD IIIELHDVSL |
| 201 | FCFSKTPSQE EYQKDCLYQS RLRYLLLLYE YTLLCKTSTD EQEQAPAKEE |
| 251 | FIREKFSLLE LEKGIKQTKE LEFAIAKSKL ERGCLVMRKY EAAAKHSLDS |
| 301 | MFEEETVKSP RKDTE* |

The cp6482 nucleotide sequence <SEQ ID 232> is:

| | |
|---|---|
| 1 | ATGCTAGTAG AGTTAGAGGC TCTTAAAAGA GAGTTTGCGC ATTTAAAAGA |
| 51 | CCAGAAGCCG ACAAGTGACC AAGAGATCAC TTCACTTTAT CAATGTTTGG |
| 101 | ATCATCTTGA ATTCGTTTTA CTCGGGCTGG GCCAGGACAA ATTTTTAAAG |
| 151 | GCTACGGAAG ATGAAGATGT GCTTTTTGAG TCTCAAAAAG CAATCGATGC |
| 201 | GTGGAATGCT TTATTGACAA AAGCCAGAGA TGTTTTAGGT CTTGGGGACA |
| 251 | TAGGTGCTAT CTATCAGACT ATAGAATTCT TGGGTGCCTA TTTATCAAAA |
| 301 | GTGAATCGGA GGGCTTTTTG TATTGCTTCG GAGATACATT TTCTAAAAAC |
| 351 | AGCAATCCGA GATTTGAATG CATATTACCT GTTAGATTTT AGATGGCCTC |
| 401 | TTTGCAAGAT AGAAGAGTTT GTGGATTGGG GGAATGATTG TGTTGAAATA |
| 451 | GCAAAGAGGA AGCTATGCAC TTTTGAAAAA GAAACCAAGG AGCTCAATGA |
| 501 | GAGCCTTCTT AGAGAGGAGC ATGCGATGGA GAAATGCTCG ATTCAAGATC |
| 551 | TGCAAAGGAA ACTTAGCGAC ATTATTATTG AATTGCATGA TGTTTCTCTT |
| 601 | TTTTGTTTTT CTAAGACTCC CAGTCAAGAG GAGTATCAAA AGGATTGTTT |
| 651 | GTATCAATCA CGATTGAGGT ACTTATTGTT GCTGTATGAG TATACATTGT |
| 701 | TATGTAAGAC ATCCACAGAT TTTCAAGAGC AGGCTAGGGC TAAAGAGGAG |
| 751 | TTCATTAGGG AGAAATTCAG CCTTCTAGAG CTCGAAAAGG GAATAAAACA |
| 801 | AACTAAAGAG CTTGAGTTTG CAATTGCTAA AAGTAAGTTA GAACGGGGCT |
| 851 | GTTTAGTTAT GAGGAAGTAT GAAGCTGCCG CTAAACATAG TTTAGATTCT |
| 901 | ATGTTCGAAG AAGAAACTGT GAAGTCGCCG CGGAAAGACA CAGAATAA |

The PSORT algorithm predicts cytoplasm (0.4607).

The protein was expressed in E. coli and purified as a GST-fusion product (FIG. 116A). The recombinant protein was used to immunize mice, whose sera were used in a Western blot (FIG. 116B) and for FACS analysis.

These experiments show that cp6482 is a surface-exposed and immunoaccessible protein, and that it is a useful immunogen. These properties are not evident from the sequence alone.

### Example 117

The following C. pneumoniae protein (PID 4376486) was expressed <SEQ ID 233; cp6486>:

| | |
|---|---|
| 1 | VVVVALFILG IFFLSGSLAF LVHTSCGVLL GAALPILCIG LVLLAVALIV |
| 51 | FLCHKHKTRQ DLDYYDQDLD SLVIHKKEIP NDISELRVTF EKLQNLFQFH |
| 101 | TKDFSDLSQE LQGKFINCME KWLTLEDEVT KFLIVRDRFL ETRRNFTTFG |
| 151 | EQVKGIQSNI FDLHEEKSSL YLELYRLRKD LQVLLNFFLL PPGILKVDYD |
| 201 | EIEAIKGLFI RLTSRLDKLD VKAQERKKFI NEMSREFKEV EKAFDIVDRA |
| 251 | TKKLMDRAKK ESPARLFMGR TESLLEMKKN EEALKNQGLD PENLSHPELF |
| 301 | SPYQQLLILN YLNSEIVLHH YEFLISGTVT SGLTLEECEN RMRAASTGLN |
| 351 | ALLVRKLQFR GAIKSAYFEK LTEIEKELRS LQDVIKSLEL ELIHKIKDIV |
| 401 | TEET* |

The cp6486 nucleotide sequence <SEQ ID 234> is:

| | |
|---|---|
| 1 | GTGGTGGTTG TCGCTTTATT TATCCTTGGG ATTTTCTTTT TATCTGGTTC |
| 51 | TCTTGCATTC CTTGTTCATA CGTCTTGCGG AGTTCTTTTA GGAGCGGCGC |
| 101 | TTCCCATACT TTGCATAGGT CTTGTTTTAT TGGCTGTAGC TCTTATTGTT |
| 151 | TTCTTATGTC ACAAACACAA GACTCGTCAA GATTTAGATT ATTATGATCA |
| 201 | AGATTTAGAT TCTTTGGTGA TTCATAAGAA AGAGATCCCC AATGACATCT |
| 251 | CTGAGTTGCG GGTAACATTT GAAAAGTTGC AAAATCTGTT TCAGTTCCAT |
| 301 | ACGAAAGATT TCTCTGATCT AAGCCAAGAG CTTCAGGGTA AATTTATCAA |
| 351 | TTGCATGGAG AAATGGCTAA CTTTAGAAGA CGAAGTGACT AAATTTCTTA |
| 401 | TTGTTCGAGA TAGATTTTTA GAAACCAGAA GAAATTTTAC CACTTTTGGA |
| 451 | GAACAGGTTA AAGGGATCCA AAGCAATATT TTTGATTTGC ATGAGGAAAA |
| 501 | GTCTTCATTA TATTTAGAAT TGTATAGGCT TAGGAAAGAC CTCCAAGTTC |
| 551 | TATTAAATTT TTTTCTGCTC CCCCCAGGTA TACTCAAGGT AGATTATGAT |
| 601 | GAAATTGAGG CTATCAAAGG TCTGTTTATA AGATTAACCT CTAGATTAGA |
| 651 | TAAGCTTGAT GTGAAAGCTC AGGAACGTAA GAAGTTCATT AATGAAATGA |
| 701 | GTAGGGAATT TAAAGAAGTA GAGAAAGCTT TTGATATTGT CGATAGGGCA |
| 751 | ACAAAAAAGC TTATGGATAG AGCCAAGAAA GAAAGTCCGG CACGTCTTTT |
| 801 | CATGGGTAGA ACTGAGTCTC TCTTAGAAAT GAAAAAAAAT GAAGAAGCCC |
| 851 | TTAAAAATCA GGGGCTAGAT CCTGAAAATC TTTCCCATCC TGAACTTTTT |
| 901 | AGTCCGTATC AACAGCTTTT AATTTTGAAT TATTTAAATA GCGAAATAGT |
| 951 | TCTGCATCAT TATGAGTTCC TTATTTCTGG AACAGTAACT TCTGGCCTAA |
| 1001 | CTCTTGAAGA ATGTGAAAAT CGAATGAGGG CGGCTTCTAC TGGGTTGAAC |
| 1051 | GCCCTTCTGG TGCGTAAGCT CCAGTTCAGA GGTGCTATAA AATCTGCGTA |
| 1101 | TTTTGAAAAA CTCACAGAGA TTGAAAAAGA GTTACGATCA CTTCAAGACG |
| 1151 | TAATAAAGTC ATTGGAACTA GAACTGATCC ATAAGATAAA AGATATAGTG |
| 1201 | ACAGAAGAAA CTTAG |

The PSORT algorithm predicts inner membrane (0.7474).

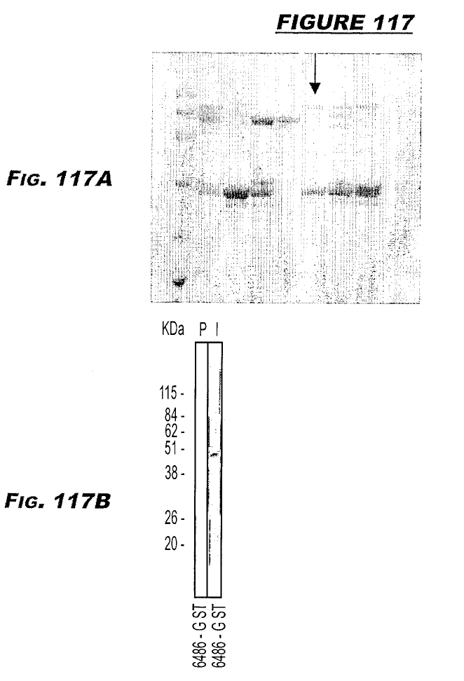

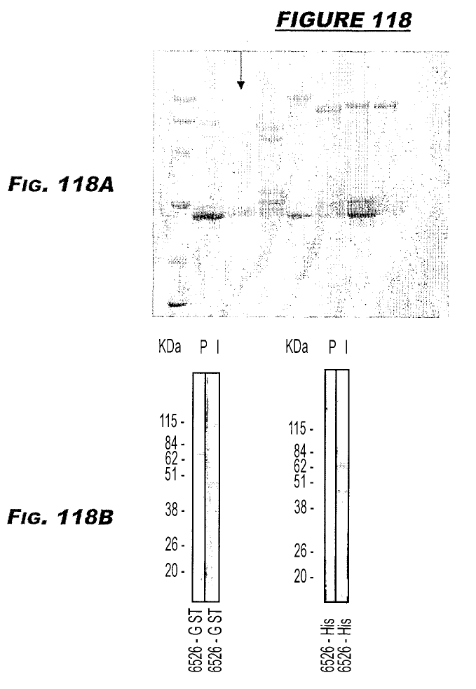

The protein was expressed in E. coli and purified as a GST-fusion product (FIG. 117A). The recombinant protein was used to immunize mice, whose sera were used in a Western blot (FIG. 117B) and for FACS analysis.

These experiments show that cp6486 is a surface-exposed and immunoaccessible protein, and that it is a useful immunogen. These properties are not evident from the sequence alone.

### Example 118

The following C. pneumoniae protein (PID 4376526) was expressed <SEQ ID 235; cp6526>:

| | |
|---|---|
| 1 | MSPFKKIVNR LLCYISFQKE SRTLPIIIRE PRMTTKSLGS FNSVISKNKI |
| 51 | HFISLGCSRN LVDSEVMLGI LLKAGYESTN EIEDADYLIL NTCAFLKSAR |
| 101 | DEAKDYLDHL IDVKKENAKI IVTGCMTSNH KDELKPWMSH IHYLLGSGDV |
| 151 | ENILSAIESR ESGEKISAKS YIEMGEVPRQ LSTPKHYAYL KVAEGCRKRC |
| 201 | AFCIIPSIKG KLRSKPLDQI LKEFRILVNK SVKEIILIAQ DLGDYGKDLS |
| 251 | TDRSSQLESL LHELLKEPGD YWLRMLYLYP DEVSDGIIDL MQSNPKLLPY |
| 301 | VDIPLQHIND RILKQMRRTT SREQILGFLE KLPAKVPQVY IRSSVIVGFP |
| 351 | GETQEEFQEL ADFIGEGWID NLGIFLYSQE ANTPAAELPD QIPEKVKESR |
| 401 | LKILSQIQKR NVDKHNQKLI GEKIEAVIDN YHPETNLLLT ARFYGQAPEV |
| 451 | DPCIIVNEAK LVSHFGERCF IEITGTAGYD LVGRVVKKSQ NQALLKTSKA |
| 501 | * |

The cp6526 nucleotide sequence <SEQ ID 236> is:

| | |
|---|---|
| 1 | ATGAGTCCTT TTAAGAAAAT AGTAAATCGC TTACTATGCT ATATTTCTTT |
| 51 | TCAAAAAGAA TCAAGAACTC TCCCAATCAT TATTAGAGAA CCTAGGATGA |
| 101 | CAACAAAAAG TTTAGGATCT TTCAATTCAG TTATTTCCAA AAATAAAATT |
| 151 | CATTTTATTA GTTTGGGATG CTCTCGGAAC CTTGTAGATA GCGAAGTCAT |
| 201 | GCTAGGCATT CTTCTTAAGG CAGGTTACGA GTCTACTAAT GAAATTGAAG |
| 251 | ATGCTGACTA TTTAATTTTA AATACCTGTG CGTTTTTAAA AAGTGCTAGA |
| 301 | GATGAAGCTA AAGATTATCT AGACCATCTA ATTGATGTAA AAAAAGAGAA |
| 351 | CGCTAAAATT ATTGTAACTG GATGCATGAC TTCCAACCAC AAAGATGAGC |
| 401 | TTAAACCCTG GATGTCACAC ATCCATTACC TACTAGGTTC TGGGGATGTT |
| 451 | GAGAATATTC TTTCTGCTAT TGAGTCTCGT GAATCTGGAG AAAAAATCTC |
| 501 | TGCAAAGAGT TACATTGAGA TGGGAGAAGT TCCAAGACAG CTTTCCACAC |
| 551 | CAAAACACTA TGCCTATTTA AAAGTTGCTG AGGGCTGTAG AAAACGTTGT |
| 601 | GCTTTTTGTA TTATTCCTTC CATTAAAGGA AAGCTCCGCA GCAAACCTCT |
| 651 | GGATCAAATT CTTAAAGAAT TCCGCATCCT TGTAAACAAG AGTGTGAAAG |
| 701 | AGATTATATT GATAGCTCAA GACCTAGGAG ATTATGGAAA GGATCTCTCT |
| 751 | ACAGACCGCA GTTCGCAGCT AGAATCACTA TTACATGAGT TACTGAAAGA |
| 801 | GCCTGGTGAT TATTGGCTGC GGATGTTGTA TTTATATCCT GATGAAGTGA |
| 851 | GTGATGGCAT TATAGATCTT ATGCAATCTA ATCCCAAACT TCTTCCCTAT |
| 901 | GTAGATATTC CCTTACAGCA CATTAACGAC CGTATTTTAA AGCAAATGCG |
| 951 | AAGAACGACT TCTAGGGAGC AAATCCTAGG ATTCCTAGAA AAATTACGTG |
| 1001 | CCAAGGTTCC TCAGGTCTAT ATCCGTTCTT CTGTTATTGT GGGTTTCCCC |
| 1051 | GGTGAAACTC AGGAAGAATT CCAGGAGTTA GCTGATTTTA TTGGTGAGGG |
| 1101 | TTGGATTGAT AATCTCGGAA TTTTCTTGTA CTCTCAAGAA GCGAATACCC |
| 1151 | CGGCAGCAGA ACTCCCTGAC CAGATACCAG AAAAAGTTAA AGAATCGAGG |
| 1201 | TTGAAAATTC TATCTCAAAT TCAGAAACGC AATGTGGATA AACATAATCA |
| 1251 | GAAGCTCATT GGGGAAAAAA TAGAAGCAGT TATTGATAAC TATCATCCTG |
| 1301 | AAACGAATCT TTTACTCACT GCAAGGTTCT ATGGACAAGC TCCTGAAGTG |
| 1351 | GACCCTTGTA TTATTGTAAA TGAGGCGAAG CTTGTTTCTC ATTTTGGAGA |
| 1401 | AAGATGCTTT ATAGAAATCA CAGGGACTGC TGGTTACGAC CTTGTAGGGC |
| 1451 | GTGTTGTAAA AAAATCTCAG AACCAAGCTT TGCTAAAAAC TAGCAAAGCT |
| 1501 | TAG |

The PSORT algorithm predicts cytoplasm (0.1296).

The protein was expressed in E. coli and purified as a GST-fusion product (FIG. 118A) and also as a his-tagged product. The recombinant proteins were used to immunize mice, whose sera were used in a Western blot (FIG. 118B) and for FACS analysis.

These experiments show that cp6526 is a surface-exposed and immunoaccessible protein, and that it is a useful immunogen. These properties are not evident from the sequence alone.

### Example 119

The following C. pneumoniae protein (PID 4376528) was expressed <SEQ ID 237; cp6528>:

| | |
|---|---|
| 1 | MKNNINNNEC YFKLDSTVDG DLLAANLKTF DTQAQGISST ETFSVQGNAT |
| 51 | FKDQVSATGL TSGTTYNLNA QNFTSSQISI DFKNNRLSNC ALPKEDCDPV |
| 101 | PANYVRSPEY FFCSKPLIGD FDFNSGESYL PLTGSEYTLY QSRNVNSIFR |
| 151 | FIGWKQSTRE LTVGGNTAIQ FLAAGTYIVS FTVGKRWGWN NGWGGAIYIN |
| 201 | NGLGQVQCES TIYSGGGYAT IGTLGTSIYR ASVDVAPNPN DPNASDRYRA |
| 251 | GIFYLSNGGS SAGIGNYSFS LLYYPDDRG* |

The cp6528 nucleotide sequence <SEQ ID 238> is:

| | |
|---|---|
| 1 | ATGAAAAACA ATATTAATAA TAATGAGTGC TATTTTAAAT TAGACTCAAC |
| 51 | TGTAGATGGT GATTTGTTAG CAGCCAATCT CAAGACCTTT GATACACAGG |
| 101 | CCCAAGGAAT CTCATCGACT GAAACATTTT CTGTTCAGGG GAATGCAACA |
| 151 | TTTAAAGATC AAGTTTCAGC AACTGGATTA ACTTCAGGAA CTACTTATAA |
| 201 | TTTAAATGCA CAAAACTTTA CTTCCTCCCA AATCTCTATA GATTTTAAAA |
| 251 | ATAATCGTCT GAGTAATTGT GCATTGCCAA AAGAAGACTG CGATCCGGTG |
| 301 | CCAGCGAATT ATGTTCGTTC TCCCGAATAT TTTTTCTGTT CCAAGCCTCT |
| 351 | GATCGGAGAT TTTGATTTTA ACTCAGGGGA ATCTTATTTG CCTCTGACTG |
| 401 | GTTCGGAATA TACTCTATAT CAGTCACGTA ATGTAAATAG TATATTTCGT |
| 451 | TTTATAGGAT GGAAGCAAAG TACACGAGAA TTAACTGTAG GGGGAAATAC |
| 501 | TGCGATACAA TTTCTTGCAG CAGGAACCTA TATCGTTTCA TTTACTGTTG |
| 551 | GTAAACGGTG GGGATGGAAT AATGGTTGGG GAGGAGCCAT TTATATCAAT |
| 601 | AATGGTTTAG GACAAGTCCA ATGTGAAAGC ACGATTTATA GTGGTGGAGG |
| 651 | GTATGCAACA ATAGGTACAC TGGGGACCTC AATATATAGA GCCTCTGTAG |
| 701 | ATGTAGCTCC TAATCCTAAT GATCCGAATG CTTCGGATCG CTATAGAGCG |
| 751 | GGTATTTTCT ATCTCAGTAA CGGTGGTTCT AGTGCAGGTA TAGGGAATTA |
| 801 | CTCCTTTTCT CTTCTCTATT ATCCGGACGA TAGAGGGTAG |

The PSORT algorithm predicts cytoplasm (0.1668).

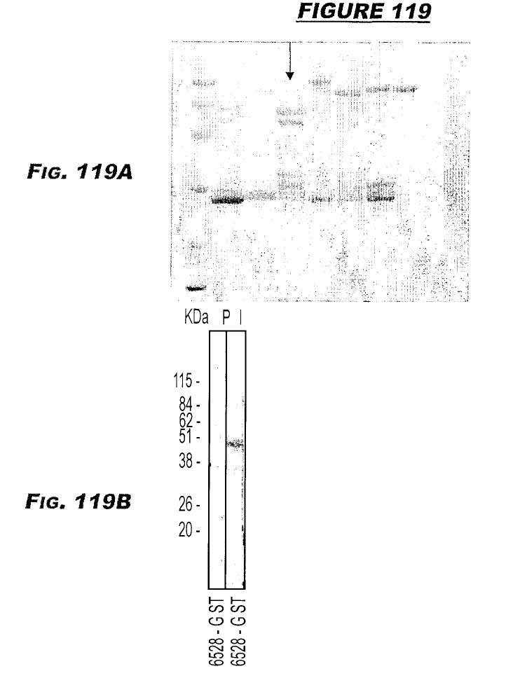

The protein was expressed in E. coli and purified as a GST-fusion product (FIG. 119A). The recombinant protein was used to immunize mice, whose sera were used in a Western blot (FIG. 119B) and for FACS analysis.

These experiments show that cp6528 is a surface-exposed and immunoaccessible protein, and that it is a useful immunogen. These properties are not evident from the sequence alone.

### Example 120

The following C. pneumoniae protein (PID 4376627) was expressed <SEQ ID 239; cp6627>:

| | |
|---|---|
| 1 | MKCSPLTLVP HIFLKNDCEC HRSCSLKIRT IARLILGLVL ALVSALSFVF |
| 51 | LAAPISYAIG GTLALAAIVI LIITLVVALL AKSKVLPIPN ELQKIIYNRY |
| 101 | PKEVFYFVKT HSLTVNELKI FINCWKSGTD LPPNLHKKAE AFGIDILKSI |
| 151 | DLTLFPEFEE ILLQNCPLYW LSHFIDKTES VAGEIGLNKT QKVYGLLGPL |
| 201 | AFHKGYTTIF HSYTRPLLTL ISESQYKFLY SKASKNQWDS PSVKKTCEEI |
| 251 | FKELPHNMIF RKDVQGISQF LFLFFSHGIT WEQAQMIQLI NPDNWKMLCQ |
| 301 | FDKAGGHCSM ATFGGFLNTE TNMFDPVSSN YEPTVNFMTW KELKVLLEKV |
| 351 | KESPMHPASA LVQKICVNTT HHQNLLKRWQ FVRNTSSQWT SSLPQYAFHA |
| 401 | QTYKLEKKIE SSLPIRSSL* |

The cp6627 nucleotide sequence <SEQ ID 240> is:

| | |
|---|---|
| 1 | ATGAAGTGTA GTCCTTTAAC ACTAGTTCCC CATATATTTT TAAAAAATGA |
| 51 | CTGCGAATGT CATAGATCTT GTTCTTTAAA AATTAGGACA ATTGCCCGAC |
| 101 | TCATTCTTGG GCTTGTTCTA GCTCTTGTTA GCGCACTTTC TTTTGTTTTC |
| 151 | CTTGCTGCGC CGATTAGCTA TGCTATTGGA GGAACTTTAG CTTTAGCCGC |
| 201 | TATCGTAATC TTGATTATAA CGCTAGTCGT AGCACTGCTA GCTAAATCAA |
| 251 | AGGTTCTGCC CATCCCCAAC GAACTTCAGA AGATTATTTA CAATCGCTAT |
| 301 | CCTAAAGAAG TCTTTTATTT CGTGAAAACA CACTCCCTGA CTGTTAACGA |
| 351 | ATTAAAAATA TTTATTAATT GCTGGAAAAG CGGTACAGAC CTGCCTCCGA |
| 401 | ATTTACATAA AAAAGCAGAG GCTTTCGGGA TCGATATTCT AAAATCTATA |
| 451 | GATTTAACCC TGTTTCCAGA GTTCGAAGAG ATTCTTCTTC AAAACTGCCC |
| 501 | GTTATACTGG CTCTCCCATT TTATAGACAA AACTGAATCT GTTGCTGGGG |
| 551 | AAATCGGATT AAATAAAACA CAAAAAGTTT ATGGTTTACT TGGGCCCTTA |
| 601 | GCGTTTCATA AAGGATATAC AACTATTTTC CACTCTTATA CACGCCCTCT |
| 651 | ACTAACATTA ATCTCAGAAT CACAGTATAA GTTCCTATAT AGTAAAGCGT |
| 701 | CTAAGAATCA ATGGGATTCT CCTTCTGTGA AAAAAACCTG CGAAGAAATA |
| 751 | TTCAAGGAAC TCCCCCACAA TATGATTTTC CGGAAGGATG TTCAAGGAAT |
| 801 | CTCACAATTC TTATTTCTTT TCTTTTCTCA TGGTATCACT TGGGAACAGG |
| 851 | CTCAGATGAT TCAACTTATA AATCCTGATA ATTGGAAAAT GTTGTGTCAG |
| 901 | TTTGATAAAG CAGGAGGCCA CTGTTCCATG GCAACATTTG GAGGCTTTTT |
| 951 | GAATACTGAA ACAAATATGT TCGATCCAGT ATCCTCTAAC TATGAACCTA |
| 1001 | CAGTGAACTT CATGACGTGG AAAGAATTGA AGGTTTTACT AGAGAAAGTA |
| 1051 | AAAGAAAGTC CTATGCACCC AGCGAGTGCT CTTGTTCAGA AGATATGCGT |
| 1101 | AAATACAACG CACCATCAAA ATCTGTTAAA ACGATGGCAA TTTGTTCGTA |
| 1151 | ATACGAGTTC ACAATGGACA TCAAGCTTAC CTCAGTATGC TTTCCACGCC |
| 1201 | CAAACCTACA AACTAGAGAA AAAAATAGAA AGCAGTCTCC CTATACGATC |
| 1251 | TTCCCTATAA |

The PSORT algorithm predicts inner membrane (0.7198).

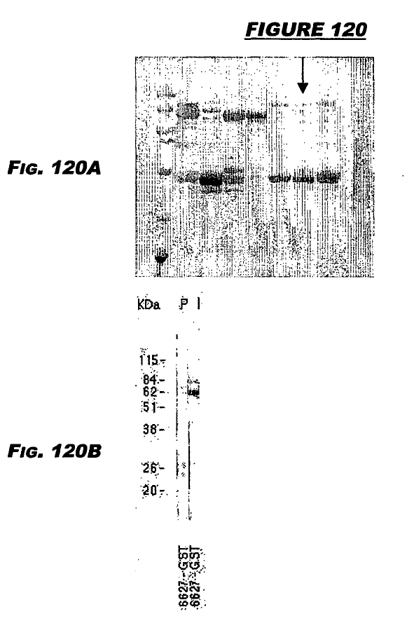

The protein was expressed in E. coli and purified as a GST-fusion product (FIG. 120A). The recombinant protein was used to immunize mice, whose sera were used in a Western blot (FIG. 120B) and for FACS analysis.

These experiments show that cp6627 is a surface-exposed and immunoaccessible protein, and that it is a useful immunogen. These properties are not evident from the sequence alone.

### Example 121

The following C. pneumoniae protein (PID 4376629) was expressed <SEQ ID 241; cp6629>:

| | |
|---|---|
| 1 | MSNITSPVIQ NNRSCNYYFE LKNSTTIHIV ISAILLCGAL IAFLCVAAPV |
| 51 | SYILSGALLG LGLLIALIGV ILGIKKITPM ISSKEQVFPQ ELVNRIRAHY |
| 101 | PKFVSDFVSE AKPNLKDLIS FIDLLNQLHS EVGSSTNYNV SEELQQKIDT |
| 151 | FEGIARLKNE VRTASLKRLE SAASSRPLFP SLPKILQKVF PFFWLGEFIS |
| 201 | AGSKVVELHR VKKIGGSLEE DLSDYIKPEM LPTYWLIPLD FRPTNSSILN |
| 251 | LHTLVLARVL TRDVFQHLKY AALNGEWNLN HSDLNTMKQQ LFAKYHAAYQ |
| 301 | SYKHLSQPSL QEDEFYNLLL CIFKHRYSWK QMSLIKTVPA DLWENLCCLT |
| 351 | LDHTGRPQDM EFASLIGTLY TQGLIHKESE AFLSSLTLLS LDQFKTIRRQ |
| 401 | STNIAMFLEN LATHNSTFRS LPPITVHPLK RSVFSQPEED ESSLLIG* |

The cp6629 nucleotide sequence <SEQ ID 242> is:

| | |
|---|---|
| 1 | ATGAGTAATA TAACCTCGCC AGTTATTCAA AATAATCGCT CTTGTAATTA |
| 51 | TTATTTTGAA TTAAAGAATT CAACCACTAT TCATATTGTT ATCAGTGCCA |
| 101 | TCTTACTCTG CGGAGCTTTG ATAGCTTTCT TGTGTGTAGC AGCTCCTGTT |
| 151 | TCCTATATTC TAAGTGGCGC ATTGTTAGGA TTAGGATTAT TAATAGCCTT |
| 201 | GATTGGTGTG ATTTTAGGAA TAAAAAAAAT CACGCCTATG ATTTCATCAA |
| 251 | AAGAACAAGT ATTCCCCCAA GAACTCGTAA ATAGAATCAG GGCGCACTAT |
| 301 | CCTAAATTTG TCTCTGATTT TGTTTCAGAA GCTAAACCAA ATCTTAAAGA |
| 351 | TCTCATAAGT TTTATTGATC TTCTAAATCA ATTGCACTCT GAAGTTGGAT |
| 401 | CATCTACAAA TTACAACGTA TCTGAAGAAC TACAACAGAA AATAGATACG |
| 451 | TTCGAGGGTA TCGCACGCTT AAAAAATGAA GTCCGTACTG CTTCTCTTAA |
| 501 | AAGACTTGAA AGCGCTGCTT CTTCCCGTCC CCTCTTCCCC TCTTTACCAA |
| 551 | AAATCTTACA AAAGGTATTT CCATTTTTCT GGTTAGGAGA GTTTATTTCT |
| 601 | GCAGGCAGCA AGGTTGTAGA GCTCCATCGA GTTAAGAAAA TTGGAGGCAG |
| 651 | CCTCGAAGAA GACCTTAGTG ATTATATAAA ACCAGAGATG CTTCCTACCT |
| 701 | ATTGGTTGAT TCCTTTAGAT TTTAGACCAA CAAATTCCTC TATTCTAAAT |
| 751 | CTACACACAT TAGTTTTAGC TAGAGTCTTA ACTCGTGATG TTTTTCAACA |
| 801 | TCTTAAGTAT GCAGCATTAA ATGGCGAGTG GAACCTGAAT CATAGTGATC |
| 851 | TAAATACTAT GAAACAGCAG CTCTTTGCTA AATATCATGC GGCGTATCAA |
| 901 | TCCTATAAAC ATCTATCTCA ACCCTCTCTT CAAGAGGATG AATTCTATAA |
| 951 | CCTGCTCTTG TGTATTTTTA AGCATAGGTA CTCGTGGAAG CAGATGTCCT |
| 1001 | TAATAAAAAC AGTCCCGGCT GATTTATGGG AAAACCTCTG TTGCTTGACT |
| 1051 | TTAGACCATA CAGGACGACC CCAAGACATG GAATTTGCCT CTCTAATTGG |
| 1101 | TACTCTCTAC ACACAAGGCC TAATTCATAA AGAAAGCGAA GCATTTCTTT |
| 1151 | CTTCATTGAC ACTCCTTAGT TTAGATCAGT TTAAAACGAT CCGTCGTCAG |
| 1201 | TCAACCAATA TAGCGATGTT CCTTGAGAAT TTAGCAACTC ATAATTCCAC |
| 1251 | CTTTAGAAGC TTACCACCTA TAACAGTCCA TCCACTCAAG AGAAGCGTCT |
| 1301 | TCTCCCAACC TGAAGAAGAC GAGTCCTCCC TGCTGATAGG TTAG |

The PSORT algorithm predicts inner membrane (0.5776).

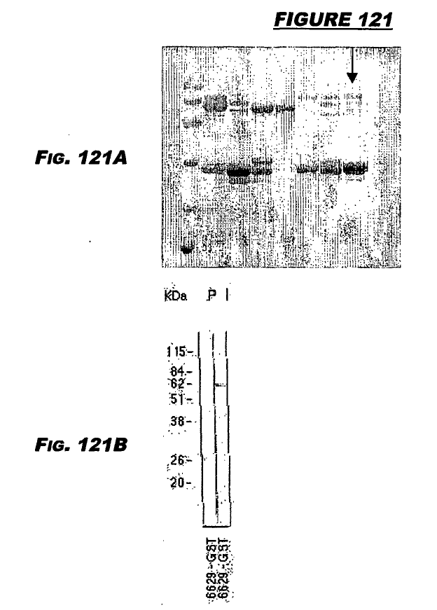

The protein was expressed in E. coli and purified as a GST-fusion product (FIG. 121A). The recombinant protein was used to immunize mice, whose sera were used in a Western blot (FIG. 121B) and for FACS analysis.

These experiments show that cp6629 is a surface-exposed and immunoaccessible protein, and that it is a useful immunogen. These properties are not evident from the sequence alone.

### Example 122

The following C. pneumoniae protein (PID 4376732) was expressed <SEQ ID 243; cp6732>:

| | |
|---|---|
| 1 | MEMMSPFQQP EQCHFDVVGS FLRPESLTRA RSDFEEGRIV YEQMRVVEDA |
| 51 | AIRNLIKKQT EAGLIFFTDG EFRRYSWDFD FMWGFHGVDR RRDSNDPEIG |
| 101 | VYLKDKISVS KHPFIEHFEF VKTFEKGNAK AKQTIPSPSQ FFHEMIFAPN |
| 151 | LKNTRKFYPT NQELIDDIVF YYRQVIQDLY AAGCRNLQLD DCAWCRLLDI |
| 201 | RAPSWYGVDS HDRLQEILEQ FLWIHNLVMK DRPEDLFVSL HVCRGDYQAE |
| 251 | FFSRRAYDSI EEPLFAKTDV DSYHYYWALD DKYSGGAEPL AYVSGEKHVC |
| 301 | LGLISSNHSC IEDRDAVVSR IYEAASYIPL ERLSLSPQCG FASCEGDHRM |
| 351 | TEEEQWKKIA FVKEIAKEIW G* |

The cp6732 nucleotide sequence <SEQ ID 244> is:

| | |
|---|---|
| 1 | ATGGAAATGA TGAGCCCATT CCAACAACCT GAGCAATGTC ATTTTGATGT |
| 51 | TGTGGGAAGT TTCTTACGTC CTGAAAGTCT TACACGAGCA CGCTCTGATT |
| 101 | TTGAAGAAGG AAGAATTGTC TATGAGCAGA TGCGAGTTGT CGAAGATGCT |
| 151 | GCTATTCGTA ATCTCATAAA AAAGCAAACA GAAGCAGGTC TTATCTTTTT |
| 201 | TACTGATGGG GAATTCCGTA GGTATAGTTG GGATTTCGAC TTTATGTGGG |
| 251 | GATTCCATGG CGTGGATCGT CGCAGGGACT CTAATGACCC TGAAATTGGA |
| 301 | GTGTATCTTA AAGATAAAAT CTCCGTATCA AAACATCCGT TTATAGAACA |
| 351 | TTTCGAGTTT GTCAAAACTT TTGAGAAGGG AAATGCAAAA GCAAAACAAA |
| 401 | CGATTCCTTC TCCATCACAA TTTTTCCATG AGATGATTTT TGCTCCTAAT |
| 451 | CTGAAAAATA CTCGGAAGTT TTATCCTACG AATCAAGAGC TAATTGATGA |
| 501 | TATTGTCTTT TATTATCGCC AAGTCATCCA AGATCTTTAT GCTGCAGGTT |
| 551 | GTCGTAATTT GCAGTTGGAC GATTGTGCTT GGTGTCGCCT CTTGGATATA |
| 601 | CGAGCGCCTT CTTGGTATGG TGTTGATTCT CATGACAGGT TGCAGGAAAT |
| 651 | TTTAGAACAG TTTTTATGGA TCCATAATTT AGTGATGAAG GATAGACCCG |
| 701 | AGGATCTTTT TGTAAGTCTG CATGTCTGTC GTGGTGATTA TCAGGCCGAG |
| 751 | TTTTTCTCTA GACGAGCTTA TGATTCTATA GAGGAGCCTT TATTTGCTAA |
| 801 | GACCGATGTG GATAGTTATC ACTATTATTG GGCTCTTGAT GATAAGTATT |
| 851 | CAGGAGGTGC TGAGCCTTTA GCTTACGTCT CTGGAGAGAA ACACGTCTGC |
| 901 | TTGGGATTGA TCTCCAGCAA CCATTCTTGT ATTGAAGATC GAGATGCTGT |
| 951 | GGTTTCTCGT ATTTATGAAG CTGCGAGCTA CATTCCCTTA GAGAGACTTT |
| 1001 | CTTTGAGCCC GCAATGTGGG TTTGCTTCTT GTGAGGGAGA CCATAGAATG |
| 1051 | ACTGAAGAAG AACAGTGGAA GAAGATCGCC TTTGTGAAAG AGATTGCTAA |
| 1101 | AGAGATCTGG GGATAA |

The PSORT algorithm predicts cytoplasm (0.2196).

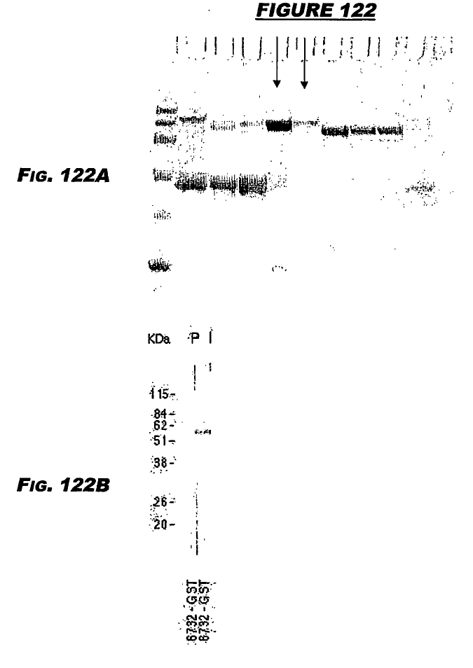

The protein was expressed in E. coli and purified as a GST-fusion product (FIG. 122A). The recombinant protein was used to immunize mice, whose sera were used in a Western blot (FIG. 122B) and for FACS analysis.

These experiments show that cp6732 is a surface-exposed and immunoaccessible protein, and that it is a useful immunogen. These properties are not evident from the sequence alone.

### Example 123

The following C. pneumoniae protein (PID 4376738) was expressed <SEQ ID 245; cp6738>:

| | |
|---|---|
| 1 | VWLRFLLLVS YDEKEKDVVV VCNHSEPNIL GLPPEAVSQL IEELSDEGYS |
| 51 | YLNVVRCDLS GETTVQQRLL LNADEGRSMT VVISELPEGH PDIRNLQLAS |
| 101 | ERIFVSREKE AADAYASGCK VVAFDDEHLP WVSSHIAYAE EIREKQEQTM |
| 151 | QGSLTEEQLG ALLCNTVSTE KNLAFALDAV IKQSVWRFRN PDLFAYEREA |
| 201 | LEASVTDALV SYVSNLDMIP YTSSQGIVIE DSSIVRTSQE HTLIVNCAAF |
| 251 | DKLASQIEFL CPSDVLPISG KDPLISDDED EELNPKVSSA ADSKDKT* |

The cp6738 nucleotide sequence <SEQ ID 246> is:

| | |
|---|---|
| 1 | GTGTGGCTGC GCTTTTTACT TTTAGTGTCC TATGATGAGA AGGAGAAAGA |
| 51 | CGTAGTTGTC GTTTGTAATC ATTCTGAACC TAATATCCTC GGCCTGCCTC |
| 101 | CTGAAGCAGT CTCTCAGCTT ATTGAAGAGC TTAGCGATGA AGGCTATAGC |
| 151 | TATCTGAATG TAGTGCGTTG TGATCTCTCC GGGGAGACTA CGGTTCAACA |
| 201 | ACGTCTGCTA TTGAATGCCG ATGAAGGGAG ATCTATGACG GTGGTGATCT |
| 251 | CAGAGCTTCC TGAAGGGCAC CCCGATATTC GGAATTTGCA GTTGGCATCC |
| 301 | GAAAGAATTT TTGTTTCTCG TGAAAAAGAA GCTGCTGATG CCTATGCTTC |
| 351 | AGGATGTAAA GTGGTCGCTT TCGATGATGA GCATCTCCCT TGGGTCTCCA |
| 401 | GTCATATTGC CTACGCGGAG GAGATCAGAG AGAAACAAGA ACAAACAATG |
| 451 | CAAGGGTCTT TAACTGAAGA GCAGTTAGGA GCACTCCTCT GCAACACAGT |
| 501 | CTCCACAGAG AAAAATCTAG CCTTTGCTCT AGACGCCGTG ATAAAACAGT |
| 551 | CTGTGTGGAG ATTCCGCAAT CCGGATCTTT TTGCTTATGA GAGAGAAGCT |
| 601 | CTAGAGGCTT CAGTAACAGA TGCTTTAGTA TCTTACGTTT CAAATTTAGA |
| 651 | CATGATACCG TACACAAGTT CTCAGGGCAT AGTCATAGAA GATAGTAGTA |
| 701 | TCGTCCGTAC CTCTCAAGAG CATACACTCA TTGTGAACTG TGCAGCATTC |
| 751 | GATAAGTTAG CGAGCCAAAT AGAGTTCTTA TGCCCCAGTG ACGTGTTGCC |
| 801 | CATTTCTGGT AAAGACCCTT TGATTTCTGA TGATGAGGAT GAGGAACTGA |
| 851 | ATCCTAAAGT TTCATCTGCT GCAGACTCTA AAGATAAAAC CTAG |

The PSORT algorithm predicts cytoplasm (0.1587).

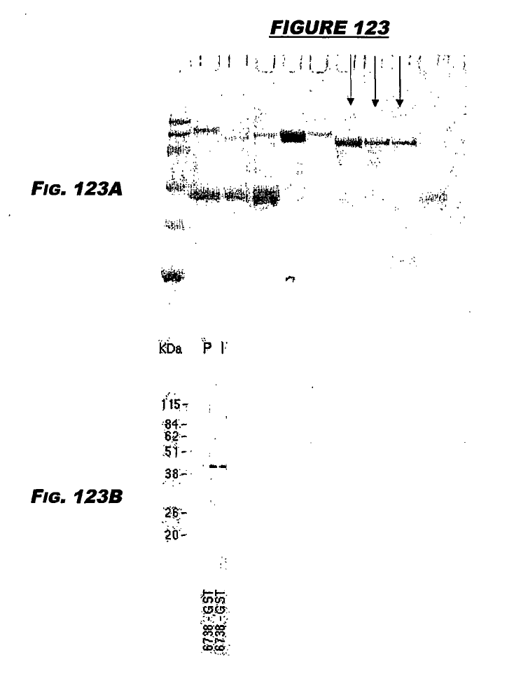

The protein was expressed in E. coli and purified as a GST-fusion product (FIG. 123A). The recombinant protein was used to immunize mice, whose sera were used in a Western blot (FIG. 123B) and for FACS analysis.

These experiments show that cp6738 is a surface-exposed and immunoaccessible protein, and that it is a useful immunogen. These properties are not evident from the sequence alone.

### Example 124

The following C. pneumoniae protein (PID 4376739) was expressed <SEQ ID 247; cp6739>:

| | |
|---|---|
| 1 | MTHCLHGWFS VVRHHFVQAF NFSRPLYSRI THFALGVIKA IPIVGHLVMG |
| 51 | VDWLISHCFE RGVSHPGFPS DIAPILKVEK IAGRDHISRI ENQLKSLRKT |
| 101 | IEVEDLDKVH GQYQENPYAD MASSEVLKLD KGVHVSELGK AFSRVRNRIT |
| 151 | RSYSYAPTPQ LDSIAIVGID LVSPEEQENL VRLANEVIQL YPKSKTTLYL |
| 201 | LIDFNKEWVG DISSDKEKQL RSLGLHSEVQ CLSVLEPQGA EGEDTKHFDL |
| 251 | MVGCYGKDSY LREGKILQQA LGTSLGTVPW VNVMHTLPSR YRSRLSLPIN |
| 301 | TEKDKTELYK EISRTHHQLH TLGMGLGAQD SGLLLDRQRL HAPLSQGSHC |
| 351 | HSYLADLTHE ELKILLFSAF VDAKNISKKE LREVSLNFAN DTSVECGCAF |
| 401 | YF* |

The cp6739 nucleotide sequence <SEQ ID 248> is:

| | |
|---|---|
| 1 | ATGACTCATT GCTTACATGG TTGGTTTTCT GTAGTTCGTC ATCACTTTGT |
| 51 | GCAGGCGTTT AATTTCTCAC GTCCTTTATA TTCTCGAATT ACCCACTTCG |
| 101 | CTTTAGGGGT GATTAAGGCC ATCCCCATTG TAGGGCATCT TGTTATGGGA |
| 151 | GTCGATTGGT TGATCTCTCA TTGCTTCGAG AGGGGAGTCT CACACCCTGG |
| 201 | GTTCCCTTCA GATATTGCTC CTATACTGAA AGTAGAAAAG ATCGCGGGCC |
| 251 | GAGATCATAT TTCTAGAATC GAAAATCAGC TAAAGAGCCT TAGGAAAACT |
| 301 | ATCGAGGTTG AAGATCTAGA TAAAGTCCAC GGGCAATATC AAGAGAATCC |
| 351 | TTATGCAGAT ATGGCCTCTA GTGAGGTTCT TAAACTCGAT AAGGGAGTTC |
| 401 | ATGTTAGCGA GCTTGGCAAA GCCTTTTCTA GAGTTCGCAA TCGCATCACC |
| 451 | AGATCCTATA GTTATGCCCC TACTCCTCAG TTGGACTCTA TAGCTATTGT |
| 501 | TGGTATAGAT CTCGTCAGTC CTGAAGAACA AGAGAATTTA GTACGCTTGG |
| 551 | CGAATGAGGT CATTCAACTC TATCCCAAAT CAAAGACAAC TCTATATCTT |
| 601 | CTTATCGATT TTAATAAGGA GTGGGTAGGG GATATCTCCT CTGATAAGGA |
| 651 | AAAACAGCTC CGTTCTCTAG GTCTACATTC TGAAGTTCAG TGTCTTTCCG |
| 701 | TCTTGGAACC TCAGGGTGCC GAGGGCGAAG ATACGAAACA CTTTGACCTT |
| 751 | ATGGTCGGCT GTTATGGGAA GGATTCTTAC TTAAGGGAGG GTAAAATTTT |
| 801 | ACAGCAGGCC CTAGGGACTT CGTTAGGTAC TGTTCCCTGG GTGAATGTTA |
| 851 | TGCACACATT GCCATCTAGG TATAGATCTC GGCTTTCCTT ACCTATAAAT |
| 901 | ACCGAAAAGG ATAAGACAGA GCTTTATAAA GAGATTTCTC GTACACACCA |
| 951 | TCAGTTGCAT ACTTTGGGAA TGGGACTTGG AGCCCAGGAT TCAGGATTGC |
| 1001 | TCTTAGACCG GCAACGACTC CATGCTCCTT TATCTCAAGG GTCTCACTGC |
| 1051 | CATTCCTATC TTGCAGATCT CACCCATGAA GAGCTGAAAA TTTTGTTATT |
| 1101 | TTCAGCATTT GTGGATGCTA AGAACATAAG TAAGAAAGAG CTTCGTGAGG |
| 1151 | TATCTCTAAA TTTTGCTAAC GATACTTCCG TAGAGTGTGG CTGCGCTTTT |
| 1201 | TACTTTTAG |

The PSORT algorithm predicts inner membrane (0.2190).

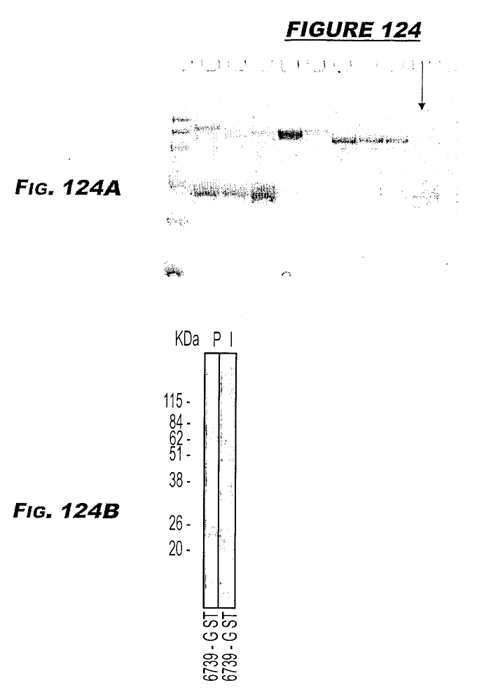

The protein was expressed in E. coli and purified as a GST-fusion product (FIG. 124A). The recombinant protein was used to immunize mice, whose sera were used in a Western blot (FIG. 124B) and for FACS analysis.

These experiments show that cp6739 is a surface-exposed and immunoaccessible protein, and that it is a useful immunogen. These properties are not evident from the sequence alone.

### Example 125

The following C. pneumoniae protein (PID 4376741) was expressed <SEQ ID 249; cp6741>:

| | |
|---|---|
| 1 | MASCLSAWFS IVREHFYRAF DFSLPFCARI TEFVLGVIKG IPVVGHIIVG |
| 51 | IEWLVSRYLE SFVTKPTFVS DVVSLLKTEK VAGRDHIARV VETLKRQRVA |
| 101 | VAPEDEDKVH GKIPVHPFGG IQPVEVLTLY PEVQDATLGL AFSKIRNRVR |
| 151 | QAYLQAPRPK LQKIYIIGND MNPFEVDDFL HLARLCNETQ RLYPDATISL |
| 201 | YLTASGGRNA MDKKNRKLLS DCELNPKIAC LDFNQGDVVK QATCDCWMVY |
| 251 | HGENDQGTLN QIQEELEKSG EETPWIHVGQ KPLSQSLWDF SPFSSLEMKG |
| 301 | DKEKALEYSE LEKEQLYSRL VYVGERSSVL SLGFGDSRSG ILMDPKRVHA |
| 351 | PLSEGHYCHS YLADLENPGL QKTILAAFLN PKELSSTILQ PISLNLILNS |
| 401 | KTYLRQHFGF FERMSRSDRN VVVVVCDSWW GTDWKEEPSF QHFIMELECR |
| 451 | GYSHFNIFAF RSNSMCVEER RILNESSQEK AFTMIFCEDS VSQGDIRCLH |
| 501 | LASEGMLCGK ECYAVDVYTS GCANFMMEEV LTLERESNLW NRKHGLWKRE |
| 551 | VRKQKQEAAL DQDESEIYVC NQLTAQQNFA CS* |

The cp6741 nucleotide sequence <SEQ ID 250> is:

| | |
|---|---|
| 1 | ATGGCTTCTT GTTTATCTGC CTGGTTTTCT ATAGTTCGTG AGCACTTTTA |
| 51 | TCGAGCCTTT GATTTTTCTT TGCCGTTTTG TGCTCGTATT ACGGAATTTG |
| 101 | TATTAGGGGT CATCAAGGGG ATCCCTGTTG TGGGTCACAT TATTGTTGGG |
| 151 | ATAGAGTGGC TCGTTTCTAG GTATTTAGAG AGTTTCGTGA CCAAGCCGAC |
| 201 | ATTTGTCTCT GATGTGGTGA GTCTTCTGAA AACAGAGAAA GTTGCTGGTC |
| 251 | GCGATCACAT TGCTCGTGTA GTGGAGACTT TGAAGAGGCA GAGAGTCGCT |
| 301 | GTGGCTCCTG AAGATGAGGA TAAGGTCCAT GGGAAGATTC CTGTGCATCC |
| 351 | TTTCGGGGGA ATCCAACCTG TAGAAGTTCT CACTCTCTAT CCCGAAGTTC |
| 401 | AAGATGCAAC GTTAGGGCTT GCCTTCTCTA AAATTCGTAA TCGTGTAAGA |
| 451 | CAGGCGTATT TGCAAGCTCC ACGGCCAAAA CTGCAGAAGA TTTACATCAT |
| 501 | AGGAAACGAT ATGAATCCTT TTGAAGTTGA CGACTTCTTG CATCTAGCCC |
| 551 | GTCTCTGTAA TGAAACTCAA AGACTCTATC CTGACGCTAC GATTTCTCTA |
| 601 | TATCTAACAG CTTCTGGTGG TCGCAATGCT ATGGACAAAA AGAATCGGAA |
| 651 | GTTACTTAGT GATTGCGAAC TAAACCCCAA GATTGCTTGT TTGGACTTTA |
| 701 | ATCAGGGTGA TGTAGTCAAA CAAGCAACTT GTGACTGTTG GATGGTGTAT |
| 751 | CATGGGGAGA ATGATCAAGG TACGTTGAAT CAGATTCAGG AAGAGTTAGA |
| 801 | AAAGTCAGGG GAGGAAACCC CTTGGATTCA TGTGGGGCAA AAGCCTCTTT |
| 851 | CACAATCCTT GTGGGATTTC TCTCCATTTT CATCTTTGGA GATGAAGGGA |
| 901 | GATAAAGAGA AAGCTCTAGA GTACTCTGAA TTAGAAAAAG AACAGCTATA |
| 951 | TTCTCGATTG GTATACGTAG GAGAGCGCTC TTCGGTTCTT AGTTTGGGGT |
| 1001 | TTGGAGATAG TCGGTCAGGG ATCTTGATGG ACCCAAAACG GGTGCATGCT |
| 1051 | CCCTTATCTG AAGGGCATTA TTGTCATTCC TACCTTGCAG ACTTAGAAAA |
| 1101 | TCCCGGGTTA CAAAAAACAA TTTTAGCGGC ATTTCTGAAT CCTAAGGAGT |
| 1151 | TGAGCAGTAC CATACTGCAA CCTATATCTC TAAATCTTAT CTTAAATAGC |
| 1201 | AAAACTTACT TAAGGCAGCA CTTTGGCTTT TTTGAGAGGA TGAGCAGAAG |
| 1251 | TGATCGCAAT GTGGTTGTCG TTGTATGTGA TTCTTGGTGG GGTACCGACT |
| 1301 | GGAAGGAGGA GCCAAGCTTC CAACACTTTA TTATGGAGCT AGAGTGTCGA |
| 1351 | GGGTATTCGC ACTTCAATAT TTTTGCCTTT AGATCTAATA GCATGTGTGT |
| 1401 | AGAAGAACGT AGGATCTTAA ATGAAAGTTC TCAAGAGAAA GCCTTTACCA |
| 1451 | TGATTTTCTG TGAGGATTCA GTATCTCAAG GAGATATCCG CTGTTTGCAT |
| 1501 | TTGGCGTCTG AAGGAATGCT TTGTGGTAAA GAGTGCTATG CTGTCGATGT |
| 1551 | CTATACGTCA GGATGCGCGA ACTTTATGAT GGAAGAAGTC TTAACTTTGG |
| 1601 | AGCGAGAATC TAATCTGTGG AATAGAAAGC ATGGTCTTTG GAAAAGAGAA |
| 1651 | GTTAGAAAAC AGAAACAAGA AGCTGCTTTG GATCAAGACG AGAGCGAGAT |
| 1701 | TTACGTTTGT AATCAGCTGA CGGCGCAACA GAACTTCGCT TGTTCTTGA |

The PSORT algorithm predicts inner membrane (0.2869).

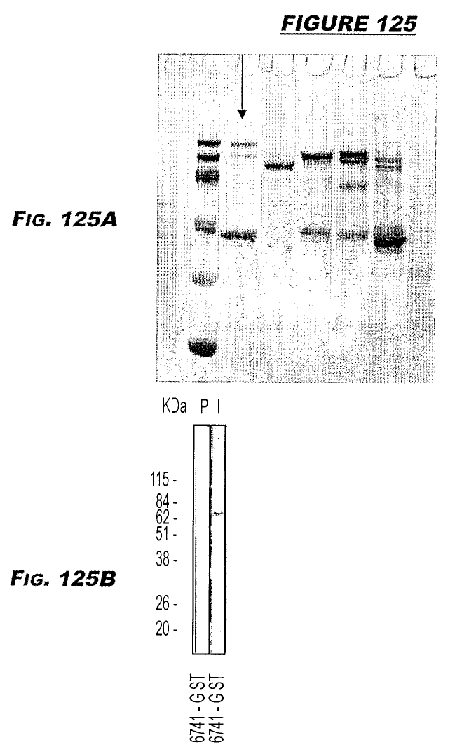

The protein was expressed in E. coli and purified as a GST-fusion product (FIG. 125A). The recombinant protein was used to immunize mice, whose sera were used in a Western blot (FIG. 125B) and for FACS analysis.

These experiments show that cp6741 is a surface-exposed and immunoaccessible protein, and that it is a useful immunogen. These properties are not evident from the sequence alone.

### Example 126

The following C. pneumoniae protein (PID 4376742) was expressed <SEQ ID 251; cp6742>:

| | |
|---|---|
| 1 | LFVSNFIFFV VMPIPYISSW ISTVRQHFVK AFDFSRPFCS RVTNFALGVI |
| 51 | KAIPIVGHIV MGMEWLVSSC VAGIITRSSF TSDVVQIVKT EKALGRDHIS |
| 101 | RVAEILQRER GTITPENQDK VHGKFPVCPF GRLKSEETLK LKPGEREGTL |
| 151 | DTVFSPIRTR VTRAYLQAPR PEIRTISIVG SKLKTPQDFS QFVSLANETQ |
| 201 | RLHPEALVCL YLTGLNRESQ MCDTTTAEKK QYLHNSGLDS RIQCKDSKED |
| 251 | DAGSPENPEL WIGYYSREQQ HNIDGQYIQQ CLGKSADPIP WIHVTEDTKD |
| 301 | FYYPPNFTSY SHTRQSTDPT SPPRLPESEG DKDSLYGQLS RSYHHEYMLG |
| 351 | LGLKPEDAGL LMDPDRIYAP LSQGHYCHSY LADIENEDLR TLVLSPFLDP |
| 401 | GNLSSEDLRP VAFNIARLPL ELDSLFFRLV AGQQEGRNIV TLAHGTPRPE |
| 451 | DLDPDSMNIL TRRLQMSGYS YLNIFSYKSR KMIVKERQFF GDRSEGKSFT |
| 501 | LILFEDPISA ADFRCLQLAA EGMVAKDLPS VADICASGCS CIQFSEMQSP |
| 551 | QAIEYRQWEA RVEDEAGEEA REPVIYSQDQ LSSMLTTQQN FVFSLDAVVK |
| 601 | QAIWRFRSKG LLTMERKALG EEFLTAIFSY LGSQERNENM GKRTTEEHEV |
| 651 | VTSFEELDRM VQVLPAEVPA DSGNDPTRPV PNPDSNPDSS QNEGS* |

The cp6742 nucleotide sequence <SEQ ID 252> is:

| | |
|---|---|
| 1 | TTGTTTGTTT CTAATTTTAT TTTTTTTGTT GTTATGCCAA TTCCCTATAT |
| 51 | TTCTTCTTGG ATTTCTACCG TTCGACAGCA TTTTGTTAAG GCGTTTGATT |
| 101 | TCTCTCGTCC CTTTTGTTCT AGGGTTACGA ATTTTGCTTT AGGGGTCATC |
| 151 | AAGGCCATCC CTATTGTAGG ACATATTGTC ATGGGGATGG AGTGGTTAGT |
| 201 | TTCTTCCTGT GTTGCCGGGA TTATTACTAG GTCCTCCTTT ACCTCAGATG |
| 251 | TCGTTCAGAT TGTAAAGACT GAGAAGGCGT TAGGTCGAGA TCATATATCT |
| 301 | CGAGTGGCGG AGATATTGCA AAGAGAAAGG GGGACCATAA CTCCTGAGAA |
| 351 | TCAAGATAAG GTGCATGGGA AGTTTCCTGT CTGTCCTTTT GGTCGTTTAA |
| 401 | AATCCGAGGA AACTTTAAAA CTTAAGCCGG GAGAAAGAGA GGGAACTTTA |
| 451 | GATACTGTAT TTTCTCCGAT TCGCACGCGC GTGACTCGTG CGTACTTACA |
| 501 | GGCCCCCCGA CCCGAAATAC GTACGATTTC TATTGTGGGT TCGAAACTTA |
| 551 | AAACTCCTCA AGATTTCTCG CAATTTGTGA GTCTCGCGAA TGAAACGCAG |
| 601 | AGACTGCATC CTGAAGCGTT AGTTTGTCTG TATTTGACAG GCTTGAATCG |
| 651 | CGAATCTCAG ATGTGCGATA CAACTACTGC AGAGAAGAAG CAGTACCTAC |
| 701 | ATAACTCAGG TCTCGACTCT AGAATCCAGT GCAAAGACAG TAAAGAAGAC |
| 751 | GACGCTGGCT CTCCTGAAAA TCCCGAACTT TGGATTGGCT ATTATTCACG |
| 801 | AGAGCAACAG CATAATATAG ACGGGCAGTA TATTCAGCAG TGTCTAGGGA |
| 851 | AGAGTGCAGA TCCAATTCCT TGGATTCATG TTACTGAAGA CACAAAGGAT |
| 901 | TTTTATTACC CACCAAACTT TACTTCATAC TCACATACAA GACAATCTAC |
| 951 | AGACCCAACA TCGCCACCAA GACTCCCTGA AAGTGAGGGG GATAAGGATT |
| 1001 | CCTTGTACGG ACAACTGAGT CGATCGTATC ACCATGAGTA TATGCTTGGT |
| 1051 | TTGGGATTAA AACCAGAGGA TGCAGGACTC CTGATGGACC CGGATAGAAT |
| 1101 | CTATGCTCCT CTATCCCAAG GGCATTATTG TCATTCCTAC CTTGCGGATA |
| 1151 | TAGAAAATGA GGATCTACGA ACTTTAGTCC TTTCGCCTTT CCTAGATCCT |
| 1201 | GGCAATCTTA GTAGCGAGGA TCTTCGTCCT GTAGCATTCA ATATCGCTAG |
| 1251 | ATTGCCATTA GAATTGGACT CGTTATTTTT CCGCCTTGTT GCGGGTCAGC |
| 1301 | AAGAAGGGAG AAACATAGTT ACCCTTGCCC ACGGAACTCC TCGTCCAGAA |
| 1351 | GATCTTGATC CTGACTCAAT GAACATTCTG ACCAGAAGAT TACAAATGTC |
| 1401 | TGGATATAGC TATTTGAACA TTTTCTCCTA TAAATCACGG AAAATGATTG |
| 1451 | TAAAAGAACG TCAGTTCTTT GGAGATCGTT CTGAAGGGAA GTCTTTCACA |
| 1501 | TTGATCTTAT TTGAGGATCC CATTAGTGCA GCAGATTTCC GTTGTTTGCA |
| 1551 | GCTAGCTGCA GAAGGTATGG TTGCTAAGGA TCTCCCCAGC GTAGCAGATA |
| 1601 | TTTGTGCCTC TGGATGTTCC TGCATTCAGT TTTCTGAGAT GCAGAGTCCT |
| 1651 | CAGGCTATTG AATATAGACA ATGGGAGGCA CGTGTCGAAG ATGAAGCAGG |
| 1701 | AGAAGAAGCC AGAGAACCAG TAATTTATTC TCAGGATCAA TTGAGCAGCA |
| 1751 | TGCTCACTAC ACAACAGAAT TTTGTATTTT CTCTAGATGC TGTGGTAAAA |
| 1801 | CAGGCGATCT GGAGATTCCG TTCGAAAGGT CTTCTTACTA TGGAAAGAAA |
| 1851 | GGCACTAGGC GAGGAGTTCT TAACTGCGAT ATTTTCCTAT TTAGGGAGTC |
| 1901 | AGGAGCGTAA TGAGAATATG GGGAAAAGAA CTACCGAAGA ACATGAGGTC |
| 1951 | GTTATCAGCT TCGAAGAGCT AGATCGCATG GTGCAAGTCC TCCCAGCCGA |
| 2001 | AGTCCCTGCA GATTCAGGCA ATGATCCTAC GCGTCCCGTT CCTAATCCAG |
| 2051 | ATAGTAACCC TGATTCCTCG CAAAATGAAG GCAGTTAG |

The PSORT algorithm predicts inner membrane (0.2338).

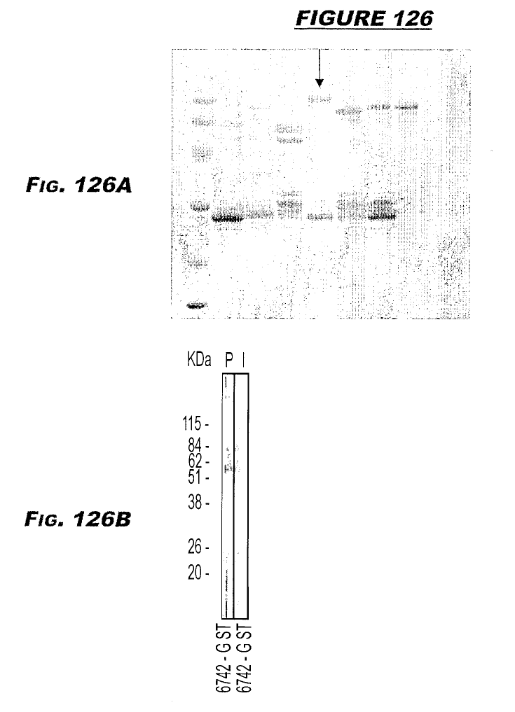

The protein was expressed in E. coli and purified as a GST-fusion product (FIG. 126A). The recombinant protein was used to immunize mice, whose sera were used in a Western blot (FIG. 126B) and for FACS analysis.

These experiments show that cp6742 is a surface-exposed and immunoaccessible protein, and that it is a useful immunogen. These properties are not evident from the sequence alone.

### Example 127

The following C. pneumoniae protein (PID 4376744) was expressed <SEQ ID 253; cp6744>:

| | |
|---|---|
| 1 | VIQHLLNFAL EETPSISVQY QEQEKLSPCD HSPEIGKKKR WNKLESFSTY |
| 51 | CSLFMSVKDH YKLNLGIQNS LSGWLLDPYR VCAPLSSPYS CPSYLLDLQN |
| 101 | KELRRSLLST FLDPKNLTSE TFRSVSINFG NSSFGQRWSE FLSRVLHDEK |
| 151 | EKHVAVVCND AKLLEEGLSP EALSLLEEDL RESGYSYLNI LSVSPEGVSK |
| 201 | VQERQILRRD LQGRSFTVMI TDLPLGSEDI RSLQLASDRI LVSSSLDAAD |
| 251 | ACASGCKVLV YENPNASWAQ ELENFYKQVE RRR* |

The cp6744 nucleotide sequence <SEQ ID 254> is:

| | |
|---|---|
| 1 | GTGATACAAC ATCTTCTAAA CTTTGCTCTA GAAGAGACCC CTTCCATTTC |
| 51 | CGTGCAATAC CAAGAACAAG AGAAGCTCTC TCCGTGCGAT CATTCCCCAG |
| 101 | AAATAGGTAA AAAGAAAAGA TGGAATAAGC TGGAATCCTT CTCCACGTAT |
| 151 | TGTTCTCTGT TTATGTCTGT TAAGGATCAT TATAAGCTGA ATCTAGGAAT |
| 201 | TCAGAATTCC CTGTCAGGGT GGCTTCTGGA TCCCTATAGG GTTTGCGCGC |
| 251 | CTTTATCTTC ACCGTACTCG TGTCCTTCCT ATCTTTTAGA TTTGCAAAAC |
| 301 | AAAGAGCTAC GTCGTTCCCT TCTGTCAACG TTTCTAGACC CTAAAAATCT |
| 351 | CACTAGCGAA ACATTCCGTT CTGTCTCTAT AAACTTTGGC AACTCTTCGT |
| 401 | TTGGACAGAG ATGGTCAGAG TTTCTATCTC GTGTTCTGCA CGACGAGAAA |
| 451 | GAAAAGCACG TAGCTGTTGT TTGTAATGAT GCAAAACTTC TGGAAGAAGG |
| 501 | ATTGTCCCCA GAGGCATTGT CTCTATTAGA AGAAGACTTA AGAGAATCAG |
| 551 | GGTATTCGTA TCTAAACATT CTCTCGGTGA GCCCCGAAGG AGTCTCCAAG |
| 601 | GTTCAGGAAC GTCAGATTCT AAGGCGAGAT CTCCAAGGAC GGTCCTTTAC |
| 651 | TGTCATGATT ACAGATCTTC CTTTAGGTAG CGAAGATATC CGTAGTTTAC |
| 701 | AATTAGCCTC GGATAGGATT TTAGTCTCCA GTTCTCTTGA TGCCGCGGAT |
| 751 | GCATGTGCTT CGGGATGTAA AGTCTTAGTC TACGAAAATC CAAATGCATC |
| 801 | CTGGGCTCAG GAATTGGAGA ACTTCTACAA ACAAGTTGAG AGAAGAAGGT |
| 851 | AG |

The PSORT algorithm predicts cytoplasm (0.3833).

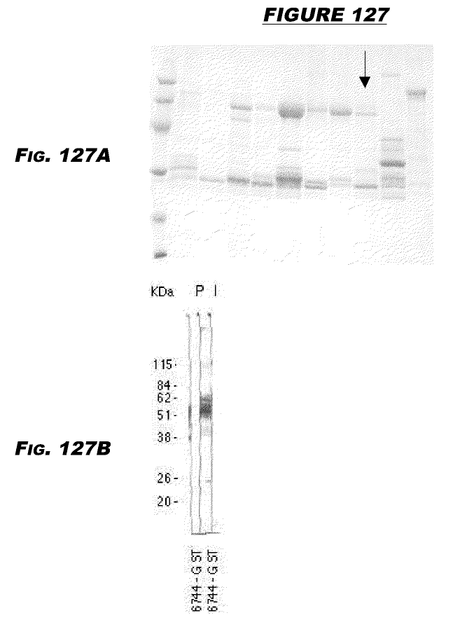

The protein was expressed in E. coli and purified as a GST-fusion product (FIG. 127A). The recombinant protein was used to immunize mice, whose sera were used in a Western blot (FIG. 127B) and for FACS analysis.

These experiments show that cp6744 is a surface-exposed and immunoaccessible protein, and that it is a useful immunogen. These properties are not evident from the sequence alone.

### Example 128

The following C. pneumoniae protein (PID 4376745) was expressed <SEQ ID 255; cp6745>:

| | |
|---|---|
| 1 | VACPSISSWF TVVRQHFVNA FDFTHPVCSR ITNFALGIIK AIPVLGHIVM |
| 51 | GIEWLISWIP RHTVRHGMFT SDVSSAIKVE QTRGHNCLAP LEAYLSSLRV |
| 101 | PISQEDLGKV HGRTPEDPFV DITPTEIVQL LPDEELSTVD EALQGVRSRL |
| 151 | TYAYRSVEKP MIQDLALVGF GLRDSADLIN FVRLANGVQN HYPHTKVKLY |
| 201 | LAKNLADVWD CEISEEEKGQ LRALGLDPKI ESISLTSAGL PSVPEVATVD |
| 251 | FMITCYGKDQ EVQDP* |

The cp6745 nucleotide sequence <SEQ ID 256> is:

```
    1 GTGGCTTGTC CAAGTATTTC TTCTTGGTTT ACTGTCGTTC GACAGCATTT
  51 TGTAAACGCC TTTGATTTCA CCCATCCCGT TTGTTCTCGG ATTACAAATT
101 TTGCTTTGGG GATCATTAAG GCAATTCCCG TATTAGGACA CATTGTCATG
151 GGAATCGAGT GGTTGATTTC CTGGATTCCC AGACACACCG TTCGTCATGG
201 AATGTTTACT TCTGATGTCT CTAGTGCTAT TAAAGTAGAA CAAACACGGG
251 GTCATAATTG TTTAGCTCCC CTAGAAGCCT ATTTAAGTAG CTTGAGAGTC
301 CCCATTTCCC AAGAAGATCT AGGCAAAGTA CACGGGAGAA CCCCAGAAGA
351 TCCCTTCGTA GATATCACAC CCACAGAAAT TGTCCAACTT CTCCCTGATG
401 AAGAACTCTC TACTGTAGAT GAGGCACTGC AAGGCGTTCG TAGTAGGTTA
451 ACCTATGCCT ATAGGTCCGT AGAGAAACCT ATGATTCAAG ATCTTGCTCT
501 TGTGGGTTTT GGTCTCCGAG ATTCTGCGGA CCTCATAAAT TTCGTGCGTC
551 TTGCTAATGG CGTGCAGAAT CACTATCCCC ATACTAAAGT GAAGCTCTAT
601 TTAGCGAAGA ACTTGGCAGA TGTCTGGGAC TGTGAAATTT CTGAAGAGGA
651 AAAAGGGCAA CTCCGAGCTC TAGGTTTAGA CCCTAAAATA GAGAGTATAT
701 CCCTTACGAG TGCAGGTCTT CCTTCAGTGC CAGAAGTCGC TACTGTCGAT
751 TTTATGATTA CCTGTTACGG GAAAGATCAG GAAGTCCAAG ATCCCTAG
```

The PSORT algorithm predicts inner membrane (0.2253).

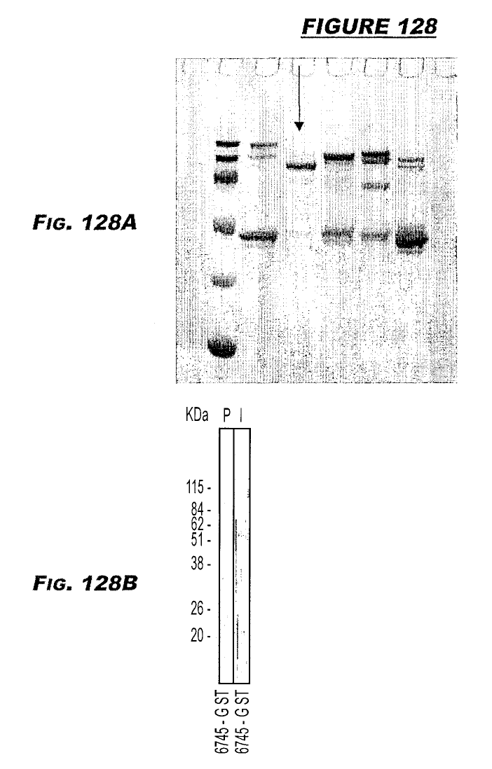

The protein was expressed in E. coli and purified as a GST-fusion product (FIG. 128A). The recombinant protein was used to immunize mice, whose sera were used in a Western blot (FIG. 128B) and for FACS analysis.

These experiments show that cp6745 is a surface-exposed and immunoaccessible protein, and that it is a useful immunogen. These properties are not evident from the sequence alone.

### Example 129

The following C. pneumoniae protein (PID 4376747) was expressed <SEQ ID 257; cp6747>:

| | |
|---|---|
| 1 | MMKQGVGQDA KELYTFLSRG NEHYQPCLWF SLEEELGFLF DEKMLCAPLS |
| 51 | EDHYCHSYLV DLVDQHLKDL ILSMFLDPQN ISAGELLKVS INVGDSFSPL |
| 101 | QQKDFLSMVL RDETGKNVVV VFKGVLSLPA TQVCKLVEEL NSKDYSYLNI |
| 151 | FSCHGDSSPQ LLFRKELEGT SGRYFTVICA LYLGDTDMRS LQLASERIMV |
| 201 | SREFDLVDAY AARCKLLKID HTNWRPGTFS RHADFADAVD VSAGFNSREF |
| 251 | KLITQANQGI LESGELPLPS KTFWEGFLAF CDRVTVTRHF IPMLDAAIKQ |
| 301 | AVWTHKHPSL IDKECEALDL KTQCLPSIVS YLEYVTNSHE KTSKGPFIQK |
| 351 | EIIADCSPLK EALFPGSDED VPSTSEDPSD DHPSDLEDS* |

The cp6747 nucleotide sequence <SEQ ID 258> is:

| | |
|---|---|
| 1 | ATGATGAAAC AAGGAGTCGG GCAGGATGCT AAAGAGCTAT ACACATTTCT |
| 51 | ATCTCGTGGG AATGAGCATT ACCAACCGTG TCTATGGTTC AGTCTCGAAG |
| 101 | AGGAACTCGG ATTCCTTTTC GATGAAAAAA TGCTCTGCGC CCCTCTATCT |
| 151 | GAGGATCACT ATTGCCACTC GTATCTTGTA GATCTAGTGG ATCAACATTT |
| 201 | AAAGGATTTA ATATTATCGA TGTTTTTAGA TCCTCAGAAT ATCTCAGCAG |
| 251 | GAGAACTCCT CAAGGTCTCT ATAAACGTTG GAGATTCTTT TTCTCCTCTA |
| 301 | CAACAGAAAG ATTTCCTCTC GATGGTCTTA CGTGATGAAA CGGGAAAAAA |
| 351 | CGTCGTCGTG GTTTTTAAAG GAGTTCTCTC CTTACCCGCA ACCCAAGTCT |
| 401 | GCAAATTAGT AGAGGAATTG AACTCTAAGG ACTACTCCTA CCTCAATATA |
| 451 | TTTTCTTGTC ACGGAGATAG TAGTCCTCAG CTTTTATTCC GTAAGGAATT |
| 501 | AGAGGGAACT TCAGGGCGTT ATTTTACAGT GATTTGCGCT TTATATCTAG |
| 551 | GGGATACAGA CATGCGTAGT TTACAACTTG CTTCTGAAAG GATCATGGTC |
| 601 | TCTAGAGAGT TTGATCTTGT AGATGCCTAT GCTGCAAGAT GCAAGCTCTT |
| 651 | GAAAATCGAT CATACAAATT GGAGACCTGG AACTTTCAGT CGCCACGCCG |
| 701 | ATTTCGCAGA TGCTGTAGAC GTATCAGCAG GATTTAACTC AAGAGAATTT |
| 751 | AAACTGATTA CGCAGGCGAA TCAAGGGATC CTAGAGTCTG GAGAACTCCC |
| 801 | GCTCCCTTCA AAAACCTTCT GGGAAGGATT CTTAGCATTC TGTGATCGAG |
| 851 | TGACTGTCAC GAGACACTTC ATTCCAATGT TAGACGCCGC TATAAAGCAA |
| 901 | GCGGTATGGA CTCATAAACA TCCCAGCTTG ATAGATAAAG AGTGTGAAGC |
| 951 | CCTAGACTTG AAAACACAGT GCTTGCCATC TATCGTATCG TACCTTGAAT |
| 1001 | ATGTCACAAA CTCTCACGAA AAAACATCGA AAGGCCCGTT CATACAAAAA |
| 1051 | GAGATTATCG CAGACTGTTC TCCTCTTAAA GAGGCGCTCT TCCCAGGTTC |
| 1101 | TGATGAAGAT GTTCCCTCTA CCTCTGAGGA TCCTTCAGAT GATCATCCTT |
| 1151 | CGGATCTTGA AGACTCTTAA |

The PSORT algorithm predicts inner membrane (0.1447).

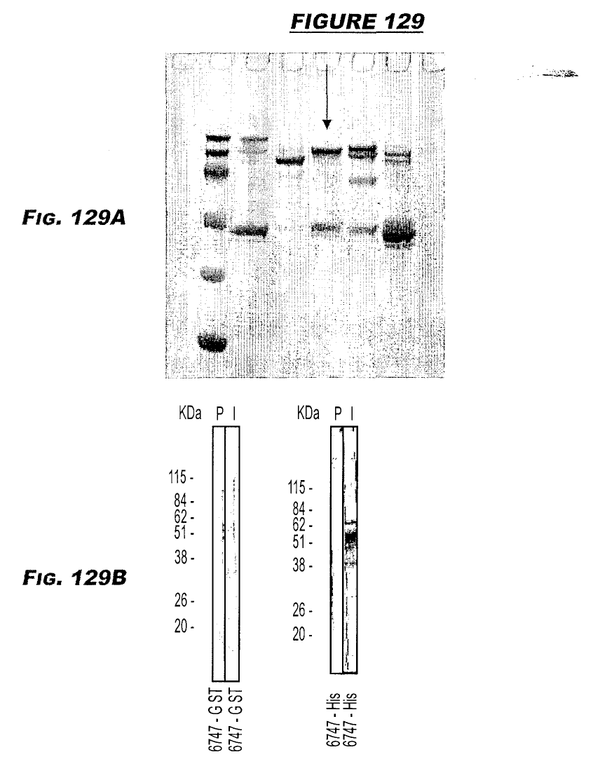

The protein was expressed in E. coli and purified as a GST-fusion product (FIG. 129A) and also as a his-tagged product. The recombinant proteins were used to immunize mice, whose sera were used in a Western blot (FIG. 129B) and for FACS analysis.

These experiments show that cp6747 is a surface-exposed and immunoaccessible protein, and that it is a useful immunogen. These properties are not evident from the sequence alone.

### Example 130

The following C. pneumoniae protein (PID 4376756) was expressed <SEQ ID 259; cp6756>:

| | |
|---|---|
| 1 | MASGIGGSSG LGKIPPKDNG DRSRSPSPKG ELGSHEISLP PQEHGEEGAS |
| 51 | GSSHIHSSSS FLPEDQESQS SSSAASSPGF FSRVRSGVDR ALKSFGNFFS |
| 101 | AESTSQARET RQAFVRLSKT ITADERRDVD SSSAAATEAR VAEDASVSGE |
| 151 | NPSQGVPETS SGPEPQRLFS LPSVKKQSGL GRLVQTVRDR IVLPSGAPPT |
| 201 | DSEPLSLYEL NLRLSSLRQE LSDIQSNDQL TPEEKAEATV TIQQLIQITE |
| 251 | FQCGYMEATQ SSVSLAEARF KGVETSDEIN SLCSELTDPE LQELMSDGDS |
| 301 | LQNLLDETAD DLEAALSHTR LSFSLDDNPT PIDNNPTLIS QEEPIYEEIG |
| 351 | GAADPQRTRE NWSTRLWNQI REALVSLLGM ILSILGSILH RLRIARHAAA |
| 401 | EAVGRCCTCR GEECTSSEED SMSVGSPSEI DETERTGSPH DVPRRNGSPR |
| 451 | EDSPLMNALV GWAHKHGAKT KESSESSTPE ISISAPIVRG WSQDSSVSFI |
| 501 | VMEDDHIFYD VPRRKDGIYD VPSSPRWSPA RELEEDVFGD YEVPITSAEP |
| 551 | SKDKNIYMTP RLATPAIYDL PSRPGSSGSS RSPSSDRVRS SSPNRRGVPL |
| 601 | PPVPSPAMSE EGSIYEDMSG ASGAGESDYE DMSRSPSPRG DLDEPIYANT |
| 651 | PEDNPFTQRN IDRILQERSG GASASEVEPI YDEIPWIHGR PPATLPRPEN |
| 701 | TLTNVSLRVS PGFGPEVRAA LLSESVSAVM VEAESIVPPT EPGDGESEYL |
| 751 | EPLGGLVATT KILLQKGWPR GESNA* |

The cp6756 nucleotide sequence <SEQ ID 260> is:

| | |
|---|---|
| 1 | ATGGCATCAG GAATCGGAGG ATCTAGTGGA TTAGGAAAGA TTCCACCTAA |
| 51 | AGATAATGGG GATAGAAGTC GATCGCCCTC TCCTAAGGGA GAACTTGGCA |
| 101 | GCCACGAGAT TTCCCTGCCT CCTCAAGAAC ATGGAGAGGA AGGAGCTTCA |
| 151 | GGATCTTCGC ATATACATAG CAGTTCCTCT TTTCTACCAG AAGATCAGGA |
| 201 | GTCTCAGAGC TCTTCTTCGG CAGCTTCTAG CCCGGGATTT TTTTCTCGCG |
| 251 | TACGTTCTGG GGTAGACAGG GCCTTAAAAT CATTTGGCAA CTTTTTTTCC |
| 301 | GCAGAGTCTA CGAGTCAAGC GCGTGAAACG CGACAAGCTT TTGTTAGATT |
| 351 | ATCAAAAACC ATCACCGCGG ATGAGAGACG GGATGTCGAT TCATCAAGTG |
| 401 | CTGCTGCTAC AGAAGCCCGA GTGGCAGAGG ACGCGAGTGT TTCAGGCGAA |
| 451 | AATCCTTCTC AGGGGGTTCC AGAAACCTCT TCTGGACCAG AACCTCAGCG |
| 501 | TTTATTTTCT CTTCCTTCAG TAAAAAAACA GAGCGGTTTG GGTCGGTTGG |
| 551 | TACAGACAGT TCGCGATCGC ATAGTACTTC CTAGTGGGGC TCCACCTACA |
| 601 | GACAGCGAGC CTTTAAGTCT CTACGAGCTA AACCTCCGTT TGAGTAGTTT |
| 651 | ACGTCAGGAG CTCTCTGACA TACAAAGTAA TGATCAGTTG ACTCCAGAGG |
| 701 | AAAAAGCAGA AGCCACAGTT ACCATACAAC AGCTGATCCA AATTACAGAA |
| 751 | TTCCAATGCG GCTATATGGA GGCAACACAA TCTTCGGTAT CTCTAGCAGA |
| 801 | AGCTCGTTTT AAGGGGGTAG AAACTAGTGA TGAGATCAAT TCCCTCTGTT |
| 851 | CAGAACTGAC AGATCCTGAG CTTCAAGAAC TCATGAGTGA TGGAGACTCT |
| 901 | CTTCAAAACC TATTAGATGA GACTGCCGAC GATTTAGAAG CTGCTTTGTC |
| 951 | CCATACTCGA TTGAGTTTTT CTTTAGACGA TAATCCAACT CCGATAGACA |
| 1001 | ATAATCCAAC TCTGATTTCT CAAGAAGAGC CTATTTATGA GGAAATCGGA |
| 1051 | GGAGCTGCAG ATCCTCAAAG AACTCGGGAA AACTGGTCTA CAAGATTATG |
| 1101 | GAATCAGATT CGCGAGGCTC TGGTTTCTCT TTTAGGAATG ATTTTAAGCA |
| 1151 | TTCTAGGGTC CATCTTGCAC AGGTTGCGTA TTGCTCGTCA TGCAGCTGCT |
| 1201 | GAAGCAGTGG GTCGTTGTTG CACGTGCCGA GGAGAAGAGT GTACTTCTTC |
| 1251 | TGAAGAGGAC TCGATGTCGG TGGGGTCTCC TTCAGAAATT GATGAAACTG |
| 1301 | AAAGAACGGG CTCTCCGCAT GACGTTCCAC GCAGAAATGG AAGTCCACGT |
| 1351 | GAAGATTCTC CATTGATGAA TGCCTTAGTA GGATGGGCAC ATAAGCACGG |
| 1401 | TGCTAAAACC AAGGAGAGTT CAGAATCAAG TACCCCGGAA ATTTCGATTT |
| 1451 | CTGCTCCCAT AGTGAGAGGT TGGAGTCAAG ACAGTTCCGT CAGTTTTATT |
| 1501 | GTTATGGAAG ATGATCATAT TTTCTATGAT GTTCCTCGTA GAAAAGATGG |
| 1551 | AATCTATGAC GTTCCTAGTT CCCCTAGATG GAGTCCTGCG CGAGAGTTGG |
| 1601 | AAGAGGATGT TTTTGGAGAT TATGAAGTTC CTATAACCTC TGCTGAACCA |
| 1651 | TCTAAAGACA AGAACATCTA CATGACACCT AGATTAGCAA CTCCTGCTAT |
| 1701 | CTATGATCTT CCTTCACGTC CAGGATCGTC TGGAAGCTCA CGTTCTCCGT |
| 1751 | CTTCAGATCG CGTACGAAGC AGCTCACCAA ATAGACGGGG TGTGCCTCTT |
| 1801 | CCTCCAGTTC CTTCACCTGC TATGAGTGAG GAGGGGAGCA TTTATGAGGA |
| 1851 | TATGAGCGGT GCTTCAGGTG CAGGTGAAAG TGATTATGAA GATATGAGCC |
| 1901 | GTTCCCCCTC TCCTAGAGGC GACTTGGATG AACCCATATA TGCTAATACT |
| 1951 | CCTGAAGATA ATCCATTTAC TCAGAGAAAT ATAGATAGAA TTTTACAGGA |
| 2001 | GAGGTCAGGC GGTGCTTCCG CTTCTCCTGT AGAGCCTATT TATGATGAGA |
| 2051 | TCCCATGGAT TCATGGCAGG CCCCCTGCTA CACTTCCAAG ACCCGAGAAT |
| 2101 | ACATTGACTA ATGTTTCGCT TAGAGTGAGC CCAGGGTTTG GACCAGAAGT |
| 2151 | AAGAGCCGCT TTGCTTAGCG AGAGCGTGAG TGCTGTTATG GTCGAAGCAG |
| 2201 | AGAGTATTGT TCCTCCAACA GAGCCGGGGG ACGGAGAATC AGAATATCTA |
| 2251 | GAGCCCTTAG GGGGACTTGT AGCTACAACG AAAATCTTAC TACAAAAAGG |
| 2301 | ATGGCCTCGT GGAGAGTCGA ATGCTTAG |

The PSORT algorithm predicts inner membrane (0.3994).

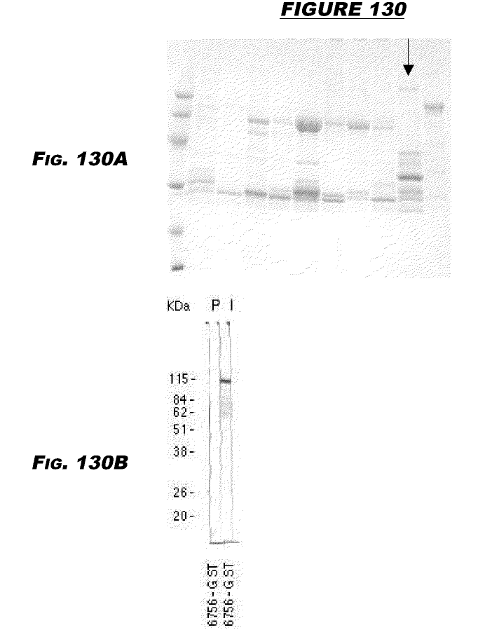

The protein was expressed in E. coli and purified as a GST-fusion product (FIG. 130A). The recombinant protein was used to immunize mice, whose sera were used in a Western blot (FIG. 130B) and for FACS analysis.

These experiments show that cp6756 is a surface-exposed and immunoaccessible protein, and that it is a useful immunogen. These properties are not evident from the sequence alone.

### Example 131

The following C. pneumoniae protein (PID 4376761) was expressed <SEQ ID 261; cp6761>:

| | |
|---|---|
| 1 | MTVAEVKGTF KLVCLGCRVN QYEVQAYRDQ LTILGYQEVL DSEIPADLCI |
| 51 | INTCAVTASA ESSGRHAVRQ LCRQNPTAHI VVTGCLGESD KEFFASLDRQ |
| 101 | CTLVSNKEKS RLIEKIFSYD TTFPEFKIHS FEGKSRAFIK VQDGCNSFCS |
| 151 | YCIIPYLRGR SVSRPAEKIL AEIAGVVDQG YREVVIAGIN VGDYCDGERS |
| 201 | LASLIEQVDR IPGIERIRIS SIDPDDITED LHRAITSSRH TCPSSHLVLQ |
| 251 | SGSNSILKRM NRKYSRGDFL DCVEKFRASD PRYAFTTDVI VGFPGESDQD |
| 301 | FEDTLRIIED VGFIKVHSFP FSARRRTKAY TFDNQIPNQV IYERKKYLAE |
| 351 | VAKRVGQKEM MKRLGETTEV LVEKVTGQVA TGHSPYFEKV SFPVVGTVAI |
| 401 | NTLVSVRLDR VEEEGLIGEI V* |

The cp6761 nucleotide sequence <SEQ ID 262> is:

| | |
|---|---|
| 1 | ATGACGGTTG CGGAAGTCAA AGGAACATTT AAGCTGGTCT GTTTAGGCTG |
| 51 | TCGGGTGAAT CAGTATGAGG TCCAAGCATA TCGCGACCAG TTGACTATCT |
| 101 | TAGGTTACCA AGAGGTCCTG GATTCTGAAA TCCCTGCAGA TTTATGCATA |
| 151 | ATCAATACGT GTGCTGTCAC AGCTTCTGCT GAGAGTTCGG GTCGTCATGC |
| 201 | TGTGCGTCAG TTATGTCGTC AGAACCCTAC AGCACATATT GTTGTCACAG |
| 251 | GTTGTTTGGG GGAATCTGAC AAAGAGTTTT TTGCTTCTTT GGATCGGCAA |
| 301 | TGCACACTTG TTTCCAATAA AGAAAAATCC CGACTTATAG AAAAAATTTT |
| 351 | TTCCTATGAT ACGACCTTCC CTGAGTTCAA GATCCATAGT TTTGAGGGAA |
| 401 | AGTCTCGAGC TTTTATTAAA GTTCAAGATG GCTGTAATTC TTTTTGCTCG |
| 451 | TACTGCATTA TTCCTTATTT GCGGGGGCGT TCGGTTTCTC GTCCTGCTGA |
| 501 | GAAGATTTTA GCTGAAATCG CAGGGGTTGT AGACCAAGGA TATCGCGAAG |
| 551 | TTGTAATTGC AGGAATTAAT GTTGGAGATT ATTGCGATGG AGAGCGTTCA |
| 601 | TTAGCCTCTT TGATTGAACA GGTGGACCGG ATTCCTGGAA TTGAGAGGAT |
| 651 | TCGAATTTCC TCTATAGATC CTGATGATAT CACTGAAGAT CTGCACCGTG |
| 701 | CCATCACCTC ATCGCGTCAC ACTTGTCCTT CGTCACACCT TGTTCTTCAA |
| 751 | TCGGGGTCGA ATTCAATTTT AAAGAGAATG AACCGGAAGT ATTCTCGCGG |
| 801 | AGATTTTTTA GATTGTGTAG AGAAGTTCCG TGCTTCTGAT CCTCGCTATG |
| 851 | CCTTTACTAC AGATGTGATT GTCGGATTTC CTGGAGAGAG TGATCAAGAT |
| 901 | TTTGAAGATA CTTTGAGAAT TATTGAAGAT GTAGGCTTTA TTAAAGTGCA |
| 951 | TAGTTTCCCT TTCAGTGCTC GTCGTCGTAC TAAGGCATAT ACTTTTGATA |
| 1001 | ATCAGATTCC CAATCAGGTG ATCTATGAGA GGAAGAAGTA TCTTGCTGAG |
| 1051 | GTTGCTAAGA GGGTAGGCCA GAAAGAGATG ATGAAGCGTT TAGGAGAGAC |
| 1101 | TACAGAGGTG CTTGTTGAGA AAGTAACGGG GCAGGTTGCT ACGGGTCACT |
| 1151 | CTCCTTATTT TGAAAAGGTT TCTTTCCCTG TTGTAGGAAC GGTAGCTATC |
| 1201 | AACACTCTAG TTTCTGTGCG TCTTGATAGG GTAGAGGAAG AAGGGCTGAT |
| 1251 | TGGGGAGATT GTATGA |

The PSORT algorithm predicts inner membrane (0.1574).

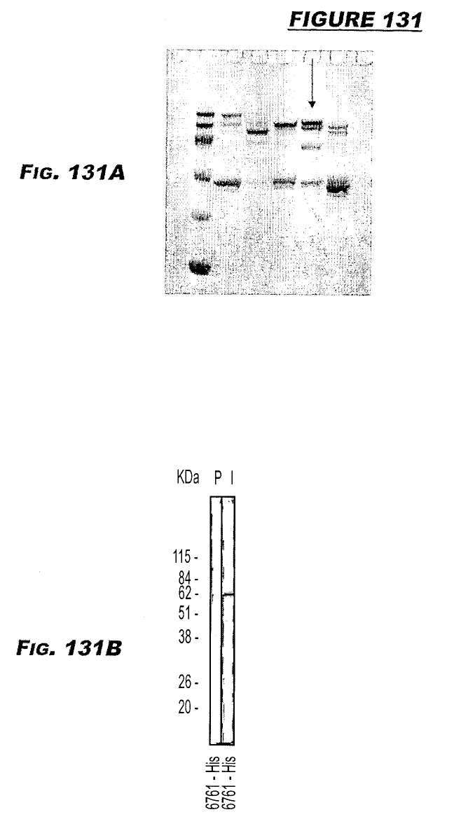

The protein was expressed in E. coli and purified as a GST-fusion product (FIG. 131A) and also as a his-tagged product. The recombinant proteins were used to immunize mice, whose sera were used in a Western blot (FIG. 131B) and for FACS analysis.

These experiments show that cp6761 is a surface-exposed and immunoaccessible protein, and that it is a useful immunogen. These properties are not evident from the sequence alone.

### Example 132

The following C. pneumoniae protein (PID 4376766) was expressed <SEQ ID 263; cp6766>:

| | |
|---|---|
| 1 | MATSVPVTSS TSVGEANSSN ERFTERTSRM YYAALVLGAL SCLIFIAMIV |
| 51 | IFPQVGLWAV VLGFALGCLL LSLAIVFAVS GLVLGKTLEP SREATPPEIV |
| 101 | AQKEWTTQQD VLGNEYWRSE LISLFLRGDL HESLIVDSKD RSLDIDQSLQ |
| 151 | NILKLEPLST TLSLLKKDCV HINIILHLVR QWNLLGVDLS PEVTAHAEEL |
| 201 | LLFLIEEQYY SPDILKLIRY GDALQATSPL MDWADSGSFS VDADGVFSCR |
| 251 | REECSPEDAL AQFDLLLALE NPDRRFLKDS FLTYIWSSSF FEKFLHRHLE |
| 301 | SLQRKLPETA IDVARYEAQI QTFLSRYFQK LDLINAMSLD WGYNCAEGEK |
| 351 | CYESANQRLD NLFIAFSSSV RAMKRLFDKY GSVVRVDRRQ IREQILSNTE |
| 401 | ILENESGFLC SLYEYPLSYL IDWAVLLDCV RGTEISLEDQ ADYTVCLQGL |
| 451 | DSMLSQFASR LQSGQKVLNP RDVLSEQAAV MLVHGLAAQG VSFQGLKALM |
| 501 | YLTAVPQRMW LGALPLFESF PVFNRMKEFL GESLGD* |

The cp6766 nucleotide sequence <SEQ ID 264> is:

| | |
|---|---|
| 1 | ATGGCAACCT CTGTTCCTGT AACTTCATCT ACTTCTGTAG GAGAGGCTAA |
| 51 | CTCCTCCAAC GAAAGATTTA CTGAACGAAC ATCGCGAATG TATTACGCAG |
| 101 | CTTTAGTCCT AGGGGCTTTG AGCTGTTTAA TTTTTATTGC TATGATTGTC |
| 151 | ATTTTCCCAC AGGTCGGATT GTGGGCTGTG GTCCTCGGGT TTGCTCTTGG |
| 201 | ATGTTTACTT TTAAGCTTAG CTATCGTTTT TGCTGTCTCC GGTCTCGTTT |
| 251 | TAGGCAAGAC TTTAGAACCT AGTCGAGAAG CGACTCCTCC AGAAATTGTT |
| 301 | GCGCAAAAGG AGTGGACTAC ACAACAAGAT GTCTTAGGGA ATGAGTATTG |
| 351 | GCGTTCCGAG TTGATTTCCT TGTTCTTACG AGGGGATCTC CACGAATCTC |
| 401 | TGATTGTTGA TTCTAAGGAT CGATCTTTAG ATATTGATCA GAGTTTACAA |
| 451 | AATATATTGA AACTTGAGCC CCTATCTACG ACACTTTCGC TGTTAAAGAA |
| 501 | AGATTGTGTC CACATCAATA TCATTTTACA TTTAGTGAGA CAGTGGAACT |
| 551 | TACTGGGAGT GGATCTTAGT CCTGAAGTCA CTGCGCACGC CGAGGAACTT |
| 601 | CTACTCTTTT TGATAGAAGA GCAGTATTAC TCTCCTGATA TTTTGAAATT |
| 651 | GATTCGCTAC GGAGATGCTT TACAAGCAAC GTCTCCTTTG ATGGATTGGG |
| 701 | CAGATTCAGG TTCCTTTAGT GTAGACGCAG ACGGGGTATT TAGCTGTCGC |
| 751 | AGAGAAGAAT GTTCTCCTGA GGATGCTTTG GCGCAATTCG ATCTTCTTTT |
| 801 | GGCGTTGGAA AATCCCGACA GACGCTTCTT AAAGGATTCT TTTCTTACCT |
| 851 | ACATTTGGTC GTCTTCATTT TTTGAGAAGT TTTTACATCG CCATCTAGAG |
| 901 | AGCTTGCAAA GAAAGCTCCC AGAGACAGCG ATCGATGTCG CCCGCTATGA |
| 951 | AGCACAAATA CAAACATTTC TCTCTCGCTA TTTTCAGAAG CTCGATTTGA |
| 1001 | TAAACGCAAT GTCCTTAGAT TGGGGATATA ACTGTGCTGA GGGAGAAAAA |
| 1051 | TGTTATGAGA GCGCAAATCA AAGATTAGAC AACCTATTTA TTGCTTTTTC |
| 1101 | TTCTTCTGTT CCTGCTATGA AGCGGCTCTT TGACAAATAT GGTTCTGTGG |
| 1151 | TACGGGTAGA TCGTAGGCAG ATTCGTGAGC AGATTCTTTC GAACACTGAA |
| 1201 | ATCTTAGAAA ATGAGTCAGG GTTCCTCTGC AGTTTGTATG AATATCCTTT |
| 1251 | ATCCTATTTG ATAGATTGGG CTGTTTTGCT AGACTGTGTT CGCGGTACCG |
| 1301 | AAATCTCTCT AGAAGATCAG GCCGATTACA CCGTTTGTTT GCAAGGCTTG |
| 1351 | GATTCTATGT TATCTCAATT TGCGAGTCGT TTACAGTCTG GACAAAAAGT |
| 1401 | ATTGAATCCT AGAGATGTTT TAAGTGAACA GGCTGCGGTT ATGCTTGTTC |
| 1451 | ATGGCTTGGC AGCACAGGGC GTGTCGTTTC AAGGATTGAA AGCTTTGATG |
| 1501 | TATTTGACAG CCGTTCCCCA AAGAATGTGG TTAGGAGCAT TGCCTTTATT |
| 1551 | TGAATCTTTT CCTGTCTTTA ATCGGATGAA AGAATTTCTT GGGGAATCTC |
| 1601 | TGGGAGACTA G |

The PSORT algorithm predicts inner membrane (0.6158).

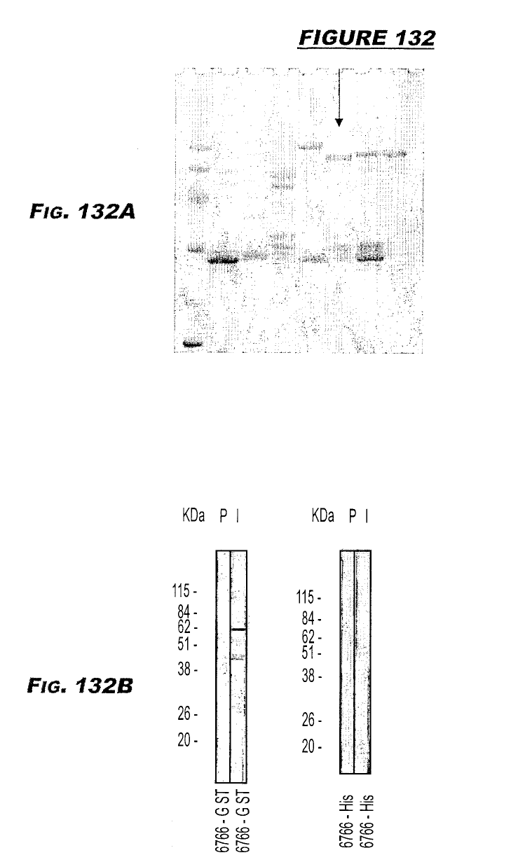

The protein was expressed in E. coli and purified as a GST-fusion product (FIG. 132A) and also as a his-tagged product. The recombinant proteins were used to immunize mice, whose sera were used in a Western blot (FIG. 132B) and for FACS analysis.

These experiments show that cp6766 is a surface-exposed and immunoaccessible protein, and that it is a useful immunogen. These properties are not evident from the sequence alone.

### Example 133

The following C. pneumoniae protein (PID 4376804) was expressed <SEQ ID 265; cp6804>:

| | |
|---|---|
| 1 | MSNQLQPCIS LGCVSYINSF PLSLQLIKRN DIRCVLAPPA DLLNLLIEGK |
| 51 | LDVALTSSLG AISHNLGYVP GFGIAANQRI LSVNLYAAPT FFNSPQPRIA |
| 101 | ATLESRSSIG LLKVLCRHLW RIPTPHILRF ITTKVLRQTP ENYDGLLLIG |
| 151 | DAALQHPVLP GFVTYDLASG WYDLTKLPFV FALLLHSTSW KEHPLPNLAM |
| 201 | EEALQQFESS PEEVLKEAHQ HTGLPPSLLQ EYYALCQYRL GEEHYESFEK |
| 251 | FREYYGTLYQ QARL |

The cp6804 nucleotide sequence <SEQ ID 266> is:

| | |
|---|---|
| 1 | ATGTCTAACC AACTCCAGCC ATGTATAAGC TTAGGCTGCG TAAGTTATAT |
| 51 | TAATTCCTTT CCGCTGTCCC TACAACTCAT AAAAAGAAAC GATATTCGCT |
| 101 | GTGTTCTTGC TCCCCCTGCA GACCTCCTCA ACTTGCTAAT CGAAGGGAAA |
| 151 | CTCGATGTTG CTTTGACCTC ATCCCTAGGA GCTATCTCTC ATAACTTGGG |
| 201 | GTATGTCCCC GGCTTTGGAA TTGCAGCAAA CCAACGTATC CTCAGTGTAA |
| 251 | ACCTCTATGC AGCTCCCACT TTCTTTAACT CACCGCAACC TCGGATTGCC |
| 301 | GCAACTTTAG AAAGTCGCTC CTCTATAGGA CTCTTAAAAG TGCTTTGTCG |
| 351 | TCATCTCTGG CGCATCCCAA CTCCTCATAT CCTAAGATTC ATAACTACAA |
| 401 | AAGTACTCAG ACAAACCCCT GAAAATTATG ATGGCCTCCT CCTAATCGGA |
| 451 | GATGCAGCGC TACAACATCC TGTACTTCCT GGATTTGTAA CCTATGACCT |
| 501 | TGCCTCGGGG TGGTATGATC TTACAAAGCT ACCTTTTGTA TTTGCTCTTC |
| 551 | TTCTACACAG CACCTCTTGG AAAGAACATC CCCTACCCAA CCTTGCGATG |
| 601 | GAAGAAGCCC TCCAACAGTT CGAATCTTCA CCCGAAGAAG TCCTTAAAGA |
| 651 | AGCTCATCAA CATACAGGTC TGCCCCCTTC TCTTCTTCAA GAATACTATG |
| 701 | CCCTATGCCA GTACCGTCTA GGAGAAGAAC ACTACGAAAG CTTTGAAAAA |
| 751 | TTCCGGGAAT ATTATGGAAC CCTCTACCAA CAAGCCCGAC TGTAA |

The PSORT algorithm predicts inner membrane (0.060).

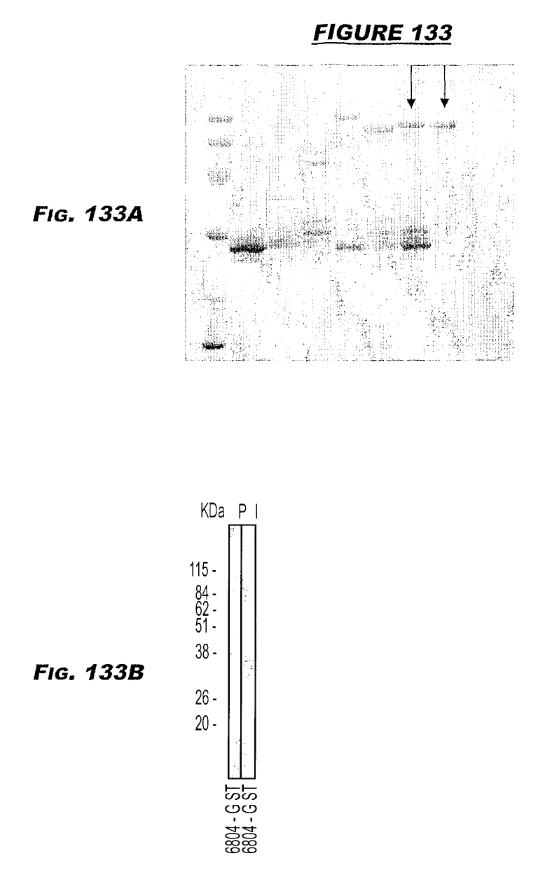

The protein was expressed in E. coli and purified as a GST-fusion product (FIG. 133A). The recombinant protein was used to immunize mice, whose sera were used in a Western blot (FIG. 133B) and for FACS analysis.

These experiments show that cp6804 is a surface-exposed and immunoaccessible protein, and that it is a useful immunogen. These properties are not evident from the sequence alone.

### Example 134

The following C. pneumoniae protein (PID 4376805) was expressed <SEQ ID 267; cp6805>:

| | |
|---|---|
| 1 | MSSLLSCGRI EPTRVTCSLK TYLEDTSQNQ LSTRLVRASV IFLCALLIIL |
| 51 | VCVALSSLIP SIMALATSFT VMGLILFVMS LLGDVAIISY LTYSTVTSYR |
| 101 | QNKRAFEIHK PARSVYYEGV RHWDLGRSSL GTGEIPIVRT LFSPFQNHGL |
| 151 | NHALAAKIFL FMEHFSPEPP NEPLVDWACL IRDFRPHVSS LCFVIEKQGS |
| 201 | SLRTKEGNTI CEAFRSDYDA HFAMVDCYRL IHSKLIIEKM GLKNIDIIPS |
| 251 | VMVREDYPSR PGEGYREGLL RMYGGKGAL* |

The cp6805 nucleotide sequence <SEQ ID 268> is:

| | |
|---|---|
| 1 | ATGTCATCAC TACTGAGCTG CGGAAGAATA GAGCCGACTC GGGTTACCTG |
| 51 | TAGCTTAAAG ACGTATCTTG AGGATACGAG TCAGAATCAG TTGAGCACAC |
| 101 | GTCTAGTTCG GGCAAGTGTC ATCTTTTTAT GCGCATTGTT GATCATTTTG |
| 151 | GTTTGTGTGG CCCTCTCTAG TTTGATTCCA AGCATTATGG CCTTGGCGAC |
| 201 | CTCTTTTACG GTAATGGGGT TAATTCTTTT TGTGATGTCA CTTCTTGGTG |
| 251 | ACGTTGCAAT TATAAGTTAT CTTACTTATA GCACTGTTAC GAGTTACCGG |
| 301 | CAAAATAAGA GAGCTTTTGA GATTCACAAG CCCGCTCGCT CCGTTTACTA |
| 351 | CGAGGGGGTC CGCCATTGGG ATTTAGGACG ATCATCTTTA GGCACAGGCG |
| 401 | AGATTCCTAT AGTAAGGACG TTATTCTCTC CATTTCAGAA CCATGGTCTT |
| 451 | AACCATGCCT TAGCTGCTAA AATTTTCCTA TTTATGGAGC ATTTCAGCCC |
| 501 | TGAGCCACCG AACGAGCCTT TGGTGGATTG GGCCTGTTTG ATTCGGGATT |
| 551 | TTAGGCCTCA CGTCAGTTCT TTGTGCTTTG TTATTGAAAA ACAAGGGTCA |
| 601 | TCGCTGAGGA CTAAGGAAGG CAATACGATT TGTGAGGCTT TCCGCTCTGA |
| 651 | TTACGACGCC CATTTTGCTA TGGTAGATTG CTACCGGTTG ATCCACTCTA |
| 701 | AGTTGATTAT AGAGAAAATG GGATTGAAGA ATATCGATAT CATTCCGAGT |
| 751 | GTCATGGTTC GTGAAGATTA TCCTAGCCGT CCTGGGGAGG GCTATCGCGA |
| 801 | AGGCCTATTA CGTATGTATG GTGGCAAGGG GGCTCTGTGA |

The PSORT algorithm predicts inner membrane (0.711).

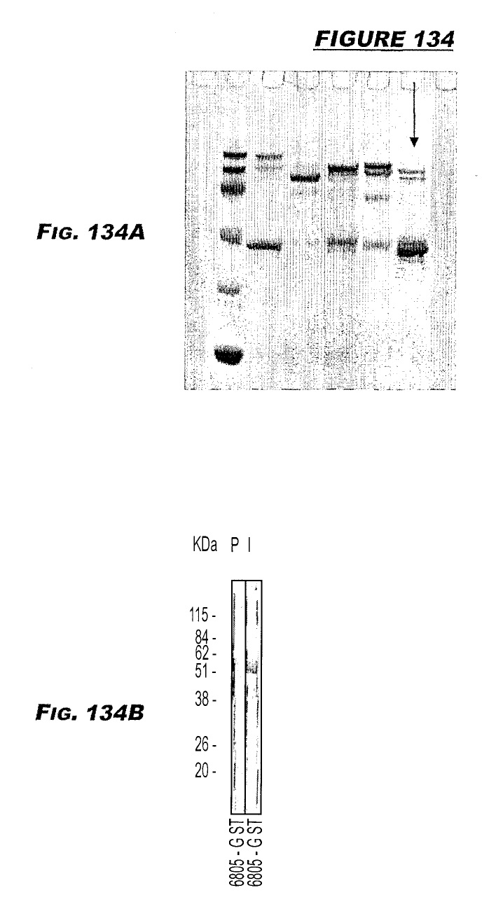

The protein was expressed in E. coli and purified as a GST-fusion product (FIG. 134A). The recombinant protein was used to immunize mice, whose sera were used in a Western blot (FIG. 134B) and for FACS analysis.

These experiments show that cp6805 is a surface-exposed and immunoaccessible protein, and that it is a useful immunogen. These properties are not evident from the sequence alone.

### Example 135

The following C. pneumoniae protein (PID 4376813) was expressed <SEQ ID 269; cp6813>:

| | |
|---|---|
| 1 | MSGPSRTESS QVSVLSYVPR DKEIAPKKQF TIAKISTLAI LASLALGALV |
| 51 | AGISLTIVLG NPVFLALLIT TALFSVVTFL VYHQMTSKVS SNWQKVLEQN |
| 101 | FKPLGKAWQE KNVDCYSNEM QFYNNHLNPK FKVAIQTDAS QPFQPTFLTG |
| 151 | LRVIEKNQST GIIFNPVGPT NLIDNTATNL STILYSTLKD KSVWDTCKQR |
| 201 | EGGPAKGEDP FSPTEVRVVK LPNEALDQTF NLNLSSAEKK SILPTFLGHV |
| 251 | CGPKSEELPN QQEYYRQALL AYENCLKAAI ESHAAIVALP LFTSVYEVPP |
| 301 | EEILPKEGTF YWDNQTQAFC KRALLDAIQN TALRYPQRSL LVILQDPFNT |
| 351 | IESQSRSEE* |

The cp6813 nucleotide sequence <SEQ ID 270> is:

| | |
|---|---|
| 1 | ATGTCAGGAC CCTCACGTAC TGAGAGCTCT CAAGTTTCTG TACTATCCTA |
| 51 | TGTGCCTCGG GATAAAGAAA TTGCTCCTAA AAAACAGTTT ACCATAGCAA |
| 101 | AAATATCCAC TCTTGCAATC CTAGCTTCTT TAGCTTTAGG AGCTTTGGTG |
| 151 | GCTGGAATCT CTTTAACGAT AGTATTAGGG AACCCTGTAT TTTTGGCTCT |
| 201 | TCTCATTACC ACGGCCCTCT TCTCAGTTGT AACCTTCTTA GTCTACCACC |
| 251 | AAATGACCTC AAAGGTATCT TCTAACTGGC AGAAAGTTCT AGAGCAAAAC |
| 301 | TTCAAGCCTT TGGGAAAAGC GTGGCAAGAA AAAAACGTAG ACTGCTACTC |
| 351 | AAACGAGATG CAATTTTACA ATAATCACCT GAACCCTAAG TTCAAGGTAG |
| 401 | CGATACAAAC AGATGCGTCT CAACCATTTC AGCCTACTTT CTTAACTGGA |
| 451 | CTTAGAGTGA TCGAAAAAAA TCAATCCACA GGGATCATCT TTAATCCCGT |
| 501 | AGGCCCAACG AATCTGATCG ACAACACTGC AACGAACCTC TCTACTATCC |
| 551 | TTTACTCCAC CCTAAAAGAT AAAAGCGTGT GGGATACATG CAAGCAACGC |
| 601 | GAAGGGGGTC CCGCAAAAGG AGAAGACCCC TTTTCCCCTA CCGAAGTGAG |
| 651 | AGTAGTAAAA CTTCCAAACG AAGCTCTAGA TCAAACGTTT AATCTAAATT |
| 701 | TAAGCTCTGC AGAAAAGAAA AGTATTCTTC CGACCTTTTT AGGCCACGTA |
| 751 | TGCGGCCCTA AATCTGAAGA GTTACCAAAT CAGCAAGAAT ATTATCGCCA |
| 801 | AGCTTTACTA GCGTACGAGA ACTGCCTTAA AGCAGCTATA GAAAGTCATG |
| 851 | CAGCAATCGT TGCTCTTCCT CTCTTTACTT CGGTCTATGA AGTGCCTCCA |
| 901 | GAAGAGATTC TTCCTAAAGA AGGCACTTTC TATTGGGACA ACCAAACTCA |
| 951 | AGCGTTTTGC AAACGCGCTT TATTGGACGC TATTCAAAAT ACGGCCCTAC |
| 1001 | GCTATCCTCA AAGATCTTTA CTTGTTATAC TCCAAGATCC TTTTAATACT |
| 1051 | ATAGAATCAC AAAGTCGTTC TGAGGAGTAA |

The PSORT algorithm predicts inner membrane (0.4291).

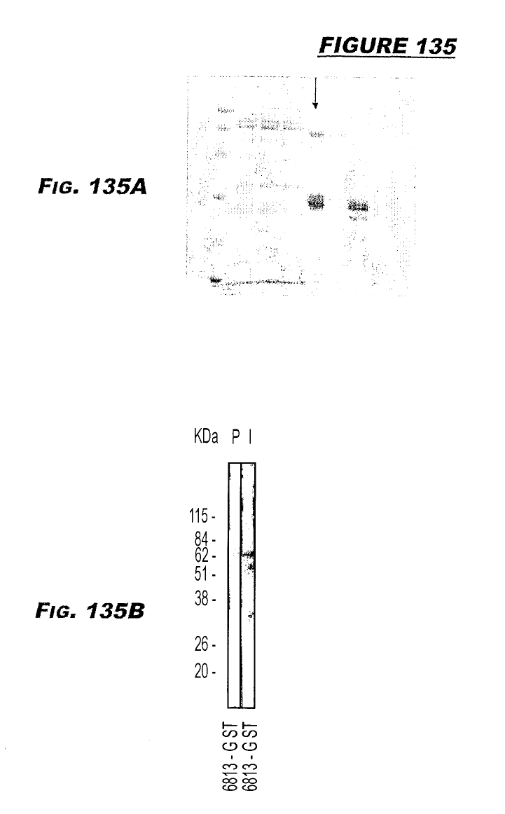

The protein was expressed in E. coli and purified as a GST-fusion product (FIG. 135A). The recombinant protein was used to immunize mice, whose sera were used in a Western blot (FIG. 135B) and for FACS analysis.

These experiments show that cp6813 is a surface-exposed and immunoaccessible protein, and that it is a useful immunogen. These properties are not evident from the sequence alone.

### Example 136

The following C. pneumoniae protein (PID 4376844) was expressed <SEQ ID 271; cp6844>:

| | |
|---|---|
| 1 | MWRVVLRFLI IFILGRAVFP LPASESFSWE TSTCLTVLGI PFIDIILTTN |
| 51 | EDFVAQCGLQ IGTISSTNNA KIKEIFLIYK EKFPEASISF KRKEPLNLSQ |
| 101 | SHLSDLGILC MRNGETYAEG MANKENGPAL KQPKDLRLVL RCPNQPDTLL |
| 151 | YSEKEAEKGI ETNTCLCNQG YTLLDGQLIL YGDSIEKFLK ETKRKNNHTL |
| 201 | VDLCDSQVVT TFLGRFWSLL NYVQVLFLSE DSAKILAGIP DLAQATQLLS |
| 251 | HTVPLLFIYT NDSIHIIEQG KESSFTYNQD LTEPILGFLF GYINRGSMEY |
| 301 | CFNCAQSSLG ET* |

The cp6844 nucleotide sequence <SEQ ID 272> is:

| | |
|---|---|
| 1 | ATGTGGCGCG TTGTCCTCAG ATTCCTTATA ATTTTTATCT TGGGAAGAGC |
| 51 | CGTCTTCCCT CTAAGAGCTT CAGAAAGCTT CTCCTGGGAA ACATCGACCT |
| 101 | GTTTAACAGT GCTAGGGATT CCTTTCATAG ATATTATCCT CACAACGAAT |
| 151 | GAGGACTTTG TTGCCCAGTG CGGCCTGCAA ATAGGAACCA TTTCTTCGAC |
| 201 | TAATAACGCA AAAATAAAAG AAATTTTTTT GATATATAAG GAAAAATTTC |
| 251 | CAGAAGCCTC TATCAGTTTC AAACGAAAAG AACCTCTAAA CCTTTCCCAA |
| 301 | TCCCATCTCT CCGATTTAGG TATTTTATGT ATGCGTAACG GAGAAACTTA |
| 351 | CGCTGAGGGA ATGGCAAATA AAGAAAACGG ACCCGCTCTA AAACAACCCA |
| 401 | AGGATCTAAG ATTAGTTTTA CGTTGTCCTA ACCAACCAGA TACCCTGCTC |
| 451 | TACTCGGAAA AAGAAGCAGA AAAGGGCATA GAAACAAATA CTTGCCTATG |
| 501 | CAATCAGGGA TACACACTCC TGGATGGGCA ATTGATTCTC TACGGGGATA |
| 551 | GTATAGAAAA GTTTCTGAAA GAGACCAAAA GAAAGAATAA CCACACGCTT |
| 601 | GTTGATCTTT GTGACTCACA AGTCGTGACC ACGTTCCTCG GTCGCTTTTG |
| 651 | GTCTCTTCTA AACTACGTTC AAGTTCTTTT CCTATCTGAA GACTCCGCTA |
| 701 | AAATTCTTGC GGGCATCCCA GACCTAGCTC AAGCTACGCA ATTGCTTTCC |
| 751 | CACACCGTAC CTTTGCTTTT TATTTATACC AACGATTCTA TTCACATCAT |
| 801 | AGAACAAGGC AAAGAAAGTA GTTTTACCTA TAACCAAGAT TTAACAGAGC |
| 851 | CCATTTTAGG ATTTCTCTTT GGTTACATAA ATCGCGGCTC TATGGAATAC |
| 901 | TGCTTTAATT GTGCACAGTC TTCATTAGGA GAAACCTAA |

The PSORT algorithm predicts inner membrane (0.1786).

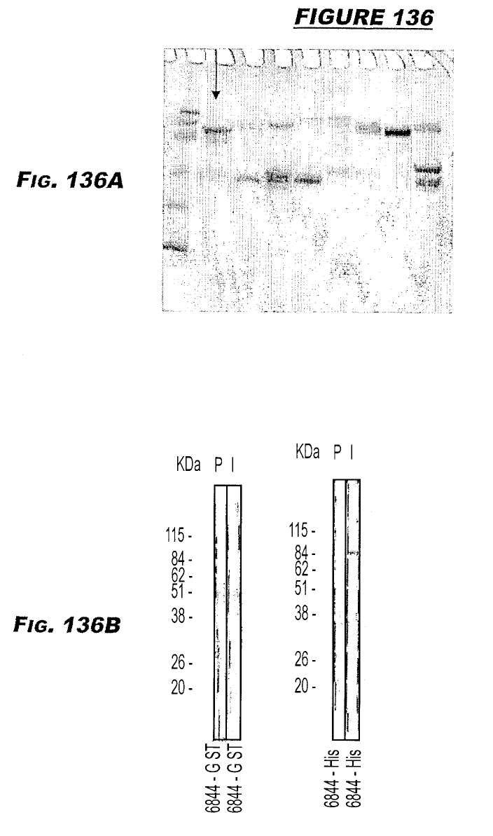

The protein was expressed in E. coli and purified as a GST-fusion product (FIG. 136A) and also in his-tagged form. The recombinant proteins were used to immunize mice, whose sera were used in a Western blot (FIG. 136B) and for FACS analysis.

These experiments show that cp6844 is a surface-exposed and immunoaccessible protein, and that it is a useful immunogen. These properties are not evident from the sequence alone.

### Example 137

The following C. pneumoniae protein (PID 4377201) was expressed <SEQ ID 273; cp7201>:

| | |
|---|---|
| 1 | VLVGICPSLY PEHPRSFYYR VSGDIGSRFD DRGFVNSGVE TLPYSSGSFG |
| 51 | IFWISFTDPT FNFAIVNTFM RTAGINEVSR PMTQDTETSL IEMRDLSEQQ |
| 101 | EANNTDSLEQ EESLMGIVGH TVGGVSMTVT SSPNIFYRIQ TLLGLPETLA |
| 151 | EAEENPTFPN STIDSLAEIM MNLVRISDAV SIFWIFPIVD TTYNGVLLAV |
| 201 | CIGFFGINGI CSTFLMLTNP RSRRDRWRNL RIMVLCYRSL GSGMNLFDLS |
| 251 | NNVRMAARRH VTSCTVALYA MVTLFGWTVA IQDALQYGFP SVRDAFYRYC |
| 301 | LRHRYCLTQR NEDSLQTTGT RFQVTRTHLE DQQMVASILN LSVFGLFFGF |
| 351 | VGLMTTFGGL ETSPSCRWDA ANNRTVGIF* |

The cp7201 nucleotide sequence <SEQ ID 274> is:

| | |
|---|---|
| 1 | GTGCTCGTTG GTATCTGTCC TTCTCTATAT CCAGAACATC CTCGCTCCTT |
| 51 | TTATTATCGT GTTTCTGGAG ATATAGGCTC CCGATTCGAC GATAGAGGAT |
| 101 | TTGTAAACTC TGGAGTCGAA ACCCTGCCAT ACTCTTCAGG CAGCTTTGGG |
| 151 | ATTTTTTGGA TCTCGTTTAC GGATCCCACA TTTAATTTTG CTATCGTAAA |
| 201 | TACCTTTATG CGAACTGCAG GGATCAATGA AGTCTCTAGA CCCATGACAC |
| 251 | AAGATACAGA AACTTCATTG ATAGAAATGA GAGACCTAAG TGAACAACAA |
| 301 | GAAGCGAATA ACACAGATTC TTTAGAGCAA GAAGAGAGCT TAATGGGTAT |
| 351 | TGTAGGACAT ACTGTGGGAG GAGTTTCCAT GACCGTGACC TCCAGTCCAA |
| 401 | ATATCTTTTA TCGTATACAA ACACTTCTGG GACTGCCAGA GACTCTTGCA |
| 451 | GAAGCTGAAG AAAATCCTAC CTTCCCAAAT TCTACTATAG ATAGCCTTGC |
| 501 | AGAAATAATG ATGAACCTCG TAAGGATCTC TGATGCTGTC TCTATTTTCT |
| 551 | GGATTTTTCC TATCGTAGAT ACTACATATA ATGGAGTTTT ATTAGCCGTC |
| 601 | TGTATCGGCT TCTTCGGAAT CAATGGGATT TGTTCCACGT TCCTTATGCT |
| 651 | TACGAATCCA CGCTCTCGTC GAGATAGATG GAGGAATTTA CGCATCATGG |
| 701 | TTCTTTGCTA TCGTTCTTTG GGAAGCGGAA TGAATCTCTT TGATCTTAGC |
| 751 | AATAATGTGC GCATGGCAGC ACGTAGGCAT GTGACATCAT GTACAGTAGC |
| 801 | TCTCTATGCT ATGGTCACTC TATTTGGATG GACAGTAGCA ATACAAGATG |
| 851 | CTTTGCAATA TGGTTTCCCT AGCGTTCGGG ATGCCTTCTA TAGATATTGC |
| 901 | TTACGCCACA GATATTGCTT AACTCAAAGA AACGAAGACT CTCTGCAAAC |
| 951 | TACAGGAACG CGCTTTCAGG TTACCCGTAC ACATCTAGAA GATCAACAGA |
| 1001 | TGGTGGCTTC TATTTTGAAT TTGAGTGTTT TTGGGCTCTT TTTTGGATTC |
| 1051 | GTAGGGCTAA TGACCACGTT TGGAGGATTA GAAATCTCAC CATCTTGTCG |
| 1101 | GTGGGATGCA GCAAATAACC GAACGGTAGG TATTTTTTAG |

The PSORT algorithm predicts inner membrane (0.3102).

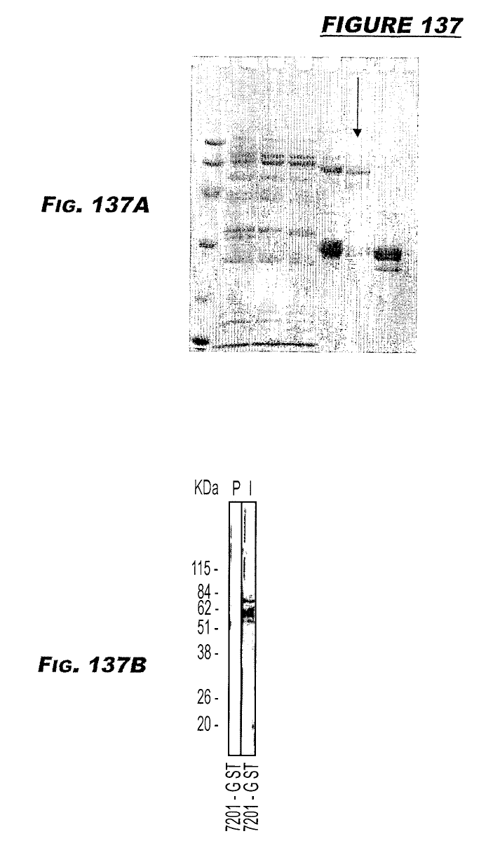

The protein was expressed in E. coli and purified as a GST-fusion product (FIG. 137A). The recombinant protein was used to immunize mice, whose sera were used in a Western blot (FIG. 137B) and for FACS analysis.

These experiments show that cp7201 is a surface-exposed and immunoaccessible protein, and that it is a useful immunogen. These properties are not evident from the sequence alone.

### Example 138

The following C. pneumoniae protein (PID 4377251) was expressed <SEQ ID 275; cp7251>:

| | |
|---|---|
| 1 | MAPIHGSNAF VEDILHSHPS PQATYFSSTR AQKLHEFKDR HPVLTRIASV |
| 51 | IIKIFKVLIG LIILPLGIYW LCQTLCTNSI LPSKNLLKIF KKQPNTKTLK |
| 101 | TNYLHALQDY SSKNRVASMR RVPILQDNVL IDTLEICLSQ APTNRWMLIS |
| 151 | LGSDCSLEEI ACKEIFDSWQ RFAKLIGANI LVYNYPGVMS STGSSSLKDL |
| 201 | ASAHNICTRY LKDKEQGPGA KEIITYGYSL GGLIQAEALR DQKIVANDDT |
| 251 | TWIAVKDRCP LFISPEGFHS CRRIGKLVAR LFGWGTKAVE RSQDLPCLEI |
| 301 | FLYPTDSLRR STVRQNKLLA PELTLAHAIK NSPYVQNKEF IEVRLSSDID |
| 351 | PIDSKTRVAL ATPILKKLS* |

The cp7251 nucleotide sequence <SEQ ID 276> is:

| | |
|---|---|
| 1 | ATGGCTCCAA TTCACGGAAG TAATGCGTTT GTTGAGGATA TTTTACATTC |
| 51 | CCACCCTTCT CCACAAGCGA CTTATTTTTC TTCAACACGC GCCCAAAAAC |
| 101 | TTCATGAGTT TAAAGACAGG CATCCCGTGC TTACACGGAT TGCTTCTGTA |
| 151 | ATTATTAAAA TTTTTAAAGT TCTGATAGGG CTGATCATCC TTCCCTTAGG |
| 201 | AATCTACTGG CTATGTCAAA CGCTTTGTAC AAACTCGATT CTCCCTTCCA |
| 251 | AGAATTTATT AAAAATTTTC AAGAAGCAAC CCAACACTAA AACCTTAAAA |
| 301 | ACTAATTATT TGCATGCTTT GCAAGATTAT TCCTCGAAAA ACCGCGTTGC |
| 351 | TTCCATGAGA CGAGTTCCTA TCCTCCAGGA TAATGTTCTC ATCGACACTT |
| 401 | TGGAAATATG CCTTTCACAA GCACCTACGA ATCGTTGGAT GCTCATTTCT |
| 451 | TTAGGAAGTG ACTGTAGCTT GGAAGAAATC GCTTGTAAGG AGATCTTTGA |
| 501 | TTCTTGGCAA AGATTTGCCA AGTTGATAGG GGCCAATATA CTCGTTTATA |
| 551 | ACTACCCCGG AGTCATGTCC AGCACAGGGA GCAGCAGCCT AAAGGACCTA |
| 601 | GCATCAGCTC ATAATATTTG TACAAGATAC CTTAAAGATA AAGAACAGGG |
| 651 | CCCTGGAGCA AAAGAAATCA TTACCTATGG GTACTCCCTA GGAGGTTTGA |
| 701 | TACAAGCAGA AGCATTGCGA GACCAGAAGA TTGTTGCAAA CGATGATACT |
| 751 | ACTTGGATAG CAGTCAAAGA TAGGTGTCCT CTCTTTATAT CTCCAGAAGG |
| 801 | TTTCCACAGT TGCAGACGCA TAGGAAAGCT AGTAGCTCGT CTTTTTGGCT |
| 851 | GGGGGACCAA AGCCGTAGAG AGAAGCCAAG ACCTTCCCTG CCTAGAAATT |
| 901 | TTTCTCTATC CTACGGATTC CTTACGAAGA TCAACAGTCA GACAGAACAA |
| 951 | GCTCTTAGCA CCTGAACTTA CTCTCGCTCA TGCGATAAAA AATAGTCCCT |
| 1001 | ATGTTCAAAA TAAAGAATTT ATAGAAGTAC GATTATCGTC TGATATCGAT |
| 1051 | CCCATCGACA GCAAAACAAG AGTGGCTCTT GCCACACCAA TTTTGAAAAA |
| 1101 | GCTCTCTTAG |

The PSORT algorithm predicts inner membrane (0.4545).

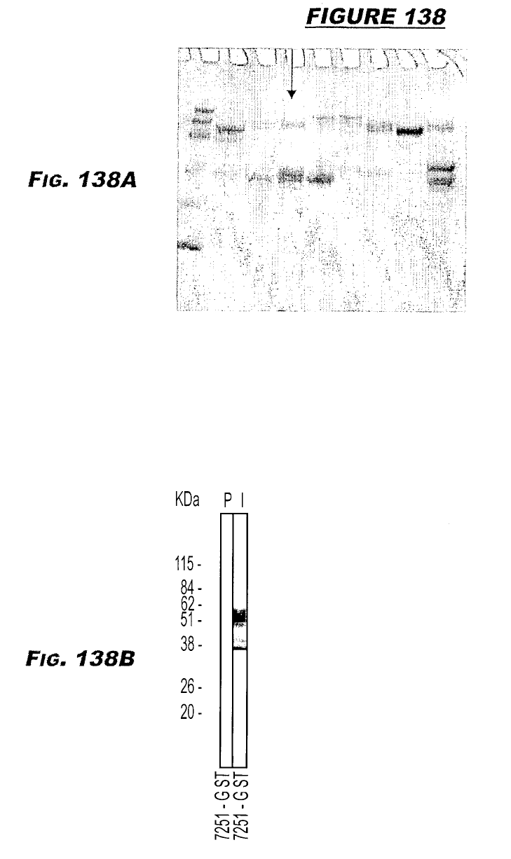

The protein was expressed in E. coli and purified as a GST-fusion product (FIG. 138A). The recombinant protein was used to immunize mice, whose sera were used in a Western blot (FIG. 138B) and for FACS analysis.

These experiments show that cp7251 is a surface-exposed and immunoaccessible protein, and that it is a useful immunogen. These properties are not evident from the sequence alone.

### Example 139

The following C. pneumoniae protein (PID 4377288) was expressed <SEQ ID 277; cp7288>:

| | |
|---|---|
| 1 | MHMSNPISLF SPAELIAKYN LIPKTSPIYP RRTELIILEE NACQTRLTNV |
| 51 | AQVLHPSSLF SMSKKILNPC GCSGGPLCWV ILNILAFIIT SVLFIILLPV |
| 101 | NLIVAGLRLF MPLPPKKIVE DLSEPTTEET NEVIQPFIFA LQALLFEDNK |
| 151 | LRSFKIVEQS VGKAPLPNPF LNRLVAISPQ ESQEAMRKIP DLCSQLKKVL |
| 201 | KSLGVLTPEW KHMLKYFEGL KNEHDSNPDK KTFPILIKLL IEALTGKSSL |
| 251 | PKTPSTKEKM QAALFIASSC KTCKPTWGEV ITRSLNRLYS IANEGDNQLL |
| 301 | IWVQEFKERE LMSIQDGDDA EEYRFAAQQH GERYTEAIEQ VLRNESAAKL |
| 351 | QWHVINTMKF FHGKNLGLVT EHLQDTLGAL TLRQTTVDTH QGREDADLSA |
| 401 | ALFLNKYLNS GNQLVNSVFK SMQKADPETK ALIREFALDI LYASLRLPQT |
| 451 | SAHTEVFSTL LMDPETYEPN KACIAYLLYV LKIIEL* |

The cp7288 nucleotide sequence <SEQ ID 278> is:

| | |
|---|---|
| 1 | ATGCATATGT CTAACCCCAT CTCTTTGTTT TCCCCTGCAG AGTTAATAGC |
| 51 | AAAGTACAAT TTAATTCCAA AAACTTCGCC GATTTATCCT CGGAGGACGG |
| 101 | AACTTATTAT CTTGGAAGAA AATGCGTGTC AAACACGCCT AACCAACGTG |
| 151 | GCTCAGGTCC TACATCCTTC TAGCCTATTC AGTATGTCAA AAAAAATACT |
| 201 | GAATCCCTGC GGGTGCTCTG GTGGTCCCTT ATGTTGGGTG ATTCTCAACA |
| 251 | TCCTAGCATT TATTATTACT TCAGTACTGT TTATCATTCT TTTACCGGTG |
| 301 | AATCTCATCG TAGCAGGTCT TCGTCTCTTC ATGCCTCTTC CCCCTAAAAA |
| 351 | AATCGTAGAG GATTTAAGTG AACCTACTAC TGAAGAAACG AATGAGGTCA |
| 401 | TTCAACCCTT CATTTTCGCT TTGCAAGCGT TGCTTTTTGA GGATAACAAA |
| 451 | CTTCGCTCTT TTAAAATTGT TGAACAAAGT GTAGGCAAAG CACCCTTACC |
| 501 | TAATCCCTTT TTAAATAGAC TAGTAGCAAT TTCGCCGCAA GAAAGCCAAG |
| 551 | AAGCCATGCG GAAGATTCCG GATCTATGCT CACAACTGAA AAAAGTATTA |
| 601 | AAGTCTCTAG GCGTGCTAAC TCCAGAATGG AAGCACATGC TGAAGTACTT |
| 651 | TGAGGGACTG AAAAACGAAC ATGATAGTAA TCCTGATAAA AAGACGTTCC |
| 701 | CAATATTGAT CAAGCTCCTC ATAGAAGCTC TTACTGGAAA GTCCTCTTTA |
| 751 | CCCAAAACTC CTAGTACAAA GGAAAAAATG CAAGCGGCCT TATTTATTGC |
| 801 | AAGTTCTTGC AAGACTTGTA AGCCGACTTG GGGAGAAGTC ATAACCAGAT |
| 851 | CTCTTAACAG ACTCTATAGT ATAGCTAATG AAGGAGACAA TCAGCTTCTG |
| 901 | ATTTGGGTTC AAGAGTTTAA AGAACGAGAG CTGATGTCCA TCCAAGATGG |
| 951 | TGATGATGCT GAAGAGTATC GGTTTGCGGC TCAGCAACAC GGTGAGCGTT |
| 1001 | ACACAGAGGC AATAGAACAA GTTCTACGAA ACGAGTCAGC AGCCAAACTA |
| 1051 | CAATGGCATG TGATCAACAC TATGAAATTC TTCCATGGGA AAAATCTCGG |
| 1101 | TCTAGTTACA GAACACCTAC AAGATACTCT CGGCGCCCTA ACTTTACGTC |
| 1151 | AAACTACAGT GGACACACAT CAAGGCAGAG AAGACGCTGA TTTGTCAGCT |
| 1201 | GCTCTTTTCC TAAATAAGTA TTTAAATTCT GGAAATCAAC TTGTTAATAG |
| 1251 | CGTCTTTAAA TCCATGCAAA AAGCAGATCC AGAAACCAAA GCTTTAATCC |
| 1301 | GTGAGTTTGC TCTAGATATA TTATATGCAT CCTTACGGCT TCCTCAAACT |
| 1351 | TCCGCTCATA CCGAGGTCTT TTCTACACTC TTAATGGACC CAGAGACCTA |
| 1401 | TGAACCTAAT AAAGCTTGTA TCGCCTACTT GCTCTATGTA TTAAAGATCA |
| 1451 | TCGAACTATA A |

The PSORT algorithm predicts inner membrane (0.5989).

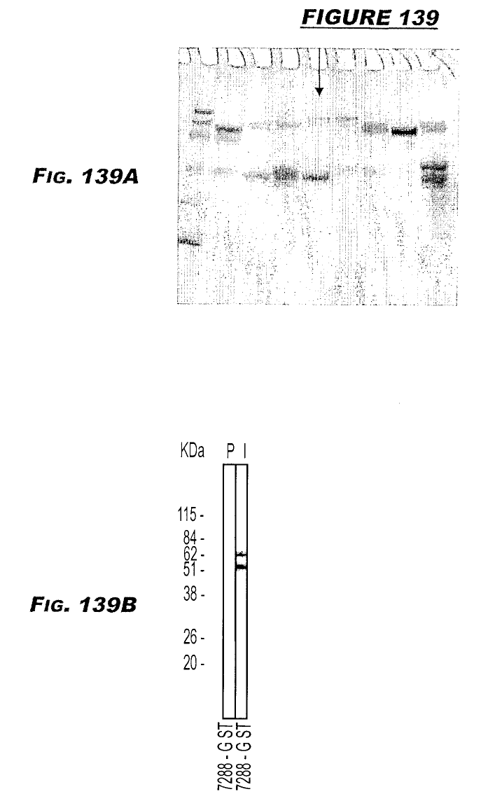

The protein was expressed in E. coli and purified as a GST-fusion product (FIG. 139A). The recombinant protein was used to immunize mice, whose sera were used in a Western blot (FIG. 139B) and for FACS analysis.

These experiments show that cp7288 is a surface-exposed and immunoaccessible protein, and that it is a useful immunogen. These properties are not evident from the sequence alone.

### Example 140

The following C. pneumoniae protein (PID 4377359) was expressed <SEQ ID 279; cp7359>:

| | |
|---|---|
| 1 | MPGSVSSPPL SPVIVRERVP SSSGSDLIQP HAVLKISILI FALVTILGIV |
| 51 | LVVLSSALGA LPSLVLTVSG CIAIAVGLIG LGILVTRLIL STIRKVDAMG |
| 101 | YDAAVKEEQY LSRIRELESE NEEIRDRNRA VEDQCAHLSE ENKDLRDPEY |
| 151 | LHGMTERLIA SLEIENQALV AENILLKDWN ASLSRDFRAY KQKFPLGALE |
| 201 | PWKEDIACIM EQNLFLKPEC IAMVKSLPLE TQRLFLYPKG FQSLVNRFAP |
| 251 | RSRFFQTPKY EYNSRNENED GKVAAVCARL KKEFFSAVLG ACSYEELGGI |
| 301 | CERAVALKET LPLPEAVYDT LVQEFPNLLT AESLWKEWCF YSYPYLRPYL |
| 351 | SVDYCKRLFV QLFEELCLKL FTTGSPEDQA LVRLFSYYRN HIPAVLASFG |
| 401 | LPPPETGGSV FVLLPKQENL LWSQIEVLAT RYLKDTFVRN SEWTGSFEMM |
| 451 | FSYNEMCKEI SEGRIRFAED YETRHSEEFP PSPLSEEGEG EEFLPPCSEE |
| 501 | EVSVLERPDL DVDSMWVWHP PVPKGPL* |

The cp7359 nucleotide sequence <SEQ ID 280> is:

| | |
|---|---|
| 1 | ATGCCAGGTT CTGTGTCATC ACCTCCTTTG TCTCCTGTAA TTGTCCGTGA |
| 51 | AAGGGTCCCA TCCTCTTCAG GATCCGACCT CATACAGCCT CATGCTGTTT |
| 101 | TAAAGATCTC CATCCTAATT TTTGCGCTTG TGACAATTTT AGGAATTGTT |
| 151 | CTTGTAGTGT TGTCTAGTGC TTTAGGAGCT CTTCCTAGTT TAGTTTTGAC |
| 201 | GGTTTCTGGT TGTATTGCAA TAGCTGTAGG CCTGATTGGT TTAGGGATTC |
| 251 | TTGTGACACG GCTGATTCTC TCTACGATCA GAAAAGTAGA TGCCATGGGT |
| 301 | TATGATGCTG CGGTCAAAGA AGAGCAGTAT TTGTCACGTA TCAGAGAATT |
| 351 | AGAGTCTGAA AATAGAGAGA TTAGAGATAG AAATCGTGCT GTCGAAGATC |
| 401 | AGTGTGCCCA TTTATCCGAA GAGAACAAGG ACCTTAGGGA TCCCGAATAT |
| 451 | CTACATGGAA TGACTGAAAG GCTCATTGCG AGCTTAGAAA TAGAGAATCA |
| 501 | AGCTCTCGTA GCTGAGAACA TTCTTCTCAA AGACTGGAAT GCAAGCCTAT |
| 551 | CTAGAGATTT CCGCGCATAT AAGCAAAAAT TTCCTCTTGG GGCATTAGAA |
| 601 | CCCTGGAAAG AAGATATTGC ATGTATCATG GAACAAAATC TCTTTTTAAA |
| 651 | ACCGGAATGT ATCGCGATGG TTAAGTCTCT TCCATTAGAG ACGCAACGGC |
| 701 | TGTTTTTATA TCCAAAAGGA TTTCAGTCTT TAGTTAATCG ATTTGCTCCG |
| 751 | CGGTCTCGCT TTTTCCAGAC TCCAAAGTAT GAATATAACA GTAGGAATGA |
| 801 | AAATGAGGAC GGAAAGGTAG CCGCAGTGTG CGCCCGTTTG AAAAAAGAAT |
| 851 | TCTTCAGTGC TGTTTTAGGA GCCTGTAGTT ACGAAGAACT AGGGGGCATT |
| 901 | TGTGAAAGAG CAGTAGCACT TAAAGAGACG TTGCCATTGC CTGAAGCTGT |
| 951 | CTATGATACC CTAGTTCAGG AGTTCCCAAA TCTTCTTACT GCTGAGAGTT |
| 1001 | TATGGAAAGA ATGGTGCTTC TATTCCTATC CCTACCTTCG TCCCTATCTT |
| 1051 | TCTGTGGATT ACTGTAAGAG GTTATTTGTA CAACTTTTTG AGGAACTCTG |
| 1101 | CCTAAAGCTT TTTACAACGG GATCTCCAGA AGACCAAGCT TTGGTTCGCC |
| 1151 | TTTTCTCTTA CTATAGGAAT CATATTCCCG CAGTCTTGGC CTCATTTGGT |
| 1201 | TTGCCCCCGC CTGAGACAGG GGGGTCTGTA TTTGTATTGC TACCAAAACA |
| 1251 | AGAAAACCTT CTTTGGAGTC AAATTGAGGT GCTGGCTACA AGGTATCTCA |
| 1301 | AAGATACCTT CGTGAGAAAC TCAGAATGGA CGGGCTCTTT CGAGATGATG |
| 1351 | TTTTCTTATA ACGAGATGTG TAAGGAGATC TCCGAAGGAA GGATTCGTTT |
| 1401 | TGCTGAAGAC TATGAAACGA GGCATTCCGA AGAATTCCCT CCTTCCCCTC |
| 1451 | TCTCTGAAGA AGGAGAGGGC GAAGAATTCC TTCCTCCTTG CTCTGAAGAA |
| 1501 | GAGGTTTCGG TTCTTGAGCG CCCAGATCTA GATGTAGACT CTATGTGGGT |
| 1551 | CTGGCATCCG CCGGTCCCTA AGGGACCTCT TTAA |

The PSORT algorithm predicts inner membrane (0.7453).

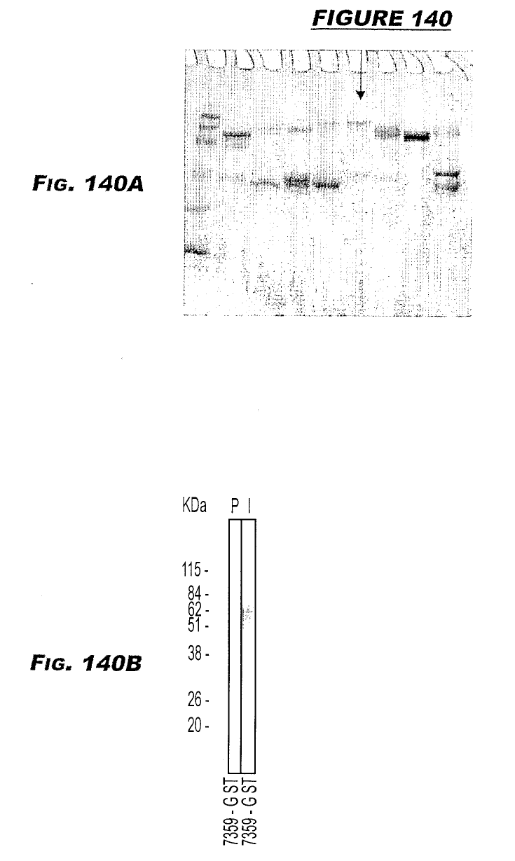

The protein was expressed in E. coli and purified as a GST-fusion product (FIG. 140A). The recombinant protein was used to immunize mice, whose sera were used in a Western blot (FIG. 140B) and for FACS analysis.

These experiments show that cp7359 is a surface-exposed and immunoaccessible protein, and that it is a useful immunogen. These properties are not evident from the sequence alone.

### Example 141

The following C. pneumoniae protein (PID 4377374) was expressed <SEQ ID 281; cp7374>:

| | |
|---|---|
| 1 | MDKQSSGNSG CIWHPFTQSA LDSTPIKIVR GEGAYLYAES GTRYLDAISS |
| 51 | WWCNLHGHGH PYITKKLCEQ AQKLEHVIFA NFTHEPALEL VSKLAPLLPE |
| 101 | GLERFFFSDN GSTSIEIAMK IAVQYYYNQN KAKSHFVGLS NAYHGDTFGA |
| 151 | MSIAGTSPTT VPFHDLFLPS STIAAPYYGK EELAIAQAKT VFSESNIAAF |
| 201 | IYEPLLQGAG GMLMYNPEGL KEILKLAKHY GVLCIADEIL TGFGRTGPLF |
| 251 | ASEFTDIPPD IICLSKGLTG GYLPLALTVT TKEIHDAFVS QDRMKALLHG |
| 301 | HTFTGNPLGC SAALASLDLT LSPECLQQRQ MIERCHQEFQ EAHGSLWQRC |
| 351 | EVLGTVLALD YPAEATGYFS QYRDHLNRFF LERGVLLRPL GNTLYVLPPY |
| 401 | CIQEEDLRII YSHLQDALCL QPQ* |

The cp7374 nucleotide sequence <SEQ ID 282> is:

| | |
|---|---|
| 1 | ATGGACAAGC AATCATCAGG GAATTCAGGG TGTATCTGGC ACCCCTTCAC |
| 51 | TCAATCTGCA TTAGATTCTA CACCCATAAA GATTGTAAGG GGAGAAGGTG |
| 101 | CTTACCTCTA TGCGGAATCA GGAACAAGAT ATCTTGATGC GATATCTTCA |
| 151 | TGGTGGTGCA ACCTCCACGG TCATGGGCAT CCCTACATTA CAAAAAAATT |
| 201 | ATGTGAGCAA GCACAGAAGT TAGAACATGT GATCTTCGCA AATTTCACCC |
| 251 | ATGAACCGGC TCTAGAGCTC GTATCGAAAC TCGCTCCCCT CCTTCCTGAA |
| 301 | GGTCTAGAAC GTTTCTTTTT CTCTGACAAC GGATCAACGT CTATCGAAAT |
| 351 | AGCAATGAAA ATTGCTGTGC AATATTACTA CAATCAAAAC AAGGCTAAGA |
| 401 | GCCATTTTGT TGGACTCAGC AATGCCTATC ACGGAGATAC ATTTGGAGCT |
| 451 | ATGTCGATAG CTGGCACGAG CCCTACTACA GTTCCCTTTC ATGATCTTTT |
| 501 | TCTTCCTTCC AGTACAATTG CTGCTCCCTA TTATGGCAAG GAAGAGCTTG |
| 551 | CCATTGCCCA AGCAAAAACA GTCTTTTCTG AAAGCAATAT CGCAGCGTTT |
| 601 | ATCTATGAGC CGCTATTGCA AGGTGCTGGA GGGATGTTAA TGTATAATCC |
| 651 | CGAAGGCCTA AAGGAGATTC TCAAGCTTGC CAAGCATTAC GGGGTTCTCT |
| 701 | GTATTGCTGA TGAAATTCTT ACTGGCTTTG GCCGTACGGG TCCACTGTTT |
| 751 | GCTTCTGAAT TTACAGACAT TCCTCCTGAC ATTATCTGTC TTTCTAAAGG |
| 801 | TCTTACAGGA GGCTATCTCC CTCTAGCCTT GACAGTAACC ACTAAAGAAA |
| 851 | TTCATGATGC CTTTGTCTCC CAAGATCGGA TGAAGGCACT GCTTCATGGC |
| 901 | CATACCTTCA CAGGAAATCC TTTAGGCTGT AGTGCTGCCC TCGCTTCTTT |
| 951 | GGATCTCACC CTATCTCCAG AATGCCTACA ACAAAGGCAA ATGATAGAAC |
| 1001 | GGTGTCATCA AGAGTTTCAA GAAGCTCATG GTTCCCTATG GCAACGGTGT |
| 1051 | GAGGTTCTGG GCACGGTACT CGCTCTAGAT TACCCTGCAG AAGCTACAGG |
| 1101 | ATATTTTTCA CAATATAGAG ACCATCTCAA TCGCTTTTTC TTAGAACGTG |
| 1151 | GAGTCCTTCT TCGTCCTTTA GGGAACACAC TGTATGTGCT GCCCCCCTAC |
| 1201 | TGTATCCAAG AAGAAGATCT CCGGATTATT TATTCTCACC TACAGGATGC |
| 1251 | CCTATGTCTA CAACCACAGT AA |

The PSORT algorithm predicts cytoplasm (0.2930).

The protein was expressed in E. coli and purified as a GST-fusion product (FIG. 141A) and also in his-tagged form. The recombinant proteins were used to immunize mice, whose sera were used in a Western blot (FIG. 141B) and for FACS analysis.

These experiments show that cp7374 is a surface-exposed and immunoaccessible protein, and that it is a useful immunogen. These properties are not evident from the sequence alone.

### Example 142

The following C. pneumoniae protein (PID 4377377) was expressed <SEQ ID 283; cp7377>:

| | |
|---|---|
| 1 | MREETVSWSL EDIREIYHTP VFELIHKANA ILRSNFLHSE LQTCYLISIK |
| 51 | TGGCVEDCAY CAQSSRYHTH VTPEPMMKIV DVVERAKRAV ELGATRVCLG |
| 101 | AAWRNAKDDR YFDRVLAMVK SITDLGAEVC CALGMLSEEQ AKKLYDAGLY |
| 151 | AYNHNLDSSP EFYETIITTR SYEDRLNTLD VVNKSGISTC CGGIVGMGES |
| 201 | EEDRIKLLHV LATRDHIPES VPVNLLWPID GTPLQDQPPI SFWEVLRTIA |
| 251 | TARVVFPRSM VRLAAGRAFL TVEQQTLCFL AGANSIFYGD KLLTVENNDI |
| 301 | DEDAEMIKLL GLIPRPSFGI ERGNPCYANN S* |

The cp7377 nucleotide sequence <SEQ ID 284> is:

| | |
|---|---|
| 1 | ATGCGTGAAG AAACTGTATC CTGGTCATTA GAAGACATCC GCGAAATTTA |
| 51 | TCACACTCCC GTATTTGAGC TGATTCACAA AGCCAATGCC ATATTGCGTA |
| 101 | GTAATTTCCT CCATTCAGAA CTGCAGACTT GCTATCTGAT TTCGATTAAA |
| 151 | ACTGGTGGAT GCGTTGAAGA TTGCGCCTAC TGTGCCCAAT CTTCCCGCTA |
| 201 | TCATACCCAC GTCACACCAG AACCTATGAT GAAAATTGTA GACGTTGTGG |
| 251 | AAAGGGCAAA ACGTGCTGTA GAGCTAGGCG CCACTCGTGT GTGTCTTGGG |
| 301 | GCTGCCTGGC GCAATGCTAA GGACGATCGA TACTTTGATA GAGTCCTCGC |
| 351 | TATGGTGAAA AGTATCACAG ATCTCGGAGC CGAGGTTTGT TGTGCTTTAG |
| 401 | GCATGCTCTC CGAAGAGCAA GCTAAAAAAC TGTATGATGC AGGACTTTAT |
| 451 | GCCTACAATC ATAATTTAGA CTCTTCTCCG GAATTCTATG AAACTATAAT |
| 501 | CACAACACGT TCTTATGAAG ATCGCCTCAA CACTCTTGAT GTAGTAAATA |
| 551 | AATCTGGCAT TAGTACATGC TGCGGTGGTA TTGTAGGTAT GGGAGAATCT |
| 601 | GAAGAAGACC GTATAAAGCT TCTTCATGTT CTTGCAACAA GAGATCATAT |
| 651 | CCCAGAATCC GTACCTGTAA ATTTACTTTG GCCGATTGAC GGCACGCCTT |
| 701 | TGCAAGACCA GCCTCCGATT TCTTTCTGGG AAGTCTTGCG AACCATAGCA |
| 751 | ACGGCACGGG TTGTTTTCCC CAGATCCATG GTACGACTTG CTGCAGGACG |
| 801 | CGCTTTCCTC ACAGTAGAAC AACAAACCTT ATGTTTTCTA GCCGGTGCCA |
| 851 | ACTCCATATT CTATGGAGAT AAACTGTTGA CTGTAGAAAA CAATGATATA |
| 901 | GATGAAGATG CTGAAATGAT CAAACTTTTA GGCTTAATCC CTCGCCCTTC |
| 951 | ATTTGGAATA GAAAGAGGTA ACCCATGTTA TGCCAACAAT TCCTAA |

The PSORT algorithm predicts cytoplasm (0.2926).

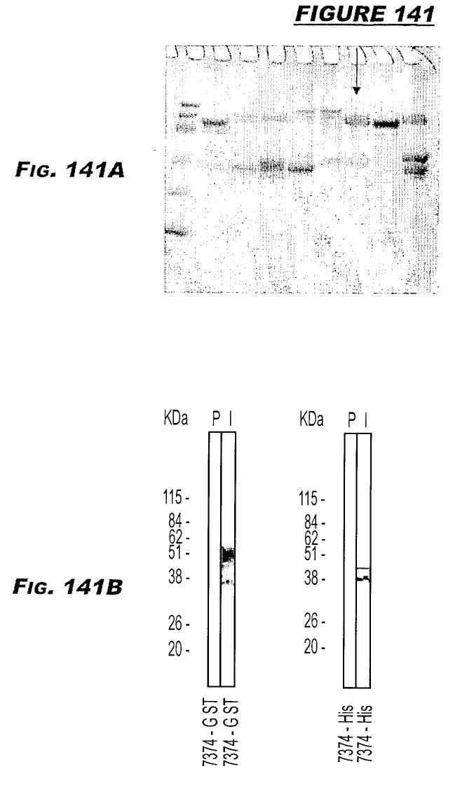

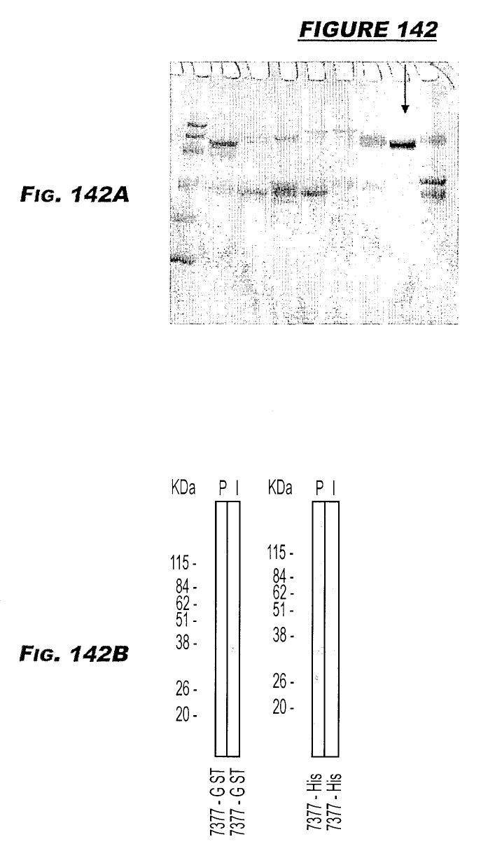

The protein was expressed in E. coli and purified as a GST-fusion product (FIG. 142A) and also in his-tagged form. The recombinant proteins were used to immunize mice, whose sera were used in a Western blot (FIG. 142B) and for FACS analysis.

These experiments show that cp7377 is a surface-exposed and immunoaccessible protein, and that it is a useful immunogen. These properties are not evident from the sequence alone.

### Example 143

The following C. pneumoniae protein (PID 4377407) was expressed <SEQ ID 285; cp7407>:

| | |
|---|---|
| 1 | MVCPNNSWFR MCGNFNCEWV EVTTTEETTR QSASDISEEA GSSGGAAPIT |
| 51 | TQPTKITKVE KRVQFNTAQG DESTIHMIQE AGELVDSILS HRRTQGCTEY |
| 101 | CYDSYATGCG QRCGSFGRLI CGTYKACCLD REDNQVAGLV HECEQTHGPI |
| 151 | AVALAAKTMG LNLMELVEKN TILSEEQKNE FRQHCSEAKT QLYGTMQSLS |
| 201 | QNFFLEGVNS IRERGLDDSL VQAVLSFIAT RSWEKTIESE EASGTSSASN |
| 251 | STRIPACYIL NTSPLTTSRL SCGSRDARRP SSVGAEPQYV AKKYNDNGMA |
| 301 | RQLGKIQVTN LKTGDFSALG PFGLLIVKML NSFLLSASQS TSSILKHTGG |
| 351 | EICYTCPNFR DIVVLLMLAI GYCPANTDET SVVDIHMIDD PIMTIFYRLQ |
| 401 | YSYRTGKTSA SFLKKKPSLV RQESLDCPTP AESVPLMSSL EEEDENEDDD |
| 451 | EDGNLAYQQR ILECSGHLQT LELGIKINKE * |

The cp7407 nucleotide sequence <SEQ ID 286> is:

| | |
|---|---|
| 1 | ATGGTTTGCC CAAATAATTC TTGGTTCAGA ATGTGTGGAA ATTTCAACTG |
| 51 | CGAATGGGTT GAAGTAACAA CAACAGAAGA AACAACGCGG CAATCGGCTT |
| 101 | CAGATATAAG CGAAGAAGCT GGTTCGAGTG GAGGAGCTGC TCCTATAACT |
| 151 | ACGCAACCTA CTAAAATTAC AAAAGTAGAG AAACGTGTCC AATTTAATAC |
| 201 | TGCTCAAGGT GATGAAAGTA CAATACACAT GATCCAAGAA GCAGGAGAAT |
| 251 | TGGTAGACTC CATTCTATCA CATAGACGAA CGCAAGGATG TACAGAGTAT |
| 301 | TGTTATGACA GTTACGCAAC TGGATGTGGT CAGCGTTGCG GATCTTTTGG |
| 351 | AAGACTCATT TGTGGAACGT ATAAAGCGTG TTGCTTAGAC AGAGAGGATA |
| 401 | ATCAGGTTGC TGGACTTGTC CATGAATGCG AACAGACCCA TGGTCCTATT |
| 451 | GCCGTTGCTT TAGCTGCTAA AACTATGGGC CTCAACTTAA TGGAACTTGT |
| 501 | AGAAAAAAAC ACTATTTTGT CTGAAGAACA GAAAAATGAA TTTAGACAGC |
| 551 | ATTGCTCGGA AGCTAAAACC CAACTCTATG GAACGATGCA GAGCCTTTCT |
| 601 | CAAAACTTTT TCCTTGAAGG AGTCAACAGC ATTAGAGAAC GCGGTCTAGA |
| 651 | CGATTCACTA GTCCAAGCCG TGCTAAGCTT TATTGCTACA AGGTCTTGGG |
| 701 | AAAAAACTAT AGAATCAGAG GAAGCCTCAG GAACATCTTC TGCTTCTAAT |
| 751 | TCTACACGCA TTCCTGCGTG CTATATCTTA AATACGAGCC CCTTAACGAC |
| 801 | GTCACGCCTA TCCTGTGGAT CAAGAGATGC GCGACGCCCA TCTTCAGTCG |
| 851 | GTGCAGAGCC CCAGTACGTA GCAAAAAAAT ACAATGACAA TGGCATGGCC |
| 901 | AGACAATTAG GAAAAATCCA AGTCACCAAT CTAAAAACAG GAGATTTTTC |
| 951 | AGCTTTAGGT CCTTTTGGTC TCCTGATTGT GAAAATGCTG AATAGCTTTC |
| 1001 | TCTTATCTGC ATCACAAAGC ACATCTTCTA TTCTAAAGCA CACAGGTGGA |
| 1051 | GAAATATGTT ATACGTGCCC AAATTTTCGT GATATCGTCG TTTTATTGAT |
| 1101 | GTTAGCGATT GGCTATTGCC CTGCAAATAC CGATGAGACA TCTGTCGTAG |
| 1151 | ATATACACAT GATAGATGAT CCGATTATGA CCATCTTCTA TCGACTACAA |
| 1201 | TACAGCTATA GAACAGGGAA AACTTCAGCA TCGTTTTTAA AAAAGAAACC |
| 1251 | CTCATTAGTA AGACAGGAAA GTCTTGATTG TCCTACCCCT GCAGAATCTG |
| 1301 | TCCCTCTCAT GTCAAGTCTC GAAGAAGAAG ATGAAAATGA AGATGATGAT |
| 1351 | GAGGATGGGA ATTTGGCGTA TCAACAGCGT ATCCTTGAAT GCTCGGGTCA |
| 1401 | TTTACAAACT CTATTTTTAG GGATAAAAAT AAACAAAGAA TAA |

The PSORT algorithm predicts inner membrane (0.1319).

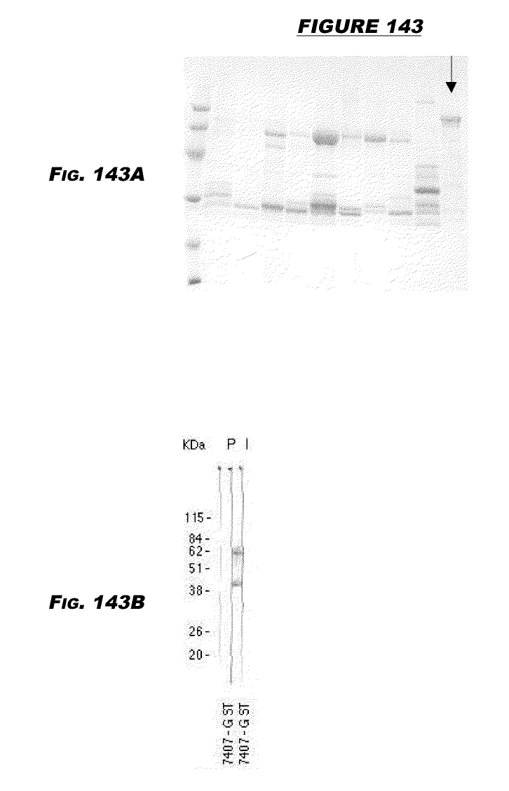

The protein was expressed in E. coli and purified as a GST-fusion product (FIG. 143A). The recombinant protein was used to immunize mice, whose sera were used in a Western blot (FIG. 143B) and for FACS analysis.

These experiments show that cp7407 is a surface-exposed and immunoaccessible protein, and that it is a useful immunogen. These properties are not evident from the sequence alone.

### Example 144

The following C. pneumoniae protein (PID 4376432) was expressed <SEQ ID 287; cp6432>:

| | |
|---|---|
| 1 | MTRSTIESSD SLCSRSFSQK LSVQTLKNLC ESRLMKITSL VIAFLTLIVG |
| 51 | GALIALAGGG VLSFPLGLIL GSVLVLFSSI YLVSCCKFFT LKEMTMTCSV |
| 101 | KSKINIWFEK QRNKDIEKAL ENPDLFGENK RNVGNRSARN QLEMILHETD |
| 151 | GIILKRYMKG AKMYFYL* |

The cp6432 nucleotide sequence <SEQ ID 288> is:

| | |
|---|---|
| 1 | ATGACTAGAA GTACTATTGA AAGCAGTGAT TCGCTATGCT CAAGGTCTTT |
| 51 | TTCTCAAAAA TTAAGTGTCC AGACATTAAA AAATCTCTGT GAAAGTAGAT |
| 101 | TAATGAAGAT CACTTCTCTT GTGATTGCTT TCCTAACTCT AATTGTGGGG |
| 151 | GGTGCTCTTA TAGCTTTAGC AGGAGGGGGG GTTCTTTCTT TCCCTCTTGG |
| 201 | GCTAATCTTA GGAAGCGTAC TCGTTTTGTT TTCTTCTATC TATTTAGTCT |
| 251 | CTTGTTGTAA ATTTTTTACT TTAAAAGAGA TGACAATGAC CTGTAGTGTC |
| 301 | AAATCTAAAA TCAATATATG GTTTGAAAAG CAACGAAACA AAGACATCGA |
| 351 | AAAGGCATTA GAGAATCCAG ATCTCTTTGG AGAAAATAAG AGAAATGTTG |
| 401 | GAAATCGTTC GGCAAGAAAT CAACTAGAAA TGATCTTACA CGAGACTGAC |
| 451 | GGAATTATTT TGAAAAGATA TATGAAAGGA GCTAAAATGT ACTTTTATTT |
| 501 | ATGA |

The PSORT algorithm predicts inner membrane (0.5394).

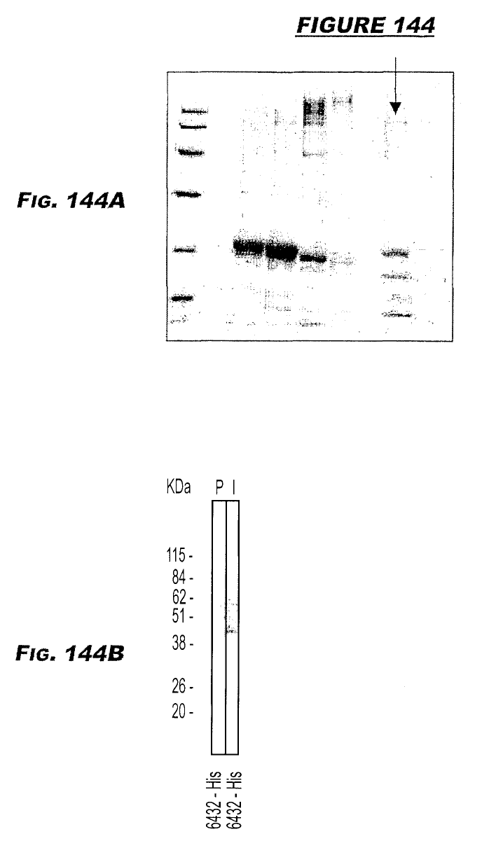

The protein was expressed in E. coli and purified as a his-tagged product (FIG. 144A). The recombinant protein was used to immunize mice, whose sera were used in a Western blot (FIG. 144B) and for FACS analysis.

These experiments show that cp6432 is a surface-exposed and immunoaccessible protein, and that it is a useful immunogen. These properties are not evident from the sequence alone.

### Example 145

The following C. pneumoniae protein (PID 4376433) was expressed <SEQ ID 289; cp6433>:

| | |
|---|---|
| 1 | MNWVPKTIDH VDPESEIDIR KVVSCYKLIK ECQPEFRSLI SELLGVIRCG |
| 51 | LRLLKRSKYQ EQARTVSDED APLFCLTRSY YQDGYLTPLR AGPRDLINHY |
| 101 | IHLRRRENPK HFFSPKHPCY YARLAFNESV CVYRELFDIE RLTKMYVEGD |
| 151 | YSKEQEKNLQ AILSFVKTLD EGKDFLIEHK DTDLIGRGFT DVFCT* |

The cp6433 nucleotide sequence <SEQ ID 290> is:

| | |
|---|---|
| 1 | ATGAATTGGG TTCCAAAAAC AATAGACCAT GTAGATCCAG AATCAGAGAT |
| 51 | AGATATACGT AAAGTCGTCT CCTGCTATAA GTTGATAAAA GAATGTCAAC |
| 101 | CTGAATTTCG ATCTCTTATA AGTGAATTAC TAGGAGTGAT TCGGTGTGGC |
| 151 | TTAAGACTAT TAAAACGTTC TAAGTATCAA GAACAGGCTA GAACTGTATC |
| 201 | TGATGAAGAT GCACCTCTTT TCTGCCTGAC TCGTTCTTAT TATCAAGATG |
| 251 | GTTATCTCAC GCCATTAAGA GCAGGACCTC GTGATCTTAT AAATCACTAT |
| 301 | ATACACTTGC GTCGCCGAGA GAATCCTAAG CATTTTTTCA GTCCTAAGCA |
| 351 | TCCATGTTAT TATGCTCGAT TGGCTTTTAA TGAGTCAGTG TGTGTCTATA |
| 401 | GAGAACTCTT TGATATAGAG CGACTTACAA AAATGTATGT CGAGGGTGAT |
| 451 | TATTCTAAAG AACAAGAGAA AAACCTACAG GCTATTCTTA GTTTTGTGAA |
| 501 | AACTCTAGAT GAAGGAAAGG ACTTTCTTAT TGAACATAAA GATACCGATC |
| 551 | TCATTGGGAG AGGTTTTACT GATGTGTTCT GCACTTAA |

The PSORT algorithm predicts cytoplasm (0.4068).

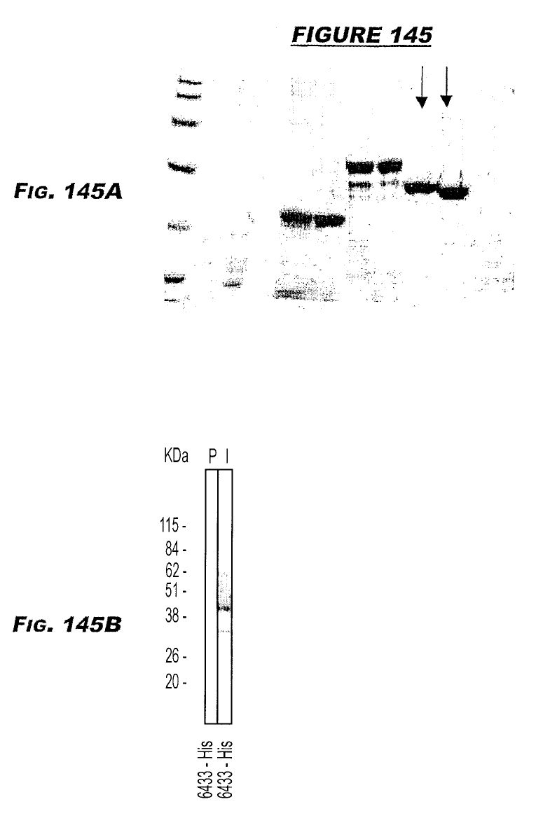

The protein was expressed in E. coli and purified as a his-tagged product (FIG. 145A). The recombinant protein was used to immunize mice, whose sera were used in a Western blot (FIG. 145B) and for FACS analysis.

These experiments show that cp6433 is a surface-exposed and immunoaccessible protein, and that it is a useful immunogen. These properties are not evident from the sequence alone.

### Example 146

The following C. pneumoniae protein (PID 4376643) was expressed <SEQ ID 291; cp6643>:

| | |
|---|---|
| 1 | MGYLPVSATD VLFESPAAPL INSANTQNQK LIELKGKQQA ESSPRTITSV |
| 51 | ILEVLLVIGC CLIVLSLLAI RPALQFTLET GHPAAIAVLA VSGTILLVAV |
| 101 | IILFCFLAAV PFAAKKTYKY VKTVDDYASW HSHQQTPTLG TIFSGIVYAE |
| 151 | SQAQL* |

The cp6643 nucleotide sequence <SEQ ID 292> is:

| | |
|---|---|
| 1 | ATGGGATATC TTCCAGTATC TGCTACGGAC GTTCTTTTTG AAAGTCCAGC |
| 51 | CGCTCCCTTA ATCAATAGCG CAAACACACA AAATCAGAAA CTCATAGAAC |
| 101 | TCAAGGGGAA GCAGCAAGCT GAGTCTTCTC CACGGACAAT CACTTCTGTC |
| 151 | ATATTGGAAG TTCTCCTAGT GATCGGATGC TGCCTCATAG TTCTTAGTTT |
| 201 | ATTGGCAATC CGCCCTGCTC TGCAATTCAC TCTAGAAACT GGACATCCAG |
| 251 | CTGCCATTGC AGTCCTTGCT GTCTCAGGAA CAATTCTATT GGTGGCTGTT |
| 301 | ATCATCTTGT TTTGCTTTCT AGCAGCTGTG CCATTCGCTG CTAAGAAAAC |
| 351 | TTATAAATAT GTTAAGACGG TTGATGACTA TGCTTCTTGG CATTCTCATC |
| 401 | AGCAAACACC GACCCTAGGC ACTATCTTTT CAGGTATCGT CTATGCAGAA |
| 451 | TCCCAGGCGC AATTATAG |

The PSORT algorithm predicts inner membrane (0.6859).

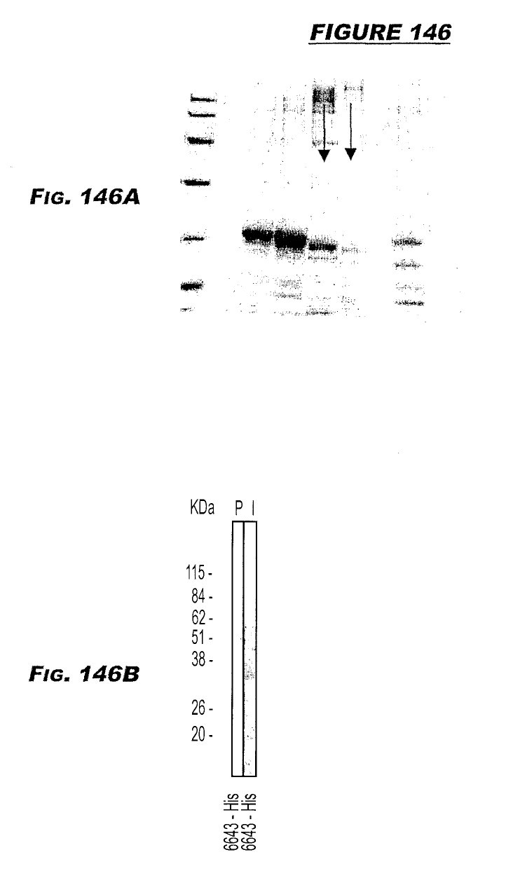

The protein was expressed in E. coli and purified as a his-tagged product (FIG. 146A). The recombinant protein was used to immunize mice, whose sera were used in a Western blot (FIG. 146B) and for FACS analysis.

These experiments show that cp6643 is a surface-exposed and immunoaccessible protein, and that it is a useful immunogen. These properties are not evident from the sequence alone.

### Example 147

The following C. pneumoniae protein (PID 4376722) was expressed <SEQ ID 293; cp6722>:

| | |
|---|---|
| 1 | VSSTLNGVFP SSLPEESADL FITNKEIVAL GEKGNVFLTH SIPMHIAAIT |
| 51 | ILVIVALAGI AIICLGCYSQ SILLIAVGIV LTILTLLCLQ ALVGFIKFIR |
| 101 | QLPQQLHTTV QFIREKIRPE SSLQLVTNAQ RKTTQDTLKL YEELCDLSQK |
| 151 | EFKLQSTLYQ KRFELSHKNE KTNQN* |

The cp6722 nucleotide sequence <SEQ ID 294> is:

| | |
|---|---|
| 1 | GTGTCTAGTA CTTTAAACGG GGTATTTCCC TCATCCCTTC CGGAAGAGTC |
| 51 | TGCTGATTTA TTCATTACGA ATAAGGAGAT CGTAGCTTTG GGGGAGAAGG |
| 101 | GCAATGTTTT TCTCACCCAC TCCATTCCTA TGCATATTGC TGCGATTACG |
| 151 | ATCTTAGTGA TTGTAGCTCT TGCTGGAATC GCTATTATCT GTTTGGGTTG |
| 201 | CTATAGCCAA AGCATTCTGT TGATTGCCGT TGGCATTGTT CTTACTATTT |
| 251 | TGACTCTTCT CTGCCTACAA GCCTTGGTAG GATTTATTAA ATTCATCCGG |
| 301 | CAGCTCCCTC AGCAGCTCCA TACGACAGTA CAATTTATCA GGGAGAAGAT |
| 351 | TCGACCTGAA TCCTCTCTAC AGCTTGTAAC CAATGCACAG AGAAPAACCA |
| 401 | CTCAAGATAC GCTAAAGTTA TACGAAGAAC TCTGCGACCT CTCACAAAAA |
| 451 | GAGTTCAAAC TGCAATCAAC TCTTTATCAA AAACGTTTTG AGCTTTCTCA |
| 501 | CAAGAATGAA AAGACAAATC AAAACTAG |

The PSORT algorithm predicts inner membrane (0.6668).

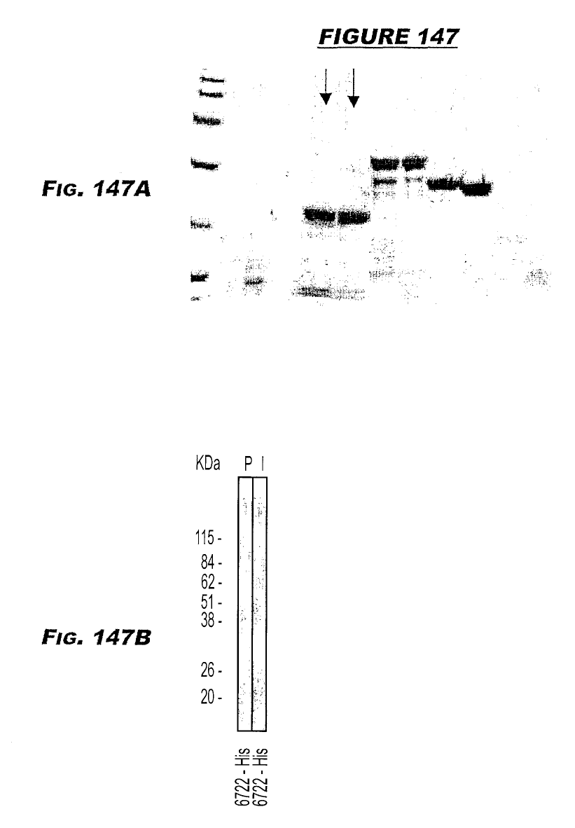

The protein was expressed in E. coli and purified as a his-tagged product (FIG. 147A). The recombinant protein was used to immunize mice, whose sera were used in a Western blot (FIG. 147B) and for FACS analysis.

These experiments show that cp6722 is a surface-exposed and immunoaccessible protein, and that it is a useful immunogen. These properties are not evident from the sequence alone.

### Example 148

The following C. pneumoniae protein (PID 4377253) was expressed <SEQ ID 295; cp7253>:

| | |
|---|---|
| 1 | MSELAPCSTG LQMVPHTQVH HALDTRRVIL TIAACLSLIA GIVLVGLGAA |
| 51 | AILPSLFGVI GGMILILFSS IALIYLYKKT REVDQIALEP LPEMISKDQS |
| 101 | IIDFVKTRDY ASLEKKATFA YTHTHYYDGS MVFYREIPRF MLGSYLALRK |
| 151 | DMDRQALF* |

The cp7253 nucleotide sequence <SEQ ID 296> is:

| | |
|---|---|
| 1 | ATGAGCGAGC TCGCCCCCTG CTCGACAGGA TTGCAGATGG TCCCCCATAC |
| 51 | GCAGGTCCAT CATGCCCTTG ATACGCGGAG AGTCATTCTA ACGATAGCCG |
| 101 | CCTGTCTGTC TTTAATTGCA GGAATCGTGT TGGTTGGCTT AGGTGCTGCA |
| 151 | GCAATCCTGC CCTCGCTTTT TGGAGTCATT GGAGGAATGA TTCTTATTCT |
| 201 | GTTTTCTTCG ATCGCCCTCA TTTATTTATA CAAGAAGACA AGGGAGGTGG |
| 251 | ATCAGATTGC TCTGGAGCCT CTTCCTGAGA TGATTTCTAA AGATCAAAGC |
| 301 | ATTATAGATT TTGTAAAGAC ACGAGACTAT GCATCTTTAG AAAAGAAAGC |
| 351 | GACCTTTGCT TATACTCATA CTCATTATTA CGATGGAAGC ATGGTCTTCT |
| 401 | ATAGGGAGAT CCCTAGATTT ATGTTAGGCT CTTATCTCGC GCTTCGCAAA |
| 451 | GACATGGACC GCCAAGCTCT TTTTTGA |

The PSORT algorithm predicts inner membrane (0.5394).

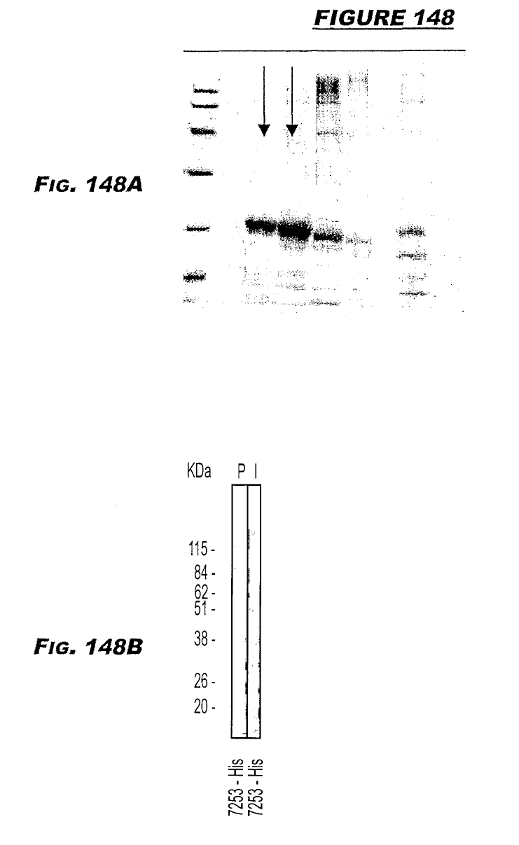

The protein was expressed in E. coli and purified as a his-tagged product (FIG. 148A). The recombinant protein was used to immunize mice, whose sera were used in a Western blot (FIG. 148B) and for FACS analysis.

These experiments show that cp7253 is a surface-exposed and immunoaccessible protein, and that it is a useful immunogen. These properties are not evident from the sequence alone.

### Example 149

The following C. pneumoniae protein (PID 4376264) was expressed <SEQ ID 297; cp6264>:

| | |
|---|---|
| 1 | VISGLLFLLV RREVPTVRSE EIPRGVSVTP SEEPALEKAQ KEPETKKILD |
| 51 | RLPKELDQLD TYIQEVFACL ERLKDPKYED RGLLTEAKEK LRVFDVVEKD |
| 101 | MMSEFLDIQR VLNEEAYYVE HCQDPLENIA YEIFSSQELR DYYCAGVCGY |
| 151 | LPSGDARADR LKRSVKEVMD RFMRVTWKSW EASVMLDHSY GVARELFKKA |
| 201 | VGVLEESVYK ILEKSYRDAF YECEKAKIQR DGRFKWL* |

The cp6264 nucleotide sequence <SEQ ID 298> is:

| | |
|---|---|
| 1 | GTGATTTCGG GACTTCTATT CCTTCTAGTA AGACGAGAGG TTCCGACAGT |
| 51 | ACGTTCAGAG GAAATTCCCA GAGGGGTTTC TGTGACCCCT TCTGAAGAGC |
| 101 | CTGCTCTAGA GAAGGCTCAA AAAGAACCGG AGACAAAGAA AATTTTAGAT |
| 151 | CGGTTGCCGA AGGAATTGGA TCAGTTAGAT ACGTATATTC AGGAAGTGTT |
| 201 | TGCATGTTTA GAGAGGCTGA AGGATCCTAA GTACGAAGAT CGAGGTCTTT |
| 251 | TAACAGAGGC GAAGGAGAAA CTTCGAGTTT TTGACGTTGT TGAGAAAGAT |
| 301 | ATGATGTCAG AGTTTTTAGA CATACAACGA GTGTTGAATG AGGAAGCATA |
| 351 | TTATGTAGAA CATTGTCAAG ATCCCCTAGA GAATATAGCC TACGAGATTT |
| 401 | TCTCTTCCCA AGAGCTTCGT GATTACTACT GTGCAGGGGT GTGTGGGTAT |
| 451 | TTGCCTTCTG GGGATGCTCG AGCGGATCGA TTAAAGAGAT CAGTTAAGGA |
| 501 | GGTAATGGAT CGCTTTATGA GGGTGACCTG GAAATCTTGG GAGGCATCAG |
| 551 | TCATGTTGGA TCATAGCTAT GGGGTAGCGC GAGAGTTATT CAAGAAGGCA |
| 601 | GTAGGAGTAC TAGAGGAGAG TGTCTATAAA ATTCTGTTTA AGAGCTATAG |
| 651 | AGATGCGTTT TATGAATGTG AGAAGGCAAA GATCCAGAGG GATGGGCGTT |
| 701 | TCAAATGGTT ATAG |

The PSORT algorithm predicts cytoplasm (0.2817).

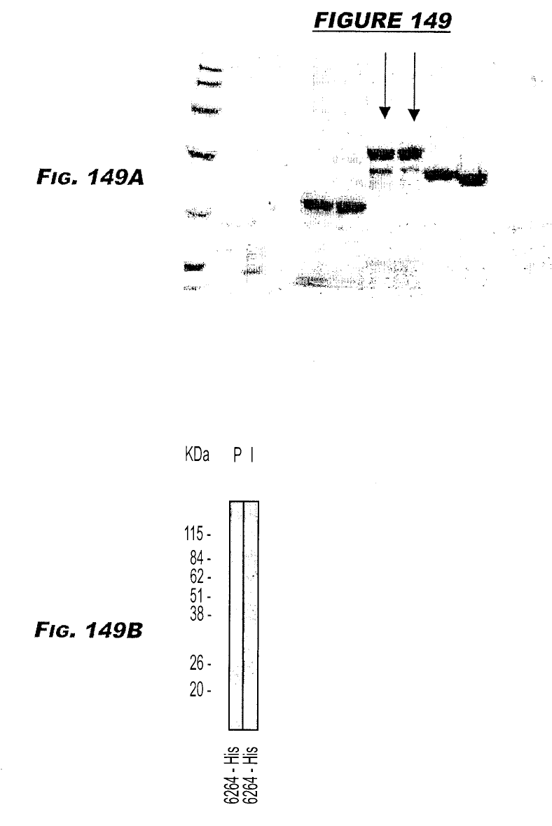

The protein was expressed in E. coli and purified as a his-tagged product (FIG. 149A). The recombinant protein was used to immunize mice, whose sera were used in a Western blot (FIG. 149B) and for FACS analysis.

These experiments show that cp6264 is a surface-exposed and immunoaccessible protein, and that it is a useful immunogen. These properties are not evident from the sequence alone.

### Example 150

The following C. pneumoniae protein (PID 4376266) was expressed <SEQ ID 299; cp6266>:

| | |
|---|---|
| 1 | MLLLISGALF LTLGIPGLSA AISFGLGIGL SALGGVLMIS GLLCLLVKRE |
| 51 | IPTVRPEEIP EGVSLAPSEE PALQAAQKTL AQLPKELDQL DTDIQEVFAC |
| 101 | LRKLKDSKYE SRSFLNDAKK ELRVFDFVVE DTLSEIFELR QIVAQEGWDL |
| 151 | NFLINGGRSL MMTAESESLD LFHVSKRLGY LPSGDVRGEG LKKSAKEIVA |
| 201 | RLMSLHCEIH KVAVAFDRNS YAMAEKAFAK ALGALEESVY RSLTQSYRDK |
| 251 | FLESERAKIP WNGHITWLRD DAKSGCAEKK LGMPRNVGRN LGKQSFG* |

The cp6266 nucleotide sequence <SEQ ID 300> is:

| | |
|---|---|
| 1 | ATGCTCTTAC TGATTTCAGG AGCTCTCTTT CTGACGTTAG GGATTCCAGG |
| 51 | ATTGAGTGCA GCAATTTCTT TTGGATTAGG CATCGGTCTC TCCGCATTAG |
| 101 | GAGGAGTGCT GATGATTTCG GGACTACTAT GTCTTTTAGT AAAACGAGAG |
| 151 | ATTCCGACAG TACGACCAGA AGAAATTCCT GAAGGGGTTT CGCTGGCTCC |
| 201 | TTCTGAGGAG CCAGCTCTAC AGGCAGCTCA GAAGACTTTA GCTCAGCTGC |
| 251 | CTAAGGAATT GGATCAGTTA GATACAGATA TTCAGGAAGT GTTCGCATGT |
| 301 | TTAAGAAAGC TGAAAGATTC TAAGTATGAA AGTCGAAGTT TTTTAAACGA |
| 351 | TGCTAAGAAG GAGCTTCGAG TTTTTGACTT TGTGGTTGAG GATACCCTCT |
| 401 | CGGAGATTTT CGAGTTGCGG CAGATTGTGG CTCAAGAGGG ATGGGATTTA |
| 451 | AACTTTTTGA TCAATGGGGG ACGAAGCCTC ATGATGACTG CAGAATCTGA |
| 501 | ATCGCTTGAT TTGTTTCATG TATCGAAGCG GCTAGGGTAT TTACCTTCTG |
| 551 | GGGATGTTCG AGGGGAGGGG TTAAAGAAAT CTGCGAAGGA GATAGTCGCT |
| 601 | CGTTTGATGA GCTTGCATTG CGAGATTCAC AAGGTGGCGG TAGCGTTTGA |
| 651 | TAGGAATTCC TATGCGATGG CAGAAAAGGC GTTTGCGAAA GCGTTGGGAG |
| 701 | CTTTAGAAGA GAGTGTGTAT CGGAGTCTGA CGCAGAGTTA TAGAGATAAA |
| 751 | TTTTTGGAGA GCGAGAGGGC GAAGATCCCA TGGAATGGGC ATATAACCTG |
| 801 | GTTAAGAGAT GATGCGAAGA GTGGGTGTGC TGAAAAGAAG CTCGGGATGC |
| 851 | CGAGGAACGT TGGAAGAAAT TTAGGAAAGC AGTCTTTTGG GTAG |

The PSORT algorithm predicts inner membrane (0.3590).

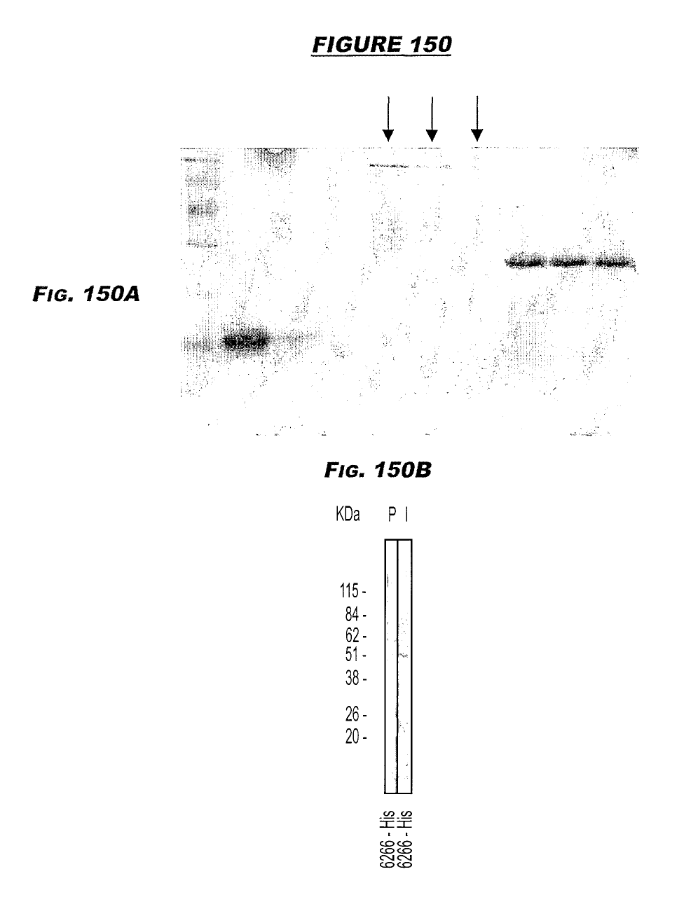

The protein was expressed in E. coli and purified as a his-tag product (FIG. 150A). The recombinant protein was used to immunize mice, whose sera were used in a Western blot (FIG. 150) and for FACS analysis.

These experiments show that cp6266 is a surface-exposed and immunoaccessible protein and that they it is a useful immunogen. These properties are not evident from the sequence alone.

### Example 151

The following C. pneumoniae protein (PID 4376895) was expressed <SEQ ID 301; cp6895>:

| | |
|---|---|
| 1 | MKIKKSFQYS LCQAKRFQNM LPNHFDPCLQ PVNLQLKQDR LAYGELIILL |
| 51 | SKYQQKTFSS LLKEETCSLN RAKQHLLYKI LRDFNTMQHL RSLGLNGWGE |
| 101 | TPMSPCL* |

The cp6895 nucleotide sequence <SEQ ID 302> is:

| | |
|---|---|
| 1 | ATGAAGATTA AAAAATCTTT TCAATACAGT TTATGCCAAG CAAAGAGATT |
| 51 | TCAGAACATG CTGCCAAACC ACTTTGATCC ATGTTTGCAG CCAGTGAATT |
| 101 | TACAACTCAA ACAAGACAGA TTGGCATACG GGGAGCTCAT CATATTGCTA |
| 151 | TCTAAATATC AACAAAAGAC CTTTTCCTCT TTGTTGAAGG AAGAAACATG |
| 201 | TTCTCTTAAT CGTGCGAAGC AGCACTTATT GTATAAGATT TTGAGAGATT |
| 251 | TTAATACTAT GCAGCATCTA AGGTCCCTCG GATTAAATGG TTGGGGAGAG |
| 301 | ATCCCTATGA GTCCTTGCCT CTAA |

The PSORT algorithm predicts cytoplasm (0.3264).

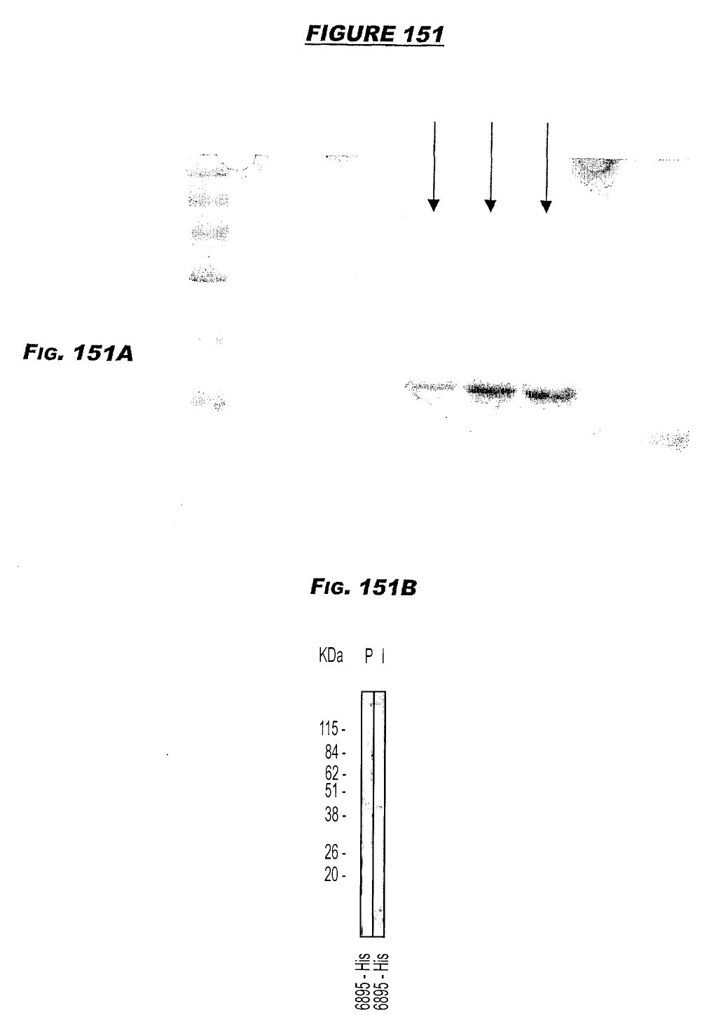

The protein was expressed in E. coli and purified as a his-tag product (FIG. 151A). The recombinant protein was used to immunize mice, whose sera were used in a Western blot (FIG. 151B) and for FACS analysis.

These experiments show that cp6895 is a surface-exposed and immunoaccessible protein and that it is a useful immunogen. These properties are not evident from the sequence alone.

### Example 152 and Example 153

The following C. pneumoniae protein (PID 4376282) was expressed <SEQ ID 303; cp6282>:

| | |
|---|---|
| 1 | MSLLNLPSSQ DSASEDSTSQ SQIFDPIRNR ELVSTPEEKV RQRLLSFLMH |
| 51 | KLNYPKKLII IEKELKTLFP LLMRKGTLIP KRRPDILIIT PPTYTDAQGN |
| 101 | THNLGDPKPL LLIECKALAV NQNALKQLLS YNYSIGATCI AMAGKHSQVS |
| 151 | ALFNPKTQTL DFYPGLPEYS QLLNYFISLN L* |

The cp6282 nucleotide sequence <SEQ ID 304> is:

| | |
|---|---|
| 1 | ATGTCCTTAT TGAACCTTCC CTCAAGCCAG GATTCTGCAT CTGAGGACTC |
| 51 | CACATCGCAA TCTCAAATCT TCGATCCCAT TAGAAATCGG GAGTTAGTTT |
| 101 | CTACTCCCGA AGAAAAAGTC CGCCAAAGGT TGCTCTCCTT CCTAATGCAT |
| 151 | AAGCTGAACT ACCCTAAGAA ACTCATCATC ATAGAAAAAG AACTCAAAAC |
| 201 | TCTTTTTCCT CTGCTTATGC GTAAAGGAAC CCTAATCCCA AAACGCCGCC |
| 251 | CAGATATTCT CATCATCACT CCCCCCACAT ACACAGACGC ACAGGGAAAC |
| 301 | ACTCACAACC TAGGCGACCC AAAACCCCTG CTACTTATCG AATGTAAGGC |
| 351 | CTTAGCCGTA AACCAAAATG CACTCAAACA ACTCCTTAGC TATAACTACT |
| 401 | CTATCGGAGC CACCTGCATT GCTATGGCAG GGAAACACTC TCAAGTGTCA |
| 451 | GCTCTCTTCA ATCCAAAAAC ACAAACTCTT GATTTTTATC CTGGCCTCCC |
| 501 | AGAGTATTCC CAACTCCTAA ACTACTTTAT TTCTTTAAAC TTATAG |

The PSORT algorithm predicts cytoplasm (0.362).

The following C. pneumoniae protein (PID 4377373) was also expressed <SEQ ID 305; cp7373>:

| | |
|---|---|
| 1 | MSTTTVKHFI HTASRWEPVL KEIVASNYWH AQWINTLSFL ENSGAKKISA |
| 51 | SEHPTEVKEE VLKHAAEEFR HGHYLKTQIS RISETSLPDY TSKNLLGGLL |
| 101 | TKYYLHLLDL RTCRVLENEY SLSGQTLKTA AYILVTYAIE LRASELYPLY |
| 151 | HDILKEAQSK ITVKSIILEE QGHLQEMERE LKDLPHGEEL LGYACQFEGE |
| 201 | LCLQFVERLE QMIFDPSSTF TKF* |

The cp7373 nucleotide sequence <SEQ ID 306> is:

| | |
|---|---|
| 1 | ATGTCTACAA CCACAGTAAA ACACTTTATC CACACAGCCT CTCGTTGGGA |
| 51 | GCCCGTTCTC AAAGAGATCG TAGCTTCCAA CTATTGGCAT GCACAATGGA |
| 101 | TAAATACCCT GTCCTTTTTA GAAAATAGTG GAGCAAAAAA AATCTCCGCA |
| 151 | AGTGAACATC CTACGGAGGT AAAGGAAGAA GTTTTAAAAC ATGCTGCTGA |
| 201 | AGAATTTCGT CATGGTCACT ATCTAAAAAC TCAGATTTCT AGAATCTCAG |
| 251 | AGACTTCTCT CCCTGACTAT ACATCTAAAA ATCTTCTGGG AGGCTTACTT |
| 301 | ACAAAATATT ACCTCCATCT TCTAGATTTA AGGACGTGCC GAGTACTGGA |
| 351 | AAATGAATAC TCCCTATCGG GACAAACGTT AAAAACTGCA GCGTATATTT |
| 401 | TAGTTACCTA CGCAATCGAA CTTCGTGCTT CTGAACTTTA TCCTCTGTAT |
| 451 | CACGATATTC TGAAAGAAGC TCAAAGTAAA ATAACGGTAA AATCCATTAT |
| 501 | CTTAGAAGAG CAAGGCCATC TGCAAGAGAT GGAACGTGAA CTTAAAGATC |
| 551 | TCCCCCACGG GGAGGAACTC TTAGGCTATG CTTGCCAATT CGAAGGGGAG |
| 601 | CTTTGCTTGC AGTTTGTAGA GAGATTAGAA CAAATGATCT TCGATCCTTC |
| 651 | CTCGACTTTT ACAAAGTTCT AG |

The PSORT algorithm predicts cytoplasm (0.1069).

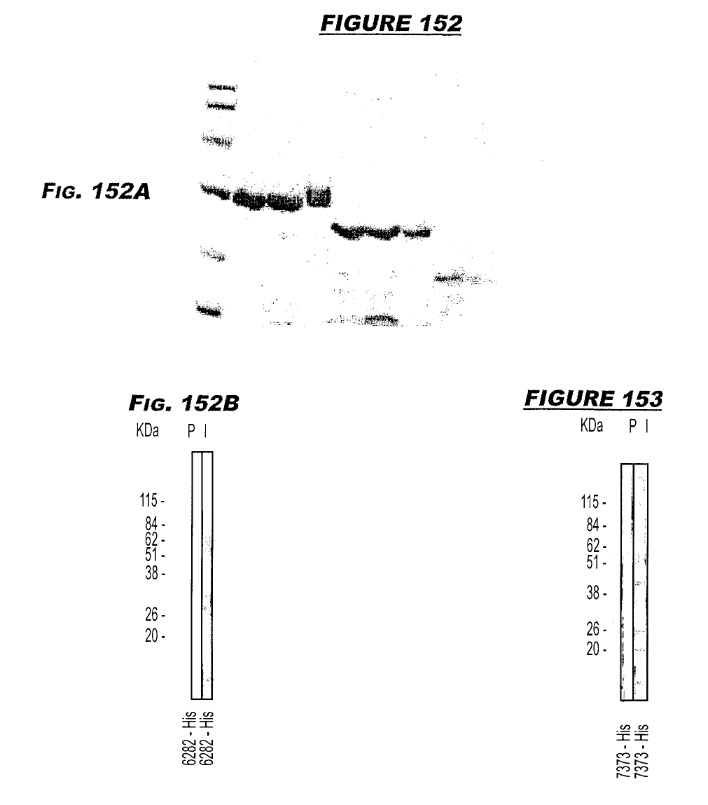

The proteins were expressed in E. coli and purified as his-tag products (FIG. 152A; 6282=lanes 8 & 9; 7373=lanes 2-4). The recombinant proteins were used to immunize mice, whose sera were used in Western blots (FIGS. 152B & 153) and for FACS analysis.

These experiments show that cp6282 & cp7373 are surface-exposed and immunoaccessible proteins and that they are useful immunogens. These properties are not evident from the sequence alone.

### Example 154, Example 155, Example 156, Example 157 and Example 158

The following C. pneumoniae protein (PID 4376412) was expressed <SEQ ID 307; cp6412>:

| | |
|---|---|
| 1 | MSSSEVVFQT VHGLGFGGLS SKSVVPFKKS LSDAPRVVCS ILVLTLGLGA |
| 51 | LVCGIAITCW CVPGVILMGG ICAIVLGAIS LALSLFWLWG LFSNCCGSKR |
| 101 | VLPGEGLLRD KLLDGGFSRA APSGMGLPGD GSPRASTPSC LEELQAEIQA |
| 151 | VTQAIDQMSD D* |

The cp6412 nucleotide sequence <SEQ ID 308> is:

| | |
|---|---|
| 1 | ATGAGCAGTT CGGAAGTTGT TTTCCAGACA GTTCATGGCC TTGGCTTTGG |
| 51 | TGGATTGTCT TCAAAAAGTG TTGTCCCTTT TAAGAAAAGT CTTTCGGATG |
| 101 | CGCCCCGTGT TGTGTGCTCG ATTTTAGTTT TGACTCTGGG GTTGGGAGCG |
| 151 | CTTGTTTGTG GTATTGCCAT TACTTGTTGG TGTGTCCCGG GAGTTATTTT |
| 201 | AATGGGGGGA ATTTGCGCTA TAGTTTTAGG TGCAATTTCT TTAGCTTTAA |
| 251 | GTCTATTTTG GTTGTGGGGT TTATTTTCTA ATTGTTGTGG TTCTAAGAGA |
| 301 | GTTTTACCGG GTGAGGGATT GCTACGGGAT AAGCTTTTAG ATGGTGGATT |
| 351 | TTCAAGAGCG GCACCTTCAG GAATGGGACT TCCGGGTGAT GGATCTCCAA |
| 401 | GAGCGTCAAC GCCATCTTGC CTAGAGGAAC TTCAAGCAGA GATACAGGCA |
| 451 | GTTACTCAAG CTATCGATCA GATGTCAGAT GATTGA |

The PSORT algorithm predicts inner membrane (0.4864).

The following C. pneumoniae protein (PID 4376431) was also expressed <SEQ ID 309; cp6431>:

| | |
|---|---|
| 1 | LRAGGSLVTT YPKEGQRLRS PEQLRVLDDL VQSYPNHLHA IELDCGAIPQ |
| 51 | DLIGATYIIT FADFSTYILS LRSYQANSPS DDTWGIWFGS IDDPVQAVIS |
| 101 | FLKDHGFALP STLAQDPLLC TNK* |

The cp6431 nucleotide sequence <SEQ ID 310> is:

| | |
|---|---|
| 1 | TTGCGAGCAG GAGGTAGTCT TGTTACAACA TACCCTAAGG AAGGTCAGAG |
| 51 | ATTGCGCTCC CCAGAACAGT TAAGAGTTCT GGATGATTTA GTGCAAAGCT |
| 101 | ATCCAAATCA CCTACATGCG ATTGAACTTG ATTGTGGTGC AATCCCTCAA |
| 151 | GATTTGATCG GAGCCACCTA TATCATCACG TTCGCCGATT TTTCCACCTA |
| 201 | TATTCTCTCT TTAAGAAGCT ACCAAGCCAA TTCTCCCTCC GATGATACAT |
| 251 | GGGGGATTTG GTTTGGATCT ATTGACGATC CTGTTCAAGC AGTCATATCA |
| 301 | TTTTTAAAAG ATCATGGATT TGCTCTTCCC TCGACCTTAG CTCAAGATCC |
| 351 | TTTGCTTTGT ACTAACAAGT AA |

The PSORT algorithm predicts cytoplasm (0.2115).

The following C. pneumoniae protein (PID 4376443) was also expressed <SEQ ID 311; cp6443>:

| | |
|---|---|
| 1 | MIMTTISNSP SPALNPELSL IPPPTLVSSG TQTSLAYTIP AQGRRSTLRI |
| 51 | ILDIFIIILG LATIISTFIV IFFLNGLNLL STPSIISSSC LIIVGLLFLI |
| 101 | MGLYFMISSL DQGLVGLLQK ELSQAEEREE EYIQEIEALR GAPRAESPTE |
| 151 | SPSTWL* |

The cp6443 nucleotide sequence <SEQ ID 312> is:

| | |
|---|---|
| 1 | ATGATTATGA CTACTATATC TAACTCACCC TCCCCTGCAT TGAATCCCGA |
| 51 | ACTTTCCCTT ATTCCTCCAC CAACACTTGT ATCTTCAGGT ACGCAAACAT |
| 101 | CTCTAGCTTA TACGATCCCC GCACAAGGAC GAAGATCCAC CCTACGTATT |
| 151 | ATATTAGATA TATTCATTAT CATTCTTGGT TTAGCTACGA TCATTTCTAC |
| 201 | CTTTATTGTT ATTTTCTTTT TAAATGGGCT GAACTTGCTC TCGACCCCAT |
| 251 | CTATTATCTC TTCGTCATGT TTAATCATTG TTGGATTGCT TTTTTTGATT |
| 301 | ATGGGGTTAT ATTTCATGAT CTCGAGTTTG GATCAGGGGC TTGTAGGCCT |
| 351 | TCTGCAAAAG GAACTCTCTC AAGCCGAAGA AAGAGAAGAA GAGTATATCC |
| 401 | AGGAAATCGA AGCTTTAAGA GGAGCTCCTA GAGCAGAATC TCCCACAGAG |
| 451 | TCTCCTAGTA CCTGGTTATG A |

The PSORT algorithm predicts inner membrane (0.5585).

The following C. pneumoniae protein (PID 4376496) was also expressed <SEQ ID 313; cp6496>:

| | |
|---|---|
| 1 | MLIGRYSSDD QFTEATKNTP TIIKLGFVRD NLEGLTNPIS EIVSETSSSI |
| 51 | KDSVLRSLPI LGSILGCARL YSTLSTNDPL DETQEKIWHT IFGALETLGL |
| 101 | GILILLFKII FVILHCIFHL VIGFCK* |

The cp6496 nucleotide sequence <SEQ ID 314> is:

| | |
|---|---|
| 1 | ATGCTAATAG GCAGATACAG TAGTGATGAC CAATTCACTG AAGCAACAAA |
| 51 | AAACACCCCA ACCATAATTA AGCTAGGTTT TGTTAGAGAT AATCTCGAGG |
| 101 | GATTAACGAA CCCTATCTCT GAAATCGTCT CGGAAACCTC CTCTTCTATT |
| 151 | AAAGATTCCG TTCTTCGCTC TCTTCCTATT TTAGGGTCCA TTTTAGGATG |
| 201 | CGCCCGACTT TACAGCACAC TCTCTACAAA TGATCCTCTT GACGAAACTC |
| 251 | AAGAAAAGAT TTGGCACACT ATATTTGGAG CCTTAGAAAC CTTAGGCTTA |
| 301 | GGGATTCTCA TCCTCTTATT TAAAATTATT TTTGTTATAT TACACTGCAT |
| 351 | ATTTCATCTA GTTATTGGGT TCTGCAAATA A |

The PSORT algorithm predicts inner membrane (0.5989).

The following C. pneumoniae protein (PID 4376654) was also expressed <SEQ ID 315; cp6654>:

| | |
|---|---|
| 1 | MKTKMNSRKK AGQWAIFNSP TPGVSSTLVL AWTPWGYYDK DVQDILERKD |
| 51 | PMSSSLSEKD SKEFLKNLFV DLLENGFTSV HIHAEEAFTP LDHTGKPHFK |
| 101 | RDNVYLPGKL LGALNEAAVQ ANVSADTQFT LFLTQDECNP FHDKKRG* |

The cp6654 nucleotide sequence <SEQ ID 316> is:

| | |
|---|---|
| 1 | ATGAAAACTA AAATGAACTC TAGAAAAAAA GCAGGTCAAT GGGCAATTTT |
| 51 | CAATTCTCCA ACTCCTGGTG TCAGTTCAAC TTTAGTTTTA GCATGGACTC |
| 101 | CTTGGGGTTA TTACGACAAG GATGTACAAG ATATCTTAGA AAGAAAAGAT |
| 151 | CCGATGAGCT CTTCGCTTTC TGAAAAAGAC TCAAAGGAGT TCTTGAAAAA |
| 201 | TCTGTTTGTA GATCTCTTAG AAAATGGCTT CACATCAGTA CATATTCACG |
| 251 | CAGAAGAAGC TTTCACTCCT CTTGATCATA CCGGGAAACC TCACTTTAAA |
| 301 | AGAGACAATG TGTACTTACC CGGAAAGTTG TTAGGCGCCT TGAATGAGGC |
| 351 | TGCGGTACAA GCCAATGTAA GTGCGGATAC TCAATTTACA TTGTTCCTTA |
| 401 | CTCAAGATGA GTGCAATCCT TTTCATGATA AGAAAAGAGG TTAA |

The PSORT algorithm predicts cytoplasm (0.0730).

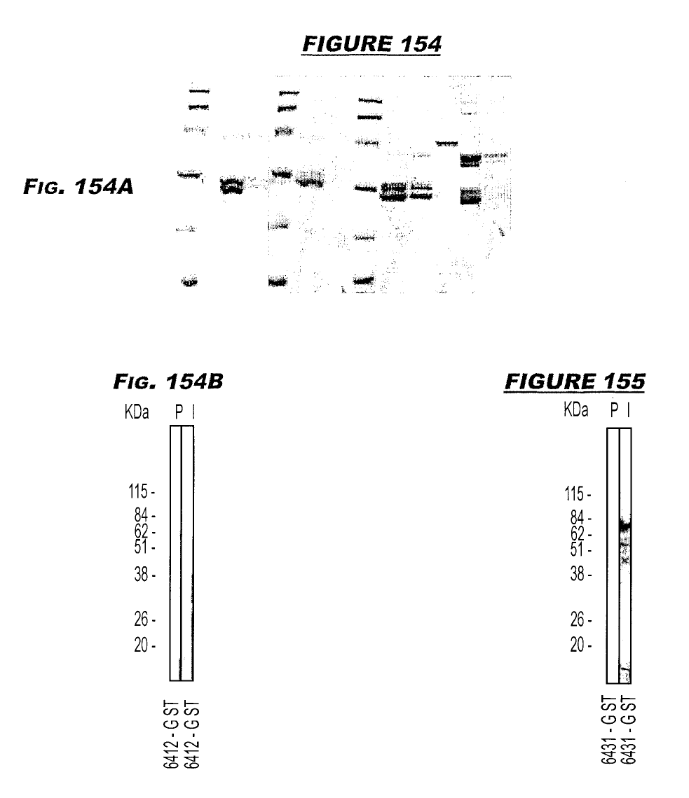

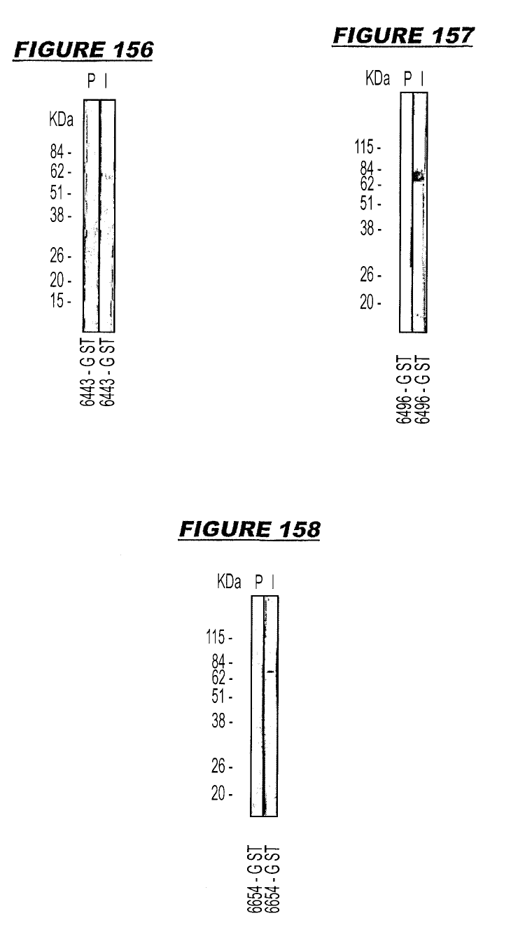

The proteins were expressed in E. coli and purified as his-tag products (FIG. 154A; 6412=lanes 2-3; 6431=lanes 11-12; 6443=lanes 5-6; 6496=lanes 8-9; 6654=lane 10; markers in lanes 1, 4, 7). The recombinant proteins were used to immunize mice, whose sera were used in Western blots (FIGS. 154B, 155, 156, 157 & 158) and for FACS analysis.

These experiments show that cp6412, cp6431, cp6443, cp6496 & cp6654 are surface-exposed and immunoaccessible proteins and that they are useful immunogens. These properties are not evident from their sequences alone.

### Example 159 and Example 160

The following C. pneumoniae protein (PID 4376477) was expressed <SEQ ID 317; cp6477>:

| | |
|---|---|
| 1 | LLKFFLVCEE LCILTVATHR ALLETPLALS FFKELKTKYV YRAKDILQLH |
| 51 | NYKGFTILNT SPLCS* |

The cp6477 nucleotide sequence <SEQ ID 318> is:

| | |
|---|---|
| 1 | TTGCTAAAGT TCTTTCTAGT ATGTGAAGAG TTATGTATAC TTACTGTTGC |
| 51 | TACACATAGA GCTCTCTTAG AAACTCCTTT AGCTCTATCA TTTTTTAAAG |
| 101 | AACTTAAGAC AAAATATGTC TACAGGGCGA AAGACATACT ACAACTACAT |
| 151 | AACTATAAAG GATTTACTAT CCTTAATACA TCACCGTTAT GTTCTTAA |

The PSORT algorithm predicts inner membrane (0.128).

The following C. pneumoniae protein (PID 4376435) was also expressed <SEQ ID 319; cp6435>:

| | |
|---|---|
| 1 | LWSHFPRGFF MLPFCPTILL AKPFLNSENY GLERLAATVD SYFDLGQSQI |
| 51 | VFLSKQDQGI TVEELSAKDR KFKPGSMNCT LYTEDPILPA HNSFSNCSDI |
| 101 | QMRTPISPIH * |

The cp6435 nucleotide sequence <SEQ ID 320> is:

| | |
|---|---|
| 1 | TTGTGGTCGC ATTTCCCAAG AGGATTTTTT ATGCTCCCTT TTTGCCCTAC |
| 51 | CATCCTTCTT GCTAAACCTT TTTTAAATAG CGAGAATTAC GGCTTAGAAC |
| 101 | GTTTAGCTGC AACCGTAGAT TCTTATTTTG ATCTGGGACA GTCTCAAATA |
| 151 | GTCTTCCTAA GCAAACAGGA TCAAGGAATC ACTGTGGAAG AATTGAGTGC |
| 201 | TAAAGATAGG AAATTCAAGC CAGGCTCTAT GAACTGTACA CTGTACACTG |
| 251 | AAGATCCTAT CTTACCTGCT CATAATTCCT TTAGTAATTG CTCTGATATT |
| 301 | CAAATGCGTA CTCCGATTAG CCCTATACAT TAA |

The PSORT algorithm predicts periplasmic space (0.4044).

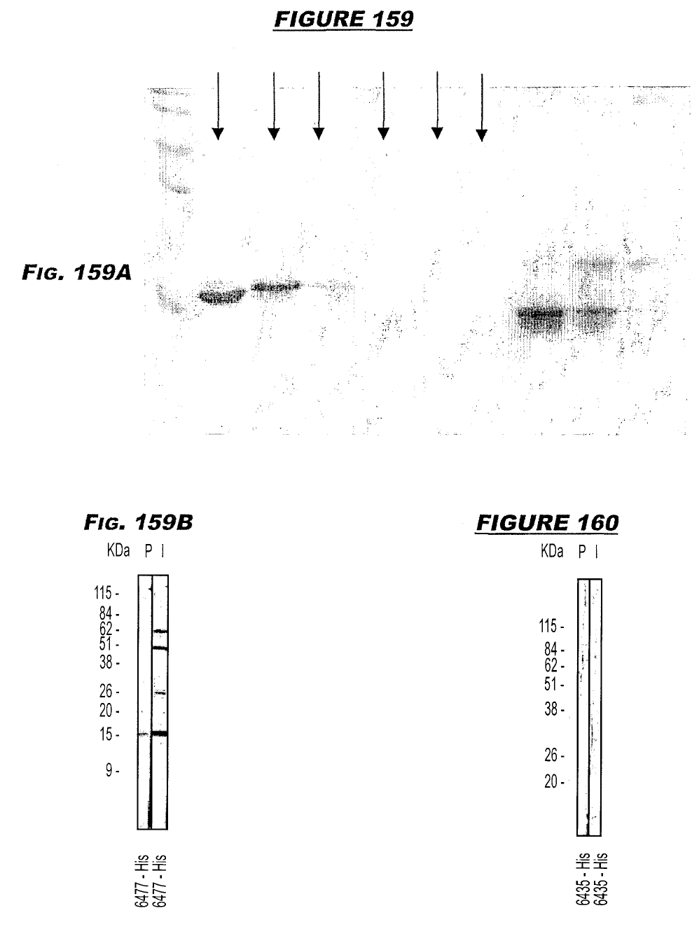

The proteins were expressed in E. coli and purified as his-tag products (FIG. 159A; 6435=lanes 2-4; 6477=lanes 5-7). The recombinant proteins were used to immunize mice, whose sera were used in Western blots (FIGS. 159B & 160) and for FACS analysis.

These experiments show that cp6477 & cp6435 are surface-exposed and immunoaccessible proteins and that they are useful immunogens. These properties are not evident from the sequences alone.

### Example 161 and Example 162 and Example 163

The following C. pneumoniae protein (PID 4376441) was expressed <SEQ ID 321; cp6441>:

| | |
|---|---|
| 1 | VEAGANVLVI DTAHAHSKGV FQTVLEIKSQ FPQISLVVGN LVTAEAAVSL |
| 51 | AEIGVDAVKV GIGPGSICTT RIVSGVGYPQ ITAITNVAKA LKNSAVTVIA |
| 101 | DGRIRYSGDV VKALAAGADC VMLGSLLAGT DEAPGDIVSI DEKLFKRYRG |
| 151 | MGSLGAMKQG SADRYFQTQG QKKLVPGGVE GLVAYKGSVH DVLYQILGGI |
| 201 | RSGMGYVGAE TLKDLKTKAS FVRITESGRA ESHIHNIYKV QPTLNY |

The cp6441 nucleotide sequence <SEQ ID 322> is:

| | |
|---|---|
| 1 | GTGGAAGCTG GAGCAAATGT TCTAGTCATT GACACAGCTC ATGCACACTC |
| 51 | TAAAGGAGTA TTCCAAACAG TTTTAGAAAT AAAATCCCAG TTCCCACAAA |
| 101 | TTTCTTTAGT TGTAGGGAAT CTTGTTACAG CTGAAGCCGC AGTTTCCTTA |
| 151 | GCTGAGATTG GAGTTGACGC TGTAAAGGTA GGTATTGGCC CAGGATCTAT |
| 201 | CTGTACAACT AGAATCGTTT CAGGGGTCGG TTATCCACAA ATTACTGCCA |
| 251 | TTACAAACGT AGCAAAAGCT CTTAAAAACT CTGCCGTGAC TGTAATTGCT |
| 301 | GATGGGAGAA TCCGCTATTC TGGAGATGTG GTAAAAGCAT TAGCAGCAGG |
| 351 | AGCAGACTGT GTCATGCTAG GAAGTTTGCT TGCAGGGACT GATGAAGCTC |
| 401 | CTGGGGATAT CGTTTCTATC GATGAGAAGC TTTTTAAAAG GTACCGCGGC |
| 451 | ATGGGATCTT TAGGCGCTAT GAAACAAGGA AGTGCTGACC GGTATTTTCA |
| 501 | AACACAGGGA CAGAAAAAGC TGGTTCCTGG GGGAGTTGAA GGACTAGTCG |
| 551 | CTTATAAAGG CTCTGTCCAC GATGTCCTCT ATCAAATTTT AGGAGGAATA |
| 601 | CGCTCAGGTA TGGGGTATGT TGGAGCTGAA ACTCTCAAAG ATTTAAAAAC |
| 651 | TAAGGCTTCC TTTGTTCGAA TTACTGAATC TGGAAGAGCT GAAAGTCATA |
| 701 | TTCATAATAT TTACAAAGTT CAACCAACCT TAAATTATTA A |

The PSORT algorithm predicts bacterial inner membrane (0.132).

The following C. pneumoniae protein (PID 4376748) was also expressed <SEQ ID 323; cp6748>:

| | |
|---|---|
| 1 | LFSEGTALNL FRIFAPLRNR VTTEYSRARQ PDLHRIAIVY IGVLDSESSK |
| 51 | ILERLISYMS CIYSESQMYL RFFMGKNVNQ SAVLSKLHVE NLHIRCGFFS |
| 101 | EDAVPESEPF DLSIYVHTDR SCPLPTKKRS SSWELQTVEL PESIYPQSEF |
| 151 | LLMRPRMLS* |

The cp6748 nucleotide sequence <SEQ ID 324> is:

| | |
|---|---|
| 1 | TTGTTCTCTG AGGGGACAGC TCTAAATTTA TTTCGTATAT TTGCTCCACT |
| 51 | ACGCAACCGT GTGACTACAG AATACAGTCG TGCTAGGCAA CCCGACCTAC |
| 101 | ATAGAATTGC CATCGTCTAT ATAGGAGTTC TCGATTCAGA AAGTTCCAAG |
| 151 | ATCCTAGAGC GGCTAATCTC TTATATGAGT TGTATCTATT CTGAATCGCA |
| 201 | AATGTATTTA AGATTCTTTA TGGGCAAGAA TGTAAATCAA AGTGCTGTAC |
| 251 | TCTCAAAATT ACATGTAGAA AATCTGCACA TCCGTTGTGG GTTTTTCAGC |
| 301 | GAGGATGCTG TTCCAGAGAG TGAGCCCTTC GATCTCTCCA TCTACGTGCA |
| 351 | CACAGATCGT AGCTGTCCTC TCCCTACGAA AAAACGGAGC AGCTCCTGGG |
| 401 | AACTCCAAAC TGTAGAACTC CCAGAGTCAA TATATCCACA GTCGGAATTC |
| 451 | CTATTGATGA GACCTCGAAT GCTTTCGTAG |

The PSORT algorithm predicts cytoplasm (0.170).

The following C. pneumoniae protein (PID 4376881) was also expressed <SEQ ID 325; cp6881>:

| | |
|---|---|
| 1 | MRPHRKHVSS KSLALKQSAS THVEITTKAF RLSMPLKQLI LEKSDHLPPM |
| 51 | ETIRVVLTSH KDKLGTEVHV VASHGKEILQ TKVHNANPYT AVINAFKKIR |
| 101 | TMANKHSNKR KDRTKHDLGL AAKEERIAIQ EEQEDRLSNE WLPVEGLDAW |
| 151 | DSLKTLGYVP ASAKKKISKK KMSIRMLSQD EAIRQLESAA ENFLIFLNEQ |
| 201 | EHKIQCIYKK HDGNYVLIEP SLKPGFCI* |

The cp6881 nucleotide sequence <SEQ ID 326> is:

| | |
|---|---|
| 1 | ATGAGACCTC ATCGTAAACA CGTATCATCT AAAAGCTTAG CTTTAAAGCA |
| 51 | ATCTGCATCA ACTCATGTAG AGATCACAAC AAAAGCCTTT CGTCTCTCTA |
| 101 | TGCCTCTAAA ACAGCTGATC CTAGAGAAAA GCGACCACCT CCCCCCTATG |
| 151 | GAAACAATCC GTGTGGTGCT AACCTCTCAT AAAGATAAGC TAGGCACCGA |
| 201 | GGTGCATGTT GTAGCTTCTC ATGGCAAAGA AATCCTTCAA ACTAAGGTTC |
| 251 | ATAACGCAAA CCCATACACT GCAGTGATCA ATGCTTTTAA GAAAATCCGC |
| 301 | ACCATGGCAA ATAAGCACTC CAATAAACGT AAAGACAGGA CAAAACATGA |
| 351 | TCTAGGTCTT GCAGCAAAAG AAGAACGTAT CGCAATACAG GAAGAACAAG |
| 401 | AAGATCGCCT TAGCAACGAG TGGCTTCCTG TCGAAGGCCT CGATGCCTGG |
| 451 | GATTCTCTAA AAACTCTTGG GTATGTTCCC GCATCAGCGA AAAAGAAGAT |
| 501 | CTCCAAGAAA AAGATGAGCA TTCGTATGCT ATCTCAAGAC GAGGCTATCC |
| 551 | GCCAGCTAGA GTCTGCCGCA GAAAACTTCC TGATCTTCTT GAACGAGCAA |
| 601 | GAGCATAAAA TCCAATGCAT TTATAAAAAA CATGACGGCA ACTATGTCCT |
| 651 | TATTGAACCT TCCCTCAAGC CAGGATTCTG CATCTGA |

The PSORT algorithm predicts cytoplasm (0.249).

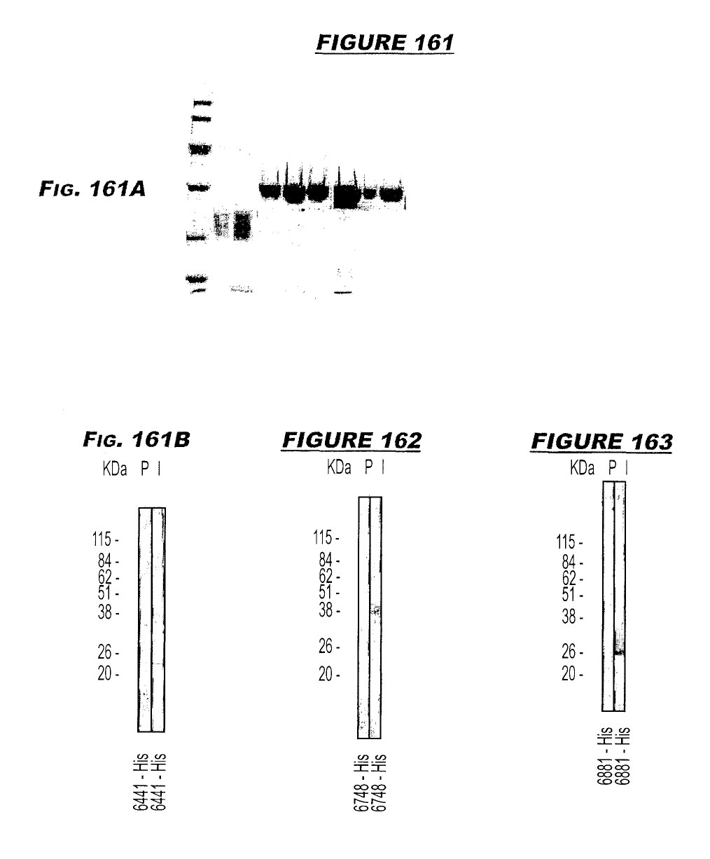

The proteins were expressed in E. coli and purified as his-tag products (FIG. 161A; 6441=lanes 7-9; 6748=lanes 2-3; 6881=lanes 4-6). The recombinant protein was used to immunize mice, whose sera were used in Western blots (FIGS. 161B, 162 & 163) and for FACS analysis.

These experiments show that cp6441, cp6748 & cp6881 are surface-exposed and immunoaccessible proteins and that they are useful immunogens. These properties are not evident from the sequence alone.

### Example 164 and Example 165 Example 166

The following C. pneumoniae protein (PID 4376444) was expressed <SEQ ID 327; cp6444>:

| | |
|---|---|
| 1 | MEQPNCVIQD TTTVLYALNS FDPRLSDDTH RLGKQSPLEA ENALGEFIEG |
| 51 | LDTNSFPLEE VAIPILPGYH PKFYLSFIDR DDQGVHYEVL DGVFLKTVAA |
| 101 | CIIENSFLTD SMSPELLSEV KEALKR* |

The cp6444 nucleotide sequence <SEQ ID 328> is:

| | |
|---|---|
| 1 | ATGGAGCAAC CCAATTGTGT GATTCAGGAT ACTACAACTG TTTTGTATGC |
| 51 | CTTAAATAGC TTTGATCCTA GACTTAGTGA TGACACTCAC AGACTTGGGA |
| 101 | AGCAATCACC TCTTGAAGCA GAAAATGCTC TTGGAGAATT TATTGAAGGT |
| 151 | TTGGATACAA ATAGCTTTCC TTTAGAGGAA GTTGCCATTC CCATCCTGCC |
| 201 | AGGTTATCAC CCTAAGTTTT ATTTATCTTT CATAGATAGG GACGATCAAG |
| 251 | GTGTCCACTA TGAAGTTTTA GATGGCGTAT TTTTAAAGAC AGTCGCTGCT |
| 301 | TGTATTATAG AGAACTCCTT CTTAACTGAT TCTATGAGCC CGGAGCTTCT |
| 351 | CAGCGAAGTT AAGGAAGCTC TGAAACGATG A |

The PSORT algorithm predicts cytoplasm (0.2031).

The following C. pneumoniae protein (PID 4376413) was also expressed <SEQ ID 329; cp6413>:

| | |
|---|---|
| 1 | MAVQSIKEAV TSAATSVGCV NCSREAIPAF NTEERATSIA RSVIAAIIAV |
| 51 | VAISLLGLGL VVLAGCCPLG MAAGAITMLL GVALLAWAIL ITLRLLNIPK |
| 101 | AEIPSPGNNG EPNERNSATP PLEGGVAGEA GRGGGSPLTQ LDLNSGAGS* |

The cp6413 nucleotide sequence <SEQ ID 330> is:

| | |
|---|---|
| 1 | ATGGCTGTTC AATCTATAAA AGAAGCCGTA ACATCAGCCG CAACATCAGT |
| 51 | AGGATGTGTA AACTGTTCTA GAGAGGCTAT ACCAGCATTT AATACAGAGG |
| 101 | AGAGAGCAAC GAGTATTGCT AGATCTGTTA TAGCAGCTAT CATTGCTGTT |
| 151 | GTAGCTATCT CCTTACTCGG ACTAGGTCTT GTAGTTCTTG CTGGTTGCTG |
| 201 | TCCTTTAGGA ATGGCTGCGG GTGCTATAAC AATGCTGCTG GGTGTAGCAT |
| 251 | TATTAGCTTG GGCAATACTG ATTACTTTGA GACTGCTTAA TATACCTAAG |
| 301 | GCTGAAATAC CGAGTCCAGG GAACAACGGT GAGCCTAATG AAAGAAATTC |
| 351 | AGCAACTCCT CCTCTAGAGG GTGGTGTTGC AGGAGAAGCC GGTCGCGGCG |
| 401 | GGGGGTCACC TTTAACCCAA CTTGATCTCA ATTCAGGGGC GGGAAGTTAG |

The PSORT algorithm predicts inner membrane (0.6180).

The following C. pneumoniae protein (PID 4377391) was also expressed <SEQ ID 331; cp7391>:

| | |
|---|---|
| 1 | MMLRVIELPL LPIKQALEKA FVQYNSYKAK LTKVEPCFRE SPAYITSEER |
| 51 | LQSLDQTLER AYKEYQKRFQ EPSRLESEVS GCREHLREQV KQFETQGLDL |
| 101 | IKEELIFVSD VLFRKMVSCL VSTVHVPFME FYYEYFELHR LRLRAQWMAN |
| 151 | AEIYSKVRKA FPEMLKETLE KAKAPREEEY WLLCEERKSK EKRLILNKIE |
| 201 | AAQQRVKDLE PPPIKETGKQ KRKKEYSFFI RLKS* |

The cp7391 nucleotide sequence <SEQ ID 332> is:

| | |
|---|---|
| 1 | ATGATGCTTC GTGTCATAGA GCTTCCACTA CTTCCTATAA AGCAAGCGTT |
| 51 | GGAGAAGGCT TTTGTACAAT ATAATAGCTA CAAAGCGAAG TTAACCAAGG |
| 101 | TAGAACCTTG CTTTAGAGAG AGCCCTGCCT ATATAACTAG CGAAGAGCGA |
| 151 | CTCCAGAGTT TGGATCAGAC TTTAGAACGT GCGTACAAAG AGTACCAGAA |
| 201 | GAGATTCCAG GAGCCTTCAC GTTTGGAATC GGAAGTAAGT GGATGTAGAG |
| 251 | AGCATCTTAG AGAGCAGGTA AAACAATTTG AAACTCAAGG ACTAGACTTG |
| 301 | ATCAAAGAAG AGCTTATTTT TGTTAGTGAT GTGTTATTCC GAAAAATGGT |
| 351 | CAGTTGTCTA GTGTCGACAG TGCATGTTCC CTTTATGGAG TTTTATTATG |
| 401 | AGTATTTTGA GTTGCATAGA TTGAGGTTGC GGGCCCAATG GATGGCGAAT |
| 451 | GCCGAGATTT ATAGCAAAGT TAGAAAAGCA TTCCCAGAGA TGTTGAAGGA |
| 501 | GACCTTAGAA AAAGCTAAGG CTCCCAGAGA AGAAGAGTAT TGGTTACTTT |
| 551 | GCGAGGAGAG AAAGAGTAAG GAGAAGCGTT TGATTCTCAA CAAGATAGAG |
| 601 | GCAGCTCAGC AGCGGGTAAA AGATTTAGAA CCTCCTCCTA TTAAAGAGAC |
| 651 | AGGGAAACAG AAACGGAAGA AAGAATATTC GTTTTTCATT CGATTAAAAT |
| 701 | CGTGA |

The PSORT algorithm predicts inner membrane (0.1489).

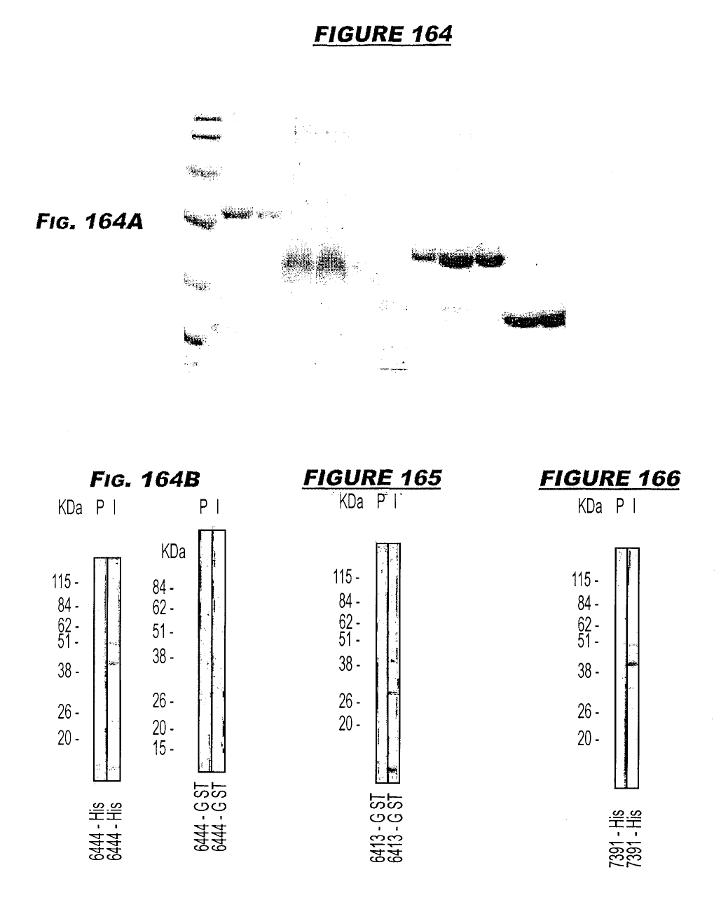

The proteins were expressed in E. coli and purified as his-tag and GST-fusion products (FIG. 164A; 6444=lanes 11-12; 7391=lanes 2-3; 6413=lanes 4-6). The recombinant protein was used to immunize mice, whose sera were used in Western blots (FIGS. 164B, 165 & 166) and for FACS analysis.

These experiments show that cp6444, cp6413 & cp7391 are surface-exposed and immunoaccessible proteins and that they are useful immunogens. These properties are not evident from the sequence alone.

### Example 167, Example 168, Example 169 and Example 170

The following C. pneumoniae protein (PID 4376463) was expressed <SEQ ID 333; cp6463>:

| | |
|---|---|
| 1 | MKKKVTIDEA LKEILRLEGA ATQEELCAKL LAQGFATTQS SVSRWLRKIQ |
| 51 | AVKVAGERGA RYSLPSSTEK TTTRHLVLSI RHNASLIVIR TVPGSASWIA |
| 101 | ALLDQGLKDE ILGTLAGDDT IFVTPIDEGR LPLLMVSIAN LLQVFLD* |

The cp6463 nucleotide sequence <SEQ ID 334> is:

| | |
|---|---|
| 1 | ATGAAAAAAA AAGTAACTAT AGATGAGGCT TTAAAAGAAA TTTTACGTCT |
| 51 | TGAAGGAGCG GCAACTCAGG AGGAATTATG TGCAAAACTC TTAGCTCAAG |
| 101 | GTTTTGCTAC AACCCAGTCG TCTGTATCTC GTTGGCTACG AAAGATTCAG |
| 151 | GCTGTAAAGG TTGCTGGAGA GCGTGGTGCT CGTTATTCTT TACCCTCTTC |
| 201 | AACAGAGAAG ACCACGACCC GTCATTTGGT GCTCTCTATT CGCCATAACG |
| 251 | CCTCTCTTAT TGTAATTCGT ACGGTTCCTG GTTCAGCTTC TTGGATCGCT |
| 301 | GCTTTGTTAG ATCAAGGGCT CAAAGATGAA ATTCTTGGAA CTTTGGCAGG |
| 351 | AGATGACACG ATTTTTGTCA CTCCTATAGA TGAAGGGAGG CTCCCATTGT |
| 401 | TGATGGTTTC GATTGCAAAT TTACTGCAAG TTTTCTTGGA TTAA |

The PSORT algorithm predicts inner membrane (0.1510).

The following C. pneumoniae protein (PID 4376540) was also expressed <SEQ ID 335; cp6540>:

| | |
|---|---|
| 1 | MSQCQSSSTS TWEWMKSFVP NWKNPTPPLS PIPSEDEFIL AYEPFVLPKT |
| 51 | DPENAQANPP GTSTPNVENG IDDLNPLLGQ PNEQNNANNP GTSGSNPTSL |
| 101 | PAPERLPETE ENSQEEEQGS QNNEDLIG* |

The cp6540 nucleotide sequence <SEQ ID 336> is:

| | |
|---|---|
| 1 | ATGTCTCAAT GTCAGAGTAG CAGTACATCT ACCTGGGAAT GGATGAAATC |
| 51 | TTTTGTGCCA AACTGGAAGA ATCCAACTCC CCCCTTATCT CCTATACCTT |
| 101 | CTGAGGACGA ATTTATATTA GCATACGAGC CATTTGTTCT ACCGAAAACA |
| 151 | GATCCAGAAA ACGCACAAGC TAATCCTCCA GGCACATCTA CACCGAATGT |
| 201 | AGAAAACGGG ATCGATGATC TCAACCCTCT TCTGGGGCAA CCCAACGAAC |
| 251 | AAAACAATGC CAACAATCCA GGAACTTCTG GATCTAATCC TACATCTCTA |
| 301 | CCCGCCCCCG AACGACTCCC TGAAACTGAA GAGAACAGCC AAGAAGAAGA |
| 351 | ACAAGGATCT CAAAATAATG AGGATCTTAT AGGATAA |

The PSORT algorithm predicts cytoplasm (0.3086).

The following C. pneumoniae protein (PID 4376743) was also expressed <SEQ ID 337; cp6743>:

| | |
|---|---|
| 1 | LREEGSVSFR EYFRAYMCDK IVAQKNFLFT LDAVIKQAGW RSQEKLNLFY |
| 51 | VESQALGREI KVSLEEYIQS MVGILGSQRT KKSFKFSVDF TPLEQALQER |
| 101 | CSSDDDEDAT ATSTATGATA SPTDMHEDE* |

The cp6743 nucleotide sequence <SEQ ID 338> is:

| | |
|---|---|
| 1 | TTGAGAGAAG AAGGTAGTGT TTCTTTCAGA GAATATTTCA GAGCCTATAT |
| 51 | GTGTGATAAA ATCGTGGCAC AGAAGAACTT CTTATTTACT TTAGACGCTG |
| 101 | TAATTAAACA GGCCGGTTGG AGATCACAAG AGAAACTCAA TTTATTTTAT |
| 151 | GTTGAAAGTC AGGCTTTAGG AAGAGAAATC AAAGTCAGCT TAGAGGAATA |
| 201 | TATTCAGAGT ATGGTCGGGA TTTTGGGATC TCAGAGAACC AAGAAAAGCT |
| 251 | TTAAGTTTTC TGTCGACTTT ACCCCTTTAG AGCAGGCTCT ACAAGAAAGA |
| 301 | TGCTCTTCTG ATGATGACGA AGATGCAACA GCAACTTCGA CCGCTACAGG |
| 351 | GGCAACAGCA TCTCCGACTG ACATGCACGA AGATGAGTAA |

The PSORT algorithm predicts cytoplasm (0.2769).

The following C. pneumoniae protein (PID 4377041) was also expressed <SEQ ID 339; cp7041>:

| | |
|---|---|
| 1 | MLMMLMMIIG ITGGSGAGKT TLTQNIKEIF GEDVSVICQD NYYKDRSHYT |
| 51 | PEERANLIWD HPDAFDNDLL ISDIKRLKNN EIVQAPVFDF VLGNRSKTEI |
| 101 | ETIYPSKVIL VEGILVFENQ ELRDLMDIRI FVDTDADERI LRRMVRDVQE |
| 151 | QGDSVDCIMS RYLSMVKPMH EKFIEPTRKY ADIIVHGNYR QNVVTNILSQ |
| 201 | KIKNHLENAL ESDETYYMVN SK* |

The cp7041 nucleotide sequence <SEQ ID 340> is:

| | |
|---|---|
| 1 | ATGTTGATGA TGCTTATGAT GATTATTGGA ATTACAGGAG GTTCTGGAGC |
| 51 | TGGGAAAACC ACCCTAACCC AAAACATTAA AGAAATTTTC GGTGAGGATG |
| 101 | TGAGTGTTAT CTGCCAAGAT AATTATTACA AAGATAGATC TCATTATACT |
| 151 | CCTGAAGAAC GTGCCAATTT AATTTGGGAT CATCCGGACG CCTTTGATAA |
| 201 | TGACTTATTA ATTTCAGACA TAAAACGTCT AAAAAATAAT GAGATTGTCC |
| 251 | AAGCCCCAGT TTTTGATTTT GTTTTAGGTA ATCGATCTAA AACGGAGATA |
| 301 | GAAACGATCT ATCCATCTAA AGTTATTCTT GTTGAAGGTA TTCTGGTCTT |
| 351 | TGAAAATCAA GAACTTAGAG ATCTTATGGA TATTAGGATC TTTGTAGACA |
| 401 | CCGATGCTGA TGAAAGGATA CTACGCCGTA TGGTTCGAGA TGTTCAAGAA |
| 451 | CAAGGAGATA GCGTGGACTG CATCATGTCT CGTTATCTTT CTATGGTAAA |
| 501 | GCCTATGCAT GAGAAATTTA TAGAGCCGAC TCGGAAATAT GCTGATATCA |
| 551 | TTGTACATGG AAATTACCGA CAAAACGTAG TAACAAATAT TTTGTCACAG |
| 601 | AAAATTAAAA ATCATTTAGA GAATGCCCTG GAAAGCGATG AGACGTATTA |
| 651 | TATGGTCAAC TCTAAGTAA |

The PSORT algorithm predicts inner membrane (0.1022).

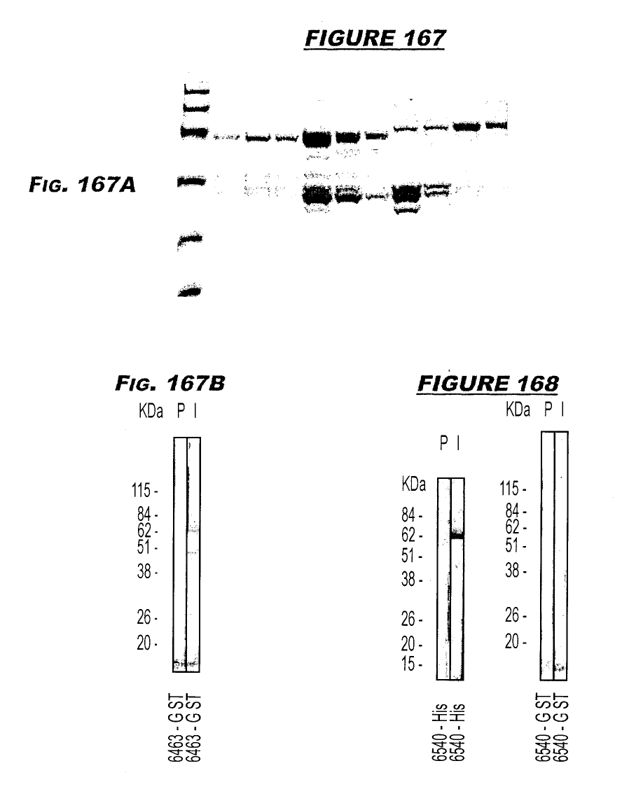

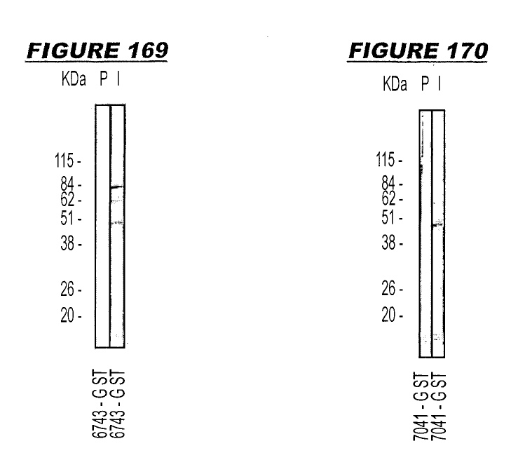

The proteins were expressed in E. coli and purified as his-tag products (FIG. 167A; 6463=lanes 2-4; 6540=lanes 5-7; 6743=lanes 8-9; 7041=lanes 10-11). The recombinant proteins were used to immunize mice, whose sera were used in Western blots (FIGS. 167B, 168, 169 & 170) and for FACS analysis.

These experiments show that cp6463, cp6540, cp6743 & cp7041 are surface-exposed and immunoaccessible proteins and that they are useful immunogens. These properties are not evident from the sequence alone.

### Example 171 and Example 172 and Example 173

The following C. pneumoniae protein (PID 4376632) was expressed <SEQ ID 341; cp6632>:

| | |
|---|---|
| 1 | VQLFQYMNES GWDWLCDFDS QGEGFQLSRL VGLLHSSWAL YEAKEQFYLP |
| 51 | EVSLLTWEEL IEMQLLSKPT KHGVAKDLCN VFEKHFQRFR QYLGSLDLNQ |
| 101 | RFENTFLNYP KYHLDRE* |

The cp6632 nucleotide sequence <SEQ ID 342> is:

| | |
|---|---|
| 1 | GTGCAATTAT TTCAATATAT GAATGAGTCC GGATGGGATT GGCTTTGTGA |
| 51 | TTTTGATTCT CAAGGCGAGG GATTCCAGTT ATCACGTCTG GTTGGGCTGT |
| 101 | TACATTCGTC CTGGGCATTA TACGAAGCAA AAGAGCAATT TTACCTTCCT |
| 151 | GAGGTTTCTC TATTGACCTG GGAAGAACTG ATAGAAATGC AGTTATTAAG |
| 201 | CAAACCAACA AAACACGGGG TTGCAAAAGA TCTTTGTAAT GTATTTGAAA |
| 251 | AACACTTTCA AAGGTTTAGA CAGTACCTAG GTTCCTTAGA TCTAAATCAA |
| 301 | AGGTTCGAAA ATACCTTCTT GAATTATCCT AAATACCATT TAGATAGGGA |
| 351 | GTGA |

The PSORT algorithm predicts cytoplasm (0.3627).

The following C. pneumoniae protein (PID 4376648) was also expressed <SEQ ID 343; cp6648>:

| | |
|---|---|
| 1 | MPVSSAPLPT SHRPSSGNLG LMEPNSKALK AKHQDKTTKT IKLLVKILVA |
| 51 | ILVIEVLGII AAFFIPGTPP ICLIILGGLI LTTVLCVLLL VIKLALVNKT |
| 101 | EGTTAEQQIK RKLSSKSIS* |

The cp6648 nucleotide sequence <SEQ ID 344> is:

| | |
|---|---|
| 1 | ATGCCCGTGT CCTCAGCCCC CCTACCCACA AGCCACCGCC CTTCCTCTGG |
| 51 | AAATCTAGGC CTCATGGAAC CAAATTCCAA AGCTCTAAAA GCAAAGCATC |
| 101 | AAGATAAAAC GACGAAGACG ATTAAACTTT TAGTTAAAAT CCTTGTTGCC |
| 151 | ATTCTAGTAA TAGAAGTTTT AGGAATAATT GCAGCTTTCT TTATTCCTGG |
| 201 | GACTCCTCCC ATCTGCTTGA TTATCCTAGG AGGCCTTATT CTTACAACAG |
| 251 | TACTCTGTGT GCTTCTTCTT GTTATAAAGC TTGCCCTTGT AAACAAAACC |
| 301 | GAAGGAACAA CTGCTGAACA GCAGATAAAA CGTAAACTCT CTTCTAAAAG |
| 351 | TATTTCTTAG |

The PSORT algorithm predicts inner membrane (0.6074).

The following C. pneumoniae protein (PID 4376497) was also expressed <SEQ ID 345; cp6497>:

| | |
|---|---|
| 1 | MKPNSIIFLE NTKHYPDIFR EGFVRDRHGL MEASDWLLST EITIIRSILG |
| 51 | AIPILGNILG AGRLYSVWYT SDEDWKKQVV * |

The cp6497 nucleotide sequence <SEQ ID 346> is:

| | |
|---|---|
| 1 | ATGAAGCCAA ATAGTATTAT TTTTTTAGAA AATACTAAGC ATTATCCCGA |
| 51 | CATCTTTCGA GAAGGATTTG TTCGTGATCG TCATGGACTA ATGGAAGCCT |
| 101 | CGGATTGGTT ACTTTCTACG GAAATTACGA TCATTCGCTC CATTCTGGGA |
| 151 | GCTATCCCTA TTTTAGGAAA TATTCTTGGA GCCGGACGAC TCTATAGCGT |
| 201 | TTGGTATACA AGTGACGAAG ATTGGAAAAA ACAAGTGGTT TGA |

The PSORT algorithm predicts inner membrane (0.145).

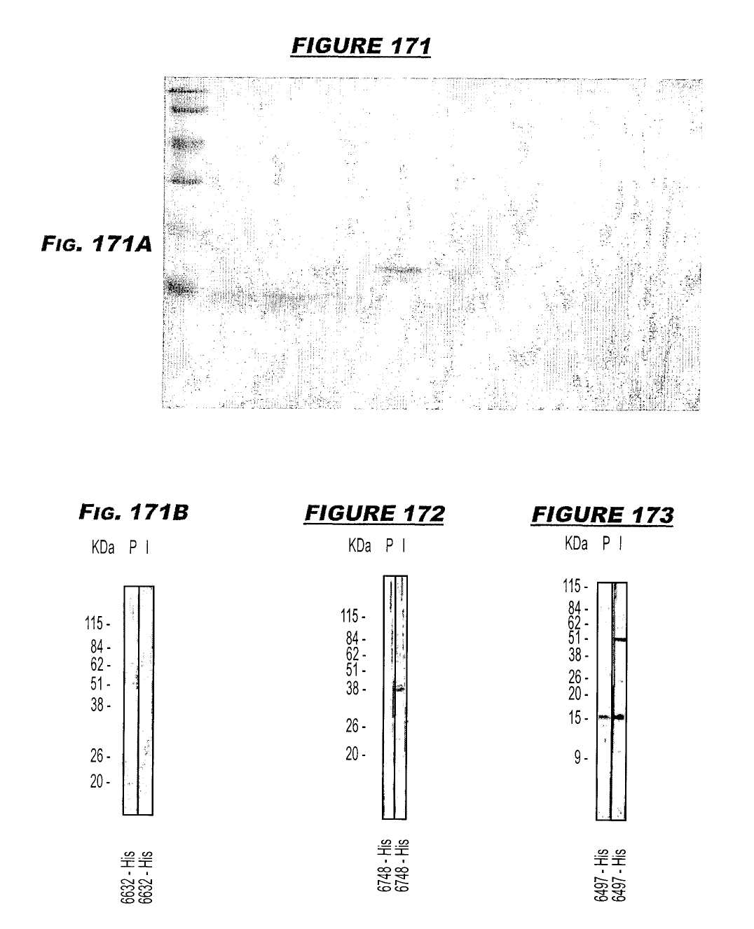

The proteins were expressed in E. coli and purified as his-tag products (FIG. 171A; 6632=lanes 5-7; 6648=lanes 8-10; 6497=lanes 2-4). The recombinant proteins were used to immunize mice, whose sera were used in Western blots (FIGS. 171B, 172, 173) and for FACS analysis.

These experiments show that cp6632, cp6648 and cp6497 are surface-exposed and immunoaccessible proteins and that they are useful immunogens. These properties are not evident from the sequence alone.

### Example 174, Example 175, Example 176, Example 177 and Example 178

The following C. pneumoniae protein (PID 4377200) was expressed <SEQ ID 347; cp7200>:

| | |
|---|---|
| 1 | MPVPIDNSSR NLQEVPESLE DLEQHAEESP THQSAESSSL QLSLASSAIS |
| 51 | SRVEQLSSLV LGMENSDFSS LRDVPIFSAI YESSTHTPVP TPLVGVGYIN |
| 101 | GSQSGYYDTQ RESLHLSQLL GSRRVEVVYN QGNFMEASLL NLCPRRPRRD |
| 151 | PSPISLALLE LWEAFFLEHP PGSTFNPIFF W* |

The cp7200 nucleotide sequence <SEQ ID 348> is:

| | |
|---|---|
| 1 | ATGCCCGTTC CTATAGATAA TTCCTCTCGC AACCTACAAG AAGTTCCAGA |
| 51 | AAGCCTAGAA GACCTCGAAC AACACGCAGA AGAATCTCCT ACTCATCAAA |
| 101 | GTGCAGAAAG CAGTTCTTTG CAACTGTCTC TAGCCTCCTC AGCAATTTCT |
| 151 | AGTAGAGTAG AACAACTATC TTCCCTCGTC TTAGGAATGG AAAATTCAGA |
| 201 | TTTCTCCTCT TTAAGAGACG TTCCTATCTT CTCAGCTATC TACGAATCTT |
| 251 | CAACACACAC ACCTGTCCCC ACTCCTCTAG TTGGCGTGGG ATATATCAAC |
| 301 | GGAAGTCAAT CAGGATACTA CGATACACAA AGAGAATCTC TTCACCTCAG |
| 351 | CCAATTGTTA GGAAGCCGAA GAGTTGAAGT TGTCTATAAC CAAGGAAACT |
| 401 | TCATGGAGGC CTCTTTGCTA AATCTGTGCC CCAGAAGACC TCGAAGAGAT |
| 451 | CCCTCTCCAA TTTCTTTAGC TCTATTAGAG CTCTGGGAAG CATTTTTTTT |
| 501 | AGAACACCCC CCAGGTAGCA CTTTTAATCC AATATTTTTT TGGTAA |

The PSORT algorithm predicts cytoplasm (0.3672).

The following C. pneumoniae protein (PID 4377235) was also expressed <SEQ ID 349; cp7235>:

| | |
|---|---|
| 1 | LNFVSTLTGS DFYAPVLEKL EEAFADTTGQ VILFSSSPDF IVHPIAQQLG |
| 51 | ISSWYASCYR DQSAEQTIYK KCLTGDKKAQ ILSYIKKINQ ARSHTFSDHI |
| 101 | LDLPFLMLGE EKTVVRPQGR LKKMAKKYYW NIV* |

The cp7235 nucleotide sequence <SEQ ID 350> is:

| | |
|---|---|
| 1 | TTGAATTTTG TATCGACTCT GACCGGCTCC GATTTTTATG CTCCTGTTTT |
| 51 | AGAAAAACTA GAAGAAGCTT TTGCAGATAC CACAGGACAG GTGATCCTTT |
| 101 | TTTCTTCTTC TCCAGACTTT ATTGTCCACC CCATAGCGCA GCAACTCGGG |
| 151 | ATTAGTTCTT GGTATGCGTC GTGTTATCGC GATCAGTCTG CAGAACAGAC |
| 201 | GATCTATAAA AAATGTCTTA CAGGGGATAA AAAAGCGCAA ATTTTGAGTT |
| 251 | ATATTAAAAA AATTAATCAA GCAAGAAGCC ATACCTTCTC CGACCATATT |
| 301 | TTAGATCTTC CTTTTCTTAT GCTGGGAGAA GAGAAAACCG TCGTTCGCCC |
| 351 | TCAGGGACGA CTCAAGAAAA TGGCAAAAAA ATATTACTGG AATATCGTTT |
| 401 | AA |

The PSORT algorithm predicts cytoplasm (0.3214).

The following C. pneumoniae protein (PID 4377268) was also expressed <SEQ ID 351; cp7268>:

| | |
|---|---|
| 1 | MMHRYFIPLL ALLIFSPSLV RAELQPSENR KGGWPTQLSC AEGSQLFCKF |
| 51 | EAAYNNAIEE GKPGILVFFS ERPTPEFADL TNGSFSLSTP IAKGFNVVVL |
| 101 | CPGLISPLDF FHKMDPVILY MGSFLEMFPE VEAVSGPRLC YILIDEQGGA |
| 151 | QCQAVLPLET KN* |

The cp7268 nucleotide sequence <SEQ ID 352> is:

| | |
|---|---|
| 1 | ATGATGCACC GTTATTTTAT TCCTTTATTA GCACTTCTCA TTTTCTCTCC |
| 51 | TTCTTTAGTC AGGGCAGAGC TACAACCAAG TGAAAACAGA AAAGGGGGGT |
| 101 | GGCCTACACA ACTTTCCTGT GCAGAAGGTT CGCAACTCTT CTGTAAATTC |
| 151 | GAAGCTGCCT ATAATAATGC AATTGAGGAA GGGAAACCTG GGATTTTAGT |
| 201 | CTTTTTCTCT GAGCGACCCA CACCAGAATT TGCCGACTTA ACGAATGGTT |
| 251 | CATTTTCTCT CTCTACGCCA ATCGCCAAGG GCTTTAATGT CGTTGTGTTA |
| 301 | TGCCCCGGGC TTATCAGTCC CTTAGACTTT TTCCACAAAA TGGATCCTGT |
| 351 | GATTCTCTAT ATGGGAAGTT TTCTAGAGAT GTTCCCTGAA GTGGAGGCAG |
| 401 | TTAGTGGCCC TCGCTTATGT TATATCTTAA TAGATGAACA GGGTGGGGCT |
| 451 | CAATGTCAGG CTGTCCTGCC TTTAGAAACA AAGAATTAG |

The PSORT algorithm predicts inner membrane (0.1235).

The following C. pneumoniae protein (PID 4377375) was also expressed <SEQ ID 353; cp7375>:

| | |
|---|---|
| 1 | MQRIIIVGID TGVGKTIVSA ILARALNAEY WKPIQAGNLE NSDSNIVHEL |
| 51 | SGAYCHPEAY RLHKPLSPHK AAQIDNVSIE ESHICAPKTT SNLIIETSGG |
| 101 | FLSPCTSKRL QGDVFSSWSC SWILVSQAYL GSINHTCLTV EAMRSRNLNI |
| 151 | LGMVVNGYPE DEEHWLTQEI KLPIIGTLAK EKEITKTIIS CYAEQWKEVW |
| 201 | TSNHQGIQGV SGTPSLNLH* |

The cp7375 nucleotide sequence <SEQ ID 354> is:

| | |
|---|---|
| 1 | ATGCAACGTA TCATCATTGT AGGAATCGAC ACTGGCGTAG GAAAAACCAT |
| 51 | TGTCAGTGCT ATCCTTGCTA GAGCACTTAA CGCAGAATAC TGGAAACCTA |
| 101 | TACAAGCAGG GAATCTAGAA AATTCAGATA GCAATATTGT TCATGAGCTA |
| 151 | TCGGGAGCCT ACTGTCATCC CGAAGCTTAT CGATTGCATA AGCCCTTGTC |
| 201 | TCCACACAAG GCAGCGCAAA TCGATAATGT AAGTATCGAA GAGAGTCATA |
| 251 | TTTGTGCGCC AAAAACAACT TCGAATCTGA TTATTGAGAC TTCAGGAGGA |
| 301 | TTTTTATCCC CCTGCACATC AAAAAGACTT CAGGGAGATG TGTTTTCTTC |
| 351 | TTGGTCATGT TCTTGGATTT TAGTGAGCCA AGCATATCTC GGAAGTATCA |
| 401 | ATCACACCTG TTTAACGGTA GAAGCAATGC GCTCACGAAA CCTCAATATC |
| 451 | TTAGGTATGG TGGTAAATGG GTATCCAGAG GACGAAGAGC ACTGGCTAAC |
| 501 | TCAAGAAATC AAGCTTCCTA TAATCGGGAC TCTTGCCAAG GAAAAAGAAA |
| 551 | TCACAAAGAC AATCATAAGC TGTTATGCCG AACAATGGAA GGAAGTATGG |
| 601 | ACAAGCAATC ATCAGGGAAT TCAGGGTGTA TCTGGCACCC CTTCACTCAA |
| 651 | TCTGCATTAG |

The PSORT algorithm predicts cytoplasm (0.0049).

The following C. pneumoniae protein (PID 4377388) was also expressed <SEQ ID 355; cp7388>:

| | |
|---|---|
| 1 | MQVLLSPQLP PPPQHSVGSI SSPSKLRVLA ITFLVFGMLL LISGALFLTL |
| 51 | GIPGLSAAIS FGLGIGLSAL GGVLMISGLL CLLVKREIPT VRPEEIPEGV |
| 101 | SLAPSEEPAL QAAQKTLAQL PKELDQLDTD IQEVFACLRK LKDSKYESRS |
| 151 | FLNDAKKELR VFDFVVEDTL SEIFELRQIV AQEGWDLNFL INGGRSLMMT |
| 201 | AESESLDLFH VSKRLGYLPS GDVRGEGLKK SAKEIVARLM SLHCEIHKVA |
| 251 | VAFDRNSYAM AEKAFAKALG ALEESVYRSL TQSYRDKFLE SERAKIPWNG |
| 301 | HITWLRDDAK SGCAEKKLRD AEERWKKFRK AVFWVEEDGG FDINNLLGDW |
| 351 | GTVLDPYRQE RMDEITFHEL YEKTTFLKRL HRKCALAKTT FEKKRSKKNL |
| 401 | QAVEEANARR LKYVRDWYDQ EFQKAGERLE KLHALYPEVS VSIRENKIQE |
| 451 | TRSNLEKAYE AIEENYRCCV REQEDYWKEE EKREAEFRER GNKILSPEEL |
| 501 | ESSLEQFDHG LKNFSEKLME LEGHILKLQK EATAEVENKI LSDAESRLEI |
| 551 | VFEDVKEMPC RIEEIEKTLR MAELPLLPTK KAFEKACSQY NSCAEMLEKV |
| 601 | KPYCKESLAY VTSKERLVSL DEDLRRAYTE CQKRFQGDSG LESEVRACRE |
| 651 | QLRERIQEFE TQGLDLVEKE LLCVSSRLRN TECDCVSGVK KEAPPGKKFY |
| 701 | AQYYDEIYRV RVQSRWMTMS ERLREGVQAC NKMLKAGLSE EDKVLKEEEY |
| 751 | WLYREERKNK EKRLVGTKIV ATQQRVAAFE SIEVPEIPEA PEEKPSLLDK |
| 801 | ARSLFTREDH T |

The cp7388 nucleotide sequence <SEQ ID 356> is:

| | |
|---|---|
| 1 | ATGCAAGTAC TTCTATCTCC GCAGCTACCC CCCCCCCCCC AACACTCTGT |
| 51 | AGGGTCGATT TCTTCTCCAT CTAAACTTCG CGTTTTAGCG ATTACTTTTT |
| 101 | TAGTTTTTGG TATGCTCTTA CTGATTTCAG GAGCTCTCTT TCTGACGTTA |
| 151 | GGGATTCCAG GATTGAGTGC AGCAATTTCT TTTGGATTAG GCATCGGTCT |
| 201 | CTCCGCATTA GGAGGAGTGC TGATGATTTC GGGACTACTA TGTCTTTTAG |
| 251 | TAAAACGAGA GATTCCGACA GTACGACCAG AAGAAATTCC TGAAGGGGTT |
| 301 | TCGCTGGCTC CTTCTGAGGA GCCAGCTCTA CAGGCAGCTC AGAAGACTTT |
| 351 | AGCTCAGCTG CCTAAGGAAT TGGATCAGTT AGATACAGAT ATTCAGGAAG |
| 401 | TGTTCGCATG TTTAAGAAAG CTGAAAGATT CTAAGTATGA AAGTCGAAGT |
| 451 | TTTTTAAACG ATGCTAAGAA GGAGCTTCGA GTTTTTGACT TTGTGGTTGA |
| 501 | GGATACCCTC TCGGAGATTT TCGAGTTGCG GCAGATTGTG GCTCAAGAGG |
| 551 | GATGGGATTT AAACTTTTTG ATCAATGGGG GACGAAGCCT CATGATGACT |
| 601 | GCAGAATCTG AATCGCTTGA TTTGTTTCAT GTATCGAAGC GGCTAGGGTA |
| 651 | TTTACCTTCT GGGGATGTTC GAGGGGAGGG GTTAAAGAAA TCTGCGAAGG |
| 701 | AGATAGTCGC TCGTTTGATG AGCTTGCATT GCGAGATTCA CAAGGTGGCG |
| 751 | GTAGCGTTTG ATAGGAATTC CTATGCGATG GCAGAAAAGG CGTTTGCGAA |
| 801 | AGCGTTGGGA GCTTTAGAAG AGAGTGTGTA TCGGAGTCTG ACGCAGAGTT |
| 851 | ATAGAGATAA ATTTTTGGAG AGCGAGAGGG CGAAGATCCC ATGGAATGGG |
| 901 | CATATAACCT GGTTAAGAGA TGATGCGAAG AGTGGGTGTG CTGAAAAGAA |
| 951 | GCTTCGGGAT GCCGAGGAAC GTTGGAAGAA ATTTAGGAAA GCAGTCTTTT |
| 1001 | GGGTAGAAGA AGACGGGGGC TTTGACATCA ATAATCTCCT TGGAGACTGG |
| 1051 | GGGACAGTGC TTGATCCTTA TAGACAAGAG AGAATGGACG AGATAACGTT |
| 1101 | CCATGAGTTG TATGAAAAAA CTACGTTTTT GAAAAGACTG CACAGAAAGT |
| 1151 | GTGCGTTAGC GAAAACAACC TTTGAAAAGA AGAGATCTAA AAAGAATTTG |
| 1201 | CAGGCAGTCG AGGAGGCGAA TGCACGTAGG TTGAAATATG TAAGGGATTG |
| 1251 | GTATGATCAG GAGTTTCAGA AAGCAGGGGA GAGATTAGAG AAACTGCATG |
| 1301 | CTTTGTATCC TGAGGTTTCA GTCTCTATAA GAGAGAACAA AATACAAGAG |
| 1351 | ACGCGCTCTA ATTTAGAGAA AGCCTATGAG GCTATCGAAG AGAACTATCG |
| 1401 | TTGCTGTGTC CGAGAGCAAG AGGACTACTG GAAAGAAGAA GAGAAAAGGG |
| 1451 | AAGCGGAGTT TAGGGAGAGG GGAAACAAGA TTCTTTCTCC TGAGGAGCTG |
| 1501 | GAAAGTTCTT TGGAGCAATT CGACCATGGT TTGAAAAATT TTTCTGAGAA |
| 1551 | ATTAATGGAA TTGGAAGGGC ATATCTTAAA ACTTCAGAAA GAAGCCACAG |
| 1601 | CAGAGGTGGA GAATAAAATA CTTTCAGATG CAGAGAGCCG CCTTGAGATT |
| 1651 | GTATTTGAAG ATGTCAAGGA GATGCCCTGT CGAATTGAGG AGATAGAGAA |
| 1701 | GACGCTGCGT ATGGCGGAGC TGCCCCTACT TCCTACGAAG AAGGCGTTTG |
| 1751 | AGAAGGCCTG CTCACAATAT AATAGCTGCG CAGAGATGTT GGAGAAGGTG |
| 1801 | AAGCCTTACT GCAAGGAGAG CCTCGCCTAT GTGACTAGCA AAGAGCGTTT |
| 1851 | AGTGAGCTTG GATGAAGATT TACGACGAGC CTACACAGAG TGTCAGAAGA |
| 1901 | GATTCCAGGG GGATTCGGGT TTGGAGTCGG AAGTAAGAGC CTGTCGAGAG |
| 1951 | CAACTGCGAG AGCGGATCCA AGAGTTTGAA ACTCAAGGGC TGGACTTGGT |
| 2001 | GGAAAAAGAG TTGCTTTGTG TGAGTAGTAG ATTAAGAAAT ACAGAGTGCG |
| 2051 | ATTGTGTATC TGGTGTTAAG AAAGAAGCAC CTCCTGGTAA GAAGTTTTAT |
| 2101 | GCCCAGTATT ATGATGAGAT TTATCGAGTT AGAGTTCAAT CCCGATGGAT |
| 2151 | GACGATGTCT GAGAGATTGA GAGAGGGAGT TCAAGCATGC AACAAGATGT |
| 2201 | TGAAGGCAGG CCTAAGCGAA GAAGATAAGG TTCTTAAAGA AGAAGAGTAT |
| 2251 | TGGTTGTATC GAGAGGAGAG AAAGAATAAA GAGAAACGTT TGGTTGGTAC |
| 2301 | TAAGATAGTA GCAACGCAGC AGCGAGTTGC AGCATTTGAA TCCATAGAAG |
| 2351 | TTCCTGAGAT TCCTGAGGCC CCAGAGGAGA AACCGAGTTT GCTGGATAAA |
| 2401 | GCGCGTTCTT TATTTACTCG CGAGGACCAT ACCTAG |

The PSORT algorithm predicts inner membrane (0.461).

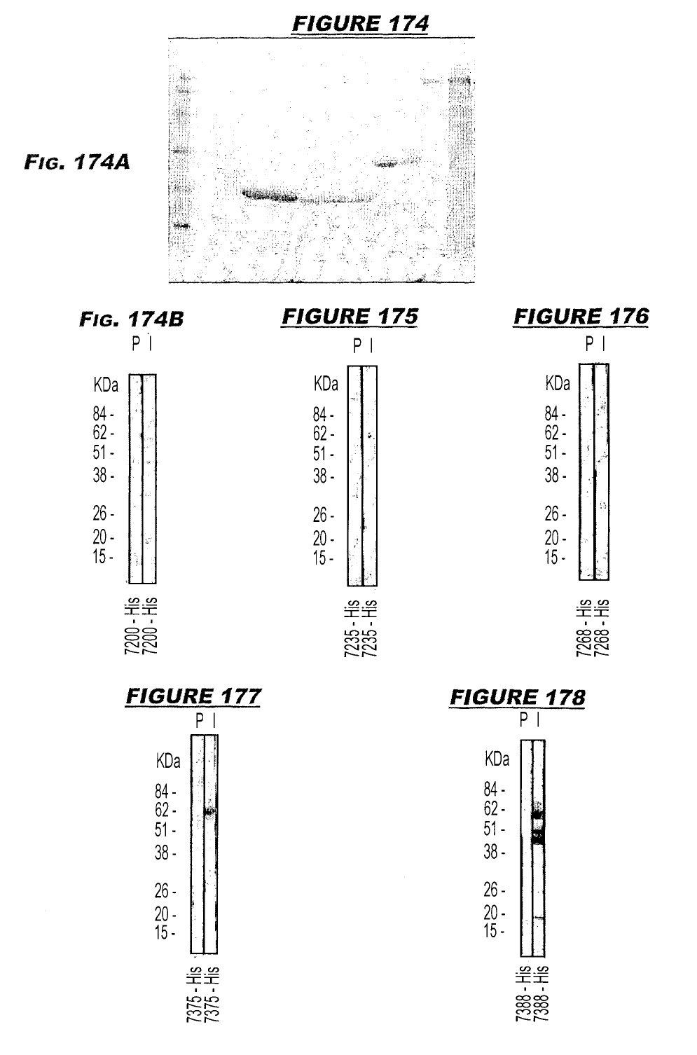

The proteins were expressed in E. coli and purified as his-tag products (FIG. 174: 7200=lanes 2-3; 7236=lanes 4-5; 7268=lanes 6-8; 7375=lanes 9-10; 7388=lanes 11-12). The recombinant proteins were used to immunize mice, whose sera were used in Western blots (FIGS. 174, 175, 176, 177 & 178) and for FACS analysis.

These experiments show that cp7200, cp7235, cp7268, cp7375 & cp7388 are surface-exposed and immunoaccessible proteins and that they are useful immunogens. These properties are not evident from the sequence alone.

### Example 179

The following C. pneumoniae protein (PID 4376723) was expressed <SEQ ID 357; cp6723>:

| | |
|---|---|
| 1 | MATSVAPSPV PESSPLSHAT EVLNLPNAYI TQPHPIPAAP WETFRSKLST |
| 51 | KHTLCFALTL LLTLGGTISA GYAGYTGNWI ICGIGLGIIV LTLTLALLLA |
| 101 | IPLKNKQTGT KLIDEISQDI SSIGSGFVQR YGLMFSTIKS VHLPELTTQN |
| 151 | QEKTRILNEI EAKKESIQNL ELKITECQNK LAQKQPKRKS SQKSFMRSIK |
| 201 | HLSKNPVILF DC* |

The cp6723 nucleotide sequence <SEQ ID 358> is:

| | |
|---|---|
| 1 | ATGGCAACTT CCGTAGCCCC ATCACCAGTC CCCGAGAGCA GCCCTCTCTC |
| 51 | TCATGCTACA GAAGTTCTCA ATCTTCCTAA TGCTTATATT ACGCAGCCTC |
| 101 | ATCCGATTCC AGCGGCTCCT TGGGAGACCT TTCGCTCCAA ACTTTCCACA |
| 151 | AAGCATACGC TCTGTTTTGC CTTAACACTA CTGTTAACCT TAGGGGGAAC |
| 201 | GATCTCAGCA GGTTACGCAG GATATACTGG AAACTGGATC ATCTGTGGCA |
| 251 | TCGGCTTGGG AATTATCGTA CTCACACTGA TTCTTGCTCT TCTTCTAGCA |
| 301 | ATCCCTCTTA AAAATAAGCA GACAGGAACA AAACTGATTG ATGAGATATC |
| 351 | TCAAGACATT TCCTCTATAG GATCAGGATT TGTTCAGAGA TACGGGTTGA |
| 401 | TGTTCTCTAC AATTAAAAGC GTGCATCTTC CAGAGCTGAC AACACAAAAT |
| 451 | CAAGAAAAAA CAAGAATTTT AAATGAAATT GAAGCGAAAA AGGAATCGAT |
| 501 | CCAAAATCTT GAGCTTAAAA TTACTGAGTG CCAAAACAAG TTAGCACAGA |
| 551 | AACAGCCGAA ACGGAAATCA TCTCAGAAAT CATTTATGCG TAGTATTAAG |
| 601 | CACCTCTCCA AGAACCCTGT AATTTTGTTC GATTGCTGA |

The PSORT algorithm predicts inner membrane (0.6095).

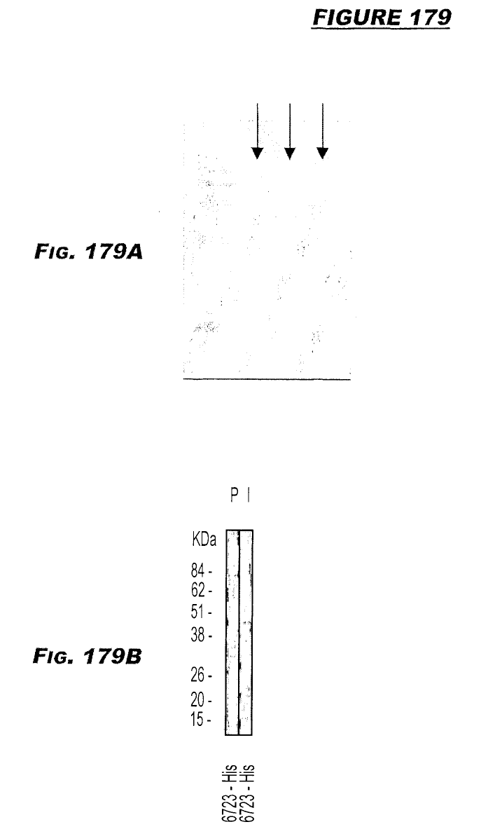

The protein was expressed in E. coli and purified as a his-tag product (FIG. 179A). The recombinant protein was used to immunize mice, whose sera were used in a Western blot (FIG. 179B) and for FACS analysis.

These experiments show that cp6723 is a surface-exposed and immunoaccessible protein, and that it is a useful immunogen. These properties are not evident from the sequence alone.

### Example 180

The following C. pneumoniae protein (PID 4376749) was expressed <SEQ ID 359; cp6749>:

| | |
|---|---|
| 1 | MSYYFSLWYL KVQQHFQAAF DFTRSLCSRI SNFALGVIAL LPIIGQLYVG |
| 51 | LDWLLSRIKK PEFPSDVDQI VRVEHVVGHD HRSRVEDILK RQRLSLEPRD |
| 101 | EGKVHGDLPS APFF* |

The cp6749 nucleotide sequence <SEQ ID 360> is:

| | |
|---|---|
| 1 | ATGAGTTATT ACTTTTCTCT TTGGTATCTG AAGGTGCAAC AGCACTTTCA |
| 51 | AGCAGCATTT GATTTTACTC GCTCCCTGTG TTCACGAATT TCTAATTTTG |
| 101 | CTTTGGGAGT GATTGCATTG CTTCCTATTA TTGGGCAGTT GTATGTAGGG |
| 151 | CTGGACTGGC TCCTCTCTAG GATAAAAAAG CCAGAATTTC CTTCCGATGT |
| 201 | GGATCAGATC GTGCGAGTAG AACACGTCGT GGGTCACGAC CATAGAAGTC |
| 251 | GAGTTGAAGA TATTCTAAAG AGACAAAGGC TCTCATTAGA GCCTAGAGAC |
| 301 | GAGGGGAAGG TTCACGGAGA TCTGCCTTCA GCTCCTTTTT TTTGA |

The PSORT algorithm predicts inner membrane (0.2996).

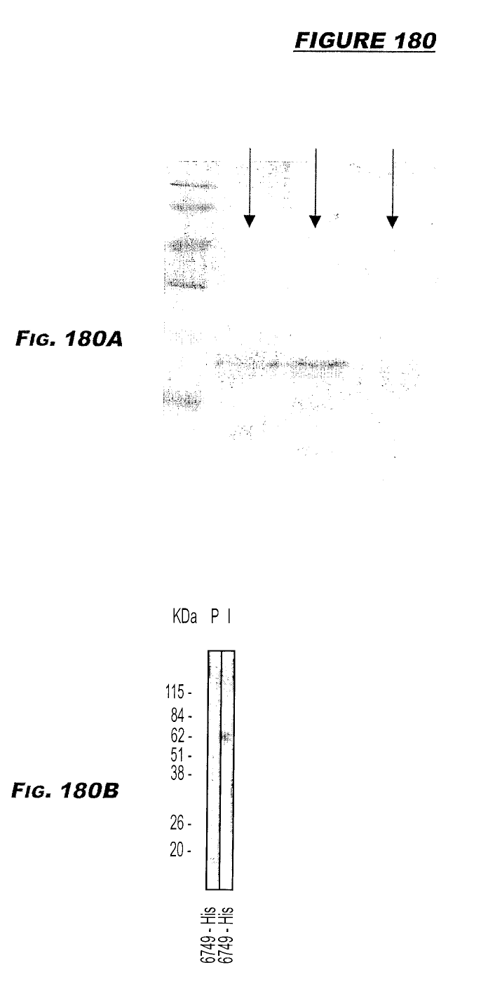

The protein was expressed in E. coli and purified as a his-tag product (FIG. 180A). The recombinant protein was used to immunize mice, whose sera were used in a Western blot (FIG. 180B) and for FACS analysis.

These experiments show that cp6749 is a surface-exposed and immunoaccessible protein, and that it is a useful immunogen. These properties are not evident from the sequence alone.

### Example 181, Example 182, Example 183, Example 184 and Example 185

The following C. pneumoniae protein (PID 4376301) was expressed <SEQ ID 361; cp6301>:

| | |
|---|---|
| 1 | LNQDLQNVYQ ECQKATGLES EVSAYRDHLR EQITEFETQG LDVIKEELLF |
| 51 | VSSTLKSKLS YDPLIADIPC MKFYEEYYDG IDKARVQSRW LEKSERYRKA |
| 101 | KKGFQEMLKE GLFKEDQALK KAEYRLLREK RMNKEKLLIC NKIEAAQQRV |
| 151 | QEFGPSDS* |

The cp6301 nucleotide sequence <SEQ ID 362> is:

| | |
|---|---|
| 1 | TTGAATCAGG ATTTACAAAA TGTATACCAA GAGTGCCAGA AGGCTACAGG |
| 51 | TTTAGAATCG GAAGTGAGTG CATATAGAGA TCATCTTAGA GAGCAGATCA |
| 101 | CAGAGTTTGA AACTCAAGGG CTGGACGTGA TAAAAGAAGA ACTTCTTTTT |
| 151 | GTGAGTAGTA CTCTCAAAAG TAAATTGAGC TATGATCCAT TAATAGCAGA |
| 201 | CATTCCCTGT ATGAAGTTTT ATGAGGAGTA TTATGATGGC ATTGATAAAG |
| 251 | CGAGAGTTCA ATCCCGATGG CTGGAGAAGT CTGAGAGGTA TAGAAAGGCG |
| 301 | AAGAAGGGAT TCCAAGAGAT GCTGAAGGAA GGCCTATTCA AAGAAGATCA |
| 351 | GGCTTTGAAA AAAGCAGAGT ATAGATTACT TCGAGAGAAG AGAATGAATA |
| 401 | AGGAGAAGCT TTTGATTTGC AATAAGATAG AAGCAGCTCA GCAGCGAGTC |
| 451 | CAAGAATTTG GACCCTCGGA TTCATAA |

The PSORT algorithm predicts cytoplasm (0.4621).

The following C. pneumoniae protein (PID 4376558) was also expressed <SEQ ID 363; cp6558>:

| | |
|---|---|
| 1 | MNIPAPQVPV IDEPVVNNTS SYGLSLKSSL RPITYLILAI LAIATLMSVL |
| 51 | YFCGIISVGT FVLGMLIPLS VCSVLCVAYL FYQQSSIEKT KVFSITSPSV |
| 101 | FFSDEDLNLL LGREEDSVSA IDELLKNFPA DDFRRPKMLP YSNFLDEQGR |
| 151 | PNESREEDSH TSKIL* |

The cp6558 nucleotide sequence <SEQ ID 364> is:

| | |
|---|---|
| 1 | ATGAACATAC CCGCTCCCCA AGTACCAGTC ATAGATGAGC CTGTAGTGAA |
| 51 | CAACACAAGT AGCTATGGTC TTTCATTGAA AAGTAGTTTA AGACCGATTA |
| 101 | CTTATTTGAT TTTAGCTATC TTAGCTATAG CCACACTGAT GTCTGTTCTC |
| 151 | TACTTTTGTG GCATCATTAG TGTTGGGACG TTTGTTTTGG GCATGCTGAT |
| 201 | CCCTCTATCG GTCTGCTCTG TTCTTTGCGT TGCCTATTTA TTCTATCAGC |
| 251 | AATCTTCTAT AGAAAAGACT AAGGTCTTTT CTATAACCAG TCCTTCAGTA |
| 301 | TTTTTCTCTG ATGAGGATCT TAATTTACTC TTAGGTCGAG AAGAAGATTC |
| 351 | AGTGTCTGCA ATTGATGAAC TTCTTAAGAA CTTTCCAGCT GATGATTTCC |
| 401 | GTAGGCCGAA GATGCTTCCT TATTCAAATT TTCTAGATGA GCAGGGAAGG |
| 451 | CCTAATGAGA GTAGGGAAGA AGACTCTCAT ACTTCCAAGA TCTTATAA |

The PSORT algorithm predicts inner membrane (0.4630).

The following C. pneumoniae protein (PID 4376630) was also expressed <SEQ ID 365; cp6630>:

| | |
|---|---|
| 1 | MSMTIVPHAL FKNHCECHST FPLSSRTIVR IAIASLFCIG ALAALGCLAP |
| 51 | PVSYIVGSVL AFIAFVILSL VILALIFGEK KLPPTPRIIP DRFTHVIDEA |
| 101 | YGLSISAFVR EQQVTLAEFR QFSTALLCNI SPEEKIKQLP SELRSKVESF |
| 151 | GISRLAGDLE KNNWPIFEDL LSQTCPLYWL QKFISAGDPQ VCRDLGVPRE |
| 201 | CYGYYWLGPL GYSTAKATIF CKETHHILQQ LTKEDVLLLK NKALQEKWDT |
| 251 | DEVKAIVERI YTTYTARGTL KTEAGGLTKE TISKELLLLS LHGYSFDQLQ |
| 301 | LITQLPRDAW DWLCFVDNST AYNLQLCALV GALSSQNLLD ESSIDFDVNL |
| 351 | GLYVIQDLKE AVQAFSASDE PKKELGKFLL RHLSSVSKRL ESVLRQGLHR |
| 401 | IALEHGNARA RVYDVNFVTG ARIHRKTSIF FKD* |

The cp6630 nucleotide sequence <SEQ ID 366> is:

| | |
|---|---|
| 1 | ATGAGCATGA CGATCGTTCC ACATGCTTTA TTTAAAAATC ATTGCGAGTG |
| 51 | TCATTCTACC TTTCCTTTGA GTTCAAGGAC TATTGTAAGA ATAGCCATTG |
| 101 | CCAGCCTCTT TTGTATAGGT GCATTAGCAG CTTTAGGCTG TTTGGCTCCT |
| 151 | CCCGTTTCTT ATATTGTTGG GAGTGTTTTA GCTTTTATTG CCTTTGTCAT |
| 201 | TCTTTCTTTA GTAATTTTAG CTTTGATTTT TGGAGAGAAG AAGCTTCCAC |
| 251 | CAACACCAAG AATCATTCCT GATAGATTTA CTCACGTGAT AGATGAAGCT |
| 301 | TATGGCCTTT CAATCTCTGC ATTTGTAAGA GAACAGCAGG TAACATTAGC |
| 351 | CGAGTTTAGA CAATTTTCTA CTGCCCTGTT GTGTAACATA TCTCCTGAAG |
| 401 | AGAAAATCAA ACAATTGCCT TCTGAATTGC GAAGTAAAGT AGAGAGTTTT |
| 451 | GGTATTAGCA GGCTCGCAGG TGATTTAGAA AAGAATAATT GGCCAATATT |
| 501 | TGAAGATCTT TTAAGCCAAA CCTGCCCGTT ATATTGGCTT CAGAAATTTA |
| 551 | TATCAGCAGG AGATCCACAA GTTTGTAGAG ACCTAGGTGT CCCTAGAGAA |
| 601 | TGTTATGGGT ACTATTGGCT AGGGCCTTTG GGATACAGTA CAGCTAAGGC |
| 651 | TACAATTTTT TGTAAAGAGA CGCATCATAT TCTTCAACAA TTAACGAAAG |
| 701 | AGGACGTTCT TTTATTAAAA AACAAGGCTC TTCAAGAGAA ATGGGATACT |
| 751 | GATGAAGTCA AAGCAATTGT AGAGCGTATC TACACTACCT ATACGGCACG |
| 801 | AGGAACTCTA AAGACCGAAG CAGGGGGACT TACAAAAGAG ACAATCAGTA |
| 851 | AGGAATTGCT ATTGTTGAGC TTGCATGGCT ATTCTTTTGA TCAGCTACAG |
| 901 | CTGATCACTC AACTTCCTAG AGATGCTTGG GATTGGCTGT GTTTTGTAGA |
| 951 | TAACAGTACC GCATACAACC TTCAGCTTTG TGCTCTTGTA GGAGCTTTGT |
| 1001 | CATCCCAAAA TCTTCTTGAC GAATCTTCTA TCGATTTTGA TGTAAACCTA |
| 1051 | GGCCTGTATG TGATTCAGGA TCTAAAAGAA GCTGTTCAAG CATTTTCTGC |
| 1101 | TTCTGATGAG CCAAAGAAAG AACTAGGTAA ATTCTTGTTA AGGCATTTGA |
| 1151 | GTTCAGTTTC TAAGCGATTA GAGAGTGTAT TAAGACAGGG TCTTCACAGA |
| 1201 | ATAGCTCTAG AGCATGGAAA TGCCAGAGCT AGGGTTTATG ACGTCAATTT |
| 1251 | TGTAACAGGA GCTAGAATTC ATAGGAAGAC GAGTATCTTC TTTAAAGACT |
| 1301 | AA |

The PSORT algorithm predicts inner membrane (0.7092).

The following C. pneumoniae protein (PID 4376633) was also expressed <SEQ ID 367; cp6633>:

| | |
|---|---|
| 1 | MVNIQPVYRN TQVNYSQATQ FSVCQPALSL IIVSVVAAVL AIVALVCSQS |
| 51 | LLSIELGTAL VLVSLILFAS AMFMIYKMRQ EPKELLIPKK IMELIQEHYP |
| 101 | SIVVDFIRDQ EVSIYEIHHL ISILNKTNVF DKAPVYLQEK LLQFGIEKFK |
| 151 | DVHPSKLPNF EEILLQHCPL HWLGRLVYPM VSDVTPGTYG YYWCGPLGLY |
| 201 | ENAPSLFERR SLLLLKKISF GEFALLEDGL KKNTWSSSEL VQIRQNLFTR |
| 251 | YYADKEEVDE AELNADYEQF DSLLHLIFSH KLS* |

The cp6633 nucleotide sequence <SEQ ID 368> is:

```
    1 ATGGTTAATA TACAGCCTGT GTATAGGAAT ACCCAAGTCA ACTATAGTCA
  51 GGCTACCCAA TTTTCGGTGT GCCAGCCAGC GCTTAGCCTG ATTATCGTTT
101 CTGTTGTTGC TGCTGTACTC GCTATTGTAG CTTTGGTATG CAGTCAATCT
151 CTTTTATCCA TAGAGTTAGG AACTGCTCTT GTTCTAGTTT CTCTTATTCT
201 TTTTGCTTCT GCTATGTTTA TGATTTATAA GATGAGACAA GAACCTAAGG
251 AGTTGCTGAT CCCTAAGAAA ATCATGGAAC TCATCCAAGA ACATTATCCA
301 AGTATTGTTG TTGATTTTAT TAGAGATCAG GAGGTTTCCA TTTATGAGAT
351 ACATCACTTG ATCTCTATTC TTAATAAGAC GAATGTTTTC GACAAAGCAC
401 CAGTATATTT ACAAGAAAAA CTCTTACAGT TTGGCATTGA GAAGTTCAAA
451 GATGTACATC CAAGTAAGCT CCCTAATTTT GAAGAAATTC TTCTACAGCA
501 TTGCCCATTG CATTGGTTGG GACGTCTGGT ATATCCCATG GTATCGGATG
551 TCACTCCAGG AACCTATGGA TACTATTGGT GTGGTCCTTT AGGACTGTAC
601 GAGAACGCTC CCTCTCTTTT TGAACGTCGA TCTCTTCTAT TGTTAAAGAA
651 AATTAGCTTT GGAGAGTTTG CTCTTTTAGA AGATGGTCTC AAGAAAAACA
701 CGTGGAGTTC TTCGGAACTC GTTCAAATCA GACAAAACCT TTTTACAAGA
751 TATTATGCTG ATAAAGAAGA GGTAGATGAA GCAGAGTTAA ACGCTGATTA
801 CGAACAGTTT GATTCCCTCC TTCACCTTAT TTTTTCTCAC AAGCTCTCTT
851 GA
```

The PSORT algorithm predicts inner membrane (0.7283).

The following C. pneumoniae protein (PID 4376642) was also expressed <SEQ ID 369; cp6642>:

```
    1 MATTSPISLT VDHPLVDTKK KSCSNFDKIQ SRILLITAIF AVLVTIGTLL
  51 IGLLLNIPVI YFLTGISFIA VVLSNFILYK RATTLLKPRA CGKHKEIKPK
101 RVSTNLQYSS ISIAINRSKE NWEHQPKDLQ NLPAPSALLT DNPYEIWKAK
151 HSLFSLVSLL PGGNPEHLLI SASENLGKTL LIEETSQNAP ISSYVDTTPS
201 PKSLLNEAIQ ETRVEINTEL PAGDSGERLY WQPDFRGRVF LPQIPTTPEA
251 IYQYYYALYV TYIQTAINTN TQIIQIPLYS LREHLYSREL PPQSRMQQSL
301 AMITAVKYMA ELHPEYPLTI ACVERSLAQL PQESIEDLS*
```

The cp6642 nucleotide sequence <SEQ ID 370> is:

```
      1 ATGGCTACAA TCTCACCCAT ATCTTTAACT GTAGATCATC CCCTAGTAGA
    51 CACTAAAAAA AAATCCTGCA GCAACTTTGA TAAGATTCAG TCTCGAATTC
  101 TATTGATTAC TGCAATCTTT GCTGTCTTAG TTACTATAGG GACCCTACTT
  151 ATTGGTTTGC TTTTAAATAT TCCTGTTATC TATTTCCTCA CAGGAATTTC
  201 ATTTATTGCT GTTGTTCTTA GCAACTTTAT CCTTTATAAA CGAGCAACCA
  251 CCCTCTTAAA ACCGCGTGCT TGTGGCAAAC ACAAAGAAAT AAAACCAAAA
  301 AGGGTCTCCA CCAACCTACA GTATTCTTCT ATCTCTATCG CAATCAATCG
  351 TTCTAAAGAA AACTGGGAAC ACCAACCCAA GGACCTACAG AATCTCCCCG
  401 CACCCTCTGC ATTACTCACA GATAACCCTT ACGAGATATG GAAAGCTAAA
  451 CATTCACTGT TTTCCCTAGT ATCCCTCCTA CCGGGAGGCA ATCCAGAACA
  501 TCTCTTAATT TCAGCTTCCG AAAATTTAGG AAAGACTCTG TTAATTGAAG
  551 AAACCTCGCA AAATGCGCCT ATATCCTCCT ACGTAGATAC CACTCCCTCC
  601 CCAAAATCCT TGCTCAATGA GGCAATTCAG GAAACCAGGG TAGAAATAAA
  651 TACAGAACTC CCTGCGGGAG ATTCAGGAGA ACGTTTATAC TGGCAACCCG
  701 ATTTCCGAGG CCGCGTCTTC CTCCCACAAA TACCAACAAC TCCTGAAGCC
  751 ATCTACCAAT ACTACTATGC ACTCTATGTC ACTTATATCC AGACTGCGAT
  801 CAATACGAAC ACCCAAATTA TCCAAATCCC TTTATACAGC TTGAGGGAGC
  851 ATCTCTATTC TAGAGAATTG CCCCCGCAAT CAAGAATGCA ACAATCTTTG
  901 GCTATGATTA CAGCAGTAAA ATACATGGCC GAGCTGCACC CAGAATATCC
  951 GCTAACTATT GCTTGTGTTG AAAGATCCTT AGCCCAACTA CCTCAAGAAA
1001 GTATTGAGGA TCTCTCTTAG
```

The PSORT algorithm predicts inner membrane (0.5288).

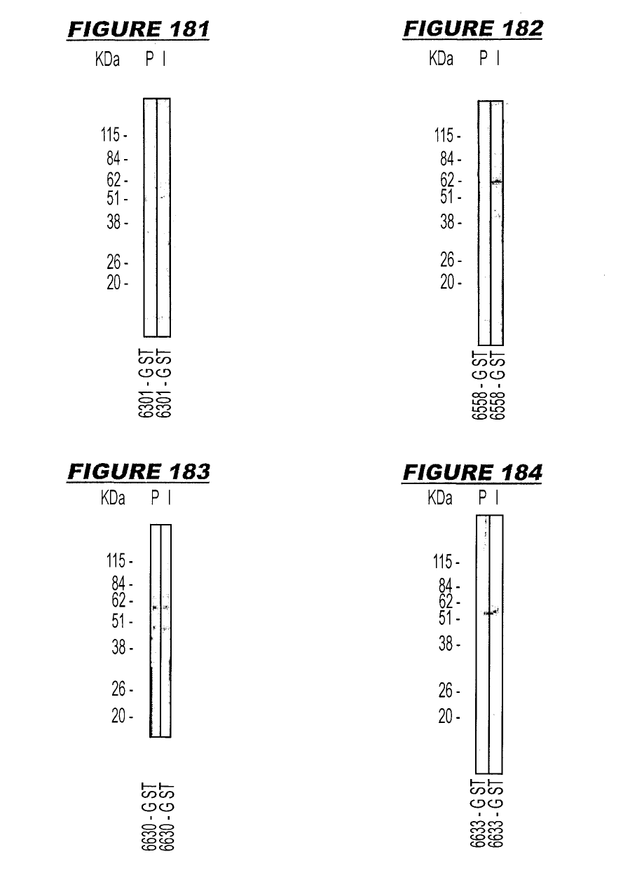

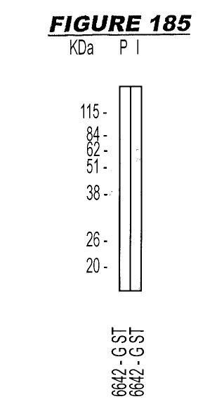

The proteins were expressed in E. coli and purified as GST-fusion products. The recombinant proteins were used to immunize mice, whose sera were used in Western blots (FIGS. 181-185) and for FACS analysis.

These experiments show that cp6301, cp6558, cp6630, cp6633 and cp6642 are surface-exposed and immunoaccessible proteins, and that they are useful immunogens. These properties are not evident from their sequences alone.

### Example 186

The following C. pneumoniae protein (PID 4376389) was expressed <SEQ ID 371; cp6389>:

```
    1 MSEVKPLFLK NDSFDLATQR FQNLINMLQE QAEIYNEYEE KNARVQNEIK
  51 EQKDFVKRCI EDFEARGLGV LKEELASLTR DFHDKAKAET SMLIECPCIG
101 FYYSIHQEEQ RQRQERLQKM AERYRDCKQV LEAVQVEQKD MISSRVVVDD
151 SYFEEEKEEQ KVDNRKKEQD *
```

The cp6389 nucleotide sequence <SEQ ID 372> is:

```
    1 ATGTCAGAAG TGAAGCCTTT GTTTTTAAAG AATGACTCTT TTGATTTGGC
  51 AACTCAGAGA TTCCAGAATC TAATTAACAT GCTACAAGAG CAAGCCGAGA
101 TATATAACGA GTATGAAGAA AAGAATGCTA GGGTTCAGAA TGAGATTAAG
151 GAGCAAAAGG ACTTTGTGAA AAGATGCATA GAGGACTTTG AAGCCAGAGG
201 ACTGGGGGTG CTAAAAGAAG AGCTTGCATC TTTGACGCGT GATTTCCATG
251 ATAAAGCAAA AGCAGAGACT TCTATGCTCA TTGAATGTCC TTGTATTGGT
301 TTTTATTATA GTATTCATCA GGAGGAACAA AGGCAAAGGC AAGAAAGGCT
351 TCAAAAGATG GCTGAGCGCT ATAGGGACTG TAAACAAGTC TTGGAGGCTG
401 TCCAGGTGGA GCAAAAAGAT ATGATATCTT CTAGAGTCGT TGTCGATGAC
451 AGCTACTTTG AAGAAGAAAA AGAAGAACAA AAGGTGGATA ACAGAAAGAA
501 AGAACAGGAC TAG
```

The PSORT algorithm predicts cytoplasm (0.3193).

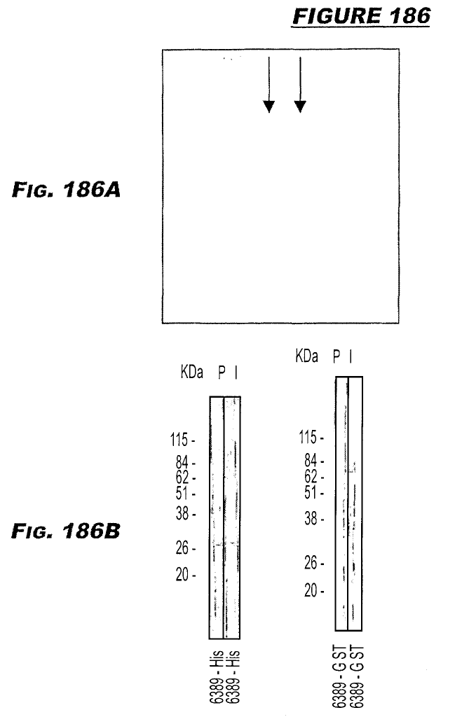

The protein was expressed in E. coli and purified as a GST-fusion product (FIG. 186A) and also in his-tagged form. The recombinant proteins were used to immunize mice, whose sera were used in a Western blot (FIG. 186B) and for FACS analysis.

These experiments show that cp6389 is a surface-exposed and immunoaccessible protein, and that it is a useful immunogen. These properties are not evident from the sequence alone.

### Example 187

The following C. pneumoniae protein (PID 4376792) was expressed <SEQ ID 373; cp6792>:

```
    1 VLQEHFFLSE DVITLAQQLL GHKLITTHEG LITSGYIVET EAYRGPDDKA
  51 CHAYNYRKTQ RNRAMYLKGG SAYLYRCYGM HHLLNVVTGP EDIPHAVLIR
101 AILPDQGKEL MIQRRQWRDK PPHLLTNGPG KVCQALGISL ENNRQRLNTP
151 ALYISKEKIS GTLTATARIG IDYAQEYRDV PWRFLLSPED SGKVLS*
```

The cp6792 nucleotide sequence <SEQ ID 374> is:

```
    1 GTGCTACAAG AACATTTTTT TCTATCGGAA GATGTAATTA CACTAGCGCA
  51 ACAGCTTTTA GGACATAAAC TCATCACAAC ACATGAGGGT CTGATAACTT
101 CAGGTTACAT TGTAGAAACC GAAGCGTATC GTGGCCCTGA TGACAAAGCA
151 TGCCACGCCT ACAACTACAG AAAAACTCAG AGGAACAGAG CGATGTACCT
201 GAAAGGAGGC TCTGCTTACC TCTACCGTTG CTATGGCATG CATCACCTAT
251 TGAATGTTGT CACTGGACCT GAGGACATTC CCCATGCCGT CCTGATCCGG
301 GCCATCCTTC CTGATCAAGG CAAAGAACTT ATGATCCAAC GCCGCCAATG
351 GAGAGATAAA CCCCCACACC TTCTCACCAA TGGACCCGGA AAAGTGTGCC
401 AAGCTCTAGG AATCTCTTTG GAAAACAATA GGCAACGCCT AAATACCCCA
451 GCTCTCTATA TCAGCAAAGA AAAAATCTCT GGGACTCTAA CAGCAACTGC
501 CCGGATCGGC ATCGATTATG CTCAAGAGTA TCGTGATGTC CCATGGAGAT
551 TTCTCCTATC CCCAGAAGAT TCGGGAAAAG TTTTATCTTA A
```

The PSORT algorithm predicts cytoplasm (0.180).

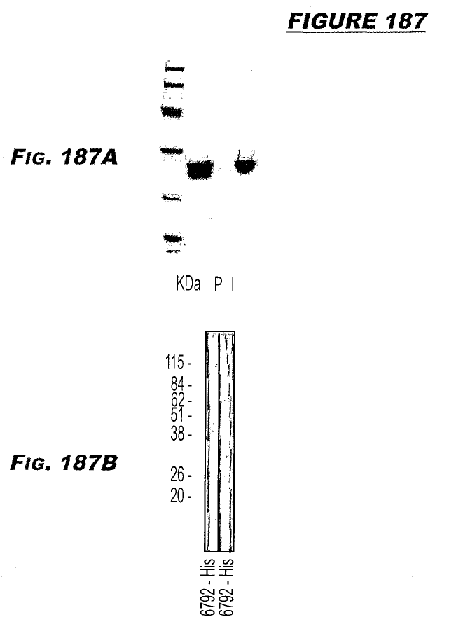

The protein was expressed in E. coli and purified as a his-tagged product (FIG. 187A; lanes 2-4). The recombinant protein was used to immunize mice, whose sera were used in a Western blot (FIG. 187B) and for FACS analysis.

These experiments show that cp6792 is a surface-exposed and immunoaccessible protein, and that it is a useful immunogen. These properties are not evident from the sequence alone.

### Example 188

The following C. pneumoniae protein (PID 4376868) was expressed <SEQ ID 375; cp6868>:

```
    1 MVETVLHNFQ RYLSKYLYRV FRFPCRKKTF LSSHRVLARP SFPVDYCPGK
  51 IYDLQEIYEE LNAQLFQGAL RLQIGWFGRK ATRKGKSVVL GLFHENEQLI
101 RIHRSLDRQE IPRFFMEYLV YHEMVHSVVP REYSLSGRSI FHGKKFKEYE
151 QRFPLYDPAV AWEKANAYLL RGYKKRVGGG YGPA*
```

The cp6868 nucleotide sequence <SEQ ID 376> is:

```
    1 ATGGTTGAAA CAGTACTTCA TAATTTCCAA CGTTATCTGA GCAAGTATCT
  51 CTATAGGGTA TTTCGCTTCC CATGTCGTAA AAAGACGTTC CTATCTTCGC
101 ACAGGGTTCT TGCTCGTCCT TCATTCCCAG TAGACTACTG TCCGGGAAAG
151 ATCTATGATT TGCAGGAGAT CTATGAGGAA TTGAATGCGC AGTTATTTCA
201 AGGTGCACTG CGTTTACAGA TTGGTTGGTT CGGAAGGAAA GCTACCAGAA
251 AAGGCAAGAG TGTTGTCTTG GGATTGTTTC ATGAAAATGA ACAGTTAATT
301 CGAATTCATC GTTCTTTAGA TCGGCAGGAA ATCCCAAGAT TTTTTATGGA
351 ATATCTTGTG TATCATGAAA TGGTTCATAG TGTAGTCCCT AGAGAGTATT
401 CTCTATCGGG GCGTTCGATT TTTCATGGTA AAAAGTTTAA AGAATACGAA
451 CAACGTTTCC CCTTGTATGA TCGTGCTGTT GCTTGGGAAA AGGCAAACGC
501 TTATTTATTG CGAGGGTATA AAAAAAGAGT AGGTGGAGGA TATGGCAGGG
551 CATAG
```

The PSORT algorithm predicts bacterial cytoplasm (0.325).

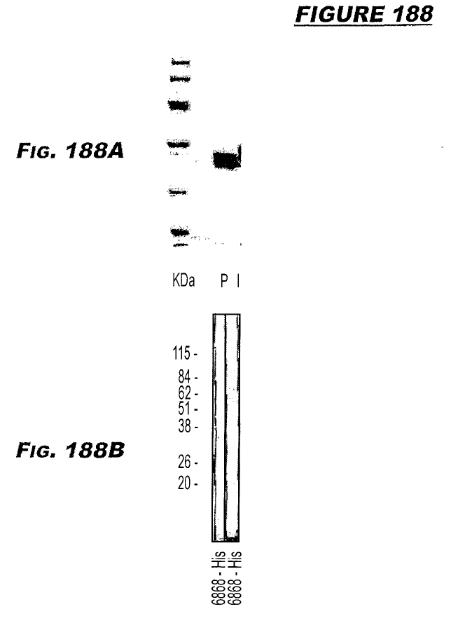

The protein was expressed in E. coli and purified as a his-tag product (FIG. 188A; lanes 2-3). The recombinant protein was used to immunize mice, whose sera were used in a Western blot (FIG. 188B) and for FACS analysis.

These experiments show that cp6868 is a surface-exposed and immunoaccessible protein, and that it is a useful immunogen. These properties are not evident from the sequence alone.

### Example 189

The following C. pneumoniae protein (PID 4376894) was expressed <SEQ ID 377; cp6894>:

```
    1 MYKRCVLDKI LKGIVAGSLI LLYWSSDLLE RDIKSIKGNV RDIQEDIREI
  51 SRVVKQQQTS QAIPAAPGVM LAPKLVRDEA FALLFGDPSY PNLLSLDPYK
101 QQTLPELLGT NFHPHGILRT AHVGKPENLS PFNGFDYVVG FYDLCIPSLA
151 SPHVGKYEEF SPDLAVKIEE HLVEDGSGDK EFHIYLRPNV FWRPIDPKAL
201 PKHVQLDEVF QRPHPVTAHD IKFFYDAVMN PYVATMRAVA LRSCYEDVVS
251 VSVENDLKLV VRWKAHTVIN EEGKEERKVL YSAFSNTLSL QELEREVYQY
301 FANGEKIIED ENIDTYRTNS IWAQNFTMHW ANNYIVSCGA YYFAGMDDEK
351 IVFSRNPDFY DPLAALIDKR FVYFKESTDS LFQDFKTGKI DISYLPPNQR
401 DNFYSFMKSS AYNKQVAKGG AVRETVSADR AYTYTGWNCF SLFFQSRQVR
451 CAMNMAIDRE RIIEQCLDGQ GYTISGEFAS SSPSYNKQIE GWHYSPEEAA
501 RLLEEEGWID TDGDGIREKV IDGVIVPFRF RLCYYVKSVT AHTIADYVAT
551 ACKEIGIECS LLGLDMADLS QAFDEKNFDA LLMGWCLGIP PEDPRALWHS
601 EGAMEKGSAN VVGFHNEEAD KIIDRLSYEY DLKERNRLYH RFHEIIHEEA
651 PYAFLFSRHC SLLYKDYVKN IFVPTHRTDL IPEAQDETVN VTMVWLEKKE
701 DPCLSTS*
```

The cp6894 nucleotide sequence <SEQ ID 378> is:

```
      1 ATGTATAAAA GATGTGTGCT AGATAAAATT TTAAAGGGGA TTGTCGCCGG
    51 TTCTTTAATT TTGTTATACT GGTCCTCAGA CCTACTTGAA AGAGACATTA
  101 AGTCGATAAA AGGTAACGTA AGAGATATTC AAGAAGACAT TCGTGAAATC
  151 TCACGCGTAG TGAAACAACA GCAGACATCA CAAGCTATCC CTGCGGCACC
  201 TGGGGTGATG CTCGCTCCTA AGCTCGTCAG AGACGAAGCT TTTGCTCTAC
  251 TCTTTGGAGA TCCTAGTTAT CCTAATTTAC TTTCCCTAGA CCCCTATAAA
  301 CAGCAGACTC TTCCTGAACT TCTAGGAACA AATTTCCACC CTCATGGTAT
  351 CCTACGCACT GCCCATGTCG GAAAACCCGA AAATCTGAGC CCTTTTAATG
  401 GCTTTGATTA TGTCGTGGGC TTTTACGATC TCTGTATTCC TAGTTTAGCT
  451 TCTCCCCACG TAGGGAAATA CGAAGAATTT TCTCCAGATC TCGCTGTGAA
  501 AATAGAAGAA CATCTTGTTG AAGATGGTTC TGGGGATAAA GAGTTTCACA
  551 TCTATCTGAG GCCGAATGTT TTTTGGCGTC CTATAGATCC TAAGGCCCTT
  601 CCAAAACACG TTCAGTTAGA CGAAGTATTT CAACGTCCTC ATCCTGTGAC
  651 AGCTCATGAT ATTAAGTTTT TCTACGACGC TGTTATGAAC CCTTATGTAG
  701 CAACCATGCG AGCAGTGGCT CTGCGCTCTT GTTATGAAGA TGTGGTTTCT
  751 GTCTCAGTAG AAAACGATTT AAAATTAGTA GTCAGATGGA AAGCACACAC
  801 GGTAATCAAT GAAGAAGGAA AGGAAGAGCG CAAAGTGCTC TACTCTGCAT
  851 TTTCTAATAC CTTAAGCTTG CAGCCCCTCC CTAGATTTGT ATATCAGTAT
  901 TTTGCTAACG GGGAAAAAAT CATTGAAGAT GAGAATATCG ATACCTACCG
  951 AACCAATTCC ATTTGGGCGC AAAACTTCAC TATGCATTGG GCAAACAACT
1001 ATATTGTAAG TTGTGGAGCC TACTACTTTG CAGGGATGGA TGATGAGAAA
1051 ATCGTGTTTT CTAGAAATCC TGACTTCTAT GATCCTCTTG CGGCTCTTAT
1101 TGACAAGCGT TTCGTCTATT TTAAGGAAAG CACAGACTCC CTATTCCAAG
1151 ATTTTAAGAC AGGGAAAATA GACATCTCTT ACCTTCCACC CAACCAAAGA
1201 GATAATTTCT ATAGTTTTAT GAAAAGCTCC GCTTATAACA AACAGGTAGC
1251 TAAGGGAGGA GCCGTCCGTG AAACAGTCTC AGCAGATCGA GCATATACGT
1301 ACATAGGATG GAATTGCTTT TCATTATTTT TCCAAAGCCG ACAGGTGCGC
1351 TGTGCTATGA ACATGGCAAT CGATAGAGAG AGGATTATCG AACAGTGCTT
1401 GGATGGCCAA GGCTATACGA TTAGTGGGCC TTTTGCTTCG AGTTCTCCTT
1451 CTTATAATAA ACAGATCGAA GGGTGGCATT ATTCTCCAGA AGAAGCAGCT
1501 CGTCTCCTGG AAGAAGAGGG ATGGATAGAT ACCGATGGCG ATGGAATCCG
1551 AGAAAAAGTT ATCGATGGTG TGATTGTCCC GTTCCGTTTC CGTTTATGCT
1601 ATTATGTAAA GAGTGTCACC GCTCATACCA TTGCAGATTA CGTAGCTACT
1651 GCTTGTAAGG AAATCGGAAT CGAGTGTAGC CTTCTAGGAC TAGATATGGC
1701 CGATCTTTCG CAAGCTTTTG ATGAAAAGAA TTTCGATGCT CTTTTAATGG
1751 GATGGTGTTT AGGAATTCCT CCTGAGGATC CTAGGGCTTT ATGGCATTCT
1801 GAAGGGGCTA TGGAAAAGGG TTCAGCGAAT GTTGTAGGTT TCCATAATGA
1851 AGAAGCTGAT AAAATCATAG ACAGACTCAG CTACGAATAC GATCTGAAAG
1901 AACGTAATCG CCTGTACCAC CGTTTCCATG AAATTATTCA TGAGGAAGCT
1951 CCTTATGCTT TCTTGTTCTC ACGACATTGT TCCTTACTTT ATAAGGATTA
2001 TGTAAAAAAT ATTTTCGTAC CTACACATAG AACAGATTTA ATTCCTGAAG
2051 CTCAGGATGA GACTGTCAAC GTAACTATGG TATGGCTTGA GAAGAAGGAG
2101 GATCCGTGCT TAAGTACATC CTAA
```

The PSORT algorithm predicts inner membrane (0.162).

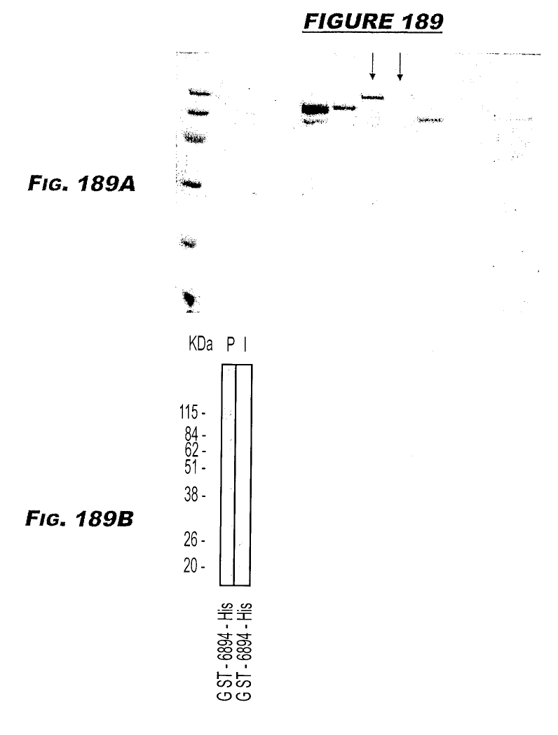

The protein was expressed in E. coli and purified as a his-tag product (FIG. 189A) and also in GST/his form. The recombinant proteins were used to immunize mice, whose sera were used in a Western blot (FIG. 189B) and for FACS analysis.

These experiments show that cp6894 is a surface-exposed and immunoaccessible protein, and that it is a useful immunogen. These properties are not evident from the sequence alone.

### Example 190

The following C. pneumoniae protein (PID 4377193) was identified in the 2D-PAGE experiment <SEQ ID 379; cp7193>:

```
    1 MKRVIYKTIF CGLTLLTSLS SCSLDPKGYN LETKNSRDLN QESVILKENR
  51 ETPSLVKRLS RRSRRLFARR DQTQKDTLQV QANFKTYAEK ISEQDERDLS
101 FVVSSAAEKS SISLALSQGE IKDALYRIRE VHPLALIEAL AENPALIEGM
151 KKMQGRDWIW NLFLTQLSEV FSQAWSQGVI SEEDIAAFAS TLGLDSGTVA
201 SIVQGERWPE LVDIVIT*
```

A predicted leader peptide is underlined.

The cp7193 nucleotide sequence <SEQ ID 380> is:

```
    1 ATGAAAAGAG TCATTTATAA AACCATATTT TGCGGGTTAA CTTTACTTAC
  51 AAGTTTGAGT AGTTGTTCCC TGGATCCTAA AGGATATAAC CTAGAGACAA
101 AAAACTCGAG GGACTTAAAT CAAGAGTCTG TTATACTGAA GGAAAACCGT
151 GAAACACCTT CTCTTGTTAA GAGACTCTCT CGTCGTTCTC GAAGACTCTT
201 CGCTCGACGT GATCAAACTC AGAAGGATAC GCTGCAAGTG CAAGCTAACT
251 TTAAGACCTA CGCAGAAAAG ATTTCAGAGC AGGACGAAAG AGACCTTTCT
301 TTCGTTGTCT CGTCTGCTGC AGAAAAGTCT TCAATTTCGT TAGCTTTGTC
351 TCAGGGTGAA ATTAAGGATG CTTTGTACCG TATCCGAGAA GTCCACCCTC
401 TAGCTTTAAT AGAAGCTCTT GCTGAAAACC CTGCCTTGAT AGAAGGGATG
451 AAAAAGATGC AAGGCCGTGA TTGGATTTGG AATCTTTTCT TAACACAATT
501 AAGTGAAGTA TTTTCTCAAG CTTGGTCTCA AGGGGTTATC TCTGAAGAAG
551 ATATCGCCGC ATTTGCCTCC ACCTTAGGTT TGGACTCCGG GACCGTTGCG
601 TCCATTGTCC AAGGGGAAAG GTGGCCCGAG CTTGTGGATA TAGTGATAAC
651 TTAA
```

The PSORT algorithm predicts periplasmic (0.925).

This shows that cp7193 is an immunoaccessible protein in the EB and that it is a useful immunogen. These properties are not evident from the protein's sequence alone.

It will be appreciated that the invention has been described by way of example only and that modifications may be made whilst remaining within the spirit and scope of the invention.

TABLE II
sequences of the primers used to amplify Cpn genes.

| | | |
|---|---|---|
| Orf ID | N-terminus final primer | C-terminus final primer |

| | | |
|---|---|---|
| CP0014P | GCGTC CCG GGTCATATG AAGTCTTCTTTCCCCA | GCGT CTC GAG ATGAAAGAGTTTTTGCG |
| CP0015P | GCGTCCCGGGTCATATG TCAGCTCTGTTTTCTGA | GCGT CTC GAG GAATTGGTATTTTGCTC |
| CP0016P | GCGTCCCGGGTCATATG GCCGATCTCACATTAG | GCGT CTC GAG GTCCAAGTTAAGGTAGCA |
| CP0017P | GCGT CCG GGTCATATG GGTATCAAGGGAACTG | GCGT CTC GAG AAATCCGAATCTTCC |
| CP0019P | GCGTCCCGGGTCAT ATGCAAGACTCTCAAGACTATAG | GCGT CTC GAG AAATCGGTATTTACCC |
| CP6260P | GCGTC CCG GGT GCTAGCACTACGATTTCTTTAACCC | GCGT CTC GAG AAAACGAAATTTGCTTC |
| CP6397P | GCGTC CCG GGTCATATGTTTAAACTGCTAAAAAATCTATT | GCGT CTC GAG ATGAAAGAAGAGTCCTCG |
| CP6456P | GCGTC CCG GGT CATATG TCATCTCCTGTAAATAACA | GCGT CTC GAG CTGACCATCTCCTGTT |
| CP6466P | GCGTC CCG GGT CAT ATG TGCAAGGAGTCCAGT | GCGT CTC GAG ATTTTCCTTAGCATAACG |
| CP6467P | GCGTC CCG GGT CAT ATG TGTTCCCCATCCCAA | GCGT CTC GAG TAGTTTTTCTATAAAACGAAAGTCT |
| CP6468P | GCGTC CCG GGT CAT ATG TGCTCCTCCTACTCTTC | GCGT CTC GAG GGGGAAATAGGTATATTTGA |
| CP6469P | GCGTC CCG GGT CAT ATG AGCTGCTCAAAGCAA | GCGT CTC GAG ACTTAAGATATCGATATTTTTGA |
| CP6552P | GCGTC CCG GGT CAT ATG TGCCATAAGGAAGATG | GCGT CTC GAG ACCATTGTCTTGAGTCAT |
| CP6567P | GCGTC CCG GGT CAT ATG ACCTCACCGATCCCC | GCGT CTC GAG AGAAGCCGGTAGAGGC |
| CP6576P | GCGTC CCG GGT CAT ATG ACTGAAAAAGTTAAAGAAGG | GCGT CTC GAG GAA CATGCCCCCTAA |
| CP6727P | GCGTC CCG GGT CATATGCTACATCCACTAATGGC | GCGT CTC GAG GAAAGAATAACGAGTTCC |
| CP6729P | GCGTC CCG GGT CAT ATGGCAGATGCTTCTTTATC | GCGT CTC GAG GAATGAGTATCTTAGCC |
| CP6731P | GCGTC CCG GGT CATATGGCTGTTGTTGAAATCAAT | GCGTC CAT GGC GGC CGC GAACTGGAACTTACCTCC |
| CP6736P | GCGTC CCG GGT GCT AGCGTAGAAGTTATCATGCCTT | GCGTC CAT GGC GGC CGC AAATCGTAATTTGCTTC |
| CP6737P | GCGT GGA TCC CAT ATG GAGACTAGACTCGGAGG | GCGT CTC GAG AAATGTGGATTTTAGTCC |
| CP6751P | GCGTC CCG GGT GCT AGC AATGAAGGTCTCCAACT | GCGT CTC GAG AAATCTCATTCTACTCGC |
| CP6752P | GCGTGA ATT CAT ATGTTCGGGATGACTCCT | GCGT CTC GAG GAATTTTAAGGTACTTCCTG |
| CP6753P | GCGTC CCG GGT GCT AGCACTCCCTACTCTCATAGAG | GCGT CTC GAG AAACTTAAAGGTCGTTC |
| CP6767P | GCGTC CCG GGT CAT ATG ATAAAACAAATAGGCCGT | GCGT CTC GAG TTCGTAAGCAACTTCAGA |
| CP6829P | GCGTC CCG GGT CAT ATG AAGCAGATGCGTCTTT | GCGTC CAT GGC GGC CGC GAAACTAAGGGAGAGGC |
| CP6830P | GCGTC CCG GGT CAT ATG GATCCCGCGTCTGTT | GCGTC CAT GGC GGC CGC GAATACAAACCGGATCC |
| CP6832P | GCGTC CCG GGT CAT ATG CATAAAGTAATAGTTTTCATTT | GCGT CTC GAG TAAACTAGAAAAAGTCGTC |
| CP6848P | GCGTC CCG GGT CAT ATG TCATCAAATCTACATCCC | GCGT CTC GAG AACGCGAGCTATTTTAC |
| CP6849P | GCGTC CCG GGT GCT AGC AGCGGGGGTATAGAG | GCGT CTC GAG ATACACGTGGGTATTTTC |
| CP6850P | GCGTC CCG GGT CAT ATG TGCCGCATTGTAGAT | GCGT CTC GAG CTGTTTGCATCTGCC |
| CP6854P | GCGTC CCG GGT GCT AGC TCAATAGCTATTGCAAG | GCGT CTC GAG TTATCGAAATGTCTTTG |
| CP6879P | GCGTC CCG GGT CAT ATG GCAACACCCGCTCAA | GCGTC CAT GGC GGC CGC TCCTTGAAATTGCTCTTGC |
| CP6894P | GCGTC CCG GGT CAT ATG TATAAAAGATGTGTGCTAGA | GCGT CTC GAG GGATGTACTTAAGCACG |
| CP6900P | GCGTC CCG GGT CAT ATG AAGATAAAATTTTCTTGGAAG | GCGT AAG CTT GGGAAGACGATACCG |
| CP6952P | GCGTC CCG GGT CAT ATG CTCTCGGATCAATATATAGG | GCGT CTC GAG TCGAATTTCTTTTTTAGC |
| CP7034P | GCGTC CCG GGT CAT ATG AAAAAACAGGTATATCAATG | GCGT AAG CTT AAACGCTGAAATTATACC |
| CP7090P | GCGTC CCG GGT CAT ATG TGTAGCCTTTCCCCT | GCGT CTC GAG GCGTGCATGAATCTTA |
| CP7091P | GCGTC CCG GGT CAT ATG GAAGAATTAGAAGTTGTTGT | GCGT CTC GAG TAGTGTTCTCTTTATCGGT |
| CP7170P | GCGTC CCG GGT CAT ATG CTAGGGGCTGGAAACC | GCGT AAG CTT AAACTGCAGACCTGACG |
| CP7228P | GCGTC CCG GGT CAT ATG ACTGCTGTTCTTATTCTTACA | GCGT CTC GAG ATCTGAAAGCGGAGG |
| CP7249P | GCGTC CCG GGT CAT ATG ATCCCATCCCCTACC | GCGT CTC GAG ATCAGGTTGCTGAGACTT |
| CP7250P | GCGTC CCG GGT CAT ATG AATCTTTCAAACAGGTCT | GCGT CTC GAG ATTTTTTCTAGAGAGACTCTC |
| CP0018P | GTGCGT CATATG GCAACCACTCCACTAA | ACTCGCTA GCGGCCGC TAATGAGGTCCCCAG |
| CP6270P | GTGCGT CATATG AATTTATTAGGAGCTGCT | ACTCGCTA GCGGCCGC AAATTTGATTTTGCTACC |
| CP6735P | GTGCGT CATATG GCAGCACAAGTTGTATAT | ACTCGCTA GCGGCCGC TGGCGTAGAAGTGATC |
| CP6998P | GTGCGT CATATG TTGCCTGTAGGGAAC | ACTCGCTA GCGGCCGC GAATCTGAACTGACCAGA |
| CP7033P | GTGCGT CATATG GTTAATCCTATTGGTCCA | ACTCGCTA GCGGCCGC TTGGAGATAACCAGAATATA |
| CP7287P | GTGCGT CATATG TTACACAGCTCAGAACTAGA | ACTCGCTA GCGGCCGC GAAAATAATACGGATACCA |
| CP0010P | GTGCGT CATATG GCAACTGCTGAAAATATA | GCGT CTCGAG GAATTGGAACTTACCC |
| CP0468P | GTGCGT GCTAGC ATTTTTTATGACAAACTCTAT | GCGT CTCGAG AAATGTGCAATGACTCT |
| CP6272P | GTGCGT CATATG TTGACTCATCAAGAGGCT | GCGT CTCGAG GAAGGGAGGTTTTTTAGGT |
| CP6273P | GTGCGT CATATG ACATATCTGGAAGCTC | ACTCGCTA GCGGCCGC CTCCACAATTTTTATG |
| CP6362P | GTGCGT CATATG CCCTTTGATATTACTTATTATACA | GCGT CTCGAG TCGTTTCCAAATCCA |
| CP6372P | GTGCGT CATATG AAACAACACTATTCTCTAAATA | GCGT CTCGAG TTTCTTGTGGTTTTTCT |
| CP6390P | GTGCGT CATATG CGAGAGGTGCCTAAG | ACTCGCTA GCGGCCGC TCTCCTAGACAGCCTT |
| CP6402P | GTGCGT CATATG AATGTTGCGGATCTCCTTT | GCGT CTCGAG GAAGGGGTTGGCCGT |
| CP6446P | GTGCGT CATATG TGTAATCAAAAGCCCTCTT | GCGT CTCGAG GGGCTGAGGAGGAAC |
| CP6520P | GTGCGT GCTAGC AAACACTACCTATCATTTTCT | GCGT CTCGAG CAGAAAGGCTTTTCTTT |
| CP6577P | GTGCGT CATATG AATTTAGGCTATGTTAATTTA | GCGT CTCGAG GTTTTGTTTTTTGAAAGA |
| CP6602P | GTGCGT CATATG GCAGCATCAGGAGGCA | GCGT CTCGAG TGACCAAGGATAGGGTTTAG |
| CP6607P | GTGCGT CATATG CCTCGTGGTGACACTTT | GCGT CTCGAG CGCTGCTTCTTGCTC |
| CP6615P | GTGCGT CATATG TGCTCTCAAAAAACGACAA | GCGT CTCGAG TGAAGAGGCGCCATC |
| CP6624P | GTGCGT CATATG GATGCGAAAATGGGA | GCGT CTCGAG TCTTTGACATTCAAGAGC |
| CP6672P | GTGCGT CATATG ATTCCTACCATGTTAATG | GCGT CTCGAG GTCATACAATTTCCTTATATA |
| CP6679P | GTGCGT CATATG TGCACTCACTTAGGCT | GCGT CTCGAG CGAGTAGTTAGCACAAAC |
| CP6717P | GTGCGT GCTAGC AAGACAATCGTAGCTTCA | ACTCGCTA GCGGCCGC GGCTGGCATATAGGT |
| CP6784P | GTGCGT GCTAGC AAATCAAGATGTTCTATTGATA | GCGT CTCGAG TCCAAAACAACCCTCT |
| CP6802P | GTGCGT CATATG TGCGTAAGTTATATTAATTCCTT | GCGT CTCGAG CAGTCGGGCTTGTTG |
| CP6847P | GTGCGT CATATG TCGGATCTTTTACGAG | GCGT CTCGAG TTTTCTACACTGTTGTAATAAA |
| CP6884P | GTGCGT CATATG AATCAGCTGCTTTCT | GCGT CTCGAG AGAGAAGGTAATTGTACC |
| CP6886P | GTGCGT CATATG TGTCTACTTATTATCTATCTCTAC | GCGT CTCGAG TTCAGAAAAATGGCT |
| CP6890P | GTGCGT CATATG TCCCCACGACGACAA | GCGT CTCGAG TCCTGCAGCATTTAGC |
| CP6960P | GTGCGT CATATG TGTGACGTACGGTCTA | ACTCGCTA GCGGCCGC TTCACCTTGATTTCCT |
| CP6968P | GTGCGT CATATG TGCGATGCAAAAC | ACTCGCTA GCGGCCGC GGAAGTATGCTTAGATATT |
| CP6969P | GTGCGT CATATG TGCTGTGGTTACTCTATT | ACTCGCTA GCGGCCGC AAAAAGGTCATAGTATACCT |
| CP7005P | GTGCGT CATATG AAAACTGTGATATTGAACA | GCGT CTCGAG CTGAGCTTCTATTTCTATTAT |
| CP7072P | GTGCGT CATATG CCCATTTATGGGAAA | GCGT CTCGAG GTTGAGCAAAGGTTTG |
| CP7101P | GTGCGT CATATG TATTCGTGTTACAGCAA | GCGT CTCGAG GAAAAATTCTTTAGGGAG |
| CP7102P | GTGCGT CATATG GCCGCTAAAGCAAAT | GCGT CTCGAG TGAAAATGAAAGGATGGT |
| CP7105P | GTGCGT GCTAGC AGTCTATATCAAAAATGGTG | GCGT CTCGAG ATCTTTCATTTGGTTATCT |
| CP7106P | GTGCGT CATATG AAAGATTTGGGGACTCT | GCGT CTCGAG GAATCCTAAGGCATACCTA |
| CP7107P | GTGCGT GCTAGC AGTATAGTCAGAAATTCTGCA | GCGT CTCGAG GAAGCTAAGATTATAGCTACTTT |
| CP7108P | GTGCGT GCTAGC GCGGCCCTTTCCA | ACTCGCTA GCGGCCGC TTTATGTATATGGAACAGATAGG |
| CP7109P | GTGCGT CATATG GGACATTTTATTGATATTG | ACTCGCTA GCGGCCGC ATCATCAAGGTAGATAAAG |
| CP7110P | GTGCGT CATATG GGTTATTGCTATGTAATTACA | GCGT CTCGAG TTCTGATTGGACTCCA |
| CP7127P | GTGCGT CATATG GTGGCTTTAACGATAGC | ACTCGCTA GCGGCCG GCAGCCATCGTATTC |
| CP7130P | GTGCGT CATATG TTCAATATGCGAGG | GCGT CTCGAG CTTCTTATTTGAACTTTG |
| CP7140P | GTGCGT CATATG ACAGCCGGAGCAGCT | GCGT CTCGAG AGCACCCTCAATTTCATTG |
| CP7182P | GTGCGT CATATG GGATATGTTTTCTATGTGATC | GCGT CTCGAG GCTACTAAATCGAATCGA |
| CP6262P | GTGCGT CATATG ATCCCTGGATTAAGTTCA | ACTCGCTA GCGGCCGC TTCACTGGGAGCTTGA |
| CP6269P | GTGCGT CATATG TACCAGGAGAATCTAAGAT | ACTCGCTA GCGGCCGC GATTTTCTTCTTCAGCTC |
| CP6296P | GTGCGT CATATG GAGGAGGTGTCTGAGTAT | ACTCGCTA GCGGCCGC ATGTTTCTTTTTACTCTTTCT |
| CP6419P | GTGCGT CATATG GCTCCAGTCCGTGTT | GCGT CTCGAG AAGTGTTCGTTGGAAGT |
| CP6601P | GTGCGT CATATG AATAAGCTACTCAATTTCGT | GCGT CTCGAG GAAAATCTGAATTCTTCCT |
| CP6639P | GTGCGT CATATG TTAAATTCAAGCAATTCA | GCGT CTCGAG AGGAACTAAAACCTCATCT |
| CP6664P | GTGCGT GCTAGC GTTTTATTTCATGCTCAA | ACTCGCTA GCGGCCGC CTTAGAAAGACTATTTTCTAAGTA |
| CP6696P | GTGCGT CATATG TGCGTGATAATGGG | GCGT CTCGAG ATTCATCTTCGTAAAGAAT |
| CP6757P | GTGCGT CATATG GCAGTTGGTGGCGT | ACTCGCTA GCGGCCGC CTGTCCCTCTGGAGC |
| CP6790P | GTGCGT GCTAGC AGTGAACACAAAAAATCA | ACTCGCTA GCGGCCGC CTTATCGTCGTTATCAATA |
| CP6814P | GTGCGT CATATG CATGACGCACTTCTAAG | GCGT CTCGAG TACAGCTGCGCGA |
| CP6834P | GTGCGT CATATG GTTATGGGAACCTATATCG | GCGT CTCGAG TACATTTGTATTGATTTCAG |
| CP6878P | GTGCGT CATATG AACGTCCCTGATTCC | GCGT CTCGAG GCTAGCGGCTCTTTC |
| CP6892P | GTGCGT CATATG CAGAAGCATCCTTCCT | ACTCGCTA GCGGCCGC TCCTCTTTAGGAAATGG |
| CP6909P | GTGCGT CATATG TCCTCTTTAGGAAATGG | GCGT CTCGAG CAGTGCCAAGTAGGGA |
| CP7015P | GTGCGT CATATG GCAGTACGATTAATTGTTG | GCGT CTCGAG TTTATTGTAGTCTATTTTATATTTC |
| CP7035P | GTGCGT GCTAGC AGCAGAAAAGACAATGA | GCGT CTCGAG ATTTTGAGTGTCTTGCA |
| CP7073P | GTGCGT CATATG ATTACCATAAATCACGTG | GCGT CTCGAG TATCCATCGACTTATAGC |
| CP7085P | GTGCGT GCTAGC TGTATTTTCCCTTACGTA | ACTCGCTA GCGGCCGC GGATTCTGCATACTCTG |
| CP7092P | GTGCGT CATATG TCTCCTCTTCCTAAAAAA | GCGT CTCGAG GGATTCATTACTGACCA |
| CP7093P | GTGCGT CATATG AAATACCGCTTCACG | GCGT CTCGAG ATTCTGTAGGGCTACGT |
| CP7094P | GTGCGT CATATG GTACACTTCTCTCATAACCC | GCGT CTCGAG TAAGTTTGTATTGCGGTAT |
| CP7132P | GTGCGT CATATG TTGTTATTAGGGACTTTAGGA | GCGT CTCGAG TTTCCCAACCGCA |
| CP7133P | GTGCGT CATATG GCTGCGAATGCTC | GCGT CTCGAG TAATTTAATACTCTTTGAAGG |
| CP7177P | GTGCGT CATATG CCTACTCAAGTTAAAACAGA | GCGT CTCGAG AAGTTTATATTTCAGCACTT |
| CP7184P | GTGCGT GCTAGC CATATAGGATTTTGCCA | GCGT CTCGAG GTACTTAGCAAAGCGAT |
| CP7206P | GTGCGT GCTAGC AAGAAGCTATATCACCCTA | GCGT CTCGAG CACACCGAGGAAAC |
| CP7222P | GTGCGT CATATG GTAGTTTCAGAAGAAAAAGTC | GCGT CTCGAG ACGTATGCGCAACTG |
| CP7223P | GTGCGT CATATG GAAGTATTAGACCGCTCT | GCGT CTCGAG CGAGAAAAAGCTTCC |
| CP7224P | GTGCGT CATATG ATGAAGAAAATTCGAAA | ACTCGCTA GCGGCCGC TAAGCATTCACAAATGA |
| CP7225P | GTGCGT CATATG CATATTTTGCTTGATCGT | GCGT CTCGAG TCTTTTAACTAAATCTTGTTCTT |
| CP7303P | GTGCGT CATATG CTTGTCTATTGTTTTGATCC | GCGT CTCGAG AAAATATACGGAACTCGC |
| CP7304P | GTGCGT GCTAGC GAAGTTTATAGTTTTTCCC | GCGT CTCGAG TTTTTGATTCCTTAAGAAG |
| CP7305P | GTGCGT CATATG GAAGTTTATAGTTTTCACCCT | GCGT CTCGAG ACTCCTTGAGAAGGGAA |
| CP7307P | GTGCGT CATATG CTTAATCATGCTAAAAAGC | ACTCGCTA GCGGCCGC CTCTTTTATTTTAGGAAGCT |
| CP7342P | GTGCGT CATATG AAAAAAAAATTTATTTTCTACT | ACTCGCTA GCGGCCGC CACACTCTGTTCTTCTG |
| CP7347P | GTGCGT CATATG TTTTCTAAGGATTTGACTAA | GCGT CTCGAG CGAAGCAGAAGTCGT |
| CP7353P | GTGCGT CATATG AATATGCCTGTTCCTTCT | GCGT CTCGAG GGGGCGTAGGTTGTA |
| CP7193P | GTGCGT CATATG TGTTCCCTGGATCCT | ACTCGCTA GCGGCCGC AGTTATCACTATATCCACAAG |
| CP7248P | GTGCGT GCTAGC CTTGAACATTCTAAACAAGAT | GCGT CTCGAG ACGTAGTTTAAGAGCAGACT |
| CP7261P | GTGCGT CATATG TGTCTATCTGCCTACATAG | GCGT CTCGAG TTTTGATGCTTCTTTCA |
| CP7280P | GTGCGT CATATG GACCAGAAAATTGAAAA | GCGT CTCGAG AGAGGTCTTCTGAGTGC |
| CP7302P | GTGCGT CATATG AATTTCCATTGTAGTGTAGT | GCGT CTCGAG GAACAGTTCGATTTGTG |
| CP7306P | GTGCGT CATATG CTTCCTTTATCAGGGCA | ACTCGCTA GCGGCCGC TTCTTCAGGTTTCAGG |
| CP7367P | GTGCGT GCTAGC CGTTATGCCGAGGTC | GCGT CTCGAG TTCGTGCATTTGGTG |
| CP7408P | GTGCGT CATATG TTGAAAATCCAGAAAAA | GCGT CTCGAG ATTCATTTTCGGAAGAG |
| CP7409P | GTGCGT CATATG AGACGTTATCTTTTCATGGT | GCGT CTCGAG CCCTTTGCTCTTTACATAG |
| CP6733P | GTGCGT ACTAGT TGTCACCTACAGTCACTAG | GCGT CTCGAG GAATCGGAGTTTGGTA |
| CP6728P | GTGCGT ACTAGT AAGTCCTCTGTCTCTTGG | GCGT CTCGAG GAAACAAAACTTAGAGCCC |

TABLE III
Proteins with best results in FACS analysis

Molecular Weight (kDa)

| cp number | Theoretical | Western Blot | Fusion type |
|---|---|---|---|
| 6260 | 97.5 | 94; 70 | GST |
| 6270 | 87.5 | — | GST |
| 6272 | 78.0 | 90 | GST |
| 6273 | 58.6 | 74; 64; 50 | GST |
| 6296 | 31.1 | — | GST |
| 6390 | 88.9 | 102 | GST |
| 6456 | 42.5 | 89; 67, 45 | GST |
| 6466 | 57.5 | 59; 56 | His |
| 6467 | 59.0 | 67 | GST |
| 6552 | 28.4 | 50; 27 | GST |
| 6576 | 86.0 | 79; 70; 62; 45 | GST |
| 6577 | 17.3 | 12 | GST |
| 6602 | 43.4 | 53; 42; 34 | GST |
| 6664 | 54.5 | 104; 45 | GST |
| 6696 | 47.9 | 95; 53 | GST |
| 6727 | 130.0-142.9 | 123; 61; 39 | His |
| 6729 | 94.8 | multiple bands | GST |
| 6731 | 95.5 | 97 | GST |
| 6733 | 97.1 | 104 | His |
| 6736 | 100.1 | 98; 93; 66; 60 | GST |
| 6737 | 101.2 | multiple bands | GST |
| 6751 | 100.2 | 95; 71 | GST |
| 6752 | 102.1 | 97; 48 | His |
| 6767 | 29.1 | 28 | GST |
| 6784 | 32.9 | 35 | GST |
| 6790 | 71.3 | multiple bands | His |
| 6802 | 29.7 | — | GST |
| 6814 | 29.6 | 28 | GST |
| 6830 | 177.4 | 174; 91; 13 | GST |
| 6849 | 57.3 | multiple bands | GST |
| 6850 | 7.4-9.4 | 61; 14; 8 | GST |
| 6854 | 42.2 | — | GST |
| 6878 | 40.4 | — | GST |
| 6900 | 28.0 | — | GST |
| 6960 | 25.6 | 75; 35 | GST |
| 6968 | 34.6 | 83; 53; 35 | GST |
| 6998 | 39.3 | multiple bands | GST |
| 7033 | 68.2 | multiple bands | GST |
| 7101 | 113 | 105 | GST |
| 7102 | 63.4 | — | GST |
| 7105 | 29.2 | 30 | GST |
| 7106 | 39.5 | 72; 46 | GST |
| 7107 | 71.4 | 67; 31 | His |
| 7108 | 35.9 | 35 | GST |
| 7111 | 46.1 | 51 | GST |
| 7132 | 17.9 | 57; 47; 17 | His |
| 7140 | 36.2-29.8 | 50; 38; 34 | GST |
| 7170 | 34.4 | 77; 33 | GST |
| 7224 | 39.4 | 40 | GST |
| 7287 | 167.3 | 180 | GST |
| 7306 | 50.1 | 50 | GST |

TABLE IV
FACS-positive proteins not found in C. trachomatis
cp7105
cp7106
cp7107
cp7108
cp6390
cp6784
cp6296

TABLE V
Proteins identified by MALDI-TOF following 2D electrophoresis
cp6270
cp6552
cp6576
cp6577
cp6602
cp6664
cp6727
cp6728
cp6729
cp6733
cp6736
cp6737
cp6752
cp6767
cp6784
cp6790
cp6830
cp6849
cp6900
cp6960
cp6998
cp7033
cp7108
cp7111
cp7170
cp7287
cp7306

1. An isolated protein comprising amino acids 21-488 of the amino acid sequence SEQ ID NO:111.

2. The isolated protein of claim 1 which comprises the amino acid sequence SEQ ID NO:111.

3. The isolated protein of claim 1 which is a recombinant protein.

4. The isolated protein of claim 1 which is a fusion protein.

5. An isolated nucleic acid molecule which encodes the protein of claim 1.

6. An immunogenic composition, comprising:
the protein of claim 1; and
a pharmaceutically acceptable carrier.

7. The immunogenic composition of claim 6, further comprising an adjuvant.

8. The immunogenic composition of claim 7 wherein the adjuvant is selected from the group consisting of an aluminum salt, an oil-in-water emulsion, a saponin adjuvant, Freund's adjuvant, a cytokine, and an immunostimulating agent.

9. A method for inhibiting replication of Chlamydia pneumoniae in a host cell comprising administering to the host cell an immunologically effective amount of the isolated protein of claim 1, thereby inhibiting replication of Chlamydia pneumoniae in the host cell.

10. A method of eliciting an immune response to Chlamydia pneumoniae in a subject, comprising administering to a subject in need thereof an immunologically effective amount of an immunogenic composition of claim 6.

11. The method of claim 10 wherein the subject is a human.

12. A method of preparing an immunogenic composition, comprising combining the protein of claim 1 with a pharmaceutically acceptable carrier.
